(19) United States
(12) Patent Application Publication
(10) Pub. No.: US 2025/0222146 A1
(43) Pub. Date: Jul. 10, 2025

(54) Radiopharmaceuticals Targeting Somatostatin Receptor 2 and Uses Thereof

(71) Applicant: RayzeBio, Inc., San Diego, CA (US)

(72) Inventors: Abhijit Bhat, Carlsbad, CA (US); Nicholas D. Smith, San Diego, CA (US); Nicole Bakas, Poway, CA (US); Alain Noncovich, San Diego, CA (US); Andrew R. Hudson, San Diego, CA (US); Joe Anh Tran, San Diego, CA (US); Patrick Napier, San Diego, CA (US); Yalda Bravo, San Diego, CA (US)

(73) Assignee: RayzeBio, Inc., San Diego, CA (US)

(21) Appl. No.: 18/989,485

(22) Filed: Dec. 20, 2024

Related U.S. Application Data
(60) Provisional application No. 63/614,474, filed on Dec. 22, 2023.

(51) Int. Cl. A61K 51/08; C07K 7/54

(52) U.S. Cl. CPC A61K 51/083; A61K 51/088; C07K 7/54; A61K 2121/00; A61K 2123/00

(57) ABSTRACT

Provided herein are peptides and conjugates (e.g., radiopharmaceutical conjugates) having avidity for a somatostatin receptor and uses thereof. The conjugate can comprise a 6-mer peptide, such as a monocyclic 6-mer peptide, a metal chelator, and optionally a linker connecting the peptide to the chelator. The radiopharmaceutical conjugate can further comprise a radionuclide bound to the metal chelator. Further provided herein are methods of preparing the conjugates, and methods of treating cancer by administering the described conjugates to a subject in need thereof.

### CROSS-REFERENCE TO RELATED APPLICATION

This application claims the benefit of priority of U.S. Provisional Patent Application No. 63/614,474, filed Dec. 22, 2023, which is incorporated by reference herein in its entirety for all purposes.

### REFERENCE TO SEQUENCE LISTING

The present application is being filed with a Sequence Listing in electronic format. The sequence listing filed, entitled “01277-0073-00PCT-RYZ_SL.xml,” was created Dec. 18, 2024, and is 754,191 bytes in size. The information in electronic format of the Sequence Listing is incorporated herein by reference in its entirety.

### BACKGROUND

In the United States, cancer is the leading cause of death for those under 65 years of age, and it accounted for about 21% of all deaths in 2018. Many tumors express certain biomarkers such as somatostatin receptors (SSTRs). For example, neuroendocrine tumors (NETs) arise from neuroendocrine cells and most commonly develop in the lung, digestive tract and pancreas. NETs are one of the cancers that need systemic therapies because either they are inoperable, or they are diagnosed at an advanced stage with distant spread of tumor cells. Traditional radiotherapies such as external beam radiation therapy have been used for decades as a standard-of-care treatment for diagnosed cancer patients. While some patients respond to external beam radiation therapy, many others do not. Further, metastasis and circulating tumor cells can spread and remain in the bloodstream or bodily fluids after standard-of-care treatment and lead to resistance to therapy. The presence of cancer cells in various parts of the body reduces the therapeutic efficacy of traditional radiotherapies. Accordingly, strategies for targeted radiotherapies are being developed for better cancer treatment and diagnosis.

### SUMMARY

In one aspect, the present disclosure relates to a peptide having avidity for a somatostatin receptor, wherein the peptide comprises a structure of Formula (I) or a salt thereof,

X1-X2-X3-X4-X5-X6 Formula (I)
wherein,
X1 is any amino acid;
X2 is any amino acid;
X3 is a non-natural, aromatic amino acid;
X4 is a non-aromatic amino acid having a side chain comprising an amine;
X5 is any amino acid; and
X6 is any amino acid.
   In some embodiments, the peptide is a monocyclic peptide. In some embodiments, the peptide has a structure of Formula (II),

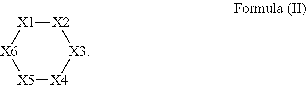

In one aspect, the present disclosure relates to a conjugate or a pharmaceutically acceptable salt thereof, wherein the conjugate comprises a monocyclic peptide having avidity for a somatostatin receptor, wherein the monocyclic peptide comprises a structure of Formula (II),

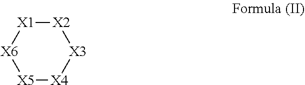

wherein,
X1 is any amino acid;
X2 is any amino acid;
X3 is a non-natural, aromatic amino acid;
X4 is a non-natural amino acid having a side chain comprising an amine;
X5 is any amino acid; and
X6 is any amino acid.
   In some embodiments, the conjugate further comprises a metal chelator covalently connected to the monocyclic peptide. In some embodiments, the metal chelator is connected to the monocyclic peptide through a linker. In some embodiments, the conjugate has a structure of Formula (III),

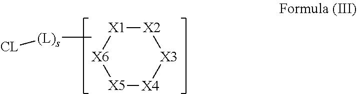

wherein,
L is a linker;
s is 0 or 1; and
CL is a metal chelator.

In some embodiments, CL-(L)ₛ- is attached to X1, X2, or X6. In some embodiments, s is 0 and the linker is a bond. In some embodiments, the conjugate further comprises a radionuclide bound to the metal chelator. In some embodiments, the metal chelator comprises DOTA, DOTA-GA, pBn-DOTA, pBn-SCN-DOTA, NH2-DOTA, NH2-DOTA-GA, p-NCS-Bn-DOTA-GA, p-NH2-Bn-oxo-DO3A, p-SCN-Bn-oxo-DO3A, NOTA, NODA-GA, NH2-NODA-GA, p-NCS-Bn-NODA-GA, p-NH2-Bn-NOTA, p-SCN-Bn-NOTA, NCS-MP-NODA, NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA.

In some embodiments, wherein the metal chelator has a structure of

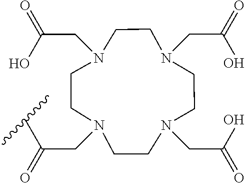 [SMILES: *C(=O)CN1CCN(CC(=O)O)CCN(CC(=O)O)CCN(CC(=O)O)CC1]

(DOTA). In some embodiments, the radionuclide is an alpha particle-emitting radionuclide. In some embodiments, the alpha particle-emitting radionuclide is Ac-225, At-211, Bi-213, Bi-209, Tb-149, Ra-223, Th-227, Fr-223, Gd-148, Th-229, Pb-212, or Po-213. In some embodiments, the alpha particle-emitting radionuclide is Ac-225, Bi-213, Bi-209, Tb-149, Ra-223, Th-227, Fr-223, Gd-148, Th-229, Pb-212, or Po-213. In some embodiments, the alpha particle-emitting radionuclide is Ac-225. In some embodiments, the radionuclide is a beta particle-emitting radionuclide. In some embodiments, the beta particle-emitting radionuclide is Cu-67, Lu-177, Y-90, Rh-105, Yb-175, Tm-167, Pm-153, Sm-153, or In-111. In some embodiments, the beta particle-emitting radionuclide is lutetium-177. In some embodiments, the radionuclide is a positron-emitting radionuclide. In some embodiments, the positron-emitting radionuclide is Ga-68, Cu-62, Cu-64, Zr-89, Tb-152.

In some embodiments, X1 is an N-methylated amino acid. In some embodiments, X1 is any amino acid comprising a polar side chain. In some embodiments, X1 is an L-amino acid. In some embodiments, X1 is Cys, Lys, Ala, Glu, Asp, Ser, Pro, or a derivative thereof. In some embodiments, X1 is Cys, Lys, Ala, Glu, Asp, Ser, or a derivative thereof. In some embodiments, X1 is not Pro or a derivative thereof. In some embodiments, X1 has a structure of:

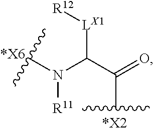

wherein,
R¹¹ is hydrogen or C₁-C₅alkyl optionally substituted with one to three substituents independently selected from R^{f};
each R^{f} is independently halogen, —CN, —NO₂, —OR^{a}, —SR^{a} or —NR^{c}R^{d};
R¹² is C₁-C₆alkyl, C₁-C₆hydroxyalkyl, C₁-C₆aminoalkyl, C₁-C₆heteroalkyl, C₃-C₆cycloalkyl, or 3- to 6-membered heterocycloalkyl, wherein each of the alkyl, heteroalkyl, cycloalkyl, and heterocycloalkyl is optionally substituted with one or more R^{12a},
L^{X1} is a bond, —O—, —S—, —NR¹³—, C₁-C₆alkylene, or C₁-C₆heteroalkylene, wherein the alkylene or heteroalkylene is optionally substituted with one or more R^{X1a}, or
R¹¹ and L^{X1}-R¹² are taken together with the intervening atoms to form a 5- to 6-membered heterocycloalkyl, which is optionally substituted with one or more R^{12a}.
each R^{12a} is independently halogen, C₁-C₆alkyl C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —SeR^{a}, —S(═O)R^{a}, —S(═O)₂R³, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, and heterocycloalkyl is optionally substituted with one or more R^{e}; or, one of R^{12a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R¹³ is hydrogen or C₁-C₃alkyl;
R^{X1a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X1a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O), NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X6 represents the point of attachment to X6; and
*X2 represents the point of attachment to X2.

In some embodiments, X2 is an aromatic amino acid. In some embodiments, X2 is Tyr, Phe, Trp, His, Gly, Ala, or a derivative thereof. In some embodiments, X2 has a structure of:

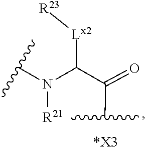

wherein:
R²¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d}.
L^{X2} is a bond, —O—, —S—, —NR²³—, C₁-C₆alkylene, or C₁-C₆heteroalkylene, wherein the alkylene or heteroalkylene is optionally substituted with one or more R^{X2a};
R²³ is hydrogen, C₁-C₆alkyl, C₁-C₆haloalkyl, C₁-C₆hydroxyalkyl, C₁-C₆aminoalkyl, or C₁-C₆heteroalkyl, wherein the alky and heteroalkyl is optionally substituted with one or more R^{e}; or
R²³ is

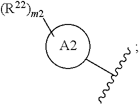

ring A2 is an aryl or heteroaryl;
each R²² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, alkenyl, alkynyl, aryl, heteroaryl, cycloalkyl, and heterocycloalkyl is optionally substituted with one or more R^{22a}, or
   two R²² are taken together to form ═O, ═S, or ═N(R^{a});
m2 is 0, 1, 2, 3, 4, or 5;
each R^{22a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR³)₂, —NR^{a}S(═O)₂NR^{c}R^{d}, —C(═O)R³, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, and heterocycloalkyl is optionally substituted with one or more R^{e}; or, one of R^{22a} is a conjugation group (CG);
R²³ is hydrogen or C₁-C₆alkyl;
R^{X2a} is halogen, —CN, —NO₂, —OR^{a}, —NR^{a}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X2a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC; —C₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; or, one of R^{e} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X1 represents the point of attachment to X1; and
*X3 represents the point of attachment to X3.

In some embodiments, X3 is a non-natural amino acid comprising an optionally substituted N-containing 5- to 10-membered heteroaryl. In some embodiments, X3 has a structure of:

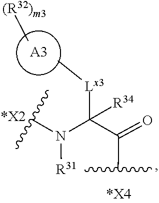

wherein:
R³¹ is hydrogen or C₁-C₅ alkyl optionally substituted with one to three substituents independently selected from R^{f};
each R^{f} is independently halogen, —CN, —NO₂, —OR^{a}, —SR^{a} or —NR^{c}R^{d};
L^{X3} is a bond, —O—, —S—, —NR³³—, C₁-C₃alkylene, or C₁-C₃heteroalkylene, wherein the alkylene or heteroalkylene is optionally substituted with one or more R^{X3a}.
ring A3 is an aryl or heteroaryl;
each R³² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, aryl, heteroaryl, cycloalkyl, and heterocycloalkyl is optionally substituted with one or more R^{32a}; or
two R³² are taken together to form ═O, ═S, or ═N(R^{a});
m3 is 0, 1, 2, 3, 4, or 5;
each R^{32a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR³)R², —N═S(═O)R^{c}R^{d}, —NR^{a}S(═O)₂R^{a}, amidinyl, —NR^{a}C(═NH)(NR³)₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or —N(R^{a}), wherein each of the alkyl, heteroalkyl, alkenyl, alkynyl, aryl, heteroaryl, cycloalkyl, or heterocycloalkyl is optionally substituted with one or more R^{e};
R³³ is hydrogen or C₁-C₃alkyl;
R³⁴ is hydrogen or C₁-C₃alkyl;
R^{X3a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X3a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X2 represents the point of attachment to X2; and
*X4 represents the point of attachment to X4.

In some embodiments, X3 has a structure of:

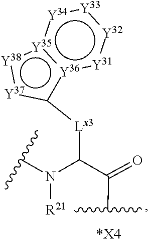

wherein:
Y³¹ is N, CH, or CR³²;
Y³² is N, CH, or CR³²;
Y³³ is N, CH, or CR³²;
Y³⁴ is N, CH, or CR³²;
Y³⁵ is N, or C;
Y³⁶ is N or C;
Y³⁷ is N, CH, or CR³²; and
Y³⁸ is O, S, N or NH;
provided that no more than two of Y³¹, Y³², Y³³, Y³⁴, Y³⁵, Y³⁶, and Y³⁷ are N.

In some embodiments, X4 is a non-natural amino acid having a side chain comprising an amine. In some embodiments, the amine comprises a primary amine, secondary amine, tertiary amine, or quaternary amine. In some embodiments, the amine comprises a primary amine, secondary amine, or tertiary amine. In some embodiments, the amine comprises a primary amine. In some embodiments, the amine comprises a secondary amine. In some embodiments, the amine comprises a tertiary amine. In some embodiments, the amine comprises a quaternary amine. In some embodiments, X4 is a non-natural amino acid having a side chain comprising an amine, and wherein the side chain comprises azetidinyl, pyrrolidinyl, piperidinyl, piperazinyl, or morpholinyl. In some embodiments, X4 is a non-natural amino acid having a side chain comprising an amine, and the non-natural amino acid is a Lys derivative. In some embodiments, X4 has a structure of:

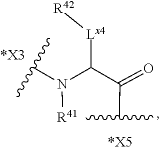

wherein,
R⁴¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
L^{X4} is a bond, —O—, —S—, —NR⁴³—, C₁-C₆alkylene, C₁-C₆heteroalkylene, C₃-C₆cycloalkyl, or 3- to 6-membered heterocycloalkyl, wherein the alkylene, heteroalkylene, cycloalkyl, or heterocycloalkyl is optionally substituted with one or more R^{X4a}.
R⁴² is —NR⁴⁴R⁴⁵ or a heterocycloalkyl comprising one or more ring nitrogen atoms, wherein the heterocycloalkyl is optionally substituted with one or more R^{42a},
each R^{42a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R³, —SF₅, —S(═O)₂NR^{c}R^{d}, —S(═O)(═NR³)R², —N═S(═O)R^{c}R⁴, —NR^{a}S(═O)₂R^{a}, amidinyl, —NR^{a}C(═NH)(NR³)₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR⁴), —P(═O)R^{c}R^{d}, ═O, ═S, or ═N(R³), wherein each of the alkyl, heteroalkyl, alkenyl, and alkynyl is optionally substituted with one or more R^{e};
R⁴³ is hydrogen or C₁-C₆alkyl;
R⁴⁴ and R⁴⁵ are each independently hydrogen, C₁-C₆alkyl, aryl, heteroaryl, —C₁-C₆alkylene-aryl, or —C₁-C₆alkylene-heteroaryl; or R⁴⁴ and R⁴⁵ are taken together to form a 3- to 6-membered heterocycloalkyl, wherein each of the alkyl, aryl, heteroaryl, and heterocycloalkyl are optionally substituted with one or more R^{42a};
each R^{X4a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R³, —S(═O)₂R^{a}, —SF₅, —S(═O)₂NR^{c}R^{d}, —S(═O)(═NR^{a})R³, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R³, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, ═O, ═S, or ═N(R^{a}), wherein each of the alkyl, heteroalkyl, alkenyl, and alkynyl is optionally substituted with one or more R^{e}; or or two R^{X4a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl), wherein each of the alkyl, alkylene, alkenyl, alkynyl, cycloalkyl, and heterocycloalkyl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC, —C₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, C₁-C₆alkylene(cycloalkyl), or C₁-C₆alkylene(heterocycloalkyl), wherein each of the alkyl, alkylene, alkenyl, alkynyl, cycloalkyl, and heterocycloalkyl, is independently optionally substituted with one or more R^{e}; or R^{c} and R^{d} are taken together with the atom to which they are attached to form a heterocycloalkyl optionally substituted with one or more R^{e};
*X3 represents the point of attachment to X3; and
*X5 represents the point of attachment to X5.

In some embodiments, X5 is an aliphatic amino acid or a polar amino acid. In some embodiments, X5 is Thr, Val, Ala, Ser, Pro, or a derivative thereof. In some embodiments, X5 has a structure of:

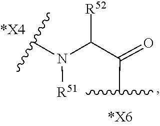

wherein,
R⁵¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
R⁵² is C₁-C₆alkyl, C₁-C₆haloalkyl, C₁-C₆hydroxyalkyl, C₁-C₆aminoalkyl, C₁-C₆heteroalkyl, C₂₋₆alkenyl, C₂₋₆alkynyl, C₃-C₆cycloalkyl, or 4- to 6-membered heterocycloalkyl, wherein each of the alkyl, heteroalkyl, aryl, heteroaryl, cycloalkyl, and heterocycloalkyl is optionally substituted with one or more R^{32a}, or
R⁵¹ and R⁵² are taken together with the intervening atoms to form a 5- to 6-membered heterocycloalkyl, which is optionally substituted with one or more R^{52a};
each R^{52a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —SeR^{a}, —S(═O)R^{a}, —S(═O)₂R^{a}, —SF₅, —S(═O)₂NR^{c}R^{d}, —S(═O)(═NR^{a}) R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR², —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R³), wherein each of the alkyl, heteroalkyl, alkenyl, alkynyl, aryl, heteroaryl, cycloalkyl, or heterocycloalkyl is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X4 represents the point of attachment to X4; and
*X6 represents the point of attachment to X6.

In some embodiments, X6 is an aromatic amino acid, a hydrophilic amino acid, or Gly, or a derivative thereof. In some embodiments, X6 is Phe, Ala, Gly, Ser, His, Tyr, Asn, Pro, or a derivative thereof. In some embodiments, X6 has a structure of:

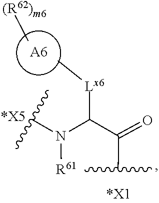

wherein:
R⁶¹ is hydrogen or C₁-C₆alkyl optionally substituted with one to three substituents independently selected from R^{f};
each R^{f} is independently halogen, —CN, —NO₂, —OR₃, —SR^{a} or —NR^{c}R^{d};
ring A6 is a cycloalkyl, heterocycloalkyl, aryl, or heteroaryl;
each R⁶² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R³, —S(═O)₂R^{c}R^{d}, —S(═O)(═NR³)R^{a}, —N═S(═O)R^{c}R^{d}, —NR^{a}S(═O)₂R^{a}, amidinyl, —NR^{a}C(═NH) NR^{c}R^{d}, —NR^{a}S(═O)₂R^{c}R^{d}, —C(═O)R^{a}, —C(═O)OR^{a}, —OC(═O)R³, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, aryl, heteroaryl, cycloalkyl, and heterocycloalkyl is optionally substituted with one or more R^{62a}, or
   two R⁶² are taken together to form ═O, ═S, or ═N(R^{a});
L^{X6} is a bond, —O—, —S—, —NR⁶³—, C₁-C₆alkylene, or C₁-C₆heteroalkylene, wherein the alkylene or heteroalkylene is optionally substituted with one or more R^{X6a}.
m6 is 0, 1, 2, 3, 4, or 5;
each R^{62a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{a}, —NR^{a}S(═O)₂R^{a}, amidinyl, —NR^{a}C(═NH)(NR³)₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R³, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{62a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R⁶³ is hydrogen or C₁-C₃ alkyl;
R^{X6a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X6a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C; —C(alkyl)₂, —NH₂, —NHC₁-C₆alkyl, —N(C₁-C₆alkyl)₂, —NHC(═NH)NH₂, —NHC(═O)OC₁-C₆alkyl, —C(═O)C; —C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X5 represents the point of attachment to X5; and
*X1 represents the point of attachment to X1.

In some embodiments, X6 has a structure of:

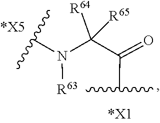

wherein:
R⁶³ is hydrogen or C₁-C₆alkyl optional substituted with one to three substituents independently selected from R^{f};
each R^{f} is independently halogen, —CN, —NO₂, —OR^{a}, —SR^{a} or —NR^{c}R^{d};
R⁶⁴ is hydrogen, C₁-C₆alkyl, C₁-C₆haloalkyl, C₁-C₆hydroxyalkyl, C₁-C₆aminoalkyl, C₁-C₆heteroalkyl, C₃-C₆cycloyalkyl, or 3- to 6-membered heterocycloalkyl, wherein each of the alkyl, heteroalkyl, cycloalkyl, and heterocycloalkyl is optionally substituted with one or more R^{64a},
R⁶⁵ is hydrogen, halogen, or C₁-C₆alkyl, wherein the alkyl is optionally substituted with one or more R^{64a}; or
R⁶⁴ and R⁶⁵ are taken together with the carbon to which they are attached to form a C₃-C₆cycloyalkyl or a 4- to 6-membered heterocycloalkyl, each of which is optionally substituted with one or more R^{64a}, or
R⁶³ and R⁶⁴ are taken together with the intervening atoms to form a 5- to 6-membered heterocycloalkyl, which is optionally substituted with one or more R^{64a};
each R^{64a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR³, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{64a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X5 represents the point of attachment to X5; and
*X1 represents the point of attachment to X1.

In some embodiments, X1 is NMe-Hcy, NMe-Lys, or NMe-hLys, NMe-Glu, or NMe-Amp; X2 is Tyr; X3 is D-Trp, (S-βMe) D-Trp, (S-βMe)-Trp, (R-βMe) D-Trp, or (R-βMe)-Trp; X4 is(S)-2-amino-3-(piperazin-1-yl)propanoic acid (PipzaA), (S)-2-amino-4-(azetidin-3-yl) butanoic acid (3-Azetidine-hAla), Lys(Me), (S)-2-amino-2-(piperidin-4-yl)acetic acid (Chg4N) or(S)-2-amino-3-(piperidin-4-yl)propanoic acid (Cha4N); X5 is Thr, Val, Ala, or Alt; and X6 is Phe.

In one aspect, the present disclosure relates to a pharmaceutical composition comprising a conjugate, or a pharmaceutically acceptable salt thereof, as described herein, and a pharmaceutically acceptable excipient or carrier.

In one aspect, the present disclosure relates to a method of treating a disease or disorder characterized by overexpression of SSTR, in a subject in need of treatment, the method comprising administering to the subject the conjugate or pharmaceutically acceptable salt thereof as described herein. In some embodiments, the disease or disorder is cancer. In some embodiments, the disease or disorder is a somatostatin receptor-positive (SSTR+) tumor. In some embodiments, provided herein is a method of killing an SSTR+ cell, comprising contacting the cell with a conjugate described herein.

In one aspect, the present disclosure relates to a kit, tester, or composition for determining the expression level of SSTR in a sample, wherein the kit, tester, or composition comprises the conjugate or pharmaceutically acceptable salt thereof as described herein.

In one aspect, the present disclosure relates to the use of the conjugate or pharmaceutically acceptable salt thereof as described herein in the manufacture of a medicament for diagnosing and/or treating a disease or disorder characterized by an overexpression or a decreased expression of SSTR.

In one aspect, the present disclosure relates to the use of the conjugate or pharmaceutically acceptable salt thereof as described herein for use in diagnosing and/or treating a disease or disorder characterized by an overexpression or a decreased expression of SSTR.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing (also “figure” and “FIG.” herein), of which:

FIG. 1A illustrates exemplary metal chelators of the present disclosure, wherein [symbol] represents the attachment point of a metal chelator to the remaining conjugate. FIG. 1B illustrates the same metal chelators as FIG. 1A, except that a part of the linker or the peptide covalently connected to the metal chelator is shown in the dashed circle [symbol] .

FIG. 2A illustrates exemplary metal chelators of the present disclosure, wherein [symbol] represents the attachment point of a metal chelator to the remaining conjugate. FIG. 2B illustrates the same metal chelators as FIG. 2A, except that a part of the linker or the peptide covalently connected to the metal chelator is shown in the dashed circle [symbol] .

FIG. 3A illustrates exemplary metal chelators of the present disclosure, wherein [symbol] represents the attachment point of a metal chelator to the remaining conjugate. FIG. 3B illustrates the same metal chelators as FIG. 3A, except that a part of the linker or the peptide covalently connected to the metal chelator is shown in the dashed circle [symbol] .

FIG. 4A illustrates exemplary metal chelators of the present disclosure, wherein [symbol] represents the attachment point of a metal chelator to the remaining conjugate. FIG. 4B illustrates the same metal chelators as FIG. 4A, except that a part of the linker or the peptide covalently connected to the metal chelator is shown in the dashed circle [symbol] .

FIG. 5A illustrates exemplary metal chelators of the present disclosure, wherein [symbol] represents the attachment point of a metal chelator to the remaining conjugate. FIG. 5B illustrates the same metal chelators as FIG. 5A, except that a part of the linker or the peptide covalently connected to the metal chelator is shown in the dashed circle [symbol] , and R represents hydrogen, alkyl (such as methyl) or other suitable group on the nitrogen.

FIG. 6 illustrates the structures of representative metal chelators.

FIG. 7 illustrates the structures of representative metal chelators.

FIG. 8 illustrates the structures of representative metal chelators.

FIG. 9 illustrates the structures of representative metal chelators.

FIG. 10 illustrates the structures of representative metal chelators.

FIG. 11 illustrates the structures of representative metal chelators.

FIG. 12 illustrates the structures of representative metal chelators.

FIG. 13 illustrates the structures of representative metal chelators.

FIG. 14 illustrates the structures of representative metal chelators.

FIG. 15 illustrates the structures of representative metal chelators.

FIG. 16 illustrates the structures of representative metal chelators.

FIG. 17 illustrates the structures of representative metal chelators.

FIG. 18 illustrates the structures of representative metal chelators.

FIG. 19 illustrates the structures of representative metal chelators.

FIG. 20 illustrates the structures of representative metal chelators.

FIG. 21 illustrates the structures of representative metal chelators.

FIG. 22 illustrates the structures of representative metal chelators.

FIG. 23 illustrates the structures of representative metal chelators.

FIG. 24 illustrates the structures of representative metal chelators.

FIG. 25 illustrates the structures of representative metal chelators.

### DETAILED DESCRIPTION

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

### I. Definitions

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents, reference to “a stabilizer” includes a plurality of such stabilizers, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Amine” refers to a moiety formally derived from ammonia (NH₃) by replacing one, two, or three hydrogen atoms. In some embodiments, the amine is a primary amine, a secondary amine, a tertiary amine, or a quaternary amine. In some embodiment, the amine is present in a heterocycloalkyl. In some embodiments, the amine is present in an aminoalkyl. In some embodiments, the amine is present in an alkylamino. In some embodiments, the amine is present in an amino group. In some embodiments, “Amine” refers to a moiety having the formula N(Rₐ)₂ where Rₐ is hydrogen or an alkyl radical as defined herein, or two Rₐ, taken together with the nitrogen atom, can form a substituted or unsubstituted C₂-C₇ heterocyloalkyl ring. In some embodiments, the side chain comprising an amine is a side chain comprising a primary amine, a secondary amine, a tertiary amine, or a quaternary amine. In some embodiments, the side chain comprising an amine is a side chain comprising a heterocycloalkyl. In some embodiments, the side chain comprising an amine is a side chain comprising an aminoalkyl. In some embodiments, the side chain comprising an amine is a side chain comprising an alkylamino.

“Amino” refers to the —NH₂ radical.

“Cyano” refers to the CN radical.

“Nitro” refers to the NO₂ radical.

“Oxo” refers to the ═O radical.

“Imino” refers to the ═N—H radical.

“Oximo” refers to the ═N—OH radical.

“Hydrazino” refers to the ═N—NH₂ radical.

“Hydroxy” or “hydroxyl” refers to the —OH radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, OH, —OMe, NH₂, —NO₂, or —C≡CH. In some embodiments, the alkyl is optionally substituted with oxo, halogen, —CN, —CF₃, OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, OH, —OMe, NH₂, or —NO₂. In some embodiments, an alkylene is optionally substituted with oxo, halogen, —CN, —CF₃, OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (CH═CH₂), 1-propenyl (CH₂CH═CH₂), isopropenyl [C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, OH, —OMe, NH₂, or —NO₂. In some embodiments, an alkenyl is optionally substituted with oxo, halogen, —CN, —CF₃, OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH—. In some embodiments, the alkenylene is —CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, OH, —OMe, NH₂, or —NO₂. In some embodiments, an alkynyl is optionally substituted with oxo, halogen, —CN, —CF₃, OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula N(Rₐ)₂ where Rₐ is an alkyl radical as defined herein, or two Rₐ, taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, OH, —OMe, NH₂, or —NO₂. In some embodiments, an alkylamino is optionally substituted with oxo, halogen, —CN, —CF₃, OH, or —OMe. In some embodiments, the alkylamino is optionally substituted with halogen.

“Alkoxy” refers to a radical of the formula ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, OH, —OMe, NH₂, or —NO₂. In some embodiments, an alkoxy is optionally substituted with oxo, halogen, —CN, —CF₃, OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. aminoalkyl include, for example, aminomethyl, aminoethyl, aminopropyl, aminobutyl, or aminopentyl. In some embodiments, the aminoalkyl is aminomethyl. An aminoalkyl group may be a primary aminoalkyl group, e.g., a radical of the formula-alkyl-NH₂, a secondary aminoalkyl group, e.g., a radical of the formula -alkyl-NHRₐ, a tertiary aminoalkyl group, e.g., a radical of formula-alkyl-N(Rₐ)₂, or a quaternary aminoalkyl group, e.g., a radical of formula-alkyl-N⁺(Rₐ)₃, wherein Rₐ is an alkyl radical as defined herein, or two Rₐ, taken together with the nitrogen atom, can form a substituted or unsubstituted C₂-C₇ heterocyloalkyl ring. Unless stated otherwise specifically in the specification, an aminoalkyl group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an aminoalkyl is optionally substituted with oxo, halogen, —CN, —CF₃, OH, —OMe, NH₂, or —NO₂. In some embodiments, an aminoalkyl is optionally substituted with oxo, halogen, —CN, —CF₃, OH, or —OMe. In some embodiments, the aminoalkyl is optionally substituted with halogen

“Hydroxyalkyl” refers to an alkyl radical, as defined above, that is substituted by one or more hydroxyl groups. In some embodiments, the alkyl is substituted with one hydroxyl. In some embodiments, the alkyl is substituted with one, two, or three hydroxyls. Hydroxyalkyls include, for example, hydroxymethyl, hydroxyethyl, hydroxypropyl, hydroxybutyl, or hydroxypentyl. In some embodiments, the hydroxyalkyl is hydroxymethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, OH, —OMe, NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, OH, —OMe, NH₂, or —NO₂.

The term “cycloalkyl” refers to a monocyclic or polycyclic non-aromatic radical, wherein each of the atoms forming the ring (i.e., skeletal atoms) is a carbon atom. In some embodiments, cycloalkyls are saturated or partially unsaturated. In some embodiments, a cycloalkyl is fully saturated. In some embodiments, a cycloalkyl is partially saturated (e.g., comprising more or more carbon-carbon double bond). In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). A cycloalkyl can comprise a fused, spiro or bridged ring system. In some embodiments, the cycloalkyl comprises a fused ring system. In some embodiments, the cycloalkyl comprises a spiro ring system. In some embodiments, the cycloalkyl comprises a bridged ring system. In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2trifluoroethyl, 1 fluoromethyl2fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of heteroalkyl are, for example, —CH₂—O—CH₃, —CH₂—N(alkyl)-CH₃, —CH₂—N(aryl)-CH₃—OCH₂CH₂OH, —OCH₂CH₂OCH₂CH₂OH, or —OCH₂CH₂OCH₂CH₂OCH₂CH₂OH. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, OH, —OMe, NH₂, or —NO₂. In some embodiments, a heteroalkyl is optionally substituted with oxo, halogen, methyl, ethyl, —CN, —CF₃, OH, or —OMe. In some embodiments, the heteroalkyl is optionally substituted with halogen.

As used herein, a “heteroalkylene” refers to divalent heteroalkyl group. Examples of such heteroalkylene are, for example, —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. In some embodiments, a heterocycloalkyl is fully saturated. In some embodiments, a heterocycloalkyl is partially saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2oxopiperazinyl, 2oxopiperidinyl, 2oxopyrrolidinyl, oxazolidinyl, piperidinyl, piperazinyl, 4piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1oxothiomorpholinyl, 1,1dioxothiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, OH, —OMe, NH₂, or —NO₂. In some embodiments, a heterocycloalkyl is optionally substituted with oxo, halogen, methyl, ethyl, —CN, —CF₃, OH, or —OMe. In some embodiments, the heterocycloalkyl is optionally substituted with halogen.

“Heteroaryl” refers to a ring system radical comprising carbon atom(s) and one or more ring heteroatoms that selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₀-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, OH, —OMe, NH₂, or —NO₂. In some embodiments, a heteroaryl is optionally substituted with halogen, methyl, ethyl, —CN, —CF₃, OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer.

In certain embodiments, “treating” includes the concepts of “alleviating”, which refers to lessening the frequency of occurrence or recurrence, or the severity, of any symptoms or other ill effects related to a disorder and/or the associated side effects. The term “treating” also encompasses the concept of “managing” which refers to reducing the severity of a particular disease or disorder in a patient or delaying its recurrence, e.g., lengthening the period of remission in a patient who had suffered from the disease.

The term “therapeutically effective amount” as used herein to refer to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “—O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

The term “optionally substituted” or “substituted” means that the referenced group is optionally substituted with one or more additional group(s). For example, “optionally substituted” or “substituted” can mean that the referenced group is optionally substituted with one or more substituents individually and independently selected from halogen, —CN, —NH₂, —NH (alkyl), —N(alkyl)₂, —OH, oxo, —CO₂H, —CO₂alkyl, —C(═O)NH₂, —C(═O)NH (alkyl), —C(═O)N (alkyl)₂, —S(═O)₂NH₂, —S(═O)₂NH (alkyl), —S(═O)₂N (alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, oxo, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, an “optionally substituted” group is independently substituted with 1-6 substituents selected from halogen, —CN, oxo, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₆alkyl, C₁-C₆alkoxy, C₁-C₆haloalkyl, C₁-C₆haloalkoxy, C₁-C₆hydroxyalkyl, C₁-C₆aminoalkyl, C₁-C₆heteroalkyl, aryl, heteroaryl, heterocycloalkyl and cycloalkyl. In some embodiments, an “optionally substituted” group is independently substituted with 1-6 substituents selected from halogen, —CN, oxo, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 5- to 6-membered heterocycloalkyl and C₃-C₆cycloalkyl. In some embodiments, an “optionally substituted” group is independently substituted with 1-6 substituents selected from halogen, oxo, —OH, —NH₂, —NHC₁-C₃alkyl, —N(C₁-C₃alkyl)₂, —C(═O)OH, —C(═O)NH₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, and C₃-C₆cycloalkyl. In some embodiments, optional substituents are independently selected from D, halogen, —CN, —NH₂, —OH, —NH(CH₃), —N(CH₃)₂, —NH (cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitutions, i.e. replacement of one hydrogen up to replacement of all hydrogens by substituents. In some embodiments, an “optionally substituted” group is unsubstituted. In some embodiments, an “optionally substituted” group is independently substituted with 1-6 substituents. In some embodiments, an “optionally substituted” group is independently substituted with 1-3 substituents. In some embodiments, an “optionally substituted” group is independently substituted with 1-2 substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

In the present disclosure, the term “amino acid” is used in its broadest meaning and it embraces not only natural amino acids but also derivatives thereof and unnatural amino acids. For example, the term “amino acid” encompasses unnatural or non-natural amino acids, and peptoids.

As used herein, the term “unnatural amino acid” or “non-natural amino acid” refers to an amino acid other than the 20 canonical amino acids. The 20 canonical amino acids refer to alanine (ala or A), arginine (arg or R), asparagine (asn or N), aspartic acid (asp or D), cysteine (cys or C), glutamine (gln or Q), glutamic acid (glu or E), glycine (gly or G), histidine (his or H), isoleucine (ile or I), leucine (leu or L), lysine (lys or K), methionine (met or M), phenylalanine (phe or F), proline (pro or P), serine (ser or S), threonine (thr or T), tryptophan (trp or W), tyrosine (tyr or Y), and valine (val or V). As used herein, canonical amino acids are L-amino acids.

In some embodiments, an amino acid described herein can be replaced with a derivative thereof. Examples of an amino acid derivatives include derivatives having an amine, amide, ester, or carboxyl group as the C-terminus and/or N-terminus thereof. An amino acid derivative further encompasses amino acid isomers, including D-amino acids. An amino acid derivative further encompasses alkylated amino acids, for example N-alkylation (e.g., N-methylation), beta-carbon alkylation or alpha-carbon alkylation. In some embodiments, a derivative of an amino acid is the amino acid with alpha-carbon alkylation. In some embodiments, a derivative of an amino acid is the amino acid with beta-carbon alkylation. In some embodiments, a derivative of an amino acid is the amino acid with N-alkylation. An amino acid derivative further encompasses the amino acids that have the same functional groups but with different lengths of the side chain (e.g., LysAc vs. OrnAc and cysteine vs. homocysteine). An amino acid derivative further encompasses amino acids with heteroatoms in the side chain (e.g., O-(aminomethyl)-homoserine is a derivative of lysine and serine). An amino acid derivative further encompasses amino acids containing conjugation groups, including azides and alkynes. For example, propargylglycine is a derivative of alanine, and azidolysine is a derivative of lysine. An amino acid derivative further encompasses amino acids with a different aromatic moiety compared to the canonical amino acid (e.g., the indole in tryptophan vs the 7-azaindole in 7-AzaTrp; the phenyl in phenylalanine vs the pyridine in 4Py). An amino acid derivative further encompasses amino acids with optional substituents, i.e., optionally substituted amino acid.

In some embodiments, an amino acid derivative refers to an optionally substituted amino acid. In some embodiments, an optionally substituted amino acid is optionally substituted with one or more substituents described herein. For example, in some embodiments, an optionally substituted amino acid is optionally substituted with one or more substituents independently selected from halogen, hydroxyl, cyano, amino, amide, nitro, ureido, C₁-C₆ alkyl, C₁-C₆ alkoxy, C₆-C₁₀ aryl, C₃-C₆ cycloalkyl, 6- to 10-membered heterocycloalkyl, and 6- to 10-membered heteroaryl. In some embodiments, the optionally substituted amino acid is optionally substituted with one or more substituents independently selected from halogen, —CN, —NH₂, —NH (alkyl), —N(alkyl)₂, oxo, —OH, —CO₂H, —CO₂alkyl, —C(═O)NH₂, —C(═O)NH (alkyl), —C(═O)N (alkyl)₂, —S(═O)₂NH₂, —S(═O)₂NH (alkyl), —S(═O)₂N (alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), SF₅, R^{b} OR^{a}, R^{b}OC(O)R^{a}, R^{b}OC(O)OR^{a}, R^{b}OC(O)N(R^{a})₂, R^{b}N(R^{a})₂, R^{b}C(O)R^{a}, R^{b}C(O)OR^{a}, R^{b}C(O)N(R^{a})₂, R^{b}OR^{c}C(O)N(R^{a})₂, R^{b}N(R^{a})C(O)OR^{a}, R^{b}N(R^{a})C(O)R^{a}, R^{b}N(R^{a})S(O)ₜR^{a} (where t is 1 or 2), R^{b}S(O)ₜR^{a} (where t is 1 or 2), R^{b}S(O)ₜOR^{a} (where t is 1 or 2), and R^{b}S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), R^{b}OR^{a}, R^{b}OC(O)R^{a}, R^{b}OC(O)OR^{a}, R^{b}OC(O)N(R^{a})₂, R^{b}N(R^{a})₂, R^{b}C(O)R^{a}, R^{b}C(O)OR^{a}, R^{b}C(O)N(R^{a})₂, R^{b}OR^{c}C(O)N(R^{a})₂, R^{b}N(R^{a})C(O)OR^{a}, R^{b}N(R^{a})C(O)R^{a}, R^{b}N(R^{a})S(O)ₜR^{a} (where t is 1 or 2), R^{b}S(O)ₜR^{a} (where tis 1 or 2), R^{b}S(O)ₜOR^{a} (where t is 1 or 2) and R^{b}S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), R^{b}OR^{a}, R^{b}OC(O)R^{a}, R^{b}OC(O)OR^{a}, R^{b}OC(O)N(R^{a})₂, R^{b}N(R^{a})₂, R^{b}C(O)R^{a}, R^{b}C(O)OR^{a}, R^{b}C(O)N(R^{a})₂, R^{b}OR^{c}C(O)N(R^{a})₂, R^{b}N(R^{a})C(O)OR^{a}, R^{b}N(R^{a})C(O)R^{a}, R^{b}N(R^{a})S(O)ₜR^{a} (where t is 1 or 2), R^{b}S(O)ₜR^{a} (where t is 1 or 2), R^{b}S(O)ₜOR^{a} (where tis 1 or 2) and R^{b}S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. Additional examples of amino acid/peptide derivatives include those obtained by modification such as phosphorylation, alkylation (e.g., methylation), acetylation, adenylylation, ADP-ribosylation, or glycosylation. These derivatives can be prepared by those skilled in the art in a known manner or a method based thereon.

In some embodiments, an amino acid comprising a cycloalkyl group can be a derivative of another amino acid having a cycloalkyl group. In some embodiments, an amino acid comprising a heterocycloalkyl group can be a derivative of another amino acid having a heterocycloalkyl group.

In some embodiments, a derivative of an amino acid is selected from amino acids that have similar polarity and/or charge with the amino acid. For example, in some embodiments, a polar, uncharged amino acid can be a derivative of another polar, uncharged amino acid (e.g., Hgn, Q, S, T, Qglucamine).

In some embodiments, a derivative of an amino acid has the same number of hydrogen donor as the amino acid. In some embodiments, a derivative of an amino acid has the same number of hydrogen acceptor as the amino acid.

In some embodiments, the amino acid derivative has a molecular weight that does not vary for more than 14, 28, 30, 45, or 60 g/mol compared to the amino acid. In some embodiments, the derivative has a molecular weight that does not vary for more than 14 g/mol compared to the amino acid. In some embodiments, the derivative has a molecular weight that does not vary for more than 50 g/mol compared to the amino acid. In some embodiments, the derivative has a molecular weight that does not vary for more than 28 g/mol compared to the amino acid.

An amino acid derivative further encompasses amino acids wherein a functional group is substituted with another functional group having similar properties, e.g., a cysteine can be substituted with a homocysteine. In some embodiments, an aryl functional group can be substituted with an aryl or heteroaryl group. In some embodiments, a heteroaryl functional group can be substituted with an aryl or heteroaryl group. In some embodiments, an amino functional group can be substituted with an NH (alkyl) group.

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain. For example, polypeptide chains can be linked by one or more disulfide bonds or associated by other means.

The term “peptide” as used herein refers to a compound that includes two or more amino acids. A peptide described herein can comprise one or more unnatural amino acids. The term “peptide” also encompasses peptide mimetics.

The term “peptide mimetic” or “mimetic” refers to biologically active compounds that mimic the biological activity of a peptide or a protein but are no longer entirely peptidic in chemical nature, e.g., they can contain non-peptide bonds (that are, bonds other than amide bonds between amino acids). As used herein, the term peptide mimetic is used in a broader sense to include molecules that are no longer completely peptidic in nature, such as pseudo-peptides, semi-peptides and peptoids. Whether completely or partially non-peptide, peptide mimetics described herein can provide a spatial arrangement of reactive chemical moieties that closely resemble the three-dimensional arrangement of active groups in the subject amino acid sequence or subject molecule on which the peptide mimetic is based. As a result of this similar active-site geometry, the peptide mimetic can have effects on biological systems that are similar to the biological activity of the subject entity.

In some embodiments, the peptide mimetics are substantially similar in both three-dimensional shape and biological activity to the subject amino acid sequence or subject molecule on which the peptide mimetic is based. Examples of methods of structurally modifying a peptide to create a peptide mimetic include the inversion of backbone chiral centers leading to D-amino acid residue structures that may, particularly at the N-terminus, lead to enhanced stability for proteolytical degradation without adversely affecting activity. An example is described in the paper “Tritiated D-ala1-Peptide T Binding”, Smith C. S. et al., Drug Development Res., 15, pp. 371-379 (1988). A second method is altering cyclic structure for stability, such as N to C interchain imides and lactames (Ede et al. in Smith and Rivier (Eds.) “Peptides: Chemistry and Biology”, Escom, Leiden (1991), pp. 268-270). An example of this is provided in conformationally restricted thymopentin-like compounds, such as those disclosed in U.S. Pat. No. 4,457,489. A third method is to substitute peptide bonds in the subject entity by pseudopeptide bonds that confer resistance to proteolysis.

The term “organic atoms” refers to atoms which would be found in organic compounds, such as carbon, hydrogen, nitrogen, oxygen, sulfur, phosphorus, fluorine, chlorine, bromine, or iodine. In some embodiments, an organic atoms refers to carbon, nitrogen, oxygen, sulfur, or phosphorus.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, C₁-Cₓ (or C₁-x) includes C₁-C₂, C₁-C₃ . . . C₁-Cx. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

The term “cyclized” or “cyclization” as used herein means that two amino acids apart from each other by at least one amino acid bind directly or bind indirectly to each other in one peptide to form a cyclic structure in the molecule. In some cases, the two amino acids bind via a linker or the like.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity-(the number of identical residues)/(alignment length in amino acid residues)*100. For this calculation, alignment length includes internal gaps but does not include terminal gaps.

It is appreciated that certain features of the disclosure, which are, for clarity, described in the context of separate embodiments, can also be provided in combination in a single embodiment.

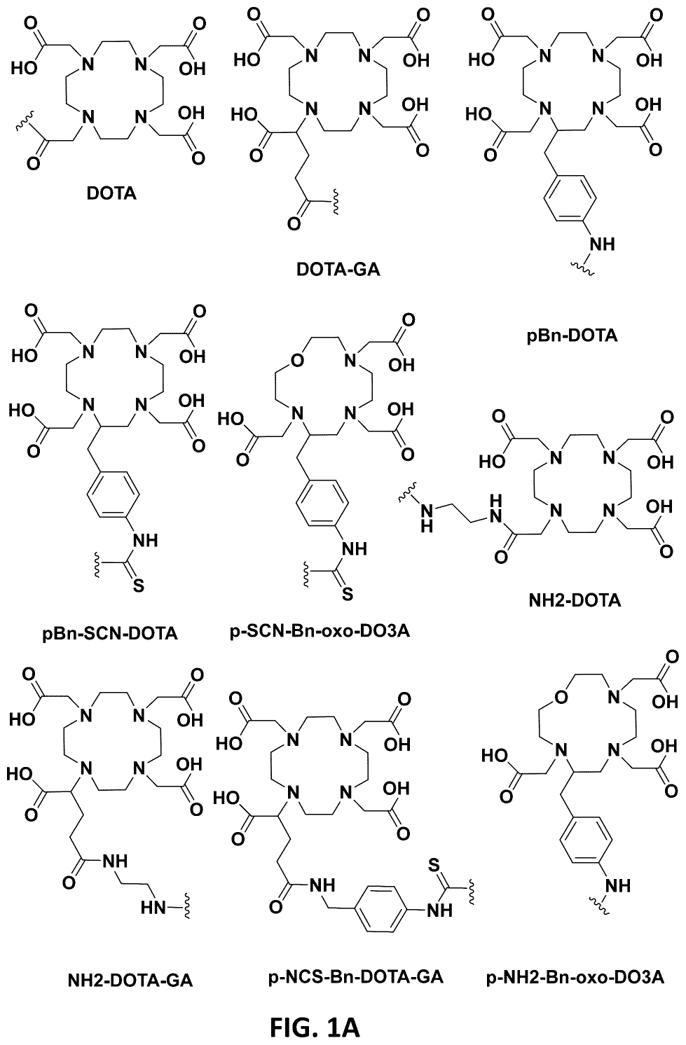

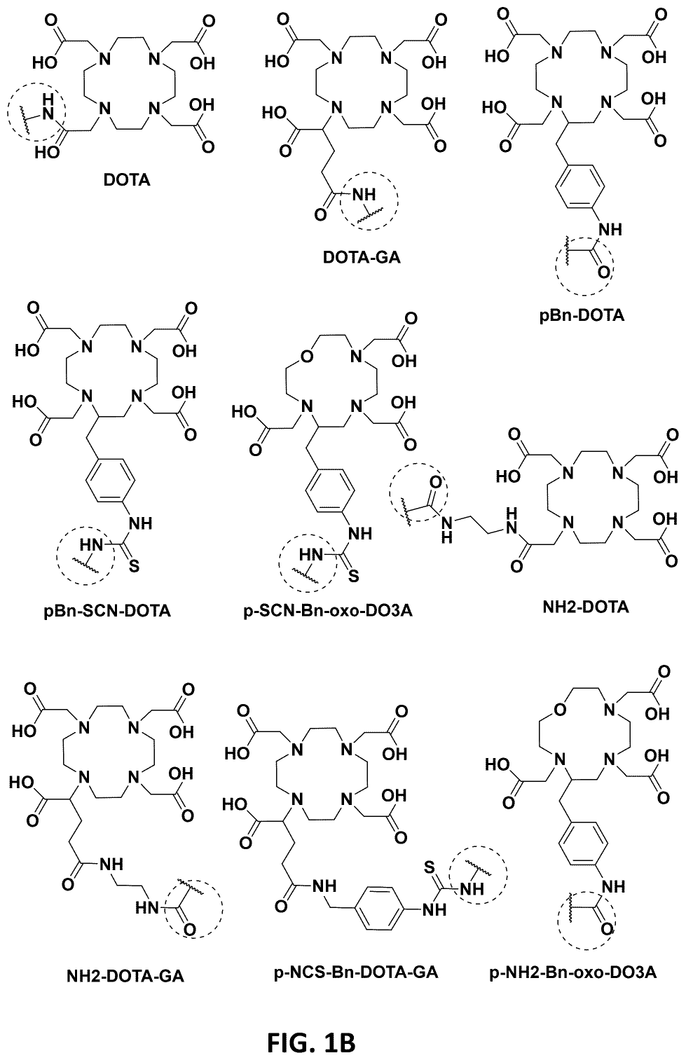

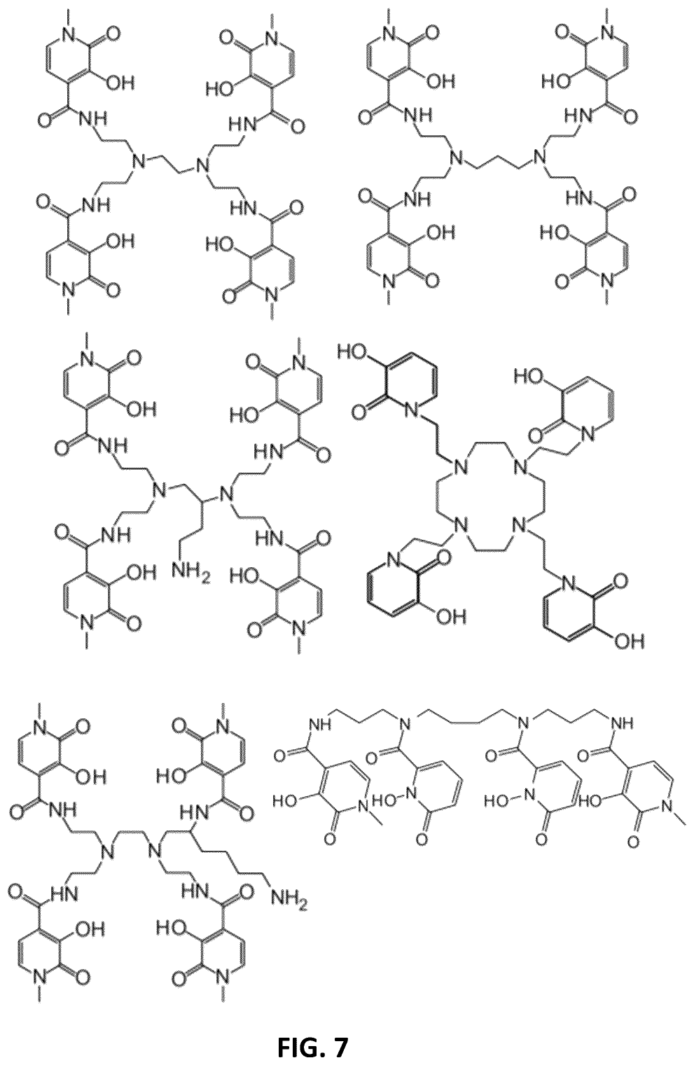

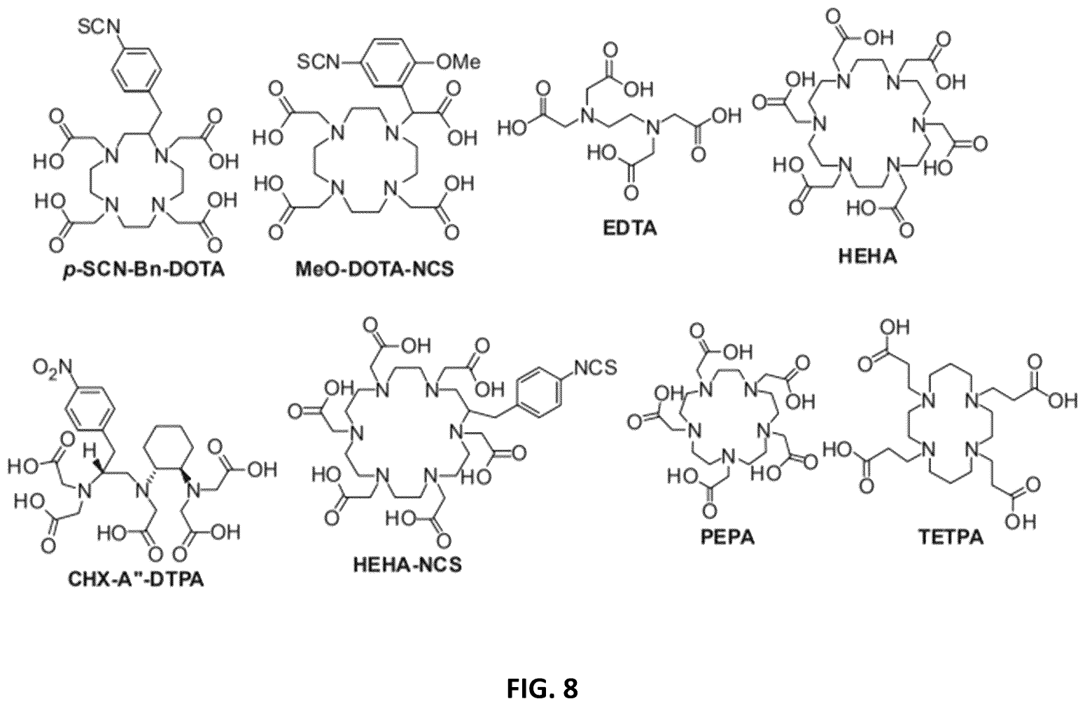

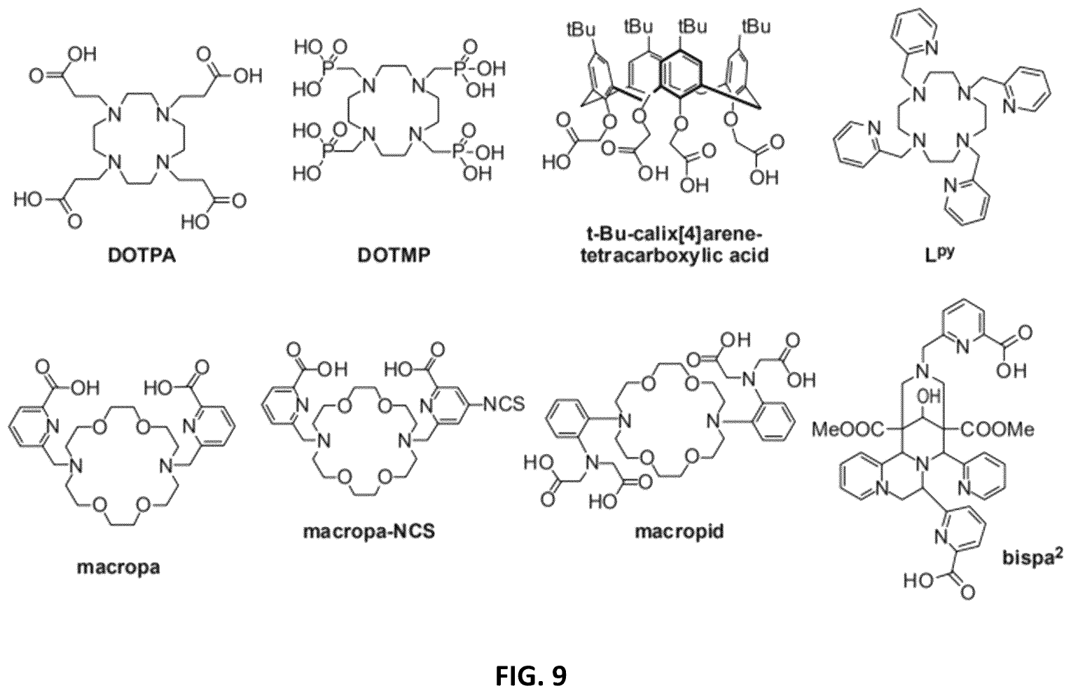

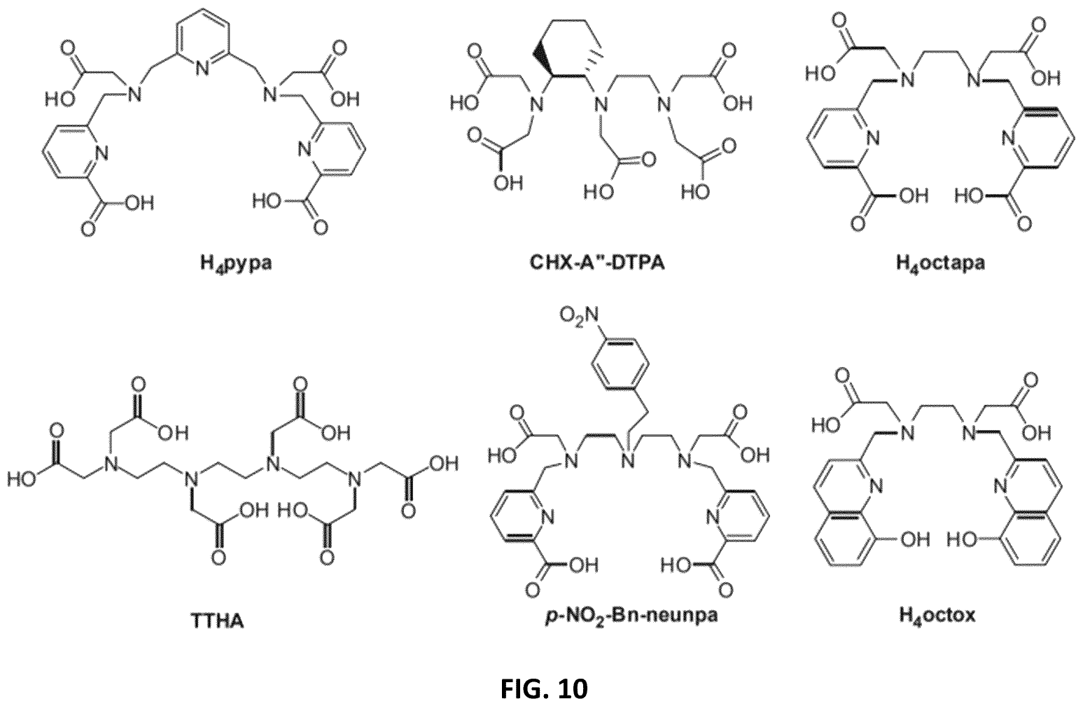

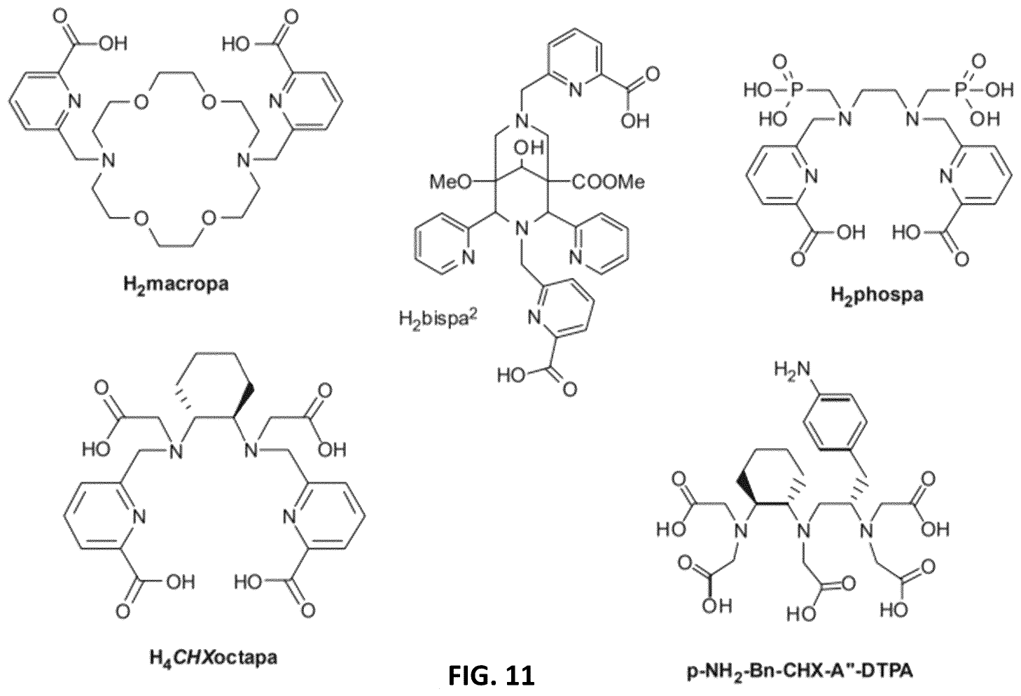

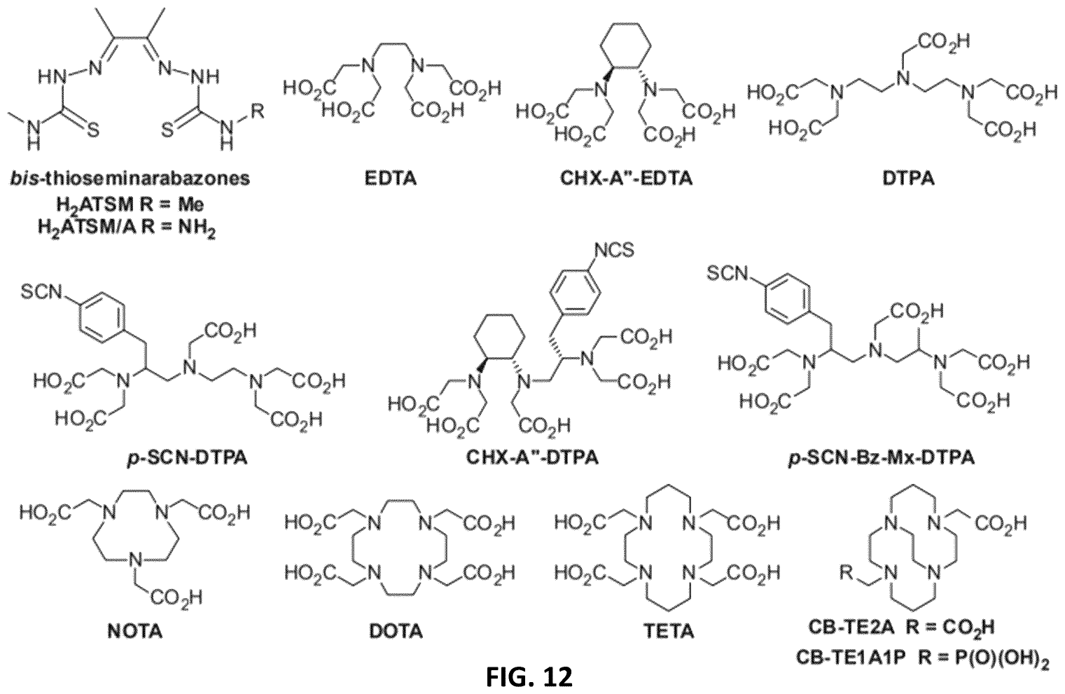

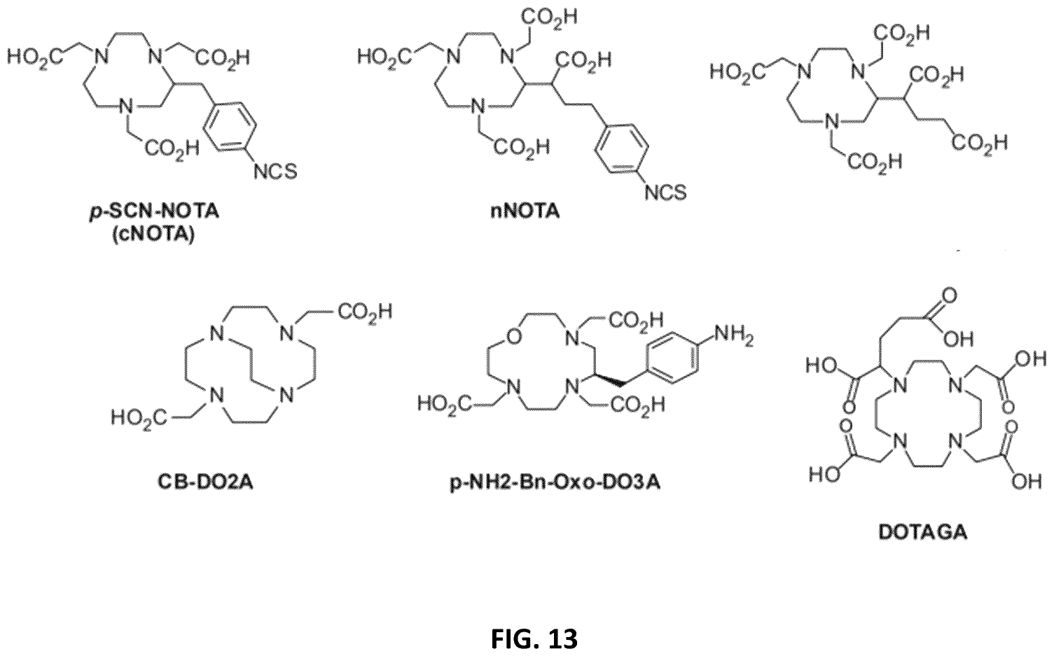

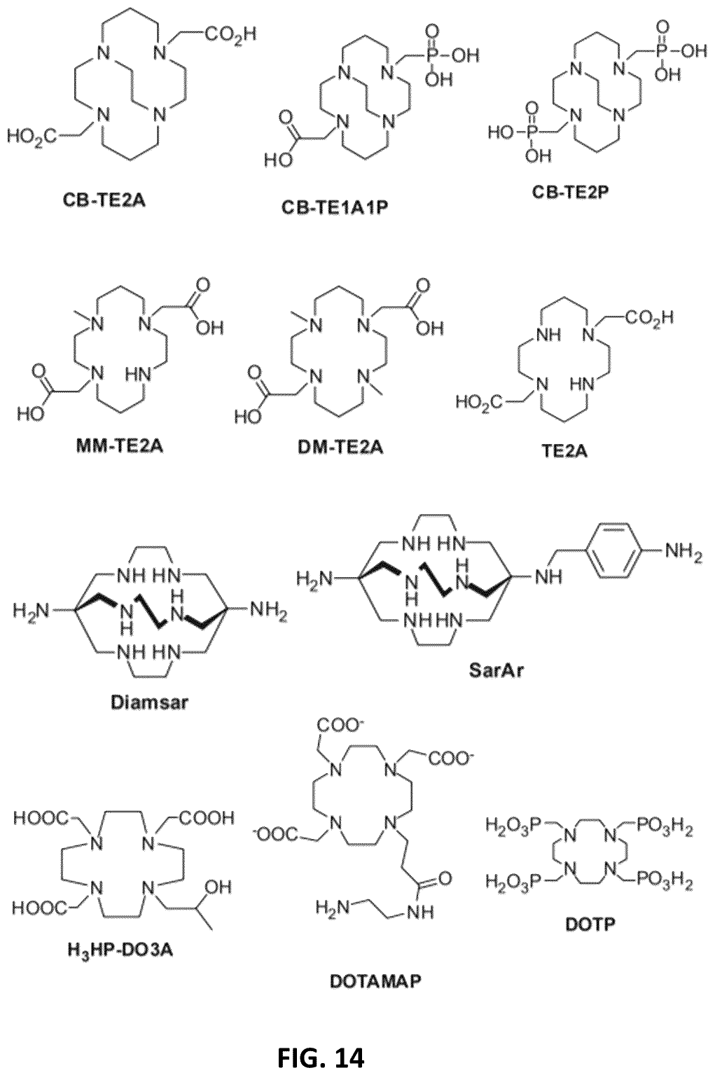

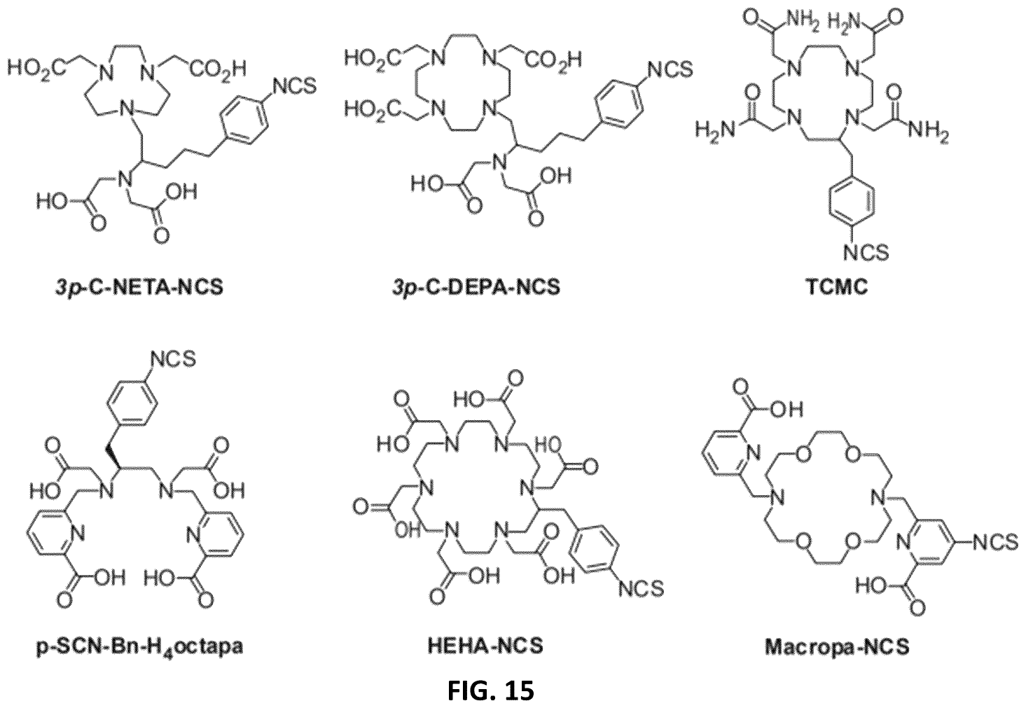

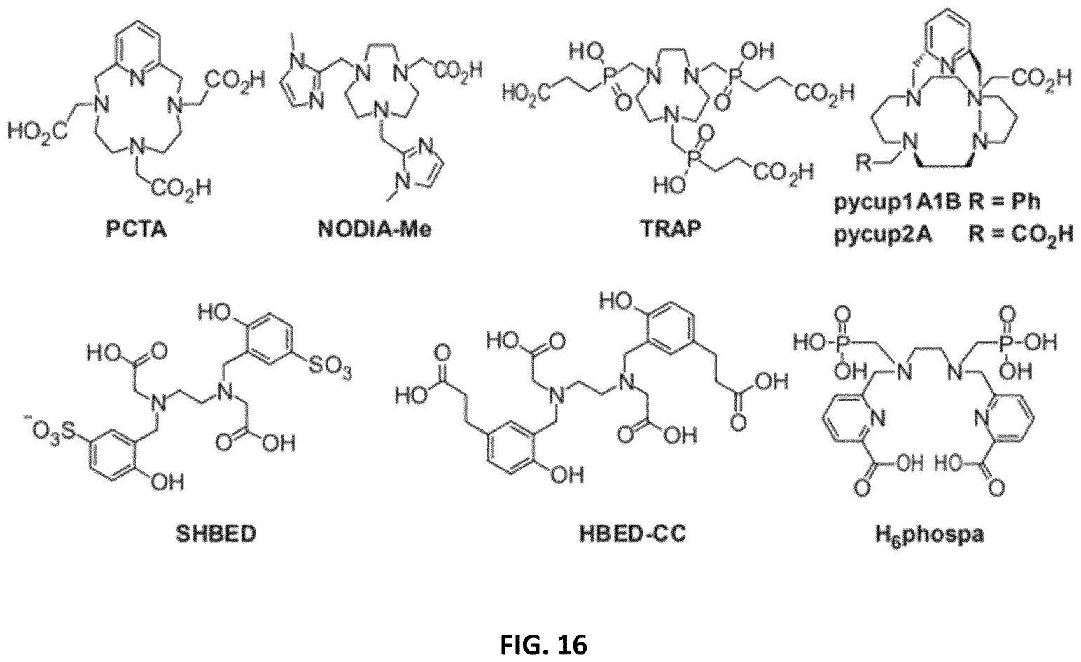

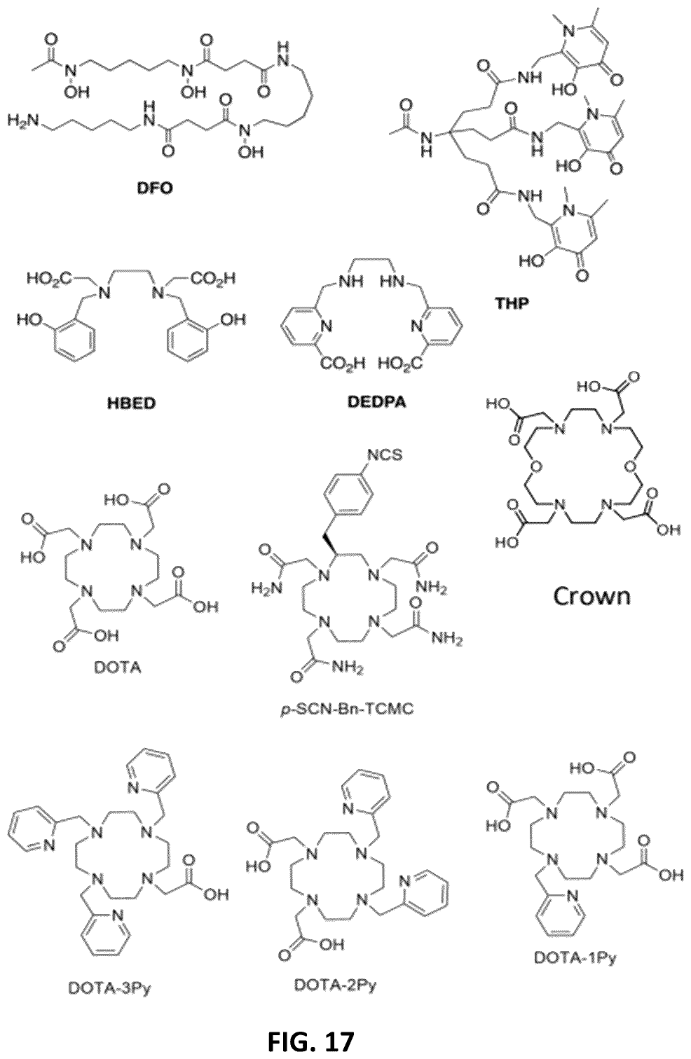

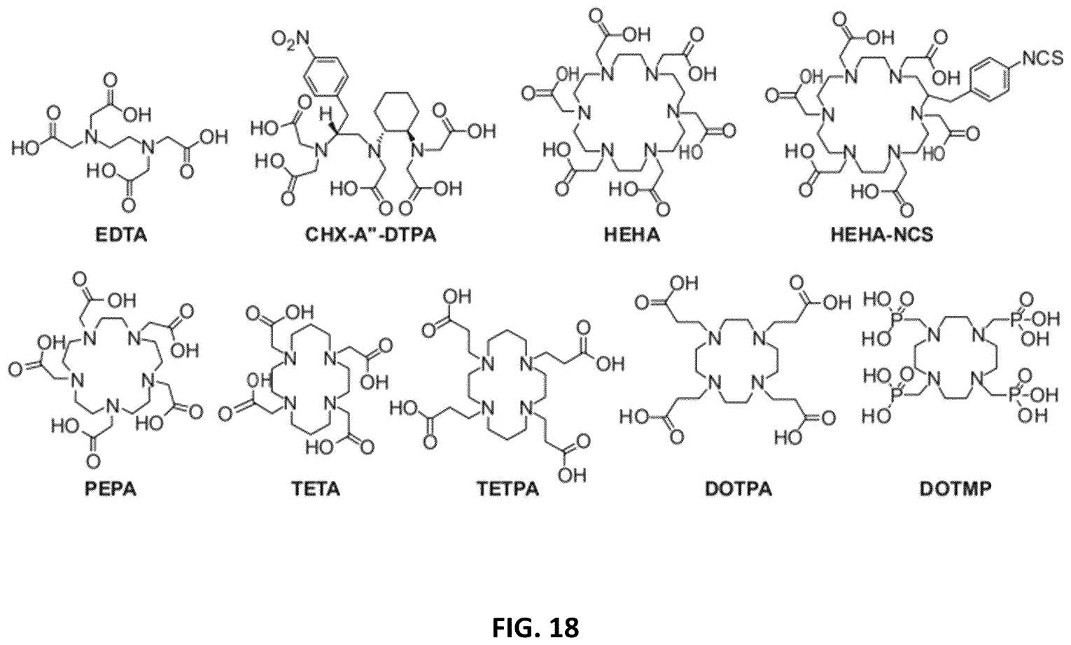

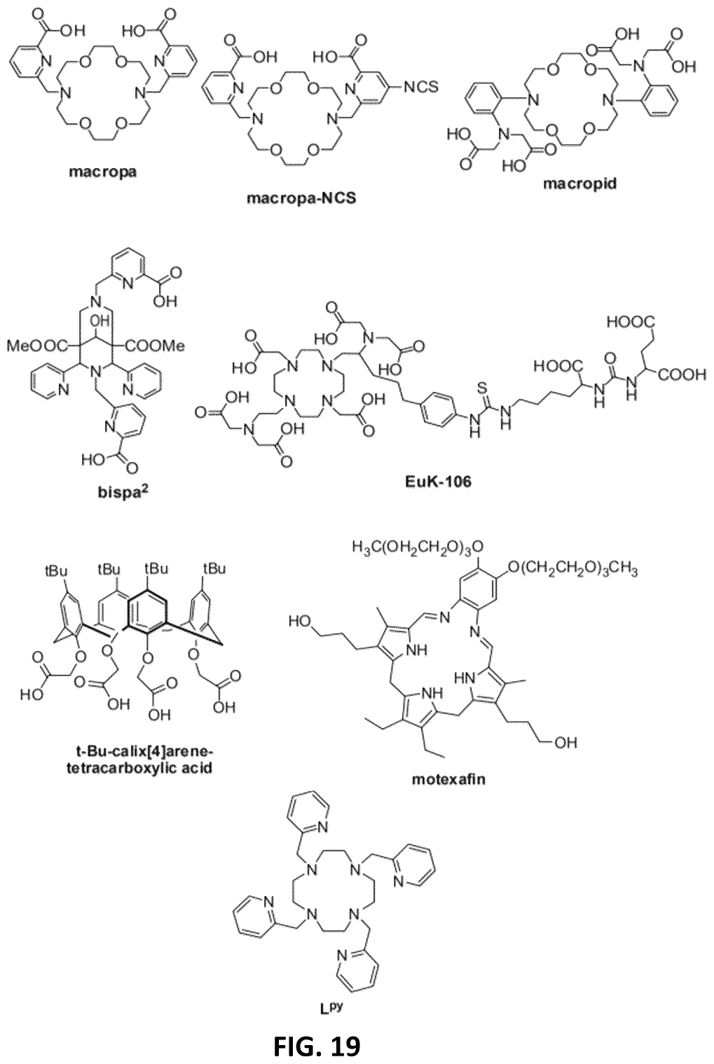

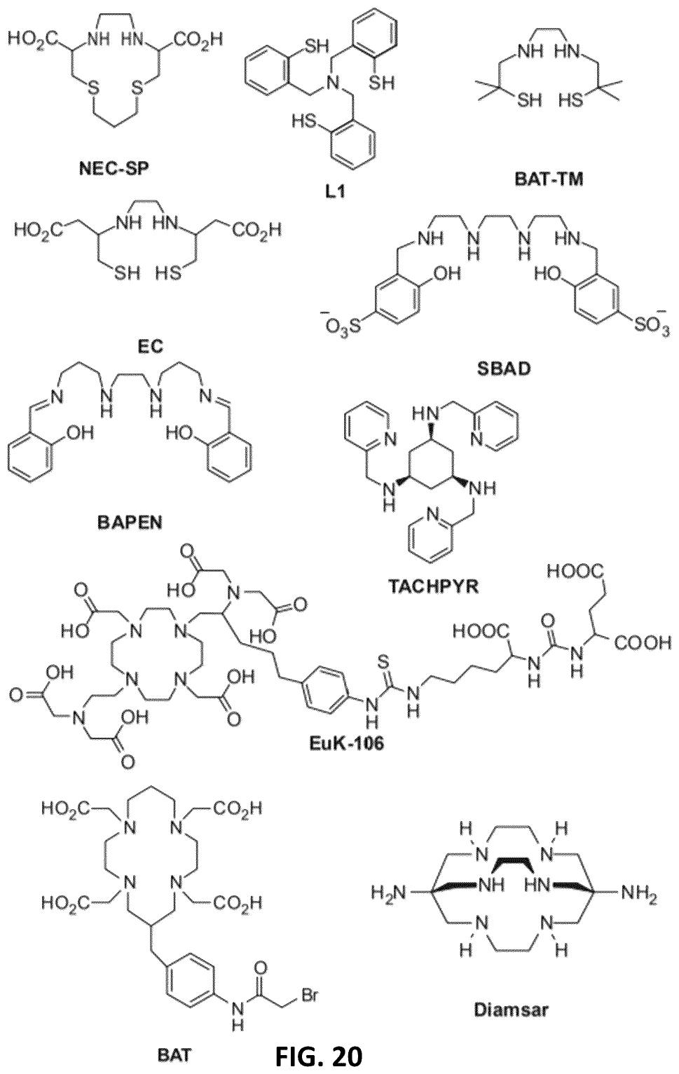

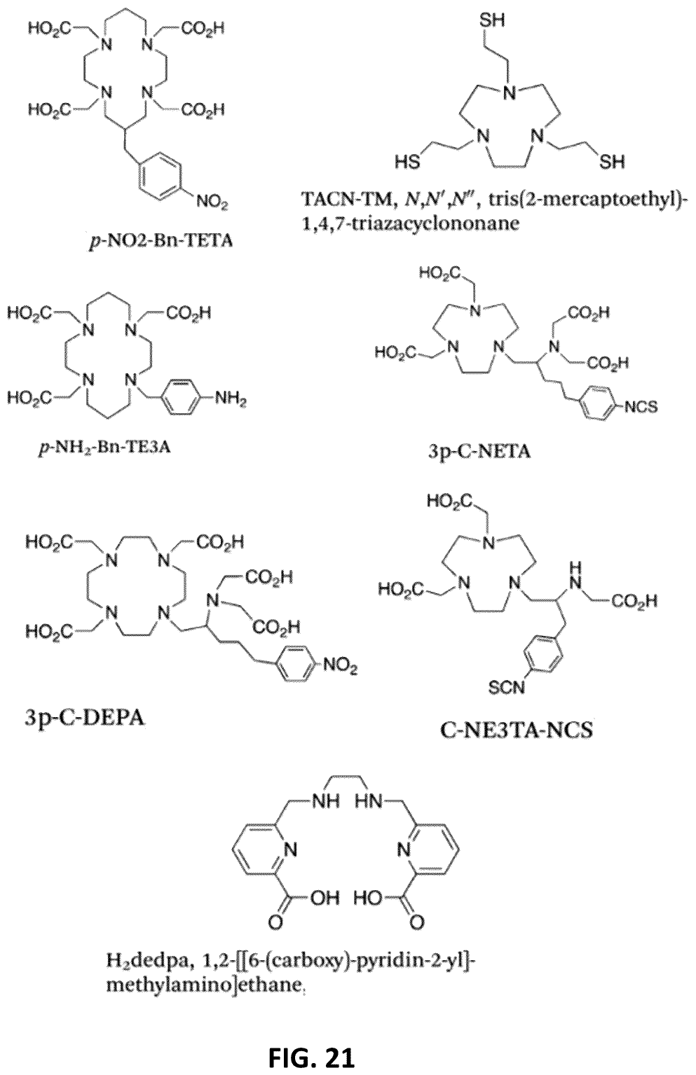

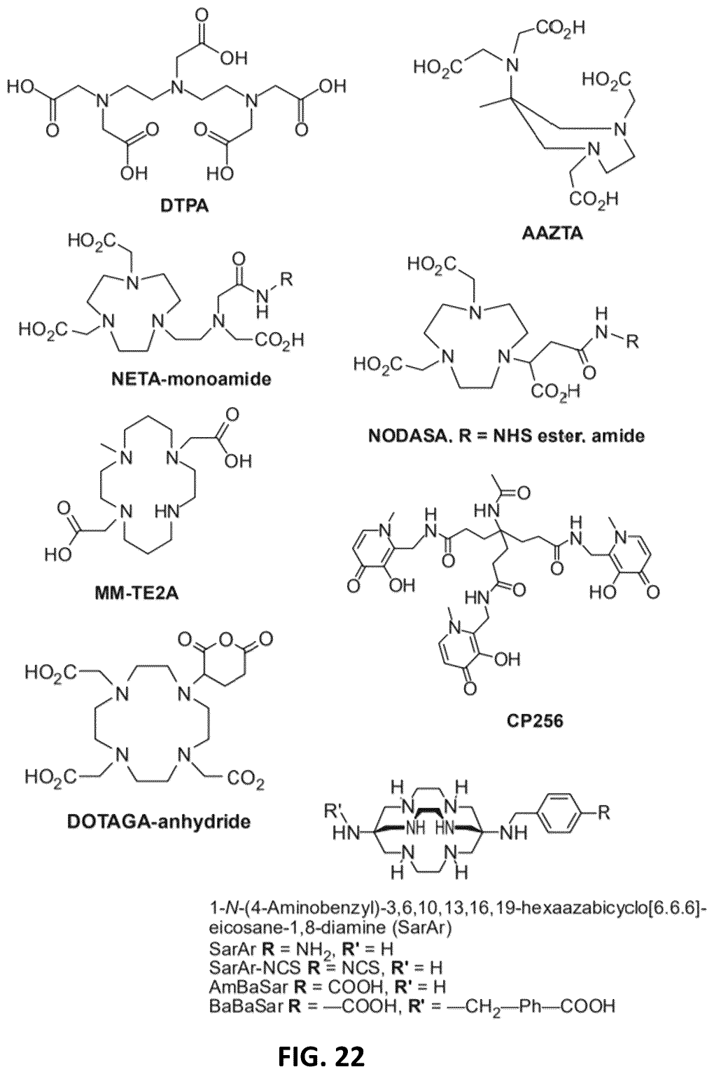

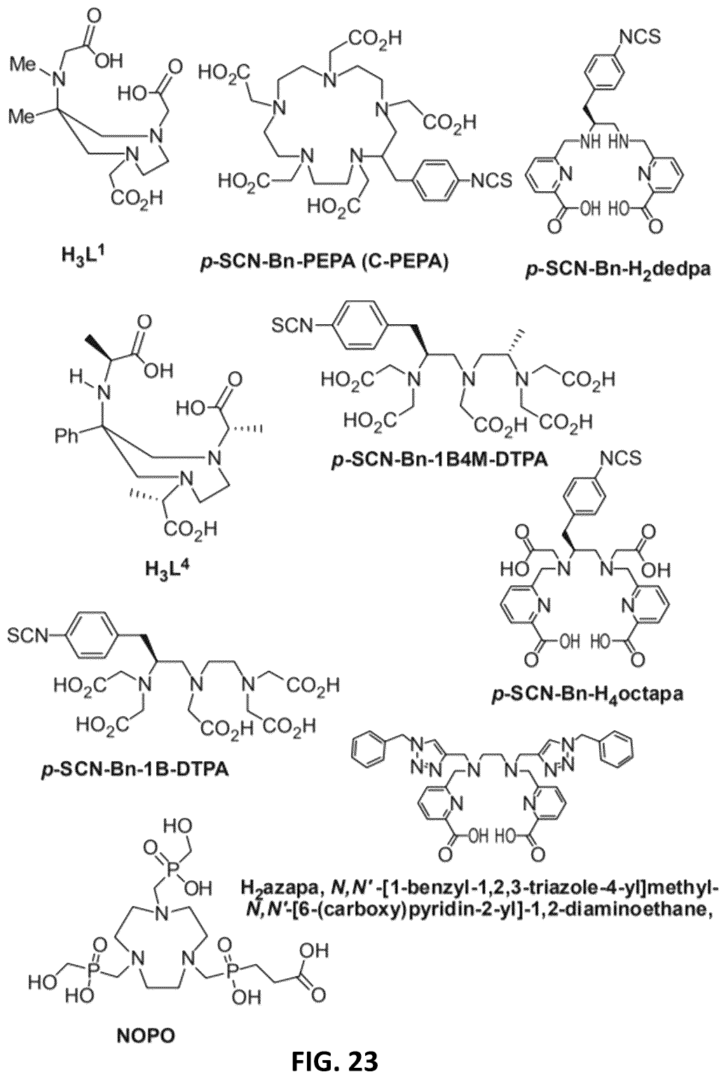

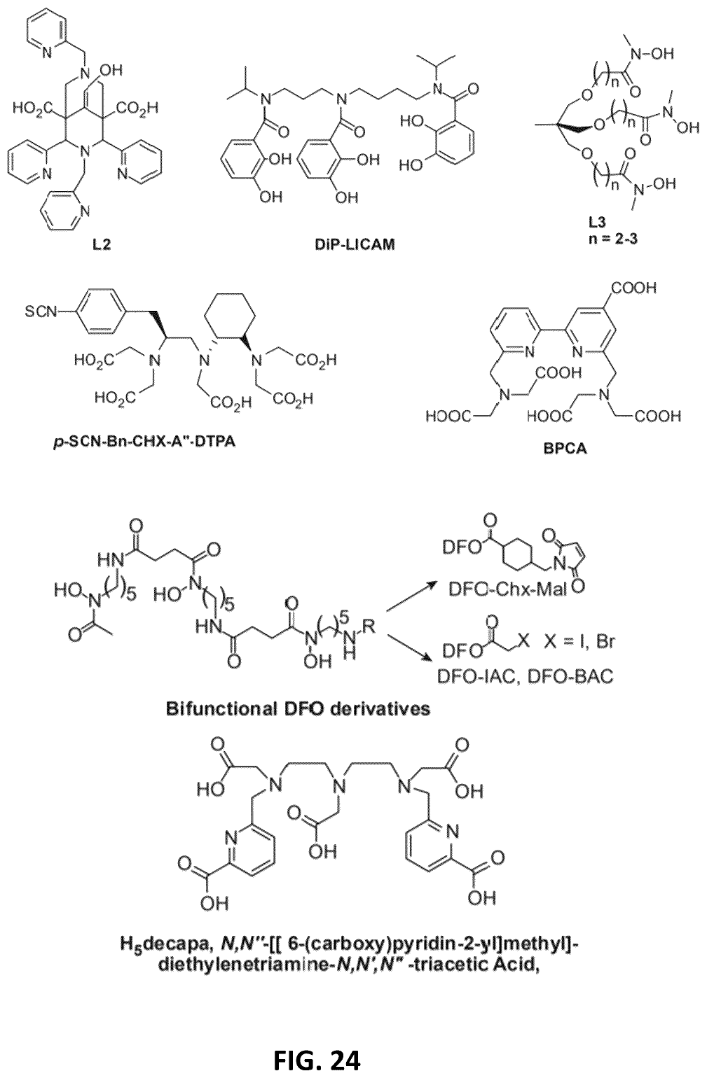

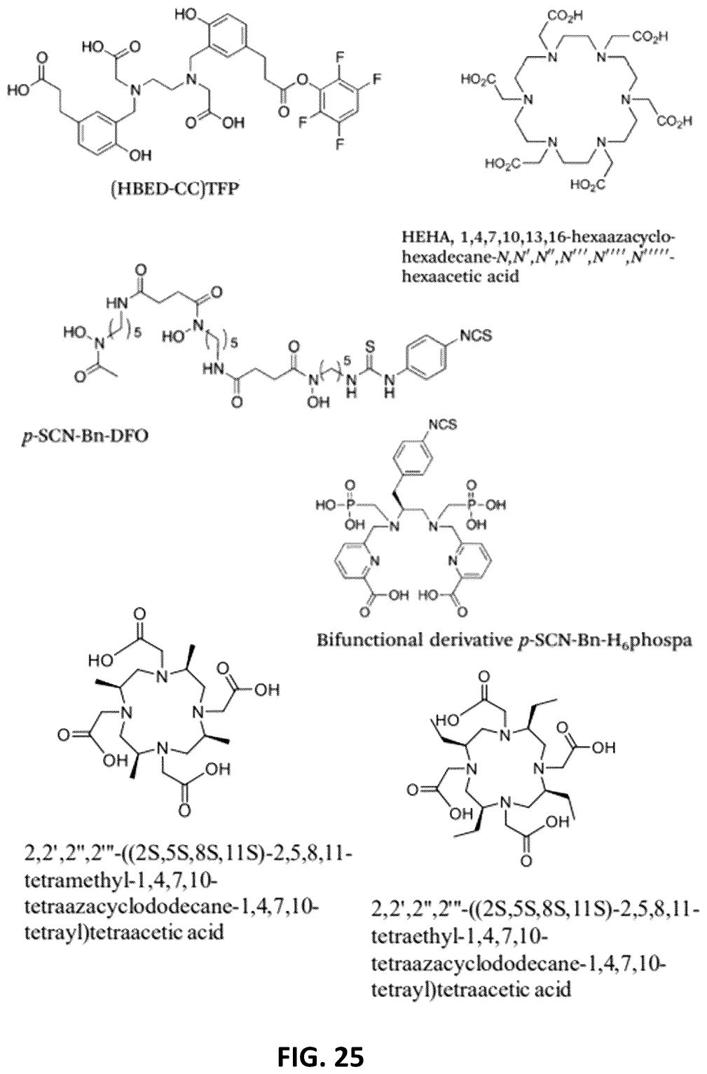

Conversely, various features of the disclosure, which are, for brevity, described in the context of a single embodiment, can also be provided separately or in any suitable sub combination. For example, a conjugate of this disclosure can comprise any peptide ligand described herein (e.g., a peptide ligand of Formula (I), (I′), (I″), (II), or Table 1), any metal chelator described herein (e.g., a metal chelator selected from FIGS. 1A, 1B, 2A, 2B, 3A, 3B, 4A, 4B, 5A, 5B, and 6-25), optionally a linker described herein (e.g., a linker of Formula (V-1), (V-1a), or (V-1b)), and optionally a radionuclide described herein (e.g., a radionuclide of Table 4). For another example, a peptide of Formula (I), (I′), (I″), or (II) can comprise X1 to X6 amino acids as described herein, and any combinations of the embodiments of amino acids are encompassed by this disclosure (even though, in some cases, they are described in the context of separate embodiments). As another example, the disclosure also encompass any conjugates that comprises a structure of Table 3 and a radionuclide of Table 4.

Unless special definitions are given, the terminology used in relation to analytical chemistry, synthetic organic chemistry, and medical chemistry and pharmaceutical chemistry described in the present specification, as well as their procedures and techniques, are well known and commonly used in the field of the present art. Standard techniques may be used for chemical synthesis and chemical analysis. Those defined from among such techniques and procedures can be found in, for example, “K. J. Jensen, P. T. Shelton, S. L. Pedersen, Peptide Synthesis and Applications, 2nd Edition, Springer, 2013” and the like, and these are incorporated into the present specification by reference for all purposes. All patents, applications, published applications, and other publications, and other data referred to throughout the entire disclosure, when permitted, are incorporated into the present specification by reference.

### II. Peptides and Radiopharmaceutical Conjugates

Targeted Radiopharmaceuticals (TRP) are a new generation of nuclear medicine for cancer treatment or diagnosis. A TRP can selectively deliver high concentrations of radionuclide-containing molecules to the target cells such as a tumor, and no or very low concentrations to the undesired cells present in normal, healthy tissues. The process can be achieved by engineering the drug molecule with the high-affinity binder (e.g., targeting ligands) and linking it to the radioactive isotope. The biological targets of these binders are highly expressed on tumor cells and have low or no expression in healthy tissues and organs. When the radioisotope decays, it emits highly energic ionizing radiation in form of alpha, beta, and/or gamma particles. The released energy at the target sites can cause damage or death of the target tissues or be visualized by imaging scanner to achieve therapeutic or diagnostic purposes.

Provided herein are radiopharmaceutical conjugates, or a pharmaceutically acceptable salt thereof, that have avidity for the somatostatin receptor and pharmaceutical compositions comprising the conjugates. Also provided herein are peptides, or a pharmaceutically acceptable salt thereof, that have avidity for the somatostatin receptor and pharmaceutical compositions comprising the peptides. The conjugates, peptides, and compositions can be useful for treating cancer. The conjugates, peptides, and compositions can also be useful in imaging and disease diagnosis.

In one aspect, described herein is a peptide having avidity for a somatostatin receptor (SSTR), wherein the peptide comprises a structure of Formula (I) or a salt thereof,

X1-X2-X3-X4-X5-X6 Formula (I)
wherein,
X1 is any amino acid;
X2 is any amino acid;
X3 is a non-natural, aromatic amino acid;
X4 is a non-aromatic amino acid having a side chain comprising an amine;
X5 is any amino acid; and
X6 is any amino acid.

In one aspect, described herein is a peptide having avidity for a somatostatin receptor (SSTR), wherein the peptide comprises a structure of Formula (I′) or a salt thereof,

X1-X2-X3-X4-X5-X6 Formula (I′)
wherein,
X1 is any amino acid;
X2 is any amino acid;
X3 is an aromatic amino acid;
X4 is a non-aromatic amino acid having a side chain comprising an amine;
X5 is any amino acid; and
X6 is any amino acid.
   In some embodiments, X3 is a Trp.

In one aspect, described herein is a peptide having avidity for a somatostatin receptor (SSTR), wherein the peptide comprises a structure of Formula (I″) or a salt thereof,

X1-X2-X3-X4-X5-X6 Formula (I″)
wherein,
X1 is any amino acid;
X2 is any amino acid;
X3 is non-natural, aromatic amino acid;
X4 is a Lys;
X5 is any amino acid; and
X6 is any amino acid.

In some embodiments of Formula (I), (I′), or (I″), the SSTR is a human SSTR. In some embodiments, the SSTR is somatostatin receptor type 1 (SSTR1), somatostatin receptor type 2 (SSTR2), somatostatin receptor type 3 (SSTR3), somatostatin receptor type 4 (SSTR4), and/or somatostatin receptor type 5 (SSTR5). In some embodiments, the SSTR is SSTR2. In some embodiments, the peptide is cyclic or acyclic. In some embodiments, the peptide is cyclic. In some embodiments, the peptide is monocyclic and has a structure of Formula (II), or a pharmaceutically acceptable salt thereof:

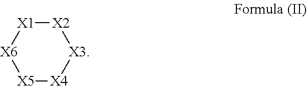

In some embodiments, the peptide of Formula (I), (I′), (I″), or Formula (II), or a pharmaceutically acceptable salt thereof, is a conjugate having avidity for a somatostatin receptor. In some embodiments, the conjugate further comprises a metal chelator covalently connected to the peptide. In some embodiments, the metal chelator is configured to bind with a radionuclide. In some embodiments, the metal chelator is covalently connected to the monocyclic peptide through a linker. In some embodiments, the conjugate has a structure of Formula (III), or a pharmaceutically acceptable salt thereof:

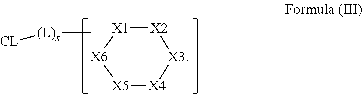

wherein,
L is a linker;
s is 0 or 1; and
CL is a metal chelator.

In some embodiments of Formula (III), CL-(L)ₛ- is attached to the monocyclic peptide at any suitable position. In some embodiments, the conjugate has the structure of Formula (IV1), (IV2), (IV3), (IV4), (IV5), or (IV6):

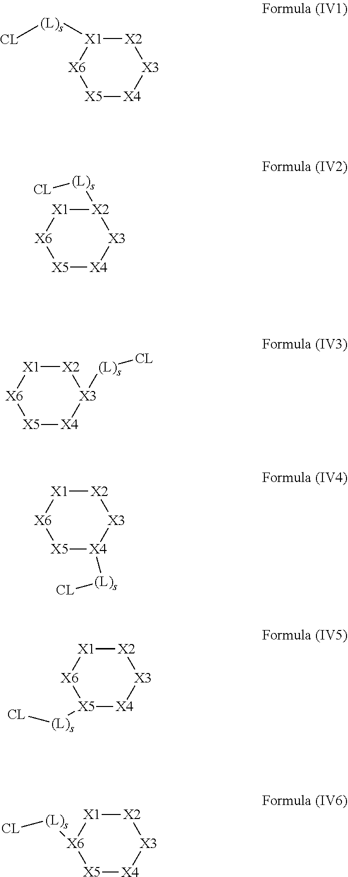

wherein,
L is a linker;
s is 0 or 1; and
CL is a metal chelator.

In some embodiments, CL-(L)ₛ- is attached to the monocyclic peptide at X1, X2, or X6. In some embodiments, the conjugate has the structure of Formula (IV1), (IV2), or (IV6):

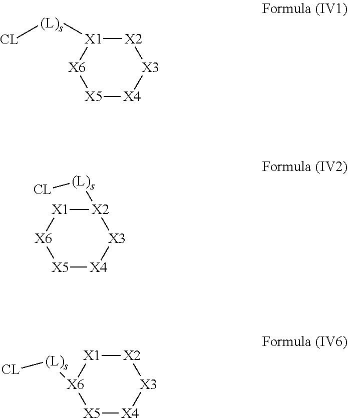

wherein,
L is a linker;
s is 0 or 1; and
CL is a metal chelator.

In some embodiments, the conjugate has the structure of Formula (IV1)

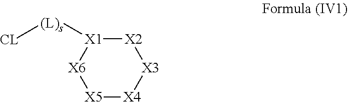

In some embodiments, the conjugate has the structure of Formula (IV2)

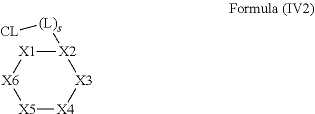

In some embodiments, the conjugate has the structure of Formula (IV6)

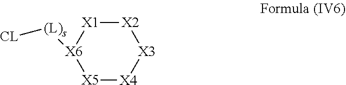

In some embodiments, s is 0 and the metal chelator is attached directly to the monocyclic peptide (e.g., the linker is a bond). In some embodiments, s is 1 and the metal chelator is attached to the monocyclic peptide through the linker L.

In some embodiments, a conjugate of Formula (III), (IV1), (IV2), (IV3), (IV4), (IV5), or (IV6), or a pharmaceutically acceptable salt thereof, comprises a radionuclide bound to the metal chelator.

In some embodiments of Formula (I), (I′), (I″), (II), (III), (IV1), (IV2), (IV3), (IV4), (IV5), or (IV6), or a pharmaceutically acceptable salt thereof, X1 is an N-alkylated amino acid. In some embodiments, X1 is an N-methylated amino acid. In some embodiments, X1 is any amino acid comprising a polar side chain. In some embodiments, X1 is an L-amino acid. In some embodiments, X1 is an N-methylated L-amino acid comprising a polar side chain. In some embodiments, X1 is Cys, Lys, Ala, Glu, Asp, Ser, Pro, or a derivative thereof. In some embodiments, X1 is Cys, Lys, Ala, Glu, Asp, Ser, or a derivative thereof. In some embodiments, X1 is Cys or a derivative thereof. In some embodiments, X1 is Lys or a derivative thereof. In some embodiments, X1 is Ala or a derivative thereof. In some embodiments, X1 is Glu or a derivative thereof. In some embodiments, X1 is Asp or a derivative thereof. In some embodiments, X1 is Ser or a derivative thereof. In some embodiments, X1 is Gly. In some embodiments, X1 is Pro or a derivative thereof. In some embodiments, X1 is not Pro or a derivative thereof. In some embodiments, X1 is Pro, Hyp, Cha4N, Chg4N, NMe-Cha4N, NMe-Chg4N, NMe-Dap, NMe-Dab, NMe-Orn, NMe-Lys, NMe-Azidolysine, NMe-hLys, 4-oxa NMe-Lys, NMe-Ala, NMe-Amp, NMe-Nle, NMe-propargyl glycine, NMe-propargyl alanine, NMe-Asp, NMe-Cys, NMe-Hcy, NMe-hHcy, NMe-Glu, NMe-hGlu, NMe-Hse, or NMe-Hse (Se). In some embodiments, X1 is Cha4N, Chg4N, NMe-Cha4N, NMe-Chg4N, NMe-Dap, NMe-Dab, NMe-Orn, NMe-Lys, NMe-Azidolysine, NMe-hLys, 4-oxa NMe-Lys, NMe-Ala, NMe-Amp, NMe-Nle, NMe-propargyl glycine, NMe-propargyl alanine, NMe-Asp, NMe-Cys, NMe-Hcy, NMe-hHcy, NMe-Glu, NMe-hGlu, NMe-Hse, or NMe-Hse (Se). In some embodiments, X1 is optionally substituted Pro. In some embodiments, X1 is optionally substituted Hyp. In some embodiments, X1 is optionally substituted Cha4N. In some embodiments, X1 is optionally substituted Chg4N. In some embodiments, X1 is optionally substituted NMe-Cha4N. In some embodiments, X1 is optionally substituted NMe-Chg4N. In some embodiments, X1 is optionally substituted NMe-Dap. In some embodiments, X1 is optionally substituted NMe-Dab. In some embodiments, X1 is optionally substituted NMe-Orn. In some embodiments, X1 is optionally substituted NMe-Lys. In some embodiments, X1 is optionally substituted NMe-Azidolysine. In some embodiments, X1 is optionally substituted NMe-hLys. In some embodiments, X1 is optionally substituted 4-oxa NMe-Lys, In some embodiments, X1 is optionally substituted NMe-Ala. In some embodiments, X1 is optionally substituted NMe-Amp. In some embodiments, X1 is optionally substituted NMe-Nle. In some embodiments, X1 is optionally substituted NMe-propargyl glycine. In some embodiments, X1 is optionally substituted NMe-propargyl alanine. In some embodiments, X1 is optionally substituted NMe-Asp. In some embodiments, X1 is optionally substituted NMe-Cys. In some embodiments, X1 is optionally substituted NMe-Hcy. In some embodiments, X1 is optionally substituted NMe-hHcy. In some embodiments, X1 is optionally substituted NMe-Glu. In some embodiments, X1 is optionally substituted NMe-hGlu. In some embodiments, X1 is optionally substituted NMe-Hse. In some embodiments, X1 is optionally substituted NMe-Hse (Se). In some embodiments, X1 is Cys, Lys, or a derivative thereof. In some embodiments, X1 is Cha4N, Chg4N, NMe-Cha4N, NMe-Chg4N, NMe-Dap, NMe-Dab, NMe-Orn, NMe-Lys, NMe-hLys, 4-oxa NMe-Lys, NMe-Azidolysine, NMe-Cys, NMe-Hcy, NMe-hHcy, or NMe-Hse (Se). In some embodiments, X1 is Cys or a derivative thereof. In some embodiments, X1 is NMe-Cys, NMe-Hcy, NMe-Hse (Se), or NMe-hHcy. In some embodiments, X1 is Lys or a derivative thereof. In some embodiments, X1 is NMe-Dap, NMe-Dab, NMe-Lys, NMe-hLys, or NMe-Orn. In some embodiments, X1 is Ala or a derivative thereof. In some embodiments, X1 is NMe-Ala, NMe-Nle, or NMe-propargyl glycine. In some embodiments, X1 is a peptoid. In some embodiments, X1 is Nlys. In some embodiments, the metal chelator is attached to X1 and the linker is a bond. In some embodiments, the metal chelator is attached to X1 through a linker.

In some embodiments of Formula (I), (I′), or (I″), or a pharmaceutically acceptable salt thereof, X1 has a structure of:

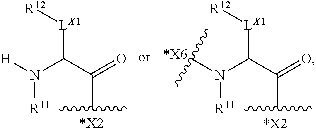

wherein,
R¹¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
R¹² is C₁-C₆alkyl, C₁-C₆hydroxyalkyl, C₁-C₆aminoalkyl, C₁-C₆heteroalkyl, C₃-C₆cycloalkyl, or 3- to 6-membered heterocycloalkyl, wherein each of the alkyl, heteroalkyl, cycloalkyl, and heterocycloalkyl is optionally substituted with one or more R^{12a};
L^{X1} is a bond, —O—, —S—, —NR¹³—, C₁-C₆alkylene, or C₁-C₆heteroalkylene, wherein the alkylene or heteroalkylene is optionally substituted with one or more R^{X1a}, or
R¹¹ and L^{X1}-R¹² are taken together with the intervening atoms to form a 5- to 6-membered heterocycloalkyl, which is optionally substituted with one or more R^{12a}.
each R^{12a} is independently halogen, C₁-C₆alkyl C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR₃, —SR^{a}, —NR^{c}R^{d}, —SeR^{a}, —S(═O)R^{a}, —S(═O)₂R², —SF₅, —S(═O)₂NR^{c}R^{d}, —S(═O)(═NR²)R^{a}, —N═S(═O)R^{c}R^{d}, —NR^{a}S(═O)₂R^{a}, amidinyl, —NR^{a}C(═NH)(NR^{a})₂, —NR^{a}S(═O)—NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or —N(R^{a}), wherein each of the alkyl, heteroalkyl, alkenyl, alkynyl, aryl, heteroaryl, cycloalkyl, and heterocycloalkyl is optionally substituted with one or more R^{e}; or, one of R^{12a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R¹³ is hydrogen or C₁-C₆alkyl;
R^{X1a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X1a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C, —C₆alkyl, —SF₅, —S(═O)C₁-C₆alkyl, —S(═O)₂C₁-C₆alkyl, —S(═O)₂NH2, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{e} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X6 represents the point of attachment to X6; and
*X2 represents the point of attachment to X2.

In some embodiments, X1 has a structure of

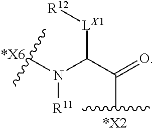

In some embodiments, X1 has a structure of

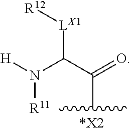

In some embodiments of Formula (I), (I′), (I″), (II), (III), (IV1), (IV2), (IV3), (IV4), (IV5), or (IV6), or a pharmaceutically acceptable salt thereof, X1 has a structure of:

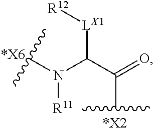

wherein,
R¹¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
R¹² is C₁-C₆alkyl, C₁-C₆hydroxyalkyl, C₁-C₆aminoalkyl, C₁-C₆heteroalkyl, C₃-C₆cycloalkyl, or 3- to 6-membered heterocycloalkyl, wherein each of the alkyl, heteroalkyl, cycloalkyl, and heterocycloalkyl is optionally substituted with one or more R^{12a},
L^{X1} is a bond, —O—, —S—, —NR¹³—, C₁-C₆alkylene, or C₁-C₆heteroalkylene, wherein the alkylene or heteroalkylene is optionally substituted with one or more R^{X1a}, or
R¹¹ and L^{X1}-R¹² are taken together with the intervening atoms to form a 5- to 6-membered heterocycloalkyl, which is optionally substituted with one or more R^{12a}.
each R^{12a} is independently halogen, C₁-C₆alkyl C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR₃, —SR^{a}, —NR^{c}R^{d}, —SeR^{a}, —S(═O)R^{a}, —S(═O) ¿R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or —N(R^{a}), wherein each of the alkyl, heteroalkyl, alkenyl, alkynyl, aryl, heteroaryl, cycloalkyl, and heterocycloalkyl is optionally substituted with one or more R^{e}; or, one of R^{12a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R¹³ is hydrogen or C₁-C₃alkyl;
R^{X1a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X1a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X6 represents the point of attachment to X6; and
*X2 represents the point of attachment to X2.

In some embodiments of Formula (I), (I′), (I″), (II), (III), (IV1), (IV2), (IV3), (IV4), (IV5), or (IV6), or a pharmaceutically acceptable salt thereof, X1 is a peptoid and has a structure of:

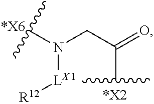

wherein,
R¹² is C₁-C₆alkyl, C₁-C₆hydroxyalkyl, C₁-C₆aminoalkyl, C₁-C₆heteroalkyl, C₃-C₆cycloalkyl, or 3- to 6-membered heterocycloalkyl, wherein each of the alkyl, heteroalkyl, cycloalkyl, and heterocycloalkyl is optionally substituted with one or more R^{12a},
L^{X1} is a bond, C₁-C₆alkylene, or C₁-C₆heteroalkylene, wherein the alkylene or heteroalkylene is optionally substituted with one or more R^{X1a}; or
each R^{12a} is independently halogen, C₁-C₆alkyl C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —SeR^{a}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R³, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR)(OR′), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, and heterocycloalkyl is optionally substituted with one or more R^{e}; or, one of R^{12a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X6 represents the point of attachment to X6; and
*X2 represents the point of attachment to X2.

In some embodiments of Formula (I), (I′), (I″), (II), (III), (IV1), (IV2), (IV3), (IV4), (IV5), or (IV6), or a pharmaceutically acceptable salt thereof, R¹² is C₁-C₆alkyl optionally substituted with one or more R^{12a}. In some embodiments, R¹² is C₁-C₆heteroalkyl optionally substituted with one or more R^{12a}. In some embodiments, R¹² is C₁-C₆hydroxyalkyl or C₁-C₆aminoalkyl, each which is optionally substituted with one or more R^{12a}. In some embodiments, R¹² is C₃-C₆cycloalky or 3- to 6-membered heterocycloalkyl, wherein each of the cycloalkyl and heterocycloalkyl is optionally substituted with one or more R^{12a}. In some embodiments, each R^{12a} is independently —OR^{a}, —SR^{a}, —NR^{c}R^{d}, —SeR^{a}, —S(═O)R^{a}, —S(═O)₂R^{a}, —S(═O)₂NR^{c}R^{d}, —NR^{a}S(═O)₂R^{a}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, or —C(═O)NR^{c}R^{d}. In some embodiments, each R^{12a} is independently —OR^{a}, —SR^{a}, —NR^{c}R^{d}, —SeR^{a}, —C(═O)R^{a}, —C(═O)OR^{a}, or —C(═O)NR^{c}R^{d}. In some embodiments, R¹² is C₁-C₆alkyl, C₃-C₆cycloalkyl, or 3- to 6-membered heterocycloalkyl, wherein each of the alkyl, cycloalkyl, and heterocycloalkyl is optionally substituted with one or more substituents independently selected from the group consisting of —OR^{a}, —SR^{a}, —NR^{c}R^{d}, —SeR^{a}, —S(═O)R^{a}, —S(═O)₂R^{a}, —S(═O)₂NR^{c}R^{d}, —NR^{a}S(═O)₂R^{a}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, and —C(═O)NR^{c}R^{d}. In some embodiments, R¹² is C₁-C₆alkyl substituted with one substituent selected from the group consisting of —OR^{a}, —SR^{a}, —NR^{c}R^{d}, —SeR^{a}, —C(═O)R^{a}, —C(═O)OR^{a}, and —C(═O)NR^{c}R^{d}. In some embodiments, R¹² is C₁-C₆alkyl. In some embodiments, L^{X1} is a bond; and R¹² is C₁-C₆alkyl substituted with one substituent selected from the group consisting of —OR^{a}, —SR^{a}, —NR^{c}R^{d}, —SeR^{a}, —C(═O)R^{a}, —C(═O)OR^{a}, and —C(═O)NR^{c}R^{d}. In some embodiments, L^{X1} is a bond; and R¹² is C₁-C₆alkyl.

In some embodiments of Formula (I), (I′), (I″), (II), (III), (IV1), (IV2), (IV3), (IV4), (IV5), or (IV6), or a pharmaceutically acceptable salt thereof, L^{X1} is a bond. In some embodiments, L^{X1} is a bond; and R¹² is C₃-C₆cycloalky or 3- to 6-membered heterocycloalkyl, wherein each of the cycloalkyl and heterocycloalkyl is optionally substituted with one or more R^{12a}. In some embodiments, L^{X1} is C₁-C₆alkylene optionally substituted with 1 or 2 substituents independently selected from R^{X1a}. In some embodiments, L^{X1} is C₁-C₆alkylene; and R¹² is C₃-C₆cycloalky or 3- to 6-membered heterocycloalkyl, wherein each of the cycloalkyl and heterocycloalkyl is optionally substituted with one or more R^{12a}. In some embodiments, L^{X1} is C₁-C₆alkylene; and R¹² is C₃-C₆cycloalky optionally substituted with one or more R^{12a}. In some embodiments, L^{X1} is C₁-C₆alkylene; and R¹² is 3- to 6-membered heterocycloalkyl optionally substituted with one or more R^{12a}. In some embodiments, X1 is a peptoid wherein L^{X1} is a bond and R¹² is C₁-C₆alkyl optionally substituted with one or more R^{12a}.

In some embodiments of Formula (I), (I′), (I″), (II), (III), (IV1), (IV2), (IV3), (IV4), (IV5), or (IV6), or a pharmaceutically acceptable salt thereof, one of R^{12a} is a CG. In some embodiments, R^{12a} is a CG. In some embodiments, the CG is an azide or a terminal alkyne.

In some embodiments of Formula (I), (I′), (I″), (II), (III), (IV1), (IV2), (IV3), (IV4), (IV5), or (IV6), or a pharmaceutically acceptable salt thereof, R¹¹ is hydrogen or C₁-C₃alkyl optionally substituted with one to three substituents independently selected from R^{f}. In some embodiments, R¹¹ is hydrogen. In some embodiments, R¹¹ is C₁-C₃alkyl. In some embodiments, R¹¹ is methyl. In some embodiments, R¹¹ and L^{X1}-R¹² are taken together with the intervening atoms to form a 5- to 6-membered heterocycloalkyl, which is optionally substituted with one or more R^{12a}. In some embodiments, R¹¹ and L^{X1}-R¹² are taken together with the intervening atoms to form a 5-membered heterocycloalkyl, which is optionally substituted with one or more R^{12a}. In some embodiments, R¹¹ and L^{X1}-R¹² are taken together with the intervening atoms to form a 6-membered heterocycloalkyl, which is optionally substituted with one or more R^{12a}. In some embodiments, R¹¹ and L^{X1}-R¹² are taken together with the intervening atoms to form a 5-membered heterocycloalkyl, which is optionally substituted with one or two substitutions selected from C₁-C₃alkyl, C₁-C₃haloalkyl, C₁-C₃hydroxyalkyl, —OH, and —OMe.

In some embodiments of Formula (I), (I′), (I″), (II), (III), (IV1), (IV2), (IV3), (IV4), (IV5), or (IV6), or a pharmaceutically acceptable salt thereof, X2 is a D-amino acid. In some embodiments, X2 is an L-amino acid. In some embodiments, X2 is an aromatic amino acid. In some embodiments, X2 is Tyr, Phe, Trp, His, Gly, Ala, or a derivative thereof. In some embodiments, X2 is Tyr or a derivative thereof. In some embodiments, X2 is Phe or a derivative thereof. In some embodiments, X2 is Trp or a derivative thereof. In some embodiments, X2 is His or a derivative thereof. In some embodiments, X2 is Gly or a derivative thereof. In some embodiments, X2 is Ala or a derivative thereof. In some embodiments, X2 is Tyr, D-Tyr, 4Pal, 3Pal, 5F-Tyr, 3,5-diF-Tyr, Phe, 3MeO-Phe, 4MeO-Phe, Tyr(Phe), (R-βMe)Phe, (S-βMe) Phe, 3,3-diPhe, D-Phg, L-DOPA, Aph(Hor), His, 2-(Aminocarbonyl)-Phe (F2CON), 3-(Aminocarbonyl)-Phe (F3CON), Ala, or Gly. In some embodiments, X2 is Tyr, D-Tyr, 4Pal, 3Pal, 5F-Tyr, 3,5-diF-Tyr, Phe, (R-βMe) Phe, (S-Me) Phe, 3,3-diPhe, D-Phg, L-DOPA, Aph(Hor), His, 2-(Aminocarbonyl)-Phe, 3-(Aminocarbonyl)-Phe, Ala, or Gly. In some embodiments, X2 is optionally substituted Tyr. In some embodiments, X2 is optionally substituted D-Tyr. In some embodiments, X2 is optionally substituted 4Pal. In some embodiments, X2 is optionally substituted 3Pal. In some embodiments, X2 is optionally substituted 5F-Tyr. In some embodiments, X2 is optionally substituted 3,5-diF-Tyr. In some embodiments, X2 is optionally substituted Phe. In some embodiments, X2 is optionally substituted (R-βMe) Phe. In some embodiments, X2 is optionally substituted (S-βMe) Phe. In some embodiments, X2 is optionally substituted 3,3-diPhe. In some embodiments, X2 is optionally substituted D-Phg. In some embodiments, X2 is optionally substituted L-DOPA. In some embodiments, X2 is optionally substituted Aph(Hor) In some embodiments, X2 is optionally substituted His. In some embodiments, X2 is optionally substituted 2-(Aminocarbonyl)-Phe. In some embodiments, X2 is optionally substituted 3-(Aminocarbonyl)-Phe. In some embodiments, X2 is optionally substituted Ala. In some embodiments, X2 is optionally substituted Gly. In some embodiments, X2 is Tyr.

In some embodiments of Formula (I), (I′), (I″), (II), (III), (IV1), (IV2), (IV3), (IV4), (IV5), or (IV6), or a pharmaceutically acceptable salt thereof, X2 has a structure of:

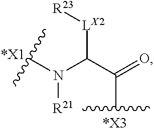

wherein:
R²¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
L^{X2} is a bond, —O—, —S—, —NR²³—, C₁-C₆alkylene, or C₁-C₆heteroalkylene, wherein the alkylene or heteroalkylene is optionally substituted with one or more R^{X2a}.
R²³ is hydrogen, C₁-C₆alkyl, C₁-C₆haloalkyl, C₁-C₆hydroxyalkyl, C₁-C₆aminoalkyl, or C₁-C₆heteroalkyl, wherein the alky and heteroalkyl is optionally substituted with one or more R^{e}; or
R²³ is

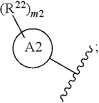

ring A2 is an aryl or heteroaryl;
each R²² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH) NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, alkenyl, alkynyl, aryl, heteroaryl, cycloalkyl, and heterocycloalkyl is optionally substituted with one or more R^{22a}, or
   two R²² are taken together to form ═O, ═S, or ═N(R^{a}); or one of R²² is a conjugation group (CG).
m2 is 0, 1, 2, 3, 4, or 5;
each R^{22a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a}) R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, and heterocycloalkyl is optionally substituted with one or more R^{e}; or, one of R^{22a} is a conjugation group (CG);
R²³ is hydrogen or C₁-C₆alkyl;
R^{X2a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X2a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl,
C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; or, one of R^{e} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X1 represents the point of attachment to X1; and
*X3 represents the point of attachment to X3.

In some embodiments of Formula (I), (I′), (I″), (II), (III), (IV1), (IV2), (IV3), (IV4), (IV5), or (IV6), or a pharmaceutically acceptable salt thereof, ring A2 is a C₆-C₁₀aryl or a 5- to 10-membered heteroaryl. In some embodiments, ring A2 is a phenyl or a 5- to 6-membered heteroaryl. In some embodiments, ring A2 is phenyl, pyridinyl, pyrimidinyl, or imidazolyl. In some embodiments, ring A2 is a C₆-C₁₀aryl. In some embodiments, ring A2 is a phenyl. In some embodiments, ring A2 is a 5- to 10-membered heteroaryl. In some embodiments, ring A2 is a 5- to 6-membered heteroaryl. In some embodiments, ring A2 is pyridinyl, pyrimidinyl, or imidazolyl. In some embodiments, ring A2 is pyridinyl or pyrimidinyl. In some embodiments, ring A2 is imidazolyl.

In some embodiments of Formula (I), (I′), (I″), (II), (III), (IV1), (IV2), (IV3), (IV4), (IV5), or (IV6), or a pharmaceutically acceptable salt thereof, each R²² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, CN, —NO₂, —OR^{a}, —SR^{a}, —NR^{c}R^{d}, —NR^{a}C(═NH) NR^{c}R^{d}, —NR^{a}S(═O)₂NR^{c}R^{d}, —C(═O)R^{a}, —C(═O)OR^{a}, —OC(═O)R^{a}, —NR^{a}C(═O)R^{a}, —NR^{a}C(═O)OR^{a}, —C(═O)NR^{c}R^{d}, C₆-C₁₀aryl, 5- to 10-membered heteroaryl, C₃-C₆cycloalkyl, or 5- to 6-membered heterocycloalkyl, wherein each of the alkyl, heteroalkyl, aryl, heteroaryl, cycloalkyl, and heterocycloalkyl is optionally substituted with one or more R^{22a}. In some embodiments, each R²² is independently C₁-C₃alkyl, —OR^{a}, —SR^{a}, —NR^{c}R^{d}, halogen —C(═O)R^{a}, —C(═O)OR^{a}, —C(═O)NR^{c}R^{d}, or phenyl. In some embodiments, one of R^{22a} is a CG. In some embodiments, the CG is an azide or a terminal alkyne. In some embodiments, the CG is an azide. In some embodiments, the CG is a terminal alkyne.

In some embodiments of Formula (I), (I′), (I″), (II), (III), (IV1), (IV2), (IV3), (IV4), (IV5), or (IV6), or a pharmaceutically acceptable salt thereof, R²¹ is hydrogen or C₁-C₃alkyl optionally substituted with one to three substituents independently selected from R^{f}. In some embodiments, R²¹ is hydrogen. In some embodiments, R²¹ is C₁-C₃alkyl. In some embodiments, R²¹ is methyl.

In some embodiments of Formula (I), (I′), (I″), (II), (III), (IV1), (IV2), (IV3), (IV4), (IV5), or (IV6), or a pharmaceutically acceptable salt thereof, L^{X2} is C₁-C₃alkylene, optionally substituted with one or more R^{X2a}. In some embodiments, L^{X2} is C₁-C₃alkylene, optionally substituted with one to three substituents selected from C₁-C₃alkyl (e.g., methyl). In some embodiments, L^{X2} is —CH₂—, —CH(CH₃)—, or —CH(phenyl)-. In some embodiments, L^{X2} is —CH₂— or —CH(CH₃)—. In some embodiments, L^{X2} is —CH₂—. In some embodiments, L^{X2} is —CH(CH₃)—. In some embodiments, L^{X2} is —CH(phenyl)-.

In some embodiments of Formula (I), (I′), (I″), (II), (III), (IV1), (IV2), (IV3), (IV4), (IV5), or (IV6), or a pharmaceutically acceptable salt thereof, m2 is 0, 1, or 2. In some embodiments, m2 is 1 or 2. In some embodiments, m2 is 0 or 1. In some embodiments, m2 is 0. In some embodiments, m2 is 1. In some embodiments, m2 is 2.

In some embodiments of Formula (I), (I′), (I″), (II), (III), (IV1), (IV2), (IV3), (IV4), (IV5), or (IV6), or a pharmaceutically acceptable salt thereof, X3 is a non-natural amino acid comprising an optionally substituted N-containing 5- to 10-membered heteroaryl. In some embodiments, X3 is a non-natural amino acid comprising an optionally substituted pyridinyl, an optionally substituted indolyl, an optionally substituted azaindolyl, an optionally substituted indazolyl, an optionally substituted benzimidazolyl, an optionally substituted pyrimidazolyl, an optionally substituted pyrazolo[1,5-a]pyridinyl, an optionally substituted quinolinyl, or an optionally substituted isoquinolinyl. In some embodiments, X3 is a Trp derivative. In some embodiments, X3 is D-Trp, NMe-D-Trp, (S-βMe) D-Trp, (S-βMe) Trp, (βGeminal methyl) D-Trp, (βGeminal methyl) Trp, (S-βPropyl) D-Trp, (S-βPropyl) Trp, (S-βIsopropyl) D-Trp, (S-βIsopropyl) Trp, (S-βCyclopropyl) D-Trp, (S-βCyclopropyl) Trp, (S-βIsobutyl) D-Trp, (S-βIsobutyl) Trp, (S-βSecbutyl) D-Trp, (S-βSecbutyl) Trp, (S-βNeopentyl) D-Trp, (S-βNeopentyl) Trp, (S-βPhenyl) D-Trp, (S-βPhenyl) Trp, (S-βBenzyl) D-Trp, (S-βBenzyl) Trp, (R-βMe) D-Trp, (R-βMe) Trp, (R-βPropyl) D-Trp, (R-βPropyl) Trp, (R-βIsopropyl) D-Trp, (R-βIsopropyl) Trp, (R-βCyclopropyl) D-Trp, (R-βCyclopropyl) Trp, (R-βIsobutyl) D-Trp, (R-βIsobutyl) Trp, (R-βSecbutyl) D-Trp, (R-βSecbutyl) Trp, (R-βNeopentyl) D-Trp, (R-βNeopentyl) Trp, (R-βPhenyl) D-Trp, (R-βPhenyl) Trp, (R-βBenzyl) D-Trp, (R-βBenzyl) Trp, Aza-Trp, Aza-D-Trp, D-6F-Trp, (5-Cl) D-Trp, (5-Me) D-Trp, (5-MeO) D-Trp, (6-Me) D-Trp, (7-Me) D-Trp, Bzt, D-Tpi, or D-Aph(Cbm), each of which is further optionally substituted. In some embodiments, the Aza-Trp is 2-aza-Trp, 4-aza-Trp, 5-aza-Trp, 6-aza-Trp, or 7-aza-Trp. In some embodiments, X3 is D-Trp, NMe-D-Trp, (S-βMe) D-Trp, (S-βMe) Trp, (R-βMe) D-Trp, (R-βMe) Trp, Aza-Trp, Aza-D-Trp, D-6F-Trp, or D-Aph(Cbm), each of which is further optionally substituted. In some embodiments, X3 is optionally substituted D-Trp. In some embodiments, X3 is optionally substituted NMe-D-Trp. In some embodiments, X3 is optionally substituted (S-βMe) D-Trp. In some embodiments, X3 is optionally substituted (S-βMe) Trp. In some embodiments, X3 is optionally substituted (βGeminal methyl) D-Trp. In some embodiments, X3 is optionally substituted (βGeminal methyl) Trp. In some embodiments, X3 is optionally substituted (S-βPropyl) D-Trp. In some embodiments, X3 is optionally substituted (S-βPropyl) Trp. In some embodiments, X3 is optionally substituted (S-Isopropyl) D-Trp. In some embodiments, X3 is optionally substituted (S-βIsopropyl) Trp. In some embodiments, X3 is optionally substituted (S-βCyclopropyl) D-Trp. In some embodiments, X3 is optionally substituted (S-βCyclopropyl) Trp. In some embodiments, X3 is optionally substituted (S-βIsobutyl) D-Trp. In some embodiments, X3 is optionally substituted (S-βIsobutyl) Trp. In some embodiments, X3 is optionally substituted (S-βSecbutyl) D-Trp. In some embodiments, X3 is optionally substituted (S-βSecbutyl) Trp. In some embodiments, X3 is optionally substituted (S-βNeopentyl) D-Trp. In some embodiments, X3 is optionally substituted (S-βNeopentyl) Trp. In some embodiments, X3 is optionally substituted (S-βPhenyl) D-Trp. In some embodiments, X3 is optionally substituted (S-βPhenyl) Trp. In some embodiments, X3 is optionally substituted (S-βBenzyl) D-Trp. In some embodiments, X3 is optionally substituted (S-βBenzyl) Trp. In some embodiments, X3 is optionally substituted (R-βMe) D-Trp. In some embodiments, X3 is optionally substituted (R-βMe) Trp. (R-βPropyl) D-Trp. In some embodiments, X3 is optionally substituted (R-βPropyl) Trp. In some embodiments, X3 is optionally substituted (R-βIsopropyl) D-Trp. In some embodiments, X3 is optionally substituted (R-βIsopropyl) Trp. In some embodiments, X3 is optionally substituted (R-βCyclopropyl) D-Trp. In some embodiments, X3 is optionally substituted (R-βCyclopropyl) Trp. In some embodiments, X3 is optionally substituted (R-Isobutyl) D-Trp. In some embodiments, X3 is optionally substituted (R-Isobutyl) Trp. In some embodiments, X3 is optionally substituted (R-βSecbutyl) D-Trp. In some embodiments, X3 is optionally substituted (R-βSecbutyl) Trp. In some embodiments, X3 is optionally substituted (R-βNeopentyl) D-Trp. In some embodiments, X3 is optionally substituted (R-βNeopentyl) Trp. In some embodiments, X3 is optionally substituted (R-βPhenyl) D-Trp. In some embodiments, X3 is optionally substituted (R-βPhenyl) Trp. In some embodiments, X3 is optionally substituted (R-βBenzyl) D-Trp. In some embodiments, X3 is optionally substituted (R-βBenzyl) Trp. In some embodiments, X3 is optionally substituted Aza-Trp. In some embodiments, X3 is optionally substituted Aza-D-Trp. In some embodiments, X3 is optionally substituted Bzt. In some embodiments, X3 is optionally substituted D-6F-Trp. In some embodiments, X3 is optionally substituted or D-Aph(Cbm). In some embodiments, X3 is (S-βMe) D-Trp, (R-βMe) D-Trp, or D-Trp. In some embodiments, X3 is (S-βMe) D-Trp. In some embodiments, X3 is (R-βMe) D-Trp. In some embodiments, X3 is D-Trp. In some embodiments, for example in some embodiments of Formula (I′), X3 is Trp.

In some embodiments of Formula (I), (I′), (I″), (II), (III), (IV1), (IV2), (IV3), (IV4), (IV5), or (IV6), or a pharmaceutically acceptable salt thereof, X3 has a structure of:

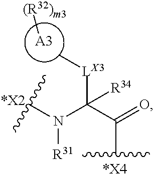

wherein:
R³¹ is hydrogen or C₁-C₈alkyl optionally substituted with one to three substituents independently selected from R^{f};
each R^{f} is independently halogen, —CN, —NO₂, —OR^{a}, —SR^{a} or —NR^{c}R^{d};
L^{X3} is a bond, —O—, —S—, —NR³³—, C₁-C₆alkylene, or C₁-C₃heteroalkylene, wherein the alkylene or heteroalkylene is optionally substituted with one or more R^{X3a}.
ring A3 is an aryl or heteroaryl;
each R³² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH) NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, aryl, heteroaryl, cycloalkyl, and heterocycloalkyl is optionally substituted with one or more R^{32a}, or
   two R³² are taken together to form ═O, ═S, or ═N(R^{a});
m3 is 0, 1, 2, 3, 4, or 5;
   each R^{32a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR³)R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R³), wherein each of the alkyl, heteroalkyl, alkenyl, alkynyl, aryl, heteroaryl, cycloalkyl, or heterocycloalkyl is optionally substituted with one or more R^{e}; or, one of R^{32a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R³³ is hydrogen or C₁-C₃alkyl;
R³⁴ is hydrogen or C₁-C₃alkyl;
each R^{X3a} is independently halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X3a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl, and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X2 represents the point of attachment to X2; and
*X4 represents the point of attachment to X4.

In some embodiments of Formula (I), (I′), (I″), (II), (III), (IV1), (IV2), (IV3), (IV4), (IV5), or (IV6), or a pharmaceutically acceptable salt thereof, ring A3 is a C₆-C₁₀aryl or a 5-10-membered heteroaryl. In some embodiments, ring A3 is a C₆-C₁₀aryl. In some embodiments, ring A3 is a 5-10-membered heteroaryl. In some embodiments, ring A3 is a phenyl, naphthyl, pyridinyl, indolyl, azaindolyl, indazolyl, benzimidazolyl, pyrimidazolyl, pyrazolo[1,5-a]pyridinyl, benzofuran, benzothiophene, quinolinyl, or isoquinolinyl. In some embodiments, ring A3 is phenyl or naphthyl. In some embodiments, ring A3 is indolyl or azaindolyl. In some embodiments, ring A3 is a phenyl. In some embodiments, ring A3 is a naphthyl. In some embodiments, ring A3 is a pyridinyl. In some embodiments, ring A3 is a benzofuran. In some embodiments, ring A3 is a benzothiophene. In some embodiments, ring A3 is an indolyl. In some embodiments, ring A3 is an azaindolyl. In some embodiments, ring A3 is an indazolyl. In some embodiments, ring A3 is a benzimidazolyl. In some embodiments, ring A3 is a pyrimidazolyl. In some embodiments, ring A3 is a pyrazolo[1,5-a]pyridinyl. In some embodiments, ring A3 is a quinolinyl. In some embodiments, ring A3 is a isoquinolinyl.

In some embodiments of Formula (I), (I′), (I″), (II), (III), (IV1), (IV2), (IV3), (IV4), (IV5), or (IV6), or a pharmaceutically acceptable salt thereof, m3 is 0 or 1. In some embodiments, m3 is 0. In some embodiments, m3 is 1.

In some embodiments of Formula (I), (I′), (I″), (II), (III), (IV1), (IV2), (IV3), (IV4), (IV5), or (IV6), or a pharmaceutically acceptable salt thereof, X3 has a structure of:

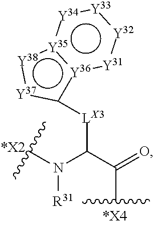

wherein:
Y³¹ is N, CH, or CR³²;
Y³² is N, CH, or CR³²;
Y³³ is N, CH, or CR³²;
Y³⁴ is N, CH, or CR³²;
Y³⁵ is N, or C;
Y³⁶ is N or C;
Y³⁷ is N, CH, or CR³²; and
Y³⁸ is O, S, N or NH.

In some embodiments, no more than two of Y³¹, Y³², Y³³, Y³⁴, Y³⁵, Y³⁶, and Y³⁷ are N. In some embodiments, Y³¹ is N. In some embodiments, Y³¹ is CH. In some embodiments, Y³² is N. In some embodiments, Y³² is CH. In some embodiments, Y³³ is N. In some embodiments, Y³³ is CH. In some embodiments, Y³⁴ is N. In some embodiments, Y³⁴ is CH. In some embodiments, Y³⁵ is N. In some embodiments, Y³⁵ is C. In some embodiments, Y³⁶ is N. In some embodiments, Y³⁶ is C. In some embodiments, Y³⁷ is N. In some embodiments, Y³⁷ is CH. In some embodiments, Y³⁸ is N. In some embodiments, Y³⁸ is NH. In some embodiments, Y³⁸ is S. In some embodiments, Y³⁸ is O.

In some embodiments of Formula (I), (I′), (I″), (II), (III), (IV1), (IV2), (IV3), (IV4), (IV5), or (IV6), or a pharmaceutically acceptable salt thereof, each R³² is independently C₁-C₆alkyl, C₁-C₆haloalkyl, C₁-C₆hydroxyalkyl, C₁-C₆aminoalkyl, C₁-C₆heteroalkyl, halogen, CN, —NO₂, —OR^{a}, —SR^{a}, —SF₅, or —NR^{c}R^{d}, wherein each of the alkyl and heteroalkyl is optionally substituted with one or more R^{32a} In some embodiments, each R³² is independently C₁-C₆alkyl, C₁-C₆haloalkyl, halogen, CN, —OR^{a}, —SR^{a}, or —NR^{c}R^{d}, wherein each of the alkyl and heteroalkyl is optionally substituted with one or more R^{32a}. In some embodiments, R³² is hydrogen or halogen. In some embodiments, R³² is hydrogen or fluoro. In some embodiments, R³² is halogen. In some embodiments, R³² is fluoro. In some embodiments, one of R³² is a CG. In some embodiments, the CG is an azide or a terminal alkyne. In some embodiments the CG is an azide. In some embodiments, the CG is a terminal alkyne.

In some embodiments of Formula (I), (I′), (I″), (II), (III), (IV1), (IV2), (IV3), (IV4), (IV5), or (IV6), or a pharmaceutically acceptable salt thereof, R³¹ is hydrogen or C₁-C₃alkyl optionally substituted with one to three substituents independently selected from R^{f}. In some embodiments, R³¹ is hydrogen. In some embodiments, R³¹ is C₁-C₃alkyl. In some embodiments, R³¹ is methyl.

In some embodiments of Formula (I), (I′), (I″), (II), (III), (IV1), (IV2), (IV3), (IV4), (IV5), or (IV6), or a pharmaceutically acceptable salt thereof, L^{X3} is C₁-C₃alkylene, optionally substituted with one to three R^{X3a} independently selected from C₁-C₃alkyl (e.g., methyl), phenyl, C₁-C₃alkylene(phenyl), C₃-C₆cycloalkyl, or C₁-C₃alkylene (C₃-C₆cycloalkyl). In some embodiments, L^{X3} is —CH₂—, —CH(CH₃)—, —C(CH₃)₂—, —CH(iPr)-, CH(benzyl)-, —CH(cyclopropyl)-, —CH(CH₂—CH(CH₃)₂)—, —CH(CH₂—C(CH₃)₃)—, or —CH(CH₂—CH₂—CH₃)—. In some embodiments, L^{X3} is —CH₂— or —CH(CH₃)—.

In some embodiments of Formula (I), (I′), (I″), (II), (III), (IV1), (IV2), (IV3), (IV4), (IV5), or (IV6), or a pharmaceutically acceptable salt thereof, X3 is:

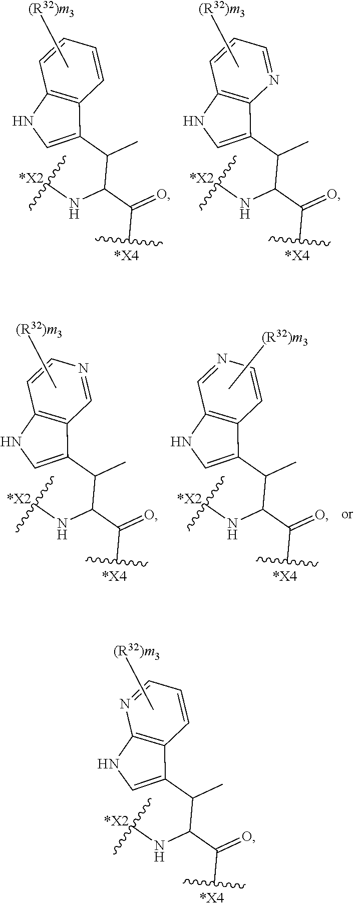

wherein
each R³² is independently C₁-C₆alkyl, C₁-C₆haloalkyl, halogen, —CN, —OR^{a}, —SR^{a}, or —NR^{c}R^{d};
m3 is 0, 1, or 2;
*X2 represents the point of attachment to X2; and
*X4 represents the point of attachment to X4.

In some embodiments, X3 is

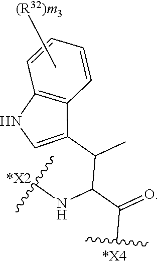

In some embodiments, X3 is

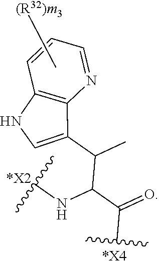

In some embodiments, X3 is

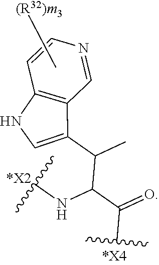

In some embodiments, X3 is

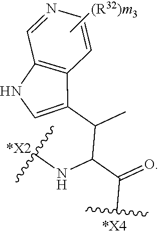

In some embodiments, X3 is

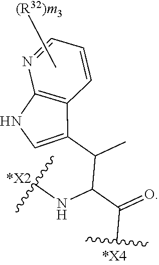

In some embodiments, each R³² is independently methyl, ethyl, isopropyl, C₁-C₂haloalkyl, halogen, —CN, —OH, —OMe, —SH, —SMe, —NH₂, —NHMe, or —N(Me)₂. In some embodiments, m3 is 0. In some embodiments, m3 is 1 or 2. In some embodiments, m3 is 1. In some embodiments, m3 is 2. In some embodiments, R³² is halogen and m3 is 1.

In some embodiments, modifications to X4 have been shown to improve conjugate biodistribution as determined by measuring conjugate concentrations in tissues by mass spectrometry. A conjugate having improved biodistribution exhibits distribution in desired tissues and decreased distribution in undesired tissues. In some embodiments, a conjugate described herein has improved biodistribution as determined by measuring tissue pharmacokinetics when X4 is a non-natural amino acid having a side chain comprising an amine. In some embodiments, the amine comprises a primary amine, a secondary amine, a tertiary amine, or a quaternary amine. In some embodiments, the amine comprises a primary amine, a secondary amine, or a tertiary amine. In some embodiments, the amine comprises a primary amine. In some embodiments, the amine comprises a secondary amine or a tertiary amine. In some embodiments, the amine comprises a secondary amine. In some embodiments, the amine comprises a tertiary amine. In some embodiments, a conjugate described herein has improved biodistribution as determined by tissue pharmacokinetics when X4 is an amino acid comprising azetidinyl, pyrrolidinyl, piperidinyl, piperazinyl, or morpholinyl.

In some embodiments of Formula (I), (I′), (II), (III), (IV1), (IV2), (IV3), (IV4), (IV5), or (IV6), or a pharmaceutically acceptable salt thereof, X4 is a non-natural amino acid having a side chain comprising an amine. In some embodiments, the amine comprises a primary amine, a secondary amine, a tertiary amine, or a quaternary amine. In some embodiments, the amine comprises a primary amine, a secondary amine, or a tertiary amine. In some embodiments, the amine comprises a primary amine. In some embodiments, the amine comprises a secondary amine or a tertiary amine. In some embodiments, the amine comprises a secondary amine. In some embodiments, the amine comprises a tertiary amine. In some embodiments, X4 is a non-natural amino acid having a side chain comprising an amine, and wherein the side chain comprises azetidinyl, pyrrolidinyl, piperidinyl, piperazinyl, or morpholinyl. In some embodiments, X4 is Cba3N, Chg4N, Cha4N, Cha4NH2, Ser(3-azetidine), PipzaA, 3-Azetidine-hAla, or Pic4, each of which is further optionally substituted. In some embodiments, X4 is a Lys derivative. In some embodiments, X4 is NMe-Lys, Lys(Me), Lys(diMe), Lys(iPr), Chg4N, Cha4N, 4-oxa-Lys, or 3-Azetidine-hAla. In some embodiments, X4 is Cba3N, Chg4N, Cha4N, Cha4NH2, Ser(3-azetidine), PipzaA, 3-Azetidine-hAla, Pic4, NMe-Lys, Lys(Me), Lys(diMe), Lys(iPr), or 4-oxa-Lys. In some embodiments, X4 is (D/L) Hly. In some embodiments, X4 is optionally substituted Chg4N. In some embodiments, X4 is optionally substituted Cha4N. In some embodiments, X4 is optionally substituted Ser(3-azetidine). In some embodiments, X4 is optionally substituted PipzaA. In some embodiments, X4 is optionally substituted 3-Azetidine-hAla. In some embodiments, X4 is optionally substituted NMe-Lys. In some embodiments, X4 is optionally substituted Lys(Me). In some embodiments, X4 is optionally substituted Lys(iPr). In some embodiments, X4 is optionally substituted 4-oxa-Lys. In some embodiments, X4 is Chg4N. In some embodiments, X4 is Cha4N. In some embodiments, X4 is Ser(3-azetidine). In some embodiments, X4 is PipzaA. In some embodiments, X4 is 3-Azetidine-hAla. In some embodiments, X4 is Pic4. In some embodiments, X4 NMe-Lys. In some embodiments, X4 is Lys(Me). In some embodiments, X4 is Lys(iPr). In some embodiments, X4 is 4-oxa-Lys. In some embodiments, X4 is D-Lys. In some embodiments, X4 is azaLys. In some embodiments, X4 is Lys(triMe). In some embodiments, X4 is Nva(NH—NH2).

In some embodiments of Formula (I), (I′), (II), (III), (IV1), (IV2), (IV3), (IV4), (IV5), or (IV6), or a pharmaceutically acceptable salt thereof, X4 has a structure of:

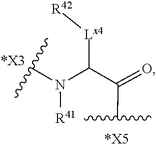

wherein,
R⁴¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
L^{X4} is a bond, —O—, —S—, —NR⁴³—, C₁-C₆alkylene, C₁-C₆heteroalkylene, C₃-C₆cycloalkyl, or 3- to 6-membered heterocycloalkyl, wherein the alkylene, heteroalkylene, cycloalkyl, or heterocycloalkyl is optionally substituted with one or more R^{X4a},
R⁴² is —NR⁴⁴R⁴⁵ or a heterocycloalkyl comprising one or more ring nitrogen atoms, wherein the heterocycloalkyl is optionally substituted with one or more R^{42a};
each R^{42a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, ═O, ═S, or ═N(R^{a}), wherein each of the alkyl, heteroalkyl, alkenyl, and alkynyl is optionally substituted with one or more R^{e}; or, one of R^{42a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R⁴³ is hydrogen or C₁-C₃alkyl;
R⁴⁴ and R⁴⁵ are each independently hydrogen, C₁-C₃alkyl, aryl, heteroaryl, —C₁-C₃alkylene-aryl, or —C₁-C₆alkylene-heteroaryl; or R⁴⁴ and R⁴⁵ are taken together to form a 3- to 6-membered heterocycloalkyl, wherein each of the alkyl, aryl, heteroaryl, and heterocycloalkyl are optionally substituted with one or more R^{42a};
each R^{X4a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, ═O, ═S, or ═N(R³), wherein each of the alkyl, heteroalkyl, alkenyl, and alkynyl is optionally substituted with one or more R^{e}; or
or two R^{X4a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O) OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X3 represents the point of attachment to X3; and
*X5 represents the point of attachment to X5.

In some embodiments of Formula (I), (I′), (II), (III), (IV1), (IV2), (IV3), (IV4), (IV5), or (IV6), or a pharmaceutically acceptable salt thereof, R⁴² is a 4- to 6-membered N-containing heterocycloalkyl, which is optionally substituted with one or more R^{42a}. In some embodiments, R⁴² is azetidinyl, pyrrolidinyl, piperidinyl, piperazinyl, or morpholinyl, which is optionally substituted with one to four R^{42a} In some embodiments, R⁴² is azetidinyl, piperidinyl, or piperazinyl, each of which is optionally substituted with one to four R^{42a}. In some embodiments, R⁴² is azetidinyl optionally substituted with one to four R^{42a}. In some embodiments, R⁴² is pyrrolidinyl optionally substituted with one to four R^{42a}. In some embodiments, R⁴² is piperidinyl optionally substituted with one to four R^{42a}. In some embodiments, R⁴² is piperazinyl optionally substituted with one to four R^{42a}. In some embodiments, R⁴² is morpholinyl optionally substituted with one to four R^{42a}. In some embodiments, R⁴² is

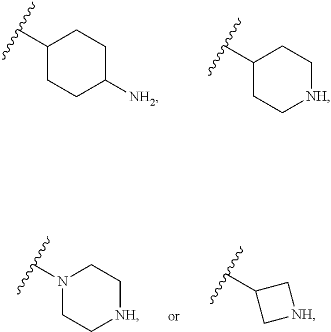 [SMILES: *C1CCC(N)CC1 ; *C1CCNCC1 ; *N1CCNCC1 ; *C1CNC1]

each of which is optionally substituted with 1 or 2 substituents selected from R^{42a}. In some embodiments, R⁴² is

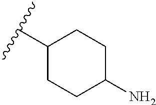 [SMILES: *C1CCC(N)CC1]

optionally substituted with 1 or 2 substituents selected from R^{42a}. In some embodiments, R⁴² is,

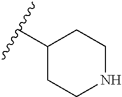 [SMILES: *C1CCNCC1]

optionally substituted with 1 or 2 substituents selected from R^{42a}. In some embodiments, R⁴² is

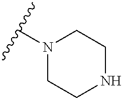 [SMILES: *N1CCNCC1]

optionally substituted with 1 or 2 substituents selected from R^{42a}. In some embodiments, R⁴² is

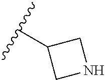 [SMILES: *C1CNC1]

optionally substituted with 1 or 2 substituents selected from R^{42a}. In some embodiments, each R^{42a} is independently halogen, C₁-C₆alkyl, C₁-C₆haloalkyl, —OR^{a}, or ═O. In some embodiments, each R^{42a} is C₁-C₃alkyl, C₁-C₃haloalkyl. —OH, or —O. In some embodiments, R^{42a} is C₁-C₃alkyl. In some embodiments, R⁴² is —NR⁴⁴R⁴⁵.

In some embodiments, R⁴⁴ and R⁴⁵ are each independently hydrogen, C₁-C₃alkyl, aryl, heteroaryl, —C₁-C₆alkylene-aryl, or —C₁-C₆alkylene-heteroaryl, wherein each of the alkyl, aryl, and heteroaryl are optionally substituted with one or more R^{42a}. In some embodiments, R⁴⁴ and R⁴⁵ are each independently hydrogen, methyl, ethyl, isopropyl, phenyl, —C₁-C₃alkylene-phenyl, or —NH₂, wherein each of the methyl, ethyl, isopropyl, phenyl, and alkyl are each optionally substituted with 1 or 2 substituents selected from R^{42a}. In some embodiments, R⁴⁴ and R⁴⁵ are each independently hydrogen, methyl, ethyl, isopropyl, phenyl, —C₁-C₃alkylene-phenyl, or —NH₂. In some embodiments, R⁴⁴ and R⁴⁵ are each independently hydrogen, methyl, ethyl, or isopropyl. In some embodiments, R⁴⁴ and R⁴⁵ are each independently hydrogen or methyl. In some embodiments, R⁴⁴ and R⁴⁵ are each independently hydrogen or ethyl. In some embodiments, R⁴⁴ and R⁴⁵ are each independently hydrogen or isopropyl. In some embodiments, R⁴⁴ and R⁴⁵ are each independently hydrogen or phenyl. In some embodiments, R⁴⁴ and R⁴⁵ are each independently hydrogen or —C₁-C₃alkylene-phenyl. In some embodiments, R⁴⁴ and R⁴⁵ are each independently hydrogen or —NH₂. In some embodiments, one of R^{42a} is a CG. In some embodiments, R^{42a} is a CG. In some embodiments, the CG is an azide or a terminal alkyne.

In some embodiments of Formula (I), (I′), (II), (III), (IV1), (IV2), (IV3), (IV4), (IV5), or (IV6), or a pharmaceutically acceptable salt thereof, L^{X4} is a bond. In some embodiments, L^{X4} is a C₁-C₄alkylene (e.g., —CH₂—), wherein the alkylene is optionally substituted with 1 or 2 substituents independently selected from R^{X4a}. In some embodiments, L^{X4} is a C₁-C₄heteroalkylene (e.g., —CH₂OCH₂CH₂—) wherein the heteroalkylene is optionally substituted with 1 or 2 substituents independently selected from R^{X4a}. In some embodiments, R^{X4a} is —F, —Me, or —OH.

In some embodiments of Formula (I), (I′), (II), (III), (IV1), (IV2), (IV3), (IV4), (IV5), or (IV6), or a pharmaceutically acceptable salt thereof, R⁴¹ is hydrogen or C₁-C₃alkyl optionally substituted with one to three substituents independently selected from R^{f}. In some embodiments, R⁴¹ is hydrogen. In some embodiments, R⁴¹ is C₁-C₃alkyl. In some embodiments, R⁴¹ is methyl.

In some embodiments of Formula (I), (I′), (II), (III), (IV1), (IV2), (IV3), (IV4), (IV5), or (IV6), or a pharmaceutically acceptable salt thereof, X4 is

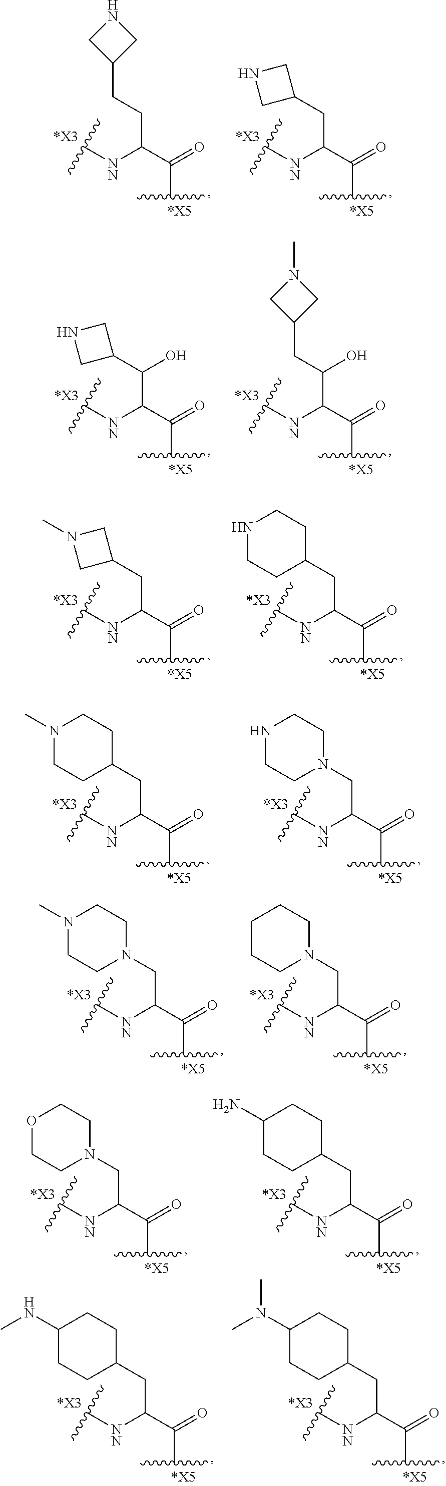

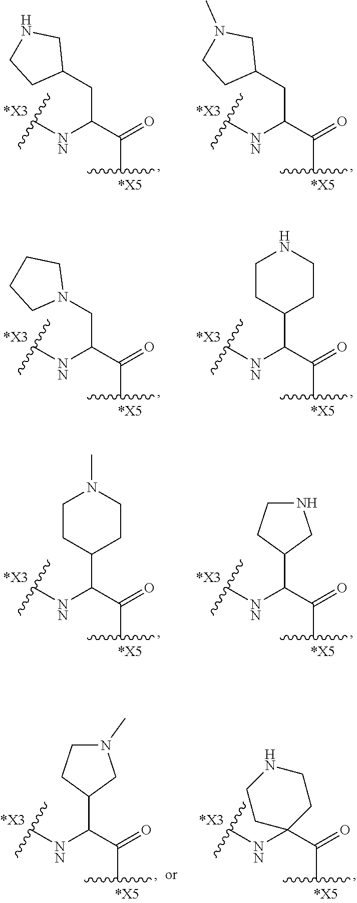

wherein
*X3 represents the point of attachment to X3; and
*X5 represents the point of attachment to X5.

In some embodiments, X4 is

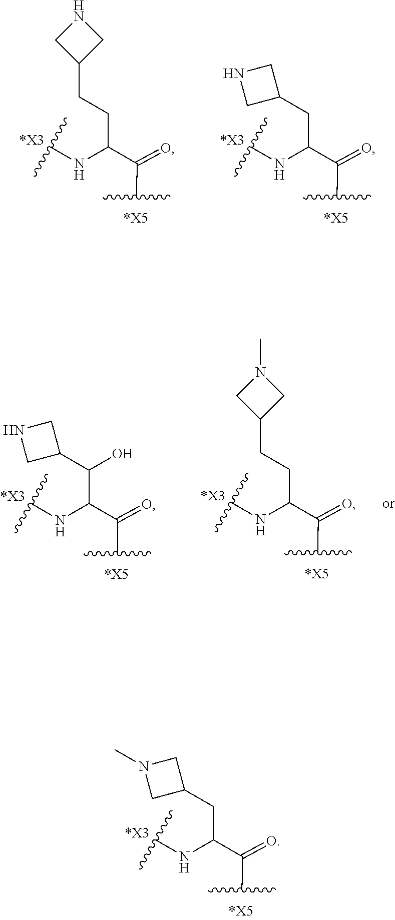 [SMILES: *NC(CCC1CNC1)C(*)=O ; *NC(CC1CNC1)C(*)=O ; *NC(C(*)=O)C(O)C1CNC1 ; *NC(CCC1CN(C)C1)C(*)=O ; *NC(CC1CN(C)C1)C(*)=O]

In some embodiments, X4 is

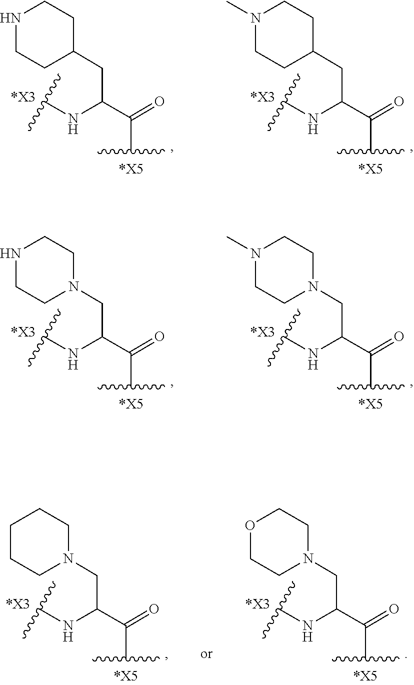 [SMILES: *NC(CC1CCNCC1)C(*)=O ; *NC(CC1CCN(C)CC1)C(*)=O ; *NC(CN1CCNCC1)C(*)=O ; *NC(CN1CCN(C)CC1)C(*)=O ; *NC(CN1CCCCC1)C(*)=O ; *NC(CN1CCOCC1)C(*)=O]

In some embodiments, X4 is

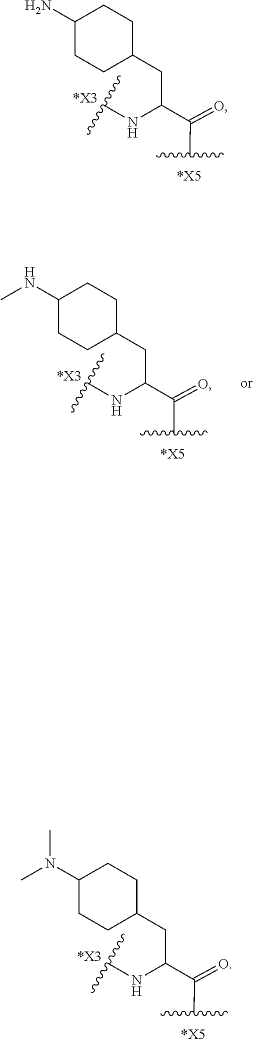 [SMILES: *NC(CC1CCC(N)CC1)C(*)=O ; *NC(CC1CCC(NC)CC1)C(*)=O ; *NC(CC1CCC(N(C)C)CC1)C(*)=O]

In some embodiments, X4 is

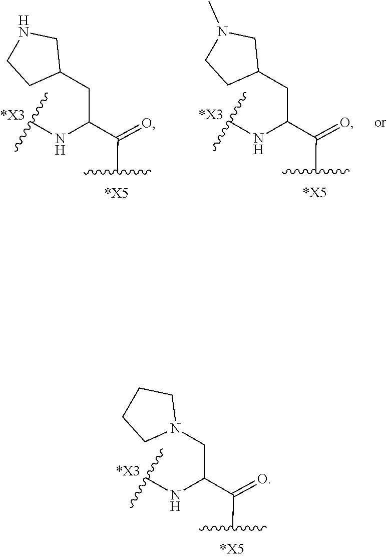 [SMILES: *NC(CC1CCNC1)C(*)=O ; *NC(CC1CCN(C)C1)C(*)=O ; *NC(CN1CCCC1)C(*)=O]

In some embodiments, X4 is

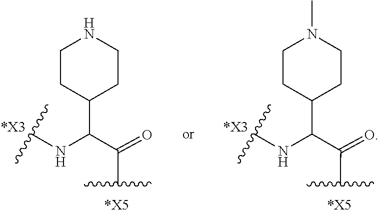 [SMILES: *NC(C(*)=O)C1CCNCC1 ; *NC(C(*)=O)C1CCN(C)CC1]

In some embodiments, X4 is

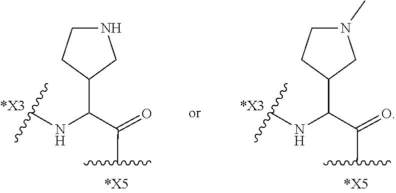 [SMILES: *NC(C(*)=O)C1CCNC1 ; *NC(C(*)=O)C1CCN(C)C1]

In some embodiments, X4 is.

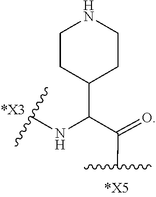 [SMILES: *NC(C(*)=O)C1CCNCC1]

In some embodiments of Formula (I), (I′), (II), (III), (IV1), (IV2), (IV3), (IV4), (IV5), or (IV6), or a pharmaceutically acceptable salt thereof, X4 is

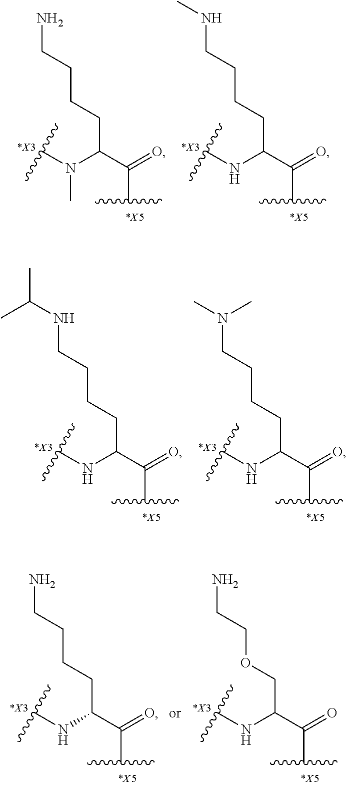 [SMILES: *C(=O)C(CCCCN)N(*)C ; *NC(CCCCNC)C(*)=O ; *NC(CCCCNC(C)C)C(*)=O ; *NC(CCCCN(C)C)C(*)=O ; *N[C@H](CCCCN)C(*)=O ; *NC(COCCN)C(*)=O]

wherein
*X3 represents the point of attachment to X3; and
*X5 represents the point of attachment to X5.

In some embodiments, X4 is

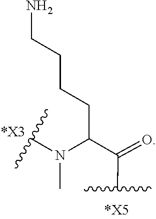 [SMILES: *C(=O)C(CCCCN)N(*)C]

In some embodiments, X4 is

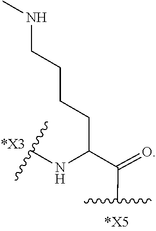 [SMILES: *NC(CCCCNC)C(*)=O]

In some embodiments, X4 is

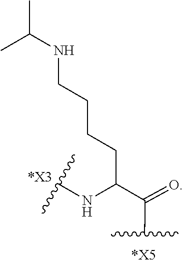 [SMILES: *NC(CCCCNC(C)C)C(*)=O]

In some embodiments, X4 is

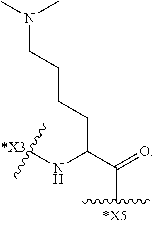 [SMILES: *NC(CCCCN(C)C)C(*)=O]

In some embodiments, X4 is

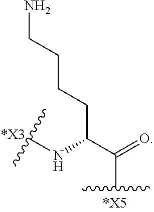 [SMILES: *N[C@H](CCCCN)C(*)=O]

In some embodiments, X4 is

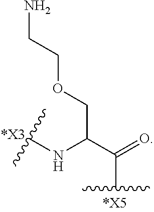 [SMILES: *NC(COCCN)C(*)=O]

In some embodiments of Formula (I), (I′), (I″), (II), (III), (IV1), (IV2), (IV3), (IV4), (IV5), or (IV6), or a pharmaceutically acceptable salt thereof, X5 a D-amino acid. In some embodiments, X5 is an L-amino acid. In some embodiments, X5 is an aliphatic amino acid or a polar amino acid. In some embodiments, X5 is Thr, Val, Ala, Ser, Pro, or a derivative thereof. In some embodiments, X5 is Thr or a derivative thereof. In some embodiments, X5 is Val or a derivative thereof. In some embodiments, X5 is Ala or a derivative thereof. In some embodiments, X5 is Ser or a derivative thereof. In some embodiments, X5 is Pro or a derivative thereof. In some embodiments, X5 is optionally substituted Thr. In some embodiments, X5 is optionally substituted Val. In some embodiments, X5 is optionally substituted Ala. In some embodiments, X5 is optionally substituted Pro. In some embodiments, X5 is optionally substituted Alt. In some embodiments, X5 is optionally substituted Cbg. In some embodiments, X5 is optionally substituted Cpg. In some embodiments, X5 is optionally substituted Cba. In some embodiments, X5 is optionally substituted Tme In some embodiments, X5 is Thr, Val, Ala, Pro, Alt, Cbg, Cpg, Cba, or Tme. In some embodiments, X5 is Thr. In some embodiments, X5 is Val. In some embodiments, X5 is Ala. In some embodiments, X5 is Pro. In some embodiments, X5 is Alt, Cbg. In some embodiments, X5 is Cpg. In some embodiments, X5 is Cba. In some embodiments, X5 is Tme.

In some embodiments of Formula (I), (I′), (I″), (II), (III), (IV1), (IV2), (IV3), (IV4), (IV5), or (IV6), or a pharmaceutically acceptable salt thereof, X5 has a structure of:

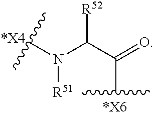

wherein,
R⁵¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
R⁵² is C₁-C₆alkyl, C₁-C₆haloalkyl, C₁-C₆hydroxyalkyl, C₁-C₆aminoalkyl, C₁-C₆heteroalkyl, C₂₋₆alkenyl, C₂₋₆alkynyl, C₃-C₆cycloalkyl, or 4- to 6-membered heterocycloalkyl, wherein each of the alkyl, heteroalkyl, aryl, heteroaryl, cycloalkyl, and heterocycloalkyl is optionally substituted with one or more R^{52a}; or
R⁵¹ and R⁵² are taken together with the intervening atoms to form a 5- to 6-membered heterocycloalkyl, which is optionally substituted with one or more R^{52a}.
each R^{52a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —SeR^{a}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR², —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR)(OR^{a}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{52a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X4 represents the point of attachment to X4; and
*X6 represents the point of attachment to X6.

In some embodiments of Formula (I), (I′), (I″), (II), (III), (IV1), (IV2), (IV3), (IV4), (IV5), or (IV6), or a pharmaceutically acceptable salt thereof, R⁵² is C₁-C₆alkyl, C₁-C₆haloalkyl, C₁-C₆hydroxyalkyl, C₁-C₆aminoalkyl, C₁-C₆heteroalkyl, C₃-C₆cycloalkyl, or 4- to 6-membered heterocycloalkyl, wherein each of the alkyl, heteroalkyl, cycloalkyl, and heterocycloalkyl is optionally substituted with one or more R^{52a}. In some embodiments, R⁵² is C₁-C₆alkyl, C₁-C₆heteroalkyl, or C₃-C₆cycloalkyl, wherein each of the alkyl, heteroalkyl, and cycloalkyl is optionally substituted with one or more R^{52a}. In some embodiments, R⁵² is C₁-C₆alkyl, C₁-C₆heteroalkyl, or C₃-C₆cycloalkyl, wherein each of the alkyl, heteroalkyl, and cycloalkyl is optionally substituted with one to three substituents independently selected from —OH, —OMe, and C₃-C₄cycloalkyl. In some embodiments, R⁵² is C₃-C₆cycloalkyl or C₁-C₃alkyl optionally substituted with one to three substituents independently selected from —OH, —OMe, and C₃-C₄cycloalkyl. In some embodiments, R⁵² is C₁-C₃alkyl optionally substituted with R^{52a}. In some embodiments, R⁵² is C₁-C₃alkyl optionally substituted with —OH, —OMe, or C₃-C₆cycloalkyl. In some embodiments, R⁵² is C₃-C₆cycloalkyl optionally substituted with —OH or —OMe. In some embodiments, R⁵² is C₃-C₆cycloalkyl.

In some embodiments of Formula (I), (I′), (I″), (II), (III), (IV1), (IV2), (IV3), (IV4), (IV5), or (IV6), or a pharmaceutically acceptable salt thereof, one of R^{52a} is a CG. In some embodiments, R^{52a} is a CG. In some embodiments, the CG is an azide or a terminal alkyne.

In some embodiments of Formula (I), (I′), (I″), (II), (III), (IV1), (IV2), (IV3), (IV4), (IV5), or (IV6), or a pharmaceutically acceptable salt thereof, R⁵¹ is hydrogen or C₁-C₃alkyl optionally substituted with one to three substituents independently selected from R^{f}. In some embodiments, R⁵¹ is hydrogen. In some embodiments, R⁵¹ is C₁-C₃alkyl. In some embodiments, R⁵¹ is methyl. In some embodiments, R⁵¹ and R⁵² are taken together and the intervening atoms to form a 5- to 6-membered heterocycloalkyl, which is optionally substituted with one or more R^{52a}. In some embodiments, R⁵¹ and R⁵² are taken together and the intervening atoms to form a 5-membered heterocycloalkyl, which is optionally substituted with one or more R^{52a}. In some embodiments, R⁵¹ and R⁵² are taken together and the intervening atoms to form a 6-membered heterocycloalkyl, which is optionally substituted with one or more R^{52a} In some embodiments, R⁵¹ and R⁵² are taken together and the intervening atoms to form a 5-membered heterocycloalkyl, which is optionally substituted with one or two substitutions selected from C₁-C₃alkyl, C₁-C₃haloalkyl, C₁-C₃hydroxyalkyl, —OH, and —OMe. In some embodiments, R⁵¹ and R⁵² are taken together and the intervening atoms to form a 5-membered heterocycloalkyl.

In some embodiments of Formula (I), (I′), (I″), (II), (III), (IV1), (IV2), (IV3), (IV4), (IV5), or (IV6), or a pharmaceutically acceptable salt thereof, X6 is a D-amino acid. In some embodiments, X6 is an L-amino acid. In some embodiments, X6 is an aromatic amino acid, a hydrophilic amino acid, or Gly, or a derivative thereof. In some embodiments, X6 is an aromatic amino acid or a derivative thereof. In some embodiments, X6 is Phe, His, Tyr, or a derivative thereof. In some embodiments, X6 is Phe or a derivative thereof. In some embodiments, X6 is His or a derivative thereof. In some embodiments, X6 is Tyr or a derivative thereof. In some embodiments, X6 is a hydrophilic amino acid or a derivative thereof. In some embodiments, X6 is Ser, Asn, or a derivative thereof. In some embodiments, X6 is Ser or a derivative thereof. In some embodiments, X6 is Asn or a derivative thereof. In some embodiments, X6 is Gly or a derivative thereof. In some embodiments, X6 is Ala or a derivative thereof. In some embodiments, X6 is Val or a derivative thereof. In some embodiments, X6 is Pro or a derivative thereof. In some embodiments, X6 is Phe, Ala, Gly, Ser, His, Tyr, Asn, Pro, or a derivative thereof. In some embodiments, X6 is Phe, NMe-Phe, (S-βMe) Phe, (R-βMe) Phe, His, Mpd, Ala, D-Ala, Gly, Pro, Ser, Ser(Ph), 3Pal, 4Pal, Cha, 3-(Aminocarbonyl)-Phe, F4COO, Phg, G(cPr), Asn, Tyr, meta-Tyr, or 3N-Tyr, each of which is optionally further substituted. In some embodiments, X6 is optionally substituted Phe. In some embodiments, X6 is optionally substituted NMe-Phe. In some embodiments, X6 is optionally substituted (S-βMe) Phe. In some embodiments, X6 is optionally substituted (R-βMe) Phe. In some embodiments, X6 is optionally substituted His. In some embodiments, X6 is optionally substituted Mpd. In some embodiments, X6 is optionally substituted Ala. In some embodiments, X6 is optionally substituted Val. In some embodiments, X6 is optionally substituted D-Ala. In some embodiments, X6 is optionally substituted Gly. In some embodiments, X6 is optionally substituted Pro. In some embodiments, X6 is optionally substituted Ser. In some embodiments, X6 is optionally substituted Ser(Ph). In some embodiments, X6 is optionally substituted 3Pal. In some embodiments, X6 is optionally substituted 4Pal. In some embodiments, X6 is optionally substituted Cha. In some embodiments, X6 is optionally substituted 3-(Aminocarbonyl)-Phe. In some embodiments, X6 is optionally substituted F4COO. In some embodiments, X6 is optionally substituted Phg. In some embodiments, X6 is optionally substituted G(cPr). In some embodiments, X6 is optionally substituted Asn. In some embodiments, X6 is optionally substituted Tyr. In some embodiments, X6 is optionally substituted meta-Tyr. In some embodiments, X6 is optionally substituted 3N-Tyr. In some embodiments, X6 is Phe. In some embodiments, X6 is NMe-Phe. In some embodiments, X6 is (S-βMe) Phe. In some embodiments, X6 is (R-βMe) Phe. In some embodiments, X6 is His. In some embodiments, X6 is Mpd. In some embodiments, X6 is Ala. In some embodiments, X6 is D-Ala. In some embodiments, X6 is Val. In some embodiments, X6 Gly. In some embodiments, X6 is Pro. In some embodiments, X6 is Ser. In some embodiments, X6 is Ser(Ph). In some embodiments, X6 is 3Pal. In some embodiments, X6 is 4Pal. In some embodiments, X6 is Cha. In some embodiments, X6 is 3-(Aminocarbonyl)-Phe. In some embodiments, X6 is Phg. In some embodiments, X6 is G(cPr). In some embodiments, X6 is Asn. In some embodiments, X6 is Tyr. In some embodiments, X6 is meta-Tyr. In some embodiments, X6 is 3N-Tyr.

In some embodiments of Formula (I), (I′), or (I″), or a pharmaceutically acceptable salt thereof, X6 has a structure of:

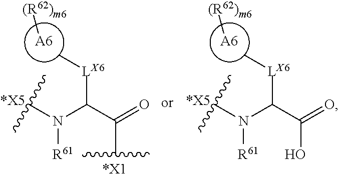

wherein:
R⁶¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
ring A6 is a cycloalkyl, heterocycloalkyl, aryl, or heteroaryl;
each R⁶² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH) NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, aryl, heteroaryl, cycloalkyl, and heterocycloalkyl is optionally substituted with one or more R^{62a}; or
   two R⁶² are taken together to form ═O, ═S, or ═N(R^{a});
L^{X6} is a bond, —O—, —S—, —NR⁶³—, C₁-C₆alkylene, or C₁-C₃heteroalkylene, wherein the alkylene or heteroalkylene is optionally substituted with one or more R^{X6a}.
m6 is 0, 1, 2, 3, 4, or 5;
each R^{62a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{62a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R⁶³ is hydrogen or C₁-C₃ alkyl;
R^{X6a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X6a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene (cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X5 represents the point of attachment to X5; and
*X1 represents the point of attachment to X1.

In some embodiments of Formula (I), (I′), (I″), (II), (III), (IV1), (IV2), (IV3), (IV4), (IV5), or (IV6), or a pharmaceutically acceptable salt thereof, X6 has a structure of:

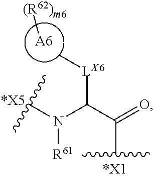

wherein:
R⁶¹ is hydrogen or C₁-C₆alkyl optionally substituted with one to three substituents independently selected from R^{f};
each R^{f} is independently halogen, —CN, —NO₂, —OR^{a}, —SR^{a} or —NR^{c}R^{d};
ring A6 is a cycloalkyl, heterocycloalkyl, aryl, or heteroaryl;
each R⁶² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH) NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, or heterocycloalkyl, wherein each of the alkyl, heteroalkyl, aryl, heteroaryl, cycloalkyl, and heterocycloalkyl is optionally substituted with one or more R^{62a}; or
   two R⁶² are taken together to form ═O, ═S, or ═N(R^{a});
L^{X6} is a bond, —O—, —S—, —NR⁶³—, C₁-C₆alkylene, or C₁-C₃heteroalkylene, wherein the alkylene or heteroalkylene is optionally substituted with one or more R^{X6a},
m6 is 0, 1, 2, 3, 4, or 5;
each R^{62a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{62a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
R⁶³ is hydrogen or C₁-C₃ alkyl;
R^{X6a} is halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X6a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C; —C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{e} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X5 represents the point of attachment to X5; and
*X1 represents the point of attachment to X1.

In some embodiments of Formula (I), (I′), (I″), (II), (III), (IV1), (IV2), (IV3), (IV4), (IV5), or (IV6), or a pharmaceutically acceptable salt thereof, ring A6 is a C₆-C₁₀aryl or a 5- to 10-membered heteroaryl. In some embodiments, ring A6 is a C₆-C₁₀aryl. In some embodiments, ring A6 is a 5- to 10-membered heteroaryl. In some embodiments, ring A6 is a phenyl or a 5- to 6-membered heteroaryl. In some embodiments, ring A6 is a phenyl. In some embodiments, ring A6 is a 5- to 6-membered heteroaryl. In some embodiments, ring A6 is phenyl, pyridinyl, pyrimidinyl, or imidazolyl. In some embodiments, ring A6 is pyridinyl, pyrimidinyl, or imidazolyl. In some embodiments, ring A6 is pyridinyl. In some embodiments, ring A6 is, pyrimidinyl. In some embodiments, ring A6 is imidazolyl. In some embodiments, ring A6 is C₅-C₇cycloalky or 5- to 7-membered heterocycloalky. In some embodiments, ring A6 is C₅-C₇cycloalky. In some embodiments, ring A6 is cyclohexyl. In some embodiments, ring A6 is 5- to 7-membered heterocycloalky. In some embodiments, ring A6 is piperidinyl, piperazinyl, morpholino, or tetrahydropyranyl.

In some embodiments of Formula (I), (I′), (I″), (II), (III), (IV1), (IV2), (IV3), (IV4), (IV5), or (IV6), or a pharmaceutically acceptable salt thereof, each R⁶² is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, halogen, CN, —NO₂, —OR^{a}, —SR^{a}, —NR^{c}R^{d}, —NR^{a}C(═NH) NR^{c}R^{d}, —NR^{a}S(═O)₂NR^{c}R^{d}, —C(═O)R^{a}, —C(═O)OR^{a}, —OC(═O)R^{a}, —NR^{a}C(═O)R^{a}, —NR^{a}C(═O)OR^{a}, —C(═O)NR^{c}R^{d}, C₆-C₁₀aryl, 5- to 10-membered heteroaryl, C₃-C₆cycloalkyl, or 5- to 6-membered heterocycloalkyl, wherein each of the alkyl, heteroalkyl, aryl, heteroaryl, cycloalkyl, and heterocycloalkyl is optionally substituted with one or more R^{62a}. In some embodiments, each R⁶² is independently C₁-C₃alkyl, —OR^{a}, —SR^{a}, —NR^{c}R^{d}, halogen-C(═O)R^{a}, —C(═O)OR^{a}, or —C(═O)NR^{c}R^{d}. In some embodiments, one of R^{62a} is a CG. In some embodiments, the CG is an azide or a terminal alkyne. In some embodiments, the CG is an azide. In some embodiments, the CG is a terminal alkyne.

In some embodiments of Formula (I), (I′), (I″), (II), (III), (IV1), (IV2), (IV3), (IV4), (IV5), or (IV6), or a pharmaceutically acceptable salt thereof, R⁶¹ is hydrogen or C₁-C₃alkyl optionally substituted with one to three substituents independently selected from R^{f}. In some embodiments, R⁶¹ is hydrogen. In some embodiments, R⁶¹ is C₁-C₃alkyl. In some embodiments, R⁶¹ is methyl.

In some embodiments of Formula (I), (I′), (I″), (II), (III), (IV1), (IV2), (IV3), (IV4), (IV5), or (IV6), or a pharmaceutically acceptable salt thereof, L^{X6} is bond, C₁-C₆alkylene, or C₁-C₇heteroalkylene, wherein the alkylene and heteroalkylene are independently optionally substituted with one to three substituents selected from R^{X6a}. In some embodiments, L^{X6} is bond, C₁-C₃alkylene, or C₁-C₃heteroalkylene, wherein the alkylene and heteroalkylene are independently optionally substituted with one to three C₁-C₃alkyl (e.g., methyl). In some embodiments, L^{X6} is bond. In some embodiments, L^{X6} is C₁-C₃alkylene. In some embodiments, L^{X6} is —CH₂—, —CH(CH₃)—, or —C(CH₃)₂—. In some embodiments, L^{X6} is C₁-C₃heteroalkylene. In some embodiments, L^{X6} is —CH₂NH—, —CH₂N(Me)-, or —CH₂O—.

In some embodiments of Formula (I), (I′), (I″), (II), (III), (IV1), (IV2), (IV3), (IV4), (IV5), or (IV6), or a pharmaceutically acceptable salt thereof, m6 is 0. In some embodiments, m6 is 1, 2, 3, 4, or 5. In some embodiments, m6 is 1 or 2. In some embodiments, m6 is 0 or 1. In some embodiments, m6 is 1.

In some embodiments of Formula (I), (I′), or (I″), or a pharmaceutically acceptable salt thereof, X6 has a structure of:

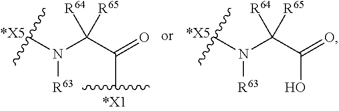

wherein:
R⁶³ is hydrogen or C₁-C₆alkyl optional substituted with one to three substituents independently selected from R^{f};
each R^{f} is independently halogen, —CN, —NO₂, —OR^{a}, —SR^{a} or —NR^{c}R^{d};
R⁶⁴ is hydrogen, C₁-C₆alkyl, C₁-C₆haloalkyl, C₁-C₆hydroxyalkyl, C₁-C₆aminoalkyl, C₁-C₆heteroalkyl, C₃-C₆cycloyalkyl, or 3- to 6-membered heterocycloalkyl, wherein each of the alkyl, heteroalkyl, cycloalkyl, and heterocycloalkyl is optionally substituted with one or more R^{64a}.
R⁶⁵ is hydrogen, halogen, or C₁-C₆alkyl, wherein the alkyl is optionally substituted with one or more R^{64a}; or
R⁶⁴ and R⁶⁵ are taken together with the carbon to which they are attached to form a C₃-C₆cycloyalkyl or a 4- to 6-membered heterocycloalkyl, each of which is optionally substituted with one or more R^{64a}; or
R⁶³ and R⁶⁴ are taken together with the intervening atoms to form a 5- to 6-membered heterocycloalkyl, which is optionally substituted with one or more R^{64a};
each R^{64a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R³, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{64a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X5 represents the point of attachment to X5; and
*X1 represents the point of attachment to X1.

In some embodiments of Formula (I), (I′), (I″), (II), (III), (IV1), (IV2), (IV3), (IV4), (IV5), or (IV6), or a pharmaceutically acceptable salt thereof, X6 has a structure of:

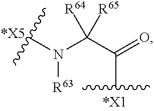

wherein:
R⁶³ is hydrogen or C₁-C₆alkyl optional substituted with one to three substituents independently selected from R^{f};
each R^{f} is independently halogen, —CN, —NO₂, —OR^{a}, —SR^{a} or —NR^{c}R^{d};
R⁶⁴ is hydrogen, C₁-C₆alkyl, C₁-C₆haloalkyl, C₁-C₆hydroxyalkyl, C₁-C₆aminoalkyl, C₁-C₆heteroalkyl, C₃-C₆cycloyalkyl, or 3- to 6-membered heterocycloalkyl, wherein each of the alkyl, heteroalkyl, cycloalkyl, and heterocycloalkyl is optionally substituted with one or more R^{64a};
R⁶⁵ is hydrogen, halogen, or C₁-C₆alkyl, wherein the alkyl is optionally substituted with one or more R^{64a}, or
R⁶⁴ and R⁶⁵ are taken together with the carbon to which they are attached to form a C₃-C₆cycloyalkyl or a 4- to 6-membered heterocycloalkyl, each of which is optionally substituted with one or more R^{64a}; or
R⁶³ and R⁶⁴ are taken together with the intervening atoms to form a 5- to 6-membered heterocycloalkyl, which is optionally substituted with one or more R^{64a};
each R^{64a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, alkynyl, aryl, heteroaryl, cycloalkyl, or heterocycloalkyl is optionally substituted with one or more R^{e}; or, one of R^{64a} is a conjugation group (CG);
CG is an optionally substituted conjugated diene, an optionally substituted tetrazine, an optionally substituted alkyne, an azide, an optionally substituted dibenzocyclooctyne (DBCO), an optionally substituted trans-cyclooctene (TCO), an optionally substituted bicyclo[6.1.0]nonyne (BCN), an optionally substituted aldehyde, an optionally substituted ketone, or an optionally substituted hydrazine;
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X5 represents the point of attachment to X5; and
*X1 represents the point of attachment to X1.

In some embodiments of Formula (I), (I′), (I″), (II), (III), (IV1), (IV2), (IV3), (IV4), (IV5), or (IV6), or a pharmaceutically acceptable salt thereof, R⁶⁴ is hydrogen, C₁-C₆alkyl, C₁-C₆hydroxyalkyl, C₁-C₆aminoalkyl, C₁-C₆heteroalkyl, or C₃-C₆cycloyalkyl, wherein each of the alkyl, heteroalkyl, and cycloalkyl is optionally substituted with one or more R^{64a}. In some embodiments, R⁶⁴ is hydrogen, C₁-C₆alkyl, C₁-C₆hydroxyalkyl, or C₁-C₆aminoalkyl, wherein the alkyl is optionally substituted with one to three R^{64a}. In some embodiments, R⁶⁴ is hydrogen or C₁-C₆alkyl, wherein the alkyl is optionally substituted with one to three R^{64a}. In some embodiments, R⁶⁴ is hydrogen or C₁-C₆alkyl optionally substituted with one to three substituents independently selected from —OH, —OMe, —C(═O)NH₂ and —C(═O)OH. In some embodiments, R⁶⁴ is hydrogen. In some embodiments, R⁶⁴ is C₁-C₆alkyl. In some embodiments, R⁶⁴ is C₁-C₃alkyl optionally substituted with one to three substituents independently selected from —OH, —OMe, —C(═O)NH₂ and —C(═O)OH. In some embodiments, R⁶⁴ is C₁-C₃alkyl optionally substituted with —OH or —OMe. In some embodiments, R⁶⁴ is methyl. In some embodiments, R⁶⁴ is C₁-C₆aminoalkyl. In some embodiments, R⁶⁴ and R⁶⁵ are taken together with the carbon to which they are attached to form a C₃-C₆cycloyalkyl, which is optionally substituted with one or more R^{64a}. In some embodiments, R⁶⁴ and R⁶⁵ are taken together with the carbon to which they are attached to form a cyclopropyl. In some embodiments, R⁶⁴ and R⁶⁵ are taken together with the carbon to which they are attached to form a cyclobutyl. In some embodiments, R⁶⁴ and R⁶⁵ are taken together with the carbon to which they are attached to form a cyclopentyl. In some embodiments, R⁶⁴ and R⁶⁵ are taken together with the carbon to which they are attached to form a cyclohexyl. In some embodiments, R⁶³ and R⁶⁴ are taken together with the intervening atoms to form a 5- to 6-membered heterocycloalkyl, which is optionally substituted with one or more R^{64a}. In some embodiments, R⁶³ and R⁶⁴ are taken together with the intervening atoms to form a 5-membered heterocycloalkyl, which is optionally substituted with —F, C₁-C₃alkyl, C₁-C₃haloalkyl, —OH, or phenyl. In some embodiments, one of R^{64a} is a CG. In some embodiments, the CG is an azide or a terminal alkyne. In some embodiments, the CG is an azide. In some embodiments, the CG is a terminal alkyne.

In some embodiments of Formula (I), (I′), (I″), (II), (III), (IV1), (IV2), (IV3), (IV4), (IV5), or (IV6), or a pharmaceutically acceptable salt thereof, R⁶³ is hydrogen or C₁-C₃alkyl optionally substituted with one to three substituents independently selected from R^{f}. In some embodiments, R⁶³ is hydrogen. In some embodiments, R⁶³ is C₁-C₃alkyl. In some embodiments, R⁶³ is methyl.

In some embodiments of Formula (I), (I′), (I″), (II), (III), (IV1), (IV2), (IV3), (IV4), (IV5), or (IV6), or a pharmaceutically acceptable salt thereof, X1 is NMe-Hcy, NMe-Lys, or NMe-hLys; X2 is Tyr; X3 is D-Trp, or (S-βMe) D-Trp, (S-βMe)-Trp, (R-βMe) D-Trp, or (R-βMe)-Trp; X4 is PipzaA, 3-Azetidine-hAla, Lys(Me), Chg4N or Cha4N; X5 is Thr or Alt; and X6 is Phe.

The structures of exemplary cyclic peptides of the present disclosure can be found in Table 1. In some embodiments, the monocyclic peptide is a peptide of Table 1.

TABLE 1

| SEQ ID NO | X1 | X2 | X3 | X4 | X5 | X6 |
|---|---|---|---|---|---|---|
| 1 | NMe-Ala | Tyr | D-Trp | Lys | Val | Phe |
| 2 | NMe-Ala | Tyr | D-Aph(Cbm) | Lys | Val | Phe |
| 3 | NMe-Lys | Tyr | D-Trp | Lys | Val | Phe |
| 4 | NMe-dLys | Tyr | D-Trp | Lys | Val | Phe |
| 5 | NMe-Lys | Tyr | D-Trp | Lys | Thr | Phe |
| 6 | NMe-Ala | L-DOPA | D-Trp | Lys | Val | Phe |
| 7 | NMe-Ala | 3,3-diPhe | D-Trp | Lys | Val | Phe |
| 8 | NMe-Ala | Aph(Hor) | D-Trp | Lys | Val | Phe |
| 9 | NMe-Orn | Tyr | D-Trp | Lys | Val | Phe |
| 10 | NMe-Cys | Tyr | D-Trp | Lys | Val | Phe |
| 11 | NMe-Hcy | Tyr | D-Trp | Lys | Val | Phe |
| 12 | NMe-Glu | Tyr | D-Trp | Lys | Val | Phe |
| 13 | NMe-hGlu | Tyr | D-Trp | Lys | Val | Phe |
| 14 | NMe-Cys | 3Pal | D-Trp | Lys | Thr | Phe |
| 15 | NMe-Cys | 4Pal | D-Trp | Lys | Thr | Phe |
| 16 | NMe-Hcy | 3Pal | D-Trp | Lys | Thr | Phe |
| 17 | NMe-Hcy | 4Pal | D-Trp | Lys | Thr | Phe |
| 18 | NMe-Amp | Tyr | D-Trp | Lys | Thr | Phe |
| 19 | NMe-hLys | Tyr | D-Trp | Lys | Thr | Phe |
| 20 | NMe-Hcy | (S-βMe) | D-Trp | Lys | Thr | Phe |
| | | Phe | | | | |
| 21 | NMe-Hcy | (R-βMe) | D-Trp | Lys | Thr | Phe |
| | | Phe | | | | |
| 22 | NMe-Hcy | Tyr | (S-βMe) D- | Lys | Thr | Phe |
| | | | Trp | | | |
| 23 | NMe-Hcy | Tyr | D-Trp | Lys | Thr | Phe |
| 24 | NMe-Cys | Tyr | D-Trp | Lys | Thr | Phe |
| 25 | NMe-Hcy | Tyr | D-Trp | Lys | Thr | 3Pal |
| 26 | NMe-Hcy | Tyr | D-Trp | Lys | Thr | 4Pal |
| 27 | NMe-Hcy | Tyr | D-Trp | Lys | Thr | (S-βMe) |
| | | | | | | Phe |
| 28 | NMe-Hcy | Tyr | D-Trp | Lys | Thr | 3N-Tyr |
| 29 | NMe-Hcy | 5FY | D-Trp | Lys | Thr | Phe |
| 30 | NMe-Hcy | Tyr | D-Trp | Lys | Thr | (R-βMe) |
| | | | | | | Phe |
| 31 | NMe-Hcy | F3CON | D-Trp | Lys | Thr | Phe |
| 32 | NMe-Hcy | F2CON | D-Trp | Lys | Thr | Phe |
| 33 | NMe-Hcy | Tyr | D-Trp | Lys | Thr | F3CON |
| 34 | NMe-Hcy | Tyr | D-6F-Trp | Lys | Thr | Phe |
| 35 | NMe-Hcy | Tyr | D-Trp | Lys | Alt | Phe |
| 36 | NMe-Hcy | 4Pal | (S-βMe) D- | Lys | Thr | Phe |
| | | | Trp | | | |
| 37 | NMe-Hcy | 3Pal | (S-βMe) D- | Lys | Thr | Phe |
| | | | Trp | | | |
| 38 | NMe-Hcy | (R-βMe) | (S-βMe) D- | Lys | Thr | Phe |
| | | Phe | Trp | | | |
| 39 | NMe-Hcy | 4Pal | (S-βMe) D- | Lys | Thr | 3Pal |
| | | | Trp | | | |
| 40 | NMe-Hcy | 4Pal | (S-βMe) D- | Lys | Thr | (R-βMe) |
| | | | Trp | | | Phe |
| 41 | NMe-Hcy | 4Pal | (S-βMe) D- | Lys | Thr | (S-βMe) |
| | | | Trp | | | Phe |
| 42 | NMe-Hcy | 4Pal | D-Trp | Lys | Thr | 4Pal |
| 43 | NMe-Hcy | 4Pal | D-Trp | Lys | Thr | 3Pal |
| 44 | NMe-Hcy | 4Pal | D-Trp | Lys | Thr | (S-βMe) |
| | | | | | | Phe |
| 45 | NMe-Hcy | 4Pal | D-Trp | Lys | Thr | (R-βMe) |
| | | | | | | Phe |
| 46 | NMe-Hcy | 4Pal | D-Trp | Lys | Thr | 3N-Tyr |
| 47 | NMe-Hcy | Tyr | D-Trp | NMe-Lys | Thr | Phe |
| 48 | NMe-Hcy | Tyr | NMe-D-Trp | NMe-Lys | Thr | Phe |
| 48 | NMe-hHcy | Tyr | D-Trp | Lys | Thr | Phe |
| 50 | NMe-Hcy | 4-Pal | D-Trp | Lys | Thr | Phe |
| 51 | NMe-Hcy | Tyr | NMe-D-Trp | NMe-Lys | Thr | NMe-Phe |
| 52 | NMe-Hcy | Tyr | D-Trp | Lys | Cbg | Phe |
| 53 | NMe-Hcy | Tyr | (S-βMe) D- | Lys | Val | Phe |
| | | | Trp | | | |
| 54 | NMe-Hcy | Tyr | NMe-D-Trp | NMe-Lys | Val | Phe |
| 55 | NMe-Hcy | 3,3-diPhe | D-Trp | Lys | Val | Phe |
| 56 | NMe-Hcy | Tyr | D-Trp | Lys | Val | Mpd |
| 57 | NMe-Hcy | Tyr | D-Trp | Lys | Val | Cha |
| 58 | NMe-Hcy | Tyr | D-Trp | Lys | Tme | Phe |
| 59 | NMe-Hcy | Tyr | D-Trp | Lys | Cba | Phe |
| 60 | NMe-Hcy | Tyr | D-Trp | Lys | Cpg | Phe |
| 61 | NMe-Hcy | Tyr | D-Trp | Lys | Thr | Gly |
| 62 | NMe-Hcy | Tyr | D-Trp | Lys | Thr | Phg |
| 63 | NMe-Hcy | 4Pal | (S-βMe) D- | Lys | Val | Phe |
| | | | Trp | | | |
| 64 | NMe-Hcy | Tyr | D-Trp | Cha4N | Thr | Phe |
| 65 | NMe-Hcy | Tyr | D-Trp | Cba3N | Thr | Phe |
| 66 | NMe-Hcy | Tyr | D-Trp | Chg4N | Thr | Phe |
| 67 | NMe-Hcy | Tyr | D-Trp | Lys | Thr | G(cPr) |
| 68 | NMe-Hcy | Tyr | D-Trp | Lys | Thr | Asn |
| 69 | NMe-Hcy | Tyr | D-Trp | Lys | Thr | His |
| 70 | NMe-Hcy | Tyr | D-Trp | Lys | Thr | Ala |
| 71 | NMe-Hcy | Tyr | D-Trp | Lys | Thr | Ser |
| 72 | NMe-Hcy | Tyr | NMe-D-Trp | Lys | Thr | Phe |
| 73 | NMe-Hcy | Tyr | Trp | Lys | Thr | Phe |
| 74 | NMe-Hcy | Tyr | D-Trp | Lys | Thr | NMe-Phe |
| 75 | NMe-Hcy | Gly | D-Trp | Lys | Thr | Phe |
| 76 | NMe-Hcy | Ala | D-Trp | Lys | Thr | Phe |
| 77 | NMe-Dab | Tyr | D-Trp | Lys | Thr | Phe |
| 78 | NMe-Dap | Tyr | D-Trp | Lys | Thr | Phe |
| 79 | NMe-Hcy | Tyr | D-Trp | Lys | Thr | Ser(Ph) |
| 80 | NMe-Hcy | 3,5-diF Tyr | D-Trp | Lys | Thr | Phe |
| 81 | NMe-Hcy | Tyr | D-Trp | Lys | Ala | Phe |
| 82 | NMe-Hcy | Tyr | (S-βMe) D- | Cha4N | Thr | Phe |
| | | | Trp | | | |
| 83 | NMe-Hcy | Tyr | Trp | Cha4N | Thr | Phe |
| 84 | NMe-Lys | Tyr | (S-βMe) D- | Cha4N | Thr | Phe |
| | | | Trp | | | |
| 85 | NMe-Hcy | Tyr | D-Trp | Lys(Me) | Thr | Phe |
| 86 | NMe-Hcy | Tyr | D-Trp | Lys(diMe) | Thr | Phe |
| 87 | NMe-Hcy | Tyr | D-Trp | PipzaA | Thr | Phe |
| 88 | NMe-Hcy | Tyr | (S-βMe) D- | Cha4N | Thr | Ala |
| | | | Trp | | | |
| 89 | NMe-Hcy | 3,3-diPhe | D-Trp | Cha4N | Thr | Ala |
| 90 | NMe-Hcy | Tyr | D-Trp | D-Lys | Thr | Phe |
| 91 | NMe- | Tyr | D-Trp | Lys | Thr | Phe |
| | Chg4N | | | | | |
| 92 | NMe- | Tyr | D-Trp | Lys | Thr | Phe |
| | Cha4N | | | | | |
| 93 | NMe-Hcy | Tyr | 4-aza-Trp | Lys | Thr | Phe |
| 94 | NMe-Hcy | Tyr | D-Trp | Lys | Thr | Pro |
| 95 | NMe-D- | Tyr | D-Trp | Lys | Thr | Phe |
| | Hcy | | | | | |
| 96 | NMe-Asp | Tyr | D-Trp | Lys | Thr | Phe |
| 97 | NMe-Hcy | Tyr | D-Trp | (3- | Thr | Phe |
| | | | | Azetidine)- | | |
| | | | | hAla | | |
| 98 | NMe-Hcy | Tyr | D-Trp | 4-oxa Lys | Thr | Phe |
| 99 | NMe-Hcy | Tyr | 5-aza-Trp | Lys | Thr | Phe |
| 100 | NMe-Hcy | Tyr | 6-aza-Trp | Lys | Thr | Phe |
| 101 | NMe-Hcy | Tyr | 7-aza-Trp | Lys | Thr | Phe |
| 102 | NMe-Lys | Tyr | D-Trp | Cha4N | Thr | Tyr |
| 103 | NMe-Lys | Tyr | D-Trp | Cha4N | Thr | mTyr |
| 104 | NMe-Lys | Tyr | (S-βMe) D- | Cha4N | Thr | mTyr |
| | | | Trp | | | |
| 105 | NMe-Hcy | Tyr | (S-βMe) D- | 4- | Thr | Phe |
| | | | Trp | aminomethyl | | |
| | | | | Phe | | |
| 106 | NMe-Hcy | Tyr | (S-βMe) D- | Lys(iPr) | Thr | Phe |
| | | | Trp | | | |
| 107 | NMe-Hcy | Tyr | D-Trp | Ser(3- | Thr | Phe |
| | | | | azetidine) | | |
| 108 | NMe-Hcy | Tyr | D-Trp | Nva(NH- | Thr | Phe |
| | | | | NH2) | | |
| 109 | NMe-Hcy | Tyr | (R-βMe) | Lys | Thr | Phe |
| | | | Trp | | | |
| 110 | NMe-Lys | Tyr | (R-βMe) | Cha4N | Thr | Phe |
| | | | Trp | | | |
| 111 | NMe-Hcy | Tyr | (S-βMe) D- | PipzaA | Thr | Phe |
| | | | Trp | | | |
| 112 | NMe-Hcy | Tyr | (6-Me) D- | Cha4N | Thr | Phe |
| | | | Trp | | | |
| 113 | NMe-Hcy | Tyr | (5-Me) D- | Cha4N | Thr | Phe |
| | | | Trp | | | |
| 114 | NMe-Hcy | Tyr | (5-Cl) D- | Cha4N | Thr | Phe |
| | | | Trp | | | |
| 115 | NMe-Hcy | Tyr | (5-MeO) D- | Cha4N | Thr | Phe |
| | | | Trp | | | |
| 116 | 4-oxa | Tyr | (S-βMe) D- | Cha4N | Thr | Phe |
| | NMe-Lys | | Trp | | | |
| 117 | NMe-Lys | Tyr | (S-βMe) D- | (3- | Thr | Phe |
| | | | Trp | Azetidine)- | | |
| | | | | hAla | | |
| 118 | NMe-Lys | 4MeO-Phe | (S-βMe) D- | Cha4N | Thr | Phe |
| | | | Trp | | | |
| 119 | NMe-Dap | Tyr | (S-βMe) D- | Cha4N | Thr | Phe |
| | | | Trp | | | |
| 120 | NMe-Lys | Tyr | (S-βMe) D- | Cha4N | Thr | Cha |
| | | | Trp | | | |
| 121 | NMe-Lys | Tyr | (S-βMe) D- | Cha4N | Thr | Ser |
| | | | Trp | | | |
| 122 | NMe-Lys | Tyr | (S-βMe) D- | Cha4N | Thr | 4Pal |
| | | | Trp | | | |
| 123 | NMe-Lys | Tyr | (S-βMe) D- | Lys(Me) | Thr | Phe |
| | | | Trp | | | |
| 124 | NMe-Lys | Tyr | (S-βMe) D- | PipzaA | Thr | Phe |
| | | | Trp | | | |
| 125 | NMe-Hcy | 3MeO-Phe | (S-βMe) D- | Cha4N | Thr | Phe |
| | | | Trp | | | |
| 126 | NMe-Lys | Tyr | D-Tpi | Cha4N | Thr | Phe |
| 127 | NMe-Lys | Tyr | (S-βMe) D- | azaLys | Thr | Phe |
| | | | Trp | | | |
| 128 | NMe-Hcy | Tyr | (7-Me) D- | Cha4N | Thr | Phe |
| | | | Trp | | | |
| 129 | NMe-Lys | Tyr(Phe) | (S-βMe) D- | Cha4N | Thr | Phe |
| | | | Trp | | | |
| 130 | NMe-Lys | 3,3-diPhe | (S-βMe) D- | Cha4N | Thr | Phe |
| | | | Trp | | | |
| 131 | NMe-Hcy | Tyr | D-Trp | (D/L)Hly | Thr | Phe |
| 132 | NMe-Lys | Tyr | (S-βMe) D- | Cha4N | Ala | Phe |
| | | | Trp | | | |
| 133 | NMe-Hcy | Tyr | Trp | (D/L)Hly | Thr | Phe |
| 134 | NMe-Lys | Tyr | (S-βMe) D- | Cha4NH2 | Thr | Phe |
| | | | Trp | | | |
| 135 | Nlys | Tyr | (S-βMe) D- | Cha4N | Thr | Phe |
| | | | Trp | | | |
| 136 | trans-Hyp | D-Phg | (S-βMe) D- | Cha4N | Tyr(OBn) | Phe |
| | | | Trp | | | |
| 137 | trans-Hyp | Tyr | (S-βMe) D- | Cha4N | Tyr(OBn) | Phe |
| | | | Trp | | | |
| 138 | trans-Hyp | Tyr | (S-βMe) D- | Cha4N | Thr | Phe |
| | | | Trp | | | |
| 139 | NMe-hLys | Tyr | (S-βMe) D- | Cha4N | Thr | Phe |
| | | | Trp | | | |
| 140 | NMe-Lys | Tyr | 2-aza-(D- | Cha4N | Thr | Phe |
| | | | Trp) | | | |
| 141 | NMe- | Tyr | (S-βMe) D- | Cha4N | Thr | Phe |
| | Lys(Me) | | Trp | | | |

The structures of exemplary cyclic peptides and conjugates of the present disclosure can be found in Table 2.

TABLE 2

| SEQ ID | Conjugate | Side chain | Conjugation | Conjugation | | | | | | |
|---|---|---|---|---|---|---|---|---|---|---|
| NO | ID | conjugation | Linker B | Linker A | X1 | X2 | X3 | X4 | X5 | X6 |
| 1 | RB- | | | | NMe-Ala | Tyr | D-Trp | Lys | Val | Phe |
| | 0002327 | | | | | | | | | |
| 2 | RB- | | | | NMe-Ala | Tyr | D- | Lys | Val | Phe |
| | 0002328 | | | | | | Aph(Cbm) | | | |
| 142 | RB- | Ac | | | NMe-Lys | Tyr | D-Trp | Lys | Val | Phe |
| | 0002329 | | | | | | | | | |
| 143 | RB- | Ac | | | NMe-dLys | Tyr | D-Trp | Lys | Val | Phe |
| | 0002593 | | | | | | | | | |
| 144 | RB- | Ac | | | NMe-Lys | Tyr | D-Trp | Lys | Thr | Phe |
| | 0002594 | | | | | | | | | |
| 6 | RB- | | | | NMe-Ala | L-DOPA | D-Trp | Lys | Val | Phe |
| | 0002595 | | | | | | | | | |
| 7 | RB- | | | | NMe-Ala | 3,3-diPhe | D-Trp | Lys | Val | Phe |
| | 0002596 | | | | | | | | | |
| 8 | RB- | | | | NMe-Ala | Aph(Hor) | D-Trp | Lys | Val | Phe |
| | 0002597 | | | | | | | | | |
| 145 | RB- | DOTA-Lu | Ahx | | NMe-Lys | Tyr | D-Trp | Lys | Val | Phe |
| | 0002670 | | | | | | | | | |
| 146 | RB- | DOTA-Lu | Ahx | | NMe-Orn | Tyr | D-Trp | Lys | Val | Phe |
| | 0002672 | | | | | | | | | |
| 147 | RB- | DOTA-Lu | Dah | COCH2 | NMe-Cys | Tyr | D-Trp | Lys | Val | Phe |
| | 0002674 | | | | | | | | | |
| 148 | RB- | DOTA-Lu | Dah | COCH2 | NMe-Hcy | Tyr | D-Trp | Lys | Val | Phe |
| | 0002676 | | | | | | | | | |
| 149 | RB- | DOTA-Lu | Dah | | NMe-Glu | Tyr | D-Trp | Lys | Val | Phe |
| | 0002678 | | | | | | | | | |
| 150 | RB- | DOTA-Lu | Dah | | NMe-hGlu | Tyr | D-Trp | Lys | Val | Phe |
| | 0002680 | | | | | | | | | |
| 151 | RB- | DOTA-Lu | Dah | COCH2 | NMe-Cys | 3Pal | D-Trp | Lys | Thr | Phe |
| | 0002946 | | | | | | | | | |
| 152 | RB- | DOTA-Lu | Dah | COCH2 | NMe-Cys | 4Pal | D-Trp | Lys | Thr | Phe |
| | 0002947 | | | | | | | | | |
| 153 | RB- | DOTA-Lu | Dah | COCH2 | NMe-Hcy | 3Pal | D-Trp | Lys | Thr | Phe |
| | 0003000 | | | | | | | | | |
| 154 | RB- | DOTA-Lu | Dah | COCH2 | NMe-Hcy | 4Pal | D-Trp | Lys | Thr | Phe |
| | 0003002 | | | | | | | | | |
| 155 | RB- | DOTA-Lu | Dah | | NMe-Amp | Tyr | D-Trp | Lys | Thr | Phe |
| | 0003055 | | | | | | | | | |
| 156 | RB- | DOTA-Lu | Ahx | | NMe-hLys | Ty | D-Trp | Lys | Thr | Phe |
| | 0003057 | | | | | | | | | |
| 157 | RB- | DOTA-Lu | Dah | COCH2 | NMe-Hcy | (S-βMe) | D-Trp | Lys | Thr | Phe |
| | 0003136 | | | | | Phe | | | | |
| 158 | RB- | DOTA-Lu | Dah | COCH2 | NMe-Hcy | (R-βMe) | D-Trp | Lys | Thr | Phe |
| | 0003137 | | | | | Phe | | | | |
| 159 | RB- | DOTA-Lu | Dah | COCH2 | NMe-Hcy | Tyr | (S-βMe) | Lys | Thr | Phe |
| | 0003148 | | | | | | D-Trp | | | |
| 160 | RB- | DOTA-Lu | Dah | COCH2 | NMe-Hcy | Tyr | D-Trp | Lys | Thr | Phe |
| | 0003149 | | | | | | | | | |
| 161 | RB- | DOTA-Lu | Dah | COCH2 | NMe-Cys | Ty | D-Trp | Lys | Thr | Phe |
| | 0003164 | | | | | | | | | |
| 162 | RB- | DOTA-Lu | TXA | | NMe-Lys | Tyr | D-Trp | Lys | Thr | Phe |
| | 0003246 | | | | | | | | | |
| 163 | RB- | DOTA-Lu | Aeea | | NMe-Lys | Tyr | D-Trp | Lys | Thr | Phe |
| | 0003248 | | | | | | | | | |
| 164 | RB- | DOTA-Lu | Dah | COCH2 | NMe-Hcy | Tyr | D-Trp | Lys | Thr | 3Pal |
| | 0003276 | | | | | | | | | |
| 165 | RB- | DOTA-Lu | Dah | COCH2 | NMe-Hcy | Tyr | D-Trp | Lys | Thr | 4Pal |
| | 0003277 | | | | | | | | | |
| 166 | RB- | DOTA-Lu | Dah | COCH2 | NMe-Hcy | Tyr | D-Trp | Lys | Thr | (S-βMe) |
| | 0003278 | | | | | | | | | Phe |
| 167 | RB- | DOTA-Lu | Dal | COCH2 | NMe-Hcy | Tyr | D-Trp | Lys | Thr | 3N-Tyr |
| | 0003279 | | | | | | | | | |
| 168 | RB- | DOTA-Lu | Dah | COCH2 | NMe-Hcy | 5FY | D-Trp | Lys | Thr | Phe |
| | 0003280 | | | | | | | | | |
| 169 | RB- | DOTA-Lu | Dah | COCH2 | NMe-Hcy | Tyr | D-Trp | Lys | Thr | (R-βMe) |
| | 0003281 | | | | | | | | | Phe |
| 170 | RB- | DOTA-Lu | | | NMe-Lys | Tyr | D-Trp | Lys | Thr | Phe |
| | 0003292 | | | | | | | | | |
| 171 | RB- | DOTA-Lu | Acea | | NMe-Lys | Tyr | D-Trp | Lys | Thr | Phe |
| | 0003294 | | | | | | | | | |
| 172 | RB- | DOTA-Lu | Aoc | | NMe-Lys | Tyr | D-Trp | Lys | Thr | Phe |
| | 0003296 | | | | | | | | | |
| 173 | RB- | DOTA-Lu | | Asu | NMe-Lys | Tyr | D-Trp | Lys | Thr | Phe |
| | 0003298 | | | | | | | | | |
| 174 | RB- | DOTA-Lu | Dah | COCH2 | NMe-Hcy | F3CON | D-Trp | Lys | Thr | Phe |
| | 0003302 | | | | | | | | | |
| 175 | RB- | DOTA-Lu | Dah | COCH2 | NMe-Hcy | F2CON | D-Trp | Lys | Thr | Phe |
| | 0003304 | | | | | | | | | |
| 176 | RB- | DOTA-Lu | Dah | COCH2 | NMe-Hcy | Tyr | D-Trp | Lys | Thr | F3CON |
| | 0003306 | | | | | | | | | |
| 177 | RB- | DOTA-Lu | Dah | COCH2 | NMe-Hcy | Tyr | D-6F-Trp | Lys | Thr | Phe |
| | 0003309 | | | | | | | | | |
| 178 | RB- | DOTA-Lu | Peg3 | COCH2 | NMe-Hcy | Tyr | D-Trp | Lys | Alt | Phe |
| | 0003357 | | | | | | | | | |
| 179 | RB- | DOTA-Lu | Dah | COCH2CH2 | NMe-Hcy | Tyr | D-Trp | Lys | Val | Phe |
| | 0003374 | | | | | | | | | |
| 180 | RB- | DOTA-Lu | Hly | COCH2CH2 | NMe-Hcy | Tyr | D-Trp | Lys | Val | Phe |
| | 0003375 | | | | | | | | | |
| 181 | RB- | DOTA-Lu | Dah | COCH2 | NMe-Hcy | 4Pal | (S-βMe) | Lys | Thr | Phe |
| | 0003381 | | | | | | D-Trp | | | |
| 182 | RB- | DOTA-Lu | Dah | COCH2 | NMe-Hcy | 3Pal | (S-βMe) | Lys | Thr | Phe |
| | 0003382 | | | | | | D-Trp | | | |
| 183 | RB- | DOTA-Lu | Dah | COCH2 | NMe-Hcy | (R-βMe) | (S-βMe) | Lys | Thr | Phe |
| | 0003383 | | | | | Phe | D-Trp | | | |
| 184 | RB- | DOTA | Dah | COCH2 | NMe-Hcy | 4Pal | (S-βMe) | Lys | Thr | 3Pal |
| | 0003407 | | | | | | D-Trp | | | |
| 185 | RB- | DOTA | Dah | COCH2 | NMe-Hcy | 4Pal | (S-βMe) | Lys | Thr | (R-βMe) |
| | 0003408 | | | | | | D-Trp | | | Phe |
| 186 | RB- | DOTA | Dah | COCH2 | NMe-Hcy | 4Pal | (S-βMe) | Lys | Thr | (S-βMe) |
| | 0003409 | | | | | | D-Trp | | | Phe |
| 187 | RB- | DOTA-Lu | Dah | COCH2 | NMe-Hcy | 4Pal | D-Trp | Lys | Thr | 4Pal |
| | 0003443 | | | | | | | | | |
| 188 | RB- | DOTA-Lu | Dah | COCH2 | NMe-Hcy | 4Pal | D-Trp | Lys | Thr | 3Pal |
| | 0003445 | | | | | | | | | |
| 189 | RB- | DOTA-Lu | Dah | COCH2 | NMe-Hcy | 4Pal | D-Trp | Lys | Thr | (S-βMe) |
| | 0003447 | | | | | | | | | Phe |
| 190 | RB- | DOTA-Lu | Dah | COCH2 | NMe-Hcy | 4Pal | D-Trp | Lys | Thr | (R-βMe) |
| | 0003449 | | | | | | | | | Phe |
| 191 | RB- | DOTA-Lu | Dah | COCH2 | NMe-Hcy | 4Pal | D-Trp | Lys | Thr | 3N-Tyr |
| | 0003451 | | | | | | | | | |
| 192 | RB- | DOTA-Lu | Dah | COCH2 | NMe-Hcy | Tyr | D-Trp | NMe-Lys | Thr | Phe |
| | 0003453 | | | | | | | | | |
| 193 | RB- | DOTA-Lu | Dah | COCH2 | NMe-Hcy | Ty | NMe-D- | NMe-Lys | Thr | Phe |
| | 0003455 | | | | | | Trp | | | |
| 194 | RB- | DOTA-Lu | Dah | COCH2 | NMe-Hcy | Tyr | D-Trp | Lys | Alt | Phe |
| | 0003464 | | | | | | | | | |
| 195 | RB- | DOTA-Lu | Dap | COCH2 | NMe-Hcy | Tyr | D-Trp | Lys | Thr | Phe |
| | 0003482 | | | | | | | | | |
| 196 | RB- | DOTA-Lu | Dah | COCH2 | NMe-hHcy | Ty | D-Trp | Lys | Thr | Phe |
| | 0003484 | | | | | | | | | |
| 197 | RB- | DOTA-Lu | Daop | COCH2 | NMe-Hcy | Tyr | D-Trp | Lys | Thr | Phe |
| | 0003486 | | | | | | | | | |
| 198 | RB- | DOTA-Lu | Peg3 | COCH2 | NMe-Hcy | 4-Pal | D-Trp | Lys | Thr | Phe |
| | 0003504 | | | | | | | | | |
| 199 | RB- | DOTA-Lu | | | NMe-HLys | Tyr | D-Trp | Lys | Thr | Phe |
| | 0004536 | | | | | | | | | |
| 200 | RB- | DOTA-Lu | Dah | COCH2 | NMe-Hcy | Tyr | NMe-D- | NMe-Lys | Thr | NMe- |
| | 0004538 | | | | | | Trp | | | Phe |
| 201 | RB- | DOTA-Lu | Dah | COCH2 | NMe-Hcy | Tyr | D-Trp | Lys | Cbg | Phe |
| | 0004540 | | | | | | | | | |
| 202 | RB- | DOTA-Lu | Dah | COCH2 | NMe-Hcy | Tyr | (S-βMe) | Lys | Val | Phe |
| | 0004609 | | | | | | D-Trp | | | |
| 203 | RB- | DOTA-Lu | Dah | COCH2 | NMe-Hcy | Tyr | NMe-D- | NMe-Lys | Val | Phe |
| | 0004611 | | | | | | Trp | | | |
| 204 | RB- | DOTA-Lu | Dah | COCH2 | NMe-Hcy | 3,3-diPhe | D-Trp | Lys | Val | Phe |
| | 0004613 | | | | | | | | | |
| 205 | RB- | DOTA-Lu | Dah | COCH2 | NMe-Hcy | Tyr | D-Trp | Lys | Val | Mpd |
| | 0004615 | | | | | | | | | |
| 206 | RB- | DOTA-Lu | Dah | COCH2 | NMe-Hcy | Tyr | D-Trp | Lys | Val | Cha |
| | 0004617 | | | | | | | | | |
| 207 | RB- | DOTA-Lu | Dah | COCH2 | NMe-Hcy | Tyr | D-Trp | Lys | Tme | Phe |
| | 0004619 | | | | | | | | | |
| 208 | RB- | DOTA-Lu | Dah | COCH2 | NMe-Hcy | Tyr | D-Trp | Lys | Cba | Phe |
| | 0004621 | | | | | | | | | |
| 209 | RB- | DOTA-Lu | Dah | COCH2 | NMe-Hcy | Tyr | D-Trp | Lys | Cpg | Phe |
| | 0004623 | | | | | | | | | |
| 210 | RB- | DOTA-Lu | Dah | COCH2 | NMe-Hcy | Ty | D-Trp | Lys | Thr | Gly |
| | 0004819 | | | | | | | | | |
| 211 | RB- | DOTA-Lu | Dah | COCH2 | NMe-Hcy | Ty | D-Trp | Lys | Thr | Phg |
| | 0004821 | | | | | | | | | |
| 212 | RB- | DOTA-Lu | Peg3 | COCH2 | NMe-Hcy | Tyr | D-Trp | Lys | Val | Phe |
| | 0004823 | | | | | | | | | |
| 213 | RB- | DOTA-Lu | Dah | COCH2 | NMe-Hcy | 4Pal | (S-βMe) | Lys | Val | Phe |
| | 0004825 | | | | | | D-Trp | | | |
| 214 | RB- | DOTA-Lu | Dah | COCH2 | NMe-Hcy | Tyr | D-Trp | Cha4N | Thr | Phe |
| | 0004864 | | | | | | | | | |
| 215 | RB- | DOTA-Lu | Dah | COCH2 | NMe-Hcy | Tyr | D-Trp | Cba3N | Thr | Phe |
| | 0004866 | | | | | | | | | |
| 216 | RB- | DOTA-Lu | Dah | COCH2 | NMe-Hcy | Tyr | D-Trp | Chg4N | Thr | Phe |
| | 0004868 | | | | | | | | | |
| 217 | RB- | DOTA-Lu | Dah | COCH2 | NMe-Hcy | Tyr | D-Trp | Lys | Thr | G(cPr) |
| | 0005247 | | | | | | | | | |
| 218 | RB- | DOTA-Lu | Dah | COCH2 | NMe-Hcy | Tyr | D-Trp | Lys | Thr | Asn |
| | 0005253 | | | | | | | | | |
| 219 | RB- | DOTA-Lu | Dah | COCH2 | NMe-Hcy | Tyr | D-Trp | Lys | Thr | His |
| | 0005255 | | | | | | | | | |
| 220 | RB- | DOTA-Lu | Dah | COCH2 | NMe-Hcy | Tyr | D-Trp | Lys | Thr | Ala |
| | 0005266 | | | | | | | | | |
| 221 | RB- | DOTA-Lu | Dah | COCH2 | NMe-Hcy | Tyr | D-Trp | Lys | Thr | Ser |
| | 0005270 | | | | | | | | | |
| 222 | RB- | DOTA-Lu | PEG2 | COCH2 | NMe-Hcy | Tyr | D-Trp | Lys | Thr | Phe |
| | 0005272 | | | | | | | | | |
| 223 | RB- | DOTAGA- | Dah | COCH2 | NMe-Hcy | Tyr | D-Trp | Lys | Thr | Phe |
| | 0005274 | Lu | | | | | | | | |
| 224 | RB- | DOTA-Lu | dK | COCH2 | NMe-Hcy | Tyr | D-Trp | Lys | Thr | Phe |
| | 0005276 | | | | | | | | | |
| 225 | RB- | DOTA-Lu | gamma dK | COCH2 | NMe-Hcy | Tyr | D-Trp | Lys | Thr | Phe |
| | 0005278 | | | | | | | | | |
| 226 | RB- | DOTA-Lu | PEG3 | COCH2 | NMe-Hcy | Tyr | D-Trp | Lys | Thr | Phe |
| | 0005284 | | | | | | | | | |
| 227 | RB- | DOTA-Lu | PEG3 | COCH2 | NMe-Hcy | Tyr | D-Trp | Lys | Thr | Gly |
| | 0005286 | | | | | | | | | |
| 228 | RB- | DOTA-Lu | Dah | COCH2 | NMe-Hcy | Tyr | NMe-D- | Lys | Thr | Phe |
| | 0005330 | | | | | | Trp | | | |
| 229 | RB- | DOTA-Lu | Peg3 | COCH2 | NMe-Hcy | Tyr | Trp | Lys | Thr | Phe |
| | 0005502 | | | | | | | | | |
| 230 | RB- | DOTA-Lu | Dah | COCH2 | NMe-Hcy | Tyr | D-Trp | Lys | Thr | NMe- |
| | 0005692 | | | | | | | | | Phe |
| 231 | RB- | DOTA-Lu | Dah | COCH2 | NMe-Hcy | Gly | D-Trp | Lys | Thr | Phe |
| | 0005694 | | | | | | | | | |
| 232 | RB- | DOTA-Lu | Dah | COCH2 | NMe-Hcy | Ala | D-Trp | Lys | Thr | Phe |
| | 0005696 | | | | | | | | | |
| 233 | RB- | DOTA-Lu | Ahx | | NMe-Dab | Tyr | D-Trp | Lys | Thr | Phe |
| | 0005717 | | | | | | | | | |
| 234 | RB- | DOTA-Lu | Ahx | | NMe-Dap | Tyr | D-Trp | Lys | Thr | Phe |
| | 0005728 | | | | | | | | | |
| 235 | RB- | DOTA-Lu | Ahx | Asp | NMe-Dap | Tyr | D-Trp | Lys | Thr | Phe |
| | 0005730 | | | | | | | | | |
| 236 | RB- | DOTA-Lu | Ahx | Beta-Asp | NMe-Dap | Tyr | D-Trp | Lys | Thr | Phe |
| | 0005732 | | | | | | | | | |
| 237 | RB- | DOTA-Lu | Ahx | D-Asp | NMe-Dap | Tyr | D-Trp | Lys | Thr | Phe |
| | 0005734 | | | | | | | | | |
| 238 | RB- | DOTA-Lu | Dah | Beta D-Asp | NMe-Dap | Tyr | D-Trp | Lys | Thr | Phe |
| | 0005736 | | | | | | | | | |
| 239 | RB- | DOTA-Lu | Peg3 | COCH2 | NMe-Hcy | Tyr | D-Trp | Lys | Thr | Ser(Ph) |
| | 0005738 | | | | | | | | | |
| 240 | RB- | DOTA-Lu | Dah | COCH2 | NMe-Hcy | 3,5-diF | D-Trp | Lys | Thr | Phe |
| | 0005740 | | | | | Tyr | | | | |
| 241 | RB- | DOTA-Lu | Dah | COCH2 | NMe-Hcy | Tyr | D-Trp | Lys | Ala | Phe |
| | 0005744 | | | | | | | | | |
| 242 | RB- | DOTA-Lu | Dah | COCH2 | NMe-Hcy | Tyr | (S-BMe) | Cha4N | Thr | Phe |
| | 0005760 | | | | | | D-Trp | | | |
| 243 | RB- | DOTA-Lu | Dah | COCH2 | NMe-Hcy | Tyr | Trp | Cha4N | Thr | Phe |
| | 0005761 | | | | | | | | | |
| 244 | RB- | DOTA-Lu | | | NMe-Lys | Tyr | (S-βMe) | Cha4N | Thr | Phe |
| | 0005797 | | | | | | D-Trp | | | |
| 245 | RB- | DOTA-Lu | Dah | COCH2 | NMe-Hcy | Tyr | D-Trp | Lys(Me) | Thr | Phe |
| | 0005799 | | | | | | | | | |
| 246 | RB- | DOTA-Lu | Dah | COCH2 | NMe-Hcy | Tyr | D-Trp | Lys(diMe) | Thr | Phe |
| | 0005801 | | | | | | | | | |
| 247 | RB- | DOTA-Lu | Dah | COCH2 | NMe-Hcy | Tyr | D-Trp | PipzaA | Thr | Phe |
| | 0005805 | | | | | | | | | |
| 248 | RB- | DOTA-Lu | Dah | COCH2 | NMe-Hcy | Tyr | (S-βMe) | Cha4N | Thr | Ala |
| | 0005807 | | | | | | D-Trp | | | |
| 249 | RB- | DOTA-Lu | Dah | COCH2 | NMe-Hcy | 3,3-diPhe | D-Trp | Cha4N | Thr | Ala |
| | 0005809 | | | | | | | | | |
| 250 | RB- | DOTA-Lu | Dah | COCH2 | NMe-Hcy | Tyr | D-Trp | D-Lys | Thr | Phe |
| | 0005827 | | | | | | | | | |
| 251 | RB- | DOTA-Lu | Ahx | | NMe-Chg4N | Tyr | D-Trp | Lys | Thr | Phe |
| | 0005852 | | | | | | | | | |
| 252 | RB- | DOTA-Lu | Ahx | | NMe-Cha4N | Tyr | D-Trp | Lys | Thr | Phe |
| | 0005854 | | | | | | | | | |
| 253 | RB- | DOTA-Lu | Dah | COCH2 | NMe-Hcy | Tyr | 4-aza-Trp | Lys | Thr | Phe |
| | 0005858 | | | | | | | | | |
| 254 | RB- | DOTA-Lu | Dah | COCH2 | NMe-Hcy | Tyr | D-Trp | Lys | Thr | Pro |
| | 0005864 | | | | | | | | | |
| 255 | RB- | DOTA-Lu | Dah | COCH2 | NMe-D-Hcy | Tyr | D-Trp | Lys | Thr | Phe |
| | 0005866 | | | | | | | | | |
| 256 | RB- | DOTA-Lu | Dah | | NMe-Asp | Tyr | D-Trp | Lys | Thr | Phe |
| | 0005895 | | | | | | | | | |
| 257 | RB- | DOTA-Lu | Dah | COCH2 | NMe-Hcy | Tyr | D-Trp | 3-Azetidine- | Thr | Phe |
| | 0005897 | | | | | | | hAla | | |
| 258 | RB- | DOTA-Lu | Dah | COCH2 | NMe-Hcy | Tyr | D-Trp | 4-oxa Lys | Thr | Phe |
| | 0005899 | | | | | | | | | |
| 259 | RB- | DOTA-Lu | Dah | COCH2 | NMe-Hcy | Tyr | 5-aza-Trp | Lys | Thr | Phe |
| | 0005903 | | | | | | | | | |
| 260 | RB- | DOTA-Lu | Dah | COCH2 | NMe-Hcy | Tyr | 6-aza-Trp | Lys | Thr | Phe |
| | 0005905 | | | | | | | | | |
| 261 | RB- | DOTA-Lu | Dah | COCH2 | NMe-Hcy | Tyr | 7-aza-Trp | Lys | Thr | Phe |
| | 0005907 | | | | | | | | | |
| 262 | RB- | DOTA-Lu | | | NMe-Lys | Tyr | D-Trp | Cha4N | Thr | Tyr |
| | 0005983 | | | | | | | | | |
| 263 | RB- | DOTA-Lu | | | NMe-Lys | Tyr | D-Trp | Cha4N | Thr | mTyr |
| | 0005984 | | | | | | | | | |
| 264 | RB- | DOTA-Lu | | | NMe-Lys | Tyr | (S-βMe) | Cha4N | Thr | mTyr |
| | 0005985 | | | | | | D-Trp | | | |
| 265 | RB | DOTA-Lu | en | COCH2 | NMe-Hcy | Tyr | (S-βMe) | Cha4N | Thr | Phe |
| | 0005990 | | | | | | D-Trp | | | |
| 266 | RB- | DOTA-Lu | PEG3 | COCH2 | NMe-Hcy | Tyr | Trp | Cha4N | Thr | Phe |
| | 0005992 | | | | | | | | | |
| 267 | RB- | DOTA-Lu | PEG3 | COCH2 | NMe-Hcy | Tyr | (S-βMe) | Cha4N | Thr | Phe |
| | 0005994 | | | | | | D-Trp | | | |
| 268 | RB- | DOTA-Lu | Dah | COCH2 | NMe-Hcy | Tyr | (S-βMe) | 4- | Thr | Phe |
| | 0006009 | | | | | | D-Trp | aminomethyl | | |
| | | | | | | | | Phe | | |
| 269 | RB- | DOTA-Lu | Dah | COCH2 | NMe-Hcy | Tyr | (S-βMe) | Lys(iPr) | Thr | Phe |
| | 0006011 | | | | | | D-Trp | | | |
| 270 | RB- | DOTA-Lu | Dah | COCH2 | NMe-Hcy | Tyr | D-Trp | Ser(3- | Thr | Phe |
| | 0006013 | | | | | | | azetidine) | | |
| 271 | RB- | DOTA-Lu | Dah | COCH2 | NMe-Hcy | Tyr | D-Trp | Nva(NH- | Thr | Phe |
| | 0006015 | | | | | | | NH2) | | |
| 272 | RB- | DOTA-Lu | Dah | COCH2 | NMe-Hcy | Tyr | (R-βMe) | Lys | Thr | Phe |
| | 0006019 | | | | | | Trp | | | |
| 273 | RB- | DOTA-Lu | | | NMe-Lys | Tyr | (R-βMe) | Cha4N | Thr | Phe |
| | 0006069 | | | | | | Trp | | | |
| 274 | RB- | DOTA-Lu | Dah | COCH2 | NMe-Hcy | Tyr | (S-βMe) | PipzaA | Thr | Phe |
| | 0006072 | | | | | | D-Trp | | | |
| 275 | RB- | DOTA-Lu | Dah | COCH2 | NMe-Hcy | Tyr | (6-Me) D- | Cha4N | Thr | Phe |
| | 0006073 | | | | | | Trp | | | |
| 276 | RB- | DOTA-Lu | Dah | COCH2 | NMe-Hcy | Tyr | (5-Me) D- | Cha4N | Thr | Phe |
| | 0006074 | | | | | | Trp | | | |
| 277 | RB- | DOTA-Lu | Dah | COCH2 | NMe-Hcy | Tyr | (5-Cl) D- | Cha4N | Thr | Phe |
| | 0006075 | | | | | | Trp | | | |
| 278 | RB- | DOTA-Lu | Dah | COCH2 | NMe-Hcy | Tyr | (5-MeO) | Cha4N | Thr | Phe |
| | 0006076 | | | | | | D-Trp | | | |
| 279 | RB- | DOTA-Lu | | | 4-oxa NMe- | Tyr | (S-βMe) | Cha4N | Thr | Phe |
| | 0006095 | | | | Lys | | D-Trp | | | |
| 280 | RB- | DOTA-Lu | | | NMe-Lys | Tyr | (S-βMe) | 3-Azetidine- | Thr | Phe |
| | 0006128 | | | | | | D-Trp | hAla | | |
| 281 | RB- | DOTA-Lu | | | NMe-Lys | 4MeO- | (S-βMe) | Cha4N | Thr | Phe |
| | 0006139 | | | | | Phe | D-Trp | | | |
| 282 | RB- | DOTA-Lu | Ahx | Asp | NMe-Dap | Tyr | (S-βMe) | Cha4N | Thr | Phe |
| | 0006158 | | | | | | D-Trp | | | |
| 283 | RB- | DOTA-Lu | | Asp | NMe-Dap | Tyr | (S-βMe) | Cha4N | Thr | Phe |
| | 0006160 | | | | | | D-Trp | | | |
| 284 | RB- | DOTA-Lu | | | NMe-Lys | Tyr | (S-βMe) | Cha4N | Thr | Cha |
| | 0006162 | | | | | | D-Trp | | | |
| 285 | RB- | DOTA-Lu | | | NMe-Lys | Tyr | (S-βMe) | Cha4N | Thr | Ser |
| | 0006166 | | | | | | D-Trp | | | |
| 286 | RB- | DOTA-Lu | | | NMe-Lys | Tyr | (S-βMe) | Cha4N | Thr | 4Pal |
| | 0006170 | | | | | | D-Trp | | | |
| 287 | RB- | DOTA-Lu | | | NMe-Lys | Tyr | (S-βMe) | Lys(Me) | Thr | Phe |
| | 0006174 | | | | | | D-Trp | | | |
| 288 | RB- | DOTA-Lu | | | NMe-Lys | Tyr | (S-βMe) | PipzaA | Thr | Phe |
| | 0006176 | | | | | | D-Trp | | | |
| 289 | RB- | DOTA-Lu | Dah | COCH2 | NMe-Hcy | Tyr | (5-C1) D- | Cha4N | Thr | Phe |
| | 0006189 | | | | | | Trp | | | |
| 290 | RB- | DOTA-Lu | Dah | COCH2 | NMe-Hcy | 3MeO- | (S-βMe) | Cha4N | Thr | Phe |
| | 0006209 | | | | | Phe | D-Trp | | | |
| 291 | RB- | DOTA-Lu | | | NMe-Lys | Tyr | D-Tpi | Cha4N | Thr | Phe |
| | 0006213 | | | | | | | | | |
| 292 | RB- | DOTA-Lu | | Glu | NMe-Dap | Tyr | (S-βMe) | Cha4N | Thr | Phe |
| | 0006215 | | | | | | D-Trp | | | |
| 293 | RB- | DOTA-Lu | | | NMe-Lys | Tyr | (S-βMe) | azaLys | Thr | Phe |
| | 0006217 | | | | | | D-Trp | | | |
| 294 | RB- | DOTA-Lu | Dah | COCH2 | NMe-Hcy | Tyr | (7-Me) D- | Cha4N | Thr | Phe |
| | 0006219 | | | | | | Trp | | | |
| 295 | RB- | DOTA-Lu | | | NMe-Lys | Tyr(Phe) | (S-βMe) | Cha4N | Thr | Phe |
| | 0006237 | | | | | | D-Trp | | | |
| 296 | RB- | DOTA-Lu | | | NMe-Lys | 3,3-diPhe | (S-βMe) | Cha4N | Thr | Phe |
| | 0006239 | | | | | | D-Trp | | | |
| 297 | RB- | (R/S)- | | | NMe-Lys | Tyr | (S-βMe) | Cha4N | Thr | Phe |
| | 0006285 | NODAGA- | | | | | D-Trp | | | |
| | | Ga | | | | | | | | |
| 298 | RB- | NOTA-Ga | | | NMe-Lys | Tyr | (S-βMe) | Cha4N | Thr | Phe |
| | 0006286 | | | | | | D-Trp | | | |
| 299 | RB- | DOTA-Lu | Dah | COCH2 | NMe-Hcy | Tyr | D-Trp | (D/L)Hly | Thr | Phe |
| | 0006317 | | | | | | | | | |
| 300 | RB- | DOTA-Lu | | | NMe-Lys | Tyr | (S-βMe) | Cha4N | Ala | Phe |
| | 0006319 | | | | | | D-Trp | | | |
| 301 | RB- | DOTA-Lu | Dah | COCH2 | NMe-Hcy | Tyr | Trp | (D/L)Hly | Thr | Phe |
| | 0006385 | | | | | | | | | |
| 302 | RB- | DOTA-Lu | | | NMe-Lys | Tyr | (S-βMe) | Cha4NH2 | Thr | Phe |
| | 0006387 | | | | | | D-Trp | | | |
| 303 | RB- | DOTA-Lu | | | Nlys | Ty | (S-βMe) | Cha4N | Thr | Phe |
| | 0006389 | | | | | | D-Trp | | | |
| 304 | RB- | DOTA-Lu | CDAE | | trans-Hyp | D-Phg | (S-βMe) | Cha4N | Tyr(OBn) | Phe |
| | 0006391 | | | | | | D-Trp | | | |
| 305 | RB- | DOTA-Lu | CDAE | | trans-Hyp | Ty | (S-βMe) | Cha4N | Tyr(OBn) | Phe |
| | 0006393 | | | | | | D-Trp | | | |
| 306 | RB- | DOTA-Lu | CDAE | | trans-Hyp | Tyr | (S-βMe) | Cha4N | Thr | Phe |
| | 0006395 | | | | | | D-Trp | | | |
| 307 | RB- | DOTA-Lu | Gly | | NMe-Lys | Ty | (S-βMe) | Cha4N | Thr | Phe |
| | 0006397 | | | | | | D-Trp | | | |
| 308 | RB- | DOTA-Lu | bAla | | NMe-Lys | Tyr | (S-βMe) | Cha4N | Thr | Phe |
| | 0006400 | | | | | | D-Trp | | | |
| 309 | RB- | DOTA-Lu | GABA | | NMe-Lys | Tyr | (S-βMe) | Cha4N | Thr | Phe |
| | 0006403 | | | | | | D-Trp | | | |
| 310 | RB- | DOTA-Lu | Apa | | NMe-Lys | Tyr | (S-βMe) | Cha4N | Thr | Phe |
| | 0006406 | | | | | | D-Trp | | | |
| 311 | RB- | ((R)- | | | NMe-Lys | Tyr | (S-βMe) | PipzaA | Thr | Phe |
| | 0006414 | NODAGA- | | | | | D-Trp | | | |
| | | Ga) | | | | | | | | |
| 312 | RB- | (R/S)- | | | NMe-Lys | Tyr | (S-βMe) | PipzaA | Thr | Phe |
| | 0006417 | NODAGA- | | | | | D-Trp | | | |
| | | Ga | | | | | | | | |
| 313 | RB- | DOTA-Lu | TXA | | NMe-Lys | Tyr | (S-βMe) | PipzaA | Thr | Phe |
| | 0006420 | | | | | | D-Trp | | | |
| 314 | RB- | DOTA-Lu | | | NMe-hLys | Tyr | (S-βMe) | Cha4N | Thr | Phe |
| | 0006449 | | | | | | D-Trp | | | |
| 315 | RB- | DOTA-Lu | 4PipCA | | NMe-Lys | Tyr | (S-βMe) | Cha4N | Thr | Phe |
| | 0006452 | | | | | | D-Trp | | | |
| 316 | RB- | DOTA-Lu | 3AzeCA | | NMe-Lys | Tyr | (S-βMe) | Cha4N | Thr | Phe |
| | 0006455 | | | | | | D-Trp | | | |
| 317 | RB- | DOTA-Lu | 4Pipaa | | NMe-Lys | Tyr | (S-βMe) | Cha4N | Thr | Phe |
| | 0006458 | | | | | | D-Trp | | | |
| 318 | RB- | DOTA-Lu | 3Azeaa | | NMe-Lys | Tyr | (S-βMe) | Cha4N | Thr | Phe |
| | 0006461 | | | | | | D-Trp | | | |
| 319 | RB- | DOTA-Lu | TXA | | NMe-Lys | Tyr | (S-βMe) | Cha4N | Thr | Phe |
| | 0006464 | | | | | | D-Trp | | | |
| 320 | RB- | ((R)- | TXA | | NMe-Lys | Tyr | (S-βMe) | Cha4N | Thr | Phe |
| | 0006470 | NODAGA- | | | | | D-Trp | | | |
| | | Ga) | | | | | | | | |
| 321 | RB- | ((R)- | | | NMe-Lys | Tyr | (S-βMe) | Cha4N | Thr | Phe |
| | 0006476 | NODAGA- | | | | | D-Trp | | | |
| | | Ga) | | | | | | | | |
| 322 | RB- | NOTA-Ga | | | NMe-Lys | Tyr | (S-βMe) | PipzaA | Thr | Phe |
| | 0006488 | | | | | | D-Trp | | | |
| 323 | RB- | DOTA-Lu | | | NMe-Lys | Tyr | 2-aza-(D- | Cha4N | Thr | Phe |
| | 0006553 | | | | | | Trp) | | | |
| 324 | RB- | DOTA-Lu | 2.2.2.Octadma | | NMe-Lys | Tyr | (S-βMe) | Cha4N | Thr | Phe |
| | 0006558 | | | | | | D-Trp | | | |
| 325 | RB- | DOTA-Lu | | | NMe- | Tyr | (S-βMe) | Cha4N | Thr | Phe |
| | 0006579 | | | | Lys(Me) | | D-Trp | | | |
| 326 | RB- | DOTA-Lu | triazole | | NMe-Lys | Tyr | (S-βMe) | Cha4N | Thr | Phe |
| | 0006591 | | | | | | D-Trp | | | |

Wherein in Table 2, “Ac” is Acetyl; “2.2.2.Octadma” is 4-(aminomethyl)bicyclo[2.2.2]octane-1-carboxylic acid; “3Azeaa” is 2-(azetidin-3-yl)acetic acid; “3AzeCA” is azetidine-3-carboxylic acid; “4Pipaa” is 2-(piperidin-4-yl)acetic acid; “4PipCA” is piperidine-4-carboxylic acid; “Aeea” is 2-(2-(2-(2-aminoethoxy)ethoxy)ethoxy)acetic acid; “Ahx” is 6-aminohexanoic acid; “Aoc” is 8-aminooctanoic acid; “Apa” is 5-aminopentanoic acid; “CDAE” is carbonyldiaminoethyl; “Dah” is hexane-1,6-diamine; “Daop” is 2,2′-oxybis(ethan-1-amine); “Dap” is pentane-1,5-diamine; “en” is ethane-1,2-diamine; and “TXA” is 4-(aminomethyl)cyclohexane-1-carboxylic acid.

Exemplary conjugates of the disclosure are further illustrated in Table 3 below.

TABLE 3

| Conjugate ID | |
|---|---|
| (SEQ ID NO) | Conjugate Structure |
| RB-0002669 (145) | 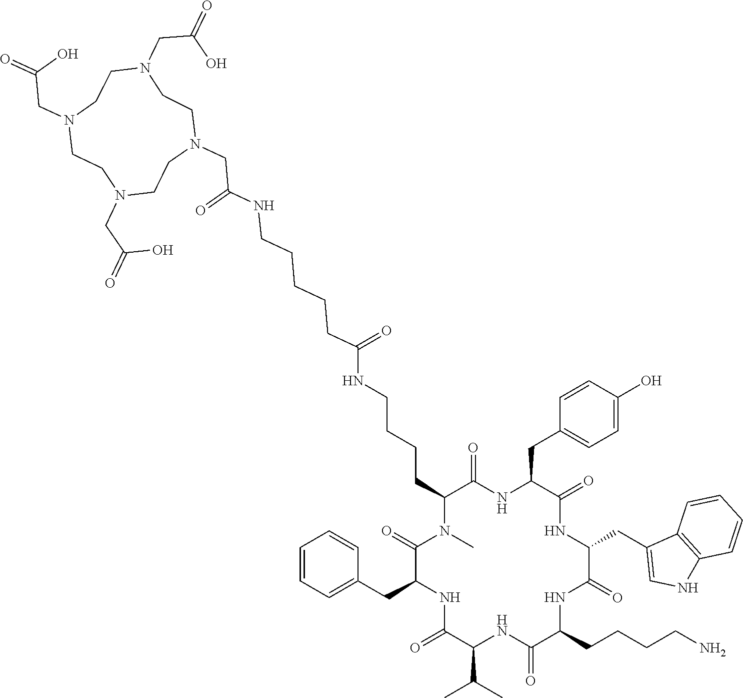 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCNC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0002671 (146) | 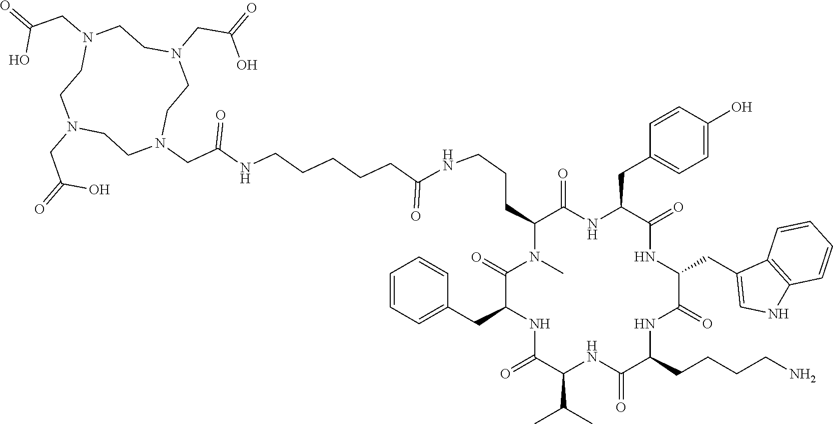 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCNC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0002673 (147) | 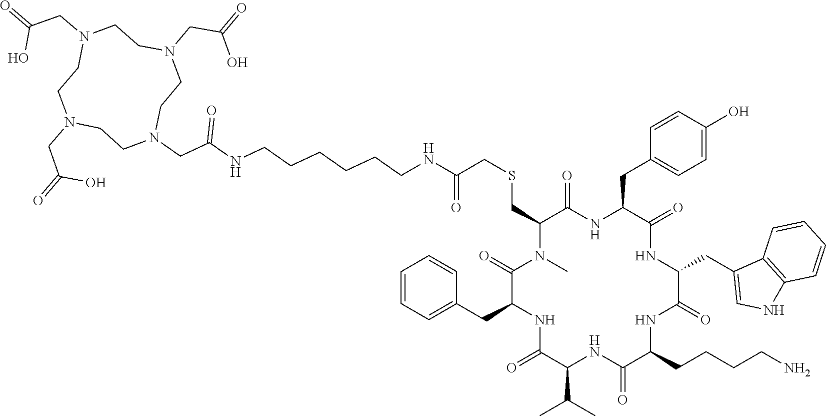 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0002675 (148) | 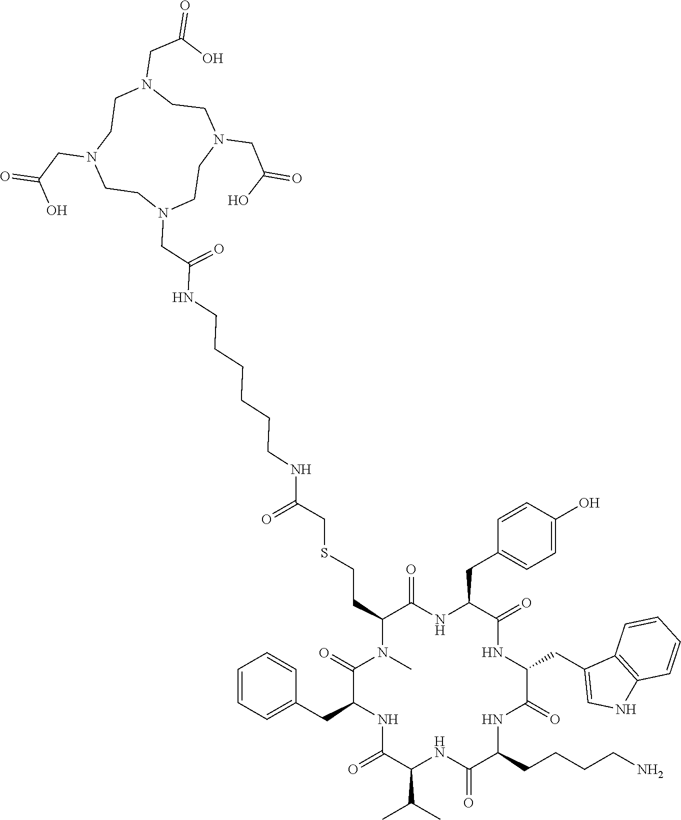 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0002677 (149) | 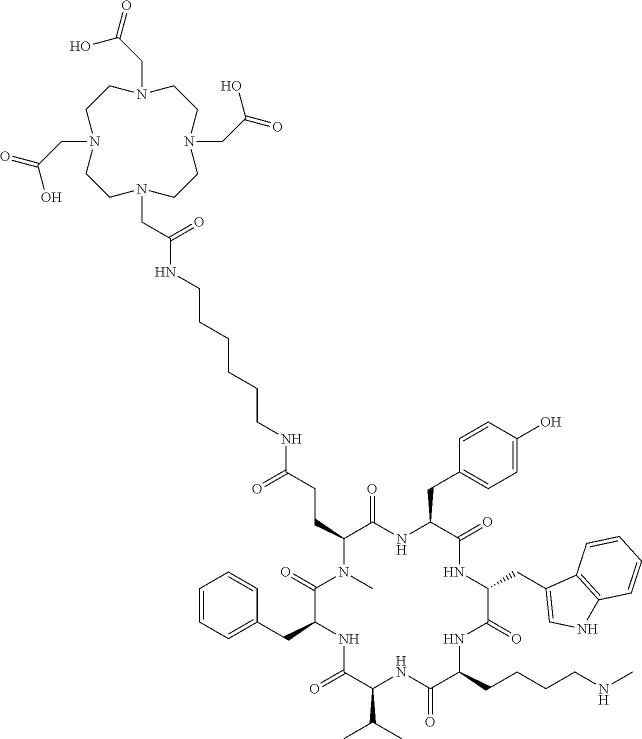 [SMILES: CNCCCC[C@@H]1NC(=O)[C@@H](Cc2c[nH]c3ccccc23)NC(=O)[C@H](Cc2ccc(O)cc2)NC(=O)[C@H](CCC(=O)NCCCCCCNC(=O)CN2CCN(CC(=O)O)CCN(CC(=O)O)CCN(CC(=O)O)CC2)N(C)C(=O)[C@H](Cc2ccccc2)NC(=O)[C@H](C(C)C)NC1=O] |
| RB-0002679 (150) | 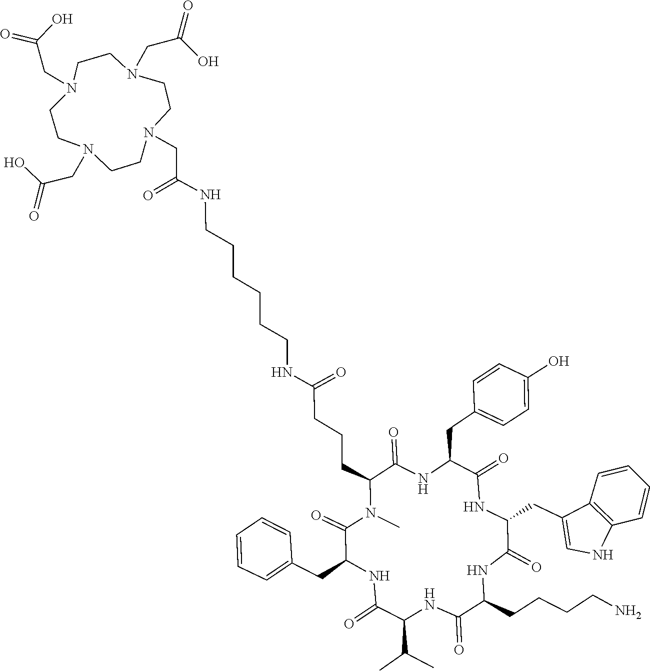 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0002980 (151) | 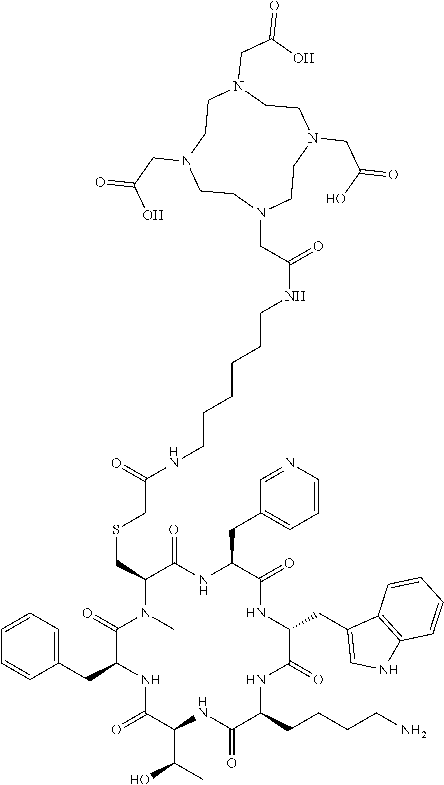 [SMILES: C[C@@H](O)[C@@H]1NC(=O)[C@H](CCCCN)NC(=O)[C@@H](Cc2c[nH]c3ccccc23)NC(=O)[C@H](Cc2cccnc2)NC(=O)[C@H](CSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0002999 (153) | 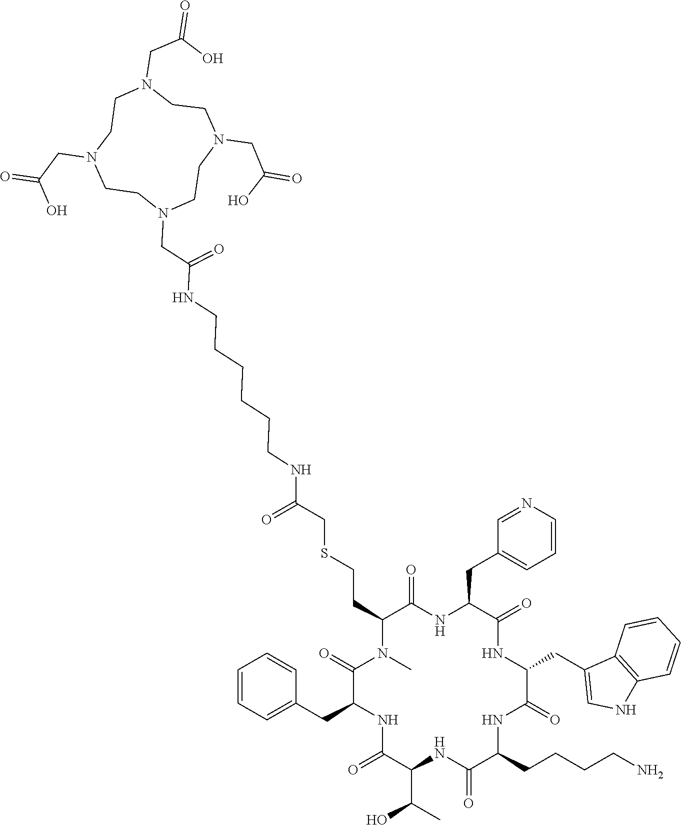 [SMILES: C[C@@H](O)[C@@H]1NC(=O)[C@H](CCCCN)NC(=O)[C@@H](Cc2c[nH]c3ccccc23)NC(=O)[C@H](Cc2cccnc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003001 (154) | 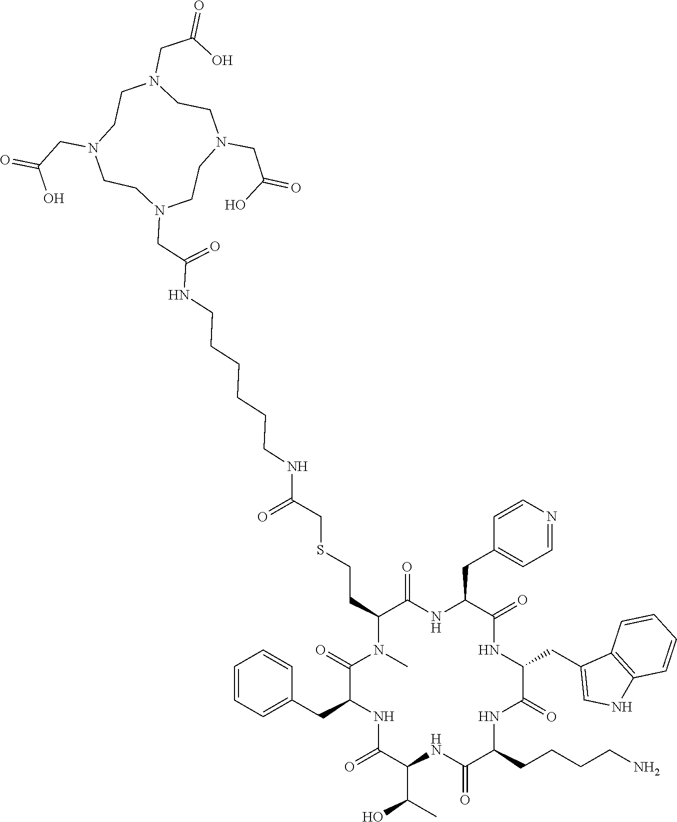 [SMILES: C[C@@H](O)[C@@H]1NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003054 (155) | 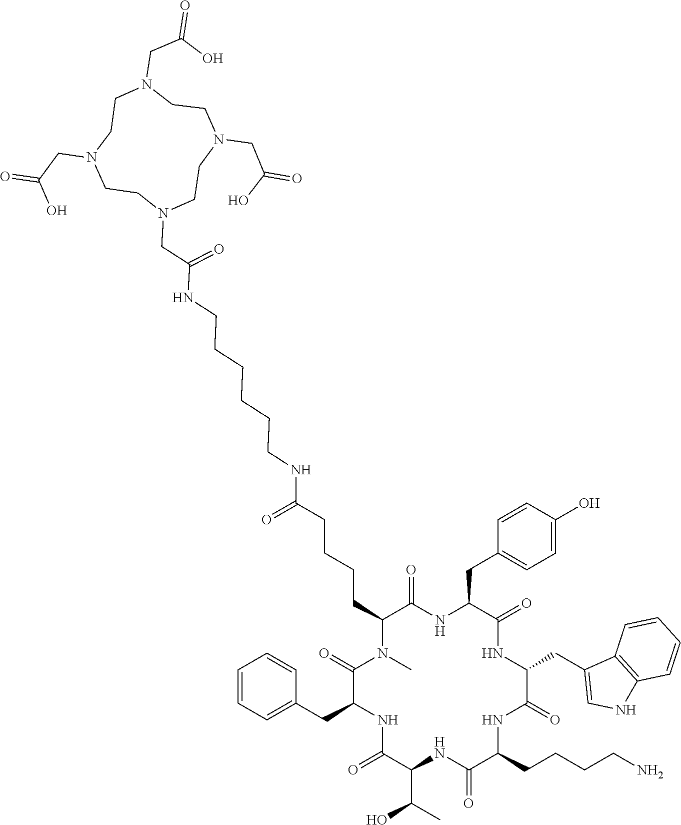 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003056 (156) | 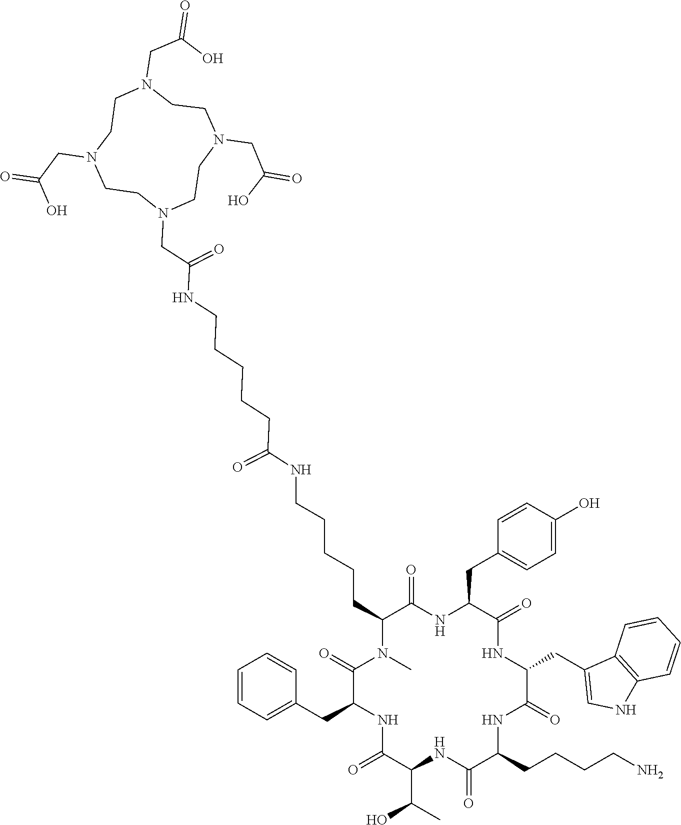 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCCNC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003111 (159) | 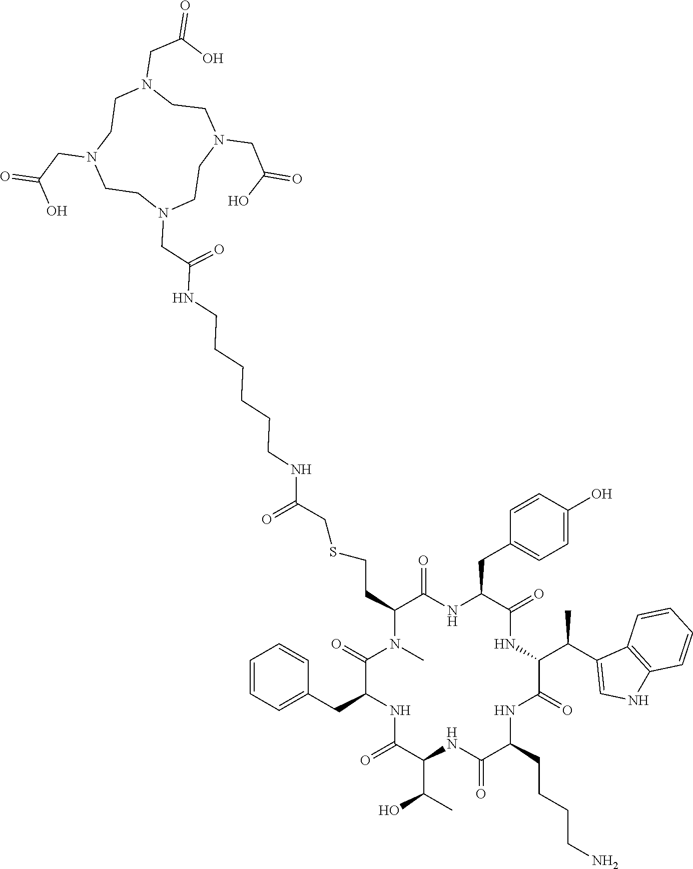 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003112 (160) | 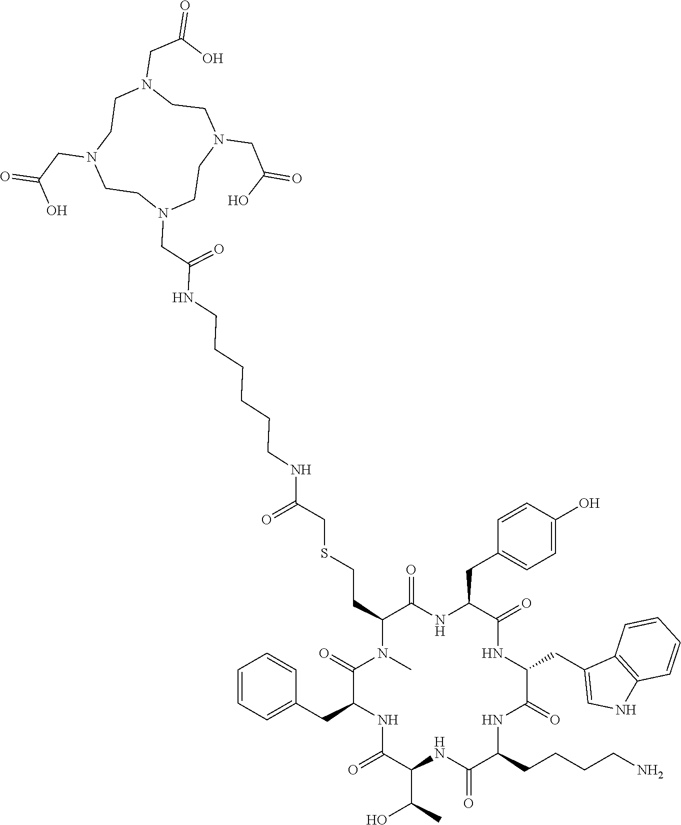 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003113 (158) | 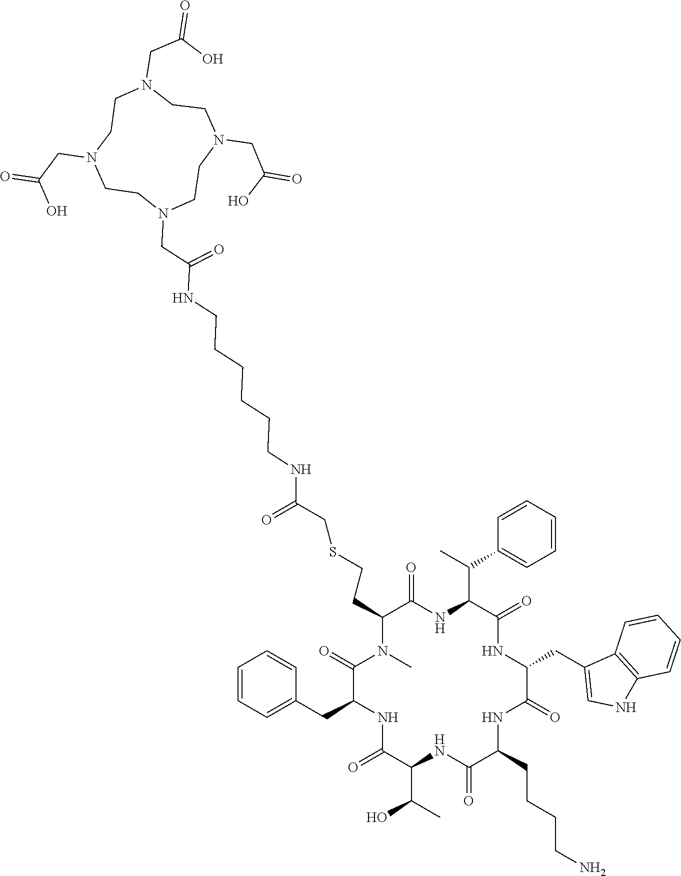 |
| RB-0003114 (157) | 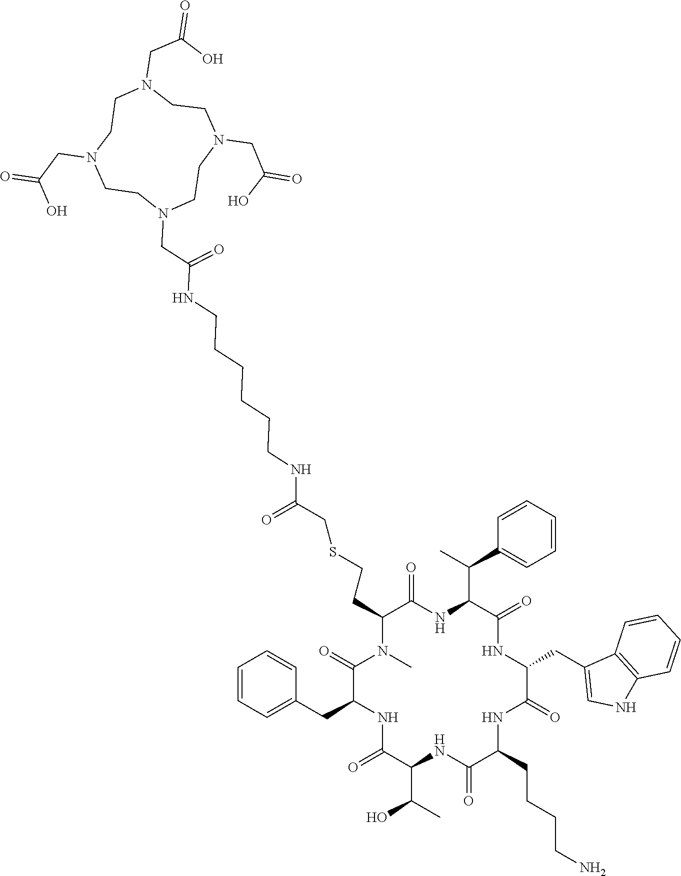 [SMILES: C[C@@H](O)[C@@H]1NC(=O)[C@H](CCCCN)NC(=O)[C@@H](Cc2c[nH]c3ccccc23)NC(=O)[C@H]([C@@H](C)c2ccccc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003138 (161) | 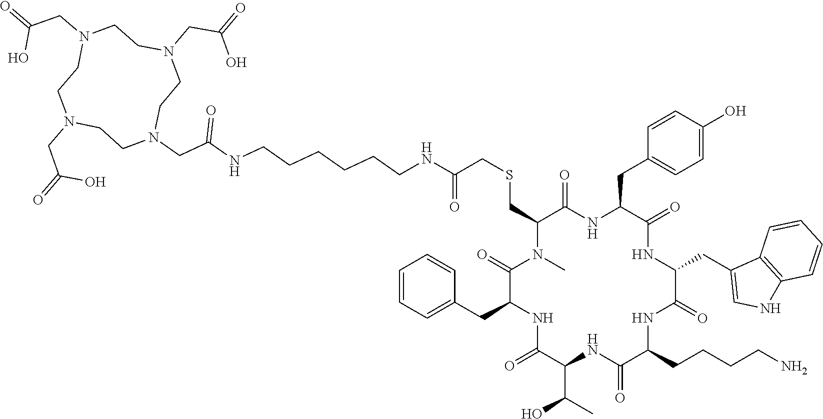 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003245 (162) | 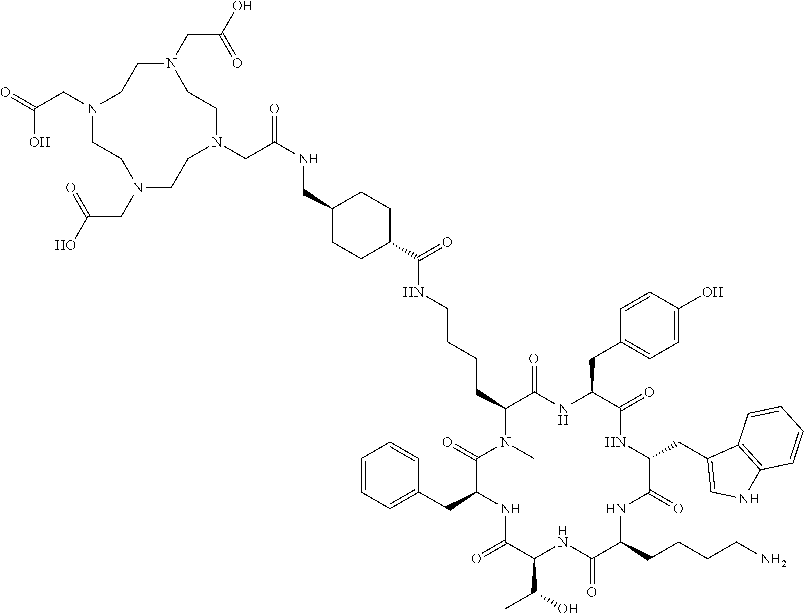 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCNC(=O)[C@H]2CC[C@H](CNC(=O)CN3CCN(CC(=O)O)CCN(CC(=O)O)CCN(CC(=O)O)CC3)CC2)N(C)C(=O)[C@H](Cc2ccccc2)NC1=O] |
| RB-0003270 (164) | 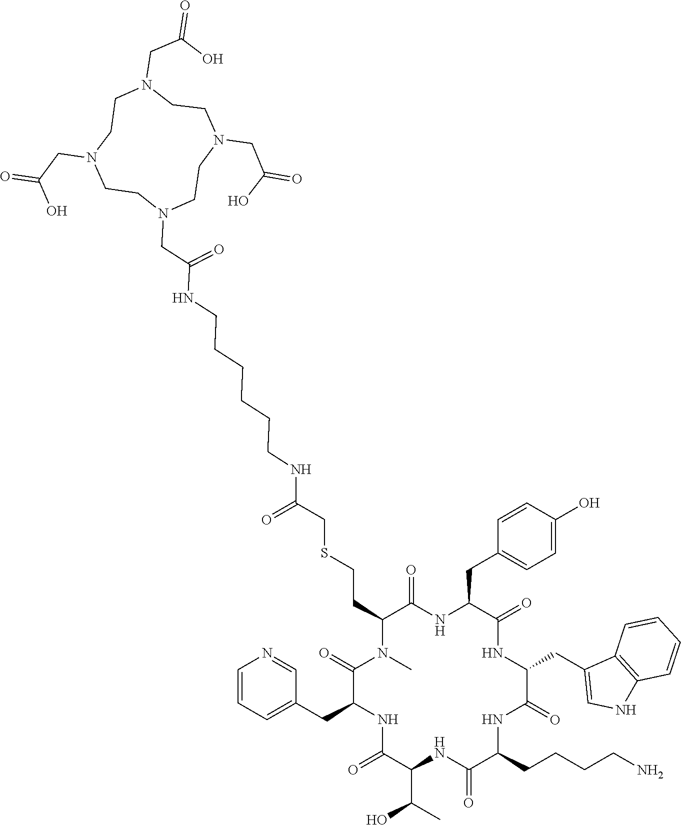 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2cccnc2)NC1=O] |
| RB-0003271 (165) | 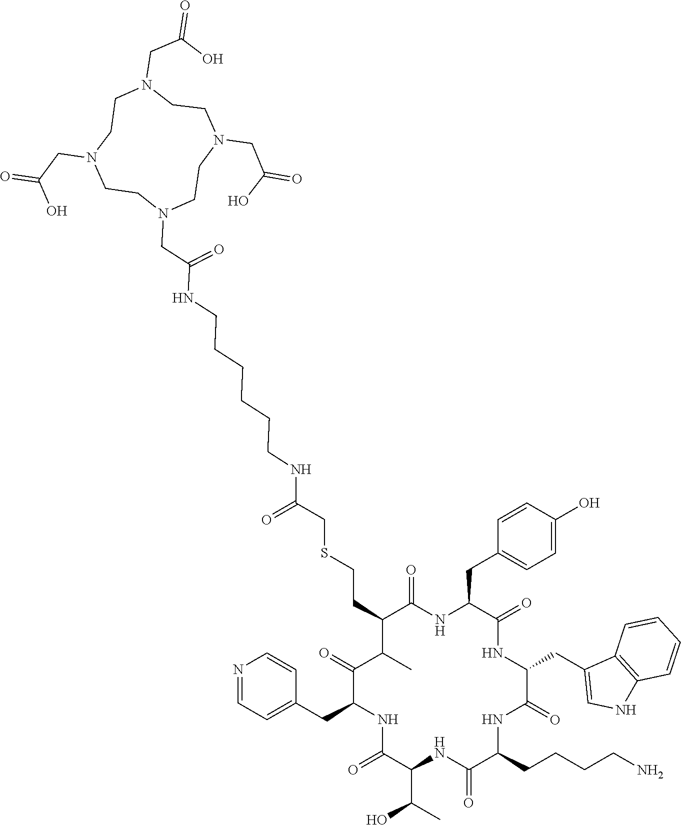 [SMILES: CC1C(=O)[C@H](Cc2ccncc2)NC(=O)[C@H]([C@@H](C)O)NC(=O)[C@H](CCCCN)NC(=O)[C@@H](Cc2c[nH]c3ccccc23)NC(=O)[C@H](Cc2ccc(O)cc2)NC(=O)[C@@H]1CCSCC(=O)NCCCCCCNC(=O)CN1CCN(CC(=O)O)CCN(CC(=O)O)CCN(CC(=O)O)CC1] |
| RB-0003272 (166) | 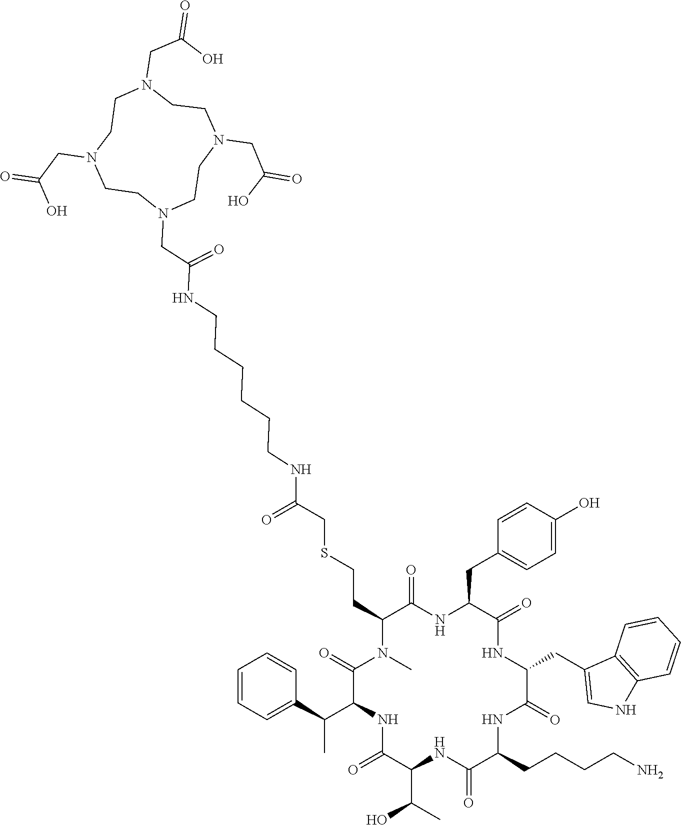 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H]([C@@H](C)c2ccccc2)NC1=O] |
| RB-0003273 (167) | 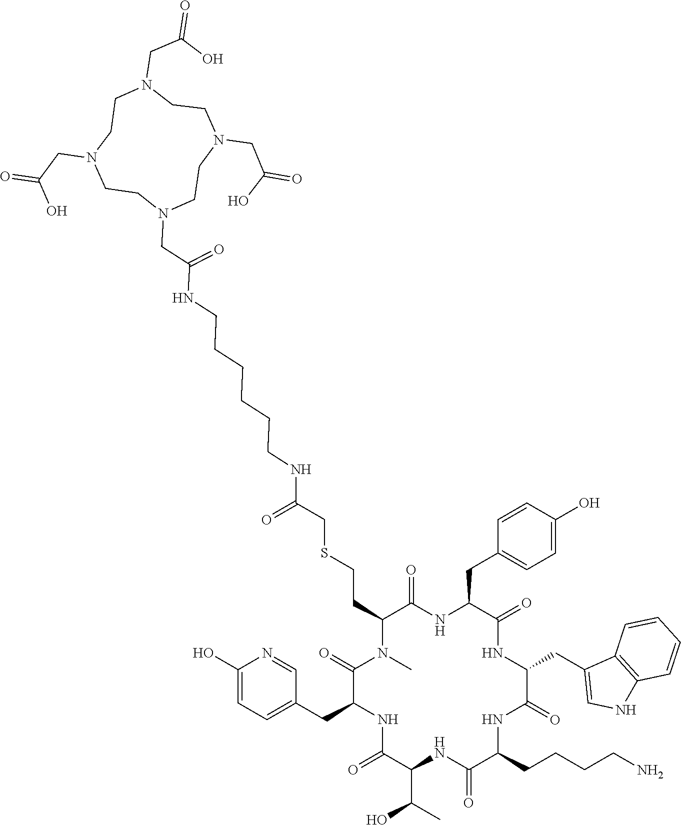 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccc(O)nc2)NC1=O] |
| RB-0003274 (168) | 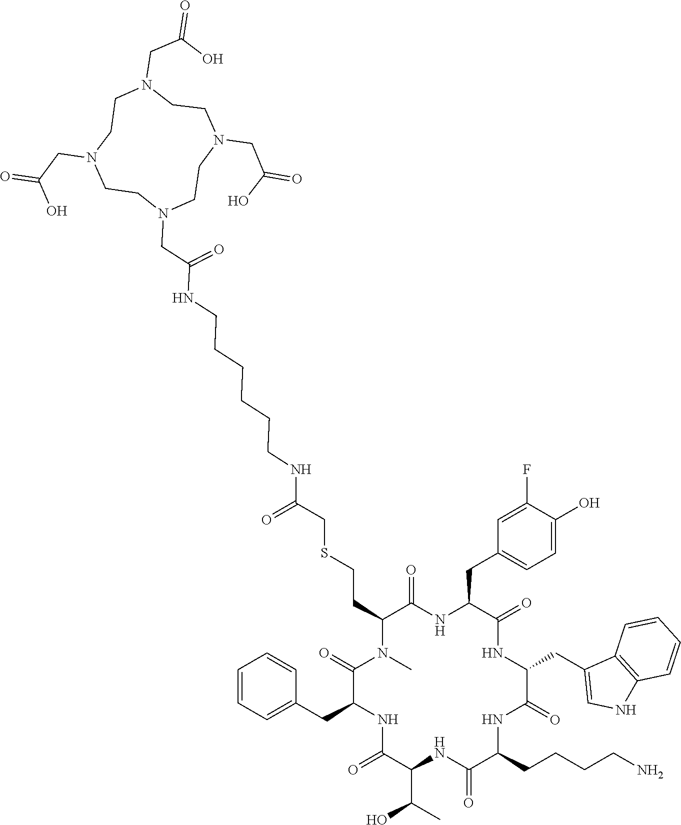 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)c(F)c2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003275 (169) | 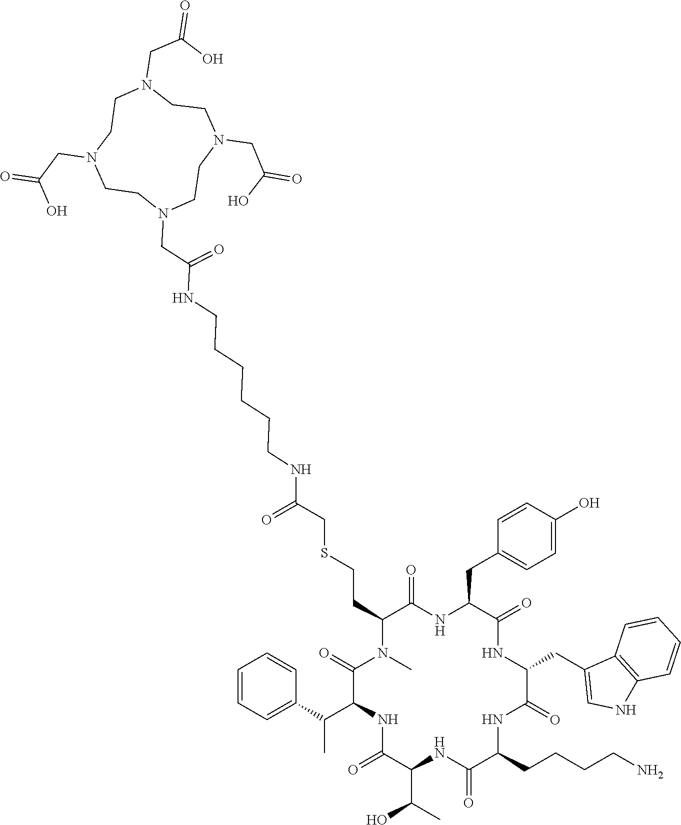 |
| RB-0003291 (170) | 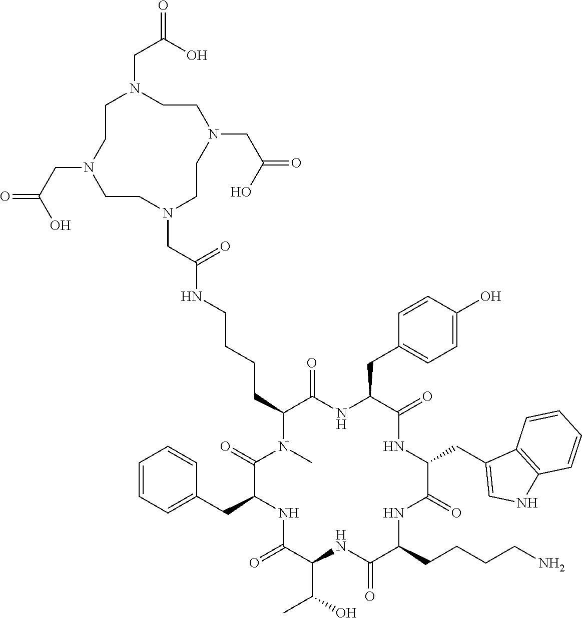 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003293 (171) | 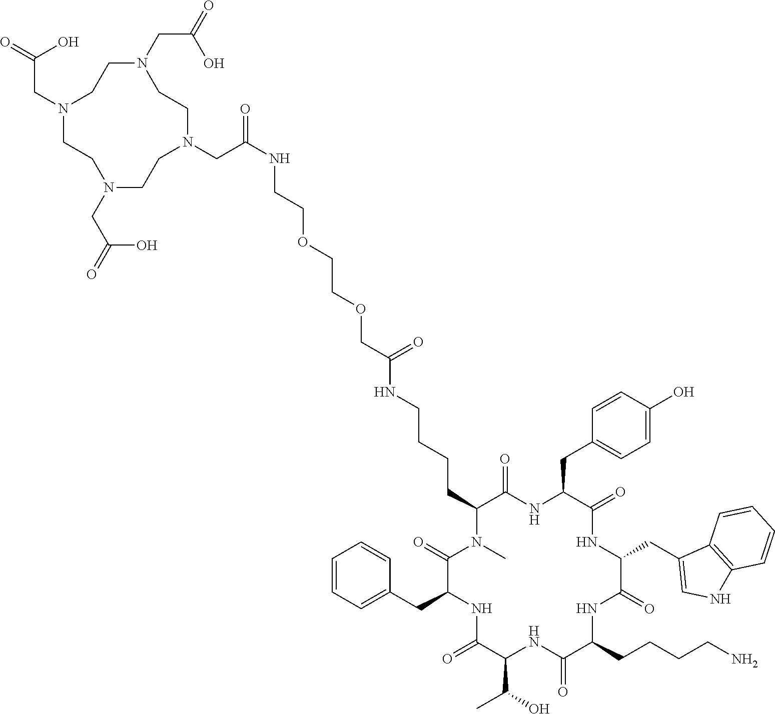 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCNC(=O)COCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0003295 (171) | 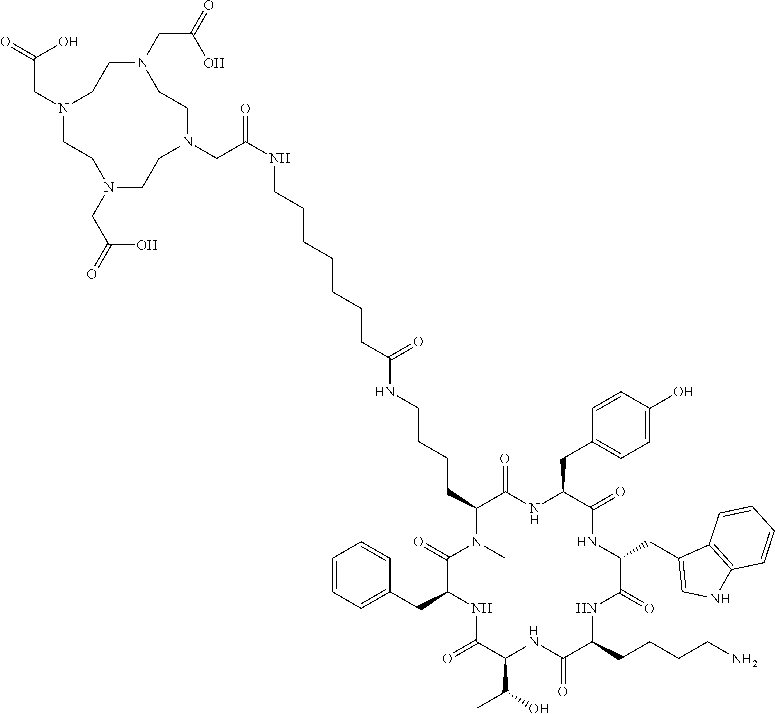 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCNC(=O)CCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003297 (173) | 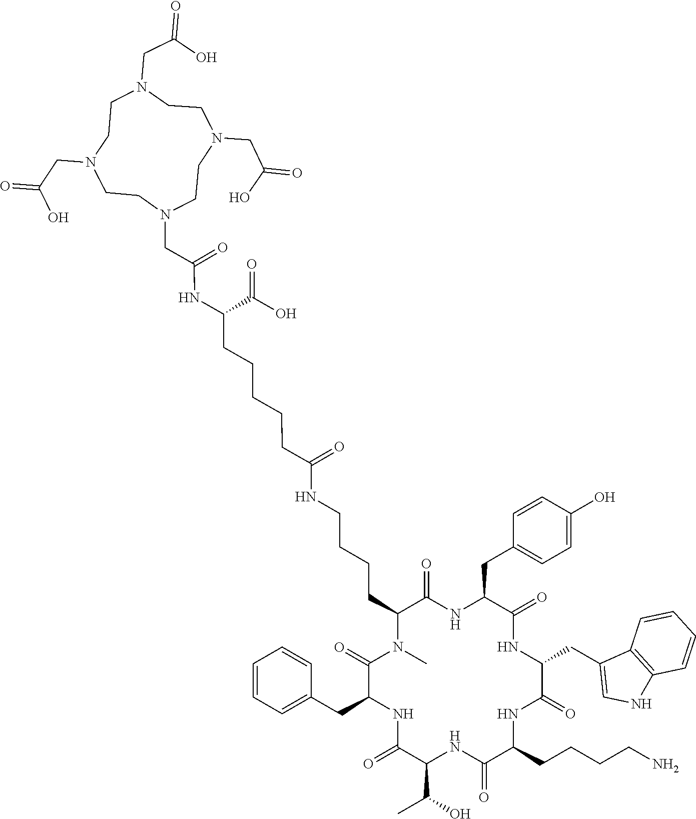 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCNC(=O)CCCCC[C@H](NC(=O)CN2CCN(CC(=O)O)CCN(CC(=O)O)CCN(CC(=O)O)CC2)C(=O)O)N(C)C(=O)[C@H](Cc2ccccc2)NC1=O] |
| RB-0003301 (174) | 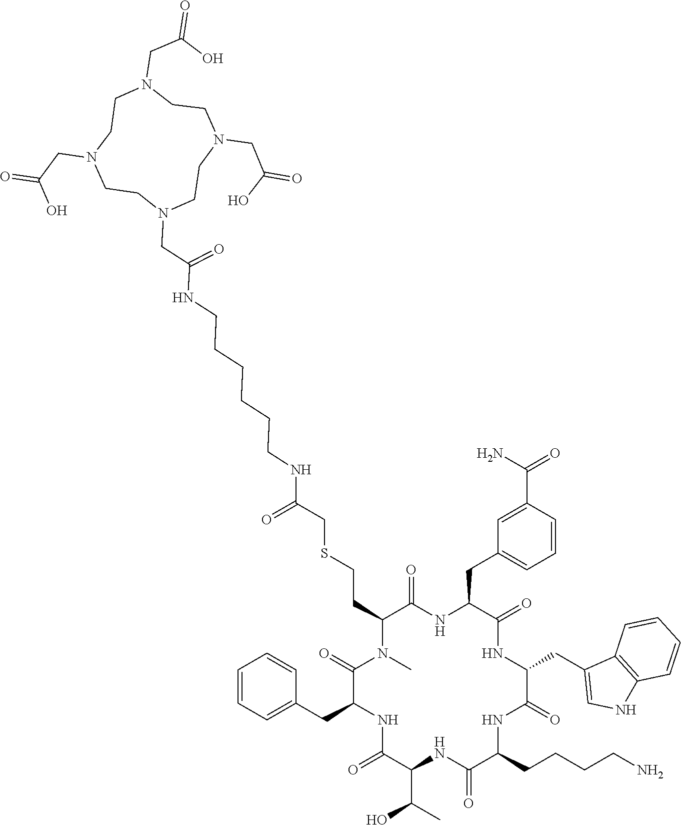 [SMILES: C[C@@H](O)[C@@H]1NC(=O)[C@H](CCCCN)NC(=O)[C@@H](Cc2c[nH]c3ccccc23)NC(=O)[C@H](Cc2cccc(C(N)=O)c2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003303 (175) | 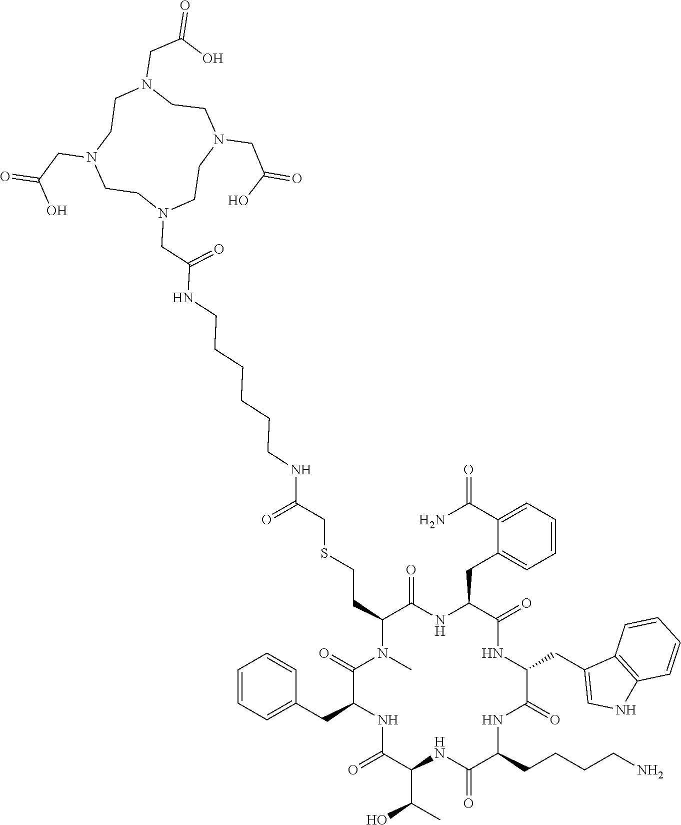 [SMILES: C[C@@H](O)[C@@H]1NC(=O)[C@H](CCCCN)NC(=O)[C@@H](Cc2c[nH]c3ccccc23)NC(=O)[C@H](Cc2ccccc2C(N)=O)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003305 (176) | 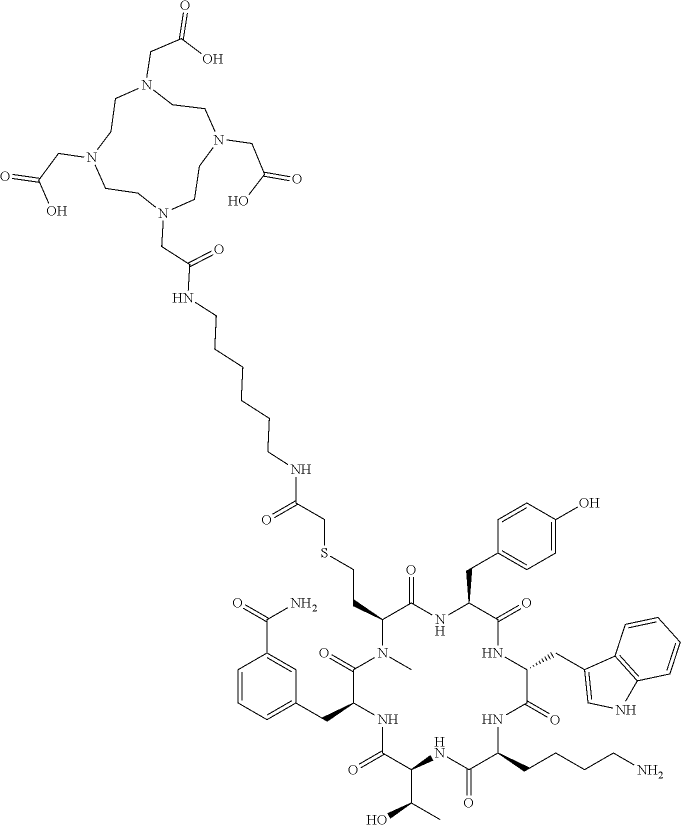 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2cccc(C(N)=O)c2)NC1=O] |
| RB-0003307 (194) | 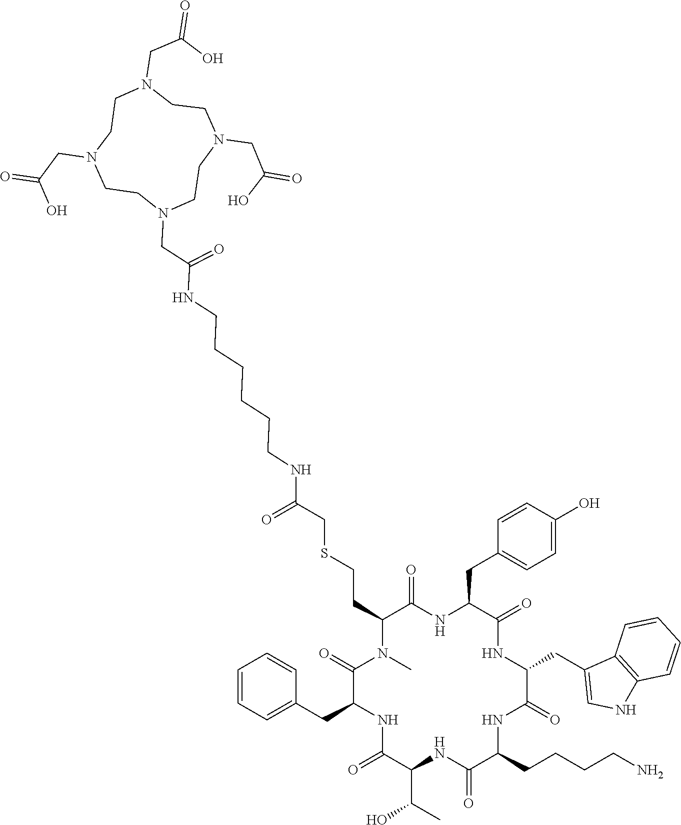 [SMILES: C[C@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003308 (177) | 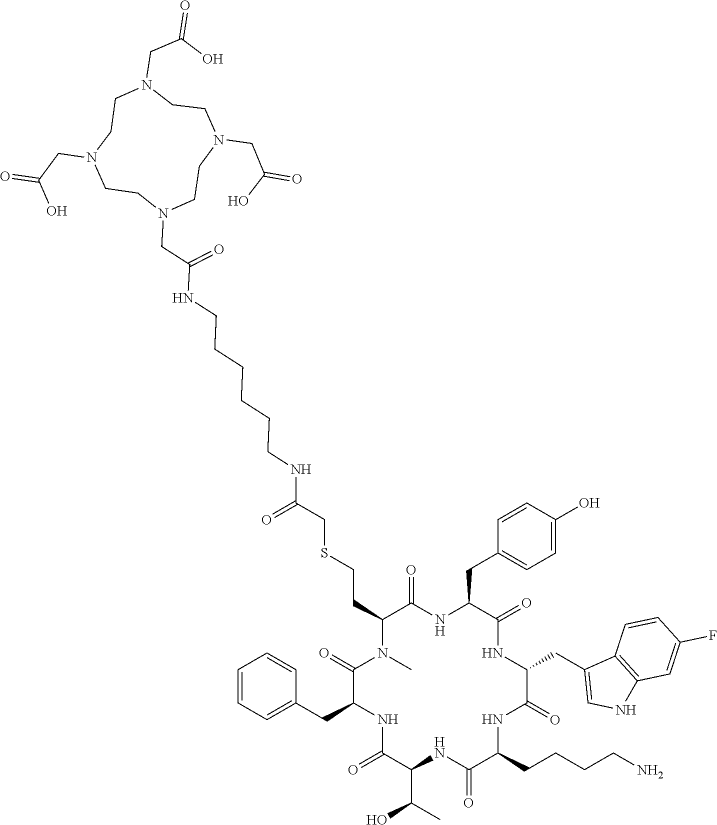 [SMILES: C[C@@H](O)[C@@H]1NC(=O)[C@H](CCCCN)NC(=O)[C@@H](Cc2c[nH]c3cc(F)ccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003337 (181) | 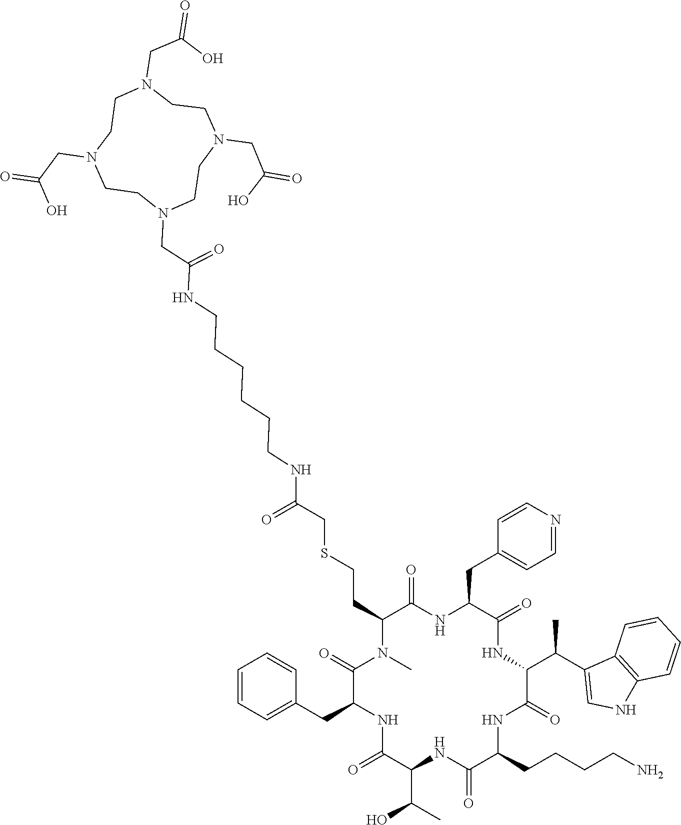 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003338 (182) | 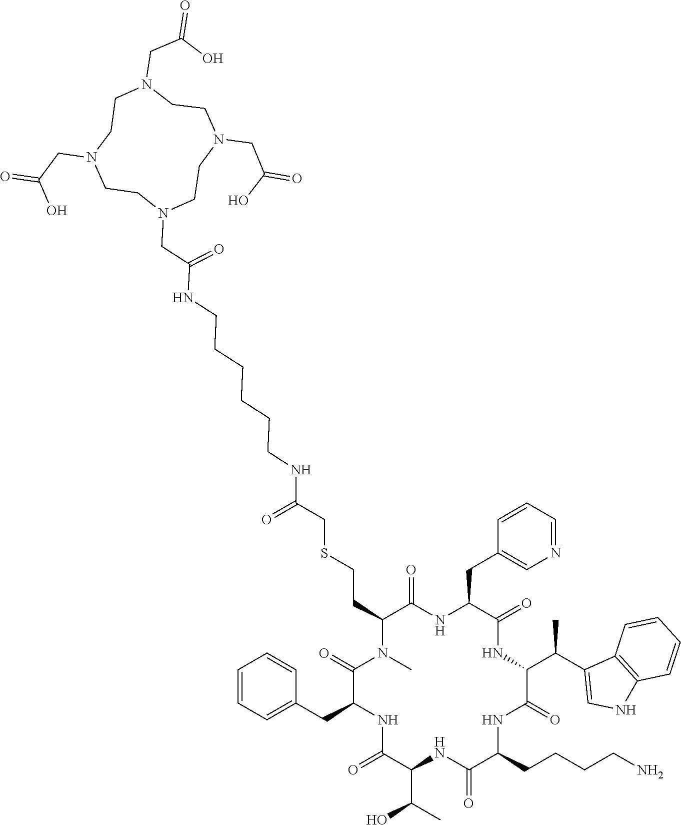 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2cccnc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003339 (183) | 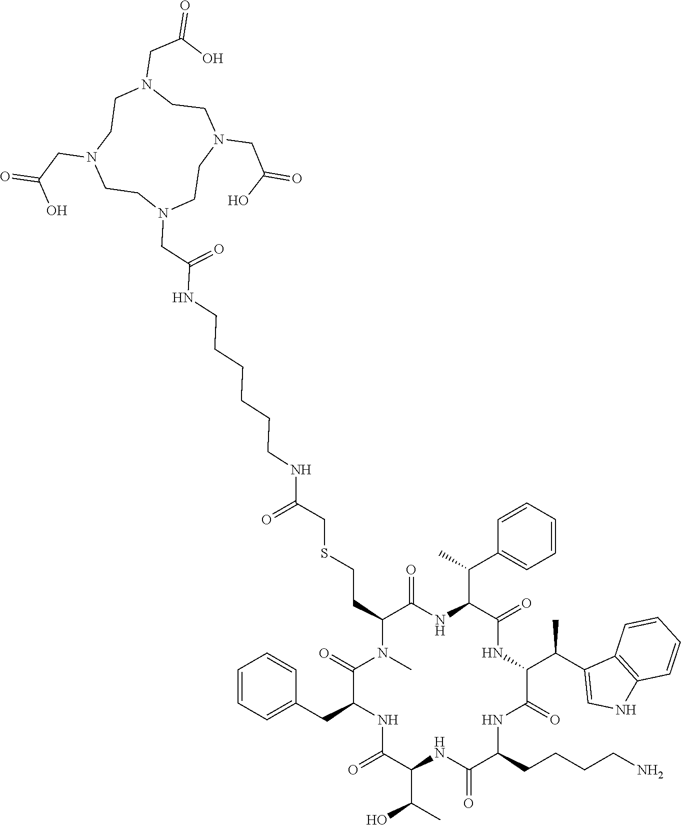 [SMILES: C[C@H](c1ccccc1)[C@@H]1NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC(=O)[C@H]([C@@H](C)O)NC(=O)[C@H](CCCCN)NC(=O)[C@@H]([C@@H](C)c2c[nH]c3ccccc23)NC1=O] |
| RB-0003356 (178) | 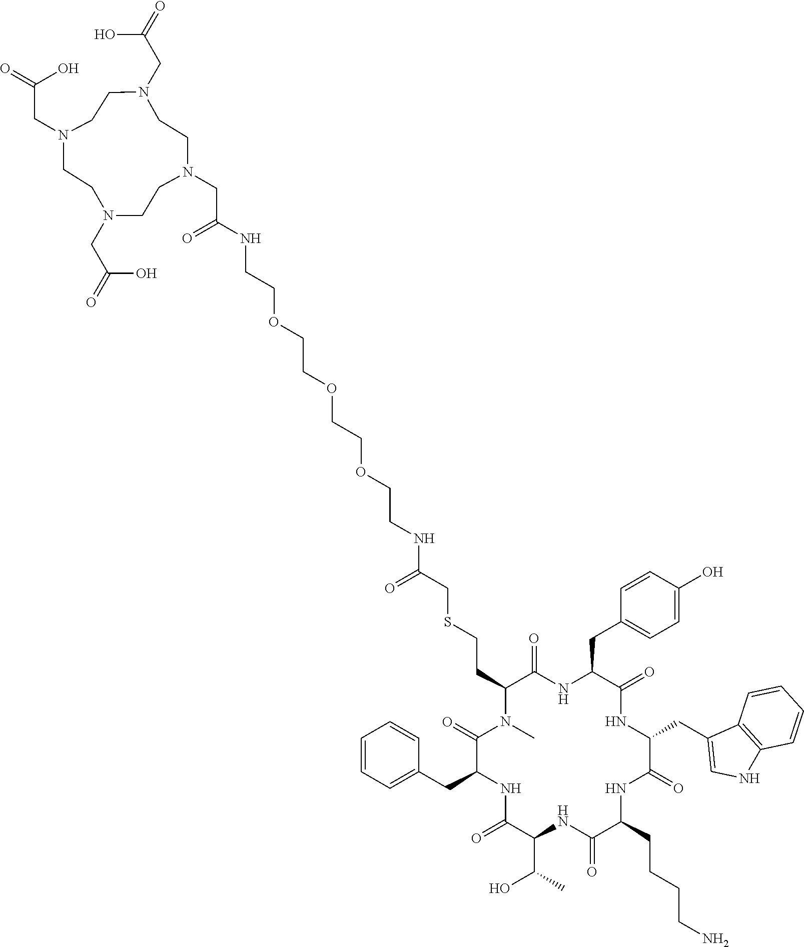 [SMILES: C[C@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0003407 (184) | 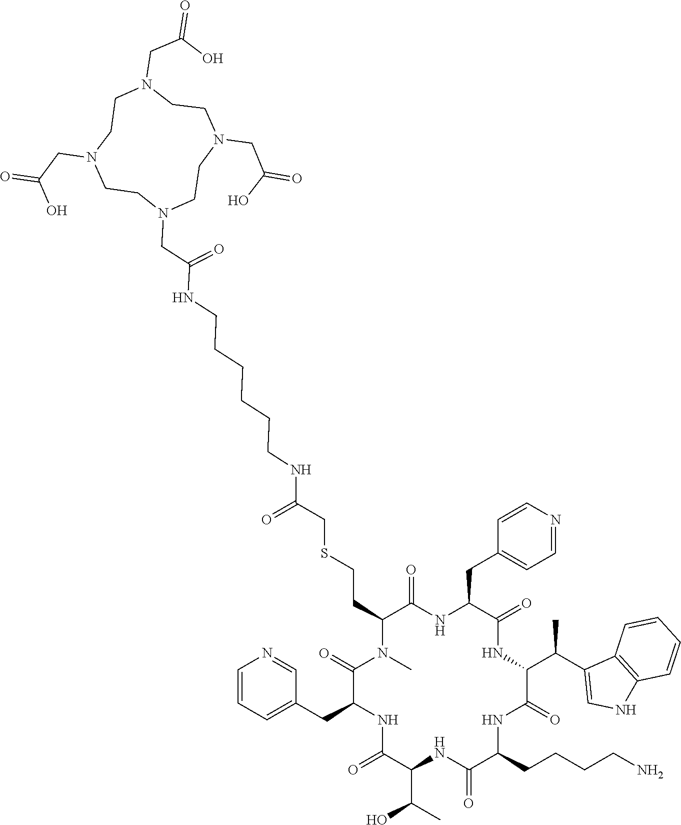 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2cccnc2)NC1=O] |
| RB-0003408 (185) | 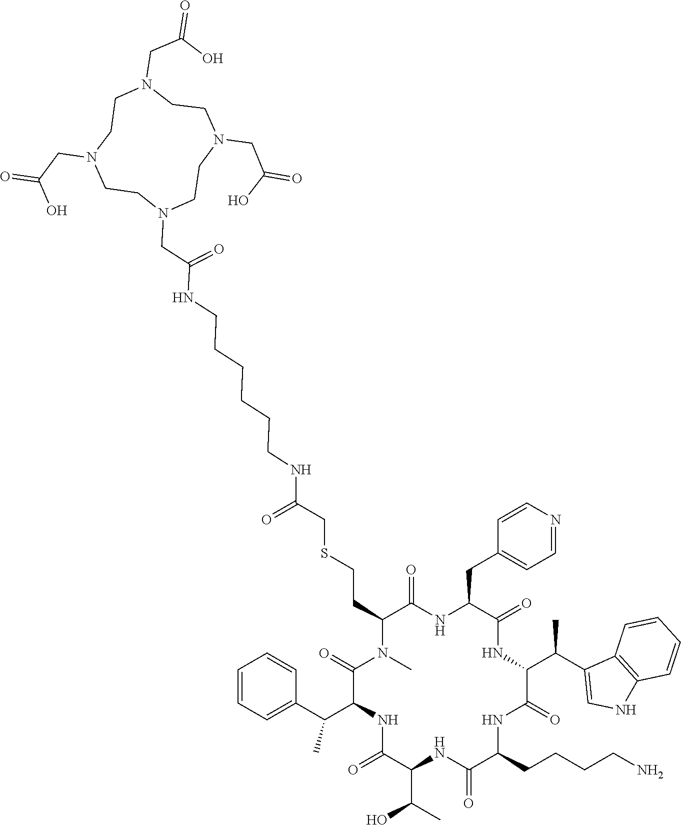 [SMILES: C[C@H](c1ccccc1)[C@@H]1NC(=O)[C@H]([C@@H](C)O)NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C1=O] |
| RB-0003409 (186) | 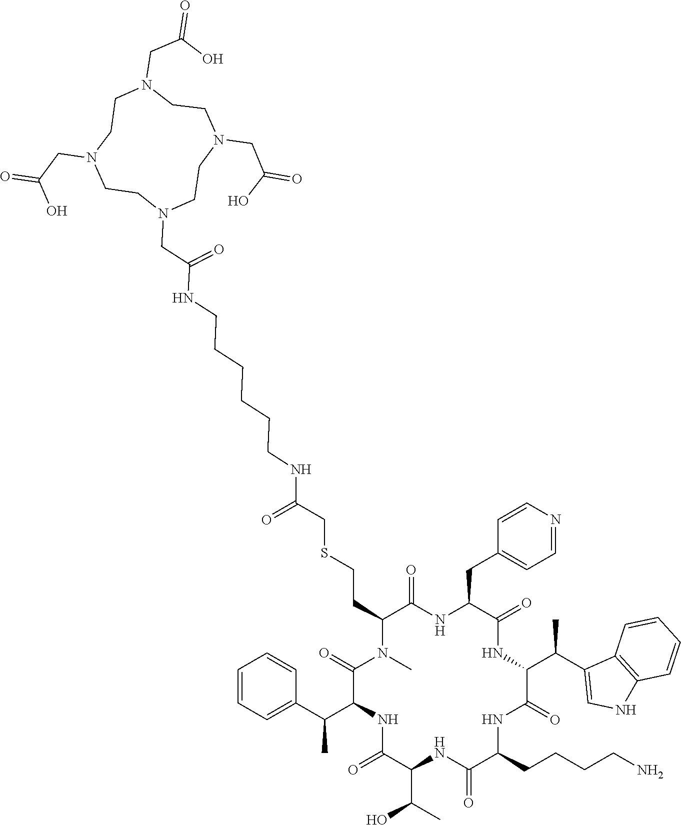 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H]([C@@H](C)c2ccccc2)NC1=O] |
| RB-0003412 (179) | 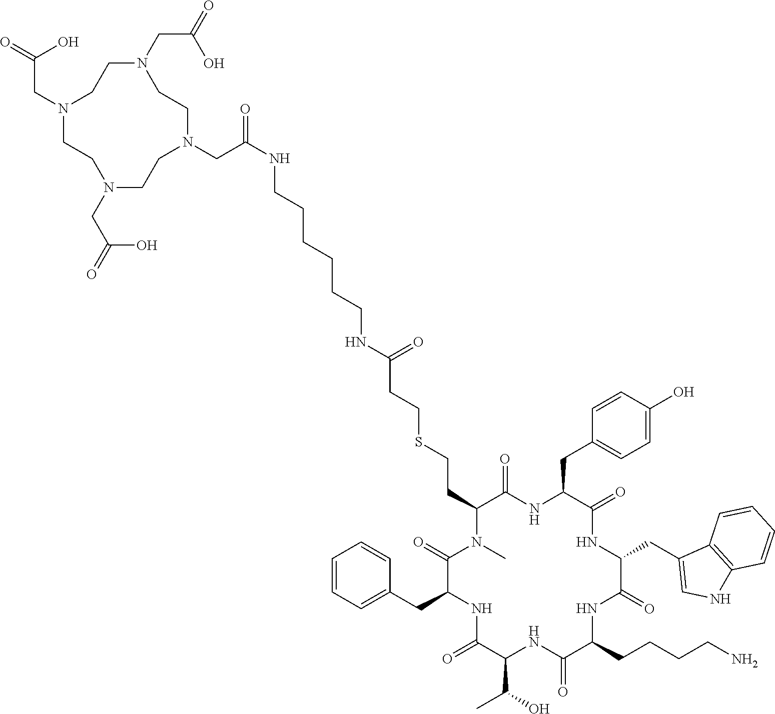 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003413 (180) | 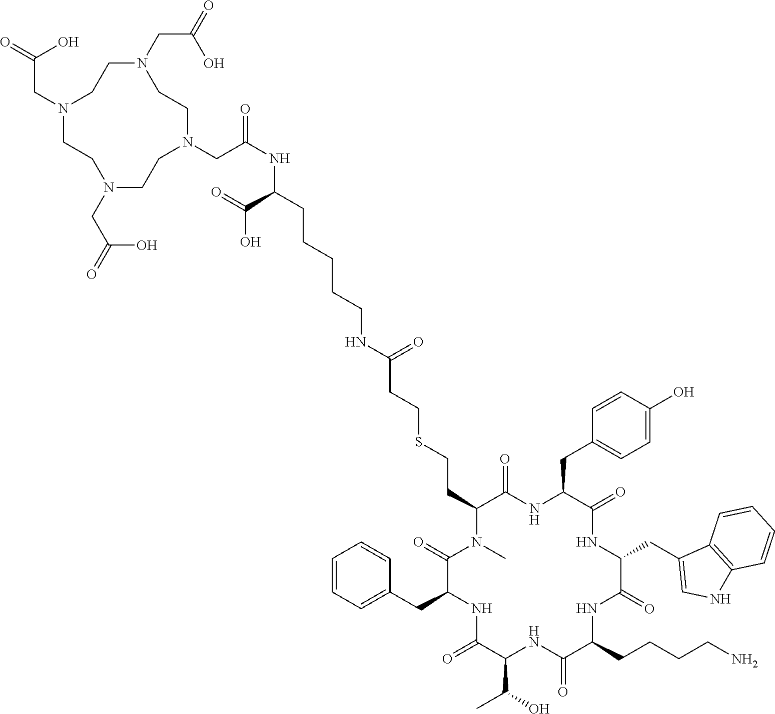 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCCC(=O)NCCCCC[C@H](NC(=O)CN2CCN(CC(=O)O)CCN(CC(=O)O)CCN(CC(=O)O)CC2)C(=O)O)N(C)C(=O)[C@H](Cc2ccccc2)NC1=O] |
| RB-0003442 (187) | 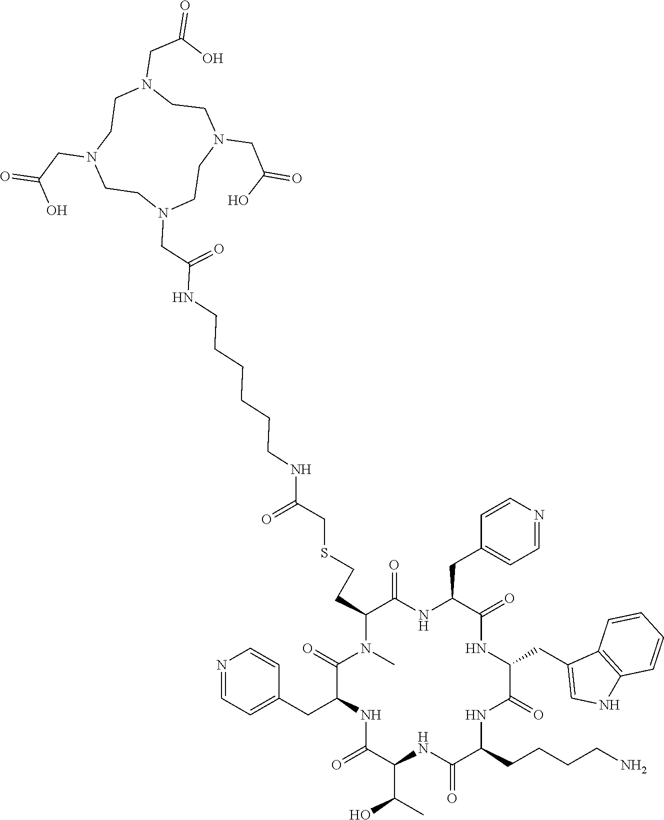 [SMILES: C[C@@H](O)[C@@H]1NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccncc2)NC1=O] |
| RB-0003444 (188) | 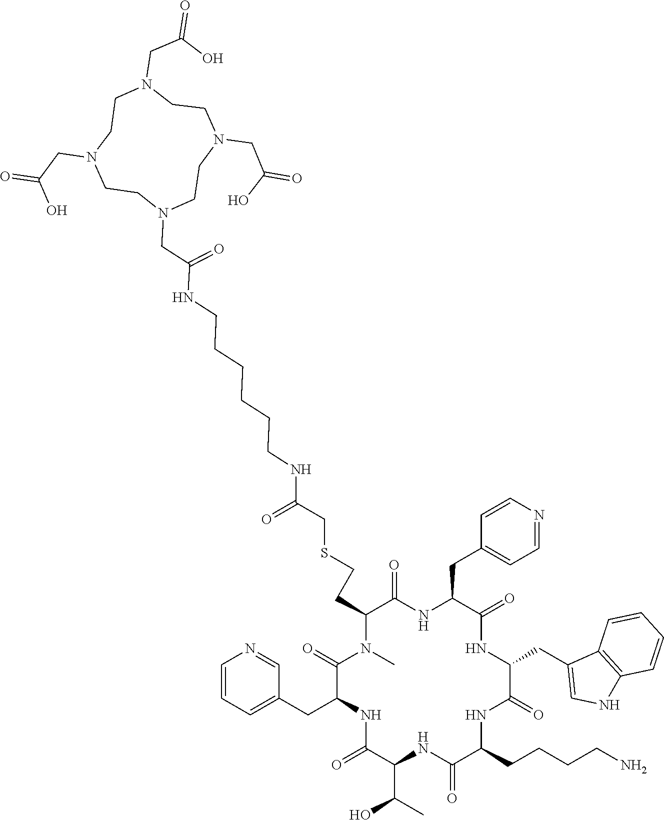 [SMILES: C[C@@H](O)[C@@H]1NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2cccnc2)NC1=O] |
| RB-0003446 (189) | 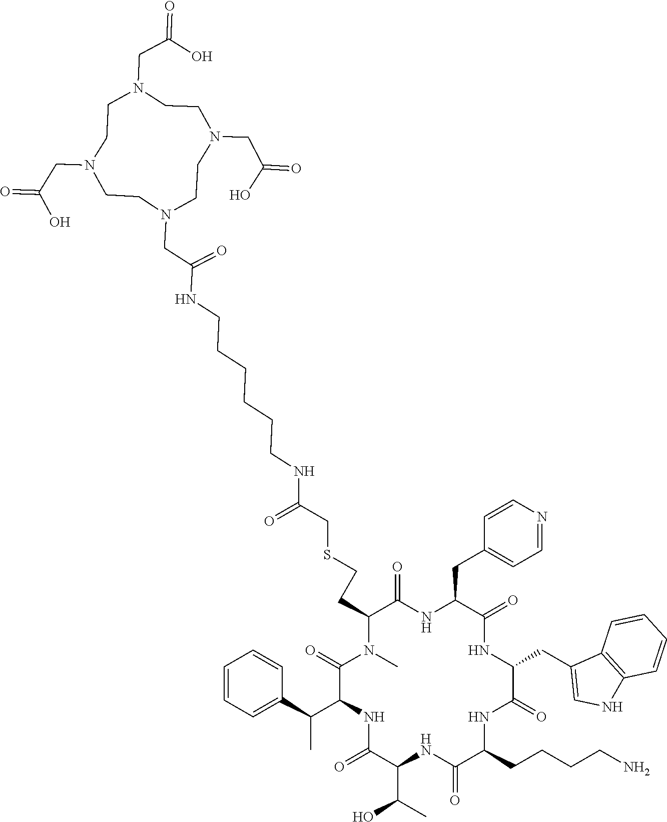 [SMILES: C[C@@H](O)[C@@H]1NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H]([C@@H](C)c2ccccc2)NC1=O] |
| RB-0003448 (190) | 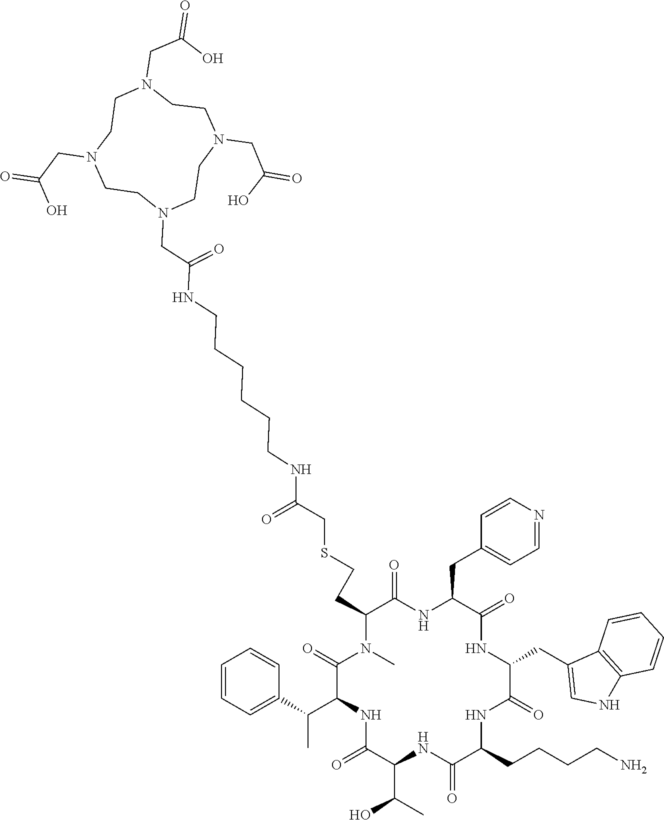 [SMILES: C[C@H](c1ccccc1)[C@@H]1NC(=O)[C@H]([C@@H](C)O)NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C1=O] |
| RB-0003450 (191) | 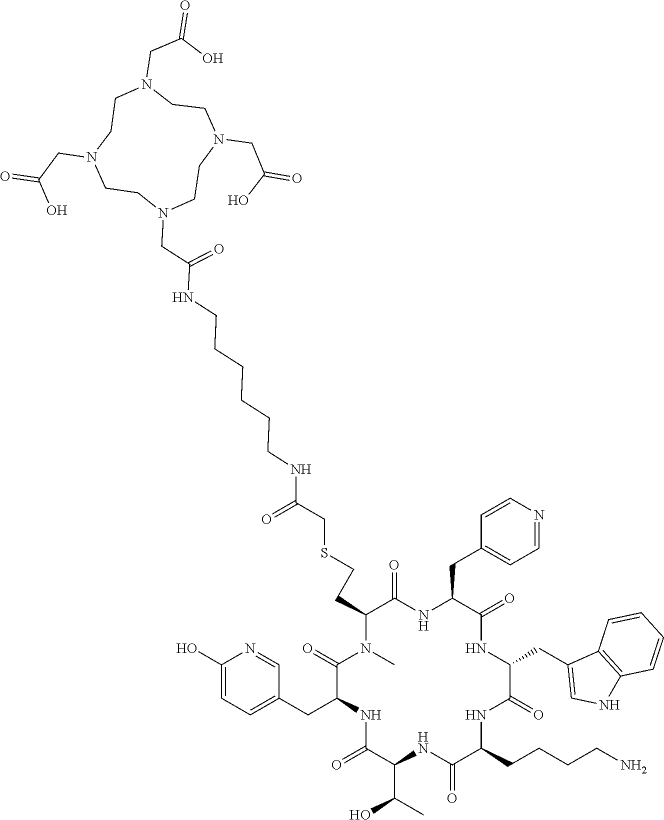 [SMILES: C[C@@H](O)[C@@H]1NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccc(O)nc2)NC1=O] |
| RB-0003452 (192) | 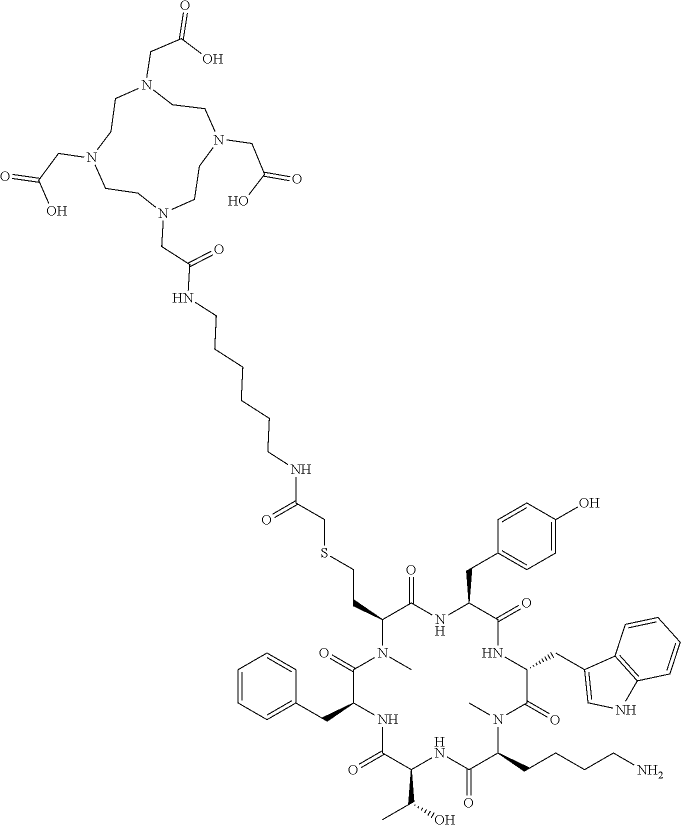 [SMILES: C[C@@H](O)[C@@H]1NC(=O)[C@H](CCCCN)N(C)C(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003454 (193) | 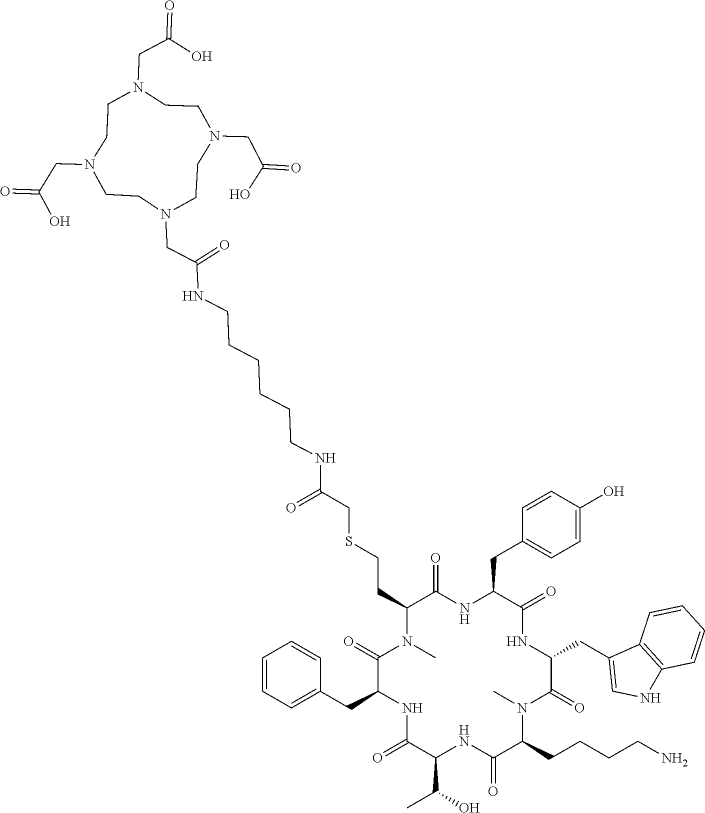 [SMILES: C[C@@H](O)[C@@H]1NC(=O)[C@H](CCCCN)N(C)C(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003481 (195) | 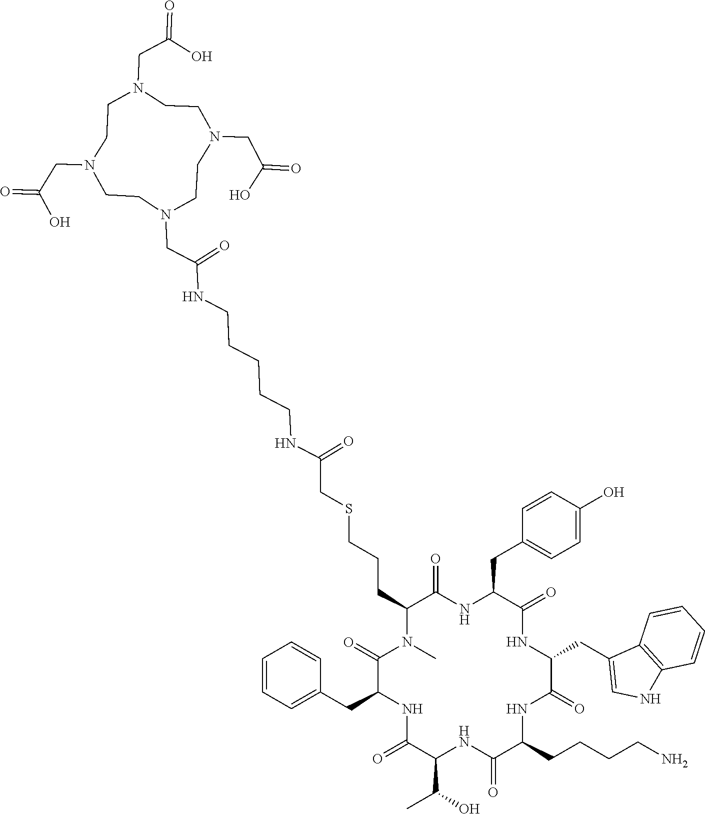 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCSCC(=O)NCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003483 (196) | 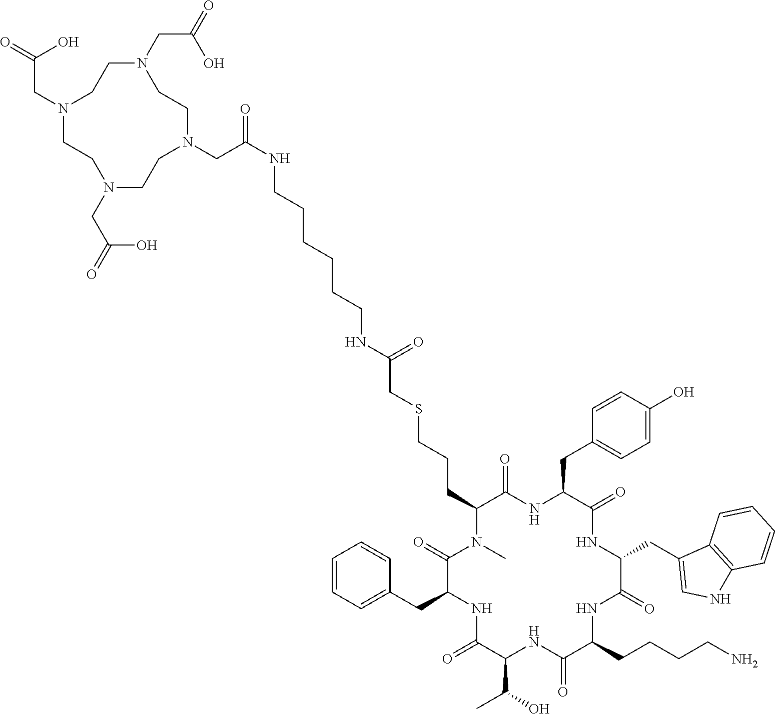 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0003485 (197) | 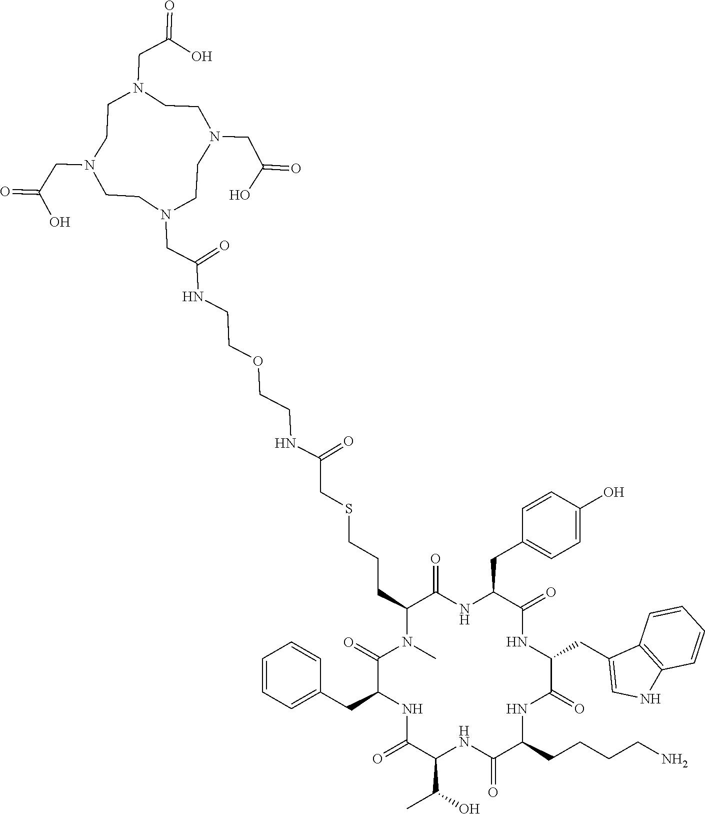 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCSCC(=O)NCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0003503 (198) | 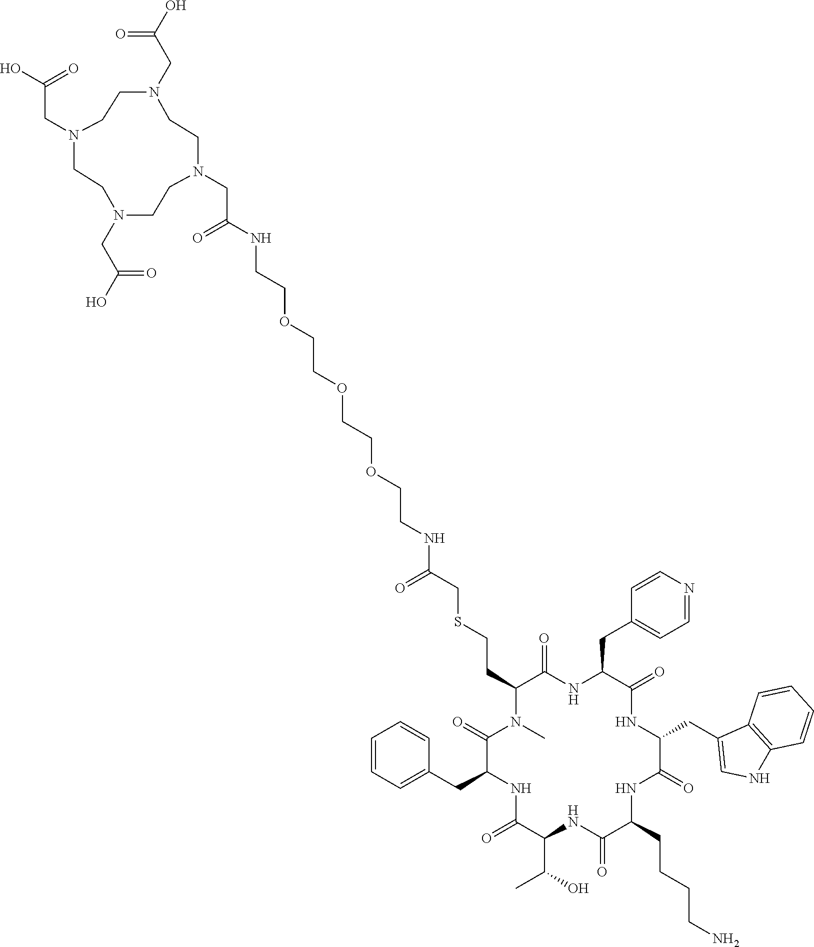 [SMILES: C[C@@H](O)[C@@H]1NC(=O)[C@H](CCCCN)NC(=O)[C@@H](Cc2c[nH]c3ccccc23)NC(=O)[C@H](Cc2ccncc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0004535 (199) | 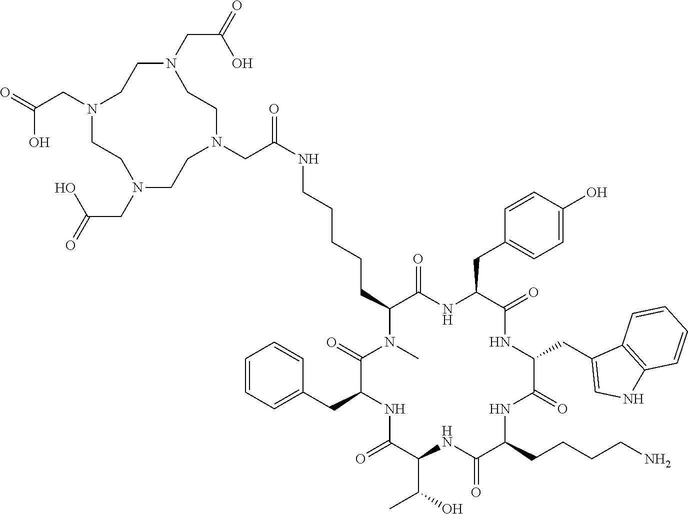 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004537 (200) | 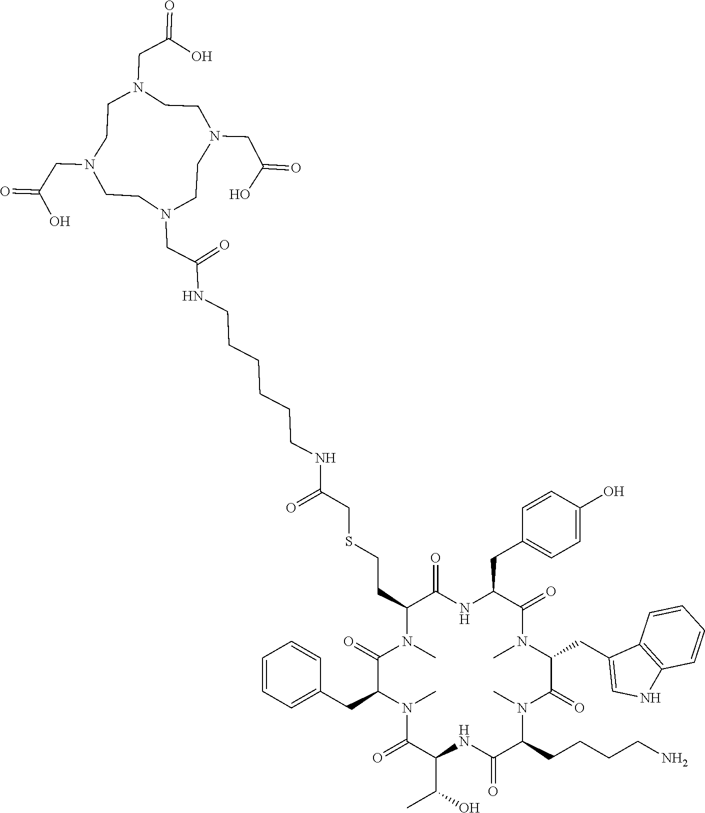 [SMILES: C[C@@H](O)[C@@H]1NC(=O)[C@H](CCCCN)N(C)C(=O)[C@@H](Cc2c[nH]c3ccccc23)N(C)C(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)N(C)C1=O] |
| RB-0004539 (201) | 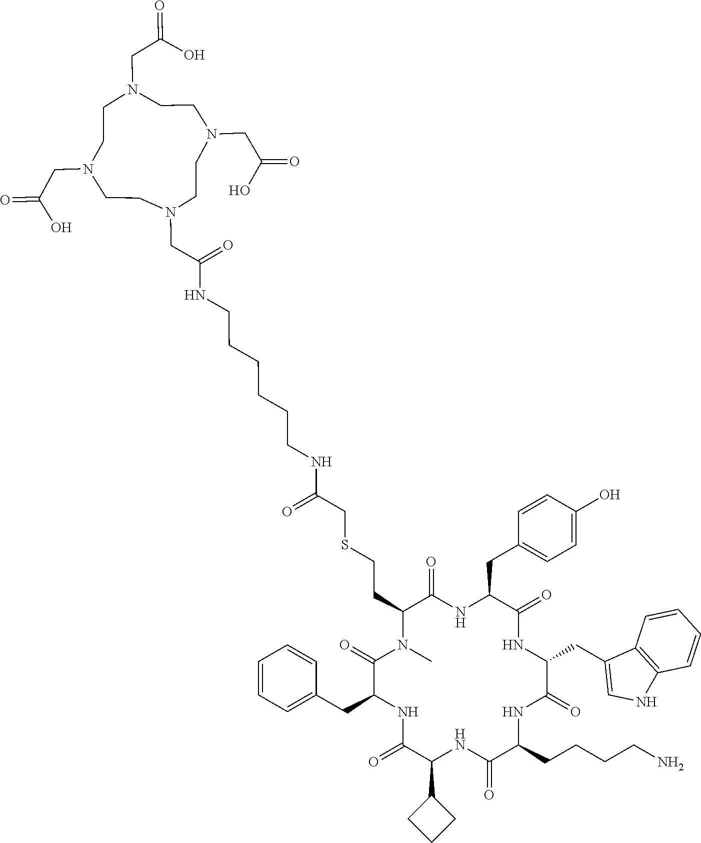 [SMILES: CN1C(=O)[C@H](Cc2ccccc2)NC(=O)[C@H](C2CCC2)NC(=O)[C@H](CCCCN)NC(=O)[C@@H](Cc2c[nH]c3ccccc23)NC(=O)[C@H](Cc2ccc(O)cc2)NC(=O)[C@@H]1CCSCC(=O)NCCCCCCNC(=O)CN1CCN(CC(=O)O)CCN(CC(=O)O)CCN(CC(=O)O)CC1] |
| RB-0004608 (202) | 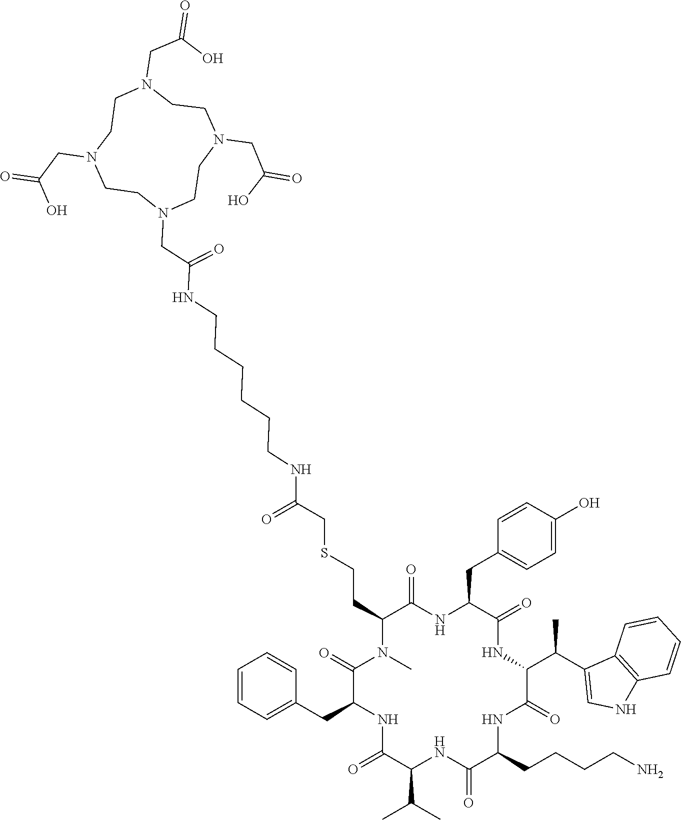 [SMILES: CC(C)[C@@H]1NC(=O)[C@H](CCCCN)NC(=O)[C@@H]([C@@H](C)c2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004610 (203) | 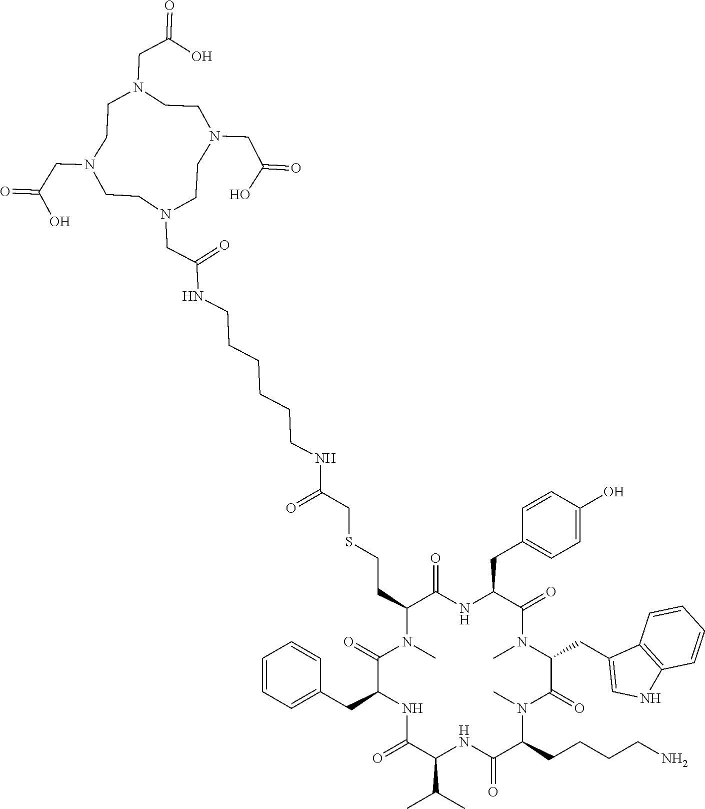 [SMILES: CC(C)[C@@H]1NC(=O)[C@H](CCCCN)N(C)C(=O)[C@@H](Cc2c[nH]c3ccccc23)N(C)C(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004612 (204) | 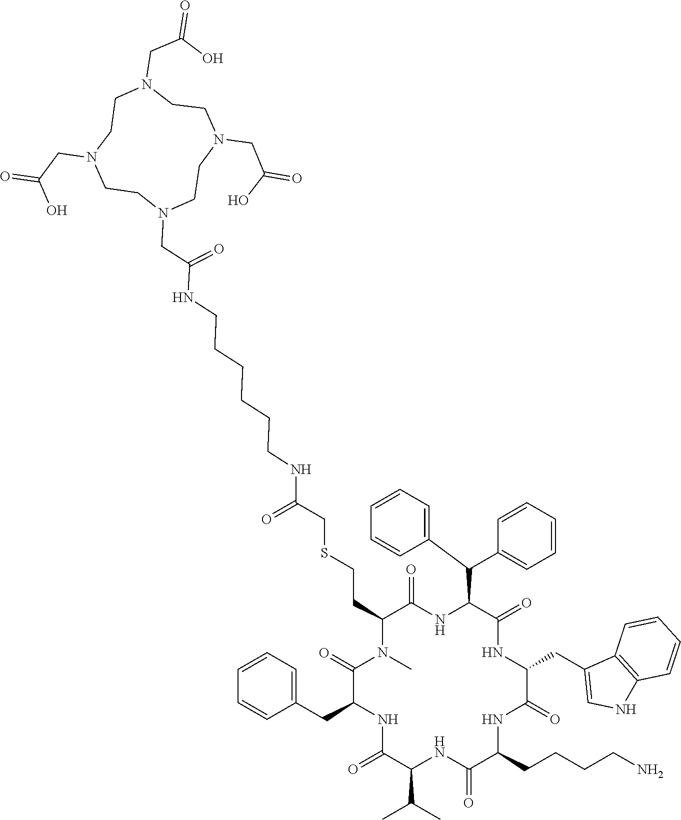 [SMILES: CC(C)[C@@H]1NC(=O)[C@H](CCCCN)NC(=O)[C@@H](Cc2c[nH]c3ccccc23)NC(=O)[C@H](C(c2ccccc2)c2ccccc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004614 (205) | 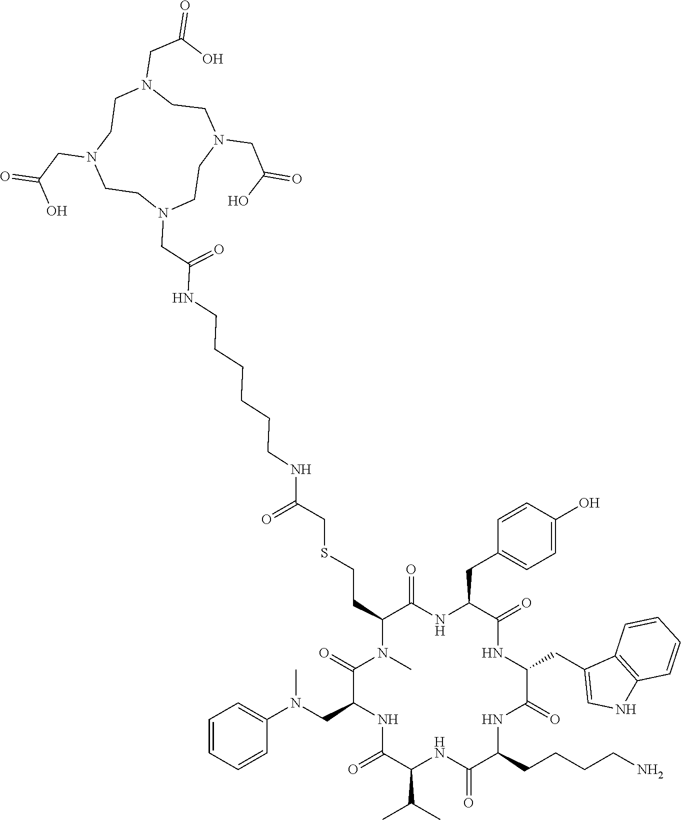 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](CN(C)c2ccccc2)NC1=O] |
| RB-0004616 (206) | 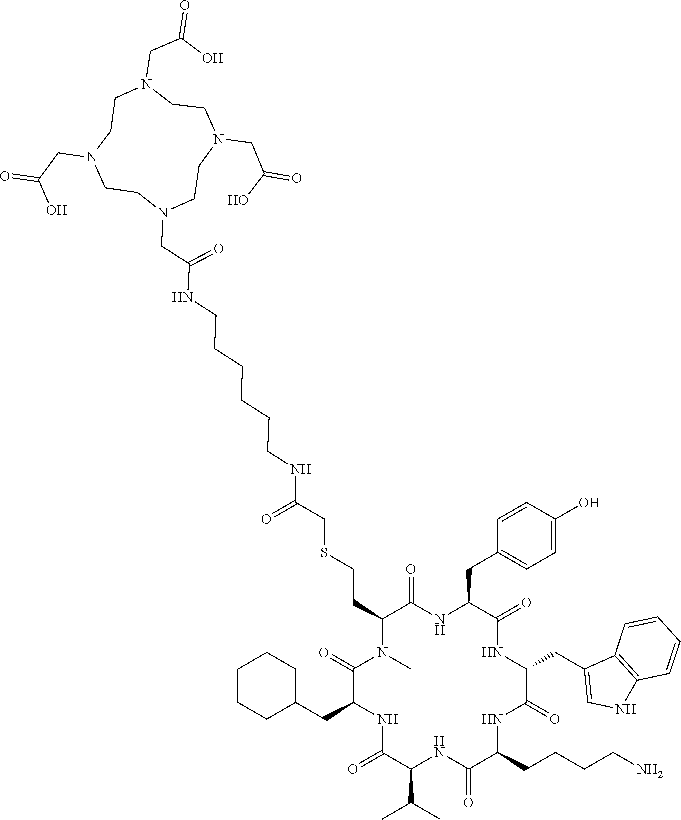 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](CC2CCCCC2)NC1=O] |
| RB-0004618 (207) | 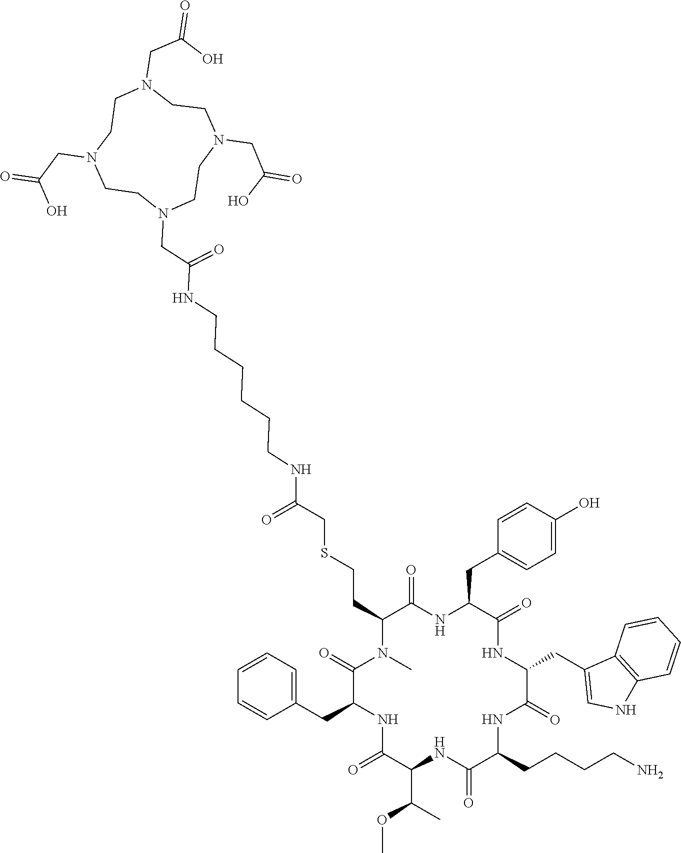 [SMILES: CO[C@H](C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004620 (208) | 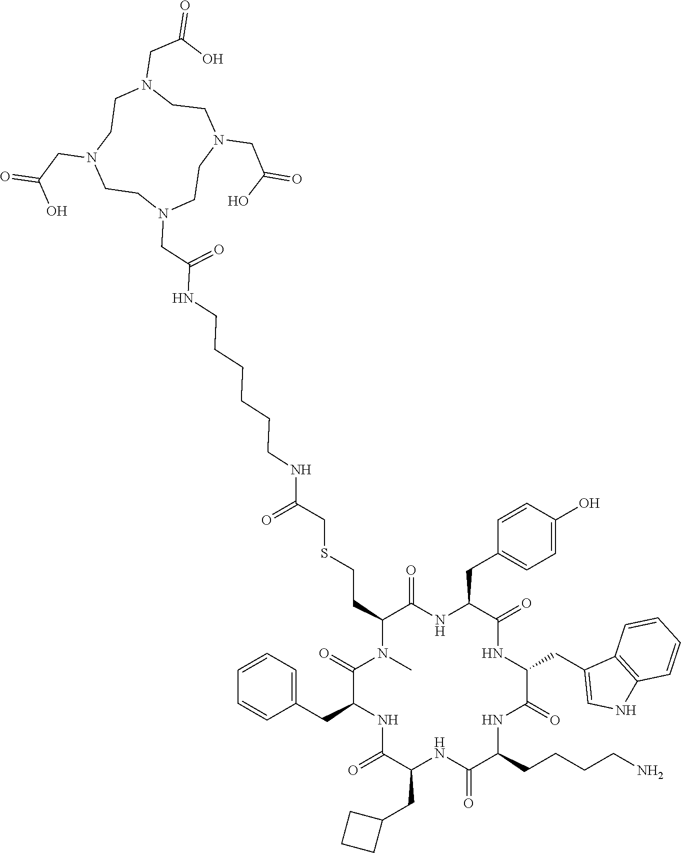 [SMILES: CN1C(=O)[C@H](Cc2ccccc2)NC(=O)[C@H](CC2CCC2)NC(=O)[C@H](CCCCN)NC(=O)[C@@H](Cc2c[nH]c3ccccc23)NC(=O)[C@H](Cc2ccc(O)cc2)NC(=O)[C@@H]1CCSCC(=O)NCCCCCCNC(=O)CN1CCN(CC(=O)O)CCN(CC(=O)O)CCN(CC(=O)O)CC1] |
| RB-0004622 (209) | 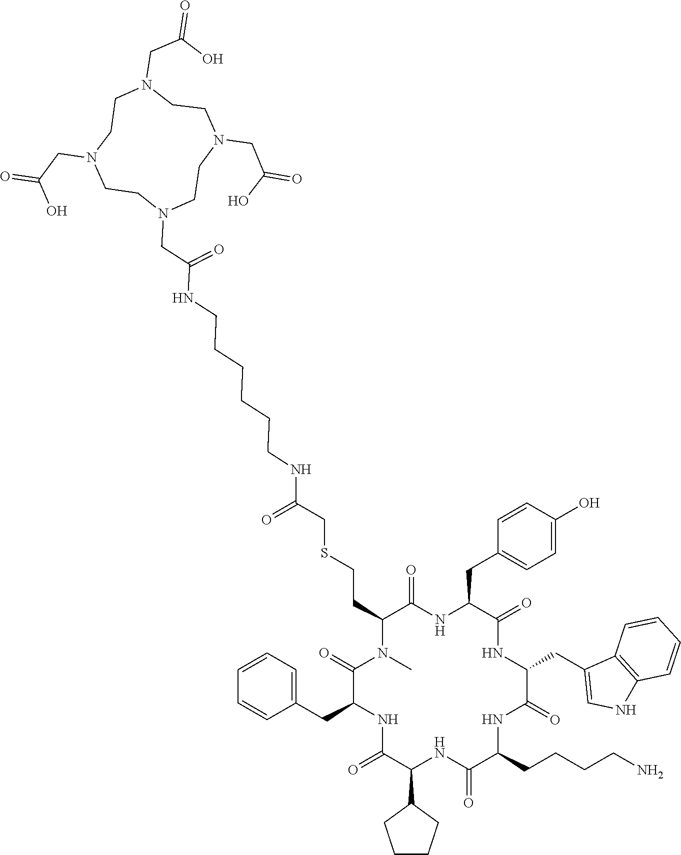 [SMILES: CN1C(=O)[C@H](Cc2ccccc2)NC(=O)[C@H](C2CCCC2)NC(=O)[C@H](CCCCN)NC(=O)[C@@H](Cc2c[nH]c3ccccc23)NC(=O)[C@H](Cc2ccc(O)cc2)NC(=O)[C@@H]1CCSCC(=O)NCCCCCCNC(=O)CN1CCN(CC(=O)O)CCN(CC(=O)O)CCN(CC(=O)O)CC1] |
| RB-0004818 (210) | 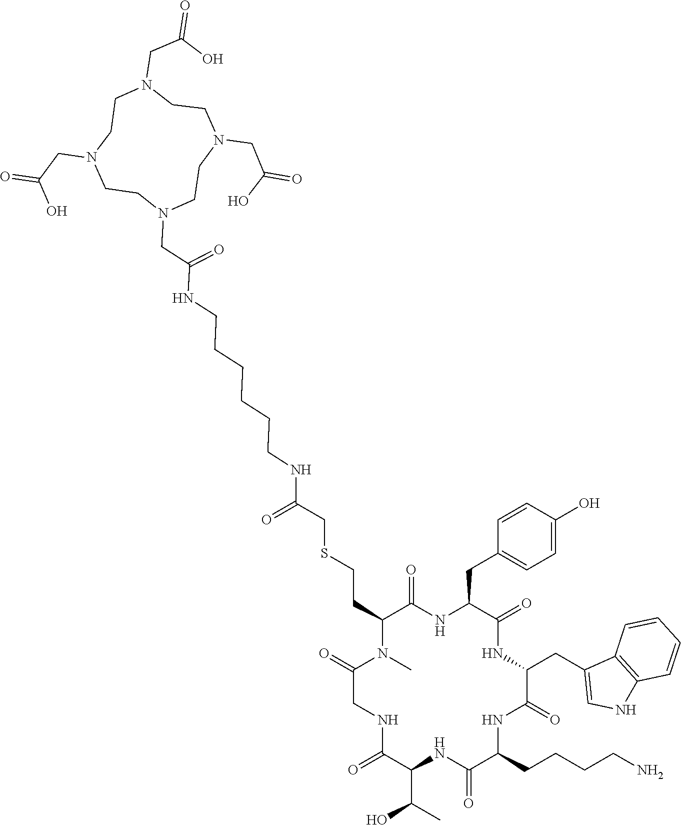 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)CNC1=O] |
| RB-0004820 (211) | 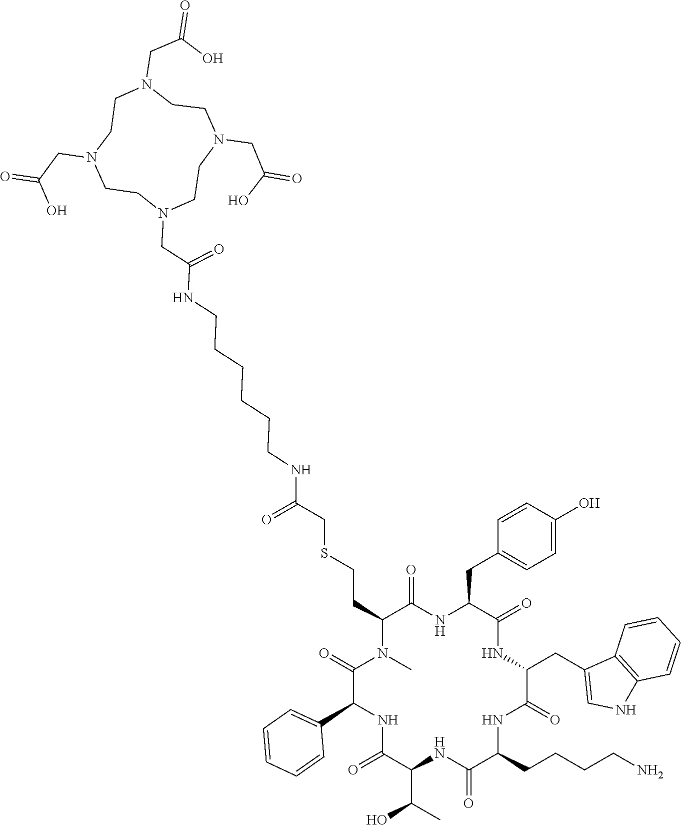 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](c2ccccc2)NC1=O] |
| RB-0004822 (212) | 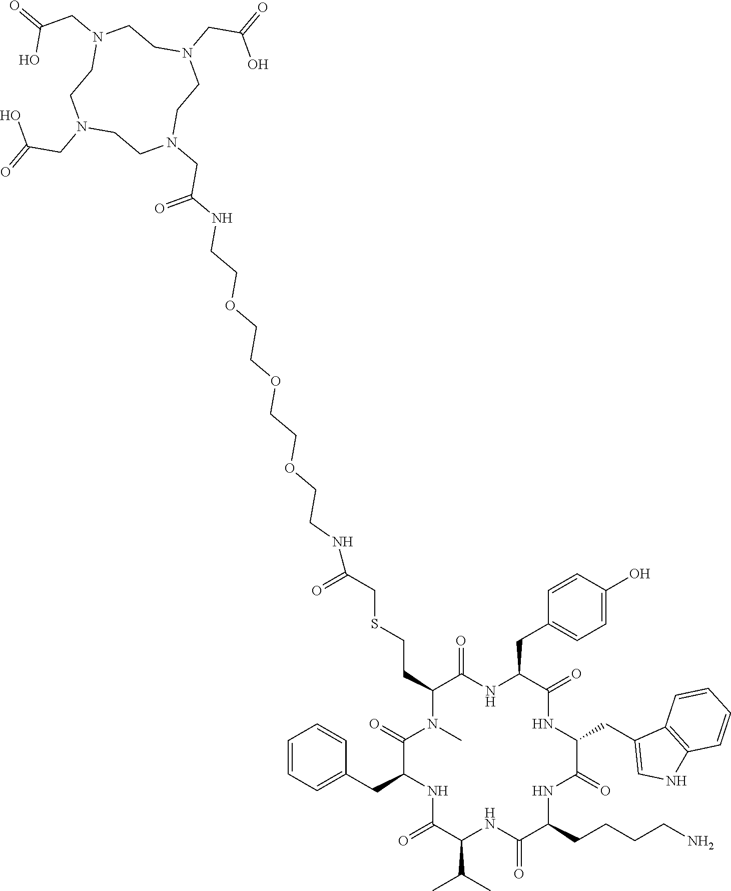 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0004824 (213) | 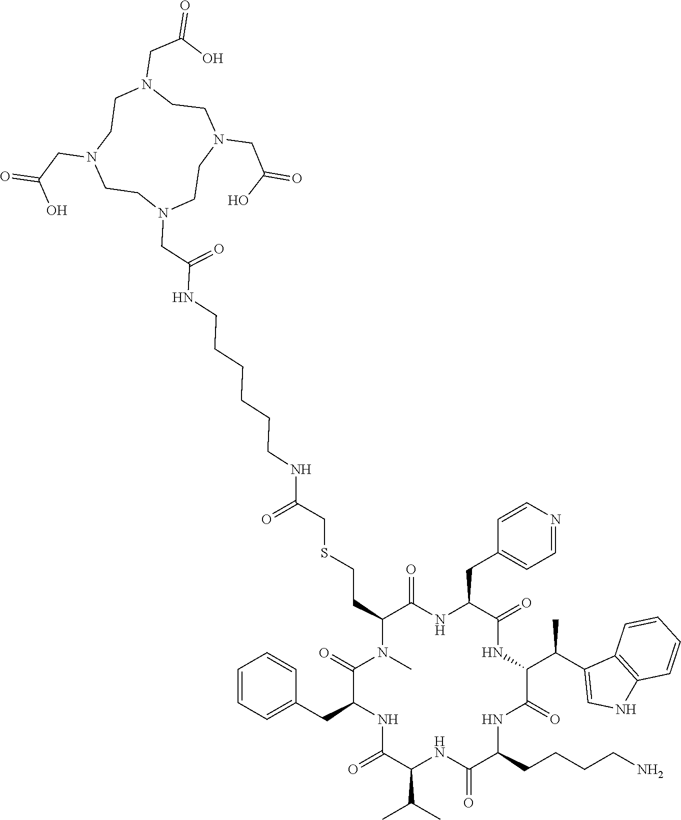 [SMILES: CC(C)[C@@H]1NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004863 (214) | 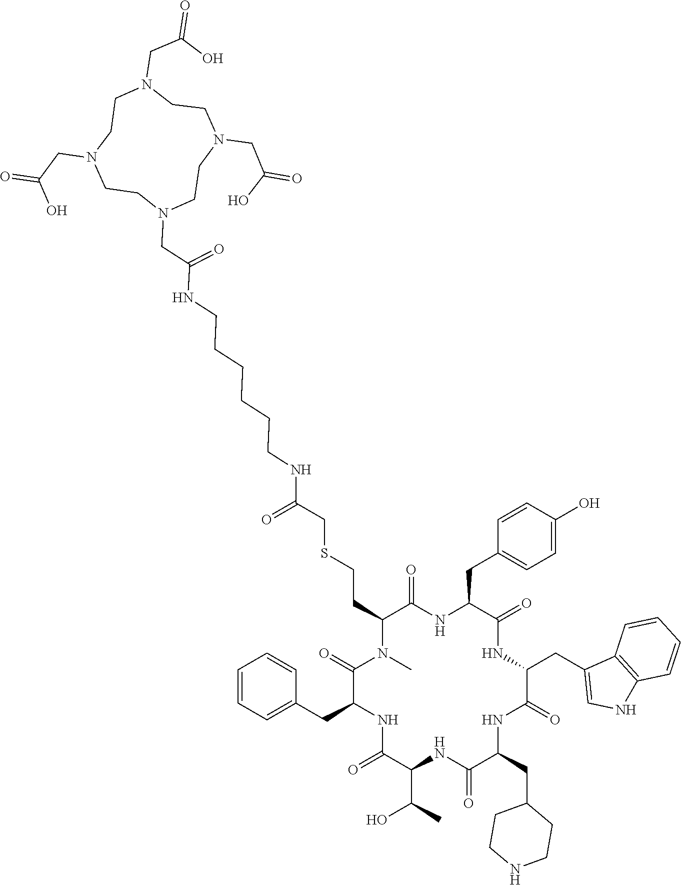 [SMILES: C[C@@H](O)[C@@H]1NC(=O)[C@H](CC2CCNCC2)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004865 (215) | 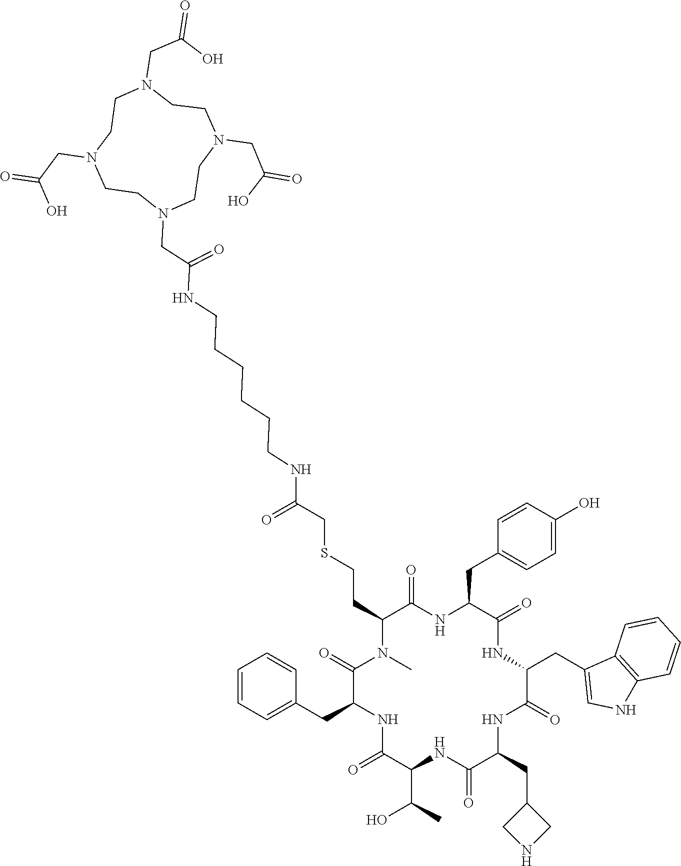 [SMILES: C[C@@H](O)[C@@H]1NC(=O)[C@H](CC2CNC2)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0004867 (216) | 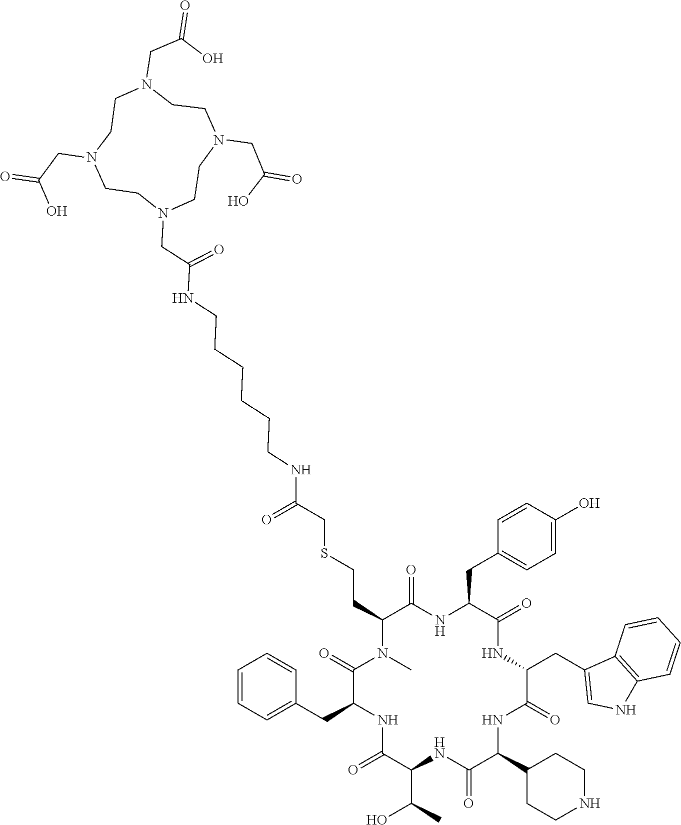 [SMILES: C[C@@H](O)[C@@H]1NC(=O)[C@H](C2CCNCC2)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005246 (217) | 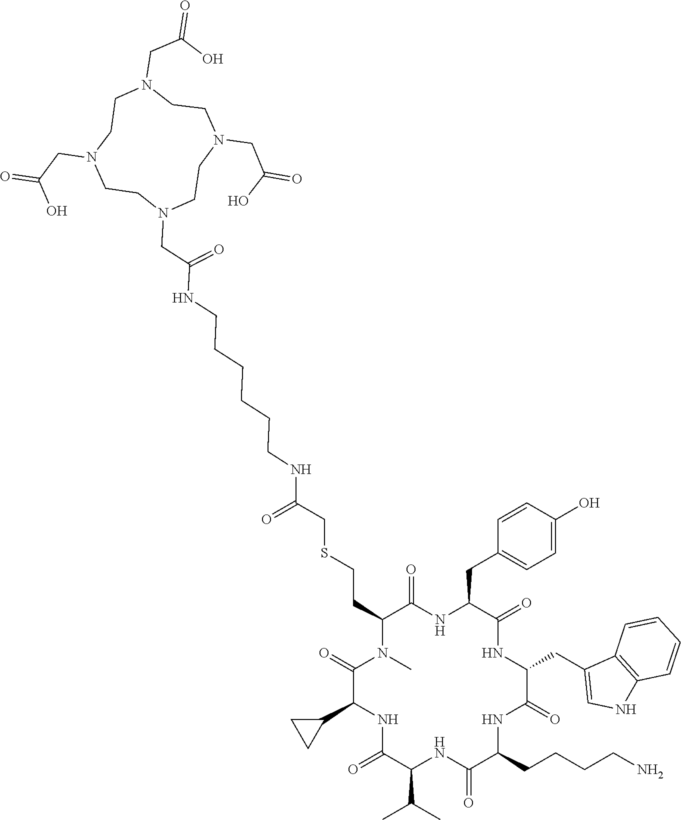 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](C2CC2)NC1=O] |
| RB-0005252 (218) | 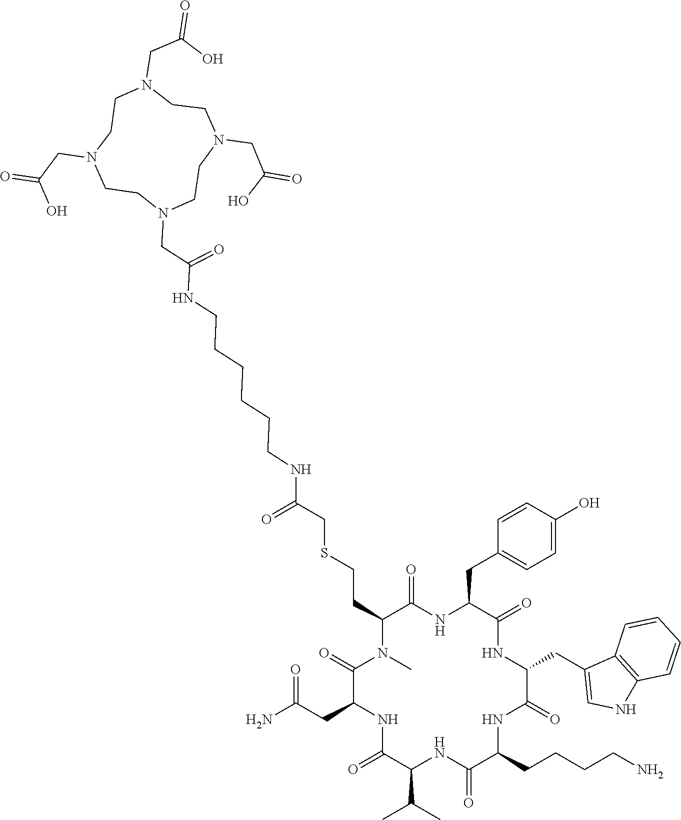 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](CC(N)=O)NC1=O] |
| RB-0005254 (219) | 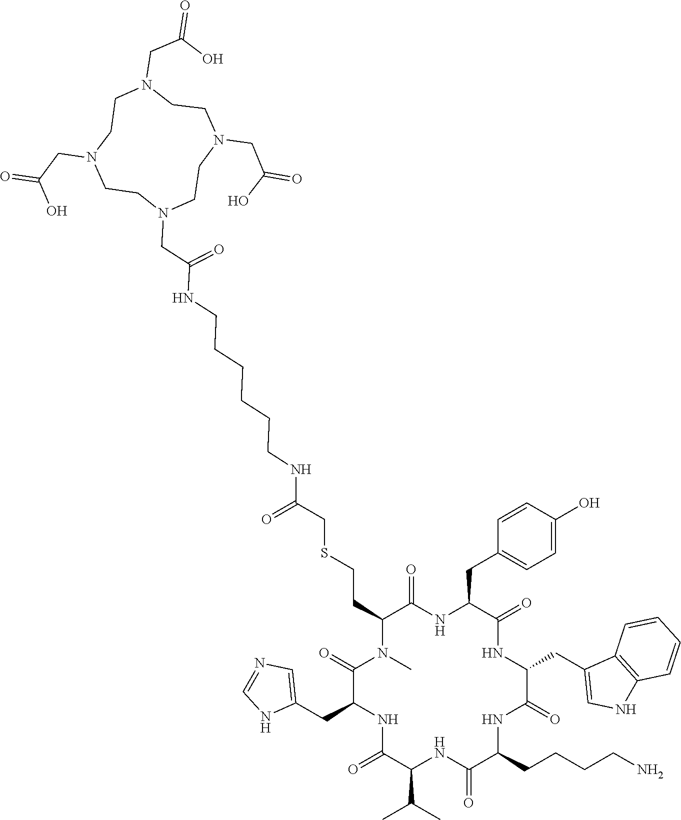 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2cnc[nH]2)NC1=O] |
| RB-0005265 (220) | 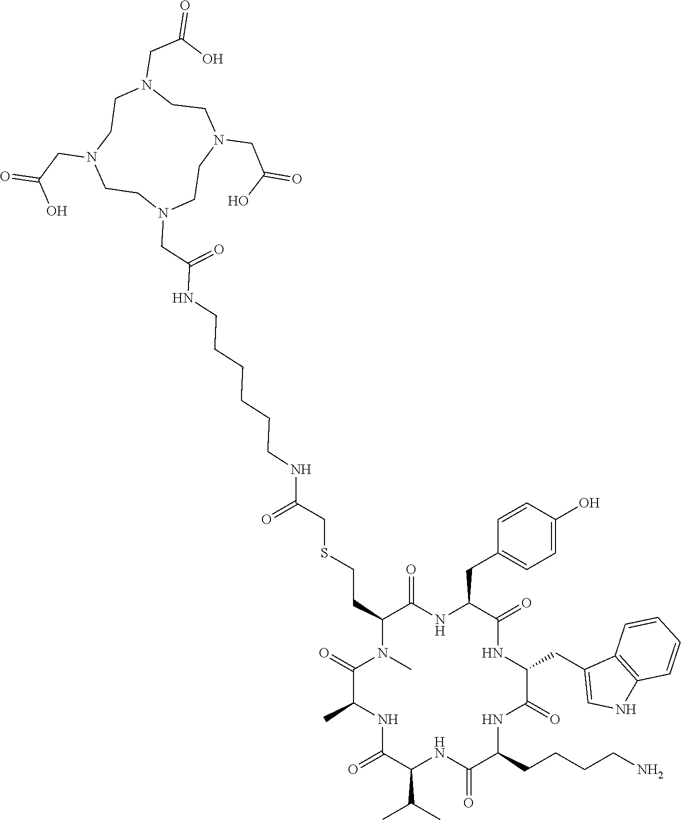 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](C)NC1=O] |
| RB-0005269 (221) | 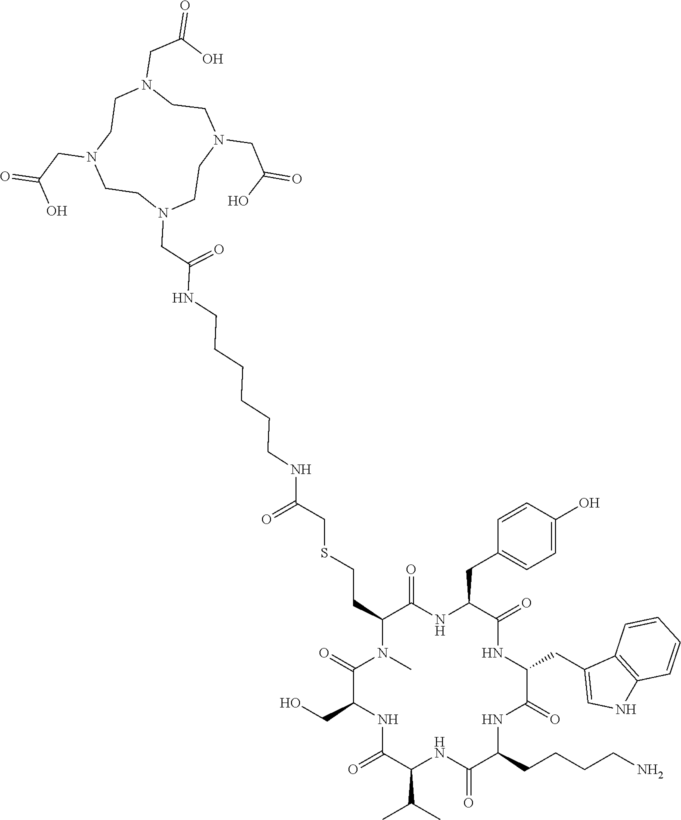 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](CO)NC1=O] |
| RB-0005271 (222) | 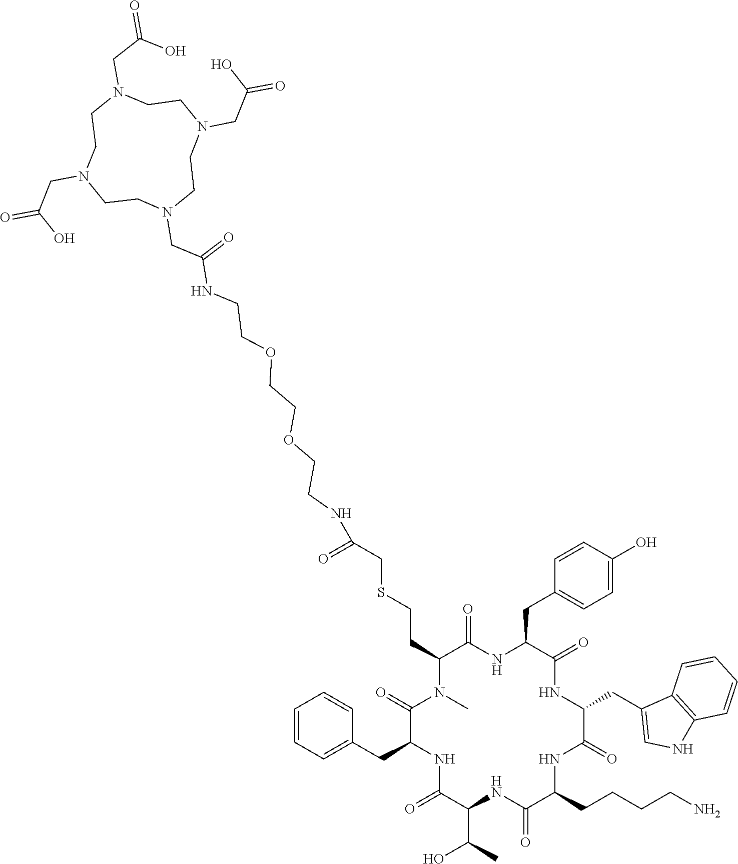 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0005273 (223) | 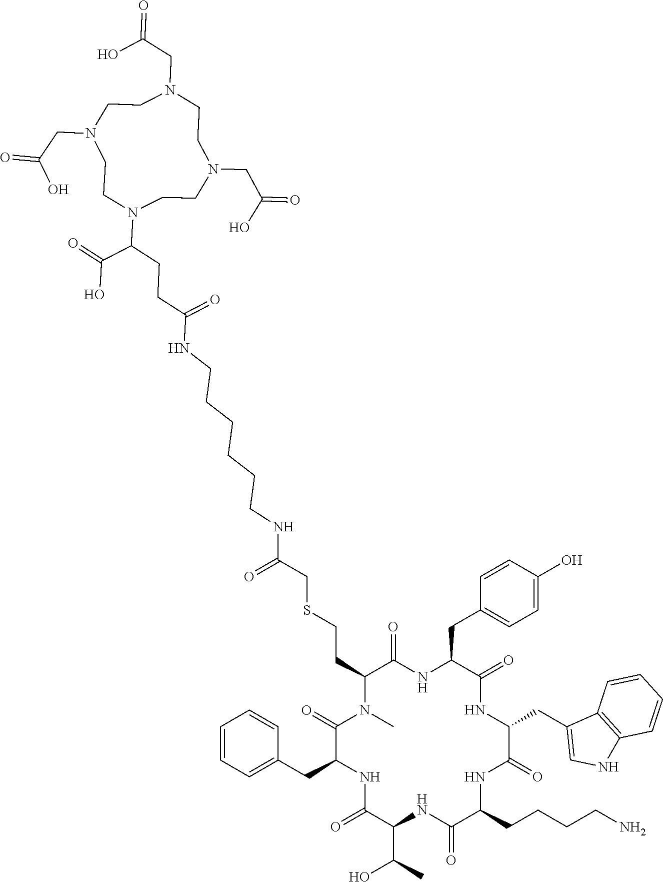 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CCC(C(=O)O)N2CCN(CC(=O)O)CCN(CC(=O)O)CCN(CC(=O)O)CC2)N(C)C(=O)[C@H](Cc2ccccc2)NC1=O] |
| RB-0005275 (224) | 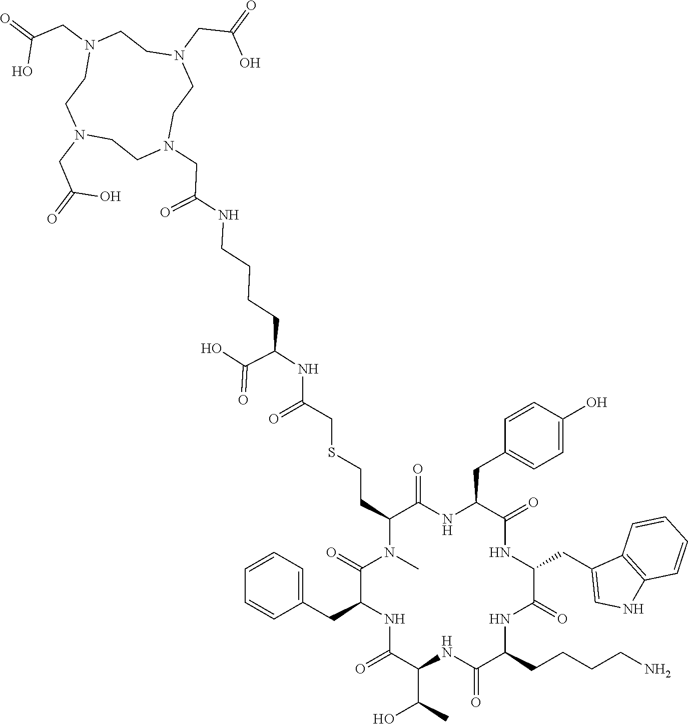 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)N[C@H](CCCCNC(=O)CN2CCN(CC(=O)O)CCN(CC(=O)O)CCN(CC(=O)O)CC2)C(=O)O)N(C)C(=O)[C@H](Cc2ccccc2)NC1=O] |
| RB-0005277 (225) | 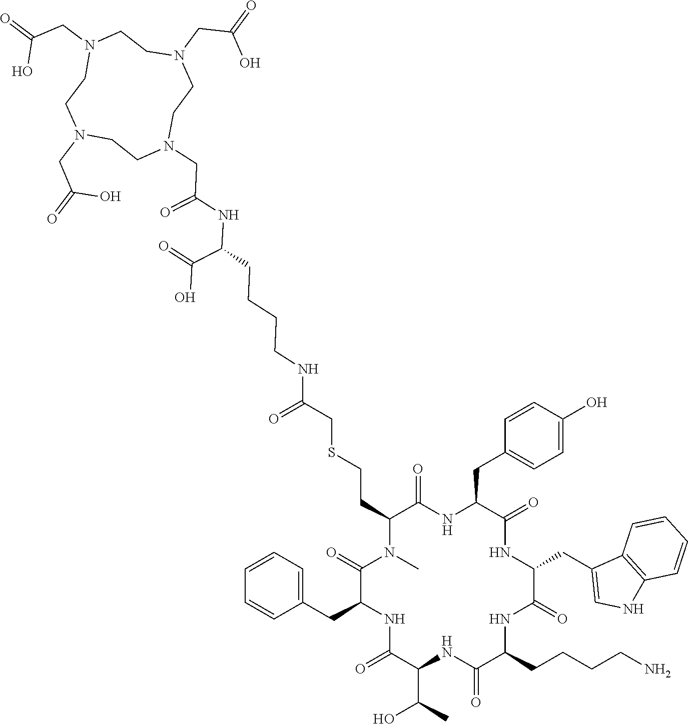 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCC[C@@H](NC(=O)CN2CCN(CC(=O)O)CCN(CC(=O)O)CCN(CC(=O)O)CC2)C(=O)O)N(C)C(=O)[C@H](Cc2ccccc2)NC1=O] |
| RB-0005283 (226) | 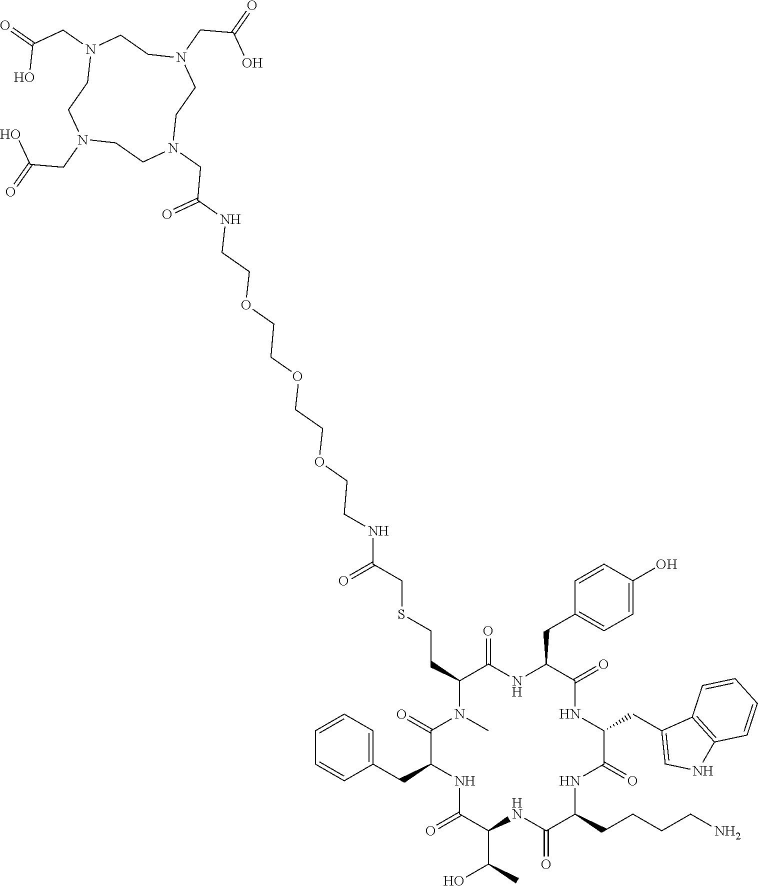 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0005285 (227) | 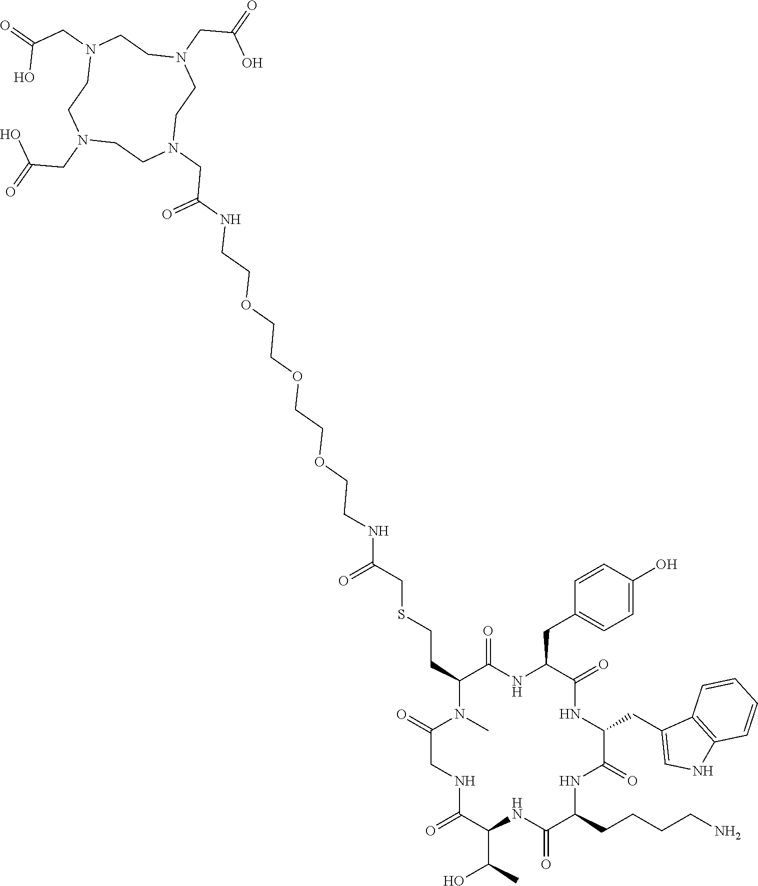 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)CNC1=O] |
| RB-0005331 (228) | 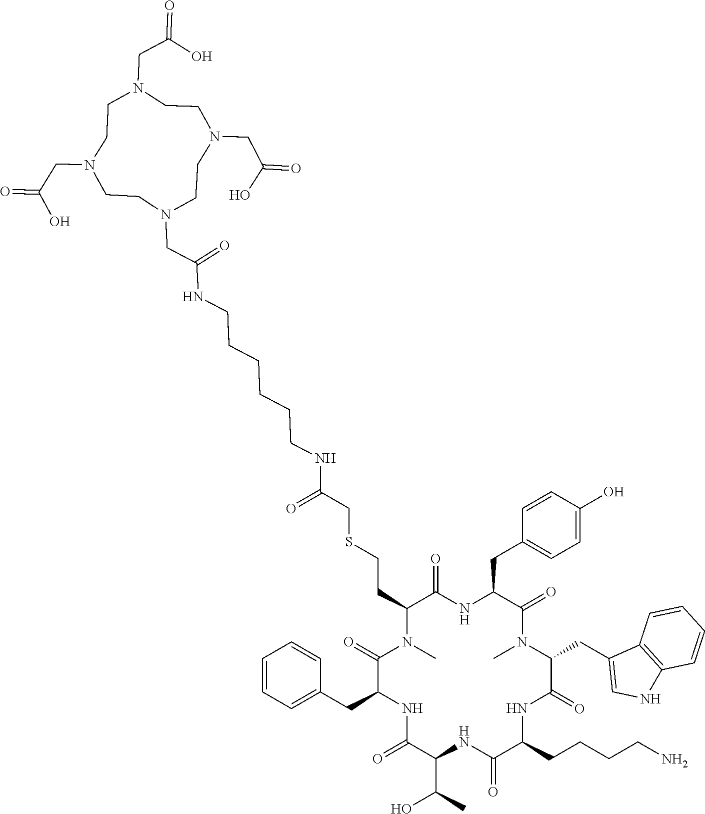 [SMILES: C[C@@H](O)[C@@H]1NC(=O)[C@H](CCCCN)NC(=O)[C@@H](Cc2c[nH]c3ccccc23)N(C)C(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005501 (229) | 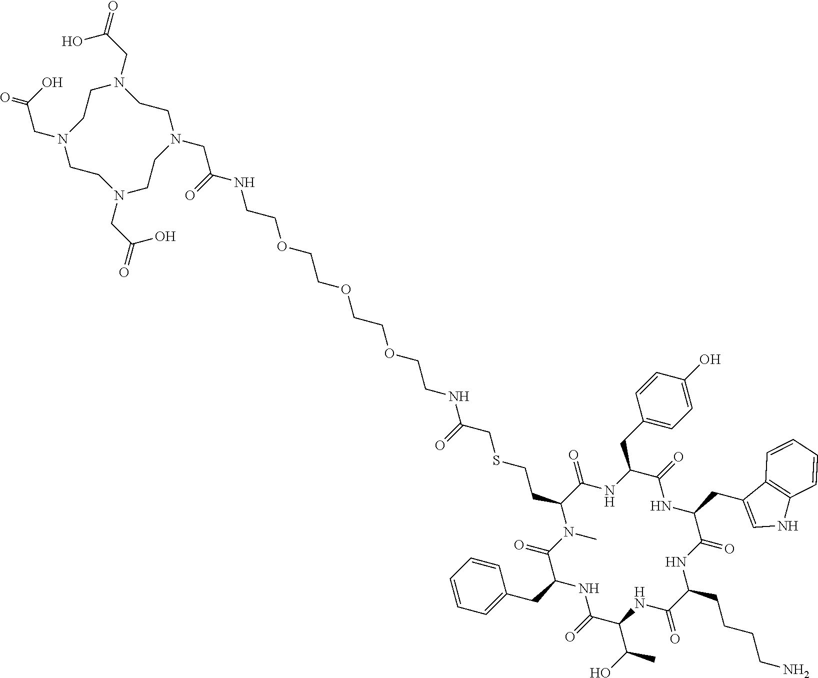 [SMILES: C[C@@H](O)[C@@H]1NC(=O)[C@H](CCCCN)NC(=O)[C@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0005691 (230) | 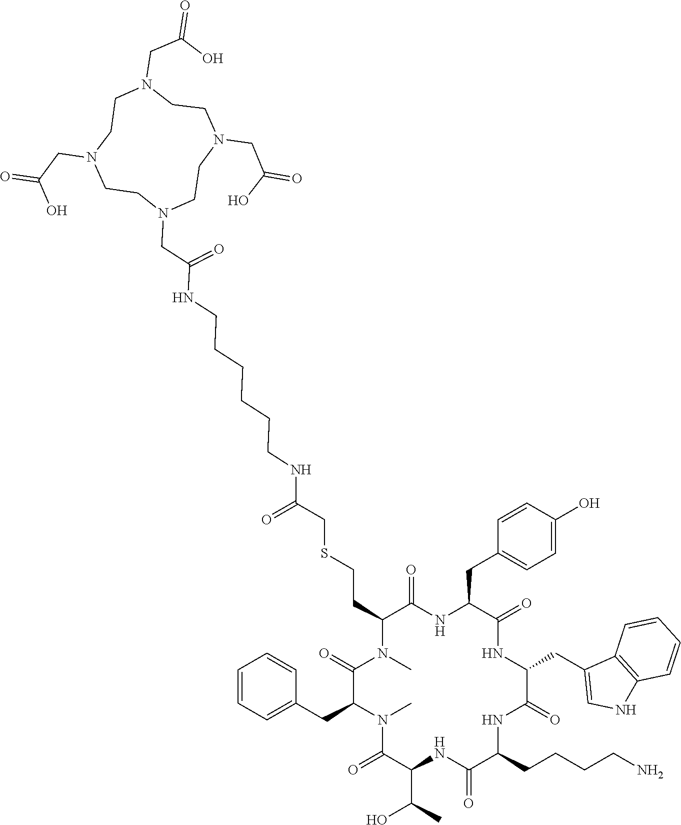 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)N(C)C1=O] |
| RB-0005693 (231) | 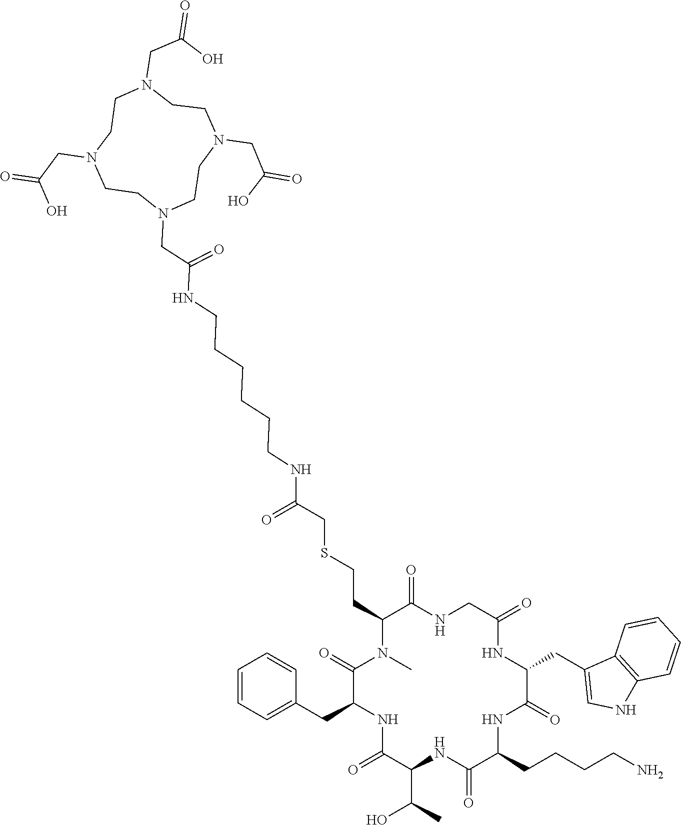 [SMILES: C[C@@H](O)[C@@H]1NC(=O)[C@H](CCCCN)NC(=O)[C@@H](Cc2c[nH]c3ccccc23)NC(=O)CNC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005712 (214) | 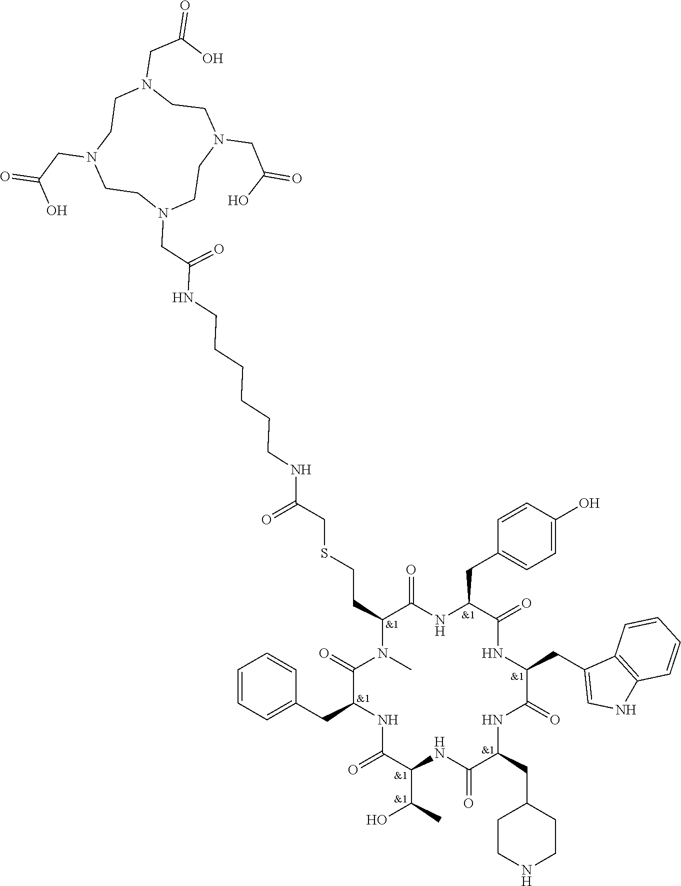 [SMILES: C[C@@H](O)[C@@H]1NC(=O)[C@H](CC2CCNCC2)NC(=O)[C@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005716 (233) | 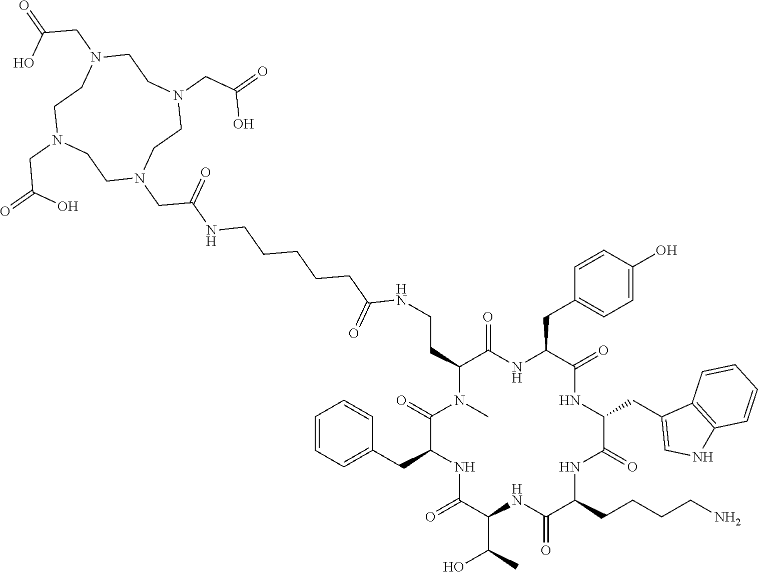 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCNC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005727 (234) | 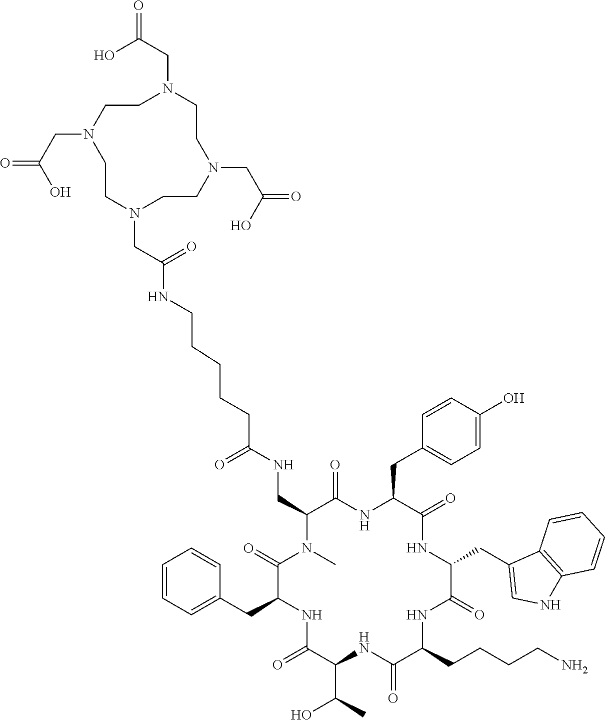 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CNC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005729 (235) | 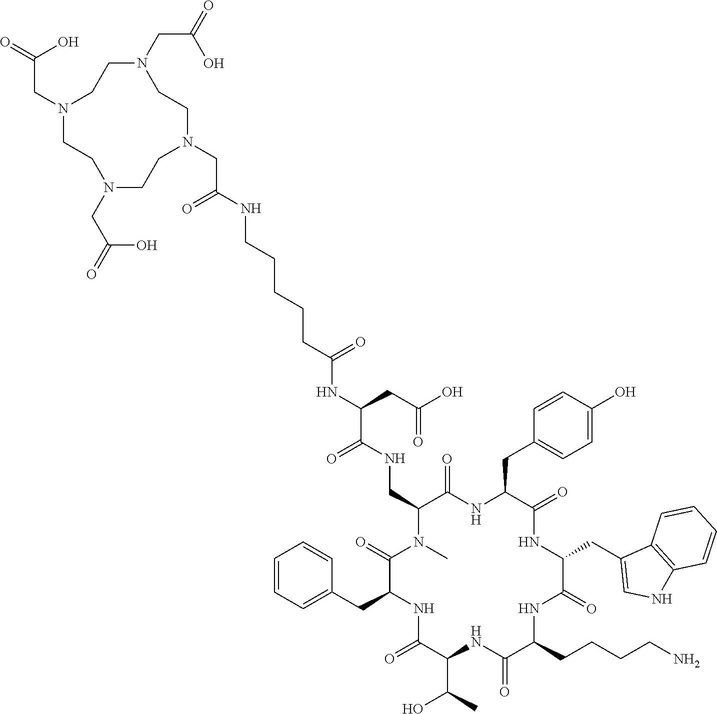 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CNC(=O)[C@H](CC(=O)O)NC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005731 (236) | 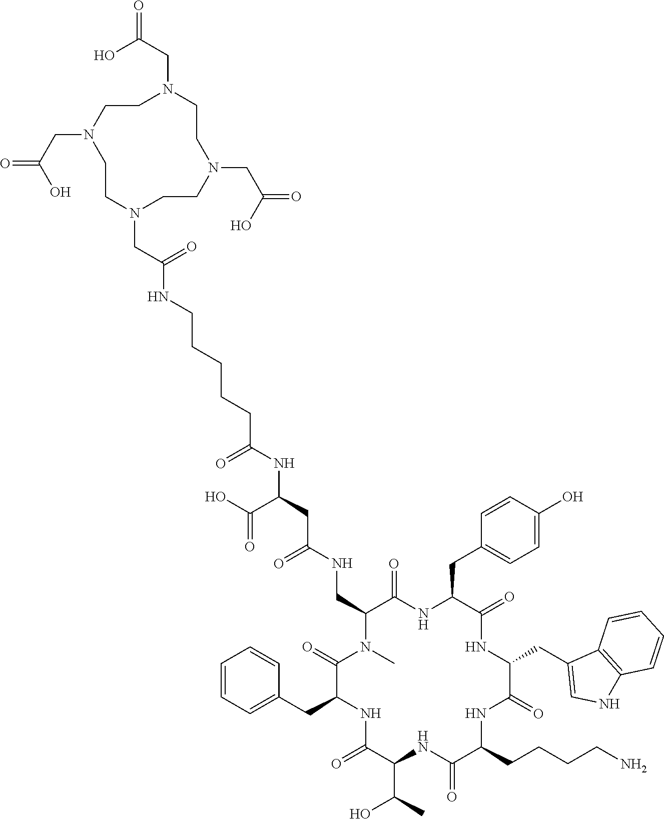 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CNC(=O)C[C@H](NC(=O)CCCCCNC(=O)CN2CCN(CC(=O)O)CCN(CC(=O)O)CCN(CC(=O)O)CC2)C(=O)O)N(C)C(=O)[C@H](Cc2ccccc2)NC1=O] |
| RB-0005733 (237) | 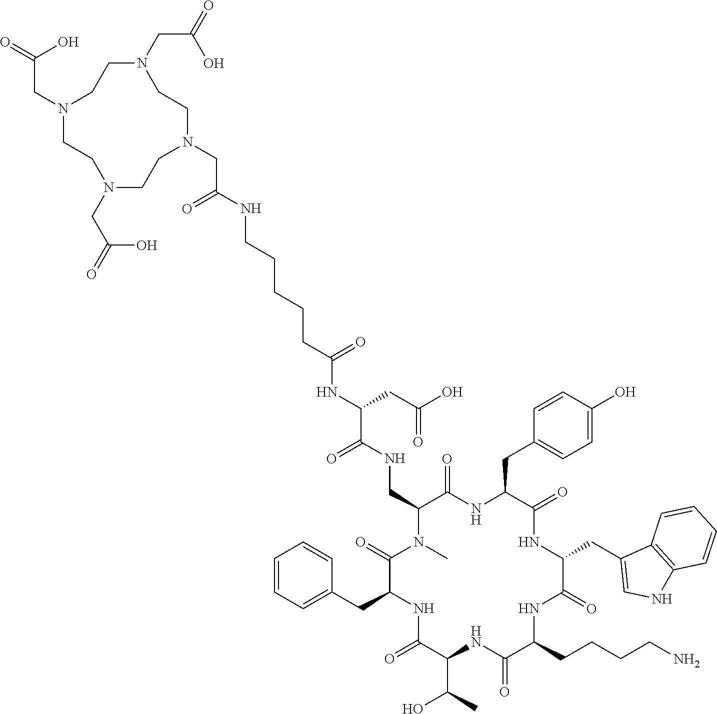 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CNC(=O)[C@@H](CC(=O)O)NC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005735 (238) | 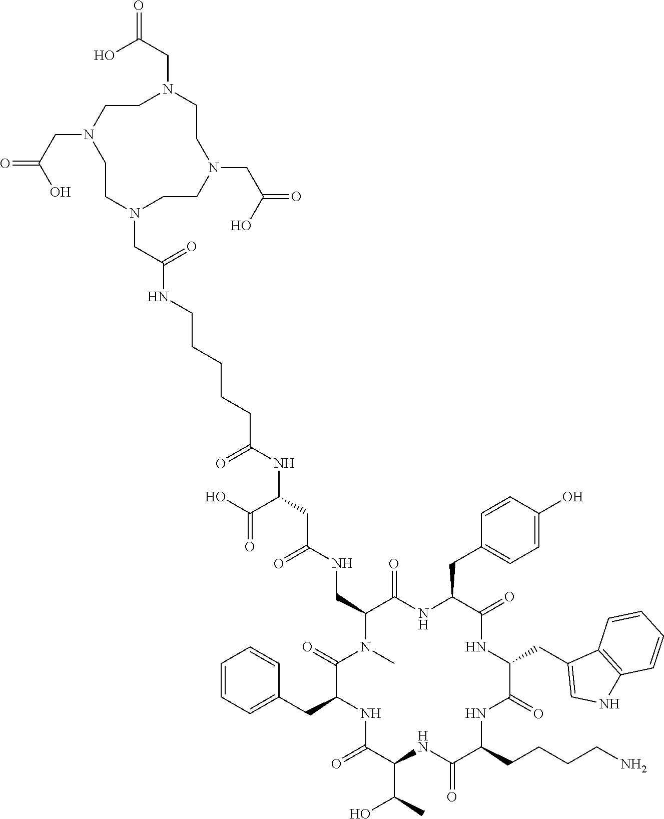 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CNC(=O)C[C@@H](NC(=O)CCCCCNC(=O)CN2CCN(CC(=O)O)CCN(CC(=O)O)CCN(CC(=O)O)CC2)C(=O)O)N(C)C(=O)[C@H](Cc2ccccc2)NC1=O] |
| RB-0005737 (239) | 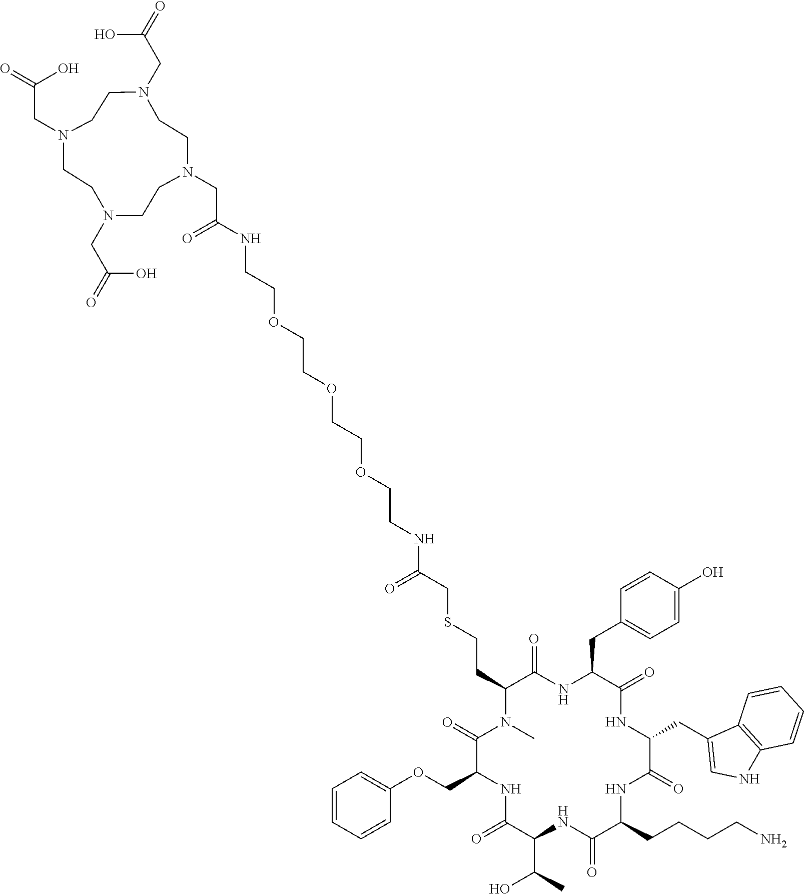 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](COc2ccccc2)NC1=O] |
| RB-0005739 (240) | 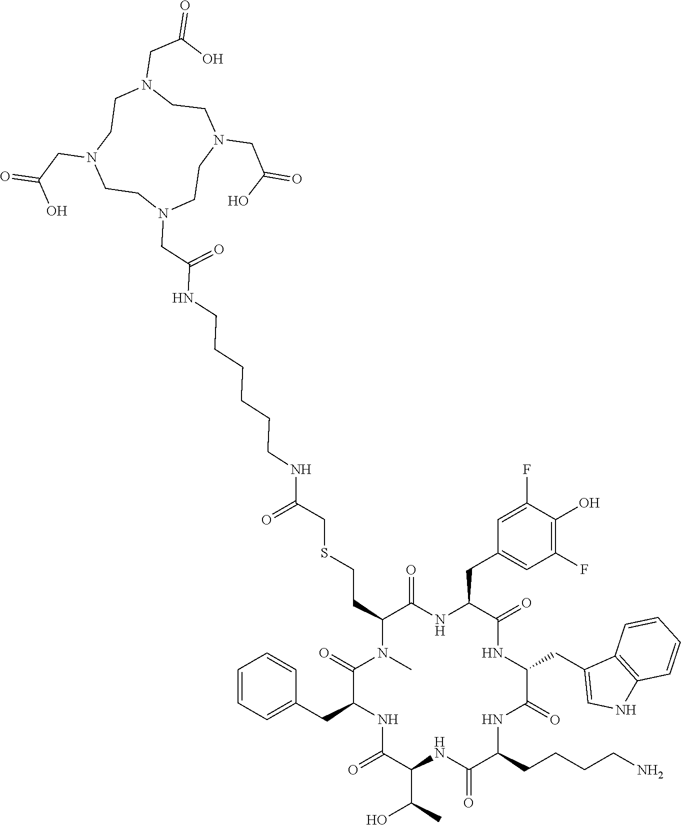 [SMILES: C[C@@H](O)[C@@H]1NC(=O)[C@H](CCCCN)NC(=O)[C@@H](Cc2c[nH]c3ccccc23)NC(=O)[C@H](Cc2cc(F)c(O)c(F)c2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005743 (241) | 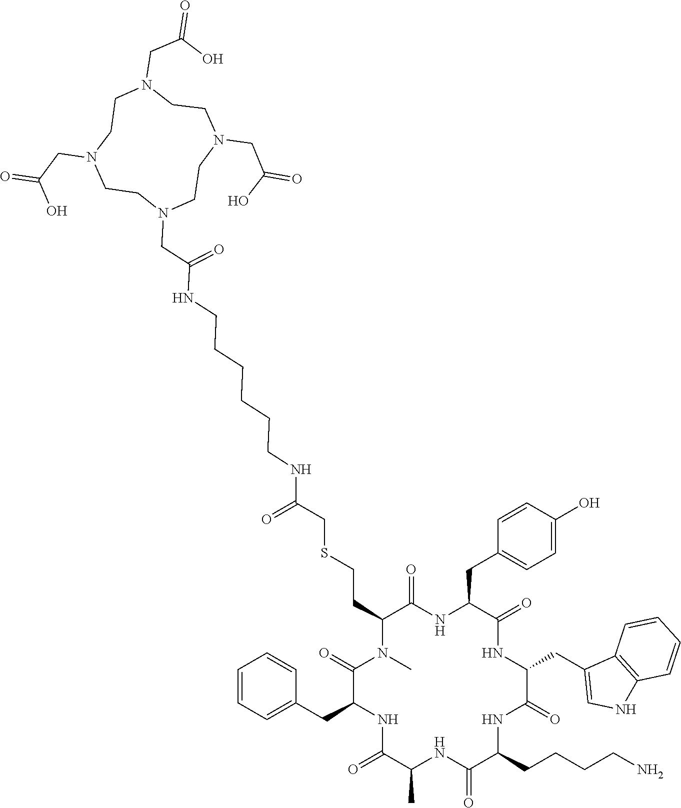 [SMILES: C[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005796 (244) | 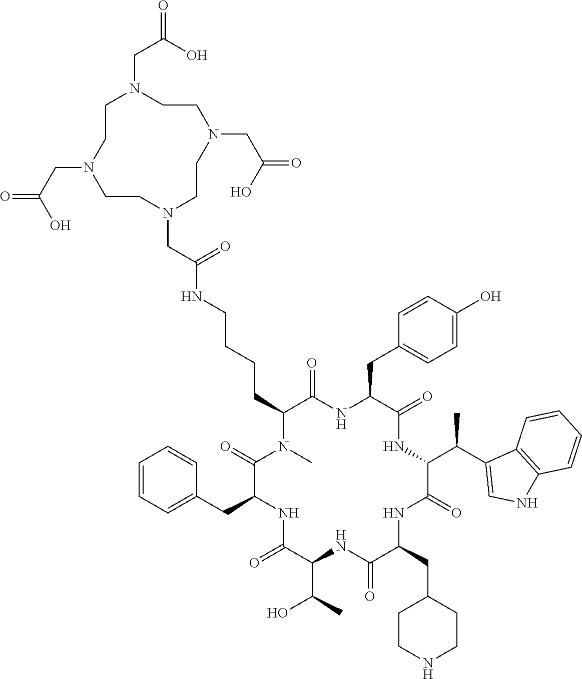 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005798 (245) | 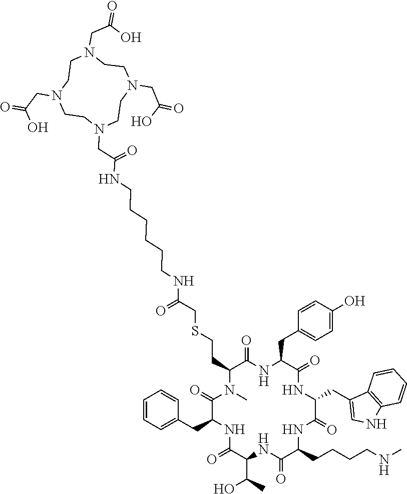 [SMILES: CNCCCC[C@@H]1NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC(=O)[C@H]([C@@H](C)O)NC1=O] |
| RB-0005800 (246) | 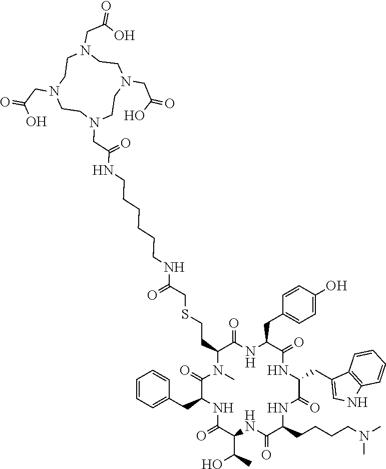 [SMILES: C[C@@H](O)[C@@H]1NC(=O)[C@H](CCCCN(C)C)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005804 (247) | 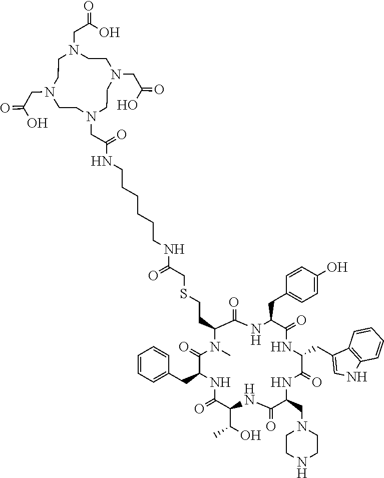 [SMILES: C[C@@H](O)[C@@H]1NC(=O)[C@H](CN2CCNCC2)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005806 (248) | 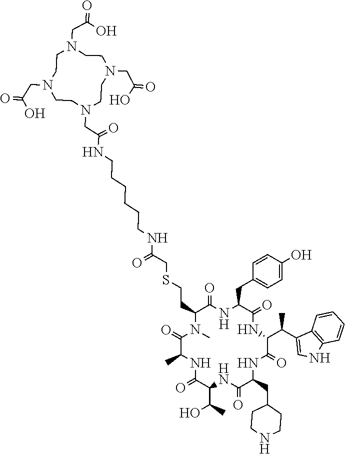 [SMILES: C[C@@H]1NC(=O)[C@H]([C@@H](C)O)NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C1=O] |
| RB-0005808 (249) | 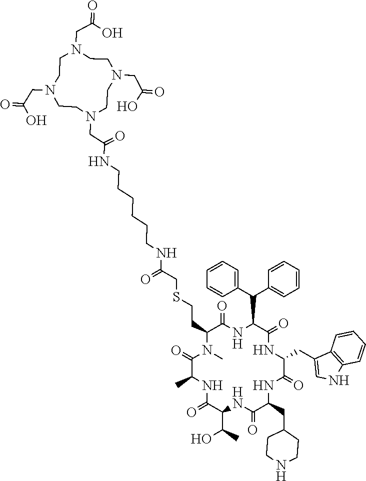 [SMILES: C[C@@H]1NC(=O)[C@H]([C@@H](C)O)NC(=O)[C@H](CC2CCNCC2)NC(=O)[C@@H](Cc2c[nH]c3ccccc23)NC(=O)[C@H](C(c2ccccc2)c2ccccc2)NC(=O)[C@H](CCSCC(=O)NCCCCCCNC(=O)CN2CCN(CC(=O)O)CCN(CC(=O)O)CCN(CC(=O)O)CC2)N(C)C1=O] |
| RB-0005826 (250) | 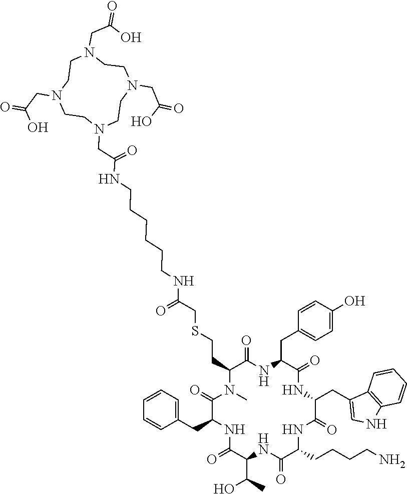 [SMILES: C[C@@H](O)[C@@H]1NC(=O)[C@@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005851 (251) | 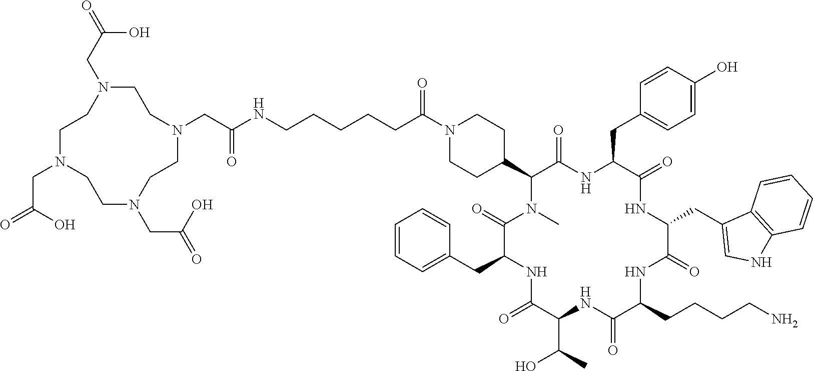 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](C2CCN(C(=O)CCCCCNC(=O)CN3CCN(CC(=O)O)CCN(CC(=O)O)CCN(CC(=O)O)CC3)CC2)N(C)C(=O)[C@H](Cc2ccccc2)NC1=O] |
| RB-0005853 (252) | 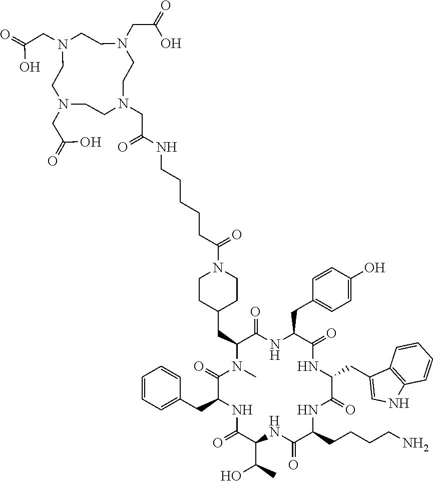 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CC2CCN(C(=O)CCCCCNC(=O)CN3CCN(CC(=O)O)CCN(CC(=O)O)CCN(CC(=O)O)CC3)CC2)N(C)C(=O)[C@H](Cc2ccccc2)NC1=O] |
| RB-0005857 (253) | 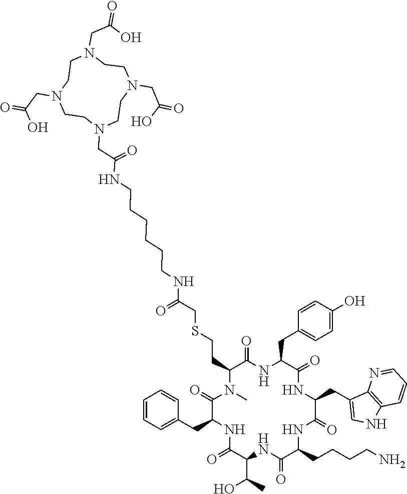 [SMILES: C[C@@H](O)[C@@H]1NC(=O)[C@H](CCCCN)NC(=O)[C@H](Cc2c[nH]c3cccnc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005863 (254) | 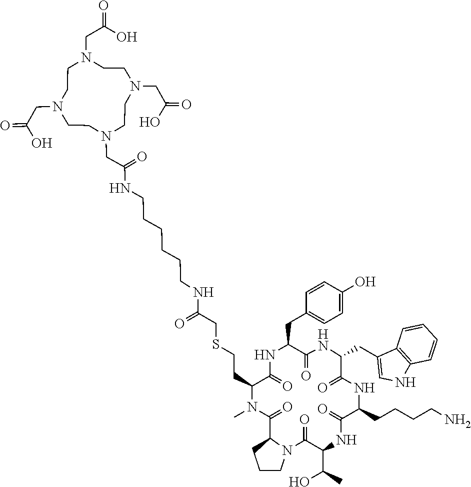 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@@H]2CCCN2C1=O] |
| RB-0005865 (255) | 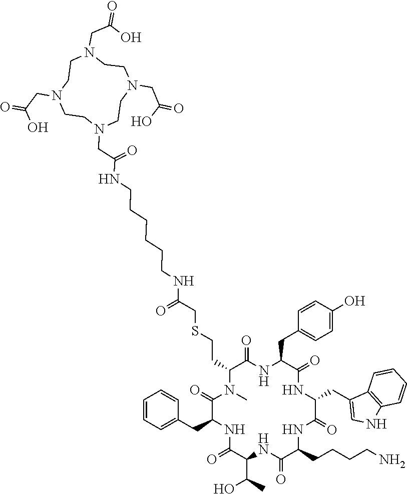 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005894 (256) | 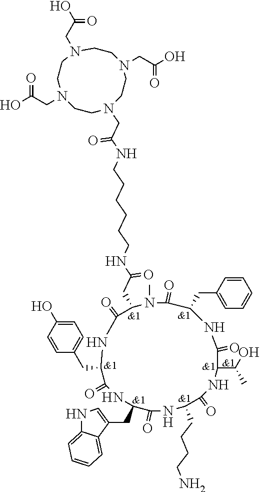 [SMILES: C[C@@H](O)C1NC(=O)[C@H](CCCCN)NC(=O)[C@@H](Cc2c[nH]c3ccccc23)NC(=O)[C@H](Cc2ccc(O)cc2)NC(=O)[C@H](CC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005896 (257) | 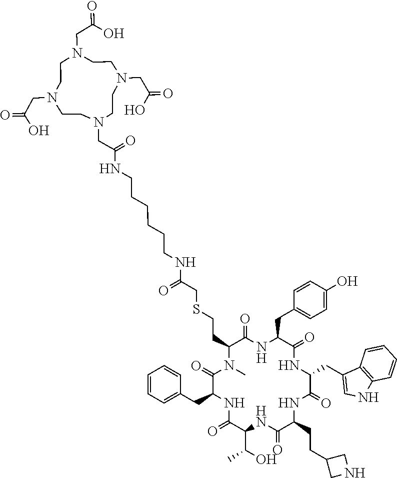 [SMILES: C[C@@H](O)[C@@H]1NC(=O)[C@H](CCC2CNC2)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005898 (258) | 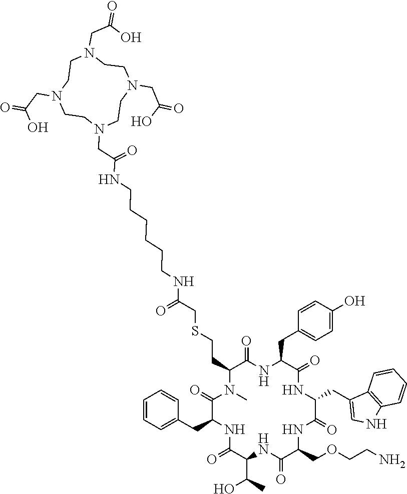 [SMILES: C[C@@H](O)[C@@H]1NC(=O)[C@H](COCCN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005902 (259) | 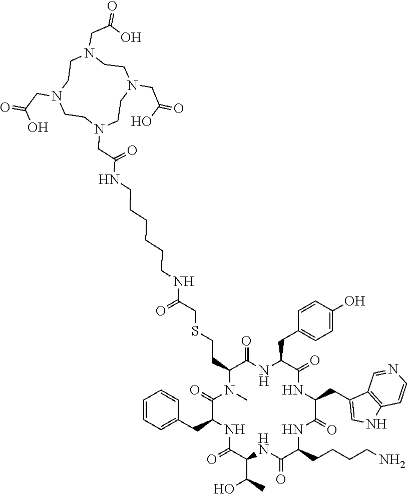 [SMILES: C[C@@H](O)[C@@H]1NC(=O)[C@H](CCCCN)NC(=O)[C@H](Cc2c[nH]c3ccncc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005904 (260) | 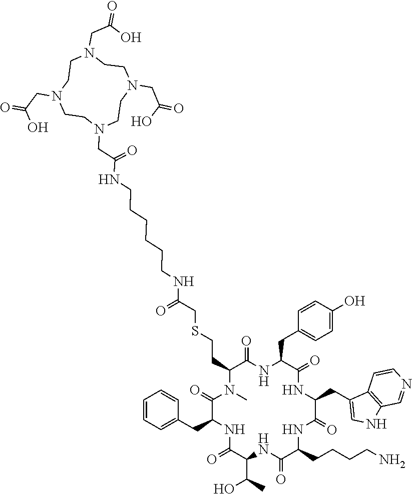 [SMILES: C[C@@H](O)[C@@H]1NC(=O)[C@H](CCCCN)NC(=O)[C@H](Cc2c[nH]c3cnccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005906 (261) | 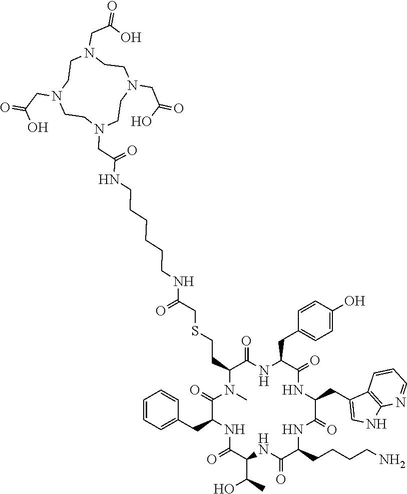 [SMILES: C[C@@H](O)[C@@H]1NC(=O)[C@H](CCCCN)NC(=O)[C@H](Cc2c[nH]c3ncccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0005989 (265) | 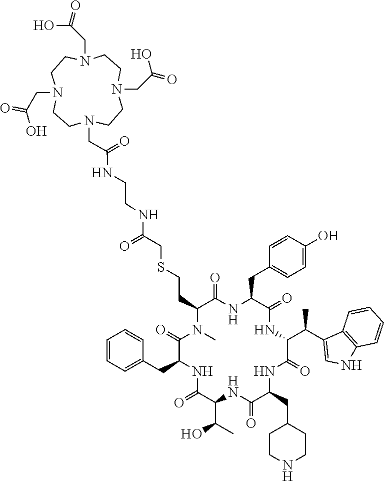 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCSCC(=O)NCCNC(=O)CN2CCN(CC(=O)O)CCN(CC(=O)O)CCN(CC(=O)O)CC2)N(C)C(=O)[C@H](Cc2ccccc2)NC1=O] |
| RB-0005991 (266) | 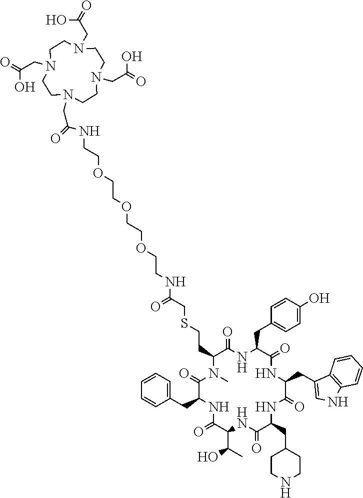 [SMILES: C[C@@H](O)[C@@H]1NC(=O)[C@H](CC2CCNCC2)NC(=O)[C@H](Cc2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0005993 (267) | 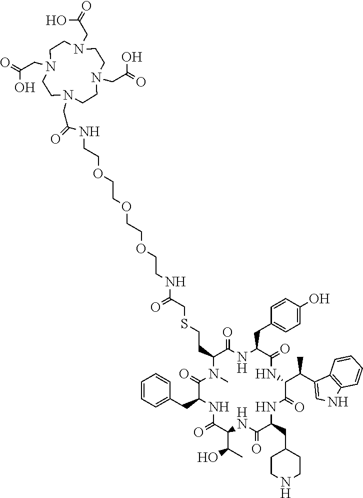 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCSCC(=O)NCCOCCOCCOCCNC(=O)CN2CCN(CC(=O)O)CCN(CC(=O)O)CCN(CC(=O)O)CC2)N(C)C(=O)[C@H](Cc2ccccc2)NC1=O] |
| RB-0006008 (268) | 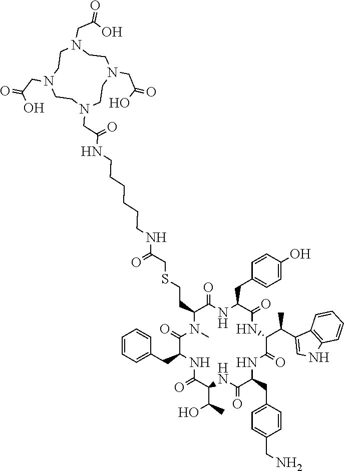 [SMILES: C[C@@H](O)[C@@H]1NC(=O)[C@H](Cc2ccc(CN)cc2)NC(=O)[C@@H]([C@@H](C)c2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006010 (269) | 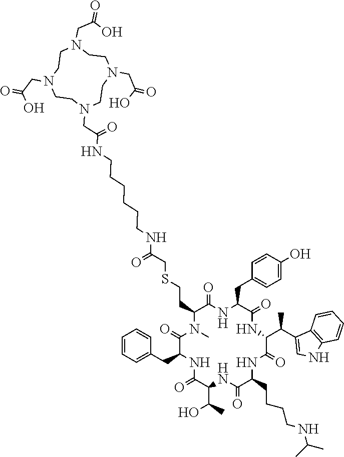 [SMILES: CC(C)NCCCC[C@@H]1NC(=O)[C@@H]([C@@H](C)c2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC(=O)[C@H]([C@@H](C)O)NC1=O] |
| RB-0006012 (270) | 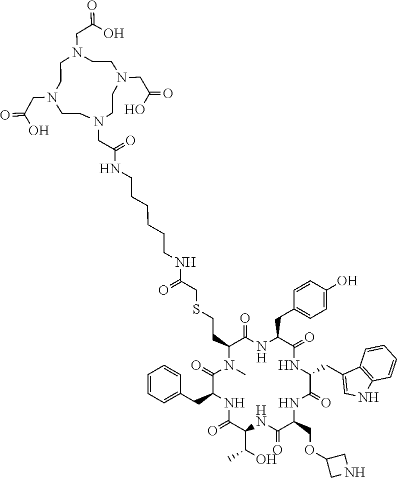 [SMILES: C[C@@H](O)[C@@H]1NC(=O)[C@H](COC2CNC2)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006014 (271) | 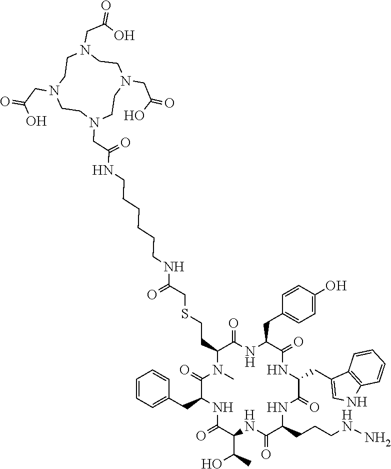 [SMILES: C[C@@H](O)[C@@H]1NC(=O)[C@H](CCCNN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006018 (272) | 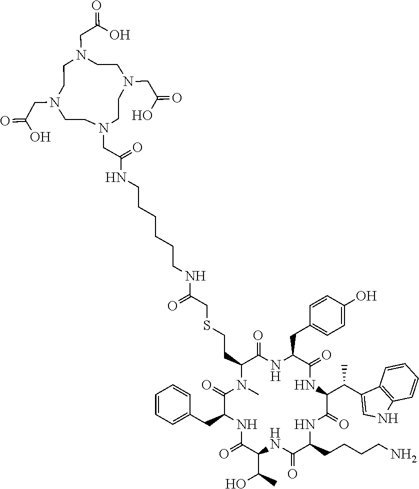 [SMILES: C[C@H](c1c[nH]c2ccccc12)[C@@H]1NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC(=O)[C@H]([C@@H](C)O)NC(=O)[C@H](CCCCN)NC1=O] |
| RB-0006058 (274) | 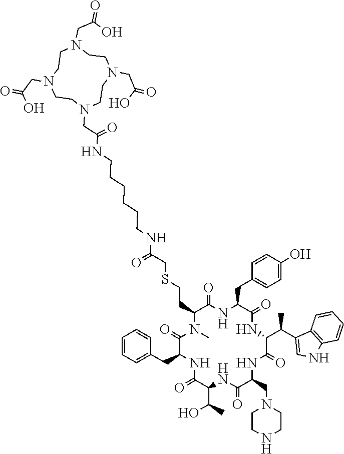 [SMILES: C[C@@H](O)[C@@H]1NC(=O)[C@H](CN2CCNCC2)NC(=O)[C@@H]([C@@H](C)c2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006063 (277) | 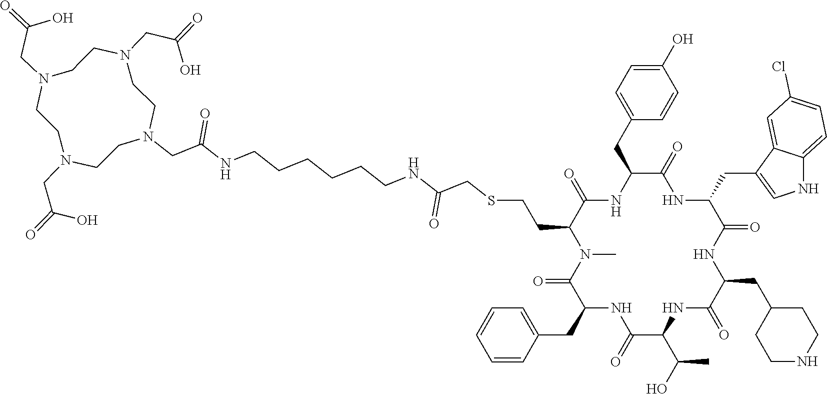 [SMILES: C[C@@H](O)[C@@H]1NC(=O)[C@H](CC2CCNCC2)NC(=O)[C@@H](Cc2c[nH]c3ccc(Cl)cc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006068 (273) | 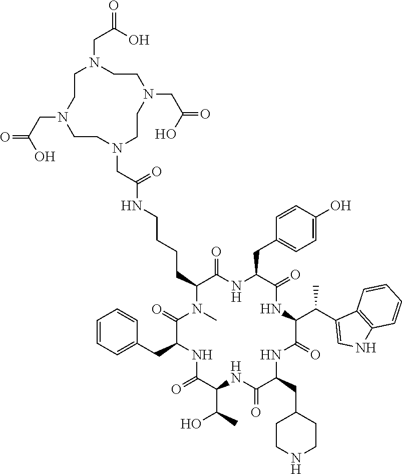 [SMILES: C[C@H](c1c[nH]c2ccccc12)[C@@H]1NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC(=O)[C@H]([C@@H](C)O)NC(=O)[C@H](CC2CCNCC2)NC1=O] |
| RB-0006094 (279) | 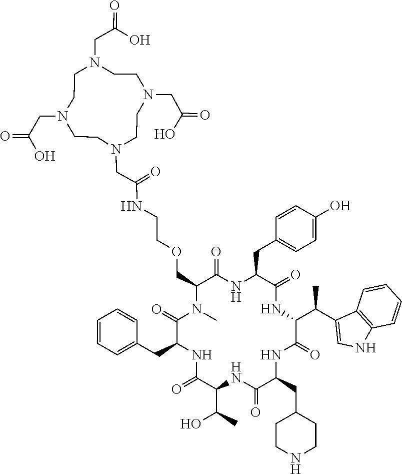 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](COCCNC(=O)CN2CCN(CC(=O)O)CCN(CC(=O)O)CCN(CC(=O)O)CC2)N(C)C(=O)[C@H](Cc2ccccc2)NC1=O] |
| RB-0006127 (280) | 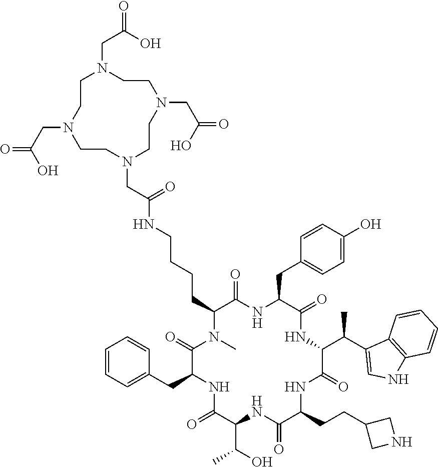 [SMILES: C[C@@H](O)[C@@H]1NC(=O)[C@H](CCC2CNC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006138 (281) | 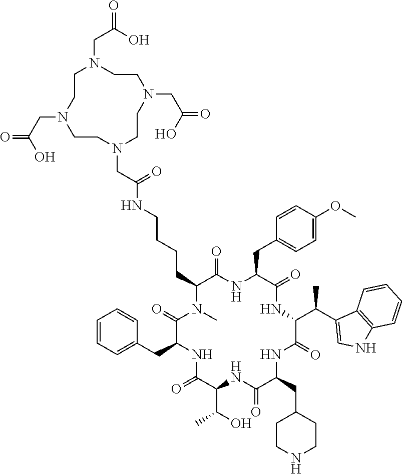 [SMILES: COc1ccc(C[C@@H]2NC(=O)[C@H](CCCCNC(=O)CN3CCN(CC(=O)O)CCN(CC(=O)O)CCN(CC(=O)O)CC3)N(C)C(=O)[C@H](Cc3ccccc3)NC(=O)[C@H]([C@@H](C)O)NC(=O)[C@H](CC3CCNCC3)NC(=O)[C@@H]([C@@H](C)c3c[nH]c4ccccc34)NC2=O)cc1] |
| RB-0006157 (282) | 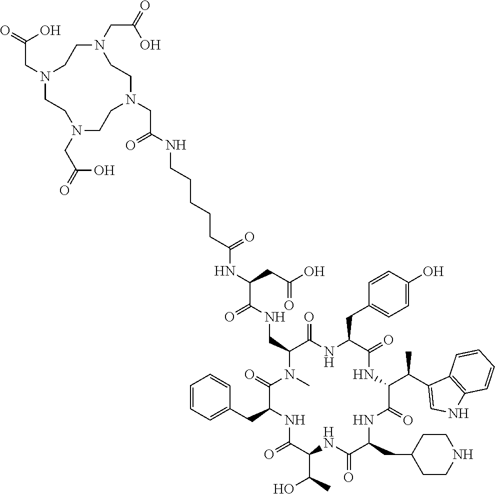 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CNC(=O)[C@H](CC(=O)O)NC(=O)CCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006159 (283) | 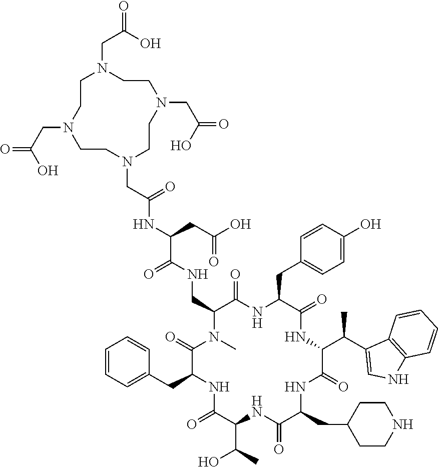 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CNC(=O)[C@H](CC(=O)O)NC(=O)CN2CCN(CC(=O)O)CCN(CC(=O)O)CCN(CC(=O)O)CC2)N(C)C(=O)[C@H](Cc2ccccc2)NC1=O] |
| RB-0006161 (284) | 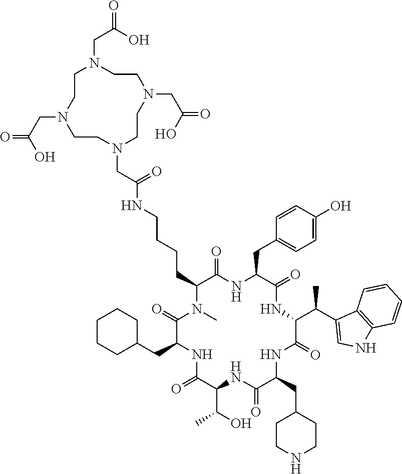 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](CC2CCCCC2)NC1=O] |
| RB-0006165 (285) | 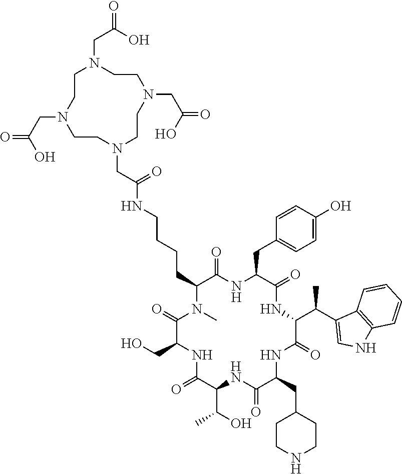 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](CO)NC1=O] |
| RB-0006169 (286) | 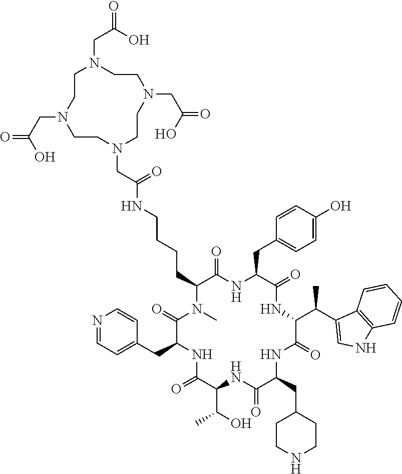 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccncc2)NC1=O] |
| RB-0006173 (287) | 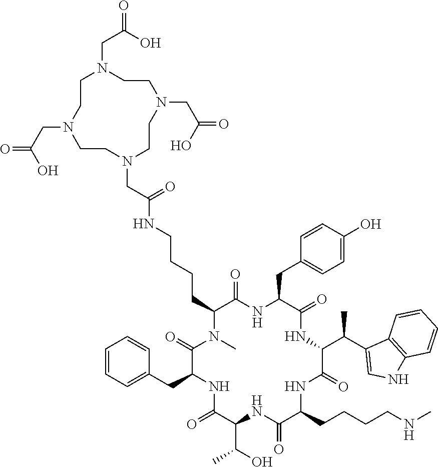 [SMILES: CNCCCC[C@@H]1NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC(=O)[C@H]([C@@H](C)O)NC1=O] |
| RB-0006175 (288) | 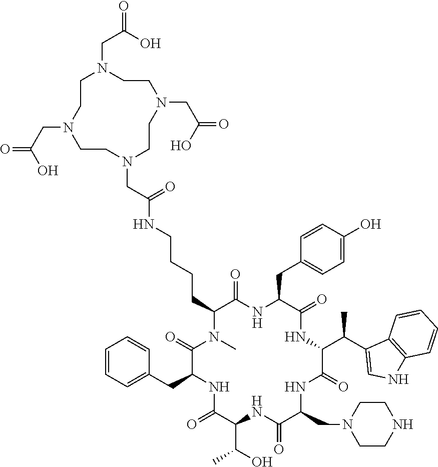 [SMILES: C[C@@H](O)[C@@H]1NC(=O)[C@H](CN2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006208 (290) | 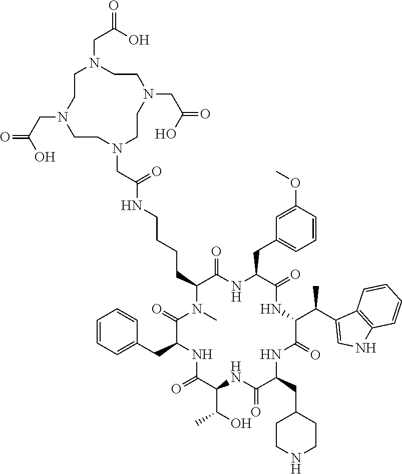 [SMILES: COc1cccc(C[C@@H]2NC(=O)[C@H](CCCCNC(=O)CN3CCN(CC(=O)O)CCN(CC(=O)O)CCN(CC(=O)O)CC3)N(C)C(=O)[C@H](Cc3ccccc3)NC(=O)[C@H]([C@@H](C)O)NC(=O)[C@H](CC3CCNCC3)NC(=O)[C@@H]([C@@H](C)c3c[nH]c4ccccc34)NC2=O)c1] |
| RB-0006212 (291) | 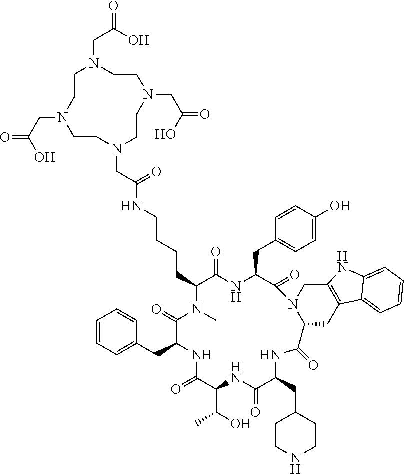 [SMILES: C[C@@H](O)[C@@H]1NC(=O)[C@H](CC2CCNCC2)NC(=O)[C@H]2Cc3c([nH]c4ccccc34)CN2C(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006214 (292) | 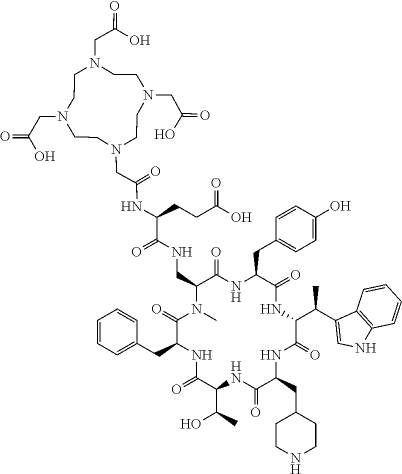 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CNC(=O)[C@H](CCC(=O)O)NC(=O)CN2CCN(CC(=O)O)CCN(CC(=O)O)CCN(CC(=O)O)CC2)N(C)C(=O)[C@H](Cc2ccccc2)NC1=O] |
| RB-0006216 (293) | 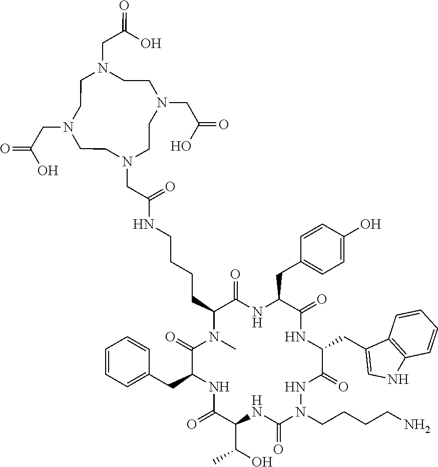 [SMILES: C[C@@H](O)[C@@H]1NC(=O)N(CCCCN)NC(=O)[C@@H](Cc2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006236 (295) | 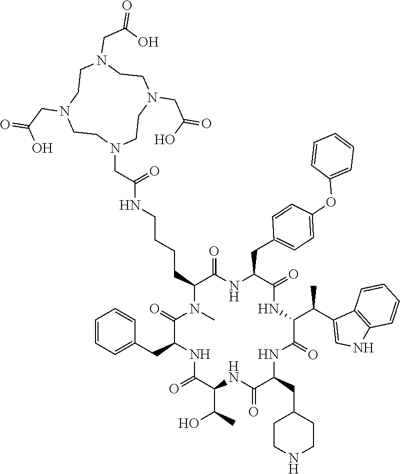 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(Oc3ccccc3)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006238 (296) | 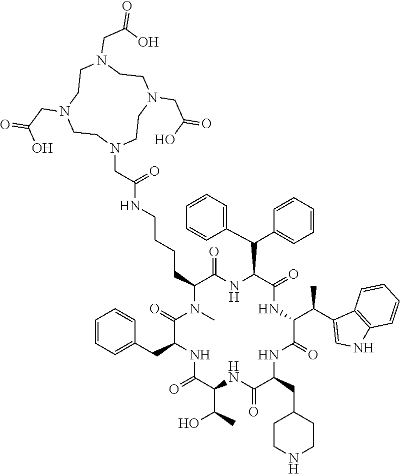 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](C(c2ccccc2)c2ccccc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006283 (298) | 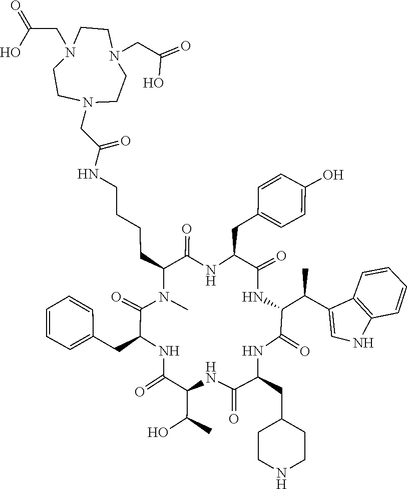 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CC2)N(C)C(=O)[C@H](Cc2ccccc2)NC1=O] |
| RB-0006284 (297) | 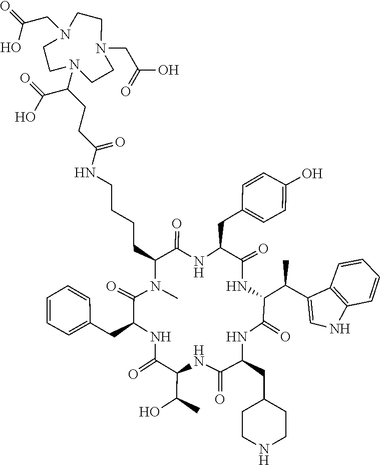 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CCC(C(=O)O)N2CCN(CC(=O)O)CCN(CC(=O)O)CC2)N(C)C(=O)[C@H](Cc2ccccc2)NC1=O] |
| RB-0006316 (299) | 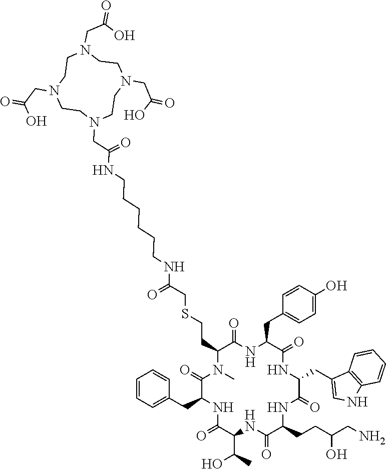 [SMILES: C[C@@H](O)[C@@H]1NC(=O)[C@H](CCC(O)CN)NC(=O)[C@@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006318 (300) | 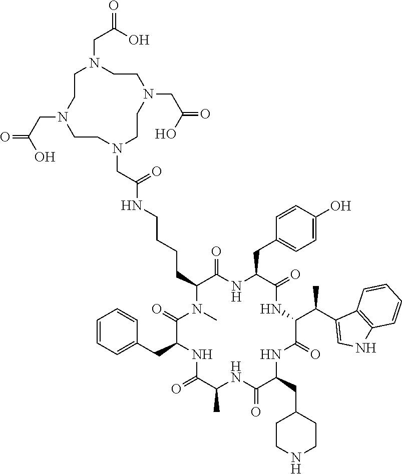 [SMILES: C[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006358 (311) | 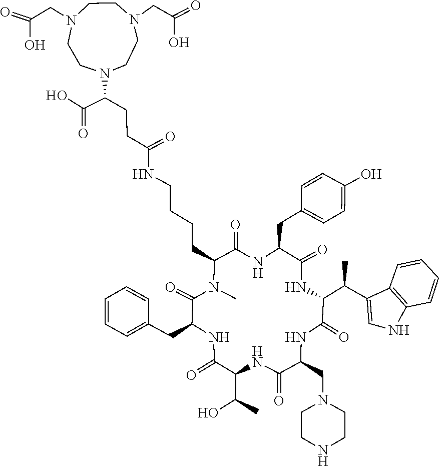 [SMILES: C[C@@H](O)[C@@H]1NC(=O)[C@H](CN2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CC[C@H](C(=O)O)N2CCN(CC(=O)O)CCN(CC(=O)O)CC2)N(C)C(=O)[C@H](Cc2ccccc2)NC1=O] |
| RB-0006384 (301) | 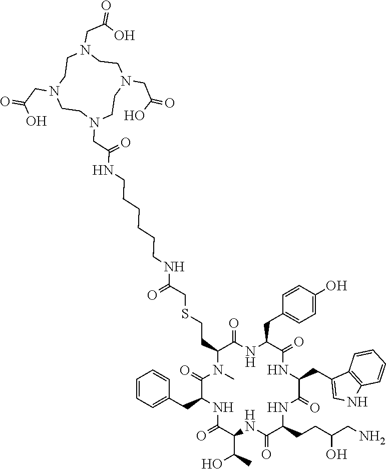 [SMILES: C[C@@H](O)[C@@H]1NC(=O)[C@H](CCC(O)CN)NC(=O)[C@H](Cc2c[nH]c3ccccc23)NC(=O)[C@H](Cc2ccc(O)cc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006386 (302) | 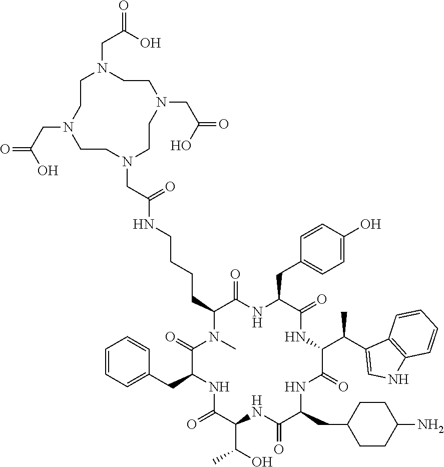 [SMILES: C[C@@H](O)[C@@H]1NC(=O)[C@H](CC2CCC(N)CC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006388 (303) | 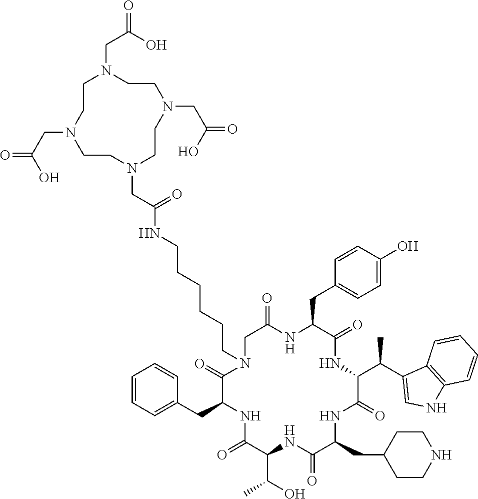 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)CN(CCCCCCNC(=O)CN2CCN(CC(=O)O)CCN(CC(=O)O)CCN(CC(=O)O)CC2)C(=O)[C@H](Cc2ccccc2)NC1=O] |
| RB-0006390 (304) | 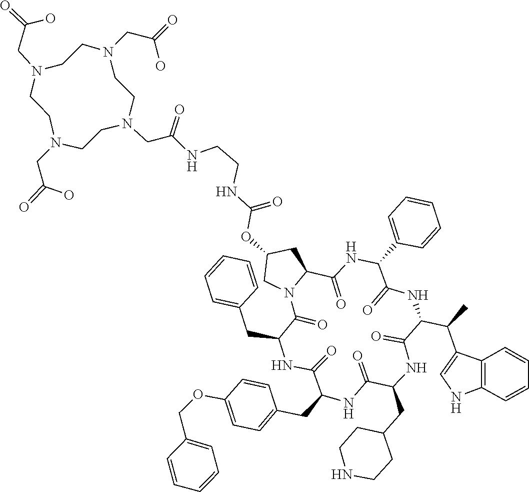 [SMILES: C[C@@H](c1c[nH]c2ccccc12)[C@H]1NC(=O)[C@@H](c2ccccc2)NC(=O)[C@@H]2C[C@@H](OC(=O)NCCNC(=O)CN3CCN(CC(=O)O)CCN(CC(=O)O)CCN(CC(=O)O)CC3)CN2C(=O)[C@H](Cc2ccccc2)NC(=O)[C@H](Cc2ccc(OCc3ccccc3)cc2)NC(=O)[C@H](CC2CCNCC2)NC1=O] |
| RB-0006392 (305) | 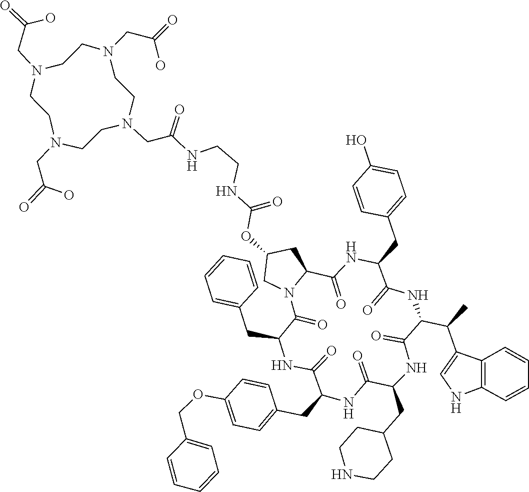 [SMILES: C[C@@H](c1c[nH]c2ccccc12)[C@H]1NC(=O)[C@H](Cc2ccc(O)cc2)NC(=O)[C@@H]2C[C@@H](OC(=O)NCCNC(=O)CN3CCN(CC(=O)O)CCN(CC(=O)O)CCN(CC(=O)O)CC3)CN2C(=O)[C@H](Cc2ccccc2)NC(=O)[C@H](Cc2ccc(OCc3ccccc3)cc2)NC(=O)[C@H](CC2CCNCC2)NC1=O] |
| RB-0006394 (306) | 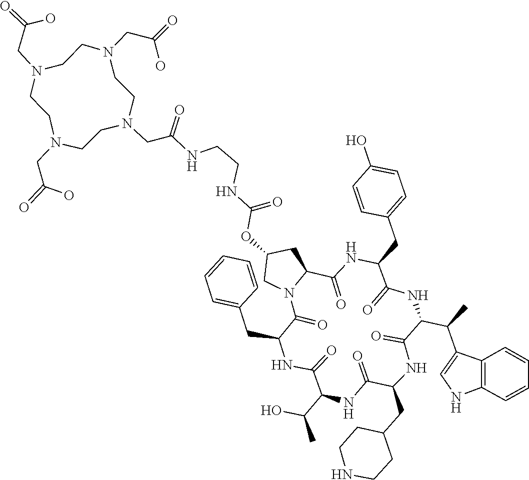 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@@H]2C[C@@H](OC(=O)NCCNC(=O)CN3CCN(CC(=O)O)CCN(CC(=O)O)CCN(CC(=O)O)CC3)CN2C(=O)[C@H](Cc2ccccc2)NC1=O] |
| RB-0006396 (307) | 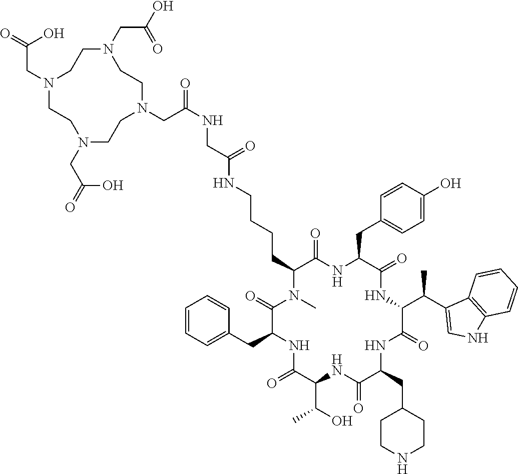 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CNC(=O)CN2CCN(CC(=O)O)CCN(CC(=O)O)CCN(CC(=O)O)CC2)N(C)C(=O)[C@H](Cc2ccccc2)NC1=O] |
| RB-0006399 (308) | 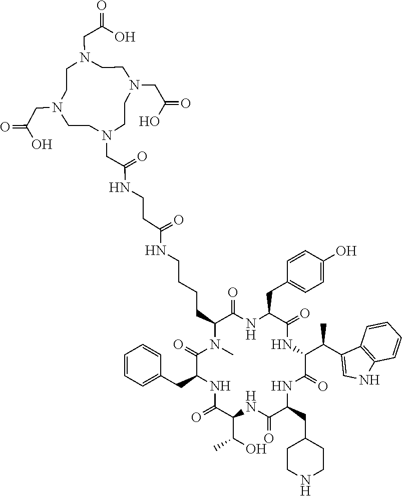 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CCNC(=O)CN2CCN(CC(=O)O)CCN(CC(=O)O)CCN(CC(=O)O)CC2)N(C)C(=O)[C@H](Cc2ccccc2)NC1=O] |
| RB-0006402 (309) | 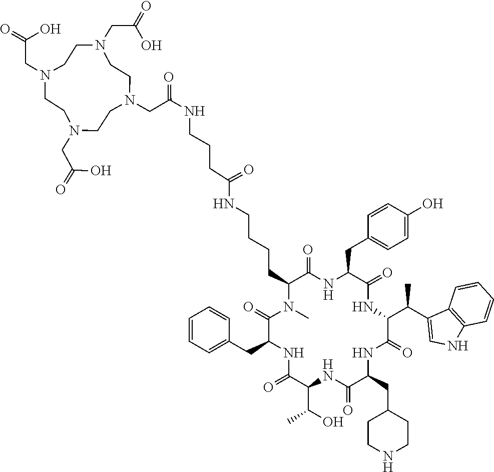 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CCCNC(=O)CN2CCN(CC(=O)O)CCN(CC(=O)O)CCN(CC(=O)O)CC2)N(C)C(=O)[C@H](Cc2ccccc2)NC1=O] |
| RB-0006405 (310) | 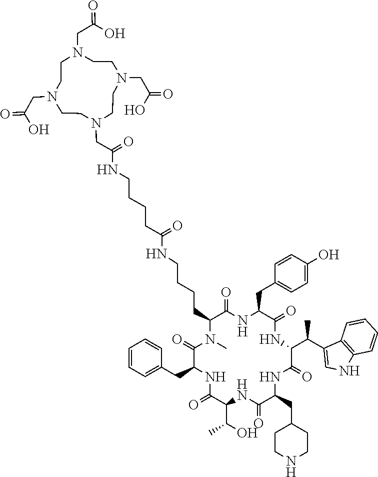 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006416 (312) | 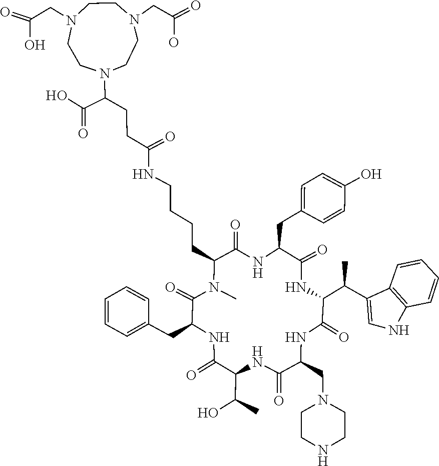 [SMILES: C[C@@H](O)[C@@H]1NC(=O)[C@H](CN2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CCC(C(=O)O)N2CCN(CC(=O)O)CCN(CC(=O)O)CC2)N(C)C(=O)[C@H](Cc2ccccc2)NC1=O] |
| RB-0006418 (313) | 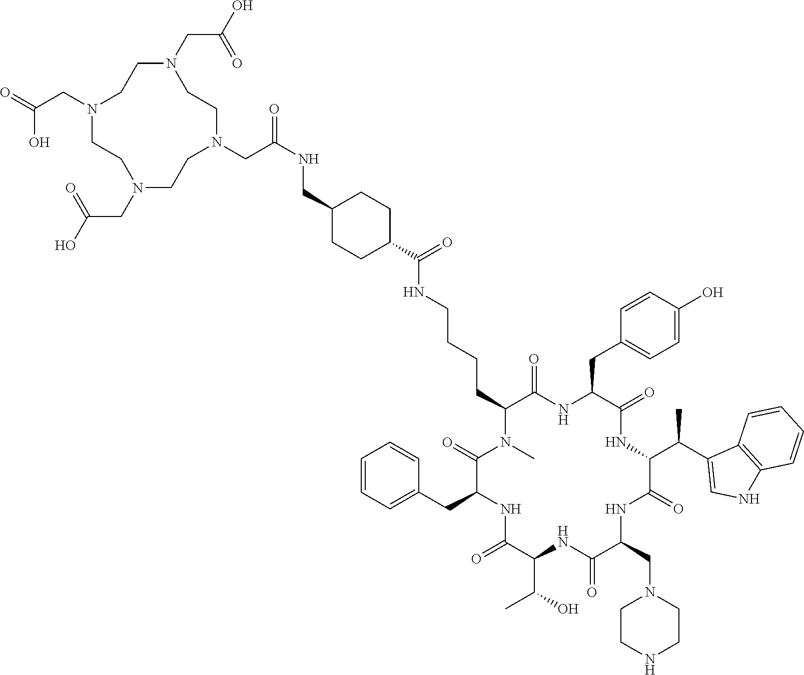 [SMILES: C[C@@H](O)[C@@H]1NC(=O)[C@H](CN2CCNCC2)NC(=O)[C@@H]([C@@H](C)c2c[nH]c3ccccc23)NC(=O)[C@H](Cc2ccc(O)cc2)NC(=O)[C@H](CCCCNC(=O)[C@H]2CC[C@H](CNC(=O)CN3CCN(CC(=O)O)CCN(CC(=O)O)CCN(CC(=O)O)CC3)CC2)N(C)C(=O)[C@H](Cc2ccccc2)NC1=O] |
| RB-0006448 (314) | 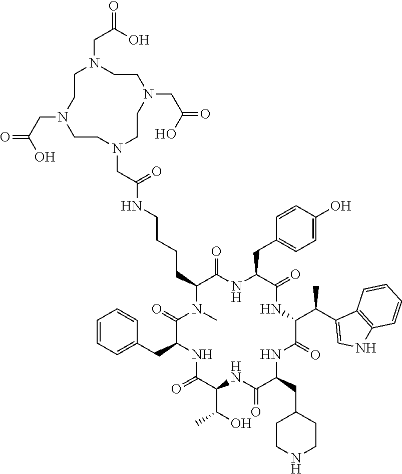 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006451 (315) | 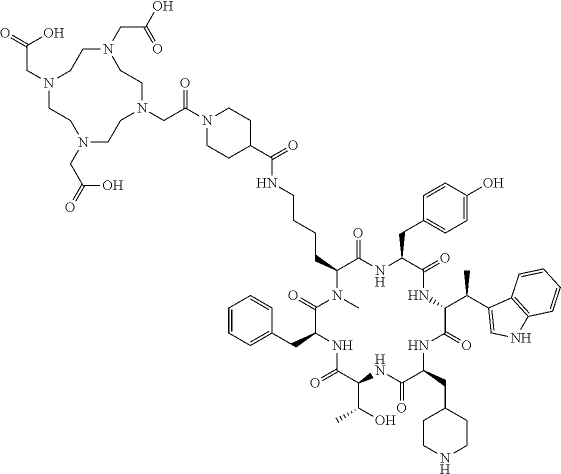 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)C2CCN(C(=O)CN3CCN(CC(=O)O)CCN(CC(=O)O)CCN(CC(=O)O)CC3)CC2)N(C)C(=O)[C@H](Cc2ccccc2)NC1=O] |
| RB-0006454 (316) | 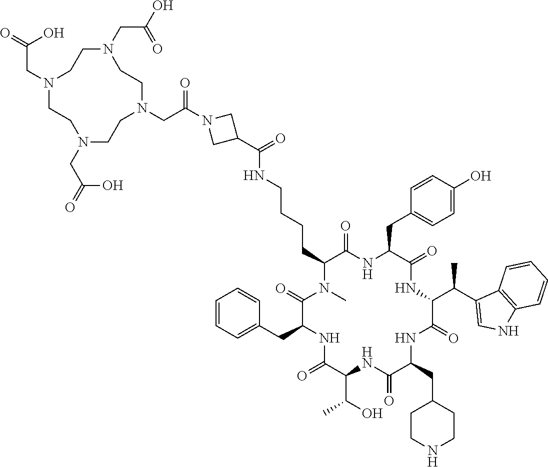 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)C2CN(C(=O)CN3CCN(CC(=O)O)CCN(CC(=O)O)CCN(CC(=O)O)CC3)C2)N(C)C(=O)[C@H](Cc2ccccc2)NC1=O] |
| RB-0006457 (317) | 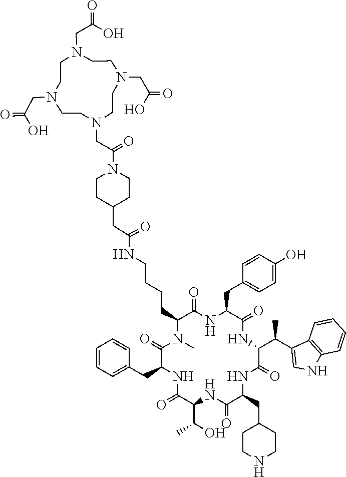 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CC2CCN(C(=O)CN3CCN(CC(=O)O)CCN(CC(=O)O)CCN(CC(=O)O)CC3)CC2)N(C)C(=O)[C@H](Cc2ccccc2)NC1=O] |
| RB-0006460 (318) | 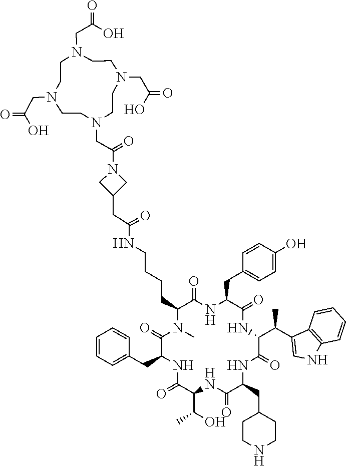 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CC2CN(C(=O)CN3CCN(CC(=O)O)CCN(CC(=O)O)CCN(CC(=O)O)CC3)C2)N(C)C(=O)[C@H](Cc2ccccc2)NC1=O] |
| RB-0006463 (319) | 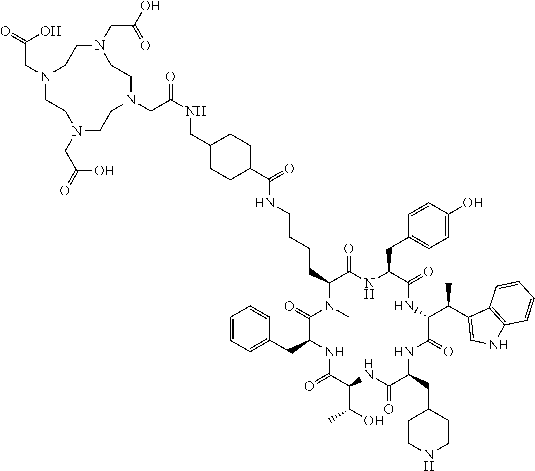 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)C2CCC(CNC(=O)CN3CCN(CC(=O)O)CCN(CC(=O)O)CCN(CC(=O)O)CC3)CC2)N(C)C(=O)[C@H](Cc2ccccc2)NC1=O] |
| RB-0006469 (320) | 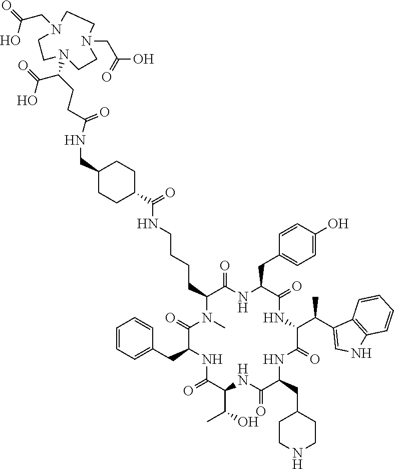 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)[C@H]2CC[C@H](CNC(=O)CC[C@H](C(=O)O)N3CCN(CC(=O)O)CCN(CC(=O)O)CC3)CC2)N(C)C(=O)[C@H](Cc2ccccc2)NC1=O] |
| RB-0006471 (319) | 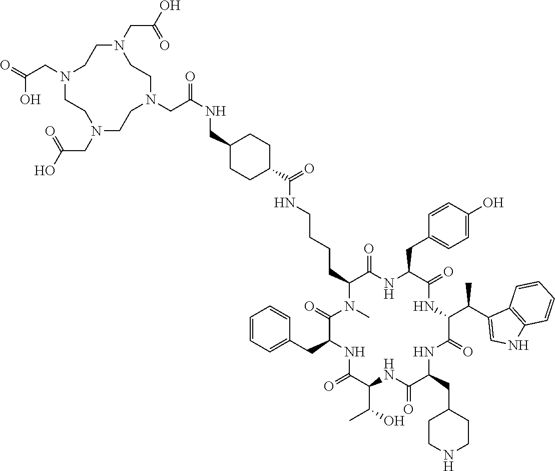 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)[C@H]2CC[C@H](CNC(=O)CN3CCN(CC(=O)O)CCN(CC(=O)O)CCN(CC(=O)O)CC3)CC2)N(C)C(=O)[C@H](Cc2ccccc2)NC1=O] |
| RB-0006475 (321) | 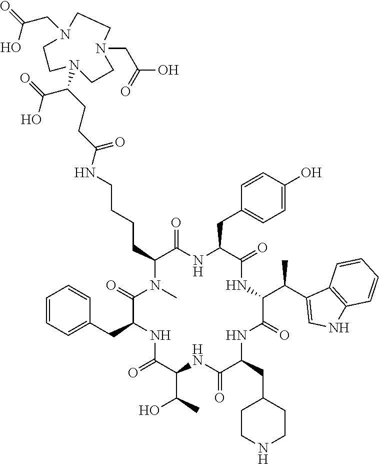 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CC[C@H](C(=O)O)N2CCN(CC(=O)O)CCN(CC(=O)O)CC2)N(C)C(=O)[C@H](Cc2ccccc2)NC1=O] |
| RB-0006487 (322) | 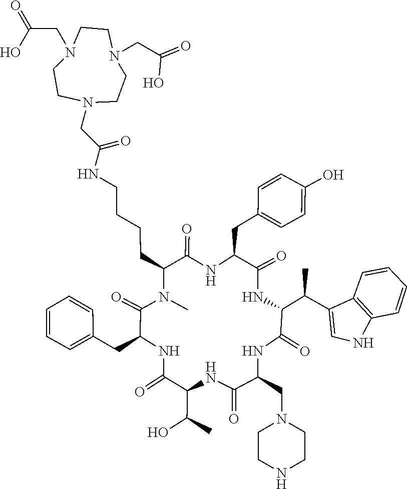 [SMILES: C[C@@H](O)[C@@H]1NC(=O)[C@H](CN2CCNCC2)NC(=O)[C@@H]([C@@H](C)c2c[nH]c3ccccc23)NC(=O)[C@H](Cc2ccc(O)cc2)NC(=O)[C@H](CCCCNC(=O)CN2CCN(CC(=O)O)CCN(CC(=O)O)CC2)N(C)C(=O)[C@H](Cc2ccccc2)NC1=O] |
| RB-0006534 (323) | 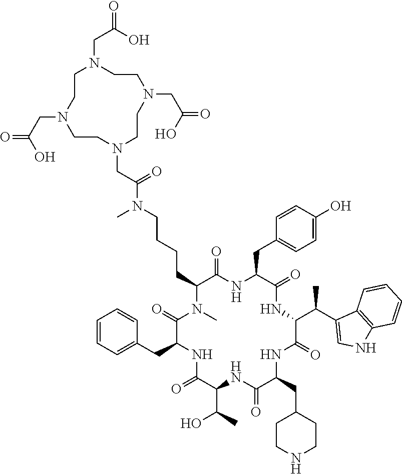 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCN(C)C(=O)CN2CCN(CC(=O)O)CCN(CC(=O)O)CCN(CC(=O)O)CC2)N(C)C(=O)[C@H](Cc2ccccc2)NC1=O] |
| RB-0006552 (323) | 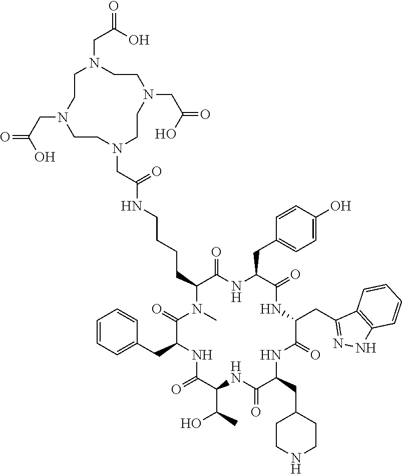 [SMILES: C[C@@H](O)[C@@H]1NC(=O)[C@H](CC2CCNCC2)NC(=O)[C@@H](Cc2n[nH]c3ccccc23)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)N(C)C(=O)[C@H](Cc2ccccc2)NC1=O] |
| RB-0006557 (324) | 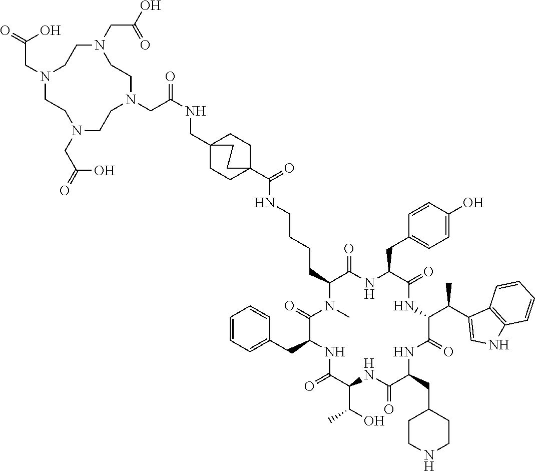 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCNC(=O)C23CCC(CNC(=O)CN4CCN(CC(=O)O)CCN(CC(=O)O)CCN(CC(=O)O)CC4)(CC2)CC3)N(C)C(=O)[C@H](Cc2ccccc2)NC1=O] |
| RB-0006580 (326) | 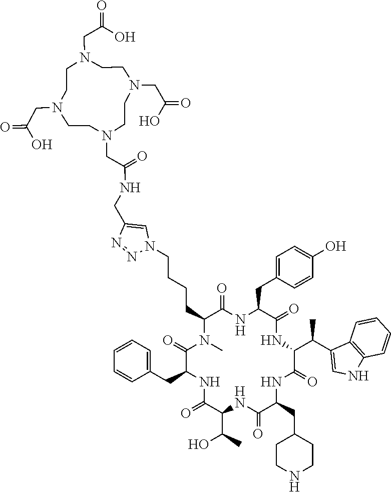 [SMILES: C[C@@H](O)[C@@H]1NC(=O)[C@H](CC2CCNCC2)NC(=O)[C@@H]([C@@H](C)c2c[nH]c3ccccc23)NC(=O)[C@H](Cc2ccc(O)cc2)NC(=O)[C@H](CCCCn2cc(CNC(=O)CN3CCN(CC(=O)O)CCN(CC(=O)O)CCN(CC(=O)O)CC3)nn2)N(C)C(=O)[C@H](Cc2ccccc2)NC1=O] |

In one aspect, provided herein is a compound of Table 2, or a pharmaceutically acceptable salt thereof. In one aspect, provided herein is a compound of Table 3, or a pharmaceutically acceptable salt thereof.

In one aspect, provided herein is a conjugate of the following structural formula:

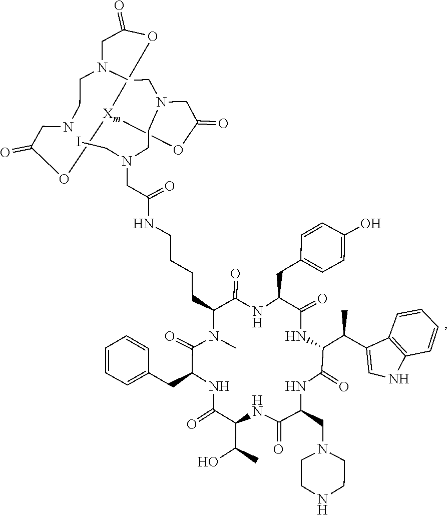

or a pharmaceutically acceptable salt thereof, wherein Xₘ is a radionuclide.

In another aspect, provided herein is a conjugate of the following structural formula:

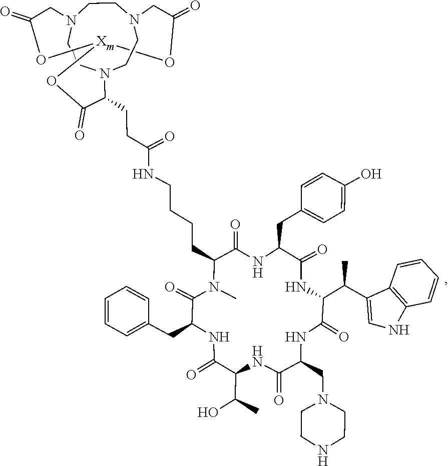

or a pharmaceutically acceptable salt thereof, wherein Xₘ is a radionuclide.

In another aspect, provided herein is a conjugate of the following structural formula:

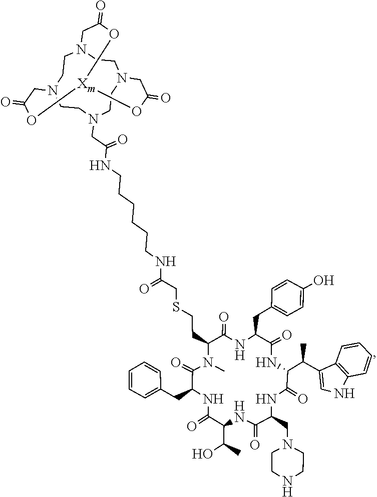

or a pharmaceutically acceptable salt thereof, wherein Xₘ is a radionuclide.

In another aspect, provided herein is a conjugate of the following structural formula:

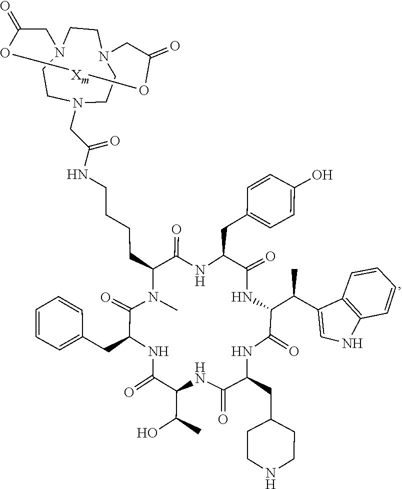

or a pharmaceutically acceptable salt thereof, wherein Xₘ is a radionuclide.

In another aspect, provided herein is a conjugate of the following structural formula:

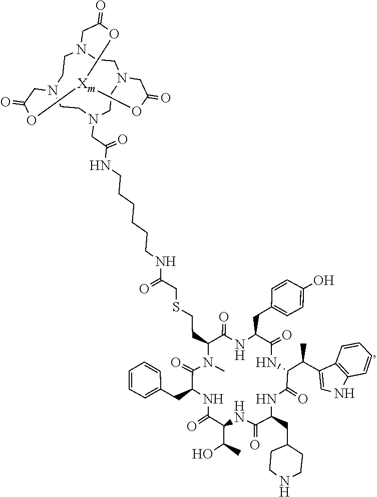

or a pharmaceutically acceptable salt thereof, wherein Xₘ is a radionuclide.

In another aspect, provided herein is a conjugate of the following structural formula:

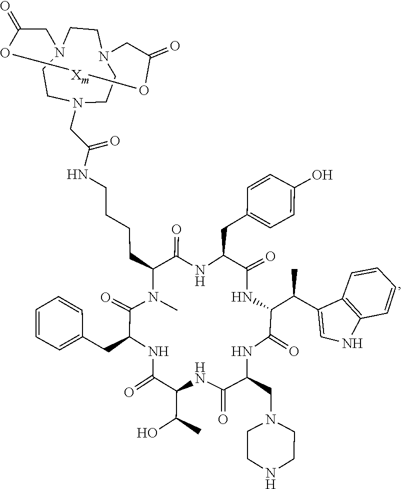

or a pharmaceutically acceptable salt thereof, wherein Xₘ is a radionuclide.

In another aspect, provided herein is a conjugate of the following structural formula:

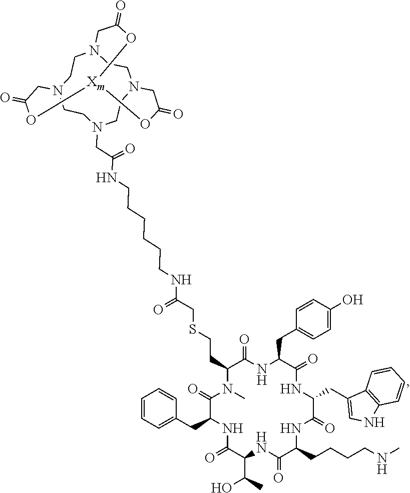

or a pharmaceutically acceptable salt thereof, wherein Xₘ is a radionuclide.

### Linker

A conjugate described herein can comprise one or more linkers. In some embodiments, the linker covalently attaches the peptide with the metal chelator. In some embodiments, the peptide attaches directly to the metal chelator without a linker.

In some embodiments, the present disclosure describes linkers that function as a spacer. A linker can comprise a number of intervening atoms (on a linear chain, excluding pendant groups or substituents) between the metal chelator and the binding peptide thereby creating a distance between the metal chelator and the binding peptide. In some embodiments, a linker comprises 10-100 intervening atoms between the metal chelator and the binding peptide. In some embodiments, a linker comprises 2-60 intervening atoms between the metal chelator and the binding peptide. In some embodiments, a linker comprises 2 to 20, 2 to 50, 5 to 15, 5 to 25, 10 to 40, 30 to 60, or 10 to 20 intervening atoms between the metal chelator and the binding peptide. In some embodiments, a linker comprises 3 to 30 intervening atoms between the metal chelator and the binding peptide. In some embodiments, a linker comprises 5 to 25 intervening atoms between the metal chelator and the binding peptide. In some embodiments, a linker comprises 6 to 18 intervening atoms between the metal chelator and the binding peptide. In some embodiments, a linker comprises 10 to 20 intervening atoms between the metal chelator and the binding peptide. The intervening atoms can comprise 1 or more carbons, and optionally one or more heteroatoms such as O and N. In some embodiments, the intervening atoms comprise 2 to 20, 2 to 50, 5 to 15, 5 to 25, 10 to 40, 30 to 60, or 10 to 20 carbons. In some embodiments, the intervening atoms comprise 0, 1, 2, 3, 4, 5, or 6 nitrogen atoms. In some embodiments, the intervening atoms comprise 0, 1, 2, 3, 4, 5, 6, 7 or 8 oxygen atoms. In some embodiments, the intervening atoms comprise 1 to 6 nitrogen and 0 to 4 oxygen atoms.

A linker can comprise one or more amino acid residues. In some embodiments, the linker comprises 1 to 3, 1 to 5, 1 to 10, 5 to 10, or 5 to 20 amino acid residues. In some embodiments, the linker comprises 1, 2, 3, 4, 5, 6, 7, 8, 9, or 10 amino acid residues. In some embodiments, the linker comprises 1 to 5 amino acid residues. For example, the linker can comprise one or more lysine (K) residues such as K, KK, or KKK sequences. In some embodiments, the linker comprises an ornithine, a lysine, a homolysine, an aspartate, a beta-aspartate, a glutamate, a 2-aminosuberic acid, a glycine, a beta-alanine, or a combination thereof. In some embodiments, the linker comprises a lysine, an aspartate, a beta-aspartate, a glutamate, or a derivative thereof. In some embodiments, the linker comprises an ornithine, a lysine, or a homolysine. In some embodiments, the linker comprises an aspartic acid, a beta-aspartate, a glutamic acid, or a 2-aminosuberic acid. In some embodiments, the linker comprises a glycine or a beta-alanine.

A herein-described linker can attach to X1 as shown in Formula (IV1), X2 as shown in Formula (IV2), X3 as shown in formula (IV3), X4 as shown in Formula (IV4), X5 as shown in Formula (IV5), or X6 as shown in Formula (IV6). The linker can be bonded to the peptide, the metal chelator, or both, for example, through a chemically reactive group. Exemplary chemically reactive groups include, but are not limited to, a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cysteinyl residues). The site to which the linker is bound to the peptide can be a natural or unnatural amino acid of the peptide and/or it can be introduced into the peptide, e.g., by DNA recombinant technology (e.g., by introducing a cysteine or protease cleavage site in the amino acid sequence) or by protein biochemistry (e.g., reduction, pH adjustment or proteolysis). Exemplary methods for attaching the linker includes carbodiimide reaction, reactions using bifunctional agents such as dialdehydes or imidoesters, Schiff base reaction, Suzuki-Miyaura cross-coupling reactions, Isothiocyanates as coupling agents, and click chemistry.

The linker can have a prescribed length thereby linking the metal chelator (and optionally radionuclide) and the peptide while allowing an appropriate distance therebetween. In some embodiments, the linker has 1 to 100 atoms, 1 to 60 atoms, 1 to 30 atoms, 1 to 15 atoms, 1 to 10 atoms, 1 to 5, or 2 to 20 atoms in length. In some embodiments, the linker has 1 to 10 atoms in length.

The linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 327)), proline-rich sequences, and regions rich in double and/or triple bonds.

In some embodiments, a linker may be further added to the (cyclic) peptide. Examples of the linker include the foregoing amino acid linker (peptide linker), a chemical linker, a fatty acid linker, a nucleic acid linker, a sugar chain linker, or the like, or it may be a complex, for example, a chemical linker, a peptide linker, or the like. Examples of the chemical linker include a PEG (polyethylene glycol) linker. For example, the PEG linker may comprise between 1 to 24 ethylene glycol units. Furthermore, the linker may be a fatty acid linker containing a divalent chemical moiety derived from a fatty acid. In some embodiments, the linker comprises at least one amino acid, and, for example, a glycine-rich peptide such as a peptide having a sequence [Gly-Gly-Gly-Gly-Ser]ₙ (in the formula, n is 1, 2, 3, 4, 5, or 6) (SEQ ID NO: 328).

The linker can be cleavable, e.g., under physiological conditions, e.g., under intracellular conditions, such that cleavage of the linker releases the chelator and radionuclide in the intracellular environment. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker.

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, a linker of the present disclosure is a bond. In some embodiments, a linker of the present disclosure comprises a substituted or unsubstituted C₁₋₁₂heteroalkylene. In some embodiments, a linker of the present disclosure comprises a substituted or unsubstituted C₁₋₈heteroalkylene. In some embodiments, a linker of the present disclosure comprises a substituted or unsubstituted C₁₋₄heteroalkylene. In some embodiments, the heteroalkylene is substituted with one or more R^{e}, wherein each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl. In some embodiments, the heteroalkylene is substituted with one or more R^{e}, wherein each R^{e} is independently —OH, oxo, —O—C₁-C₆alkyl, —NH₂, —NHC₁-C₆alkyl, —N(C₁-C₆alkyl)₂, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, or —C(═O)NHC₁-C₆alkyl.

In some embodiments, the linker comprises polyethylene glycol such as (—CH₂—CH₂—O—)₁₋₁₀.

In some embodiments, the linker comprises a conjugation moiety such as a click chemistry residue. In some embodiments, the conjugation moiety is a reaction product of a conjugation group. In some embodiments, the linker is attached to the peptide, to the metal chelator, or both via click chemistry, thereby forming a click chemistry residue. For example, the peptide can comprise an azide group (at N- or C-terminus or at a non-terminal amino acid) that reacts with an alkyne group to form the linker of a conjugate. For another example, the peptide can comprise an alkyne group (at N- or C-terminus or at a non-terminal amino acid) that reacts with an azide to form the linker of a conjugate. The metal chelator and the linker can be attached similarly. In some embodiments, the linker comprises an azide moiety, an alkyne moiety, or both. In some embodiments, the linker comprises a triazole. In some embodiments, the linker comprises 1,4-di-substituted 1,2,3-triazole. In some embodiments, the click chemistry residue is

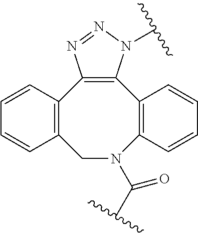 [SMILES: *C(=O)N1Cc2ccccc2-c2nnn(*)c2-c2ccccc21]

(DBCO-azide residue),

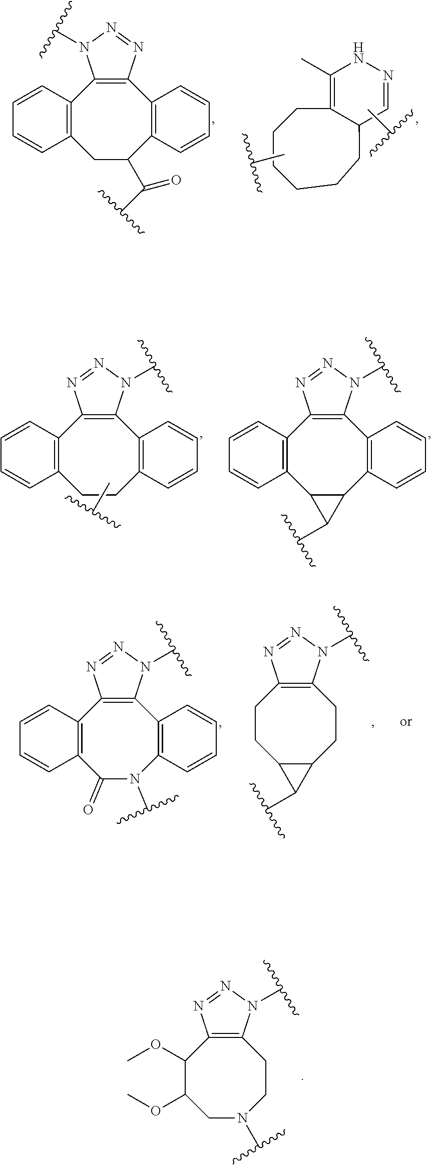

In some embodiments, the click chemistry residue is a DIBO-azide residue, BARAC-azide residue, DBCO-azide residue, DIFO-azide residue, COMBO-azide residue, BCN-azide residue, or DIMAC-azide residue. In some embodiments, the linker comprises a residue of nitrone dipole cycloaddition. In some embodiments, the linker comprises a residue of tetrazine ligation. In some embodiments, the linker comprises a residue of quadricyclane ligation. Exemplary groups of click chemistry residue are shown in Hein at al., “Click Chemistry, A Powerful Tool for Pharmaceutical Sciences,” Pharmaceutical Research volume 25, pages 2216-2230 (2008); Thirumurugan et al, “Click Chemistry for Drug Development and Diverse Chemical-Biology Applications,” Chem. Rev. 2013, 113, 7, 4905-4979; US20160107999A1; U.S. Pat. No. 10,266,502B2; and US20190204330A1, each of which is incorporated by reference in its entirety.

In some embodiments, the linker has a structure of

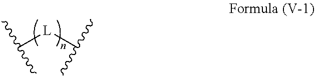

wherein each L is independently —O—, —NR^{L}—, —N(R^{L})₂⁺—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀ heteroalkylene, —(C₁-C₃₀ alkylene)-O—, —O—(C₁-C₃₀ alkylene)-, —(C₁-C₃₀ alkylene)-NR^{L}—, —NR^{L}—(C₁-C₃₀ alkylene)-, —(C₁-C₃₀ alkylene)-N(R^{L})₂⁺—, —N(R^{L})₂⁺—(C₁-C₃₀ alkylene)-, or a click chemistry residue; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20 (e.g., 1, 2, 3, 4, 5, 6, 7, 8, 9, 10, 11, 12, 13, 14, 15, 16, 17, 18, 19, or 20).

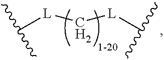

In some embodiments, the linker has a structure of wherein each L is independently —O—, —NR^{L}—, —N(R^{L})₂⁺—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—.

In some embodiments, the linker of Formula (V-1) has a structure of Formula (V-1a),

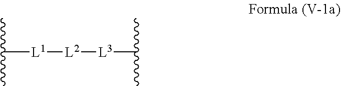

wherein
each of L¹ and L³ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)C₁-C₆alkylene-, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or —S(═O)₂NR^{L}C(═O)—; and
L² is absent, substituted or unsubstituted C₁-C₃₀ alkylene, or substituted or unsubstituted C₁-C₃₀ heteroalkylene.

In some embodiments, the linker comprises a structure of Formula (V1b),

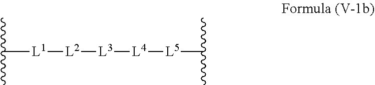

wherein each of L¹ and L⁵ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)C₁-C₆alkylene-, —C(═O)O—, —OC(═O)—, —OC(═O)O—C(═O)NR⁵—, —NR^{a}C(═O)—, —OC(═O)NR⁵—, —NR^{a}C(═O)O—, —NR^{a}C(═O)NR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₄-C₆ cycloalkyl, or substituted or unsubstituted 4- to 6-membered heterocycloalkyl; and
L², L³ and L⁴ are each independently absent, substituted or unsubstituted C₄-C₁₀cycloalkyl, substituted or unsubstituted 4- to 6-membered heterocycloalkyl, substituted or unsubstituted C₁-C₃₀ alkylene, or substituted or unsubstituted C₁-C₃₀ heteroalkylene.

In some embodiments, L¹ is —NH— or substituted or unsubstituted 4- to 6-membered heterocycloalkyl.

In some embodiments, L² is absent. In some embodiments, L² is substituted or unsubstituted C₁-C₃₀ alkylene, or substituted or unsubstituted C₁-C₃₀ heteroalkylene. In some embodiments, L² is substituted or unsubstituted C₁-C₃₀ alkylene. In some embodiments, L² is substituted or unsubstituted C₁-C₃₀ heteroalkylene. In some embodiments, L² is substituted or unsubstituted C₁-C₁₈ alkylene, or substituted or unsubstituted C₁-C₁₈ heteroalkylene. In some embodiments, L² is optionally substituted. In some embodiments, L² is optionally substituted with one or more substituents independently selected from —OH, —SH, oxo, amino, C₁-C₆ alkyl, C₁-C₆ hydroxyalkyl, C₁-C₆ haloalkyl, C₁-C₆ aminoalkyl, —C(═O)OR^{L}, —C₁-C₆alkylene-C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}. In some embodiments, L² is C₁-C₃₀ heteroalkylene that is optionally substituted with one or more substituents independently selected from —OH, —SH, oxo, amino, C₁-C₆ alkyl, C₁-C₆ hydroxyalkyl, C₁-C₆ haloalkyl, and C₁-C₆ aminoalkyl. In some embodiments, L² is optionally substituted with C₁-C₆ alkyl which is further optionally substituted with one or more substituents independently selected from —OH, —SH, oxo, amino, C₆-C₁₀ aryl, 6- to 10-membered heteroaryl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}.

In some embodiments, L³ is —NH—. In some embodiments, L³ is substituted or unsubstituted C₄-C₆ cycloalkyl or substituted or unsubstituted 4- to 6-membered heterocycloalkyl. In some embodiments, L³ is absent.

In some embodiments, L⁴ is absent. In some embodiments, L⁴ is substituted or unsubstituted C₄-C₆ cycloalkyl, substituted or unsubstituted 4- to 6-membered heterocycloalkyl, substituted or unsubstituted C₁-C₃₀ alkylene, or substituted or unsubstituted C₁-C₃₀ heteroalkylene.

In some embodiments, L⁵ is —NH—, —C(═O)—, or —C(═O)C₁-C₆alkylene-. In some embodiments, L⁵ is —NH—. In some embodiments, L⁵ is —C(═O)—. In some embodiments, L⁵ is —C(═O)C₁-C₆alkylene-.

In some embodiments for Formula (V-1b), L¹ is —NH— or substituted or unsubstituted 4- to 6-membered heterocycloalkyl; L⁵ is —NH—C(═O)—, or —C(═O)C₁-C₆alkylene-; L², L³ and L⁴ are each independently absent, substituted or unsubstituted C₄-C₁₀cycloalkyl, substituted or unsubstituted 4- to 6-membered heterocycloalkyl, substituted or unsubstituted C₁-C₁₂ alkylene, or substituted or unsubstituted C₁-C₃₀ heteroalkylene, wherein L¹ is connected to the metal chelator and L⁵ is connected to the peptide. In some embodiments, the cycloalkyl and heterocycloalkyl are unsubstituted and the alkylene and heteroalkylene are optionally substituted with 1 to 3 groups selected from the group consisting of oxo, —C(═O)OR^{L}, and —C₁-C₆alkylene-C(═O)OR^{L}.

In some embodiments for Formula (V-1b), L² is unsubstituted C₁-C₁₂ alkylene, and L³ and L⁴ are absent. In some embodiments for Formula (V-1b), L² is unsubstituted C₁-C₁₂ heteroalkylene, and L³ and L⁴ are absent.

In some embodiments, the linker comprises substituted or unsubstituted C₁-C₃₀ alkylene, C₁-C₁₂ alkylene, C₁-C₈ alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, the linker L (or L¹, L², L³, L⁴, or L⁵) is substituted with one or more R^{e}, wherein each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl. In some embodiments, each R^{e} is independently —OH, oxo, —O—C₁-C₆alkyl, —NH₂, —NHC₁-C₆alkyl, —N(C₁-C₆alkyl)₂, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, or —C(═O)NHC₁-C₆alkyl.

In some embodiments, each of L¹ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L¹ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L¹ is —O—, —NH—, —S(═O)—, —S(═O)₂—, or —C(═O)—. In some embodiments, L¹ is —C(═O)NH— or —NHC(═O)—. In some embodiments, L¹ is substituted or unsubstituted C₃-C₁₅ cycloalkyl, or substituted or unsubstituted C₁-C₁₂ heterocycloalkyl. In some embodiments, L¹ is substituted or unsubstituted aryl or substituted or unsubstituted heteroaryl. In some embodiments, L¹ is substituted or unsubstituted C₁-C₃₀ alkylene. In some embodiments, L¹ is substituted or unsubstituted C₂-C₃₀ alkenylene. In some embodiments, L¹ is substituted or unsubstituted C₁-C₃₀ heteroalkylene. In some embodiments, L¹ is substituted or unsubstituted C₅-C₂₅ heteroalkylene. In some embodiments, L¹ is substituted or unsubstituted C₅-C₁₂ heteroalkylene.

In some embodiments, each of L² is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR'S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L² is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L² is —O—, —NH—, —S(═O)—, —S(═O)₂—, or —C(═O)—. In some embodiments, L² is —C(═O)NH— or —NHC(═O)—. In some embodiments, L² is substituted or unsubstituted C₃-C₁₅ cycloalkyl, or substituted or unsubstituted C₁-C₁₂ heterocycloalkyl. In some embodiments, L² is substituted or unsubstituted aryl or substituted or unsubstituted heteroaryl. In some embodiments, L² is substituted or unsubstituted C₁-C₃₀ alkylene. In some embodiments, L² is substituted or unsubstituted C₂-C₃₀ alkenylene. In some embodiments, L² is substituted or unsubstituted C₁-C₃₀ heteroalkylene. In some embodiments, L² is substituted or unsubstituted C₅-C₂₅ heteroalkylene. In some embodiments, L² is substituted or unsubstituted C₅-C₁₂ heteroalkylene.

In some embodiments, each of L³ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L³ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L³ is —O—, —NH—, —S(═O)—, —S(═O)₂—, or —C(═O)—. In some embodiments, L³ is —C(═O)NH— or —NHC(═O)—. In some embodiments, L³ is substituted or unsubstituted C₃-C₁₅ cycloalkyl, or substituted or unsubstituted C₁-C₁₂ heterocycloalkyl. In some embodiments, L³ is substituted or unsubstituted aryl or substituted or unsubstituted heteroaryl. In some embodiments, L³ is substituted or unsubstituted C₁-C₃₀ alkylene. In some embodiments, L³ is substituted or unsubstituted C₂-C₃₀ alkenylene. In some embodiments, L³ is substituted or unsubstituted C₁-C₃₀ heteroalkylene. In some embodiments, L³ is substituted or unsubstituted C₅-C₂₅ heteroalkylene. In some embodiments, L³ is substituted or unsubstituted C₅-C₁₂ heteroalkylene. In some embodiments, L³ is absent.

In some embodiments, each of L⁴ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁴ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁴ is —O—, —NH—, —S(═O)—, —S(═O)₂—, or —C(═O)—. In some embodiments, L⁴ is —C(═O)NH— or —NHC(═O)—. In some embodiments, L⁴ is substituted or unsubstituted C₃-C₁₅ cycloalkyl, or substituted or unsubstituted C₁-C₁₂ heterocycloalkyl. In some embodiments, L⁴ is substituted or unsubstituted aryl or substituted or unsubstituted heteroaryl. In some embodiments, L⁴ is substituted or unsubstituted C₁-C₃₀ alkylene. In some embodiments, L⁴ is substituted or unsubstituted C₂-C₃₀ alkenylene. In some embodiments, L⁴ is substituted or unsubstituted C₁-C₃₀ heteroalkylene. In some embodiments, L⁴ is substituted or unsubstituted C₅-C₂₅ heteroalkylene. In some embodiments, L⁴ is substituted or unsubstituted C₅-C₁₂ heteroalkylene. In some embodiments, L⁴ is absent.

In some embodiments, each of L⁵ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S) NR^{L}—, —CR^{L}—N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁵ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁵ is —O—, —NH—, —S(═O)—, —S(═O)₂—, or —C(═O)—. In some embodiments, L⁵ is —C(═O)NH— or —NHC(═O)—. In some embodiments, L⁵ is substituted or unsubstituted C₃-C₁₅ cycloalkyl, or substituted or unsubstituted C₁-C₁₂ heterocycloalkyl. In some embodiments, L⁵ is substituted or unsubstituted aryl or substituted or unsubstituted heteroaryl. In some embodiments, L⁵ is substituted or unsubstituted C₁-C₃₀ alkylene. In some embodiments, L⁵ is substituted or unsubstituted C₂-C₃₀ alkenylene. In some embodiments, L⁵ is substituted or unsubstituted C₁-C₃₀ heteroalkylene. In some embodiments, L⁵ is substituted or unsubstituted C₅-C₂₅ heteroalkylene. In some embodiments, L⁵ is substituted or unsubstituted C₅-C₁₂ heteroalkylene. In some embodiments, L⁵ is absent. In some embodiments, the linker comprises one or more selected from AEEA, AEEP, AEEEP, and AEEEEP groups. In some embodiments, the linker comprises

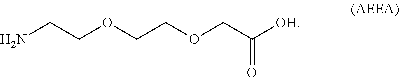 [SMILES: NCCOCCOCC(=O)O]

In some embodiments, the linker comprises

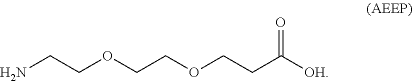 [SMILES: NCCOCCOCCC(=O)O]

In some embodiments, the linker comprises

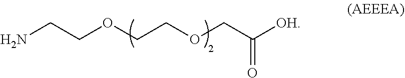

In some embodiments, the linker comprises

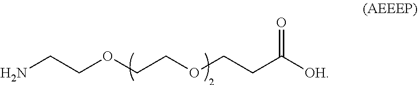

In some embodiments, the linker comprises

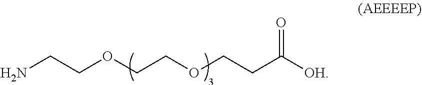

In some embodiments, a linker of the present disclosure is

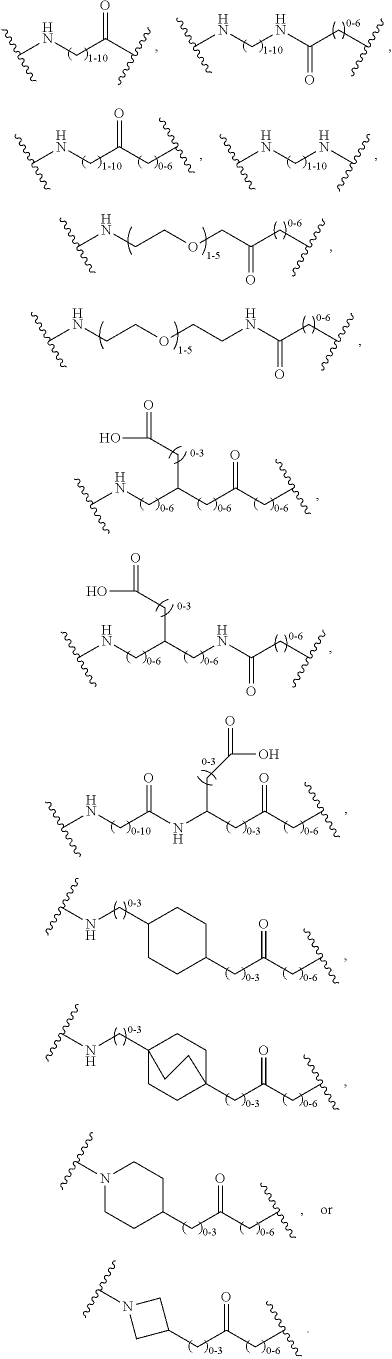

In some embodiments, a linker of the present disclosure is or comprises

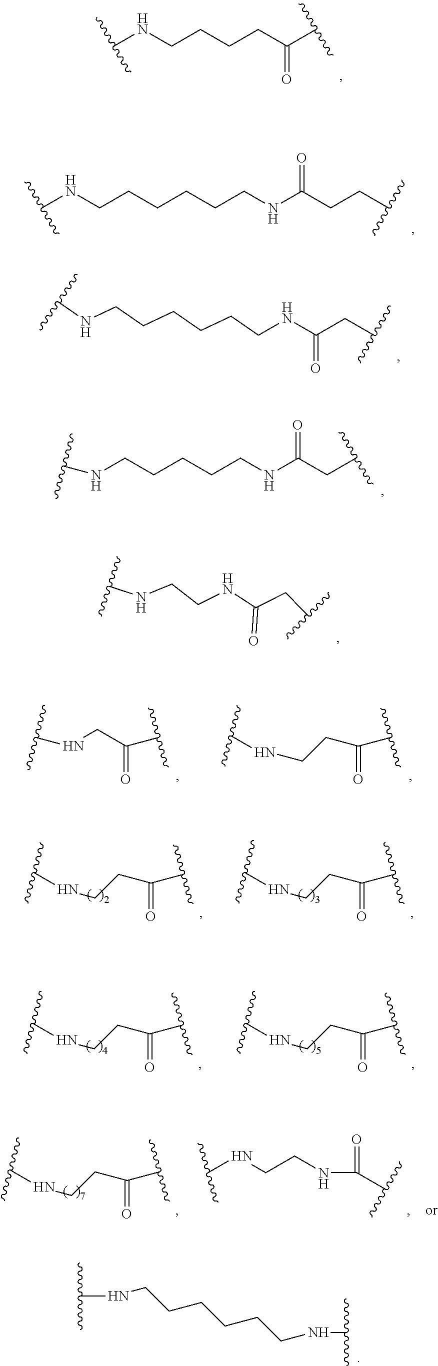

In some embodiments, a linker of the present disclosure is or comprises

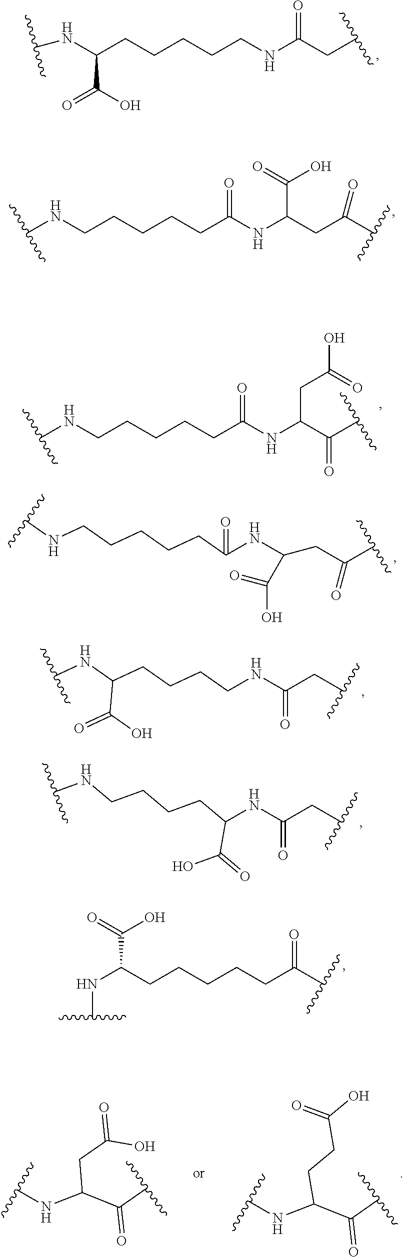

In some embodiments, a linker of the present disclosure is or comprises

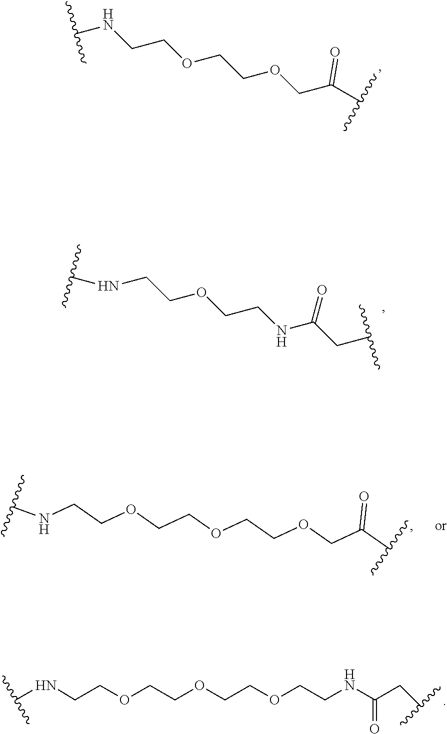 [SMILES: *NCCOCCOCC(*)=O ; *CC(=O)NCCOCCN* ; *NCCOCCOCCOCC(*)=O ; *CC(=O)NCCOCCOCCOCCN*]

In some embodiments, a linker of the present disclosure is or comprises

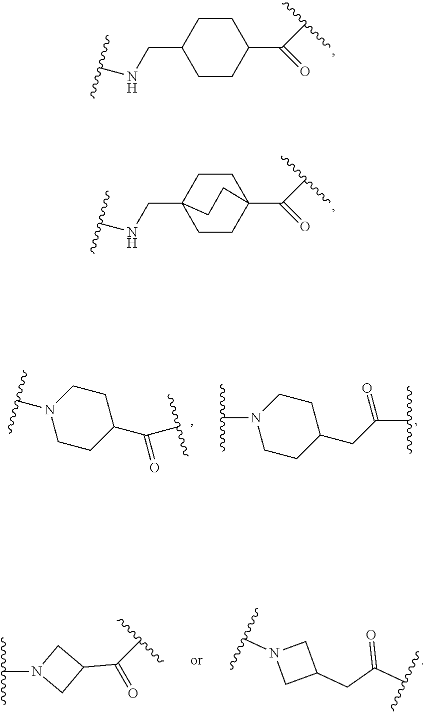

In some embodiments, the linker is a bond or

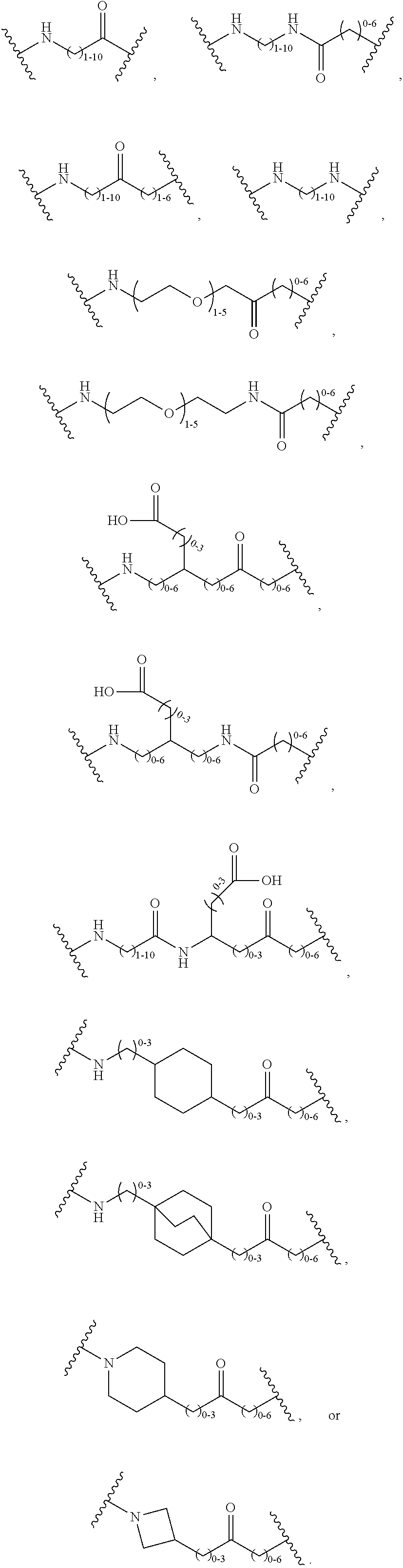

In some embodiments, the linker is:
(i) a bond;

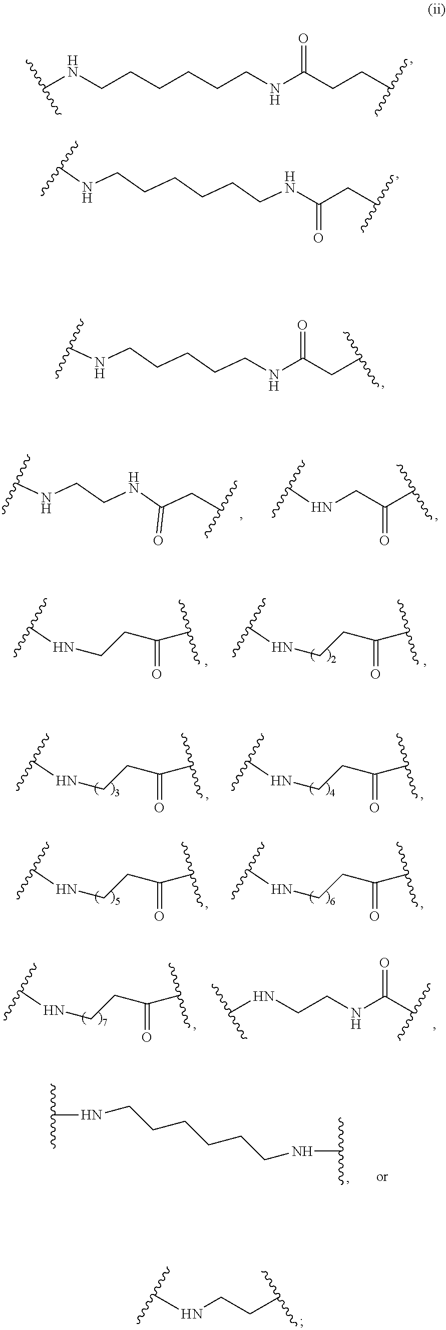

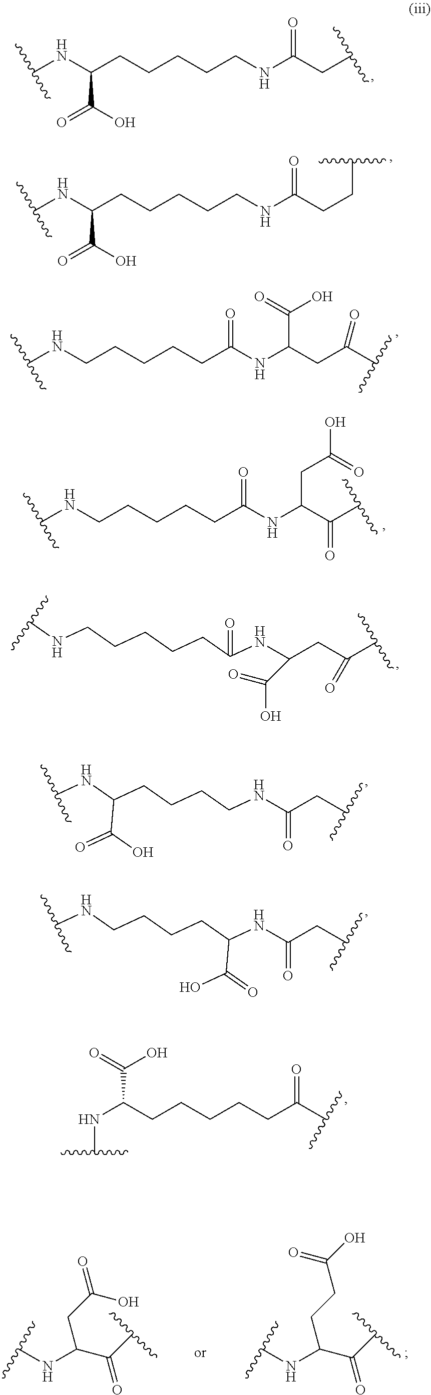

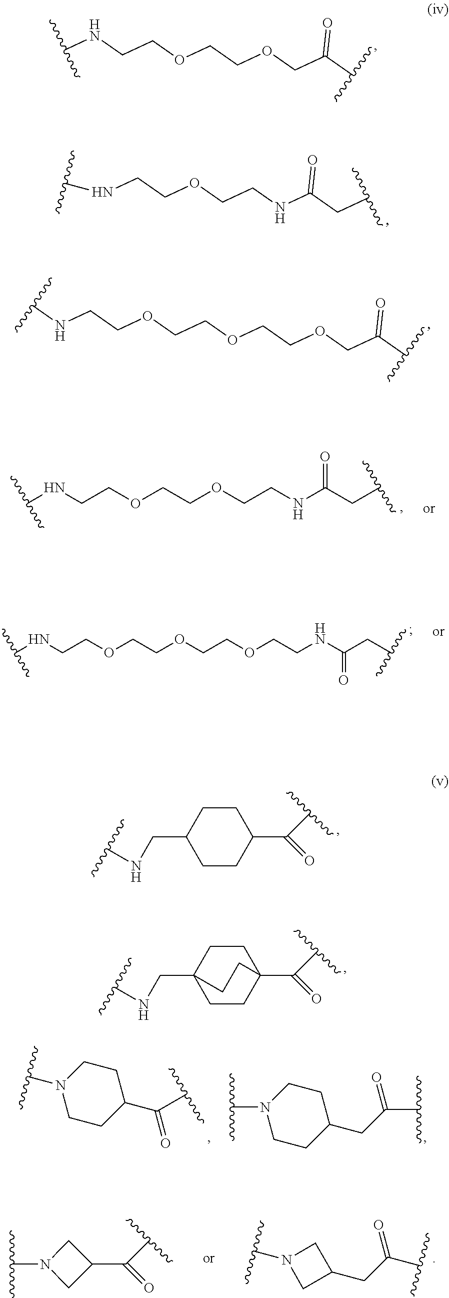

In some embodiments, a linker of the present disclosure comprises 1 to 20 groups independently selected from —CR^{b}R^{b}—, —C(═O)—, —S(═O)—, —S(═O)₂—, —NR^{a}—,

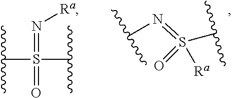

—CR^{b}═CR^{b}—, —C≡C—, —O—, —S—, —C(═O)O—, —OC(═O)—, —C(═O)NR^{a}—, —NR^{a}C(═O)—, —S(═O)₂NR^{a}—, —NR^{a}S(═O)₂—, —NR^{a}C(═O)NR^{a}—, —NR^{a}C(═O)O—, —OC(═O)NR^{a}—, arylene, heteroarylene, wherein each R^{a} is independently hydrogen, halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₆cycloalkyl, C₂-C₆heterocycloalkyl, aryl, or heteroaryl, and
wherein each R^{b} is independently hydrogen, halogen, —CN, —NO₂, —OR^{a}, —SR^{a}, C₁C₆alkyl, C₁C₆haloalkyl, C₁-C₆hydroxyalkyl, C₁C₆aminoalkyl, C₁C₆heteroalkyl, C₂-C₆alkenyl, C₂-C₆alkynyl, C₃-C₆cycloalkyl, C₂-C₆heterocycloalkyl, aryl, or heteroaryl. In some embodiments, a linker of the present disclosure comprises 1 to 5, 1 to 3, or 1 to 10 groups as described above.

In some embodiments, the linker is a bond.

### Metal Chelator

In one aspect, described herein are conjugates that comprise a metal chelator that is configured to bind with a radionuclide, for example, a conjugate of Formula (III), (IV1), (IV2), (IV3), (IV4), (IV5), and (IV6). The metal chelator can refer to a moiety of the conjugate that is configured to bind with a radionuclide. In some embodiments, a conjugate described herein comprises two or more independent metal chelators, e.g., 2, 3, 4, 5, or more metal chelators. In some embodiments, a conjugate described herein comprises two metal chelators, which can be the same or different. In some embodiments, a conjugate described herein comprises two or more metal chelators. In some embodiments, the conjugate comprises two radionuclides bound to the metal chelators. The metal chelator can be attached to the linker or the peptide through any suitable group/atom of the chelator.

In some embodiments, the metal chelator is capable of binding a radioactive atom. The binding can be direct, e.g., the metal chelator can make hydrogen bonds or electrostatic interactions with the radioactive atom. The binding can also be indirect, e.g., the metal chelator binds to a molecule that comprises a radioactive atom. In some embodiments, the metal chelator comprises, or is, a macrocycle. In some embodiments, the metal chelator comprises, or is, 2,2′,2″,2′″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more N, 4 or more carboxylic acid groups, or a combination thereof. In some embodiments, the metal chelator does not comprise S. In some embodiments, the metal chelator comprises a ring. In some embodiments, the ring comprises an O and/or an N. In some embodiments, the metal chelator is a ring that includes 3 or more N, 3 or more carboxylic acid groups, or a combination thereof. In some embodiments, the metal chelator is polydentate.

In some embodiments, a metal chelator described herein is selected from: DOTA, DOTA-GA, pBn-DOTA, pBn-SCN-DOTA, NH₂-DOTA, NH₂-DOTA-GA, p-NCS-Bn-DOTA-GA, p-NH₂-Bn-oxo-DO3A, p-SCN-Bn-oxo-DO3A, NOTA, NODA-GA, NH₂-NODA-GA, p-NCS-Bn-NODA-GA, p-NH₂-Bn-NOTA, p-SCN-Bn-NOTA, NCS-MP-NODA, NH₂-MPAA-NODA, PCTA, p-NH₂-Bn-PCTA, p-SCN-Bn-PCTA, p-SCN-Bn-HEHA, H2-MACROPA-NCS, H1-MACROPA, H2-MACROPA-NH₂, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA. In some embodiments, a metal chelator described herein is selected from: DOTA, DOTA-GA, pBn-DOTA, pBn-SCN-DOTA, NH₂-DOTA, NH₂-DOTA-GA, p-NCS-Bn-DOTA-GA, p-NH₂-Bn-oxo-DO3A, p-SCN-Bn-oxo-DO3A, NOTA, NODA-GA, NH₂-NODA-GA, p-NCS-Bn-NODA-GA, p-NH₂-Bn-NOTA, p-SCN-Bn-NOTA, NCS-MP-NODA, NH₂-MPAA-NODA, PCTA, p-NH₂-Bn-PCTA, p-SCN-Bn-PCTA, p-SCN-Bn-HEHA, H2-MACROPA-NCS, H1-MACROPA, H2-MACROPA-NH₂, H4-OCTAPA, tetra-(S, S, S, S)-Me-DOTA, tetra-(S, S, S, S)-Et-DOTA, tetra-(S, S, S, S)-iBu-DOTA, PYTA, or maleimide-nBu-DOTA.

In some embodiments, a metal chelator described herein has a structure of

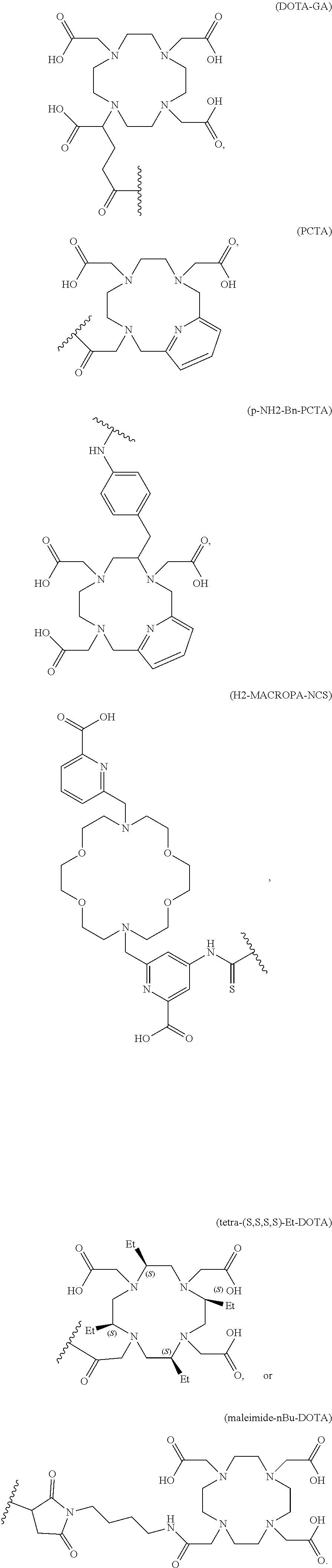

In some embodiments, a metal chelator described herein has a structure of

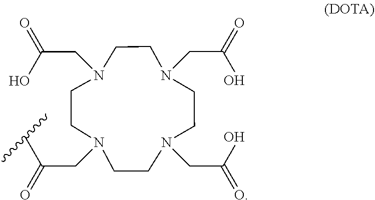 [SMILES: *C(=O)CN1CCN(CC(=O)O)CCN(CC(=O)O)CCN(CC(=O)O)CC1]

In some embodiments, a metal chelator described herein has a structure of

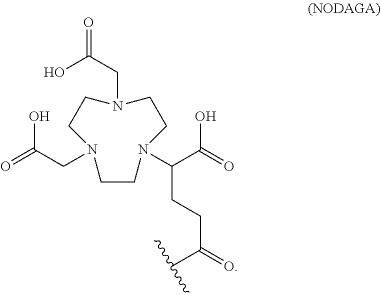 [SMILES: *C(=O)CCC(C(=O)O)N1CCN(CC(=O)O)CCN(CC(=O)O)CC1]

In some embodiments, a metal chelator described herein has a structure of

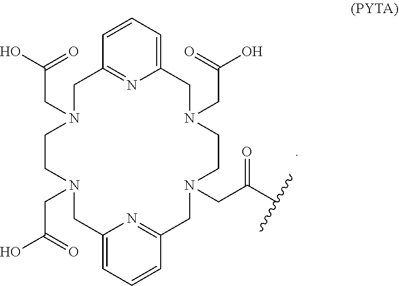 [SMILES: *C(=O)CN1CCN(CC(=O)O)Cc2cccc(n2)CN(CC(=O)O)CCN(CC(=O)O)Cc2cccc(n2)C1]

In some embodiments, a metal chelator described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH₂-Bn-Oxo-DO3A, DOTA, DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP, DOTAGA, DOTAGA-anhydride, DOTMA, DOTASA, DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-Bn1B-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H₃L¹, H₃L⁴, H₂azapa, H₅decapa, bispa², H₄pypa, H₄octapa, H₄CHXoctapa, p-SCN-Bn-H₄octapa, p-SCN-Bn-H₄octapa, TTHA, p-NO₂-Bn-neunpa, H₄octox, H₄macropa, H₂bispa², H₄phospa, H₆phospa, p-SCN-Bn-H₆phospa, TETA, p-NO₂-Bn-TETA, TRAP, TPA, HBED, SHBED, HBED-CC, (HBED-CC) TFP, DMSA, DMPS, DHLA, lipoic acid, TGA, BAL, Bis-thioseminarabazones, p-SCN-NOTA, nNOTA, NODAGA, CB-TE1A1P, 3P-C-NETA-NCS, 3p-C-DEPA, 3P-C-DEPA-NCS, TCMC, PCTA, NODIA-Me, TACN, pycup1A1B, pycup2A, THP, DEDPA, H2DEDPA, p-SCN-Bn-H2DEDPA, p-SCN-Bn-TCMC, motexafin, NTA, NOC, 3p-C-NETA, p-NH₂-Bn-TE3A, SarAr, DiAmSar, SarAr-NCS, AmBaSar, BaBaSar, TACN-TM, CP256, C-NE3TA, C-NE3TA-NCS, NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACHPYR, NEC-SP, L^{py}, L1, L2, L3, and EuK-106. In some embodiments, the metal chelator is DOTA, TRITA, TETA, DOTA-MA, DO3A-HP, DOTMA, DOTA-pNB, DOTP, DOTMP, DOTEP, DOTMPE, F-DOTPME, DOTPP, DOTBzP, DOTA-monoamide, p-NCS-DOTA, p-NCS-PADOTA, BAT, DO3TMP-Monoamide, p-NCS-TRITA, NOTA, or CHX-A″-DTPA. In some embodiments, a metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, a metal chelator described herein comprises DOTA, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP. In some embodiments, a metal chelator described herein comprises H₄pypa, H₄octox, H₄octapa, p-NO₂-Bn-neunpa, p-SCN-Bn-H₄eunpa, TTHA, ^{t}Bupypa-C7-NHS, H₄neunpa, H₂macropa, HP-DO3A, BT-DO3A, DO3A-Nprop, DO3AP, DO2A2P, DOA3P, DOTP, DOTPMB, DOTAMAE, DOTAMAP, DO3AMBu, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, or HBED.

In some embodiments, the metal chelator is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4]arene-tetracarboxylic acid. In some embodiments, the metal chelator is macropa. In some embodiments, the metal chelator is macropa-NCS. In some embodiments, the metal chelator is H₄pypa. In some embodiments, the metal chelator is H₄octapa. In some embodiments, the metal chelator is H₄CHXoctapa. In some embodiments, the metal chelator is DOTP. In some embodiments, the metal chelator is crown. In some embodiments, the metal chelator is NOTA. In some embodiments, the metal chelator is NODAGA.

In some embodiments, the metal chelator is DOTA. In some embodiments, the metal chelator is a chiral derivative of DOTA. Exemplary chiral DOTA chelators are described in Dai et al., Nature Communications (2018) 9:857. In some embodiments, the metal chelator is 2,2′,2″,2′-((2S,5S,8S,11S)-2,5,8,11-tetramethyl-1,4,7,10-tetraazacyclododecane-1,4,7,10-tetrayl)tetraacetic acid. In some embodiments, the metal chelator has a structure of

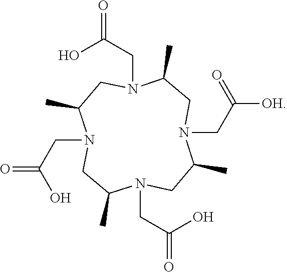 [SMILES: C[C@H]1CN(CC(=O)O)[C@@H](C)CN(CC(=O)O)[C@@H](C)CN(CC(=O)O)[C@@H](C)CN1CC(=O)O]

In some embodiments, the metal chelator is 2,2′,2″,2′-((2S,5S,8S,11S)-2,5,8,11-tetraethyl-1,4,7,10-tetraazacyclododecane-1,4,7,10-tetrayl)tetraacetic acid. In some embodiments, the metal chelator has a structure of

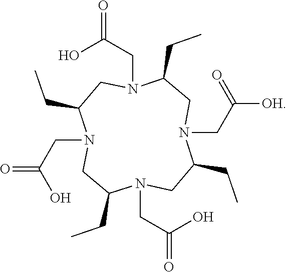 [SMILES: CC[C@H]1CN(CC(=O)O)[C@@H](CC)CN(CC(=O)O)[C@@H](CC)CN(CC(=O)O)[C@@H](CC)CN1CC(=O)O]

In some embodiments, the metal chelator has a structure of

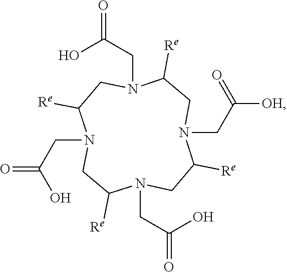

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments, the metal chelator has a structure of

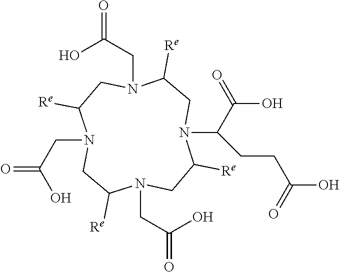

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain.

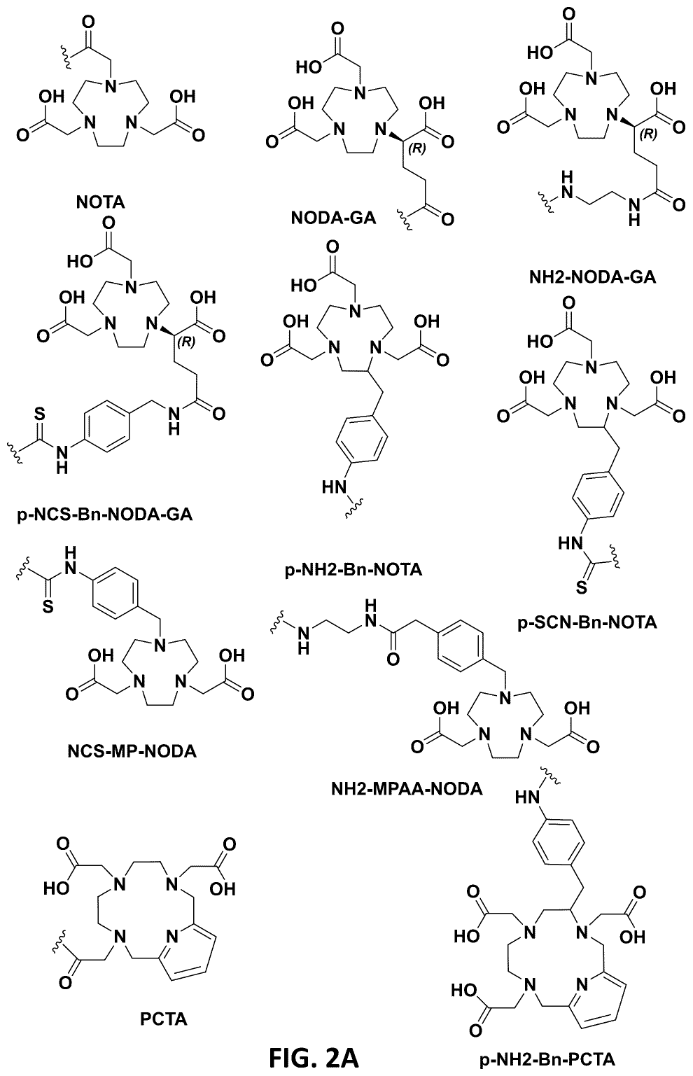

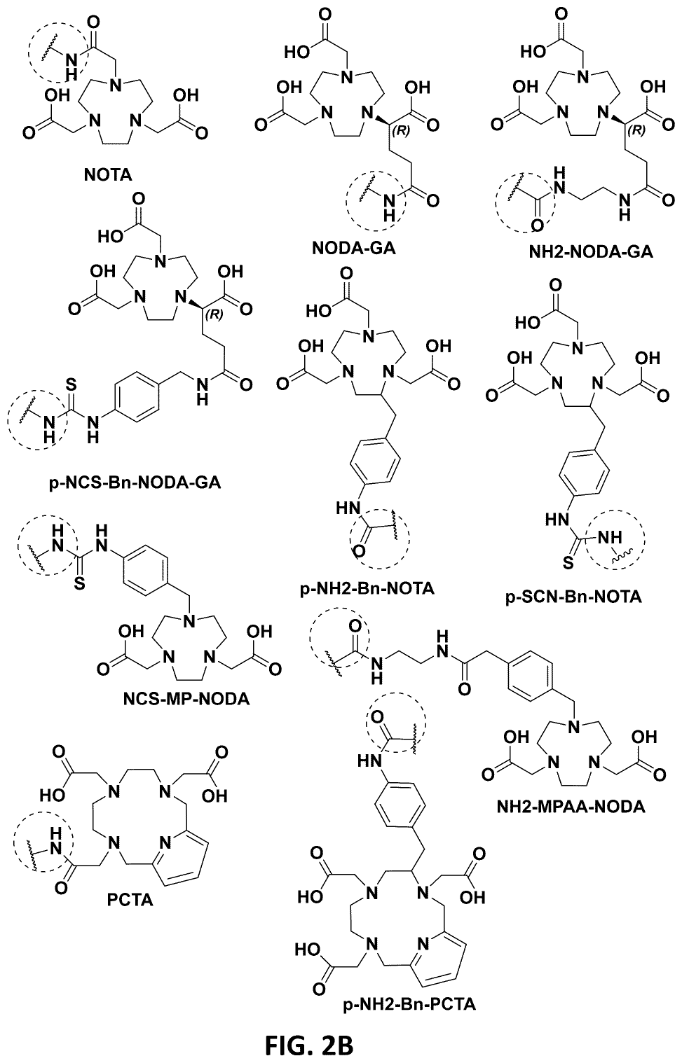

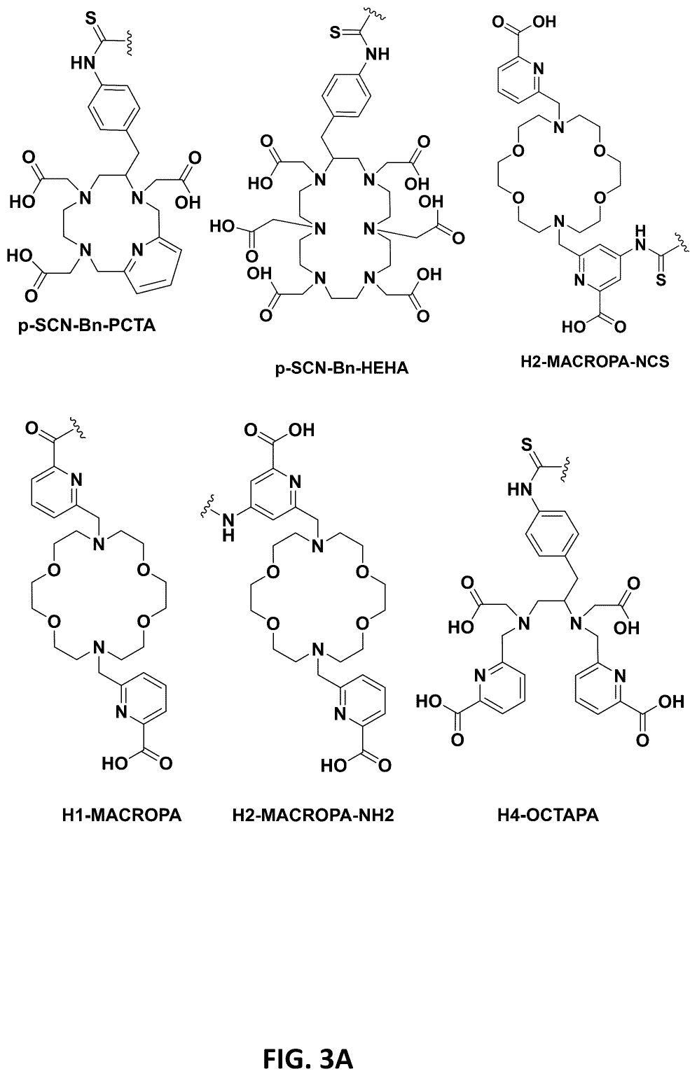

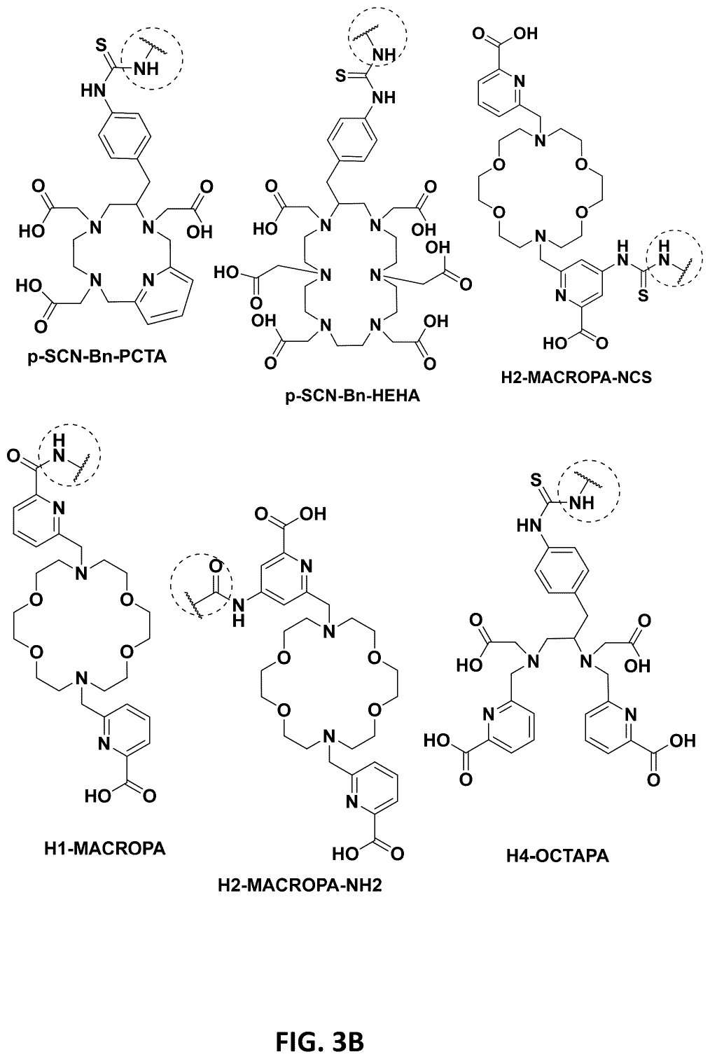

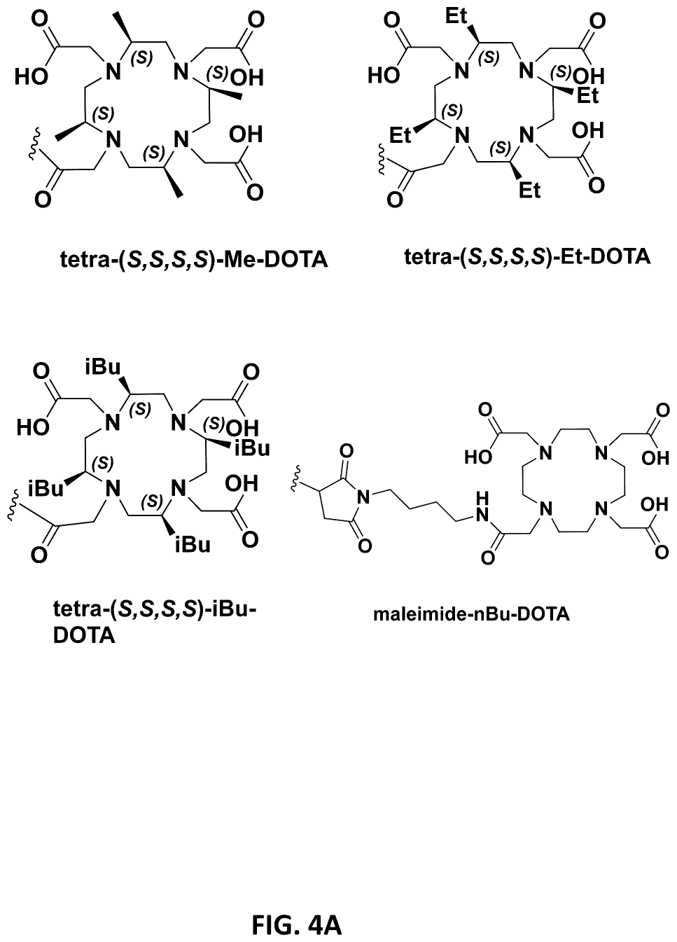

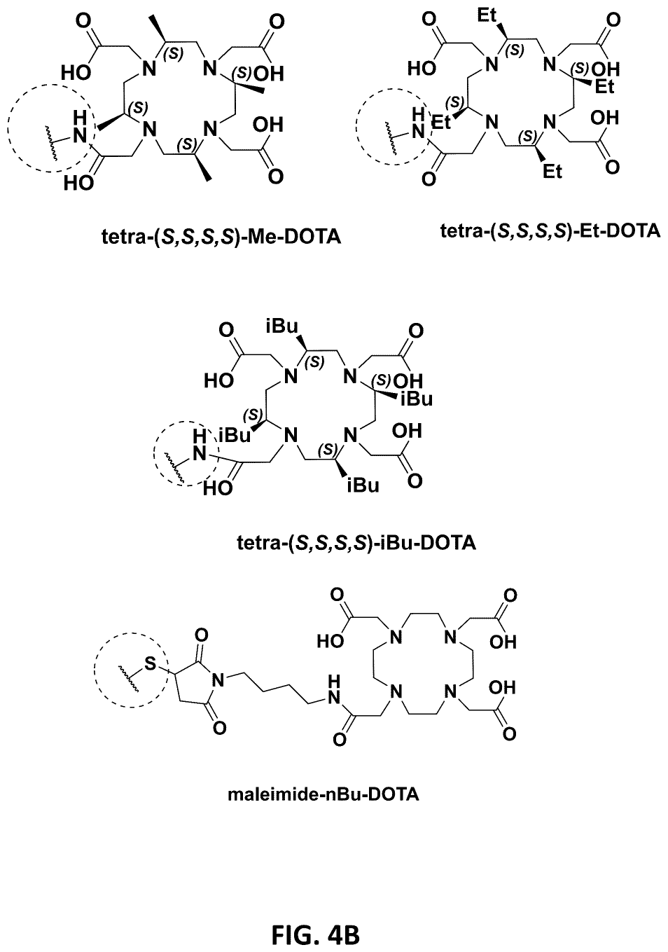

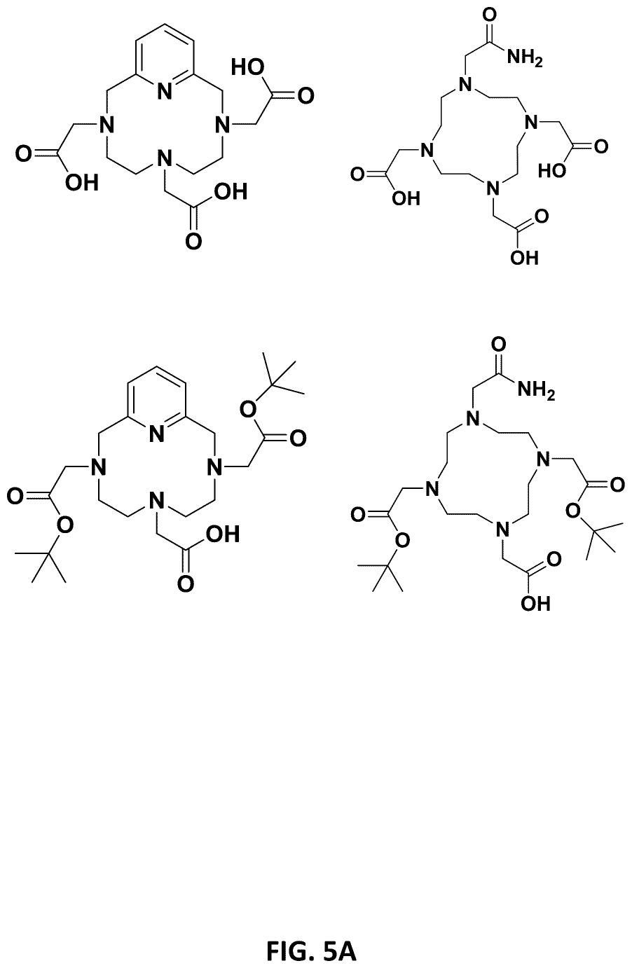

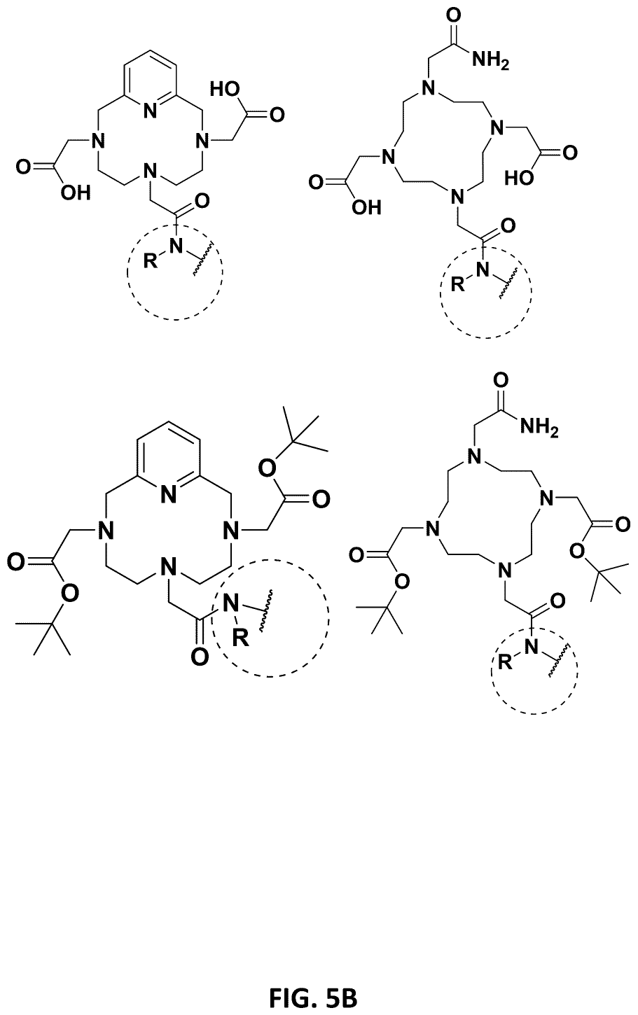

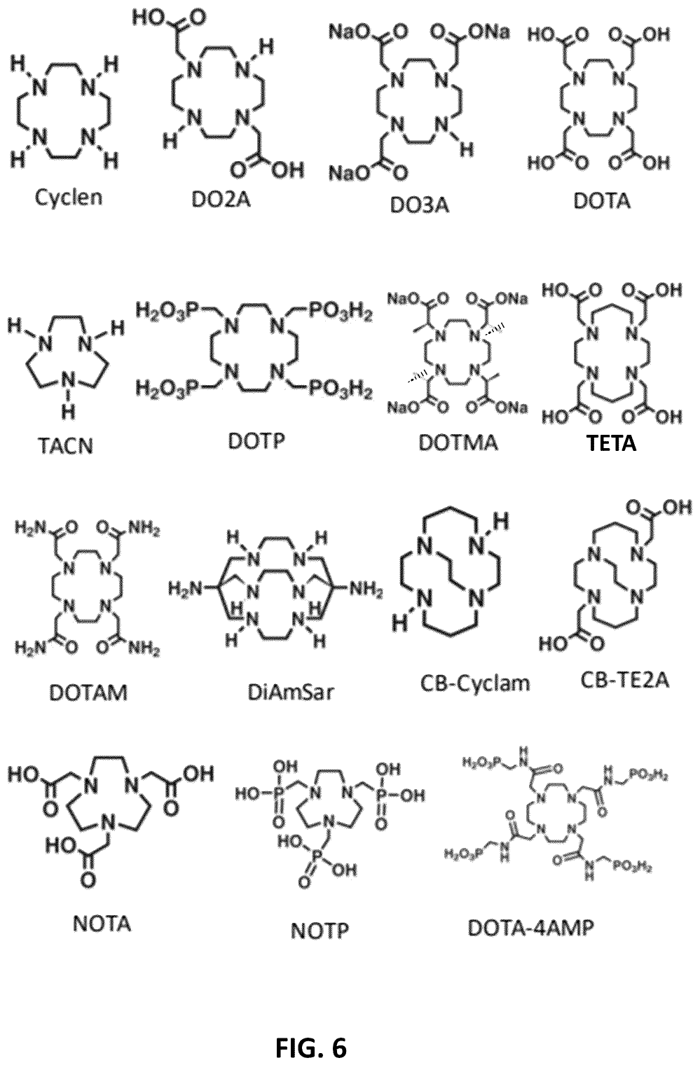

In some embodiments, the conjugate comprises DOTA. In some embodiments, the conjugate comprises a DOTA derivative such as p-SCN-Bn-DOTA and MeO-DOTA-NCS. In some embodiments, the conjugate comprises two independent metal chelators, and at least one or both are DOTA. The structures of some exemplary metal chelators are illustrated in FIGS. 6-25 (without showing the attachment points). Exemplary metal chelators are also illustrated in FIGS. 1A, 2A, 3A, 4A, and 5A (attachment point shown as a squiggly line) and FIGS. 1B, 2B, 3B, 4B and 5B (except that a part of the linker or the peptide covalently connected to the metal chelator is shown in the dashed circle). In some embodiments, a conjugate comprises a metal chelator of FIG. 1A. In some embodiments, a conjugate comprises a metal chelator of FIG. 2A. In some embodiments, a conjugate comprises a metal chelator of FIG. 3A. In some embodiments, a conjugate comprises a metal chelator of FIG. 4A. In some embodiments, a conjugate comprises a metal chelator of FIG. 5A. Exemplary metal chelators are further described in WO2012/174136; US20130183235A1; US20120219495A1; U.S. Pat. No. 5,334,371, EP292689 A2, WO2023202655 A1, Ramogidaand et al., EJNMMI radiopharm. chem. 4, 21 (2019); Thiele et al., Cancer Biotherapy and Radiopharmaceuticals 2018; Li et al., Bioconjugate Chem. 2019, 30, 5, 1539-1553; and Baranyai et al., Eur. J. Inorg. Chem. 36-56 (2020), each of which is incorporated by reference in its entirety.

A metal chelator such as DOTA can interact with a radionuclide (e.g., 177Lu or 225 Ac) via one or more functional groups and/or atoms. For example, a metal chelator can interact with a radionuclide via nitrogen and/or oxygen atoms. As another example, a metal chelator can interact with a radionuclide via carbonyl, carboxylic acid, amino, and/or amide groups of the metal chelator. In some embodiments, the interaction of a metal chelator and a radionuclide of the conjugates disclosed herein can be illustrated as

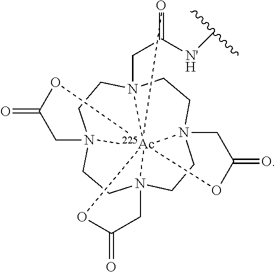

In some embodiments, the interaction of a metal chelator and a radionuclide of the conjugates disclosed herein can be illustrated as

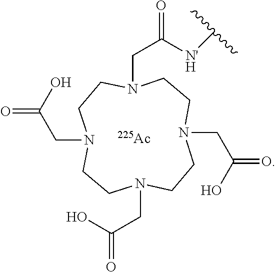

In some embodiments, the interaction of a metal chelator and a radionuclide of the conjugates disclosed herein can be illustrated as

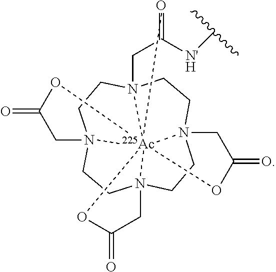

In some embodiments, the interaction of a metal chelator and a radionuclide of the conjugates disclosed herein can be illustrated as

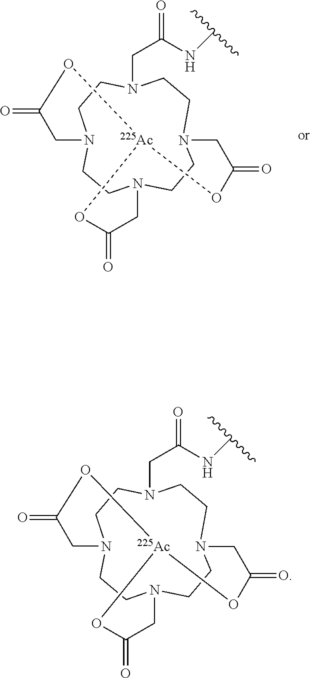

In some embodiments, the interaction of a metal chelator and a radionuclide of the conjugates disclosed herein can be illustrated as

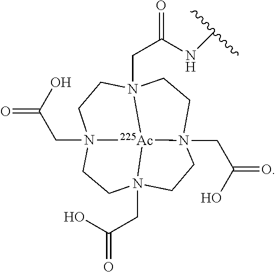

In some embodiments, the interaction of a metal chelator and a radionuclide of the conjugates disclosed herein can be illustrated as

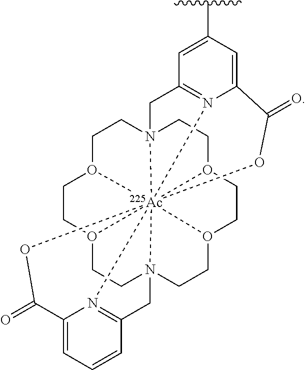

In some embodiments, the radionuclide exists in a positive oxidation state e.g., ²²⁵Ac³⁺, ¹⁷⁷Lu³⁺. In some embodiments, for example in certain aqueous conditions, the radionuclide exists in a salt form, e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the conjugate is in a salt form. In some embodiments, one or more of the carboxylic acid groups of the conjugate may exist as carboxylate anions. In some embodiments, one or more of the carboxylate anions of the conjugate may coordinate to the radionuclide. A person of ordinary skill would appreciate that the dissociation of an acid can depend on the pH value of the environment and its pK value. Accordingly, in some embodiments, a conjugate described herein can exist in a completely ionized, partially ionized or non-ionized form.

### Radionuclide

In one aspect, disclosed herein are radiopharmaceutical conjugates comprising a radionuclide. In some embodiments, the radionuclide is chelated or bound to a metal chelator. In some embodiments, the radionuclide is covalently bound to the conjugate. Generally, the type of radionuclide used in a therapeutic radiopharmaceutical can be tailored to the specific type of cancer, the type of targeting moiety (e.g., binding peptide), etc. Radionuclides that undergo α-decay produce particles composed of two neutrons and two protons, and radionuclides that undergo β-decay emit energetic electrons from their nuclei. Some radionuclides can also undergo electron capture and emit auger electrons. In some embodiments, the conjugate comprises an alpha particle-emitting radionuclide. Alpha radiation can cause direct, irreparable double-strand DNA breaks compared with gamma and beta radiation, which can cause single-stranded breaks via indirect DNA damage. The range of these particles in tissue and the half-life of the radionuclide can also be considered in designing the radiopharmaceutical conjugate. Table 4 below illustrates some properties of exemplary radionuclides.

TABLE 4
Exemplary radionuclides

| | | Exemplary application-- |
|---|---|---|
| | | Therapeutics (Tx) or |
| Radionuclide | Emission/Decay | Diagnostics (Dx) |
| Ac-225 | Alpha | Tx |
| Bi-209 | Alpha | Tx |
| Bi-212 | Alpha | Tx |
| Bi-213 | Alpha | Tx |
| Ce-134 | Electron Capture | Dx |
| Cu-61 | Positron | Dx |
| Cu-62 | Positron | Dx |
| Cu-64 | Positron | Dx |
| Cu-67 | Beta | Tx |
| Fr-223 | Alpha | Tx |
| Ga-68 | Positron | Dx |
| Gd-148 | Alpha | Tx |
| Ho-166 | Beta/gamma | Tx/Dx |
| In-111 | Beta/Electron Capture | Tx/Dx |
| Lu-177 | Beta | Tx |
| Pb-212 | Alpha | Tx |
| Pm-153 | Beta | Tx |
| Po-213 | Alpha | Tx |
| Ra-223 | Alpha | Tx |
| Ra-224 | Alpha | Tx |
| Rh-105 | Beta | Tx |
| Sm-153 | Beta | Tx |
| Tb-149 | Alpha | Tx |
| Tb-152 | Positron | Dx |
| Tb-161 | Beta | Tx/Dx |
| Tc-99m | Gamma/Isomeric Transition | Dx |
| Th-227 | Alpha | Tx |
| Th-229 | Alpha | Tx |
| Tm-167 | Beta/Electron Capture | Tx |
| Y-90 | Beta | Tx |
| Yb-175 | Beta | Tx |
| Zr-89 | Positron | Dx |
| At-211 | Alpha | Tx |

In some embodiments, the radiopharmaceutical conjugate described herein comprises a radionuclide selected from Table 4.

In some embodiments, the radiopharmaceutical conjugate described herein comprises one or more independent radionuclides. In some embodiments, the radiopharmaceutical conjugate comprises two radionuclides. In some embodiments, each of the one or more radionuclides is bound to the metal chelator of the radiopharmaceutical conjugate. In some embodiments, two radionuclides of the radiopharmaceutical conjugate are bound to the same metal chelator. In some embodiments, two radionuclides of the radiopharmaceutical conjugate are bound to two independent metal chelators. In some embodiments, each of the one or more radionuclides is an alpha particle-emitting radionuclide.

In some embodiments, the radiopharmaceutical conjugate described herein comprises an alpha particle-emitting radionuclide. In some embodiments, the radiopharmaceutical conjugate comprises an alpha-particle emitting radionuclide bound to the metal chelator. In some embodiments, the alpha particle-emitting radionuclide is actinium-225 (²²⁵Ac), radium-223 (²²³Ra), radium-224 (²²⁴Ra), bismuth-209 (²⁰⁹Bi), bismuth-213 (²¹³Bi), gadolinium-148 (¹⁴⁸Gd), terbium-149 (¹⁴⁹Tb), polonium-213 (²¹³Po), francium-223 (²²³Fr), thorium-227 (²²⁷Th), thorium-229 (²²⁹Th), or lead-212 (²¹²Bb). In some ¹⁴⁸Gd, embodiments, the alpha particle-emitting radionuclide is selected from ²²⁵Ac, ²²³Ra, ²⁰⁹Bi, ²¹³Bi, ¹⁴⁸Gd, ¹⁴⁹Th, ²¹³Po, ²²³ Fr, ²²⁷Th, ²²⁹Th, and ²¹²Pb. In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²⁷Th. In some embodiments, the alpha particle-emitting radionuclide is ¹⁴⁹Tb. In some embodiments, the conjugate comprises ²²⁵Ac. In some embodiments, the conjugate comprises two ²²⁵ Ac radionuclides. In some embodiments, the radionuclide is no-carrier added (i.e., non-carrier-added or n.c.a.) ¹⁷⁷Lu. In some embodiments, the radionuclide is no-carrier added (i.e., non-carrier-added or n.c.a.) ²²⁵Ac. In some embodiments, the radionuclide is ¹⁷⁷Lu free of long-lived radioactive contaminants and byproducts. In some embodiments, the conjugate comprises two ¹⁷⁷Lu radionuclides. In some embodiments, the radionuclide is a non-carrier-added radionuclide. In some embodiments, the radionuclide is a pseudo-radiometal. In some embodiments, the pseudo-radiometal is aluminum-[¹⁸F]fluoride ([¹⁸F]AlF) complex.

In some embodiments, the radiopharmaceutical conjugate described herein comprises a radionuclide selected from ⁶²Cu, ⁶⁴Cu, ⁶⁷Cu, ⁹⁰Y, ¹⁰⁹Pd, ¹¹¹Ag, ¹³⁴Ce, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹Au, ¹⁹⁹Au, ¹⁰⁵Rh, ¹¹¹Tb, ¹⁴⁹Pm, ¹⁵³Pm, ⁴⁴Sc, ⁴⁷Sc, ²¹³Po, ²¹²Pb, ²⁰⁹Bi, ²¹²Bi, ²¹³Bi, ²²⁵Ac, ^{117m}Sn, ⁶⁷Ga, ¹⁴⁹Tb, ¹⁵²Tb, ¹⁶⁷Tm, ¹⁷⁵Yb, ²²³Ra, ²²³Fr, ²²⁷Th, ²²⁹Th, ²⁰¹Tl, ¹⁴⁸Gd, ¹⁶⁰Gd, ¹⁴⁸Nd, ⁸⁹Sr, and ⁸⁹Zr. In some embodiments, the radionuclide is selected from ⁶²Cu, ⁶⁴Cu, ⁶⁷Cu, ⁶⁸Ga, ⁸⁹Zr, ⁹⁰Y, ^{99m}Tc, ¹⁰⁵Rh, ¹¹¹In, ¹³⁴Ce, ¹⁴⁸Gd, ¹⁴⁹Tb, ¹⁵²Tb, ¹⁵³Pm, ¹⁶⁷Tm, ¹⁷⁵Yb, ¹⁷⁷Lu, ²⁰⁹Bi, ²¹²Pb, ²¹³Po, ²¹³Bi, ²²³Ra, ²²³Fr, ²²⁷Th, ²²⁵Ac, and ²²⁹Th. In some embodiments, the radionuclide is 225 Ac. In some embodiments, the radionuclide is a decay daughter of ²²⁵Ac such as ²²¹Fr, ²¹⁷At, ²¹³Bi, ²¹³Po, ²⁰⁹Tl, ²⁰⁹Pb, or ²⁰⁹Bi. In some embodiments, the radiopharmaceutical conjugate comprises two ²²⁵Ac radionuclides. In some embodiments, the radionuclide is ¹⁷⁷Lu. In some embodiments, the radiopharmaceutical conjugate comprises two ¹⁷⁷Lu radionuclides. In some embodiments, the radionuclide is Ac-225 or Ga-68.

In some embodiments, the radiopharmaceutical conjugate described herein comprises a beta particle-emitting radionuclide. In some embodiments, the radiopharmaceutical conjugate comprises a beta particle-emitting radionuclide bound to the metal chelator. In some embodiments, the beta particle-emitting radionuclide is Cu-67, Lu-177, Y-90, Rh-105, Yb-175, Tm-167, Pm-153, Sm-153, Tb-161, or In-111. In some embodiments, the beta particle-emitting radionuclide is copper-67, rhodium-105, ytterbium-175, thulium-167, promethium-153, yttrium-90, samarium-153, or lutetium-177. In some embodiments, the beta particle emitting radionuclide is copper-67, yttrium-90, samarium-153, or lutetium-177. In some embodiments, the beta particle emitting radionuclide is lutetium-177.

In some embodiments, the radiopharmaceutical conjugate described herein comprises a gamma particle-emitting radionuclide. In some embodiments, the radiopharmaceutical conjugate comprises a gamma particle-emitting radionuclide bound to the metal chelator. In some embodiments, the gamma particle-emitting radionuclide is indium-111 or tin-117m.

In some embodiments, the radiopharmaceutical conjugate described herein comprises a positron particle-emitting radionuclide. In some embodiments, the radiopharmaceutical conjugate comprises a positron particle-emitting radionuclide bound to the metal chelator. In some embodiments, the positron-emitting radionuclide is gallium-68, copper-61, copper-62, copper-64, zirconium-89, or terbium-152. In some embodiments, the radionuclide is zirconium-89. In some embodiments, the radionuclide is gallium-68.

In some embodiments, a conjugate described herein comprises a radionuclide suitable for imaging or diagnostic purposes. In some embodiments, the radionuclide suitable for imaging is selected from ⁶²Cu, ⁶⁴Cu, ⁸⁹Zr, ¹³⁴Ce, ¹⁵²Tb, ⁶⁸Ga, ¹¹¹In, and ^{99m}Tc. In some embodiments, the radionuclide is suitable PET imaging. In some embodiments, the radionuclide suitable for PET imaging is selected from ⁶²Cu, ⁶⁴Cu, ⁸⁹Zr, ¹³⁴Ce, ¹⁵²Tb, and ⁶⁸Ga. In some embodiments, the radionuclide is suitable for SPECT imaging. In some embodiments, the radionuclide suitable for SPECT imaging is selected from ¹¹¹In and ^{99m}Tc.

In some embodiments, radiopharmaceutical conjugates described herein do not contain any hot radionuclide, i.e., a cold conjugate. For example, in some cases, a radionuclide can be replaced with a surrogate (e.g., ²²⁵Ac replaced with lanthanum) for testing and experimental purposes. In some embodiments, hot lutetium (Lu-177) is replaced with a cold lutetium (Lu-175).

In some embodiments, a radiopharmaceutical conjugate disclosed herein comprises a pseudo-radiometal, for example, an aluminum-18F complex. In some embodiments, the aluminum-18F complex is bound to a metal chelator.

### Conjugates Comprising Non-Radioactive Drugs

In one aspect, described herein is a conjugate comprising an SSTR binding peptide as described herein (e.g., a peptide of Formula (I), (I′), (I″), or Formula (II)), a non-radioactive drug, and optionally a linker. In some embodiments, disclosed herein is a conjugate of Formula (III), (IV1), (IV2), (IV3), (IV4), (IV5), or (IV6), except that the metal chelator is replaced with a non-radioactive drug. In some embodiments, the conjugate further comprises both a metal chelator and optionally a radionuclide bound to the metal chelator, and a non-radioactive drug. In some embodiments, the conjugate comprises an SSTR binding peptide herein (e.g., a peptide of Formula (I), (I′), (I″), or Formula (II)), a non-radioactive drug, and optionally a linker connecting the SSTR binding peptide to the non-radioactive drug. The non-radioactive drug can be a toxin. In some embodiments, the toxin is selected from pseudomonas exotoxin (PE), deBouganin, Bouganin, diphtheria toxin (DT) and ricin. In some embodiments, the non-radioactive drug can be a chemotherapy agent.

The non-radioactive drug can be a cytotoxic drug. Exemplary cytotoxic drugs include aplidin, azaribine, anastrozole, azacytidine, bleomycin, bortezomib, bryostatin-1, busulfan, calicheamycin, camptothecin, 10-hydroxycamptothecin, carmustine, celebrex, chlorambucil, cisplatin, irinotecan (CPT-11), SN-38, carboplatin, cladribine, cyclophosphamide, cytarabine, dacarbazine, docetaxel, dactinomycin, daunomycin glucuronide, daunorubicin, dexamethasone, diethylstilbestrol, doxorubicin, 2-pyrrolinodoxorubicin (2P-DOX), cyano-morpholino doxorubicin, doxorubicin glucuronide, epirubicin glucuronide, ethinyl estradiol, estramustine, etoposide, etoposide glucuronide, etoposide phosphate, floxuridine (FUdR), 3′,5′-O-dioleoyl-FudR (FUdR-dO), fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, 6-mercaptopurine, methotrexate, mitoxantrone, mithramycin, mitomycin, mitotane, phenyl butyrate, prednisone, procarbazine, paclitaxel, pentostatin, PSI-341, semustine streptozocin, tamoxifen, taxanes, taxol, testosterone propionate, thalidomide, thioguanine, thiotepa, teniposide, topotecan, uracil mustard, velcade, vinblastine, vinorelbine, vincristine, ricin, abrin, ribonuclease, onconase, rapLR1, DNase I, Staphylococcal enterotoxin-A, pokeweed antiviral protein, gelonin, diphtheria toxin, Pseudomonas exotoxin, Pseudomona endotoxin, or combinations of these.

In some embodiments, the non-radioactive drug is selected from duocarmycin and its analogues, dolastatins, combretastatin, calicheamicin, N-acetyl-□-calicheamycin (CMC), a calicheamycin derivative, maytansine and analogues thereof, DM-I, auristatin E, auristatin EB (AEB), auristatin EFP(AEFP), monomethyl auristatin E (MMAE), monomethyl auristatin F (MMAF), tubulysin, disorazole, the epothilones, Paclitaxel, docetaxel, Topotecan, echinomycin, estramustine, cemadotine, eleutherobin, methopterin, actinomycin, daunorubicin, the daunorubicin conjugates, mitomycin C, mitomycin A, vincristine, retinoic acid, camptothecin, a camptothecin derivative, SN38, maytansine, a derivative of the maytansinoid type, DM1, DM4, TK1, amanitin, a pyrrolobenzodiazepine, a pyrrolobenzodiazepine dimer, methotrexate, ilomedine, aspirin, an IMIDs, lenalidomide, pomalidomide.

In some embodiments, the non-radioactive drug is a polypeptide, a small molecule compound, a cell, a polynucleotide, a magnetic nanocluster, a nanoparticle, or a boron cluster.

In some embodiments, the non-radioactive drug is a polypeptide. In some embodiments, the non-radioactive drug is an antibody, a nanobody, or a functional fragment thereof. In some embodiments, the non-radioactive drug is a peptide ligand.

In some embodiments, the non-radioactive drug is a small molecule compound. In some embodiments, the small molecule therapeutic is a kinase inhibitor, an apoptosis inducer, a PROTAC, or a molecular glue. In some embodiments, the non-radioactive drug is an antineoplastic agent. In some embodiments, the antineoplastic agent is selected from an auristatin, a maytansinoid, a tubulysin, a cryptophycin, a hemisaterlin, a cemadotin, a rhizoxin, a discodermolide, a pyrrolobenzodiazepine, a duocarmycin, a calicheamicin, a camptothecin, an indolinobenzodiazepine, or an amatoxin. In some embodiments, the non-radioactive drug is a cell therapy such as an immune cell therapy or an engineered cell therapy.

In some embodiments, the non-radioactive drug is a polynucleotide such as a DNA or RNA oligonucleotide. In some embodiments, the non-radioactive drug is an aptamer.

In some embodiments, the non-radioactive drug is a ligand which binds to a second protein. In some embodiments, the second protein is CD137. In some embodiments, the ligand is a vitamin.

### Amino Acids

The structures of exemplary unnatural amino acids that are present in Table 1 and Table 2 can be found below. As described in Table 1, 2, or other tables, abbreviations have the following meanings: Upper case and lower case “D” means D-amino acids, e.g., D-Trp or dTrp refers to D-tryptophan; Me refers to a methyl group, e.g., NMe-Hcy represents N-Methyl-homocysteine (i.e., methyl-homocysteine); Ala or A refer to alanine; Asn or N refer to asparagine; Glu or E refer to glutamic acid; Asp or D refer to aspartic acid; Cys or C refer to cysteine; Gln or Q refer to glutamine; Gly or G refer to glycine; His or H refer to histidine; Ile or I refer to isoleucine; Leu or L refer to leucine; Lys or K refer to lysine; Phe or F refer to phenylalanine; Pro or P refer to proline; Ser or S refer to serine; Thr or T refer to threonine; Trp or W refer to tryptophan; Tyr or Y refer to tyrosine; and Val or V refer to valine.

Unless otherwise stated in the present specification, the following abbreviations for non-natural amino acids are used according to the following meanings:
(3-Azetidine)-hAla 2-amino-4-(azetidin-3-yl) butanoic acid, such as (S)-2-amino-4-(azetidin-3-yl) butanoic acid;
(5-Cl) D-Trp (R)-2-amino-3-(5-chloro-1H-indol-3-yl)propanoic acid;
(5-Me) D-Trp (R)-2-amino-3-(5-methyl-1H-indol-3-yl)propanoic acid;
(5-MeO) D-Trp (R)-2-amino-3-(5-methoxy-1H-indol-3-yl)propanoic acid;
(6-Me) D-Trp (R)-2-amino-3-(6-methyl-1H-indol-3-yl)propanoic acid;
(7-Me) D-Trp (R)-2-amino-3-(7-methyl-1H-indol-3-yl)propanoic acid;
(D/L) Hly 2,6-diamino-6-hydroxyhexanoic acid;
(R-βBenzyl) D-Trp (2R,3R)-2-amino-3-(1H-indol-3-yl)-4-phenylbutanoic acid;
(R-βBenzyl) Trp (2S,3R)-2-amino-3-(1H-indol-3-yl)-4-phenylbutanoic acid;
(R-βCyclopropyl) D-Trp (2R,3R)-2-amino-3-cyclopropyl-3-(1H-indol-3-yl)propanoic acid;
(R-βCyclopropyl) Trp (2S,3R)-2-amino-3-cyclopropyl-3-(1H-indol-3-yl)propanoic acid;
(R-βIsobutyl) D-Trp (2R,3R)-2-amino-3-(1H-indol-3-yl)-5-methylhexanoic acid;
(R-βIsobutyl) Trp (2S,3R)-2-amino-3-(1H-indol-3-yl)-5-methylhexanoic acid;
(R-βIsopropyl) D-Trp (2R,3R)-2-amino-3-(1H-indol-3-yl)-4-methylpentanoic acid;
(R-Isopropyl) Trp (2S,3R)-2-amino-3-(1H-indol-3-yl)-4-methylpentanoic acid;
(R-βMe) D-Trp (2S,3R)-2-amino-3-(1H-indol-3-yl)-4-methylpentanoic acid;
(R-βMe) Phe (2S,3R)-2-amino-3-phenylbutanoic acid;
(R-βMe) Trp (2S,3R)-2-amino-3-(1H-indol-3-yl) butanoic acid;
(R-βNeopentyl) D-Trp (2R,3R)-2-amino-3-(1H-indol-3-yl)-5,5-dimethylhexanoic acid;
(R-βNeopentyl) Trp (2S,3R)-2-amino-3-(1H-indol-3-yl)-5,5-dimethylhexanoic acid;
(R-βPhenyl) D-Trp (2R,3R)-2-amino-3-(1H-indol-3-yl)-3-phenylpropanoic acid;
(R-βPhenyl) Trp (2S,3R)-2-amino-3-(1H-indol-3-yl)-3-phenylpropanoic acid;
(R-βPropyl) D-Trp (2R,3R)-2-amino-3-(1H-indol-3-yl) hexanoic acid;
(R-βPropyl) Trp (2S,3R)-2-amino-3-(1H-indol-3-yl) hexanoic acid;
(R-βSecbutyl) D-Trp (2R,3R)-2-amino-3-(1H-indol-3-yl)-4-methylhexanoic acid;
(R-βSecbutyl) Trp (2S,3R)-2-amino-3-(1H-indol-3-yl)-4-methylhexanoic acid;
(S-βBenzyl) D-Trp (2R,3S)-2-amino-3-(1H-indol-3-yl)-4-phenylbutanoic acid;
(S-βBenzyl) Trp (2S,3S)-2-amino-3-(1H-indol-3-yl)-4-phenylbutanoic acid;
(S-βCyclopropyl) D-Trp (2R,3S)-2-amino-3-cyclopropyl-3-(1H-indol-3-yl)propanoic acid;
(S-βCyclopropyl) Trp (2S,3S)-2-amino-3-cyclopropyl-3-(1H-indol-3-yl)propanoic acid;
(S-βIsobutyl) D-Trp (2R,3S)-2-amino-3-(1H-indol-3-yl)-4-methylpentanoic acid;
(S-βIsobutyl) Trp (2S,3S)-2-amino-3-(1H-indol-3-yl)-4-methylpentanoic acid;
(S-βIsopropyl) D-Trp (2R,3S)-2-amino-3-(1H-indol-3-yl)-4-methylpentanoic acid;
(S-βIsopropyl) Trp (2S,3S)-2-amino-3-(1H-indol-3-yl)-4-methylpentanoic acid;
(S-βMe) D-Trp (2R,3S)-2-amino-3-(1H-indol-3-yl) butanoic acid;
(S-βMe) Phe (2S,3S)-2-amino-3-phenylbutanoic acid;
(S-βMe) Trp (2S,3S)-2-amino-3-(1H-indol-3-yl) butanoic acid;
(S-βNeopentyl) D-Trp (2R,3S)-2-amino-3-(1H-indol-3-yl)-5,5-dimethylhexanoic acid;
(S-βNeopentyl) Trp (2S,3S)-2-amino-3-(1H-indol-3-yl)-5,5-dimethylhexanoic acid;
(S-βPhenyl) D-Trp (2R,3S)-2-amino-3-(1H-indol-3-yl)-3-phenylpropanoic acid;
(S-βPhenyl) Trp (2S,3S)-2-amino-3-(1H-indol-3-yl)-3-phenylpropanoic acid;
(S-βPropyl) D-Trp (2R,3S)-2-amino-3-(1H-indol-3-yl) hexanoic acid;
(S-βPropyl) Trp (2S,3S)-2-amino-3-(1H-indol-3-yl) hexanoic acid;
(S-βSecbutyl) D-Trp (2R,3S)-2-amino-3-(1H-indol-3-yl)-4-methylhexanoic acid;
(S-βSecbutyl) Trp (2S,3S)-2-amino-3-(1H-indol-3-yl)-4-methylhexanoic acid;
(βGeminal methyl) Trp D-(R)-2-amino-3-(1H-indol-3-yl)-3-methylbutanoic acid;
(βGeminal methyl) Trp(S)-2-amino-3-(1H-indol-3-yl)-3-methylbutanoic acid;
2-aza-Trp 2-amino-3-(1H-indazol-3-yl)propanoic acid, such as (R)-2-amino-3-(1H-indazol-3-yl)propanoic acid;
3,3-diPhe 2-amino-3,3-diphenylpropanoic acid, such as (S)-2-amino-3,3-diphenylpropanoic acid;
3,5-diF Tyr 2-amino-3-(3,5-difluoro-4-hydroxyphenyl)propanoic acid, such as (S)-2-amino-3-(3,5-difluoro-4-hydroxyphenyl)propanoic acid;
3MeO-Phe 2-amino-3-(3-methoxyphenyl)propanoic acid, such as (S)-2-amino-3-(3-methoxyphenyl)propanoic acid;
3N-Tyr 2-amino-3-(6-hydroxypyridin-3-yl)propanoic acid, such as (S)-2-amino-3-(6-hydroxypyridin-3-yl)propanoic acid;
3Pal 2-amino-3-(pyridin-3-yl)propanoic acid, such as (S)-2-amino-3-(pyridin-3-yl)propanoic acid;
4-aminomethyl Phe 2-amino-3-(4-(aminomethyl)phenyl)propanoic acid, such as (S)-2-amino-3-(4-(aminomethyl)phenyl)propanoic acid;
4-aza-Trp 2-amino-3-(1H-pyrrolo[3,2-b]pyridin-3-yl)propanoic acid, such as (R)-2-amino-3-(1H-pyrrolo[3,2-b]pyridin-3-yl)propanoic acid;
4MeO-Phe 2-amino-3-(4-methoxyphenyl)propanoic acid, such as (S)-2-amino-3-(4-methoxyphenyl)propanoic acid;
4-oxa Lys O-(2-aminoethyl)-serine, such as O-(2-aminoethyl)-L-serine;
4-oxa NMe-Lys O-(2-aminoethyl)-N-methyl-serine, such as O-(2-aminoethyl)-N-methyl-L-serine;
4Pal 2-amino-3-(pyridin-4-yl)propanoic acid, such as (S)-2-amino-3-(pyridin-4-yl)propanoic acid;
5-aza-Trp 2-amino-3-(1H-pyrrolo[3,2-c]pyridin-3-yl)propanoic acid, such as (R)-2-amino-3-(1H-pyrrolo[3,2-c]pyridin-3-yl)propanoic acid;
5FY/5F-Tyr 2-amino-3-(3-fluoro-4-hydroxyphenyl)propanoic acid, such as (S)-2-amino-3-(3-fluoro-4-hydroxyphenyl)propanoic acid;
6-aza-Trp 2-amino-3-(1H-pyrrolo[2,3-c]pyridin-3-yl)propanoic acid, such as (R)-2-amino-3-(1H-pyrrolo[2,3-c]pyridin-3-yl)propanoic acid;
7-aza-Trp 2-amino-3-(1H-pyrrolo[2,3-b]pyridin-3-yl)propanoic acid, such as (R)-2-amino-3-(1H-pyrrolo[2,3-b]pyridin-3-yl)propanoic acid;
Alt allothreonine, such as L-allothreonine;
Aph(Hor) 2-amino-3-(4-(2,6-dioxohexahydropyrimidine-4-carboxamido)phenyl)propanoic acid, such as (2S)-2-amino-3-(4-(2,6-dioxohexahydropyrimidine-4-carboxamido)phenyl)propanoic acid;
azaLys amino (4-aminobutyl) carbamic acid;
Aza-Trp azatryptophan;
Bzt 2-amino-3-(benzo[b]thiophen-3-yl)propanoic acid, such as (S)-2-amino-3-(benzo[b]thiophen-3-yl)propanoic acid;
Cba 2-amino-3-cyclobutylpropanoic acid, such as (S)-2-amino-3-cyclobutylpropanoic acid;
Cba3N 2-amino-3-(azetidin-3-yl)propanoic acid, such as (S)-2-amino-3-(azetidin-3-yl)propanoic acid;
Cbg 2-amino-2-cyclobutylacetic acid, such as (S)-2-amino-2-cyclobutylacetic acid;
Cha 2-amino-3-cyclohexylpropanoic acid, such as (S)-2-amino-3-cyclohexylpropanoic acid;
Cha4N 2-amino-3-(piperidin-4-yl)propanoic acid, such as (S)-2-amino-3-(piperidin-4-yl)propanoic acid;
Cha4NH2 2-amino-3-(4-aminocyclohexyl)propanoic acid, such as (S)-2-amino-3-(4-aminocyclohexyl)propanoic acid;
Chg4N 2-amino-2-(piperidin-4-yl)acetic acid, such as (S)-2-amino-2-(piperidin-4-yl)acetic acid;
Cpg 2-amino-2-cyclopentylacetic acid, such as (S)-2-amino-2-cyclopentylacetic acid;
D-6F-Trp (R)-2-amino-3-(6-fluoro-1H-indol-3-yl)propanoic acid;
D-Aph(Cbm) (R)-2-amino-3-(4-ureidophenyl)propanoic acid;
D-Lys D-lysine;
D-Phg (R)-2-amino-2-phenylacetic acid;
D-Tpi (R)-2,3,4,9-tetrahydro-1H-pyrido[3,4-b]indole-3-carboxylic acid;
D-Trp D-tryptophan;
D-Tyr D-tyrosine;
F2CON 2-amino-3-(2-carbamoylphenyl)propanoic acid, such as (S)-2-amino-3-(2-carbamoylphenyl)propanoic acid;
F3CON 2-amino-3-(3-carbamoylphenyl)propanoic acid, such as (S)-2-amino-3-(3-carbamoylphenyl)propanoic acid;
F4COO 4-(2-amino-2-carboxyethyl)benzoic acid, such as (S)-4-(2-amino-2-carboxyethyl)benzoic acid;
G(cPr) 2-amino-2-cyclopropylacetic acid, such as (S)-2-amino-2-cyclopropylacetic acid;
L-DOPA 2-amino-3-(3,4-dihydroxyphenyl)propanoic acid, such as (S)-2-amino-3-(3,4-dihydroxyphenyl)propanoic acid;
Lys(diMe) N6,N6-dimethyl-lysine, such as N6,N6-dimethyl-L-lysine;
Lys(iPr) N6-isopropyl-lysine, such as N6-isopropyl-L-lysine;
Lys(Me) N6-methyl-lysine, such as N6-methyl-L-lysine;
Lys(triMe) N6,N6,N6-trimethyl-lysine, such as N6,N6,N6-trimethyl-L-lysine;
Mpd 2-amino-3-(methyl(phenyl)amino)propanoic acid, such as (S)-2-amino-3-(methyl(phenyl)amino)propanoic acid;
mTyr 2-amino-3-(3-hydroxyphenyl)propanoic acid, such as (S)-2-amino-3-(3-hydroxyphenyl)propanoic acid;
Nlys (4-aminobutyl)glycine;
NMe-Ala methyl-alanine, such as methyl-L-alanine;
NMe-Amp 2-(methylamino) heptanedioic acid, such as (S)-2-(methylamino) heptanedioic acid;
NMe-Asp methyl-L-aspartate, such as methyl-L-aspartate;
NMe-Azidolysine N6-diazo-N2-methyl-L-lysine, such as N6-diazo-N2-methyl-lysine;
NMe-Cha4N 2-amino-3-(piperidin-4-yl)propanoic acid, such as (S)-2-amino-3-(piperidin-4-yl)propanoic acid;
NMe-Chg4N 2-amino-2-(piperidin-4-yl)acetic acid, such as (S)-2-amino-2-(piperidin-4-yl)acetic acid;
NMe-Cys methyl-cysteine, such as methyl-L-cysteine;
NMe-Dab 4-amino-2-(methylamino) butanoic acid, such as (S)-4-amino-2-(methylamino) butanoic acid;
NMe-Dap 3-amino-2-(methylamino)propanoic acid, such as (S)-3-amino-2-(methylamino)propanoic acid;
NMe-D-Hcy methyl-D-homocysteine;
NMe-dLys methyl-D-lysine;
NMe-D-Trp methyl-D-tryptophan;
NMe-Glu methyl-glutamate, such as methyl-L-glutamate;
NMe-Hcy methyl-homocysteine, such as methyl-L-homocysteine;
NMe-hGlu 2-(methylamino) hexanedioic acid, such as (S)-2-(methylamino) hexanedioic acid;
NMe-hHcy 5-mercapto-2-(methylamino) pentanoic acid, such as (S)-5-mercapto-2-(methylamino) pentanoic acid;
NMe-hLys 7-amino-2-(methylamino) heptanoic acid, such as (S)-7-amino-2-(methylamino) heptanoic acid;
NMe-Hse methyl-homoserine, such as methyl-L-homoserine;
NMe-Hse (Se) methyl-homoselenocysteine, such as methyl-L-homoselenocysteine;
NMe-Lys methyl-lysine, such as methyl-L-lysine;
NMe-Nle methyl-norleucine, such as methyl-L-norleucine
NMe-Orn 5-amino-2-(methylamino) pentanoic acid, such as (S)-5-amino-2-(methylamino) pentanoic acid;
NMe-Phe methyl-phenylalanine, such as Methyl-L-phenylalanine;
NMe-propargyl alanine 2-methyl-2-(methylamino) pent-4-ynoic acid;
NMe-propargyl glycine 2-(methylamino) pent-4-ynoic acid;
Nva(NH—NH2) 2-amino-5-hydrazineylpentanoic acid, such as (S)-2-amino-5-hydrazineylpentanoic acid;
Phg 2-amino-2-phenylacetic acid, such as (S)-2-amino-2-phenylacetic acid;
Pic4 4-aminopiperidine-4-carboxylic acid;
PipzaA 2-amino-3-(piperazin-1-yl)propanoic acid, such as (S)-2-amino-3-(piperazin-1-yl)propanoic acid;
Ser(3-azetidine)O-(azetidin-3-yl)-serine, such as O-(azetidin-3-yl)-L-serine;
Ser(Ph) O-phenyl-serine, such as O-phenyl-L-serine;
Tme O-methyl-threonine, such as O-methyl-L-threonine;
trans-Hyp L-hydroxyproline;
Tyr(OBn) 2-amino-3-(4-(benzyloxy)phenyl)propanoic acid, such as (S)-2-amino-3-(4-(benzyloxy)phenyl)propanoic acid; and
Tyr(Phe) 2-amino-3-(4-phenoxyphenyl)propanoic acid, such as (S)-2-amino-3-(4-phenoxyphenyl)propanoic acid.

Amino acids used in the disclosed peptides can be substituted with similar amino acids. In some embodiments, an amino acid can be substituted with another amino acid with similar hydrophobicity. In some embodiments, an amino acid can be substituted with another amino acid with similar hydrophilicity. In some embodiments, an amino acid can be substituted with another amino acid with similar size. In some embodiments, an amino acid can be substituted with another amino acid with similar charge. In some embodiment, an amino acid can be substituted with another amino acid with a similar functional group. In some embodiments, an amino acid can be substituted with another amino acid with the same functional group.

In some embodiments, an amino acid described herein can be replaced with a derivative thereof. Examples of an amino acid substitution or derivative include derivatives having an amine, amide, ester, or carboxyl group as the C-terminus and/or N-terminus thereof. Additional examples of amino acid/peptide derivatives include those obtained by modification such as phosphorylation, alkylation (e.g., methylation), acetylation, adenylylation, ADP-ribosylation, or glycosylation and fused protein obtained by fusion with another peptide or protein. These derivatives can be prepared by those skilled in the art in a known manner or a method based thereon. An amino acid derivative further encompasses the amino acids that have the same functional groups but with different lengths of the side chain (e.g., LysAc vs. OrnAc and cysteine vs. homocysteine). An amino acid derivative further encompasses amino acids with a different aromatic moiety compared to the canonical amino acid (e.g., the indole in tryptophan vs the 7-azaindole in 7-AzaTrp; the phenyl in phenylalanine vs the pyridine in 4Py). An amino acid derivative further encompasses amino acids with optional substituents, i.e., optionally substituted amino acid.

In some embodiments, a derivative of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂, C₁-C₆ alkyl, C₁-C₆ heteroalkyl, C₁-C₆ alkoxy, C₀-C₁₀ aryl, C₃-C₆ cycloalkyl, 6- to 10-membered heterocycloalkyl, and 6- to 10-membered heteroaryl. In some embodiments, the derivative is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, and C₁-C₆ alkyl. In some embodiments, the derivative is selected from amino acids having one or two substituents based on the amino acid, and wherein the substituents are independently selected from halogen, —CN, —NH₂, —NH(C₁-C₃alkyl), —N(C₁-C₃alkyl)₂, and C₁-C₆ alkyl. In some embodiments, the derivative is selected from amino acids having one or two substituents based on the amino acid, and wherein the substituents are independently selected from C₁-C₆ alkyl.

In some embodiments, a derivative of an amino acid is selected from amino acids that have the similar hydrophilicity or hydrophobicity compared to the amino acid. Thus, in some embodiments, a positively charged amino acid can be a derivative of another positively charged amino acid. In some embodiments, a negatively charged amino acid can be a derivative of another negatively charged amino acid. In some embodiments, a zwitterionic amino acid can be a derivative of another zwitterionic amino acid.

In some embodiments, a hydrophilic amino acid has an electrically charged side chain. In some embodiments, a hydrophilic amino acid has a positive charge. In some embodiments, a hydrophilic amino acid has a negative charge. In some embodiments, a hydrophilic amino acid is zwitterionic (e.g., KCOpipzaa). In some embodiments, a hydrophilic amino acid comprises a —OH, COOH, —NH— or NH₂ moiety. In some embodiments, a hydrophilic amino acid comprises-OH, —C(O)OH, —NHC(═NH)NH₂, —NHC(O)NH₂, —C(O)NH₂, or —NHC(O)CH₃. In some embodiments, a hydrophilic amino acid comprises a side chain of C₁C₆hydroxyalkyl, C₁C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

In some embodiments, a hydrophobic amino acid is not charged. In some embodiments, a hydrophobic amino acid contains at least 2 contiguous carbon atoms. In some embodiments, a hydrophobic amino acid comprises at least 3 contiguous carbon atoms, either linear or branched. In some embodiments, a hydrophobic amino acid comprises at least 4 contiguous carbon atoms, either linear or branched. In some embodiments, a hydrophobic amino acid comprises at least 5 contiguous carbon atoms, either linear or branched. In some embodiments, a hydrophobic amino acid comprises an ethylene moiety in the side chain. In some embodiments, a hydrophobic amino acid comprises a propylene moiety in the side chain. In some embodiments, a hydrophobic amino acid comprises a butylene moiety in the side chain. In some embodiments, a hydrophobic amino acid comprises phenyl moiety. In some embodiments, a hydrophobic amino acid comprises a heteroaryl moiety. In some embodiments, a hydrophobic amino acid is Trp, Tyr, Phe, or derivatives thereof.

In some embodiments, a derivative of an amino acid is selected from amino acids that have the same functional group as the amino acid, and wherein the derivative has a different length of a side chain compared to the amino acid. In some embodiments, a derivative of an amino acid is selected from amino acids that have the same charge compared to the amino acid. In some embodiments, a derivative of an amino acid is selected from amino acids that have the same polarity compared to the amino acid. In some embodiments, an amino acid comprising an aromatic group can be a derivative of another amino acid having an aromatic group. In some embodiments, an amino acid comprising a phenyl can be a derivative of another amino acid having a phenyl. In some embodiments, an amino acid comprising a heteroaryl can be a derivative of another amino acid having a heteroaryl.

In some embodiments, an amino acid comprising a cycloalkyl group can be a derivative of another amino acid having a cycloalkyl group. In some embodiments, an amino acid comprising a heterocycloalkyl group can be a derivative of another amino acid having a heterocycloalkyl group.

In some embodiments, a derivative of an amino acid is selected from amino acids that have similar polarity and/or charge with the amino acid. For example, in some embodiments, a polar, uncharged amino acid can be a derivative of another polar, uncharged amino acid (e.g., Hgn, Q, S, T, Qglucamine),

In some embodiments, a derivative of an amino acid has the same number of hydrogen donor as the amino acid. In some embodiments, a derivative of an amino acid has the same number of hydrogen acceptor as the amino acid.

In some embodiments, the derivative has a molecular weight that does not vary for more than 14, 28, 30, 45 or 60 g/mol compared to the amino acid. In some embodiments, the derivative has a molecular weight that does not vary for more than 14 g/mol compared to the amino acid. In some embodiments, the derivative has a molecular weight that does not vary for more than 50 g/mol compared to the amino acid. In some embodiments, the derivative has a molecular weight that does not vary for more than 28 g/mol compared to the amino acid.

An amino acid derivative further encompasses amino acids wherein a functional group is substituted with another functional group having similar properties, e.g., a cysteine can be substituted with a homocysteine. In some embodiments, an aryl functional group can be substituted with an aryl or heteroaryl group. In some embodiments, a heteroaryl functional group can be substituted with an aryl or heteroaryl group. In some embodiments, an amino functional group can be substituted with an NH (alkyl) group.

As used herein, the expression “conservative amino acid substitution” refers to a substitution of functionally equivalent or similar amino acids. A conservative amino acid substitution in a peptide brings about a static change to the amino acid sequence of the peptide. For example, one or two or more amino acids having similar polarity act functionally equivalent to each other and bring about a static change in the amino acid sequence of the peptide. In general, a substitution within a certain group may be considered conservative regarding structure and function. However, as is clear to a person having ordinary skill in the art, the role played by a defined amino acid residue may be determined by its implication in the three-dimensional structure of the molecule containing the amino acid. For example, a cysteine residue in an oxidized-type (disulfide) form may have a lower polarity than that of a reduced-type (thiol) form. The long aliphatic part of the arginine side chain may constitute structurally and functionally important features. Furthermore, the side chain (tryptophan, tyrosine, phenylalanine) including an aromatic ring may contribute to ion-aromatic interaction or cation-pi interaction. In such a case, even if the amino acids having these side chains are substituted for amino acids belonging to the acidic or non-polar groups, they may be structurally and functionally conservative. There is a possibility that residues such as proline, glycine, cysteine (disulfide form) have a direct effect on the three-dimensional structure of the main chain and often may not be substituted without structural distortion.

Conservative amino acid substitution, as shown below, includes specific substitution based on the similarity of side chains (for example, substitutions are described in Lehninger, Biochemistry, Revised 2nd Edition, published in 1975, pp. 73 to 75: L. Lehninger, Biochemistry, 2nd edition, pp. 73 to 75, Worth Publisher, New York (1975)), incorporated herein by reference, and typical substitution.

Hydrophobic amino acids include amino acids that exhibit hydrophobicity, including alanine (also referred to as “Ala” or simply “A”), glycine (also referred to as “Gly” or simply “G”), valine (also referred to as “Val” or simply “V”), leucine (also referred to as “Leu” or simply “L”), isoleucine (also referred to as “Ile” or simply “I”), proline (also referred to as “Pro” or simply “P”), phenylalanine (also referred to as “Phe” or simply “F”), tryptophan (also referred to as Trp” or simply “W”), tyrosine (also referred to as “Tyr” or simply “Y”), and methionine (also referred to as “Met” or simply “M”).

Exemplary hydrophobic amino acids may be further divided into the following groups:
Aliphatic amino acids: Amino acids having a fatty acid or hydrogen in the side chain, including e.g., Ala, Gly, Val, Ile, and Leu.
Aliphatic/branched-chain amino acids: Amino acids having a branched fatty acid in the side chain, including e.g., Val, Ile, and Leu.
Aromatic amino acids: Amino acids having an aromatic ring in the side chain, including e.g., Trp, Tyr, and Phe.

In some embodiments, a hydrophobic amino acid has a C₁-C₈ alkyl, cycloalkyl, or heterocycloalkyl, wherein the alkyl, cycloalkyl, and heterocycloalkyl are each independently, optionally substituted. In some embodiments, a hydrophobic amino acid has a C₁-C₈ alkyl, cycloalkyl, or heterocycloalkyl, wherein the alkyl, cycloalkyl, and heterocycloalkyl are each unsubstituted. In some embodiments, a hydrophobic amino acid has a C₃-C₆ unsubstituted alkyl.

Hydrophilic amino acids include amino acids that exhibit hydrophilicity, including e.g., serine (also referred to as “Ser” or simply “S”), threonine (also referred to as “Thr” or simply “T”), cysteine (also referred to as “Cys” or simply “C”), asparagine (also referred to as “Asn” or simply “N”), glutamine (also referred to as “Gln” or simply “Q”), aspartic acid (also referred to as “Asp” or simply “D”), glutamic acid (also referred to as “Glu” or simply “E”), Elysine (also referred to as “Lys” or simply “K”), arginine (also referred to as “Arg” or simply “R”), and histidine (also referred to as “His” or “H”).

Exemplary hydrophilic amino acids may be further divided into the following groups:
Acidic amino acids: Amino acids whose side chains exhibit acidity, including Asp and Glu.
Basic amino acids: Amino acids whose side chains exhibit basicity, including Lys, Arg, and His.
Neutral amino acids: Amino acids whose side chains exhibit neutrality, including Ser, Thr, Asn, Gln, and Cys.

Exemplary hydrophilic amino acids include, for example, D, Q, E, S, N, T, C, H, or a derivative thereof.

Examples of the amino acids include natural protein L-amino acids, unnatural amino acids, and chemically synthesized compounds having properties known in the art as characteristics of an amino acid. Examples of the unnatural amino acids include, but not limited to, α,α-disubstituted amino acids (such as α-methylalanine), N-alkyl-α-amino acids, N-alkyl-β-amino acids, D-amino acids, β-amino acids, and α-hydroxy acids, each having a backbone structure different from that of natural amino acids; amino acids (such as norleucine and homohistidine) having a side-chain structure different from that of natural amino acids; amino acids (such as “homo” amino acids, homophenylalanine, and homohistidine) having extra methylene in the side chain thereof; and amino acids (such as cysteic acid) obtained by substituting a carboxylic acid functional amino group in the side chain thereof by a sulfonic acid group.

The peptides described herein can comprise one or more unnatural amino acids. Unnatural amino acids include, but are not limited to, (1) amino acids corresponding to an amino acid residue on a polypeptide subjected to modification after expression (ex. phosphorylated tyrosine, acetylated lysine, or farnesylated cysteine), (2) amino acids that cannot be used in expression on a ribosome but occur naturally, and (3) artificial amino acids that do not occur naturally (unnatural amino acids). Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl) alanine, 3-methyl-phenylalanine, O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, Boronophenylalanine, O-propargyltyrosine, L-phosphoserine, phosphonoserine, phosphonotyrosine, p-bromophenylalanine, selenocysteine, p-amino-L-phenylalanine, isopropyl-L-phenylalanine, and azido-lysine (AzK). In some embodiments, the unnatural amino acid is an unnatural analogue of a tyrosine amino acid; an unnatural analogue of a glutamine amino acid; an unnatural analogue of a phenylalanine amino acid; an unnatural analogue of an alanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid; or a combination thereof. In some embodiments, the unnatural amino acid is an amino acid with a photoactivatable cross-linker; a spin-labeled amino acid; a fluorescent amino acid; a metal binding amino acid; a metal-containing amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an α,α-disubstituted amino acid; a β-amino acid; a cyclic amino acid other than proline or histidine, or an aromatic amino acid other than phenylalanine, tyrosine or tryptophan.

Unnatural amino acids include, for example, N-alkyl amino acids in which a natural amino acid described above is N-alkylated, e.g., those modified with lower alkyl groups (for example, of C1 to C5, C1 to C3, and C1) in which the nitrogen forming a peptide bond is branched or not branched. Exemplary N-alkyl amino acids include, e.g., N-ethyl amino acid, N-butyl amino acid, and N-methyl amino acid. Also included are amino acids to which a functional group is further added to the side chain of a natural amino acid or substituted for another functional group (for example, an amino acid having a substitution or an addition in a part such as an arylene group, an alkylene group, or the like of the side chain; an amino acid wherein the arylene group or the alkyl group of the side chain has an increased C-number; an amino acid having a substitution in the aromatic ring of the side chain; a heterocyclic or condensed cyclic amino acid; or the like). Exemplary N-alkyl amino acids further include, e.g., N-alkyllysine and N-methyllysine.

In a non-limiting manner, unnatural amino acids include, but are not limited to N-methyl amino acids, (3-Azetidine)-hAla, (5-Cl) D-Trp, (5-Me) D-Trp, (5-MeO) D-Trp, (6-Me) D-Trp, (7-Me) D-Trp, (D/L) Hly, (R-βBenzyl) D-Trp, (R-βBenzyl) Trp, (R-βCyclopropyl) D-Trp, (R-βCyclopropyl) Trp, (R-βIsobutyl) D-Trp, (R-βIsobutyl) Trp, (R-βIsopropyl) D-Trp, (R-βIsopropyl) Trp, (R-βMe) D-Trp, (R-βMe) Phe, (R-βMe) Trp, (R-βNeopentyl) D-Trp, (R-βNeopentyl) Trp, (R-βPhenyl) D-Trp, (R-βPhenyl) Trp, (R-βPropyl) D-Trp, (R-βPropyl) Trp, (R-βSecbutyl) D-Trp, (R-βSecbutyl) Trp, (S-βBenzyl) D-Trp, (S-βBenzyl) Trp, (S-βCyclopropyl) D-Trp, (S-βCyclopropyl) Trp, (S-βIsobutyl) D-Trp, (S-βIsobutyl) Trp, (S-βIsopropyl) D-Trp, (S-βIsopropyl) Trp, (S-βMe) D-Trp, (S-βMe) Phe, (S-βMe) Trp, (S-βNeopentyl) D-Trp, (S-βNeopentyl) Trp, (S-βPhenyl) D-Trp, (S-βPhenyl) Trp, (S-βPropyl) D-Trp, (S-βPropyl) Trp, (S-βSecbutyl) D-Trp, (S-βSecbutyl) Trp, (βGeminal methyl) D-Trp, (βGeminal methyl) Trp, 2-aza-Trp, 3,3-diPhe, 3,5-diF Tyr, 3MeO-Phe, 3N-Tyr, 3Pal, 4-aminomethyl Phe, 4-aza-Trp, 4MeO-Phe, 4-oxa Lys, 4-oxa NMe-Lys, 4Pal, 5-aza-Trp, 5FY/5F-Tyr, 6-aza-Trp, 7-aza-Trp, Alt, Aph(Hor), azaLys, Aza-Trp, Bzt, Cba, Cba3N, Cbg, Cha, Cha4N, Cha4NH₂, Chg4N, Cpg, D-6F-Trp, D-Aph(Cbm), D-Lys, D-Phg, D-Tpi, D-Trp, D-Tyr, F2CON, F3CON, F4COO, G(cPr), L-DOPA, Lys(diMe), Lys(iPr), Lys(Me), Lys(triMe), Mpd, mTyr, Nlys, NMe-Ala, NMe-Amp, NMe-Asp, NMe-Azidolysine, NMe-Cha4N, NMe-Chg4N, NMe-Cys, NMe-Dab, NMe-Dap, NMe-D-Hcy, NMe-dLys, NMe-D-Trp, NMe-Glu, NMe-Hcy, NMe-hGlu, NMe-hHcy, NMe-hLys, NMe-Hse, NMe-Hse (Se), NMe-Lys, NMe-Nle, NMe-Orn, NMe-Phe, NMe-propargyl alanine, NMe-propargyl glycine, Nva(NH—NH2), Phg, Pic4, PipzaA, Ser(3-azetidine), Ser(Ph), Tme, trans-Hyp, Tyr(OBn), Tyr(Phe), and the like. Note that D-amino acids such as d-alanine may be classified as D-amino acids, but they may also be classified according to the properties of their side chains, and N-methyl amino acids may be classified as N-alkyl amino acids and may also be classified according to the property of the side chain.

In some embodiments, the unnatural amino acids incorporated into the peptides include one or more of: 1) a ketone functional group (as found in para or meta acetyl-phenylalanine) that can be specifically reacted with hydrazines, hydroxylamines and their derivatives (Addition of the keto functional group to the genetic code of Escherichia coli. Wang L, Zhang Z, Brock A, Schultz P G. Proc Natl Acad Sci USA. 2003 Jan. 7; 100 (1): 56-61; Bioorg Med Chem Lett. 2006 Oct. 15; 16 (20): 5356-9. Genetic introduction of a diketone-containing amino acid into proteins. Zeng H, Xie J, Schultz P G), 2) azides (as found in p-azido-phenylalanine) that can be reacted with alkynes via copper catalyzed “click chemistry” or strain promoted (3+2) cycloadditions to form the corresponding triazoles (Addition of p-azido-L-phenylalanine to the genetic code of Escherichia coli. Chin J W, Santoro S W, Martin A B, King D S, Wang L, Schultz P G. J Am Chem Soc. 2002 Aug. 7; 124 (31): 9026-7; Adding amino acids with novel reactivity to the genetic code of Saccharomyces cerevisiae. Deiters A, Cropp T A, Mukherji M, Chin J W, Anderson J C, Schultz P G. J Am Chem Soc. 2003 Oct. 1; 125 (39): 11782-3), or azides that can be reacted with aryl phosphines, via a Staudinger ligation (Selective Staudinger modification of proteins containing p-azidophenylalanine. Tsao M L, Tian F, Schultz P G. Chembiochem. 2005 December; 6 (12): 2147-9), to form the corresponding amides, 3) alkynes that can be reacted with azides to form the corresponding triazole (In vivo incorporation of an alkyne into proteins in Escherichia coli. Deiters A, Schultz P G. Bioorg Med Chem Lett. 2005 Mar. 1; 15 (5): 1521-4), and 4) boronic acids (boronates) than can be specifically reacted with compounds containing more than one appropriately spaced hydroxyl group or undergo palladium mediated coupling with halogenated compounds (Angew Chem Int Ed Engl. 2008; 47 (43): 8220-3. A genetically encoded boronate-containing amino acid., Brustad E, Bushey M L, Lee J W, Groff D, Liu W, Schultz P G).

### Isomers/Stereoisomers

In some embodiments, the compounds described herein exist as geometric isomers. In some embodiments, the compounds described herein possess one or more double bonds. The compounds presented herein include cis, trans, syn, anti, entgegen (E), and zusammen (Z) isomers as well as the corresponding mixtures thereof. In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent.

### Tautomers

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

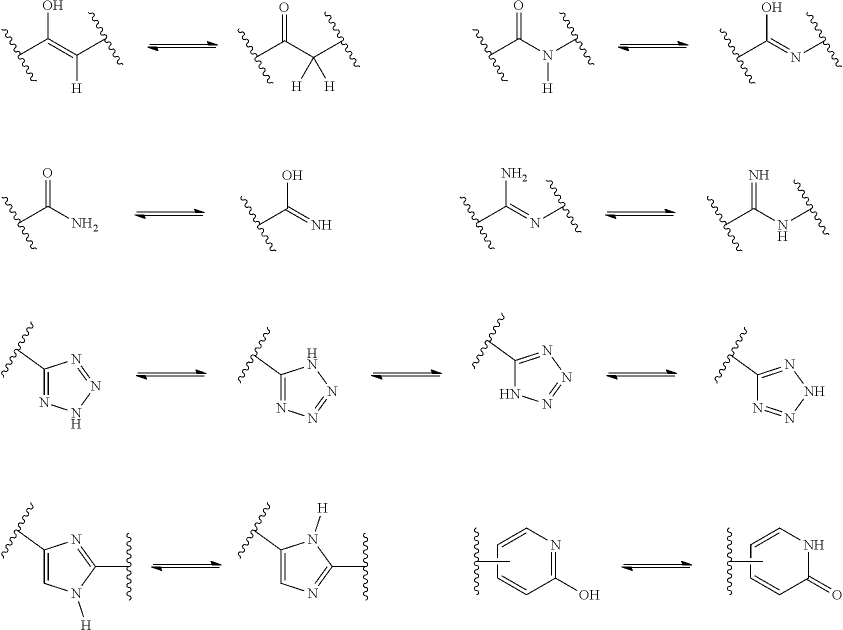

In some instances, the compounds disclosed herein exist in tautomeric forms. The structures of said compounds are illustrated in the one tautomeric form for clarity. The alternative tautomeric forms are expressly included in this disclosure.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ⁵⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their case of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compound or a pharmaceutically acceptable salt, solvate, or stereoisomer thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts.

In some embodiments, the conjugates described herein exist as their pharmaceutically acceptable salts. In some embodiments, the conjugates disclosed herein include their pharmaceutically acceptable salts.

In some embodiments, the conjugates described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the conjugates disclosed herein, or by separately reacting a purified conjugate in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the conjugates described herein can be prepared as pharmaceutically acceptable salts formed by reacting the free base form of the conjugate with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, the conjugates described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaterization of any basic nitrogen-containing groups they contain. In some embodiments, water or oil-soluble or dispersible products are obtained by such quaternization.

### Solvates.

In some embodiments, the conjugates described herein exist as solvates. Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the conjugates described herein can be conveniently prepared or formed during the processes described herein. In addition, the conjugates provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of conjugates of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Preparation of the Conjugates

The disclosure provides methods of preparing and making the conjugates described herein.

The compounds used in the reactions described herein are made according to organic synthesis techniques known to those skilled in this art, starting from commercially available chemicals and/or from compounds described in the chemical literature. “Commercially available chemicals” are obtained from standard commercial sources including ABX advanced biochemical compounds GmbH (Radeberg, Germany), Acros Organics (Pittsburgh, PA), Aldrich Chemical (Milwaukee, WI, including Sigma Chemical and Fluka), Apin Chemicals Ltd. (Milton Park, UK), Avidity Science (U.S.A.), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), ITM (Munich, Germany), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Sigma-Aldrich (U.S.A.), Spectrum Quality Product, Inc. (New Brunswick, NJ), TCI America (Portland, OR), Trans World Chemicals, Inc. (Rockville, MD), VWR (Radnor, PA, USA), Wako Chemicals USA, Inc. (Richmond, VA), and Wuxi-Apptech Inc. (Shanghai, China).

### Pharmaceutical Compositions

The radiopharmaceutical conjugate described herein, including e.g., pharmaceutically acceptable salt or solvate thereof, can be administered per se as a pure chemical or as a component of a pharmaceutically acceptable formulation. In some embodiments, a conjugate described herein is combined with a pharmaceutically suitable or acceptable carrier selected on the basis of a chosen route of administration and standard pharmaceutical practice as described, for example, in Remington: The Science and Practice of Pharmacy (Gennaro, 21^{st} Ed. Mack Pub. Co., Easton, PA (2005)). Provided herein is a pharmaceutical composition comprising at least one conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a herein described conjugate, or a pharmaceutically acceptable salt or solvate thereof, and a pharmaceutically acceptable excipient or carrier.

The conjugates and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intravenous, intrathecal, epidural, and intranasal, etc.

### Method of Treatment

In one aspect, the disclosure provides methods of treating a disease or condition in a subject in need thereof. The methods can comprise administering a radiopharmaceutical conjugate to the subject in need thereof. The methods can provide a therapeutic and/or prophylactic benefit to a subject in need thereof comprising administering a radiopharmaceutical conjugate described herein.

The methods can comprise administering to a subject a radiopharmaceutical conjugate that comprise a therapeutically effective amount of a conjugate or a pharmaceutically acceptable salt or solvate thereof. In some embodiments, the subject has cancer. In some embodiments, the cancer is a solid tumor. In some embodiments, the cancer is an SSTR-associated cancer. In some embodiments, the cancer is an SSTR2-associated cancer. In some embodiments, the cancer is a somatostatin receptor-positive (SSTR+) cancer.

In some embodiments, the cancer is a neuroendocrine cancer, a lymphatic cancer, a pancreatic cancer, a pituitary cancer, a breast cancer, a lung cancer, a stomach cancer, medulloblastoma, or neuroblastoma. In some embodiments, the cancer is a glioblastoma. In some embodiments, the cancer is a neuroendocrine cancer. In some embodiments, the neuroendocrine cancer is a neuroendocrine lung cancer or a neuroendocrine pancreatic cancer. In some embodiments, the neuroendocrine cancer is a Carcinoid tumor in the lungs, gastrointestinal tract or thymus, Pancreatic neuroendocrine tumor (e.g., Gastrinoma, Insulinoma, Glucagonoma, VIPoma) Medullary thyroid carcinoma, Merkel cell carcinoma, Pheochromocytoma of the adrenal gland, Adrenal cancer, Small cell carcinoma (such as in the lungs), or Large cell carcinoid tumor (such as in the lungs). In some embodiments, the cancer is a SSTR2+ lung neuroendocrine tumor. In some embodiments, the cancer is somatostatin receptor-positive (SSTR+) gastroenteropancreatic neuroendocrine tumor (GEP-NET). In some embodiments, the cancer is small cell lung cancer (SCLC). In some embodiments, the cancer is somatostatin receptor expressing (SSTR+) extensive stage small cell lung cancer (ES-SCLC). In some embodiments, the radiopharmaceutical conjugate is administered to the subject in an amount equivalent to about 1 kBq/kg to about 0.2 GBq/kg body weight per dose. In some embodiments, the radiopharmaceutical conjugate is administered to the subject in an amount equivalent to about 5 kBq/kg to about 50,000 kBq/kg body weight per dose. In some embodiments, the radiopharmaceutical conjugate is administered to the subject in an amount equivalent to about 20 kBq/kg to about 5,000 kBq/kg body weight per dose. In some embodiments, the radiopharmaceutical conjugate is administered to the subject in an amount equivalent to about 50 kBq/kg to about 500 kBq/kg body weight per dose. In some embodiments, the radiopharmaceutical conjugate is administered to the subject in an amount equivalent to about 50 kBq/kg to about 200 kBq/kg body weight per dose. In some embodiments, the radiopharmaceutical conjugate is administered to the subject in an amount equivalent to about 60 kBq/kg to about 150 kBq/kg body weight per dose. In some embodiments, the radiopharmaceutical conjugate is administered to the subject at a radioactivity of about 1 μCi to 1,000 μCi. In some embodiments, the radiopharmaceutical conjugate is administered to the subject at a radioactivity of about 10 μCi to 500 μCi. In some embodiments, the radiopharmaceutical conjugate is administered to the subject at a radioactivity of about 100 μCi to 500 μCi. In some embodiments, the radiopharmaceutical conjugate is administered to the subject at a radioactivity of about 100 μCi to 300 μCi. In some embodiments, the radiopharmaceutical conjugate is administered to achieve a cumulative dose in the subject of about 10,000 kBq to about 100,000 kBq. In some embodiments, the radiopharmaceutical conjugate is administered to achieve a cumulative dose in the subject of about 40,000 kBq to about 70,000 kBq.

In some embodiments, provided herein are methods for killing a cell comprising contacting the cell with a conjugate disclosed herein or a pharmaceutically acceptable salt or solvate thereof. In some embodiments, the cell expresses a receptor described herein (e.g., SSTR). In some embodiments, the conjugate or pharmaceutically acceptable salt or solvate thereof binds to a structure on the cell. In some embodiments, the conjugate or pharmaceutically acceptable salt or solvate thereof releases a number of alpha particles by natural radioactive decay. In some embodiments, the conjugate or pharmaceutically acceptable salt or solvate thereof releases a number of beta particles, gamma rays, and/or Auger electrons by natural radioactive decay. The conjugate described herein can kill a cell by radiation. In some embodiments, the conjugate kills the cell directly by radiation. In some embodiments, the radiation creates, in the cell, oxidized bases, abasic sites, single-stranded breaks, double-stranded breaks, DNA crosslink, chromosomal rearrangement, or a combination thereof. The conjugate can kill the cell by inducing double-stranded DNA breaks. The released alpha particles can be sufficient to kill the cell. The released alpha particles can be sufficient to stop cell growth. The conjugate can also kill the cell indirectly via the production of reactive oxygen species (ROS) such as free hydroxyl radicals. In some embodiments, the conjugate kills the cell indirectly by releasing tumor antigens from one or more different cells, which can have vaccine effect. The conjugate can kill the cell by abscopal effect. The cell can be a cancer cell. In some embodiments, the method comprises killing a cell with an alpha-particle emitting radionuclide.

After contacting a cell, the described conjugate can be internalized by the cell. The internalization can be mediated by cell receptors, cell membrane endocytosis, etc. In some embodiments, rapid internalization rate into cancer cells accompanied by a slow externalization rate can offer therapeutic benefit.

In one aspect, the disclosed conjugate or a pharmaceutically acceptable salt or solvate thereof is configured to treat cancer by ablating tumor cells. The conjugate or a pharmaceutically acceptable salt or solvate thereof may not modulate the biology of the tumor cell and/or the surrounding stroma. The conjugate or a pharmaceutically acceptable salt or solvate thereof may not modulate immune cells.

Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer, small cell lung cancer, extensive stage small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subjects to be treated with a radiopharmaceutical conjugate of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, gastroenteropancreatic neuroendocrine tumor, or Merkel cell carcinoma. In some embodiments, a subject or population of subjects to be treated with a radiopharmaceutical conjugate of the present disclosure have a hematological cancer. In some embodiments, the subject has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer and melanoma. The cancer can be an SSTR1-associated cancer. The cancer can be an SSTR2-associated cancer. The cancer can be an SSTR3-associated cancer. The cancer can be an SSTR4-associated cancer. The cancer can be an SSTR5-associated cancer. The cancer can be a neuroendocrine cancer, a lymphatic cancer, a pancreatic cancer, a pituitary cancer, a breast cancer, a stomach cancer, medulloblastoma, or neuroblastoma. The cancer can be a neuroendocrine cancer. The neuroendocrine cancer can be recurrent. The neuroendocrine cancer can be refractory to a radiotherapy that comprises beta-particle emitting radionuclide. The neuroendocrine cancer can be a neuroendocrine lung cancer or a neuroendocrine pancreatic cancer. The neuroendocrine cancer can be a Carcinoid tumor in the lungs, gastrointestinal tract or thymus, Pancreatic neuroendocrine tumor (e.g., Gastrinoma, Insulinoma, Glucagonoma, VIPoma) Medullary thyroid carcinoma, Merkel cell carcinoma, Pheochromocytoma of the adrenal gland, Adrenal cancer, Small cell carcinoma (such as in the lungs), or Large cell carcinoid tumor (such as in the lungs). In some embodiments, the neuroendocrine cancer can be a pancreatic neuroendocrine tumor (e.g. gastroenteropancreatic neuroendocrine tumor (GEP-NET)). In some embodiments, the neuroendocrine cancer can be a gastrointestinal neuroendocrine tumor (GI-NET). In some embodiments, the neuroendocrine cancer can be gastroesophageal pancreatic neuroendocrine tumor.

Gastroenteropancreatic neuroendocrine tumors, or GEP-NETs, are rare tumors with an incidence in the U.S. of 5.45 cases per 100,000. Despite this low incidence, many GEP-NETs follow a more indolent disease course than other epithelial malignancies and thus the prevalence of GEP-NETs in the U.S. is approximately 100,000. GEP-NET tumors can be aggressive and resistant to therapy and based on the Surveillance, Epidemiology, and End Results database, metastatic disease is present at diagnosis in 40-76% of cases. Depending on their morphology and proliferative activity, GEP-NETs can be classified as well-differentiated tumors or poorly differentiated carcinomas. Well-differentiated GEP-NETs can include low-grade (Grade 1, defined as tumors with a mitotic rate of 0-1 per 10 high power field, or HPF, or a Ki67 index from 0-2%) and intermediate-grade tumors (Grade 2, defined as tumors with a mitotic rate from 2-20 per 10 HPF or a Ki67 index from 3-20%), whereas poorly differentiated GEP-NETs can be high-grade (Grade 3, with a mitotic rate greater than 20 per 10 HPF or a Ki67 index greater than 20%).

In some embodiments, the site of primary NETs in the digestive tract is the rectum, small intestine, pancreas, stomach, colon, and/or appendix. In some embodiments, the GEP-NET is categorized as hormonally functional (associated with signs and symptoms consistent with excess hormone secretion). In some embodiments, the GEP-NET is categorized as non-functional tumors, with clinical features and aggressiveness depending on the primary tumor site. In some embodiments, the cancer is pancreatic neuroendocrine tumors (pNETs).

In some embodiments, provided herein are methods and conjugates for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, small cell lung cancer, extensive stage small cell lung cancer, hepatocellular cancer, colorectal cancer, gastric adenocarcinoma, pancreatic neuroendocrine tumor (e.g. gastroenteropancreatic neuroendocrine tumor), melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, small cell lung cancer, extensive stage small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, pancreatic neuroendocrine tumors, gastroenteropancreatic neuroendocrine tumors, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, the cancer is a hypoxic tumor. In some embodiments, the cancer is GEP-NET. In some embodiments, the cancer is small cell lung cancer. In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is lung cancer. In some embodiments, a cancer to be treated by the methods of treatment of the present disclosure is small cell lung cancer (SCLC). In some embodiments, a cancer to be treated by the methods of treatment of the present disclosure is extensive stage small cell lung cancer (ES-SCLC). In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer. In some embodiments, a cancer to be treated by the methods of treatment of the present disclosure is a pancreatic neuroendocrine tumor. In some embodiments, a cancer to be treated by the methods of treatment of the present disclosure is a gastroenteropancreatic neuroendocrine tumor. In some embodiments, a cancer to be treated by the methods of treatment of the present disclosure is GEP-NET. In some embodiments, a cancer to be treated by the methods of treatment of the present disclosure is gastroesophageal pancreatic neuroendocrine tumor.

In addition to the methods of treatment described above, the radiopharmaceutical conjugates described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes such as the isotopes in Table 4 labeled “Dx”. Accordingly, the conjugate can be administered as a companion diagnostic.

In one aspect, provided herein are methods for diagnosing a patient harboring a somatostatin receptor (SSTR) expressing cancer or tumor comprising administering to the patient a radiopharmaceutical described herein, or a pharmaceutically acceptable salt or solvate thereof, or a pharmaceutical composition comprising the same. In one aspect, provided herein are methods for imaging an SSTR expressing cancer or tumor comprising administering to the patient a radiopharmaceutical described herein, or a pharmaceutically acceptable salt or solvate thereof, or a pharmaceutical composition comprising the same. In some embodiments, the method further comprises selecting or confirming that a tumor in the patient expresses SSTR. In some embodiments, the SSTR expressing cancer is an SSTR2 expressing cancer. In some embodiments, the method further comprises measuring the concentration of the radiopharmaceutical accumulated in the patient. In some embodiments, the method further comprises measuring the amount of radiation emitted from the radionuclide. In some embodiments, the method further comprises analyzing the elimination or clearance profile of the radiopharmaceutical in the patient. In some embodiments, the method further comprises measuring an elimination half-life of the radiopharmaceutical in the patient. In some embodiments, the method further comprises analyzing the clearance profile of the radiopharmaceutical in the patient. In some embodiments, the method of imaging or diagnosing cancer comprises administering a radiopharmaceutical that comprises a radionuclide of Table 4 labeled “Dx”, such as ⁶⁸Ga. For example, radiopharmaceuticals of the present disclosure can be administered for patient selection purposes, such as to confirm the tumor has the appropriate expression of the SSTR target (e.g., SSTR2). As another example, radiopharmaceuticals of the present disclosure can be administered to a patient so that the patient's care team can make sure the radiopharmaceutical is cleared from the body in a suitable timeframe so that undesired irradiation of other tissues is minimized.

In some embodiments, a method described herein comprises administering to a patient two radiopharmaceuticals of the present disclosure. In some embodiments, the two radiopharmaceuticals can have the same targeting ligand. In some embodiments, a method described herein comprises administering (i) a radiopharmaceutical of the present disclosure that comprises a radionuclide of Table 4 labeled “Dx”, and followed by (i) a radiopharmaceutical of the present disclosure that comprises a radionuclide of Table 4 labeled “Tx”. In some embodiments, the method comprises administering a ⁶⁸Ga labeled conjugate described herein followed by the administering of an ²²⁵Ac labeled conjugate described herein.

The subject can be 4 to 120 years old. The subject can be 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65, 10 to 75, 15 to 18, 15 to 25, 15 to 35, 15 to 45, 15 to 55, 15 to 65, 15 to 75, 18 to 25, 18 to 35, 18 to 45, 18 to 55, 18 to 65, 18 to 75, 25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. The subject can be at least 5, 10, 15, 18, 25, 35, 45, 55, or 65 years old. The subject can be at most 10, 15, 18, 25, 35, 45, 55, 65, or 75 years old.

In some embodiments, the subject has been diagnosed with a SSTR expressing lesion, neoplasm, cancer, or tumor. In some embodiments, the SSTR expressing lesion, neoplasm, cancer, or tumor is characterized quantifying the amount of SSTR expression and calculating an H-score. In some embodiments, the H-score is derived by a) staining a tissue biopsy for SSTR expression (e.g., SSTR2), b) assigning a cell staining intensity of 0, 1, 2, or 3, c) and multiplying the percentage of cells by their staining intensity level. The H-score has a range of 0 to 300. In some embodiments, the SSTR expressing lesion, neoplasm, cancer, or tumor has an H-score of greater than 10. In some embodiments, the SSTR expressing lesion, neoplasm, cancer, or tumor has an H-score of greater than 100. In some embodiments, the SSTR expressing lesion, neoplasm, cancer, or tumor has an H-score of 11 to 300. In some embodiments, the SSTR expressing lesion, neoplasm, cancer, or tumor has an H-score of greater than 200. In some embodiments, the H-score is between 11 and 100. In some embodiments, the H-score is between 50 and 150. In some embodiments, the H-score is between 100 and 200. In some embodiments, the H-score is between 150 and 250. In some embodiments, the H-score is between 200 and 300.

Accordingly, provided herein is a method of treating cancer in a subject in need thereof, the method comprising determining an H-score for a lesion, neoplasm, cancer, or tumor tissue sample of the subject and administering to the subject a radiopharmaceutical conjugate described herein. In one aspect, provided herein is a method of treating a subject having an SSTR+ cancer, comprising administering to the subject a radiopharmaceutical conjugate described herein. In some embodiments, a cancer sample of the subject has been subjected to a determination of an H-score prior to the administration of a conjugate disclosed herein. In some embodiments, a cancer sample of the subject has been subjected to a determination of an H-score, and wherein the H-score is at least 11. In some embodiments, the method comprises determining an H-score for a tumor cancer of the subject. In some embodiments, the H-score is 0 to 300. In some embodiments, the H-score is greater than 10. In some embodiments, the H-score is greater than 50. In some embodiments, the H-score is greater than 100. In some embodiments, the H-score is greater than 150. In some embodiments, the H-score is greater than 100. In some embodiments, the H-score is greater than 200. In some embodiments, the H-score is about 120 to 300. n some embodiments, the H-score is about 40 to 300. In some embodiments, the H-score is about 50 to 300. In some embodiments, the H-score is about 100-300. In some embodiments, the H-score is 200-300. An H-score can be calculated according to the method described in WO2023191839A2.

In one aspect, described herein is a method of treating an SSTR+ cancer in a subject in need thereof, comprising administering to the subject a herein-disclosed radiopharmaceutical conjugate, wherein an H-score of a cancer sample of the subject is at least 11. In one aspect, described herein is a method of treating a disease in a subject in need thereof, comprising (a) determining a level of SSTR expression in a sample of the subject; and (b) administering to the subject a herein-disclosed radiopharmaceutical conjugate. In some embodiments, the level of SSTR expression in a sample is determined by calculating an H-Score. In some embodiments, the H-Score is between 11 and 300. In some embodiments, the H-Score is between 11 and 200. In some embodiments, the H-Score is between 11 and 100. In some embodiments, the H-Score is between 101 and 300. In some embodiments, the H-Score is between 101 and 201. In some embodiments, the H-Score is between 201 and 300. In some embodiments, the radiopharmaceutical conjugate comprises the structure of Formula (IV1), (IV2), (IV3), (IV4), (IV5), or (IV6).

In some embodiments, the radiopharmaceutical conjugates described herein can be administered alone or in combination with one or more additional therapeutic agents. For example, the combination therapy can include a composition comprising a radiopharmaceutical conjugate described herein co-formulated with, and/or co-administered with, one or more additional therapeutic agents, e.g., one or more anti-cancer agents, e.g., cytotoxic or cytostatic agents, immune checkpoint inhibitors, hormone treatment, vaccines, and/or immunotherapies. In some embodiments, the radiopharmaceutical conjugate is administered in combination with other therapeutic treatment modalities, including surgery, cryosurgery, and/or chemotherapy. Such combination therapies may advantageously utilize lower dosages of the administered therapeutic agents, thus avoiding possible toxicities or complications associated with the various monotherapies. In some embodiments, the additional anti-cancer agent is a local or regional cancer treatment. In some embodiments, the local or regional cancer treatment comprises microwave ablation. In some embodiments, the local or regional cancer treatment comprises radiofrequency ablation (RFA). In some embodiments, the local or regional cancer treatment comprises ultrasound ablation. In some embodiments, the local or regional cancer treatment comprises magnetic resonance guided focused ultrasound ablation.

In some embodiments, the local or regional cancer treatment comprises hyperthermia treatment. Different types of techniques can be used to create heat for the hyperthermia treatment. For example, the techniques can include microwaves or probes that make energy from microwaves, radio waves (or radiofrequency), lasers, ultrasound (e.g., high intensity ultrasound), heating fluids such as blood or chemotherapy drugs and putting them into the body (or perfusion), placing the entire body in a heated chamber or hot water bath or wrapping with heated blankets, or a combination thereof.

In some embodiments, the local or regional cancer treatment comprises cryoablation.

When administered in combination, two (or more) different treatments can be delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In some embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Co-administration of amino acids with a radiopharmaceutical described herein may reduce kidney update of the radiopharmaceutical. Reducing kidney uptake and/or increasing clearance of the radiopharmaceutical is a desired property in radiopharmaceutical therapeutics. In some embodiments, a radiopharmaceutical conjugate described herein is concurrently administered with an intravenous infusion of one or more amino acids. In some embodiments, a radiopharmaceutical conjugate described herein is administered after an intravenous infusion of one or more amino acids. In some embodiments, the infusion of the one or more amino acids is administered at least 30 minutes prior to administering a radiopharmaceutical conjugate described herein. In some embodiments, the infusion of the one or more amino acids is administered at least 6 hours, 3 hours, 1 hour, 30 minutes, 20 minutes, 10 minutes, or 5 minutes prior to administering a radiopharmaceutical conjugate described herein. In some embodiments, the infusion of the one or more amino acids is administered 10 to 60 minutes prior to administering a radiopharmaceutical conjugate described herein.

The radiopharmaceutical conjugates of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. The term parenteral as used herein includes e.g., subcutaneous, intravenous, intramuscular, intrasternal, intraperitoneal, and infusion techniques. The term parenteral also includes injections, into the eye or ocular, intravitreal, intrabuccal, transdermal, intranasal, into the brain, including intracranial and intradural, into the joints, including ankles, knees, hips, shoulders, elbows, wrists, and the like, and in suppository form. The radiopharmaceutical conjugates can be administered orally. The radiopharmaceutical conjugates can be administered by systemic administration. The radiopharmaceutical conjugates can be administered parenterally. The radiopharmaceutical conjugates can be administered intravenously. The radiopharmaceutical conjugates can be administered locally at a targeted site.

The radiopharmaceutical conjugates described herein can be administered via parenteral injection as liquid solution.

The radiopharmaceutical conjugates can be administered in a manner appropriate to the disease to be treated. An appropriate dose and a suitable duration and frequency of administration can be determined by such factors as the condition of the subject, the type and severity of the subject's disease, the particular form of the active ingredient, and the method of administration. In some embodiments, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the subject. In some embodiments, the radiopharmaceutical conjugate can be administered as part of a first-line therapy. In some embodiments, the radiopharmaceutical conjugate can be administered as part of a first-line therapy with existing standard of care, for example, immune checkpoint blockers and DNA damaging agents.

In some embodiments, the compositions comprising radiopharmaceutical conjugates described herein can comprise conjugates that exist as solvates. This disclosure provides for methods of treating diseases by administering such solvates. This disclosure further provides for methods of treating diseases by administering such solvates as radiopharmaceutical compositions.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### A: Synthesis of the Conjugates

Unless otherwise stated in the present specification, the following abbreviations are used according to the following meanings:
Alloc allyloxycarbonyl
aq. aqueous
Biotin-OSu biotin N-hydroxysuccinimide ester (CAS 35013-72-0)
Boc tert-butyloxycarbonyl
ClAcOH chloroacetic acid
ClAcOSu N-succinimidyl 2-chloroacetate (CAS 27243-15-8)
DCM dichloromethane (CAS 75-09-2)
DIC N,N′-diisopropylcarbodiimide (CAS 693-13-0)
DIPEA, DIEA N,N-diisopropylethylamine (CAS 7087-68-5)
DMEM Dulbecco's Modified Eagle Medium
DMF N,N-dimethylformamide (CAS 68-12-2)
DODT 2,2′-(ethylenedioxy)diethanethiol (CAS 14970-87-7)
EDCI-HCl N-(3-dimethylaminopropyl)-N′-ethylcarbodiimide hydrochloride (CAS 25952-53-8)
EDTA ethylenediaminetetraacetic acid
EMEM Eagle's minimal essential medium
eq equivalent
Et ethyl
Et₃N, TEA triethylamine (CAS 121-44-8)
FBS fetal bovine serum
Fmoc 9-fluorenylmethoxycarbonyl
hr hour
HATU 1-[bis(dimethylamino)methylene]-1H-1,2,3-triazolo[4,5-b]pyridinium-3-oxide hexafluorophosphate (CAS 148893-10-1)
HOSu N-hydroxysuccinimide (CAS 6066-82-6)
iPrOH, IPA isopropanol
M molar
min minutes
NHS N-hydroxysuccinimide (CAS 6066-82-6)
NMP N-methylpyrrolidone (CAS 872-50-4)
Pd(PPh₃)₄ tetrakis (triphenylphosphine) palladium (0) (CAS 14221-01-3)
PG protecting group
Ph phenyl
RP reverse phase
rpm rotations per minute
rt room temperature
SAPE Streptavidin, R-Phycoerythrin conjugate
SPPS solid phase peptide synthesis
Su succinimidyl
SulfoCy5-sulfo Cyanine5 (CAS 2791287-13-1)
SulfoCy5-OSu, (CAS 146368-14-1)
SulfoCy5-NHS see SulfoCy5-OSu
tert tertiary
TFA trifluoroacetic acid (CAS 76-05-1)
TIS triisopropylsilane (CAS 6485-79-6)
tr retention time
Trt trityl
PyAOP (7-azabenzotriazol-1-yloxy)trispyrrolidinophosphonium hexafluorophosphate (CAS 156311-83-0)
MeCN acetonitrile
AcOH acetic acid
Et₂O diethyl ether
AA amino acid
Me methyl
HFIP 1,1,1,3,3,3-hexafluoro-2-propanol (CAS 920-66-1)
PhSiH₃ phenylsilane (CAS 694-53-1)
tBu tertiary butyl
DMSO dimethylsulfoxide
Mpe 3-Methyl-pent-3-yl

### Example A1. Analytical Conditions

Solid phase peptide synthesis (SPPS) was performed in a standard manual reaction vessel under nitrogen. 2-CTC resin was purchased from Sunresin New Materials Co. (China). Fmoc protected amino acids were purchased from GL Biochem (China). HBTU and HATU were purchased from Highfine Biotech Co. (China). Piperidine was purchased from Damao Chemical Reagent Factory (China). The peptides and their derivatives were purified on a Gilson GX-281 preparative HPLC system using reverse-phase C18 columns (Gemini, 5 μm, 110 Å+ luna, 10 μm, 100 Å) at 30° C. HPLC solvents consisted of H₂O containing 0.075% trifluoroacetic acid (mobile phase A) and acetonitrile (mobile phase B).

High performance liquid chromatography (HPLC) analyses were performed on an Agilent 1260 series equipped with a binary pump G7112A, micro vacuum degasser, standard autosampler ALS G7129A, thermostated column compartment TCC G7116A, variable wavelength detector VWD G7114A, and data were analyzed by OpenLab CDS 2.2 network workstation software from Agilent Technologies. HPLC solvents consisted of H₂O containing 0.1% trifluoroacetic acid (mobile phase A) and acetonitrile containing 0.075% trifluoroacetic acid (mobile phase B). Conditions: a Phenomenex Gemini-NX C-18 (5 μm, 110 Å, 4.6×250 mm) column was used with a flow rate of 1.0 mL/min.

LC-MS analyses were carried out on an Agilent 1200 series coupled to an Agilent MSD G6125C, equipped with a binary pump G7112A, micro vacuum degasser, standard autosampler ALS G7129A, thermostated column compartment TCC G7116A, variable wavelength detector VWD G7114A, and data were analyzed by OpenLab CDS 2.3 standalone workstation software from Agilent Technologies. HPLC solvents consisted of H₂O containing 0.1% trifluoroacetic acid (mobile phase A) and acetonitrile containing 0.075% trifluoroacetic acid (mobile phase B). Conditions: a Waters Xbridge C-18 (3.5 μm, 3.1×30 mm) column was used with a flow rate of 1.2 mL/min.

### Example A2. Synthesis of Intermediate (Int) A

### Synthesis of Int A

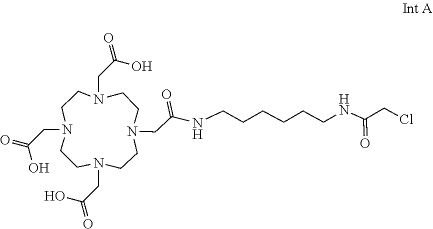 [SMILES: O=C(O)CN1CCN(CC(=O)O)CCN(CC(=O)NCCCCCCNC(=O)CCl)CCN(CC(=O)O)CC1]

2,2′,2″-(10-(2-((6-(2-Chloroacetamido) hexyl)amino)-2-oxoethyl)-1,4,7,10-tetraazacyclododecane-1,4,7-triyl)triacetic acid (Int A). To the swollen 2-CTC resin was added Fmoc-1,6-diaminohexane hydrochloride (1 mmol) and DIEA (4 mmol) in DCM (5 mL). The mixture was agitated for 15 h under nitrogen. MeOH (1 mL) was then added. The resulting mixture was agitated for 30 min. After the reaction solution was removed through filtration, the resin was washed three times with DMF (10 mL). The Fmoc protecting group was removed via 15 min agitation with 20% piperidine in DMF followed by filtration and washing.

To the peptidyl-resin was added DOTA (3tBu) (3 equiv.), DIEA (6 equiv.) and HBTU (3 equiv.) in DMF (5 mL). The mixture was agitated for 1 h under nitrogen. After the reaction solution was removed through filtration, the resin was washed three times with DMF (10 mL).

After the resin was washed three times with MeOH and dried under vacuum, the peptidyl-resin was treated with 10 mL TFA cleavage cocktail for 2.5 hour, cold isopropyl ether was added. The crude peptide was collected through centrifugation and dried under vacuum.

The crude peptide was dissolved in H₂O (5 mL), then the solution was s adjusted to pH=8 with saturated Na₂CO₃ aqueous solution and 2,5-dioxopyrrolidin-1-yl 2-chloroacetate (1.5 equiv.) was added. The mixture was stirred at 25° C. for 10 min and keep pH=7-8. After the reaction was completed, the mixture was purified by preparative HPLC to afford Int A (270 mg, 95% purity) as a white solid.

### Example A3. Synthesis of Intermediate (Int) B

### Synthesis of Int B

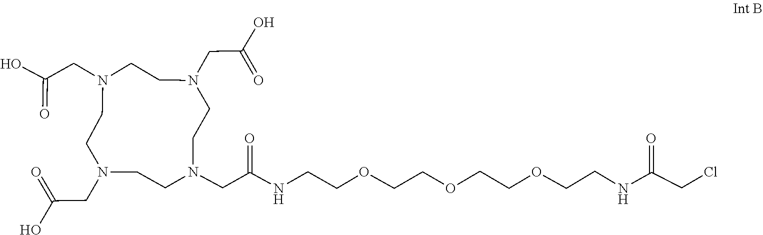 [SMILES: O=C(O)CN1CCN(CC(=O)O)CCN(CC(=O)NCCOCCOCCOCCNC(=O)CCl)CCN(CC(=O)O)CC1]

2,2′,2″-(10-(17-Chloro-2,16-dioxo-6,9,12-trioxa-3,15-diazaheptadecyl)-1,4,7,10-tetraazacyclododecane-1,4,7-triyl)triacetic acid (Int B). To the swollen 2-CTC resin were added Fmoc-PEG3-CH₂CH₂NH₂·HCl (1 mmol) and DIEA (4 mmol) in DCM (5 mL). The mixture was agitated for 15 h under nitrogen. MeOH (1 mL) was then added. The resulting mixture was agitated for 30 min. After the reaction solution was removed through filtration, the resin was washed three times with DMF (10 mL). The Fmoc protecting group was removed via 15 min agitation with 20% piperidine in DMF followed by filtration and washing.

To the peptidyl-resin was added DOTA (3tBu) (3 equiv.), DIEA (6 equiv.) and HBTU (3 equiv.) in DMF (5 mL). The mixture was agitated for 1 h under nitrogen. After the reaction solution was removed through filtration, the resin was washed three times with DMF (10 mL).

After the resin was washed three times with MeOH and dried under vacuum, the peptidyl-resin was treated with 10 mL TFA cleavage cocktail for 2.5 hour, cold isopropyl ether was added. The crude peptide (480 mg) was collected through centrifugation and dried under vacuum.

The crude peptide was dissolved in H₂O (5 mL), then the solution was s adjusted to pH=8 with saturated Na₂CO₃ aqueous solution and 2,5-dioxopyrrolidin-1-yl 2-chloroacetate (1.5 equiv.) was added. The mixture was stirred at 25° C. for 10 min and keep pH=7-8. After the reaction was completed, the mixture was purified by preparative HPLC to afford Int B (292 mg, 95% purity) as a white solid.

### Example A4. Synthesis of Intermediate (Int) C

### Synthesis of Int C

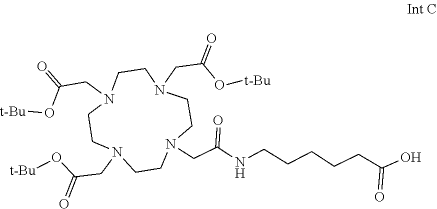 [SMILES: CC(C)(C)OC(=O)CN1CCN(CC(=O)NCCCCCC(=O)O)CCN(CC(=O)OC(C)(C)C)CCN(CC(=O)OC(C)(C)C)CC1]

6-(2-(4,7,10-Tris (2-(tert-butoxy)-2-oxoethyl)-1,4,7,10-tetraazacyclododecan-1-yl)acetamido) hexanoic acid (Int C). To the swollen 2-CTC resin was added 6-(Fmoc-amino) hexanoic acid (0.50 mmol) and DIEA (2 mmol) in DCM (5 mL). The mixture was agitated for 2 h under nitrogen. MeOH (1 mL) was then added. The resulting mixture was agitated for 30 min. After the reaction solution was removed through filtration, the resin was washed three times with DMF (10 mL). The Fmoc protecting group was removed via 15 min agitation with 20% piperidine in DMF followed by filtration and washing.

To the peptidyl-resin was added DOTA (3tBu) (3 equiv.), DIEA (6 equiv.) and HBTU (3 equiv.) in DMF (5 mL). The mixture was agitated for 1 h under nitrogen. After the reaction solution was removed through filtration, the resin was washed three times with DMF (10 mL).

After the resin was washed three times with MeOH and dried under vacuum, the resin was treated with 20% HFIP/DCM for 15 min 3 times. The mixture was concentrated to dryness and then lyophilized to give Int C (300 mg).

### Example A5. Synthesis of Intermediate (Int) D

### Synthesis of Int D

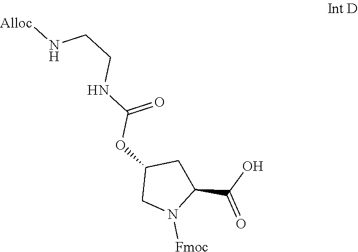

(2S,4R)-1-(((9H-Fluoren-9-yl) methoxy) carbonyl)-4-(((2-(((allyloxy) carbonyl)amino)ethyl) carbamoyl)oxy) pyrrolidine-2-carboxylic acid (Int D). Fmoc-Hyp(Bom)-OH (1.0 g) was treated with 50% TFA/DCM for 30 min, then concentrated to dryness. The material was then dissolved in DMF (10 mL) and Alloc-OSu (1.2 equiv.) and DIPEA (3 equiv.) were added, the mixture was stirred at 25° C. for 1 hour. Then the mixture was purified by preparative HPLC to afford Int D (535 mg, 95% purity) as a white solid.

### Example A6. Synthesis of Intermediate (Int) E

### Synthesis of Int E

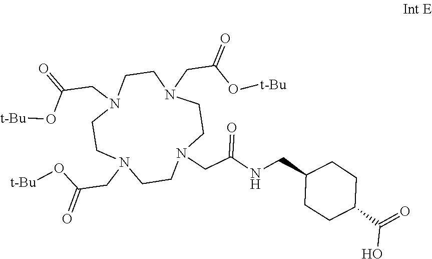 [SMILES: CC(C)(C)OC(=O)CN1CCN(CC(=O)NC[C@H]2CC[C@H](C(=O)O)CC2)CCN(CC(=O)OC(C)(C)C)CCN(CC(=O)OC(C)(C)C)CC1]

(1r,4r)-4-((2-(4,7,10-Tris (2-(tert-butoxy)-2-oxoethyl)-1,4,7,10-tetraazacyclododecan-1-yl)acetamido)methyl)cyclohexane-1-carboxylic acid (Int E). To the swollen 2-CTC resin was added Fmoc-tranexamic acid (2.5 mmol) and DIEA (10 mmol) in DCM (25 mL). The mixture was agitated for 4 h under nitrogen. MeOH (5 mL) was then added. The resulting mixture was agitated for 30 min. After the reaction solution was removed through filtration, the resin was washed three times with DMF. The Fmoc protecting group was removed via 20 min agitation with 20% piperidine in DMF followed by filtration and washing with DMF.

To the peptidyl-resin was added DOTA (3tBu) (3 equiv.), DIEA (6 equiv.) and HATU (3 equiv.) in DMF (25 mL). The mixture was agitated for 18 h under nitrogen. After the reaction solution was removed through filtration, the resin was washed three times with DMF.

After the resin was washed three times with MeOH and dried under vacuum, the resin was treated with 20% HFIP/DCM for 15 min 3 times. The mixture was concentrated to dryness and then lyophilized to give Int E (1.7 g).

### Example A7. Synthesis of Intermediate (Int) F

### Synthesis of Int F

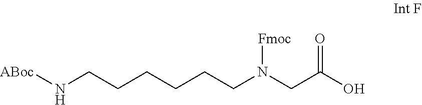

N-(((9H-Fluoren-9-yl) methoxy) carbonyl)-N-(6-(((allyloxy) carbonyl)amino) hexyl)glycine (Int F). To the swollen 2-CTC resin was added BrAc (1 mmol) and DIEA (4 mmol) in DCM (5 mL). The mixture was agitated for 2 h under nitrogen. MeOH (1 mL) was then added. The resulting mixture was agitated for 30 min. After the reaction solution was removed through filtration, the resin was washed three times with DMF (10 mL).

To the peptidyl-resin was added tert-butyl (6-aminohexyl) carbamate (2 equiv.) and DIEA (4 equiv.) in DMF (5 mL). The mixture was agitated for 1 h under nitrogen. After the reaction solution was removed through filtration, the resin was washed three times with DMF (10 mL). To the peptidyl-resin was added Fmoc-C1 (2 equiv.) and DIEA (4 equiv.) in DMF (5 mL). The mixture was agitated for 1 h under nitrogen. After the reaction solution was removed through filtration, the resin was washed three times with DMF (10 mL).

After the resin was washed three times with MeOH and dried under vacuum, the resin was treated with 50% TFA/DCM for 30 min. The mixture was concentrated to dryness. The crude residue was dissolved in DMF (5 mL) and Alloc-OSu (1.2 equiv.) and DIPEA (3 equiv.) were added, and the mixture was stirred at 25° C. for 1 hour. Then the mixture was concentrated and purified by preparative HPLC to afford Int F (292 mg) as a white solid.

### Example A8. Synthesis of Intermediate (Int) 5 by Method 1

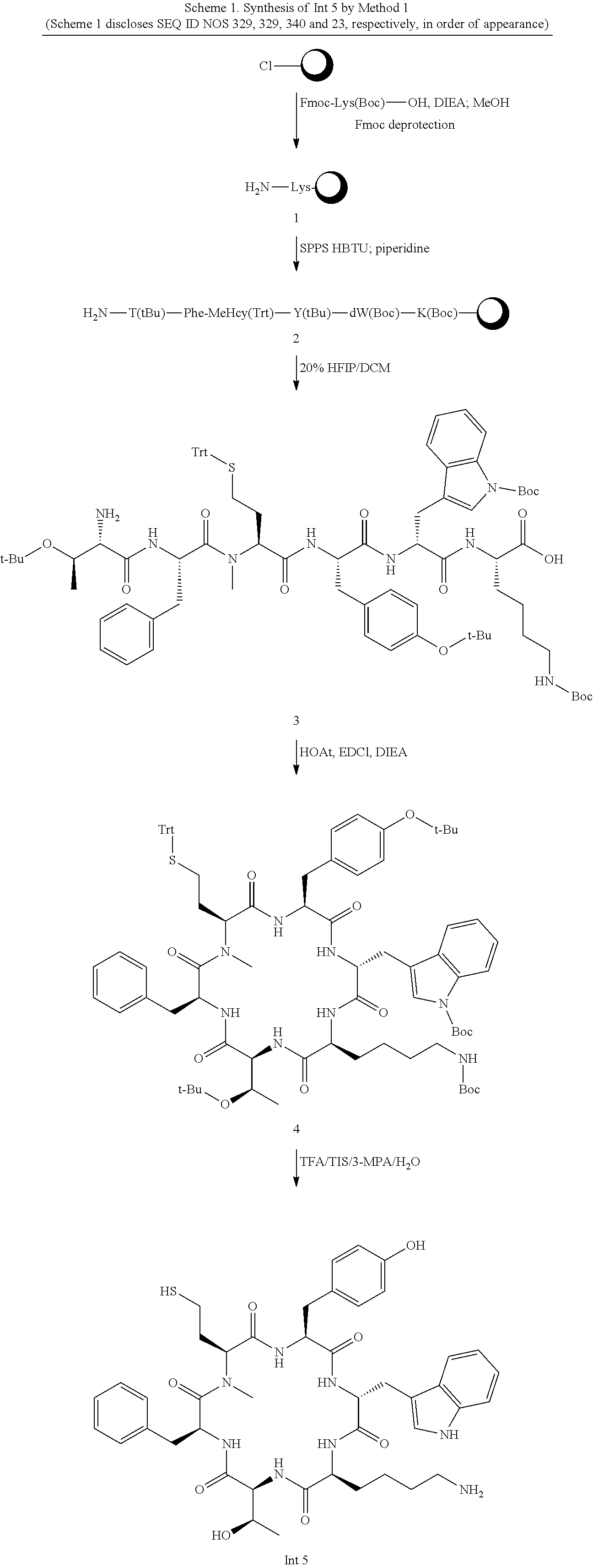

### Method 1:

To the swollen 2-CTC resin was added Fmoc-Lys(Boc)-OH (1 equiv.) and DIEA (4 equiv.) in DCM (10 mL). The mixture was agitated for 2 h under nitrogen. MeOH (1 mL) was then added. The resulting mixture was agitated for 30 min. After the reaction solution was removed through filtration, the resin was washed three times with DMF (15 mL). The Fmoc protecting group was removed via 15 min agitation with 20% piperidine in DMF followed by filtration and washing.

To the peptidyl-resin 1 were added Fmoc-D-Trp(Boc)-OH (3 equiv.), DIEA (6 equiv.) and HBTU (3 equiv.) in DMF (10 mL). The mixture was agitated for 1 h under nitrogen. After the reaction solution was removed through filtration, the resin was washed three times with DMF (15 mL). The Fmoc protecting group was removed via 20 min agitation with 20% piperidine in DMF followed by filtration and washing.

Subsequent amino acids were coupled using Fmoc-protected amino acid (3 equiv.), HBTU (3 equiv.) and DIEA (6 equiv.) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

The peptidyl-resin 2 was washed three times with MeOH and dried under vacuum, the resin was treated with 20% HFIP/DCM for 15 min 3 times. The mixture was concentrated to dryness and then lyophilized to give Int 3

The Int 3 was dissolved in DCM (1 mM) together with EDCI (1.5 equiv.), HOAt (1.5 equiv.) and DIEA (3 equiv.). The mixture was stirred at 20° C. for 13 h. After the reaction was completed, the mixture was concentrated to dryness to give Int 4.

The peptide was treated with 15 mL of TFA cleavage cocktail for 1 hour, then cold isopropyl ether was added. The crude peptide was collected through centrifugation and dried under vacuum. The crude product was purified by preparative HPLC to afford Int 5 (93% purity) as a white solid.

### Example A9. Synthesis of RB-0003149 from Int 5 by Method 1

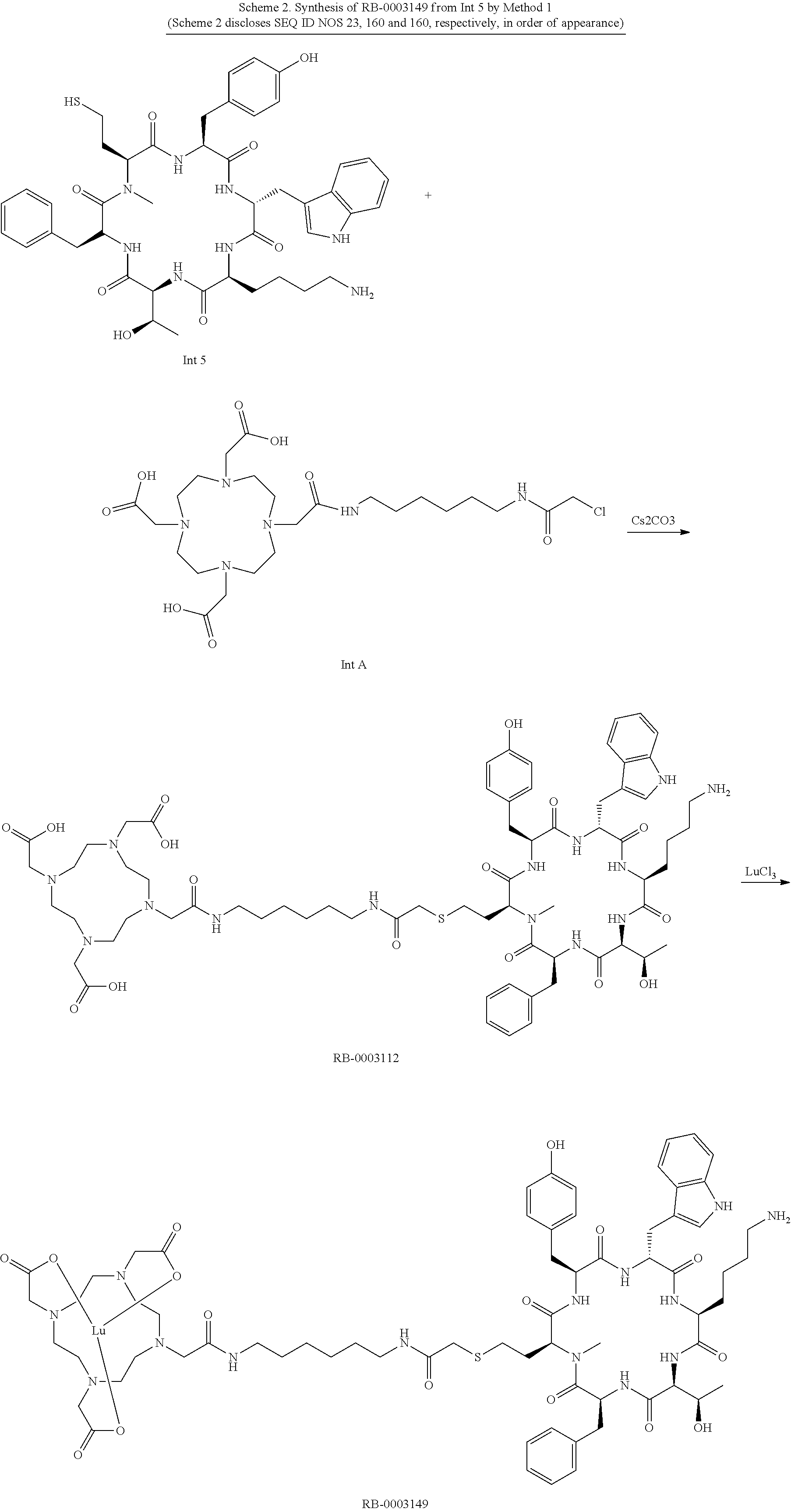

The Int 5 (1 equiv.) was dissolved in 30% ACN/H2O (30 mL) and Int A (1.5 equiv.) was added at 25° C. The solution was adjusted to pH=8˜9 with saturated Cs₂CO₃ aqueous solution and stirred at 25° C. for 3 hour. Then I mol/L HCl was adjusted to pH=6˜7, the mixture was lyophilized to give the crude powder. The crude product was purified by preparative HPLC to afford RB-0003112 as a white solid.

Following the general protocol for cold labeling of empty-chelator conjugates with Lu, La and Ga metals (See Example A16)—A solution of RB-0003112 (1 equiv.) in 1M Na₂CO₃ aqueous buffer adjusted to pH=5˜6 was added LuCl₃ (5 equiv.). The resulting mixture was stirred at 40° C. for 1 h. After filtration, the crude product was purified by preparative HPLC to afford RB-0003149 as a white solid.

Additional peptide analogues were prepared similarly using this modular synthetic approach outlined in Method 1. The amino acids, fragments, linker, and chelator used in this method vary depending on the peptide sequence. In some instances, the X1 amino acid has a side chain with a terminal thiol, hydroxyl functionality. In some instances, the chelator is a chelator described herein. In some embodiments, the chelator is NOTA, DOTA, or NODAGA. Additional peptides prepared according to Method 1 are shown in Table 5.

TABLE 5

| | | Exact | Mass | Method |
|---|---|---|---|---|
| Conjugate | | mass | observed | of |
| ID | Structure | (g/mol) | (g/mol) | Synthesis |
| RB- 0002674 (147) | 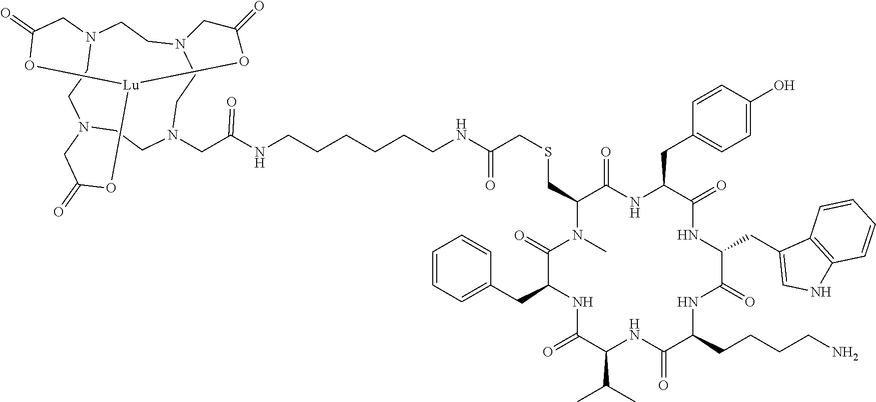 | 1554.623 | ESI-MS m/z: [M/2 + H]+ = 778.6 | Method 1; Purity: 95.43% |
| RB- 0002676 (148) | 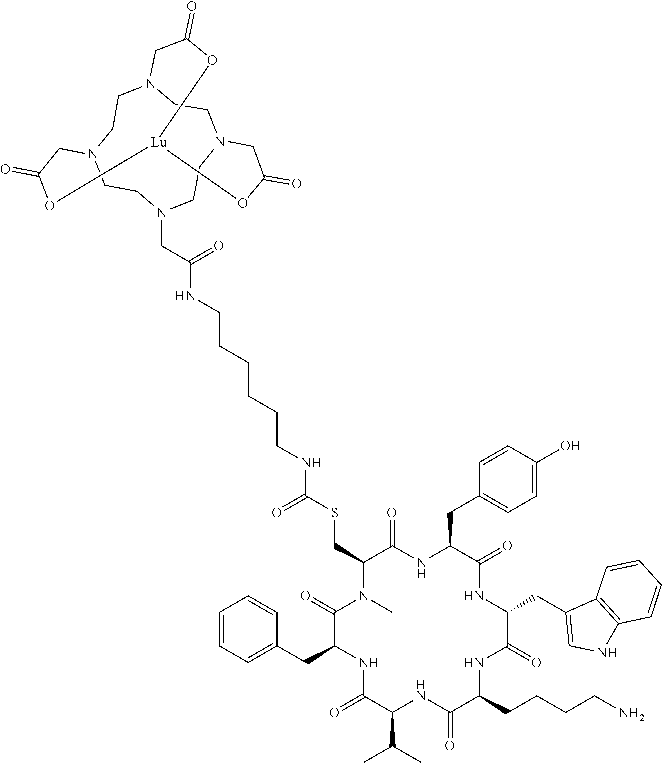 | 1568.6386 | ESI-MS m/z: [M + H]+ = 1569.9 | Method 1; Purity: 96.28% |
| RB- 0002946 (151) | 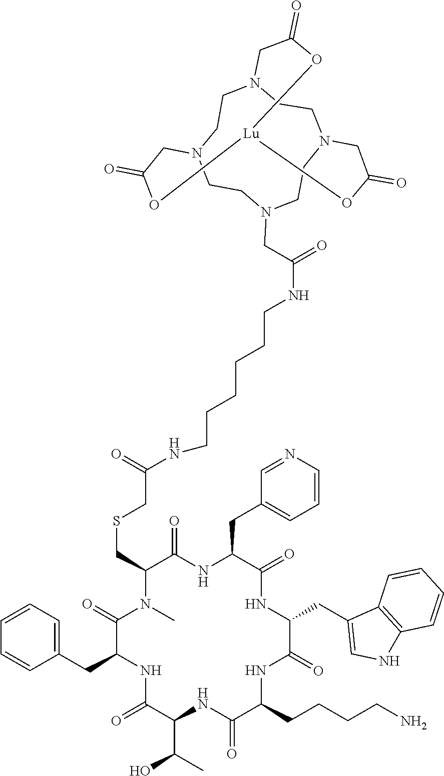 | 1541.6026 | ES-API- MS m/z: [M + H]+ = 1542.6 | Method 1; Purity: >90% |
| RB- 0002947 (152) | 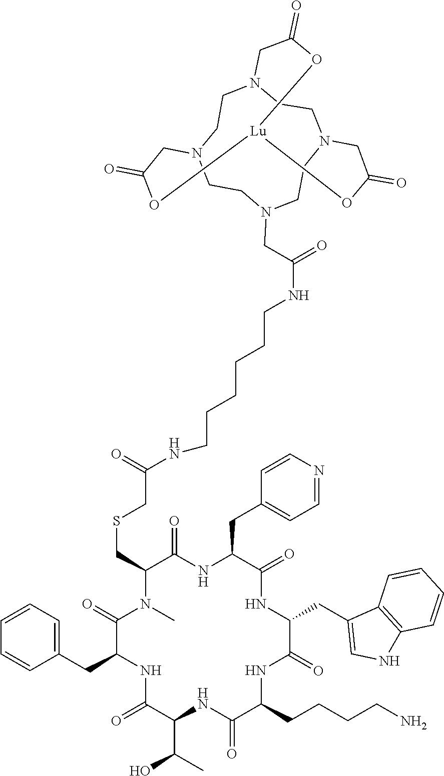 | 1541.6026 | ES-API- MS m/z: [M + H]+ = 1542.6 | Method 1; Purity: >90% |
| RB- 0003000 (153) | 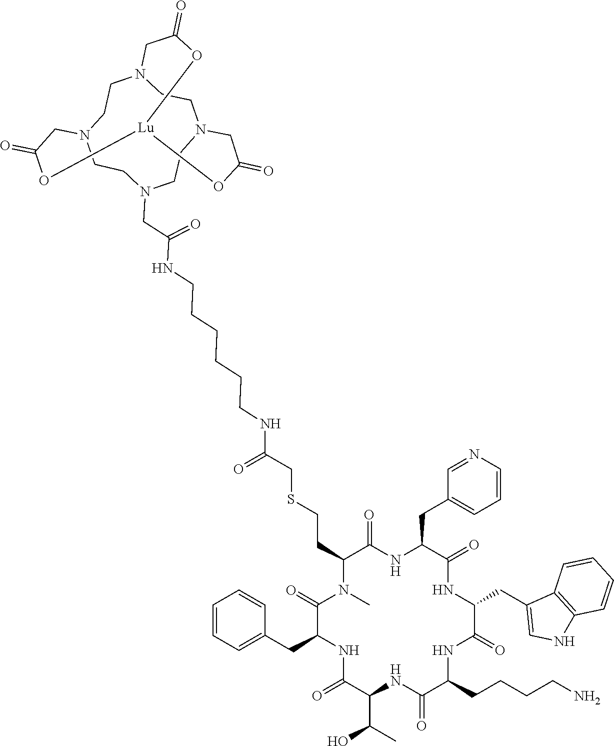 | 1555.6182 | ESI-MS m/z: [M + H]+ = 1556.8 | Method 1; Purity: 95.44% |
| RB- 0003002 (154) | 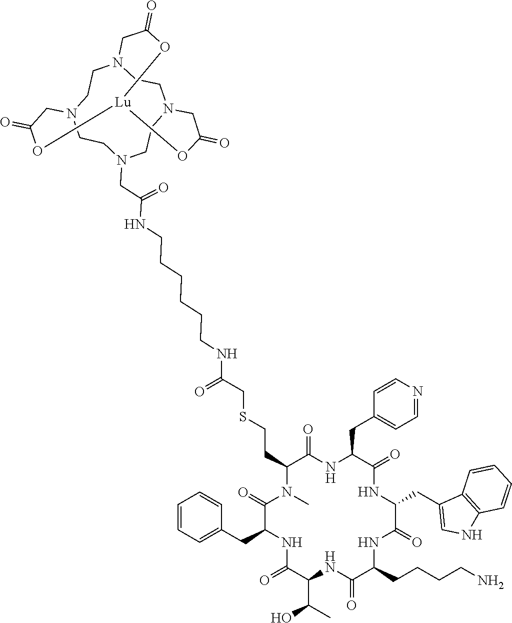 | 1555.6182 | ESI-MS m/z: [M + H]+ = 1556.8 | Method 1: Purity: 97.82% |
| RB- 0003136 (157) | 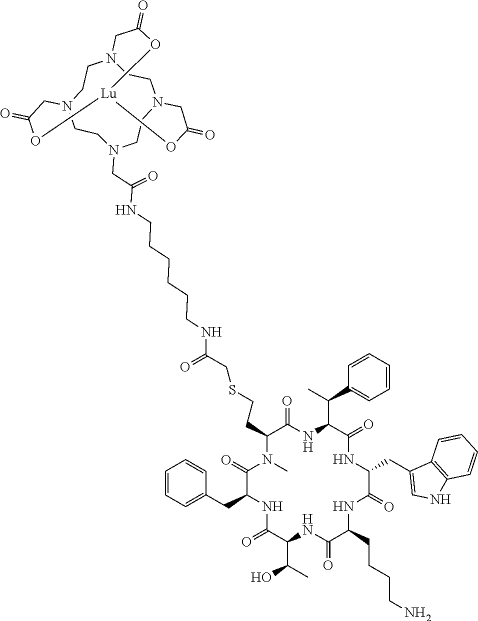 | 1568.6386 | ES-API- MS m/z: [M + H]+ = 1569.6 | Method 1; Purity: >90% |
| RB- 0003137 (158) | 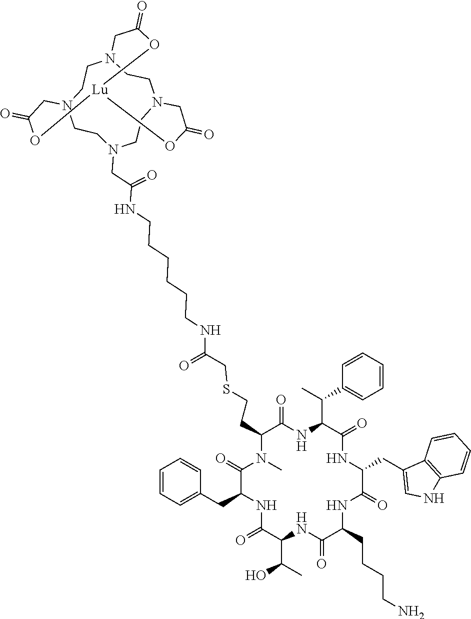 | 1568.6386 | ES-API- MS m/z: [M + H]+ = 1569.6 | Method 1; Purity: >90% |
| RB- 0003148 (159) | 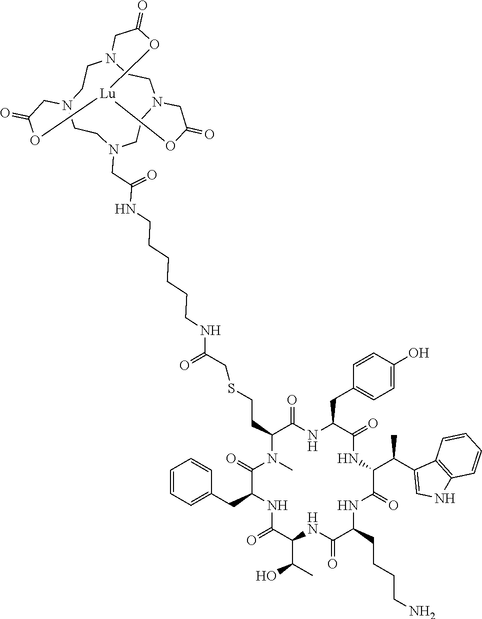 | 1584.6335 | ES-API- MS m/z: [M + H]+ = 1585.6 | Method 1; Purity: >90% |
| RB- 0003149 (160) | 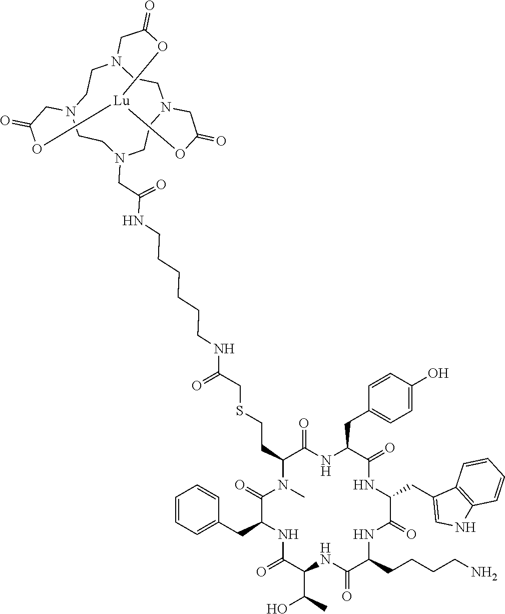 | 1570.6179 | ESI-MS m/z: [M + H]+ = 1571.6 | Method 1; Purity: 93.96% |
| RB- 0003164 (161) | 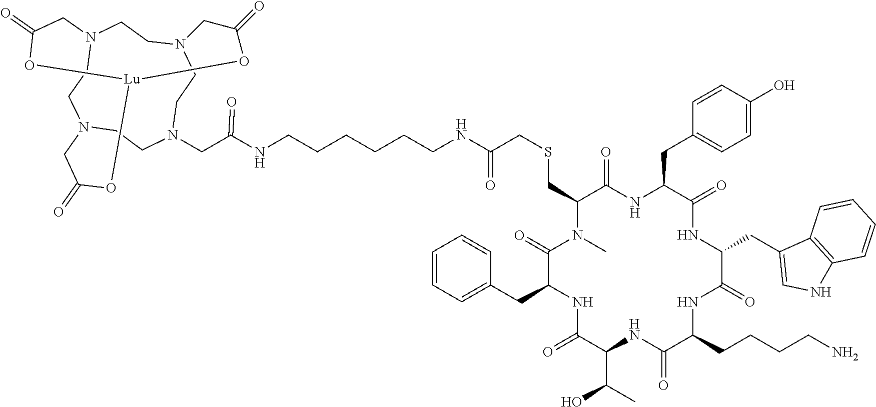 | 1556.6022 | ESI-MS m/z: [M + H]+ = 1557.6 | Method 1; Purity: 95.75% |
| RB- 0003276 (164) | 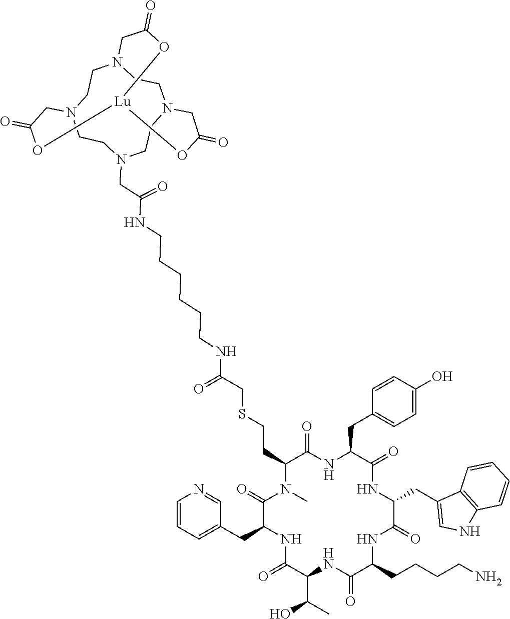 | 1571.6131 | ES-API- MS m/z: [M + H]+ = 1572.6 | Method 1; Purity: >90% |
| RB- 0003277 (165) | 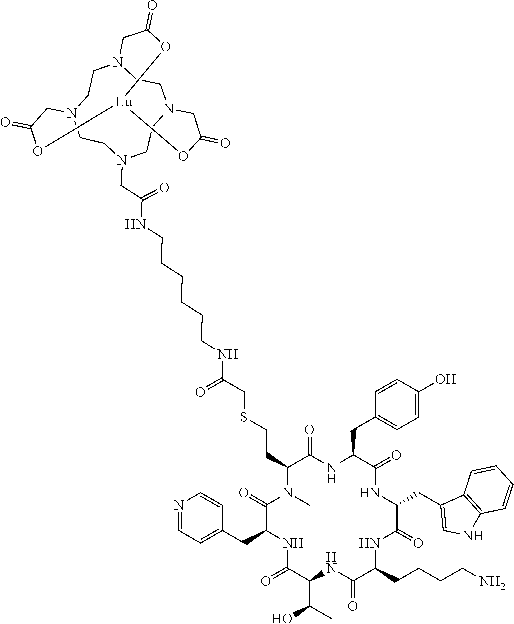 | 1571.6131 | ES-API- MS m/z: [M + H]+ = 1572.6 | Method 1; Purity: >90% |
| RB- 0003278 (166) | 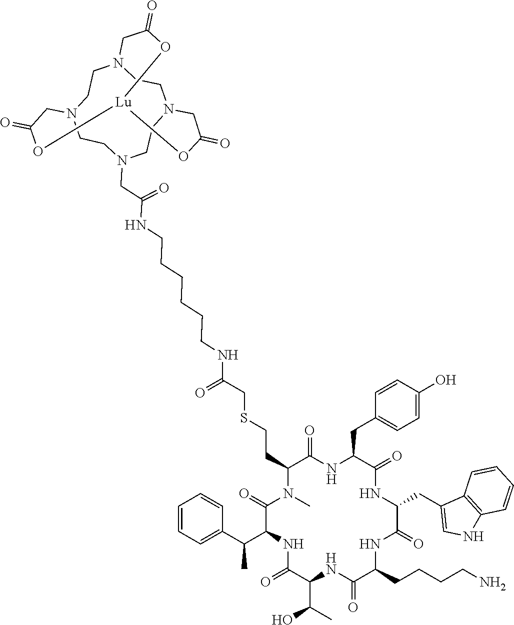 | 1584.6335 | ES-API- MS m/z: [M + H]+ = 1585.6 | Method 1; Purity: >90% |
| RB- 0003279 (167) | 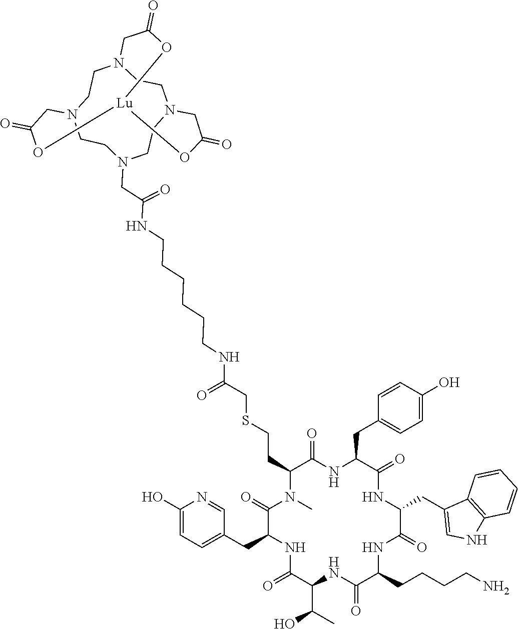 | 1587.6081 | ES-API- MS: m/z = 1588.6 | Method 1; Purity: >90% |
| RB- 0003280 (168) | 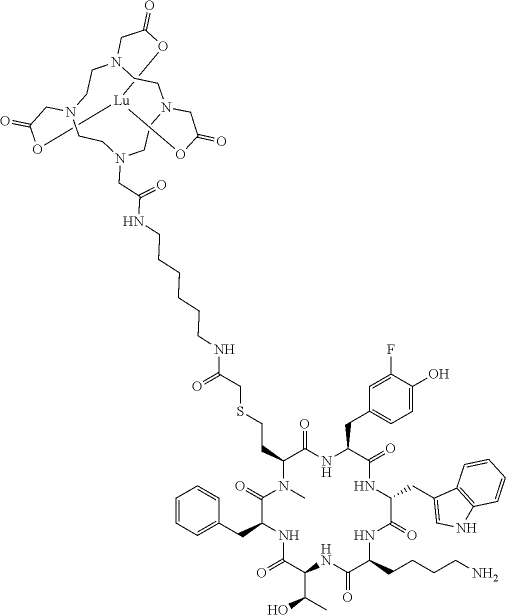 | 1588.6085 | ES-API- MS m/z: [M + H]+ = 1589.8 | Method 1; Purity: >90% |
| RB- 0003281 (169) | 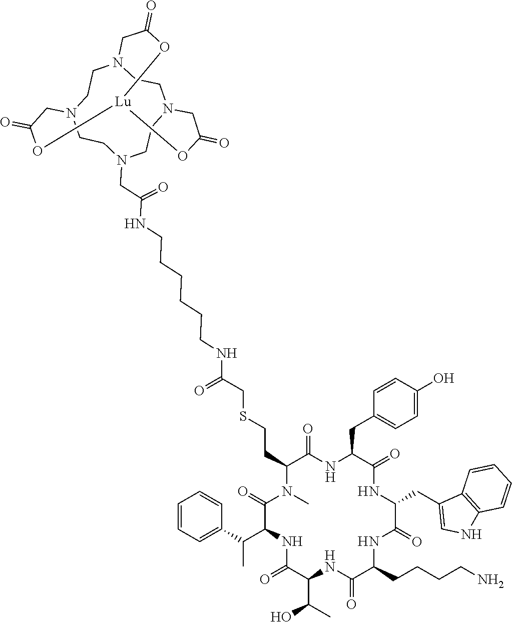 | 1570.6543 | ES-API- MS m/z: [M + H]+ = 1586.6 | Method 1; Purity: >90% |
| RB- 0003302 (174) | 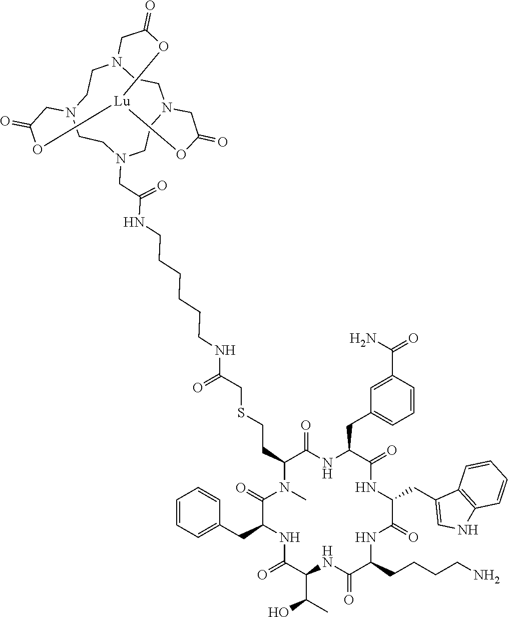 | 1597.6288 | ESI-MS m/z: [M/2 + H]+ = 800.0 | Method 1; Purity: 96.98% |
| RB- 0003304 (175) | 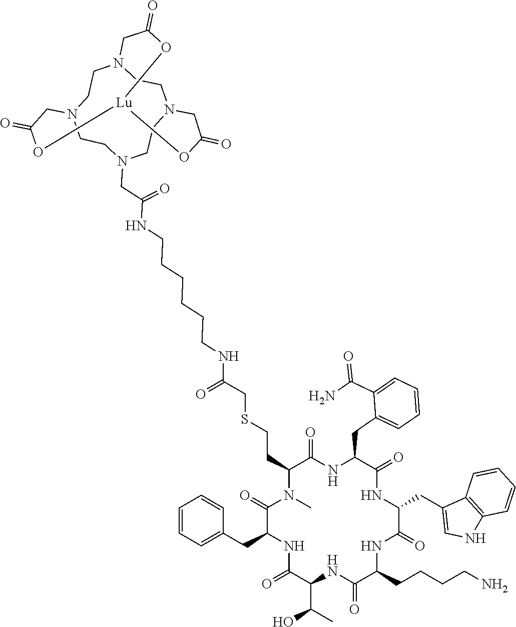 | 1597.6288 | ESI-MS m/z: [M/2 + H]+ = 800.0 | Method 1; Purity: 97.03% |
| RB- 0003306 (176) | 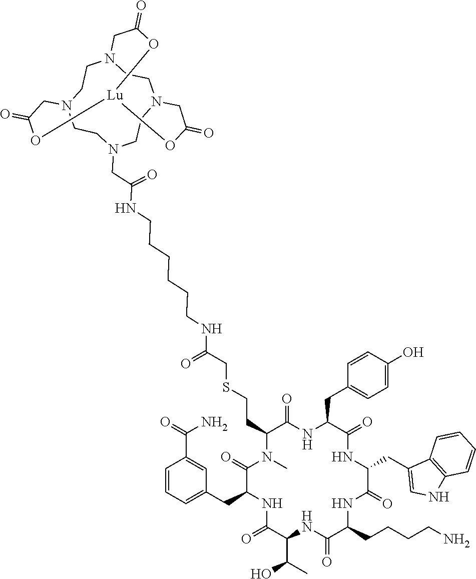 | 1613.6237 | ESI-MS m/z: [M/2 + H]+ = 808.4 | Method 1; Purity: 97.02% |
| RB- 0003309 (177) | 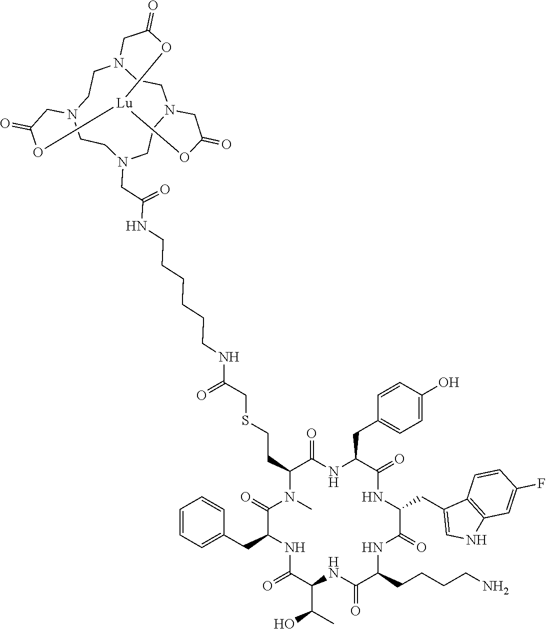 | 1588.6085 | ESI-MS m/z: [M/2 + H]+ = 795.5 | Method 1; Purity: 99.15% |
| RB- 0003357 (178) | 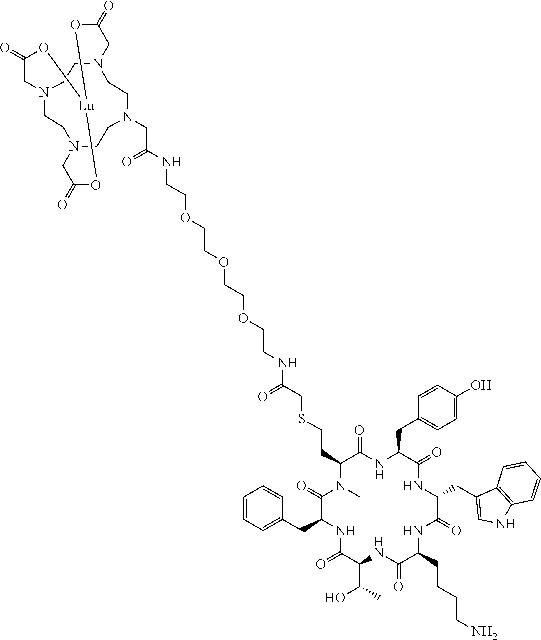 | 1646.6339 | ESI-MS m/z: [M + H]+ = 1647.6 | Method 1; Purity: 99.6% |
| RB- 0003374 (179) | 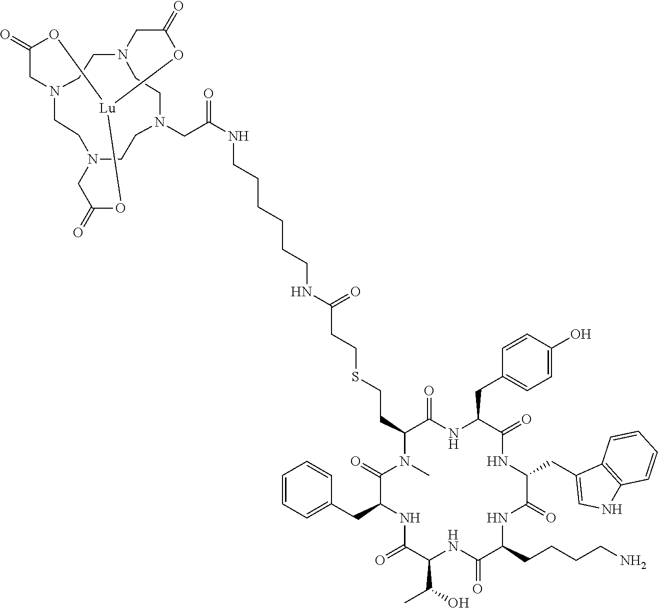 | 1584.6335 | ES-API- MS m/z: [M + H]+ = 1585.6 | Method 1; Purity: 91.56% |
| RB- 0003375 (180) | 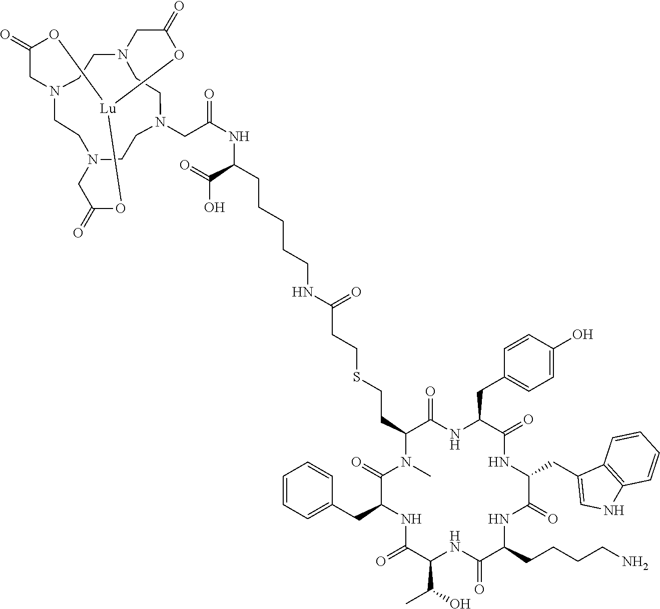 | 1628.6234 | ES-API- MS m/z: [M + H]+ = 1629.6 | Method 1: Purity: 97.68% |
| RB- 0003381 (181) | 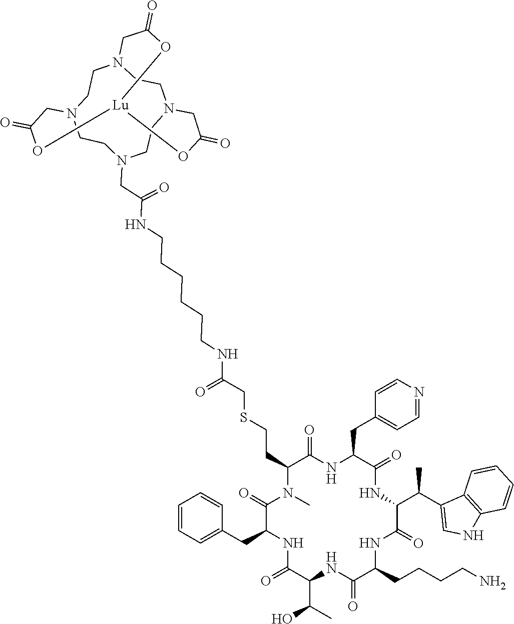 | 1569.6339 | ES-API- MS m/z: [M + H]+ = 1570.6 | Method 1; Purity: >90% |
| RB- 0003382 (182) | 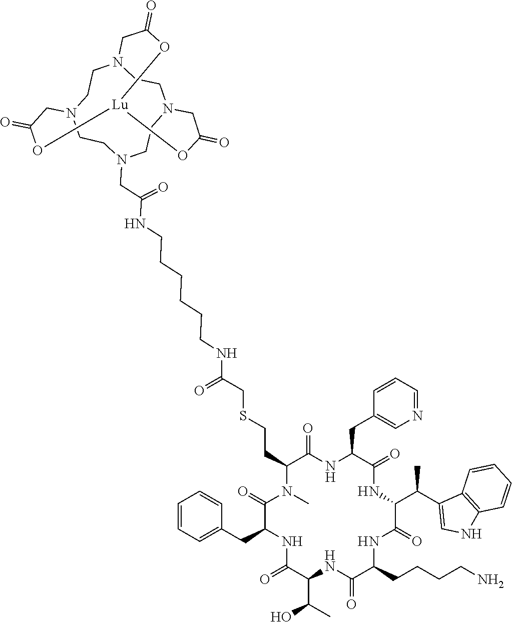 | 1569.6339 | ES-API- MS m/z: [M + H]+ = 1570.6 | Method 1; Purity: >90% |
| RB- 0003383 (183) | 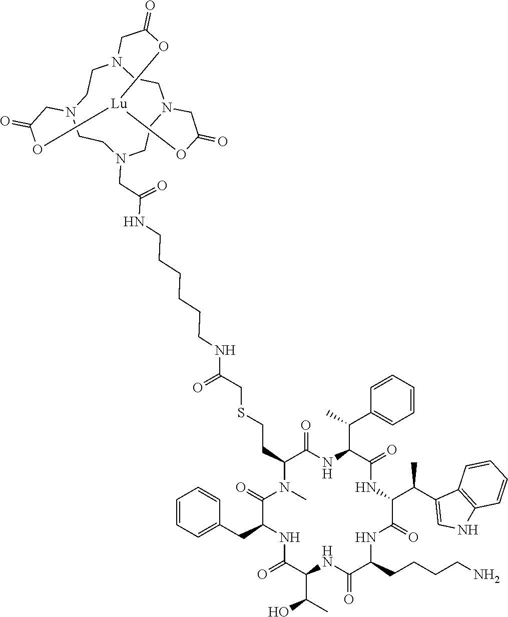 | 1582.6543 | ES-API- MS m/z: [M + H]+ = 1583.6 | Method 1; Purity: >90% |
| RB- 0003407 (184) | 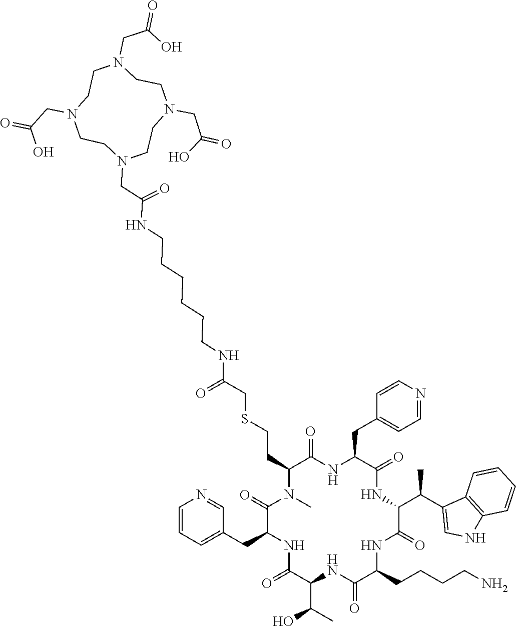 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H](Cc2cccnc2)NC1=O] | 1398.7118 | ES-API- MS m/z: [M + H]+ = 1399.7 | Method 1; Purity: >90% |
| RB- 0003408 (185) | 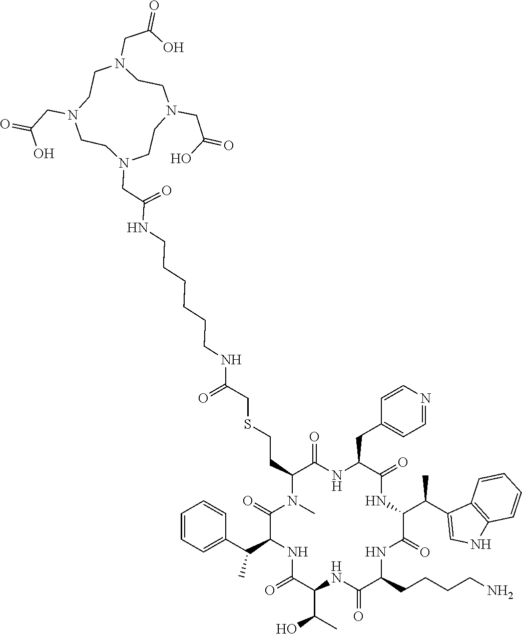 [SMILES: C[C@H](c1ccccc1)[C@@H]1NC(=O)[C@H]([C@@H](C)O)NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C1=O] | 1411.7322 | ES-API- MS m/z: [M + H]+ = 1412.8 | Method 1; Purity: >90% |
| RB- 0003409 (186) | 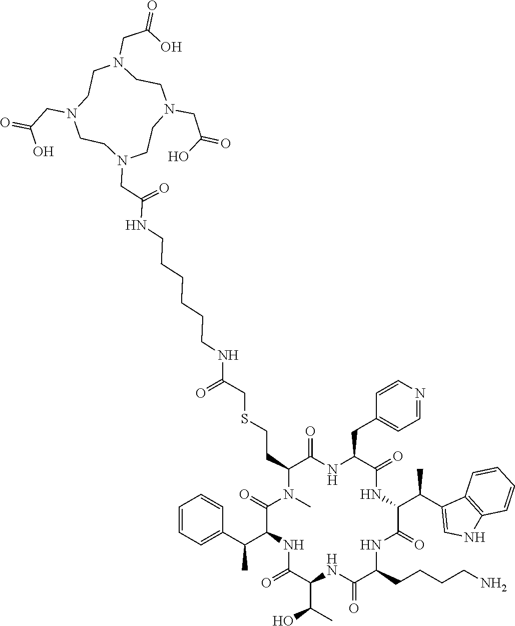 [SMILES: C[C@@H](O)[C@@H]1NC(=O)[C@H](CCCCN)NC(=O)[C@@H]([C@@H](C)c2c[nH]c3ccccc23)NC(=O)[C@H](Cc2ccncc2)NC(=O)[C@H](CCSCC(=O)NCCCCCCNC(=O)CN2CCN(CC(=O)O)CCN(CC(=O)O)CCN(CC(=O)O)CC2)N(C)C(=O)[C@H]([C@@H](C)c2ccccc2)NC1=O] | 1411.7322 | ES-API- MS m/z: [M + H]+ = 1412.8 | Method 1; Purity: >90% |
| RB- 0003443 (187) | 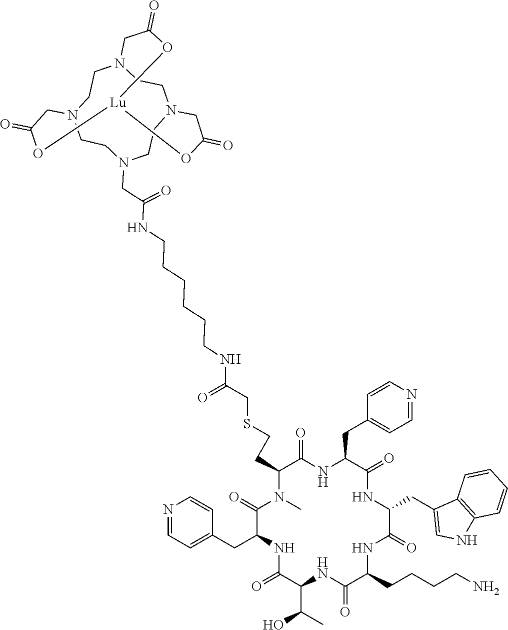 | 1556.6135 | ESI-MS m/z: [M + H]+ = 1557.9 | Method 1; Purity: 92% |
| RB- 0003445 (188) | 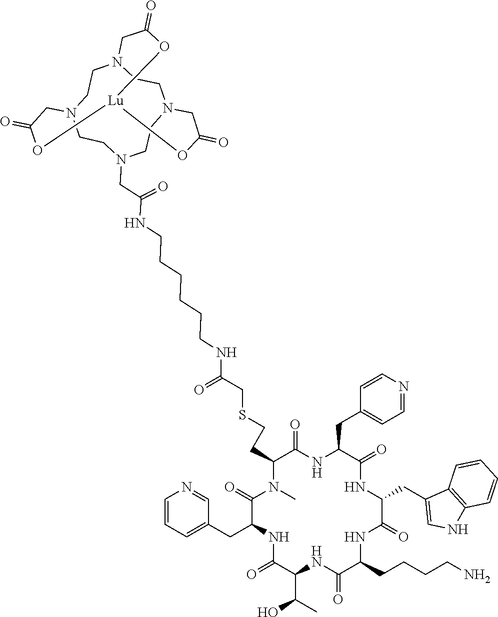 | 1556.6135 | ESI-MS m/z: [M + H]+ = 1557.9 | Method 1; Purity: 98% |
| RB- 0003447 (189) | 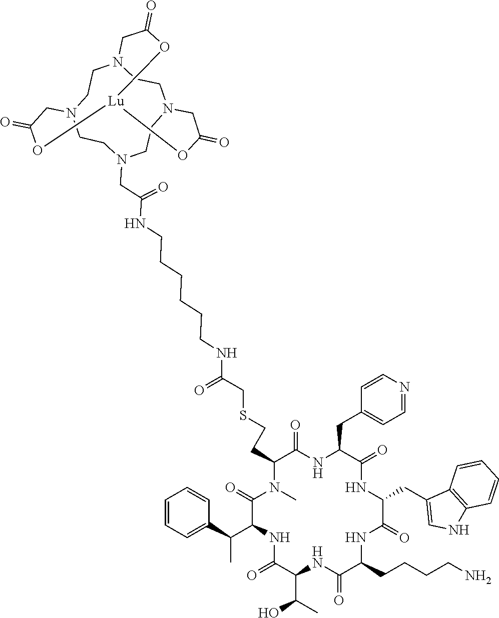 | 1569.6339 | ESI-MS m/z: [M + H]+ = 1570.9 | Method 1; Purity: 96% |
| RB- 0003449 (190) | 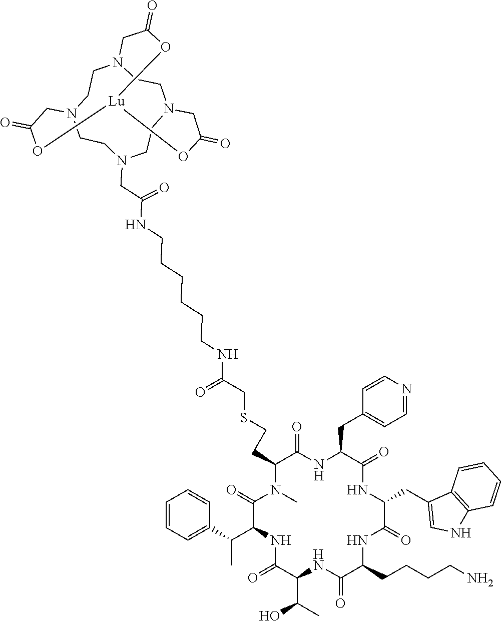 | 1569.6339 | ESI-MS m/z: [M + H]+ = 1570.9 | Method 1: Purity: 97% |
| RB- 0003451 (191) | 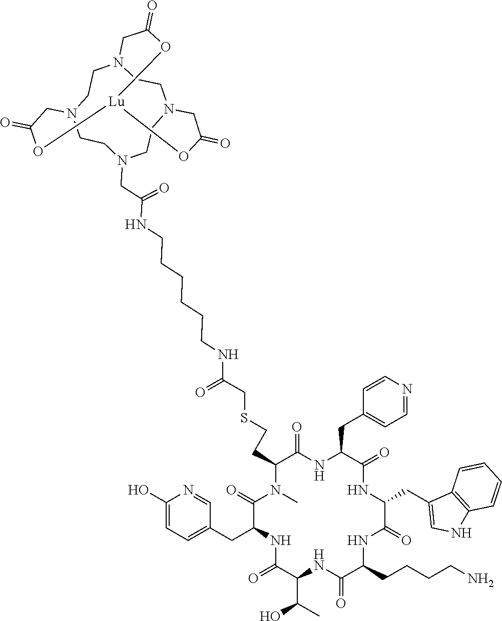 | 1572.6084 | ESI-MS m/z: [M/2 + H]+ = 787.6 | Method 1; Purity: 96% |
| RB- 0003453 (192) | 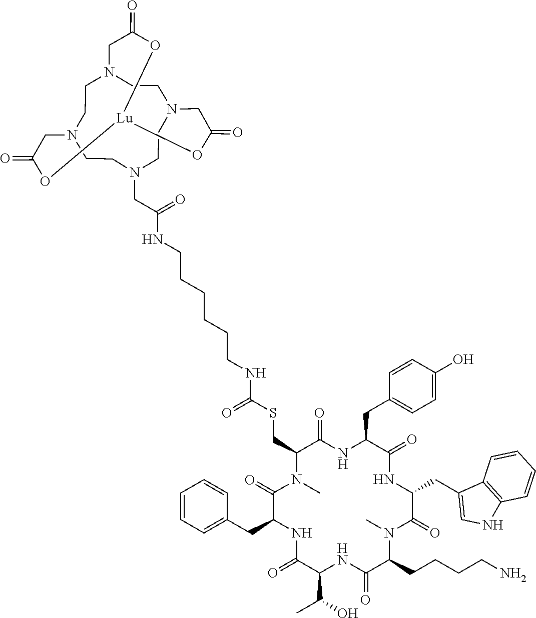 | 1584.6335 | ESI-MS m/z: [M + H]+ = 1585.9 | Method 1; Purity: 98% |
| RB- 0003455 (193) | 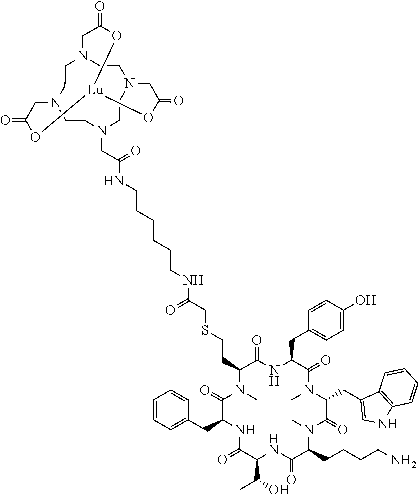 | 1598.6492 | ESI-MS m/z: [M/2 + H]+ = 800.6 | Method 1: Purity: 98% |
| RB- 0003464 (194) | 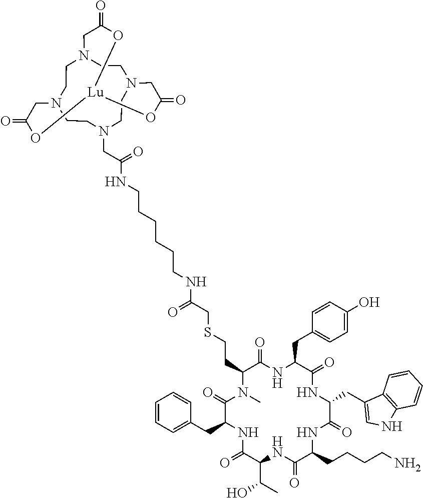 | 1570.6179 | ES-API- MS m/z: [M + H]+ = 1571.6 | Method 1; Purity: 99% |
| RB- 0003482 (195) | 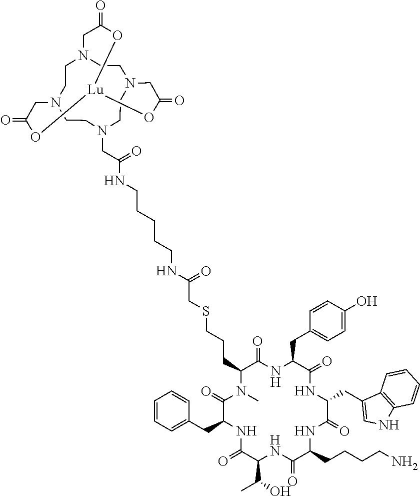 | 1570.6179 | ESI-MS m/z: [M + H]+ = 1571.9 | Method 1; Purity: 97.22% |
| RB- 0003484 (196) | 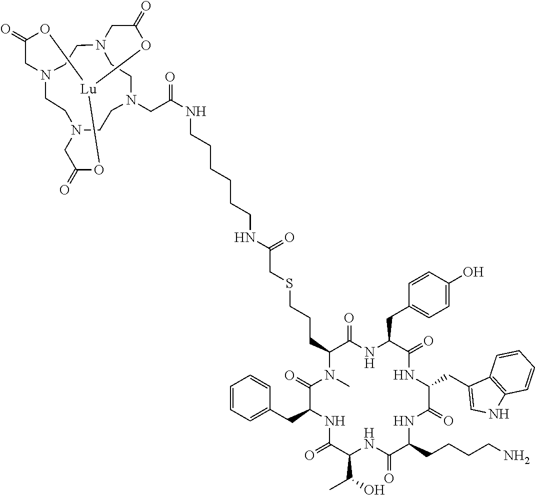 | 1584.6335 | ESI-MS m/z: [M + H]+ = 1585.9 | Method 1: Purity: 94.36% |
| RB- 0003486 (197) | 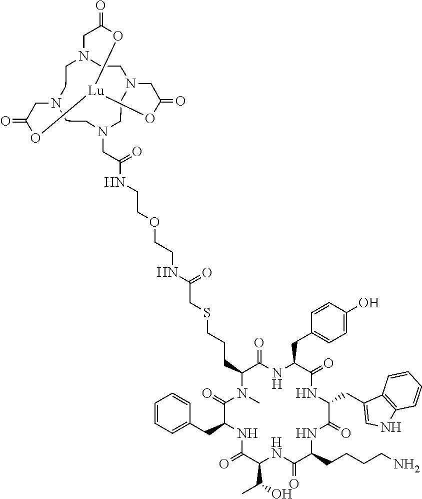 | 1572.5972 | ESI-MS m/z: [M + H]+ = 1573.8 | Method 1: Purity: 95.36% |
| RB- 0003504 (198) | 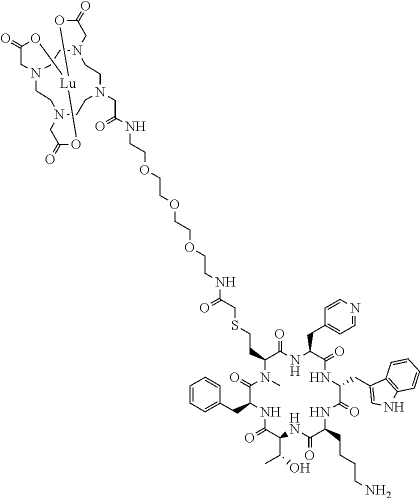 | 1631.6343 | ES-API- MS m/z: [M + H]+ = 1632.6 | Method 1; Purity: 93% |
| RB- 0004538 (200) | 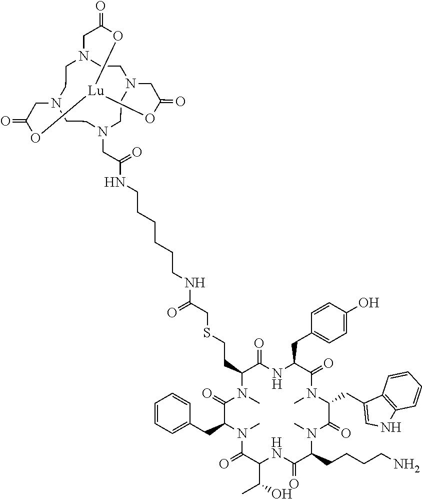 | 1612.6648 | ESI-MS m/z: [M + H]+ = 1613.7 | Method 1; Purity: 97.71% |
| RB- 0004540 (201) | 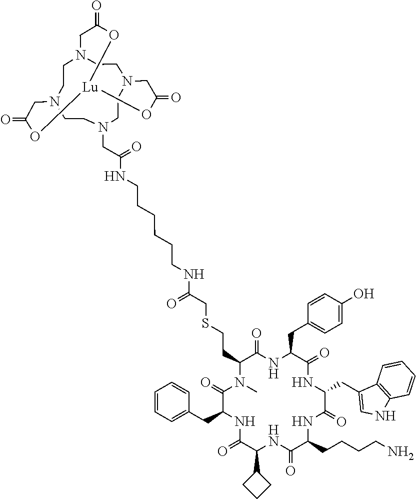 | 1580.6386 | ESI-MS m/z: [M + H]+ = 1581.9 | Method 1; Purity: 96.93% |
| RB- 0004609 (202) | 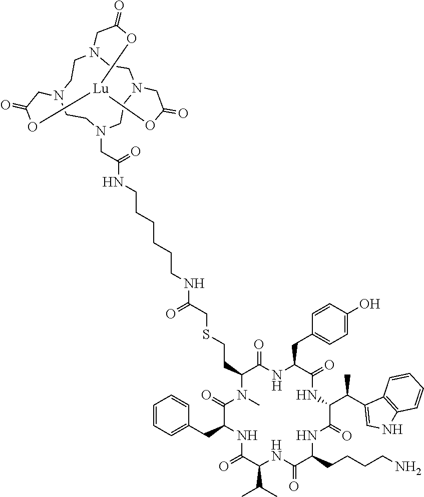 | 1582.6543 | ESI-MS m/z: [M + H]+ = 1583.7 | Method 1; Purity: 96.04% |
| RB- 0004611 (203) | 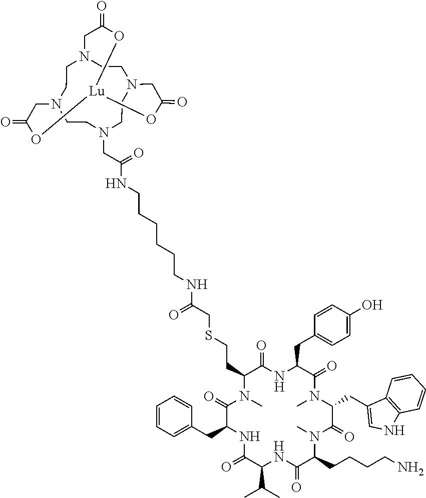 | 1596.6699 | ESI-MS m/z: [M + H]+ = 1597.7 | Method 1; Purity: 99.01% |
| RB- 0004613 (204) | 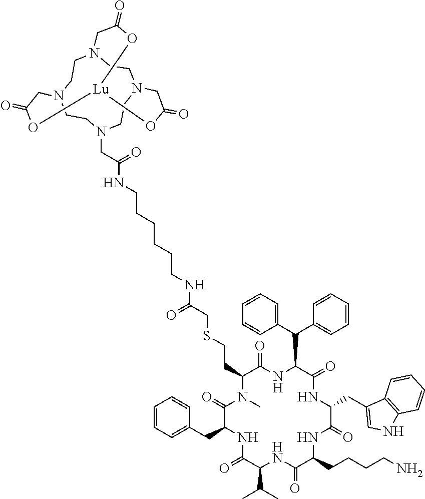 | 1628.675 | ESI-MS m/z: [M + H]+ = 1629.9 | Method 1; Purity: 98.42% |
| RB- 0004615 (205) | 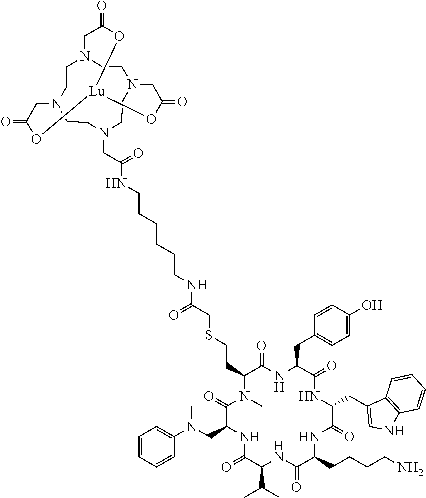 | 1597.6652 | ESI-MS m/z: [M + H]+ = 1598.9 | Method 1; Purity: 97.09% |
| RB- 0004617 (206) | 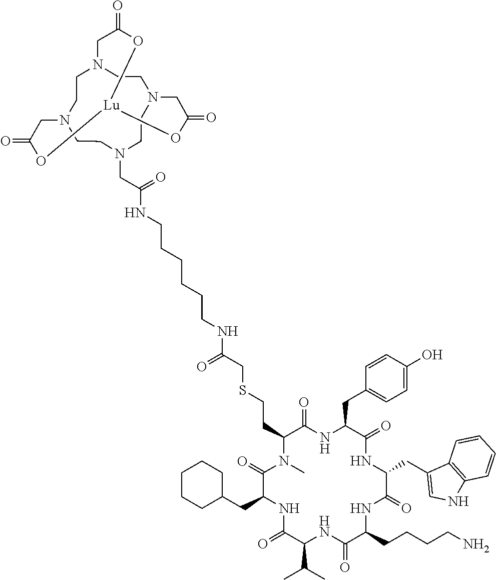 | 1574.6856 | ESI-MS m/z: [M + H]+ = 1575.9 | Method 1; Purity: 96.85% |
| RB- 0004619 (207) | 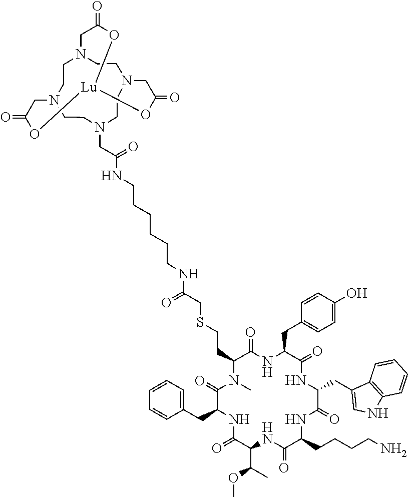 | 1584.6335 | ESI-MS m/z: [M + H]+ = 1585.9 | Method 1: Purity: 96.26% |
| RB- 0004621 (208) | 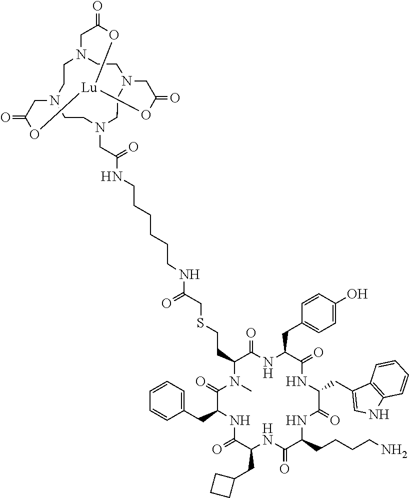 | 1594.6543 | ESI-MS m/z: [M + H]+ = 1595.9 | Method 1; Purity: 97.34% |
| RB- 0004623 (209) | 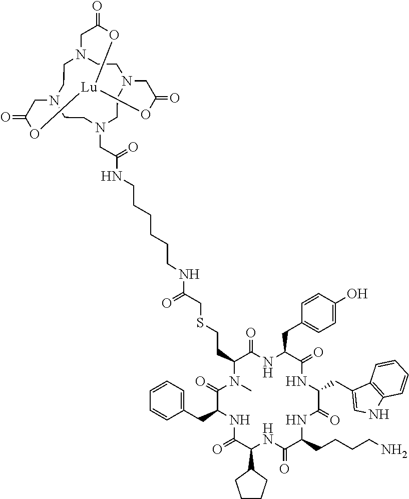 | 1594.6543 | ESI-MS m/z: [M + H]+ = 1595.9 | Method 1; Purity: 97% |
| RB- 0004819 (210) | 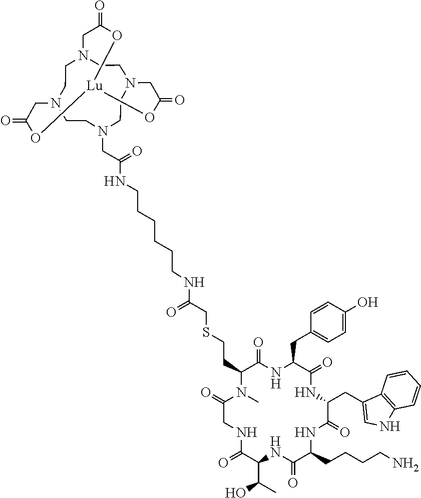 | 1480.5709 | ESI-MS m/z: [M + H]+ = 1481.6 | Method 1; Purity: 97.08% |
| RB- 0004821 (211) | 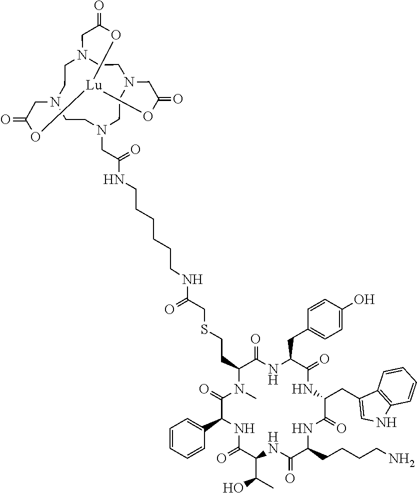 | 1556.6022 | ESI-MS m/z: [M + H]+ = 1557.6 | Method 1; Purity: 95.78% |
| RB- 0004823 (212) | 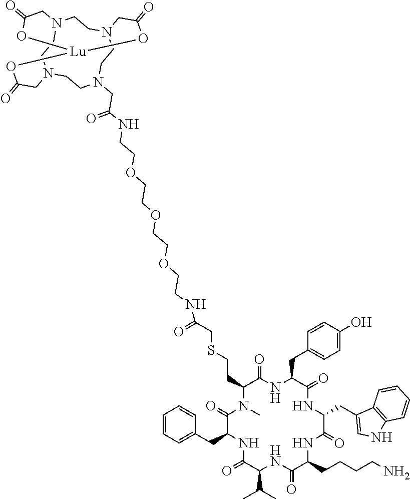 | 1644.6547 | ESI-MS m/z: [M + H]+ = 1645.7 | Method 1; Purity: 97.36% |
| RB- 0004825 (213) | 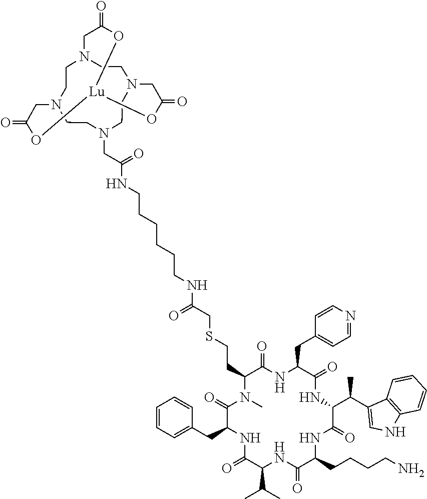 | 1567.6546 | ESI-MS m/z: [M + H]+ = 1568.7 | Method 1; Purity: 95.33% |
| RB- 0004864 (214) | 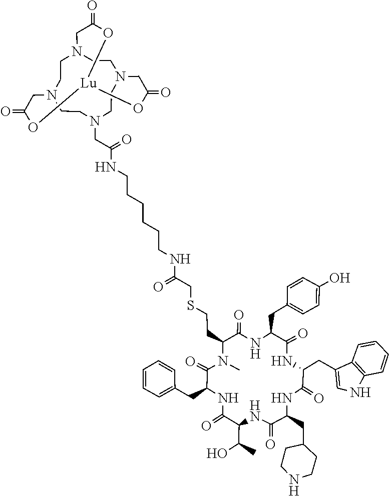 | 1596.6335 | ESI-MS m/z: [M + H]+ = 1597.7 | Method 1; Purity: 96.92% |
| RB- 0004866 (215) | 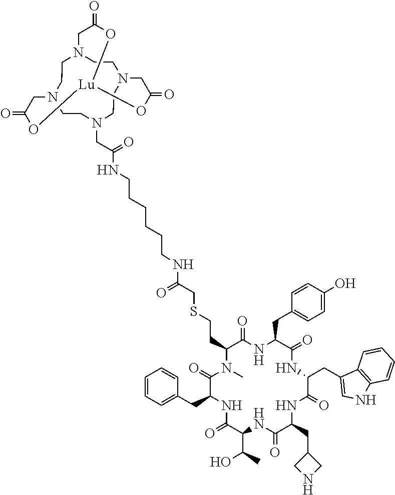 | 1568.6022 | ESI-MS m/z: [M + H]+ = 1569.6 | Method 1; Purity: 95.22% |
| RB- 0004868 (216) | 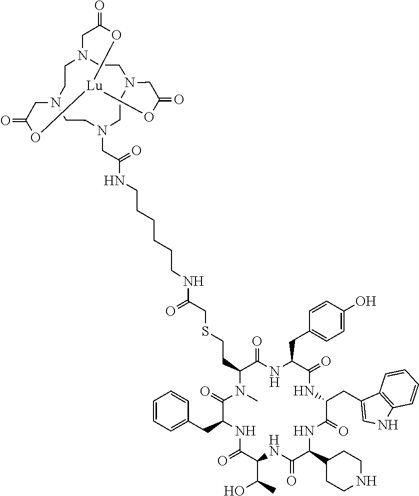 | 1582.6179 | ESI-MS m/z: [M + H]+ = 1583.6 | Method 1; Purity: 96.28% |
| RB- 0005247 (217) | 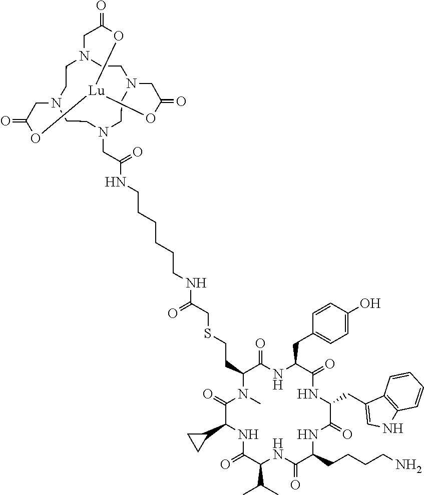 | 1518.623 | ESI-MS m/z: [M + H]+ = 1519.6 | Method 1; Purity: 97.2% |
| RB- 0005253 (218) | 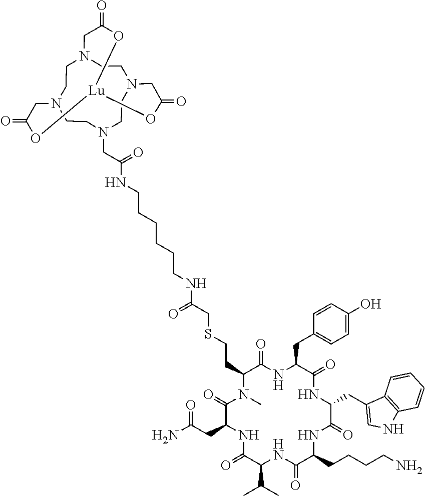 | 1535.6131 | ESI-MS m/z: [M + H]+ = 1536.6 | Method 1; Purity: 96.11% |
| RB- 0005255 (219) | 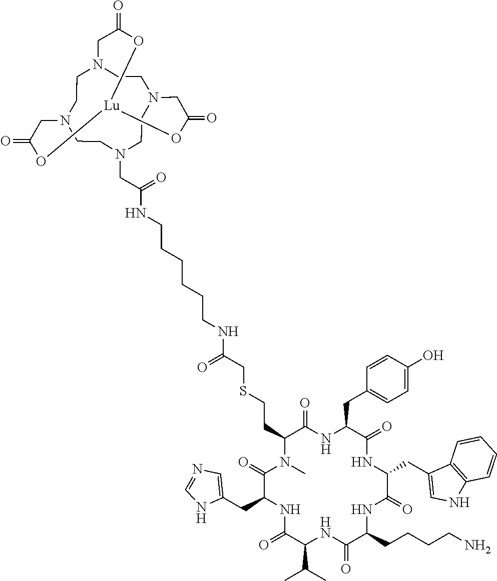 | 1558.6291 | ESI-MS m/z: [M + H]+ = 1559.6 | Method 1; Purity: 98.51% |
| RB- 0005266 (220) | 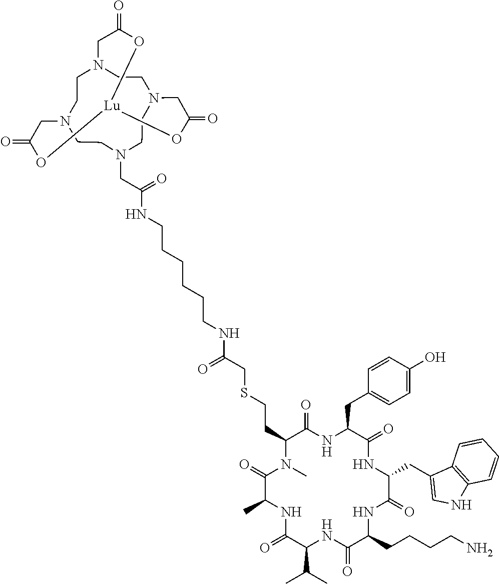 | 1492.6073 | ESI-MS m/z: [M + H]+ = 1493.6 | Method 1; Purity: 96.54% |
| RB- 0005270 (221) | 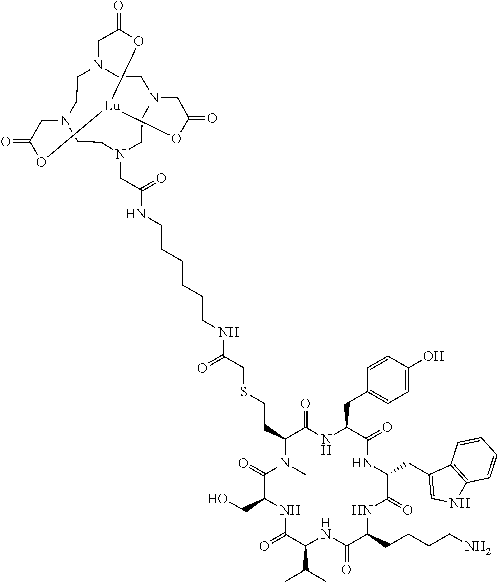 | 1508.6022 | ESI-MS m/z: [M + H]+ = 1509.6 | Method 1; Purity: 95.11% |
| RB- 0005272 (222) | 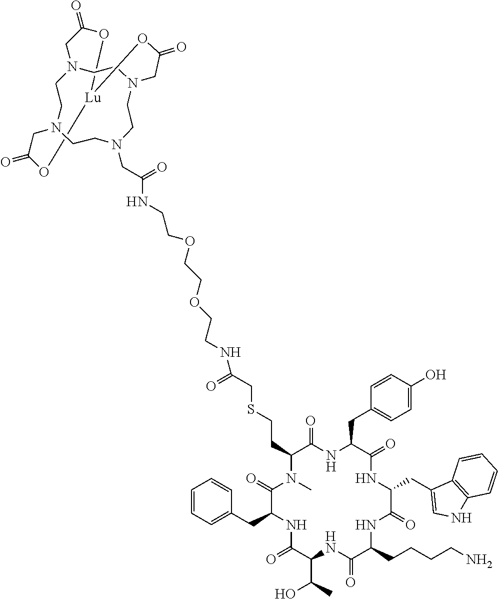 | 1602.6077 | ESI-MS m/z: [M + H]+ = 1603.6 | Method 1: Purity: 97.78% |
| RB- 0005274 (223) | 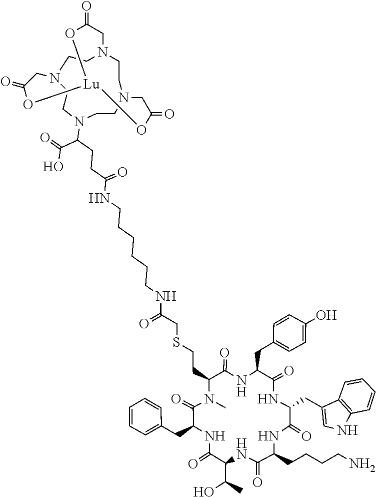 | 1642.639 | ESI-MS m/z: [M + H]+ = 1643.6 | Method 1: Purity: 98.71% |
| RB- 0005276 (224) | 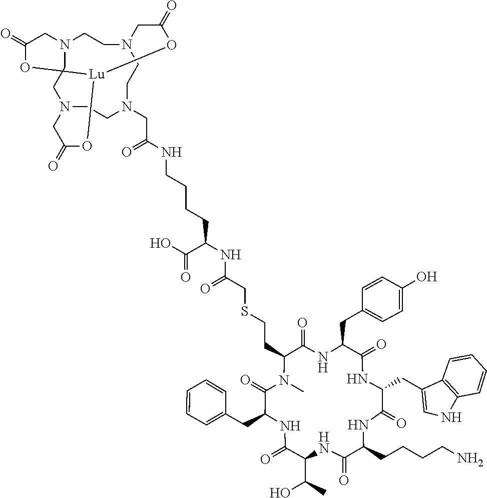 | 1600.5921 | ESI-MS m/z: [M + H]+ = 1601.6 | Method 1; Purity: 97.15% |
| RB- 0005278 (225) | 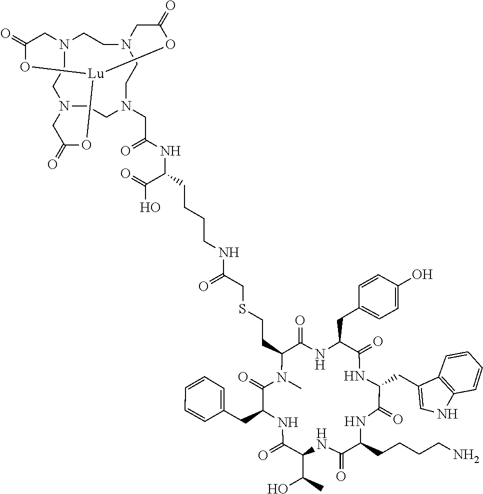 | 1600.5921 | ESI-MS m/z: [M + H]+ = 1601.6 | Method 1: Purity: 98.04% |
| RB- 0005284 (226) | 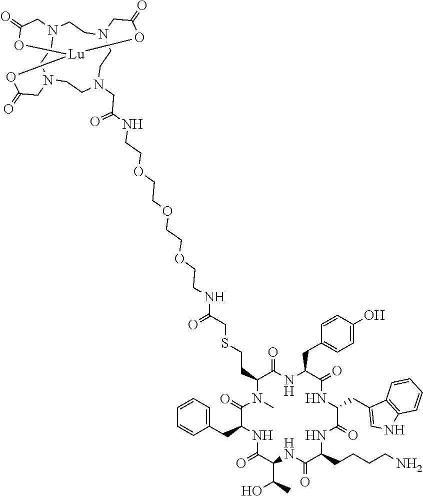 | 1646.6339 | ESI-MS m/z: [M + H]+ = 1647.6 | Method 1; Purity: 98.32% |
| RB- 0005286 (227) | 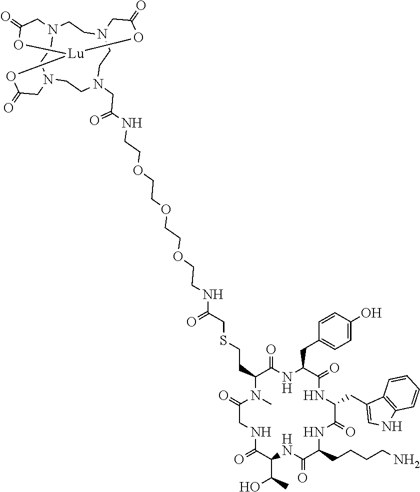 | 1556.587 | ESI-MS m/z: [M + H]+ = 1557.6 | Method 1; Purity: 98.02% |
| RB- 0005330 (228) | 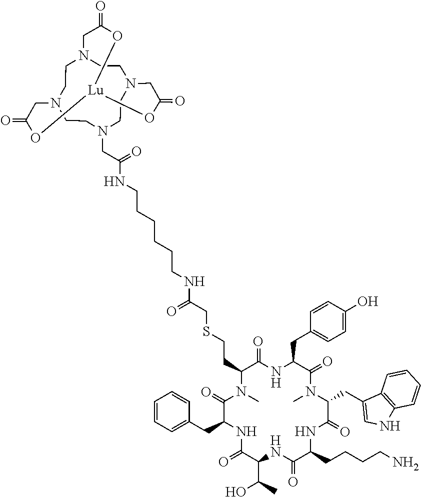 | 1584.6335 | ESI-MS m/z: [M + H]+ = 1585.6 | Method 1; Purity: 96.11% |
| RB- 0005502 (229) | 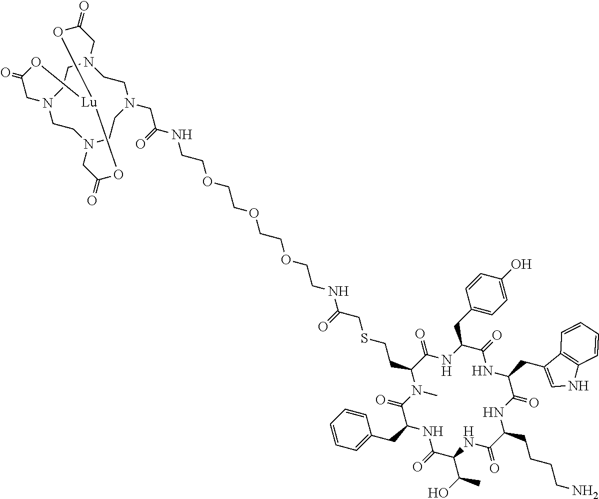 | 1646.6339 | ESI-MS m/z: [M + H]+ = 1647.6 | Method 1; Purity: 97.89% |
| RB- 0005692 (230) | 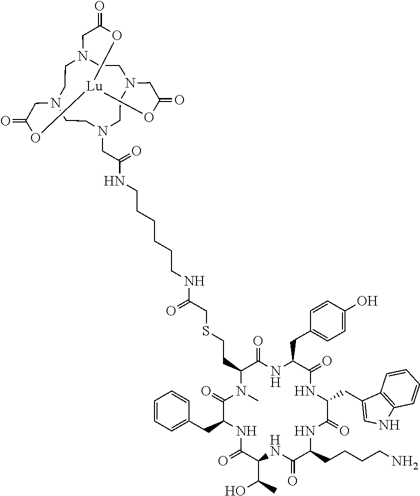 | 1584.6335 | ESI-MS m/z: [M + H]+ = 1585.6 | Method 1; Purity: 97.43% |
| RB- 0005694 (231) | 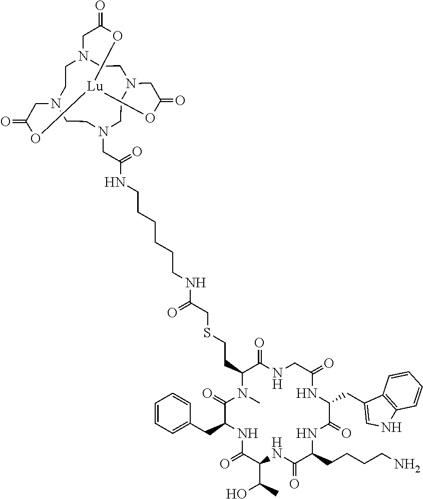 | 1464.576 | ESI-MS m/z: [M + H]+ = 1465.6 | Method 1; Purity: 96.78% |
| RB- 0005696 (232) | 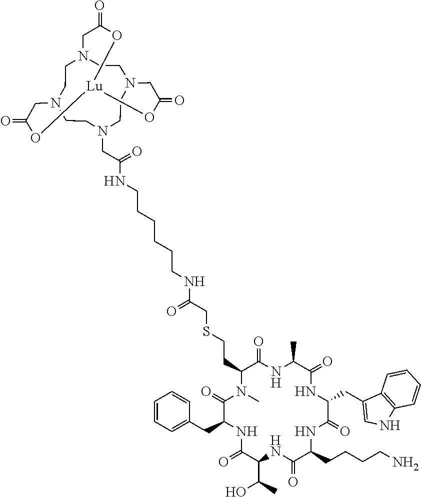 | 1478.5917 | ESI-MS m/z: [M + H]+ = 1479.6 | Method 1; Purity: 96.58% |
| RB- 0005738 (239) | 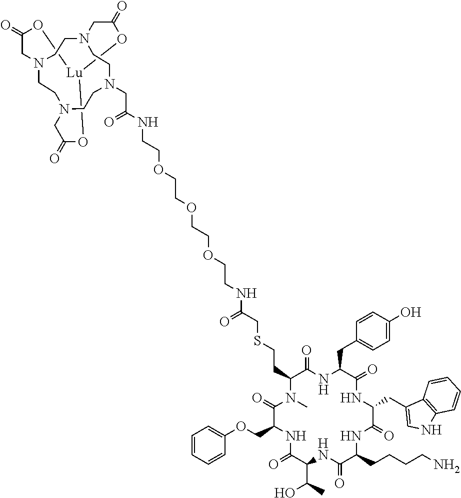 | 1662.6288 | ESI-MS m/z: [M + H]+ = 1663.6 | Method 1; Purity: 97.79% |
| RB- 0005740 (240) | 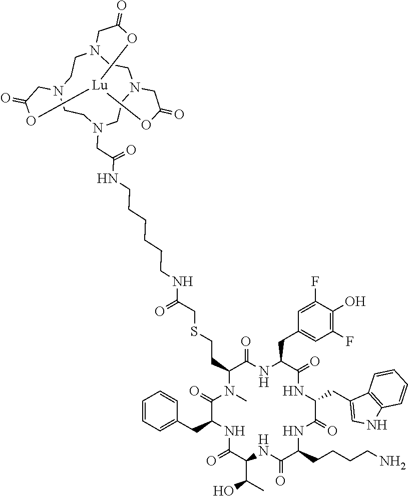 | 1606.599 | ESI-MS m/z: [M + H]+ = 1607.6 | Method 1; Purity: 96.12% |
| RB- 0005744 (241) | 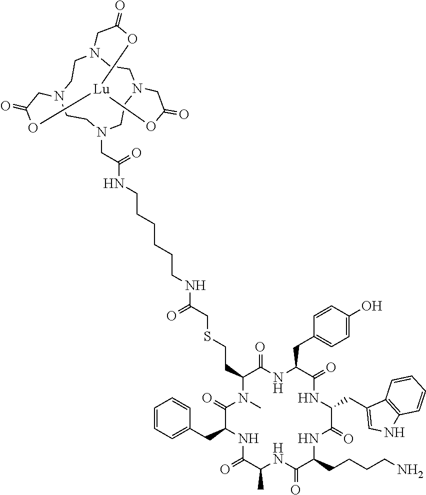 | 1540.6073 | ESI-MS m/z: [M + H]+ = 1541.6 | Method 1; Purity: 97.52% |
| RB- 0005760 (242) | 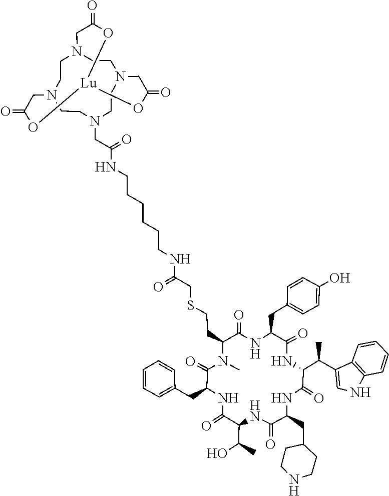 | 1610.6492 | ESI-MS m/z: [M + H]+ = 1611.6 | Method 1; Purity: 98% |
| RB- 0005761 (243) | 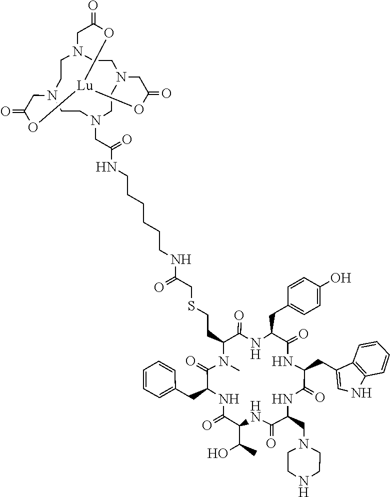 | 1596.6335 | ES-API- MS m/z: [M + H]+ = 1597.6 | Method 1; Purity: 98% |
| RB- 0005799 (245) | 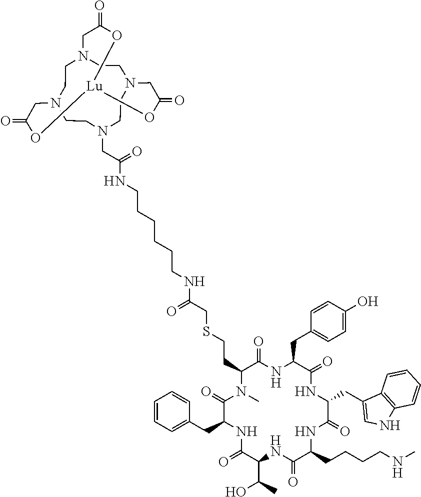 | 1584.6335 | ESI-MS m/z: [M + H]+ = 1585.6 | Method 1: Purity: 96.35% |
| RB- 0005801 (246) | 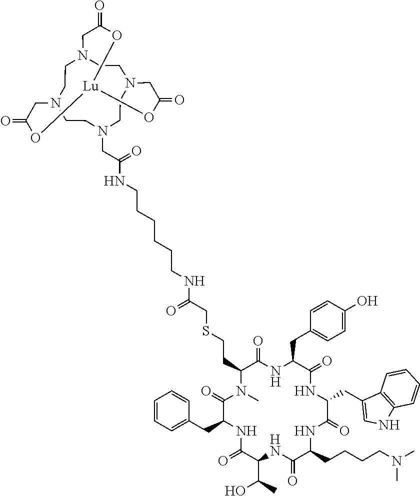 | 1598.6492 | ESI-MS m/z: [M + H]+ = 1599.7 | Method 1; Purity: 96.06% |
| RB- 0005805 (247) | 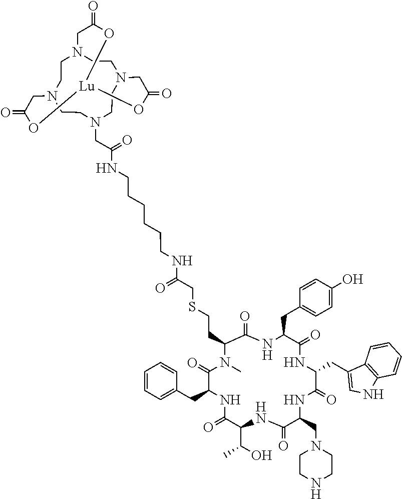 | 1597.6288 | ESI-MS m/z: [M + H]+ = 1598.6 | Method 1; Purity: 97.05% |
| RB- 0005807 (248) | 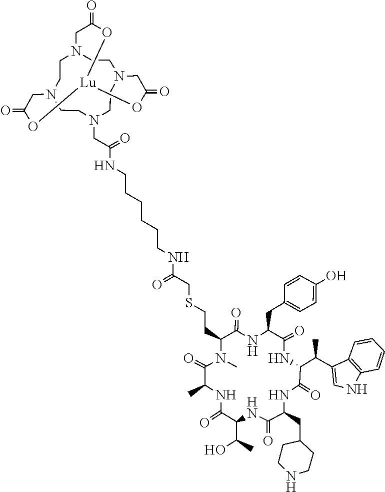 | 1534.6179 | ESI-MS m/z: [M + H]+ = 1535.6 | Method 1: Purity: 97.04% |
| RB- 0005809 (249) | 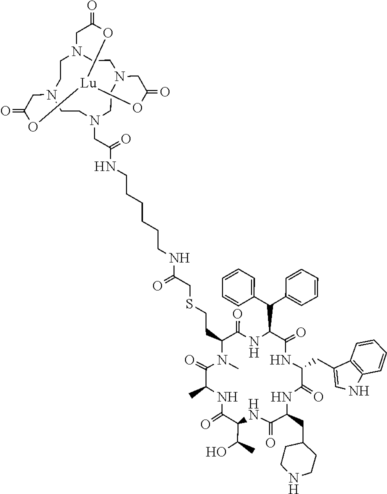 | 1580.6386 | ESI-MS m/z: [M + H]+ = 1581.7 | Method 1; Purity: 97.32% |
| RB- 0005827 (250) | 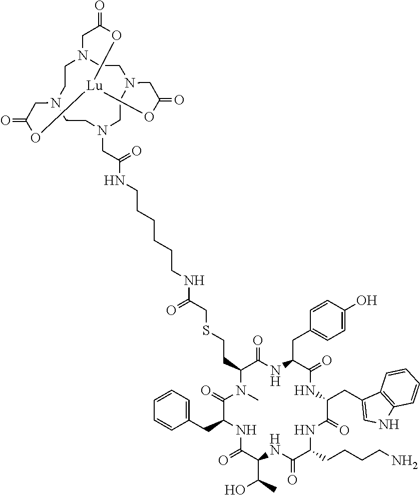 | 1570.6179 | ES-API- MS m/z: [M + H]+ = 1571.6 | Method 1: Purity: 95% |
| RB- 0005858 (253) | 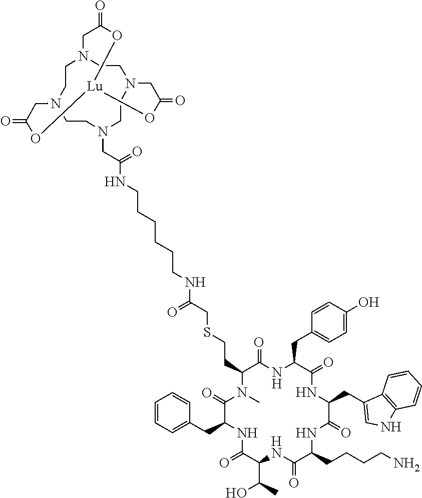 | 1571.6131 | ESI-MS m/z: [M + H]+ = 1572.6 | Method 1; Purity: 98.61% |
| RB- 0005864 (254) | 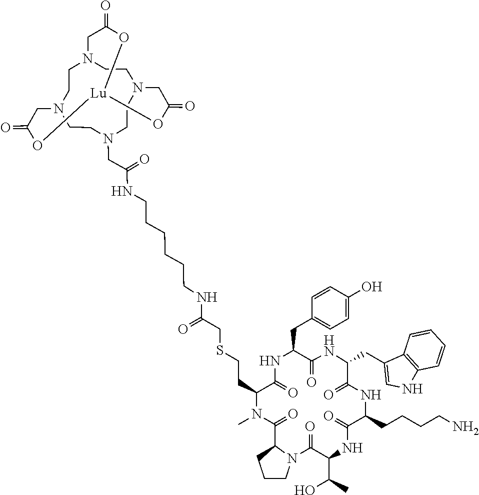 | 1520.6022 | ESI-MS m/z: [M + H]+ = 1521.6 | Method 1; Purity: 96.74% |
| RB- 0005866 (255) | 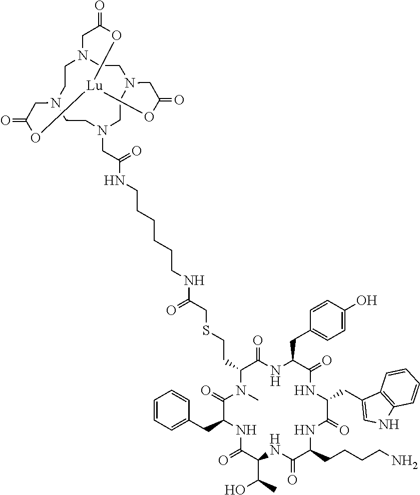 | 1570.6179 | ESI-MS m/z: [M + H]+ = 1571.6 | Method 1; Purity: 97.01% |
| RB- 0005897 (257) | 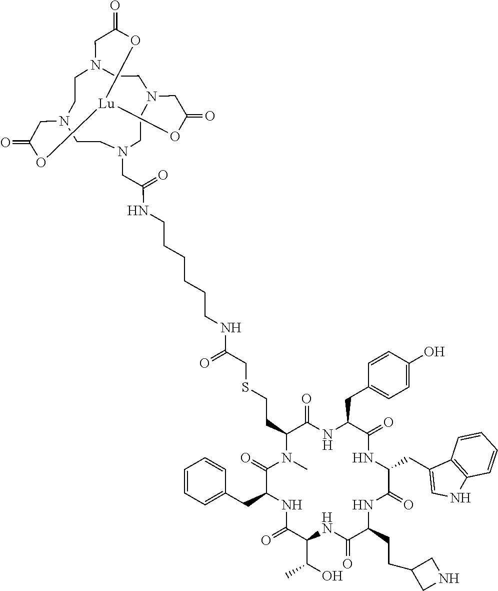 | 1582.6179 | ESI-MS m/z: [M + H]+ = 1583.6 | Method 1: Purity: 95.57% |
| RB- 0005899 (258) | 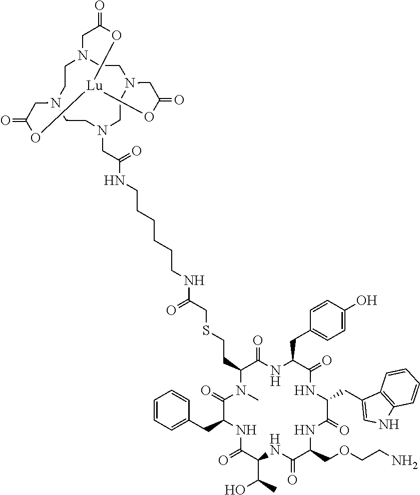 | 1572.5972 | ESI-MS m/z: [M + H]+ = 1573.6 | Method 1; Purity: 95.09% |
| RB- 0005903 (259) | 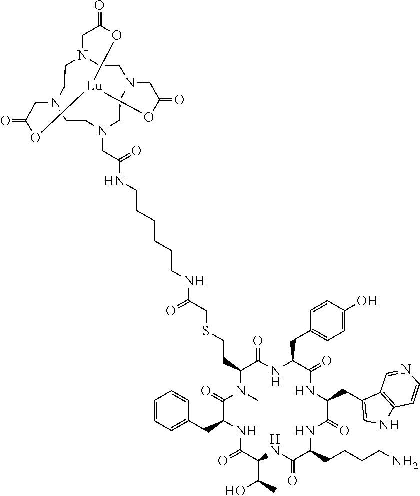 | 1571.6131 | ESI-MS m/z: [M + H]+ = 1572.6 | Method 1; Purity: 98.15% |
| RB- 0005905 (260) | 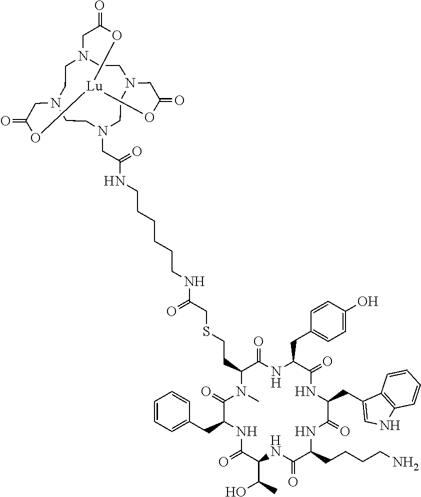 | 1571.6131 | ESI-MS m/z: [M + H]+ = 1572.6 | Method 1: Purity: 95.25% |
| RB- 0005907 (261) | 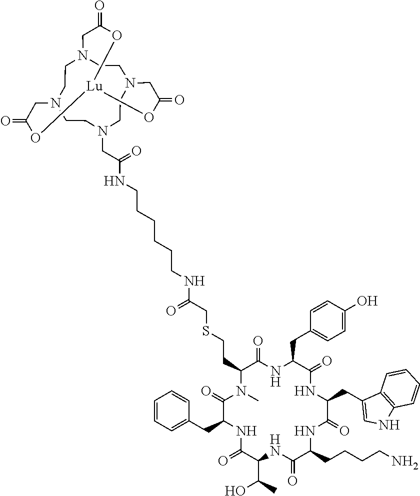 | 1571.6131 | ESI-MS m/z: [M + H]+ = 1572.6 | Method 1: Purity: 98.15% |
| RB- 0005990 (265) | 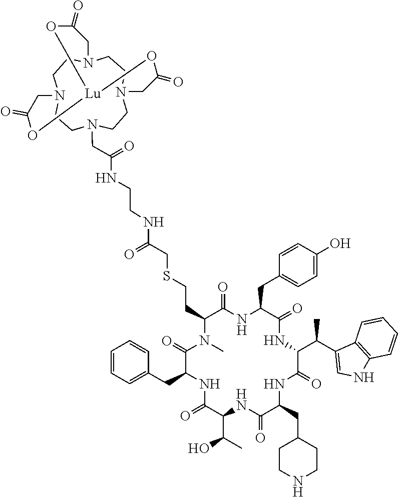 | 1554.5866 | ES-API- MS m/z: [M + H]+ = 1555.6 | Method 1: Purity: 99% |
| RB- 0005992 (266) | 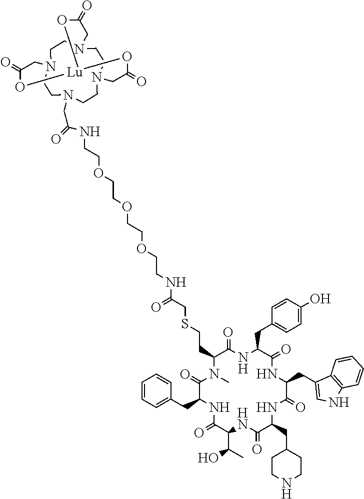 | 1672.6496 | ES-API- MS m/z: [M + H]+ = 1673.6 | Method 1; Purity: 96% |
| RB- 0005994 (267) | 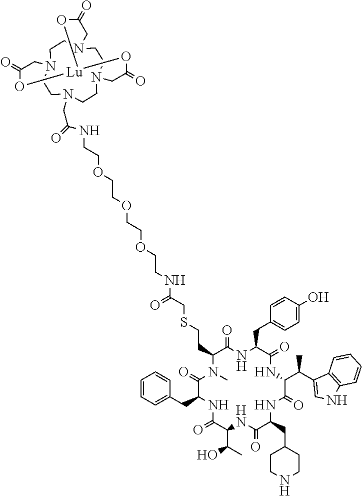 | 1686.6652 | ES-API- MS m/z: [M + H]+ = 1687.6 | Method 1; Purity: 99% |
| RB- 0006009 (268) | 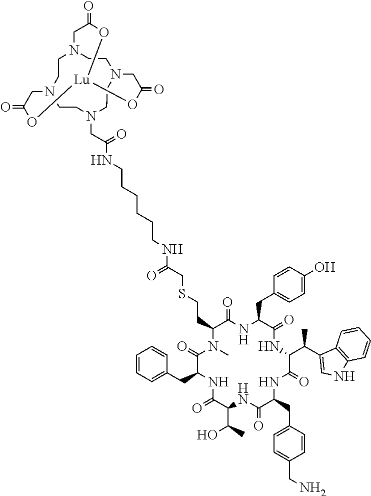 | 1632.6335 | ES-API- MS m/z: [M + H]+ = 1633.6 | Method 1; Purity: 95% |
| RB- 0006011 (269) | 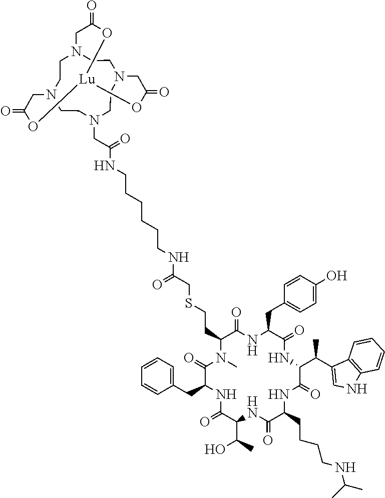 | 1626.6805 | ES-API- MS m/z: [M + H]+ = 1627.6 | Method 1: Purity: 95% |
| RB- 0006013 (270) | 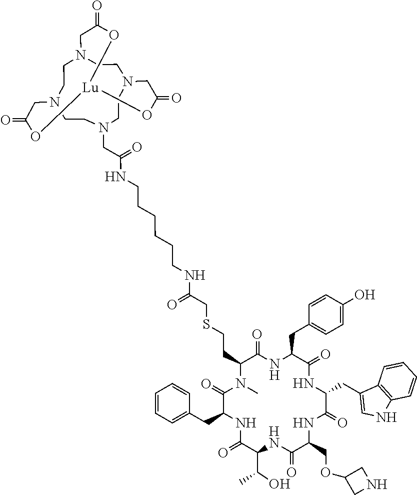 | 1584.5972 | ESI-MS m/z: [M + H]+ = 1585.6 | Method 1: Purity: 96.3% |
| RB- 0006015 (271) | 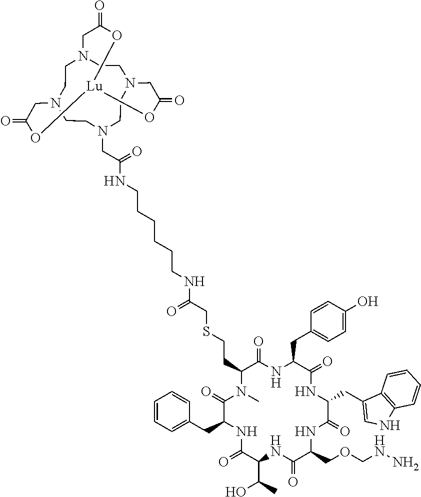 | 1571.6131 | ESI-MS m/z: [M + H]+ = 1572.6 | Method 1; Purity: 95.09% |
| RB- 0006019 (272) | 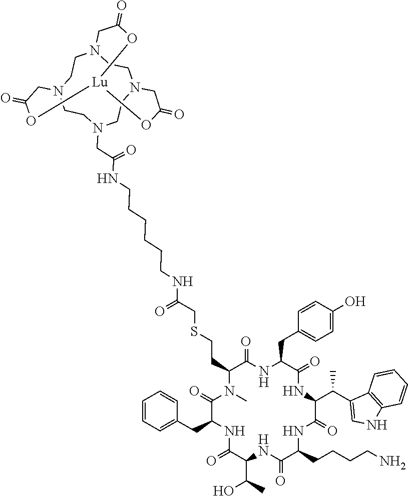 | 1584.6335 | ESI-MS m/z: [M + H]+ = 1585.7 | Method 1; Purity: 96.75% |
| RB- 0006072 (274) | 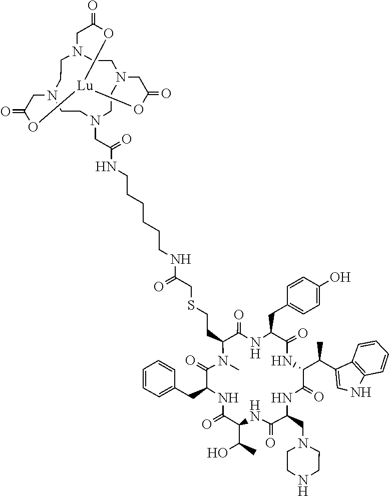 | 1611.6444 | ES-API- MS m/z: [M + H]+ = 1612.6 | Method 1; Purity: 96% |
| RB- 0006073 (275) | 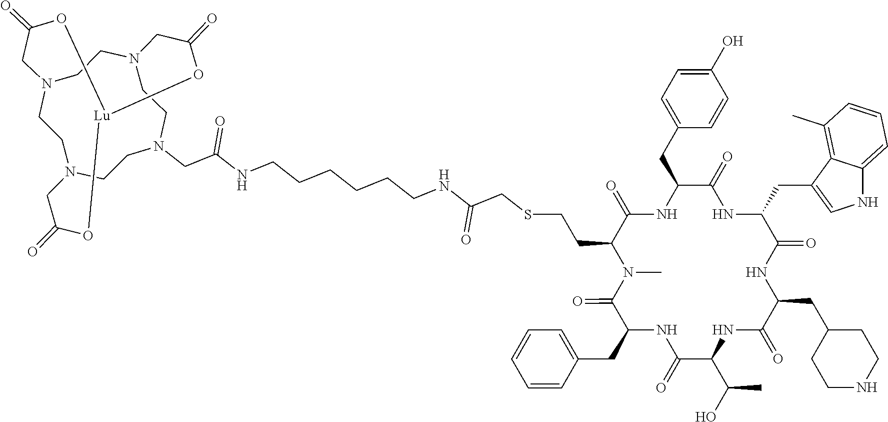 | 1610.6492 | ESI-MS m/z: [M/2 + H]+ = 806.5 | Method 1; Purity: 95% |
| RB- 0006074 (276) | 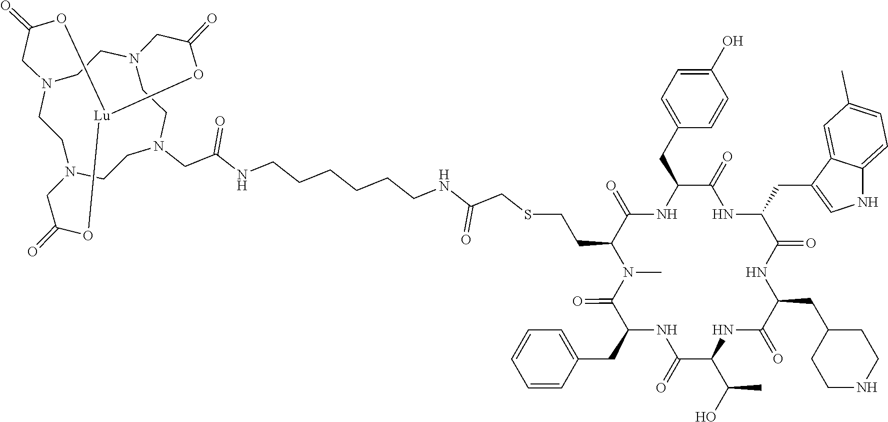 | 1610.6492 | ESI-MS m/z: [M/2 + H]+ = 806.5 | Method 1; Purity: 95% |
| RB- 0006075 (277) | 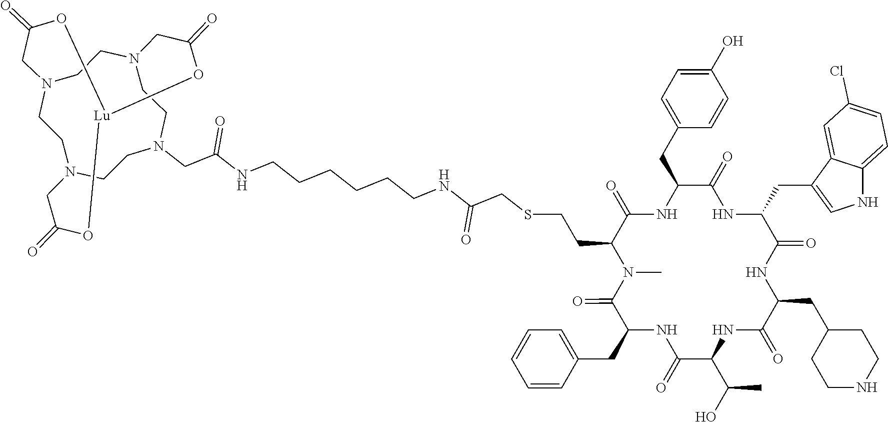 | 1630.5946 | ESI-MS m/z: [M/2 + H]+ = 816.5 | Method 1; Purity: 95% |
| RB- 0006076 (278) | 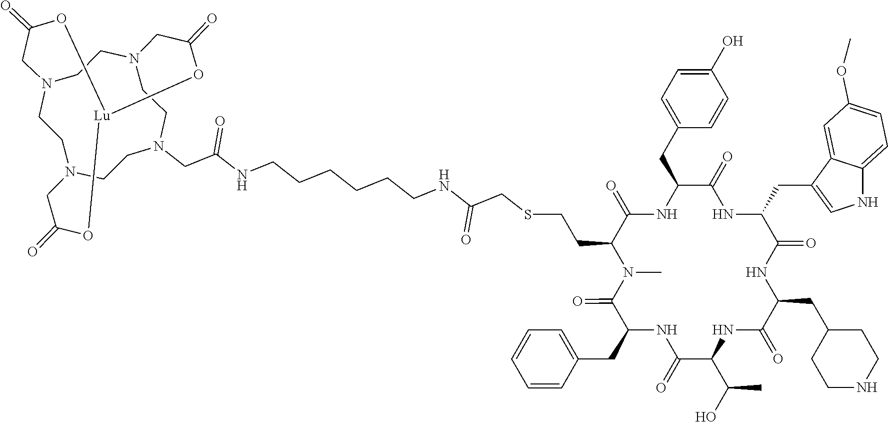 | 1626.6441 | ESI-MS m/z: [M/2 + H]+ = 814.5 | Method 1; Purity: 95% |
| RB- 0006189 (289) | 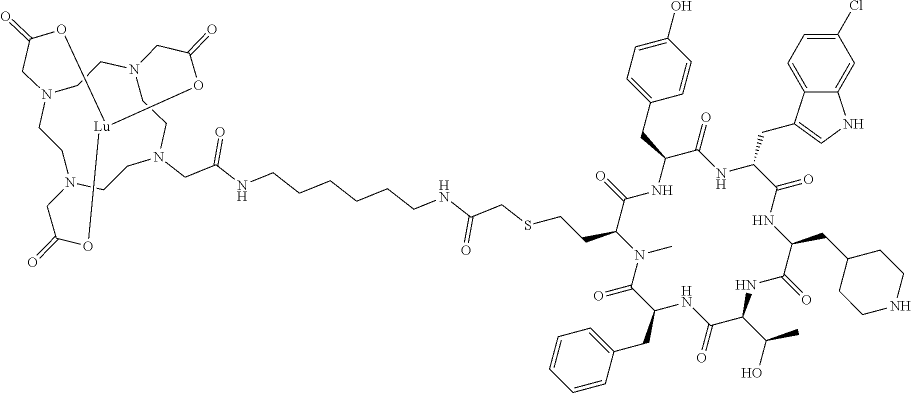 | 1630.5946 | ESI-MS m/z: [M/2 + Na]+ = 838.4 | Method 1; Purity: 95% |
| RB- 0006219 (294) | 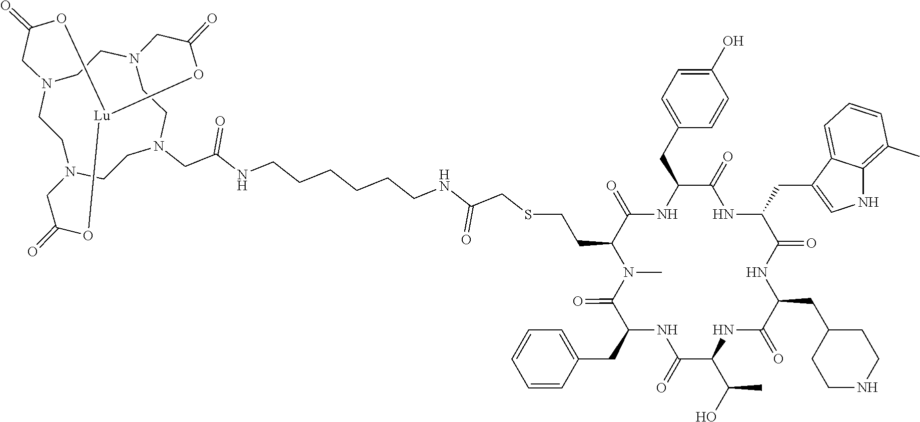 | 1610.6492 | ESI-MS m/z: [M/2 + H]+ = 806.4 | Method 1; Purity: 98% |
| RB- 0006317 (299) | 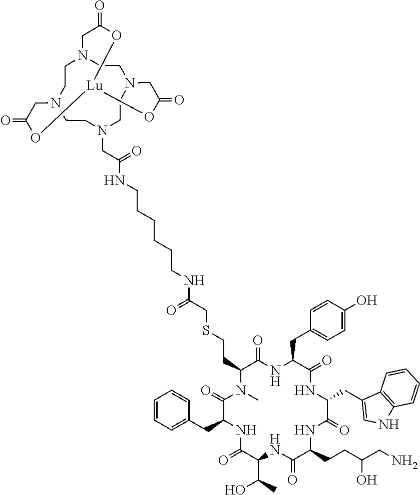 | 1586.6128 | ESI-MS m/z: [M + H]+ = 1587.6 | Method 1; Purity: 96.82% |
| RB- 0006385 (301) | 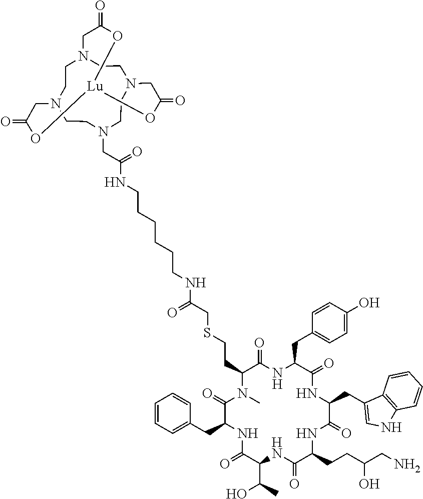 | 1586.6128 | ESI-MS m/z: [M + H]+ = 1587.6 | Method 1; Purity: 97.15% |

### Example A10. Synthesis of Int 9 by Method 2

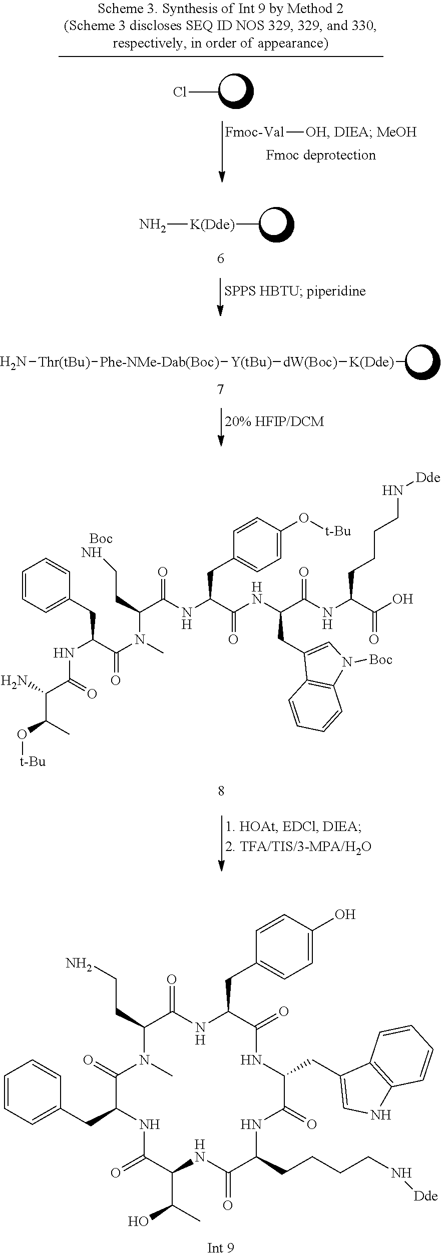

### Method 2:

To the swollen 2-CTC resin was added Fmoc-Lys(Dde)-OH (1 equiv.) and DIEA (4 equiv.) in DCM (5 mL). The mixture was agitated for 2 h under nitrogen. MeOH (1 mL) was then added. The resulting mixture was agitated for 30 min. After the reaction solution was removed through filtration, the resin was washed three times with DMF (10 mL). The Fmoc protecting group was removed via 15 min agitation with 20% piperidine in DMF followed by filtration and washing.

To the peptidyl-resin 6 were added Fmoc-D-Trp(Boc)-OH (3 equiv.), DIEA (6 equiv.) and HBTU (3 equiv.) in DMF (5 mL). The mixture was agitated for 1 h under nitrogen. Next, the reaction solution was removed through filtration, and the resin was washed three times with DMF (10 mL). The Fmoc protecting group was removed via 20 min agitation with 20% piperidine in DMF followed by filtration and washing.

Subsequent amino acids were coupled using Fmoc-protected amino acid (3 equiv.), HBTU (3 equiv.) and DIEA (6 equiv.) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

The peptidyl-resin 7 was washed three times with MeOH and dried under vacuum, the resin was treated with 20% HFIP/DCM for 15 min 3 times. The mixture was concentrated to dryness and then lyophilized to give Int 8

The Int 8 was dissolved in DCM (1 mM) together with EDCI (1.5 equiv.) and HOAt (1.5 equiv.) and DIEA (3 equiv.). The mixture was stirred at 20° C. for 15 h. After the reaction was completed, the mixture was concentrated to dryness. The mixture was then treated with 10 mL TFA cleavage cocktail for 1 hour, and cold isopropyl ether was added. The crude peptide was collected through centrifugation and dried under vacuum. The crude product was purified by preparative HPLC to afford Int 9 as a white solid.

### Example A11. Synthesis of RB-0005717 from Int 9 by Method 2

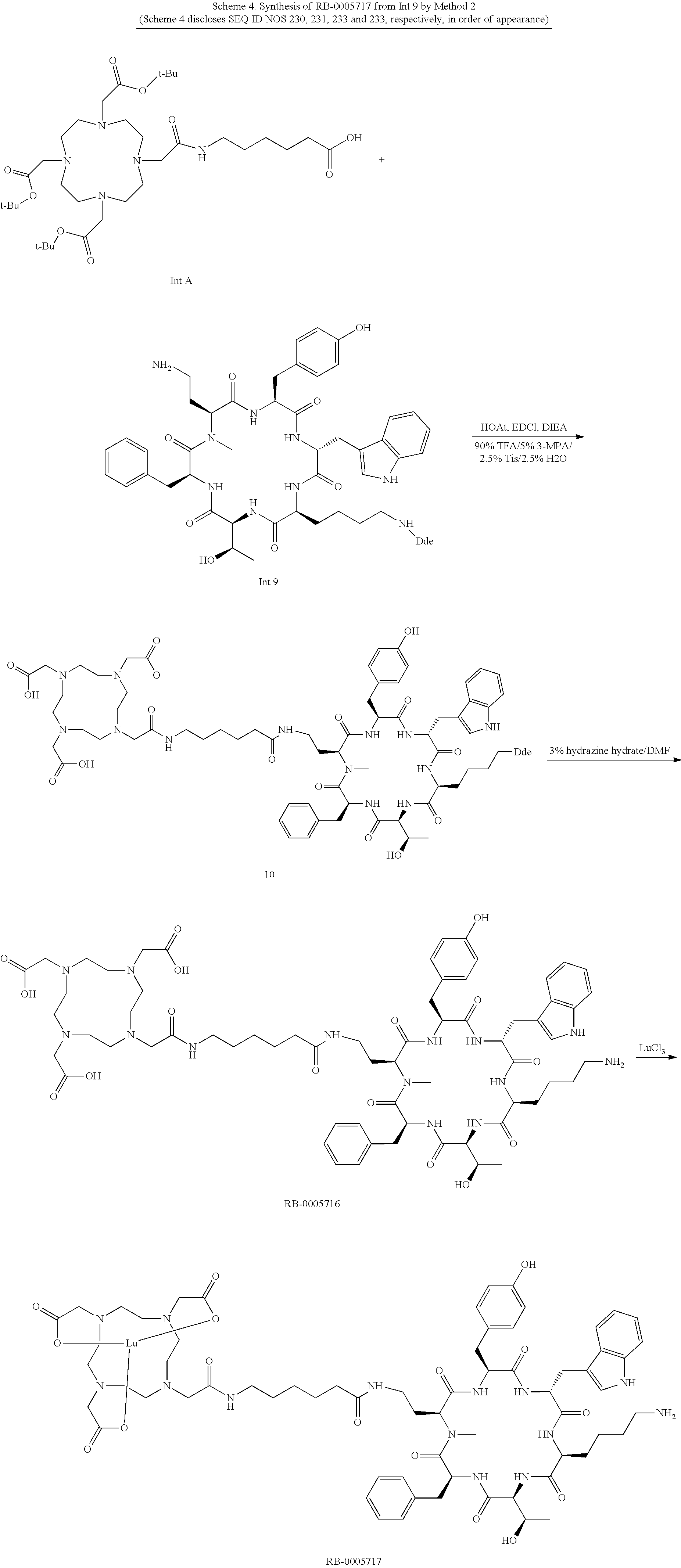

The Int A (1.5 equiv.) and Int 9 (1 equiv.) were dissolved in DMF (30 mL) and added EDCI (1.5 equiv.) and HOAt (1.5 equiv.) and DIEA (3 equiv.). The mixture was stirred at 20° C. for 3 h, then concentrated to dryness. The mixture was treated with 10 mL TFA cleavage cocktail for 2 hour, and cold isopropyl ether was added. The crude peptide was collected through centrifugation and dried under vacuum. The crude product was purified by preparative HPLC to afford Int 10 as a white solid.

The Int 10 was dissolved in 3% hydrazine hydrate/DMF (5 mL), the mixture was stirred at 20° C. for 15 min. After the reaction was completed, the mixture was purified by preparative HPLC to afford RB-0005716 as a white solid.

Following the general protocol for cold labeling of empty-chelator conjugates with Lu, La and Ga metals (See Example A16)—A solution of RB-0005716 (1 equiv.) in 1M Na₂CO₃ aqueous buffer adjusted to pH=5˜6 was added LuCl₃ (5 equiv.). The resulting mixture was stirred at 40° C. for 1 h. After filtration, the crude product was purified by preparative HPLC to afford RB-0005717 as a white solid.

Additional peptide analogues were prepared similarly using this modular synthetic approach outlined in Method 2. The amino acids, fragments, linker, and chelator used in this method vary depending on the peptide sequence. In some instances, the X1 amino acid has a side chain with a terminal amine functionality. In some instances, the chelator is attached directly to X1 or has an extended linker attached to a chelator. In some instances, the chelator is a chelator described herein. In some embodiments, the chelator is NOTA, DOTA, or NODAGA. Additional peptides prepared according to Method 2 are shown in Table 6.

TABLE 6

| Conjugate ID | | Exact mass | Mass observed | Method of |
|---|---|---|---|---|
| (SEQ ID NO) | Structure | (g/mol) | (g/mol) | Synthesis |
| RB-0002327 (1) | 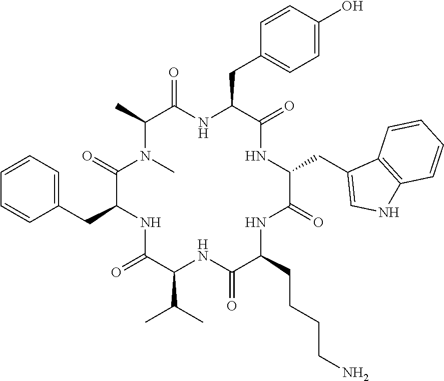 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](C)N(C)C(=O)[C@H](Cc2ccccc2)NC1=O] | 808.4272 | ES-API- MS m/z: [M + H]+ = 809.2 | Method 2; Purity: 98% |
| RB-0002328 (2) | 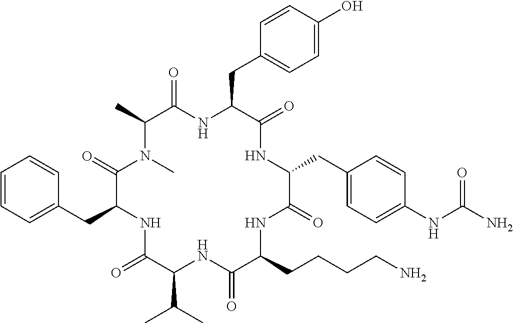 [SMILES: CC(C)[C@@H]1NC(=O)[C@H](CCCCN)NC(=O)[C@@H](Cc2ccc(NC(N)=O)cc2)NC(=O)[C@H](Cc2ccc(O)cc2)NC(=O)[C@H](C)N(C)C(=O)[C@H](Cc2ccccc2)NC1=O] | 827.433 | ES-API- MS m/z: [M + H]+ = 828.4 | Method 2; Purity: 98% |
| RB-0002329 (142) | 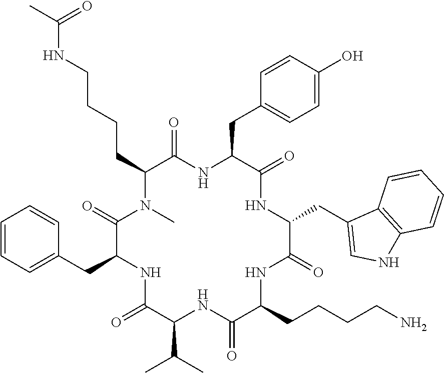 [SMILES: CC(=O)NCCCC[C@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)C)C(=O)N[C@@H](Cc2ccccc2)C(=O)N1C] | 907.4956 | ES-API- MS m/z: [M + H]+ = 908.4 | Method 2; Purity: 98% |
| RB-0002593 (143) | 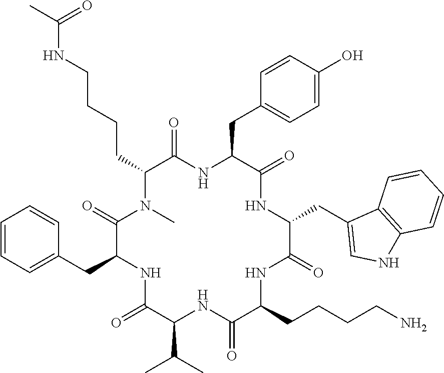 [SMILES: CC(=O)NCCCC[C@@H]1C(=O)N[C@@H](Cc2ccc(O)cc2)C(=O)N[C@H](Cc2c[nH]c3ccccc23)C(=O)N[C@@H](CCCCN)C(=O)N[C@@H](C(C)C)C(=O)N[C@@H](Cc2ccccc2)C(=O)N1C] | 907.4956 | ES-API- MS m/z: [M + H]+ = 908.4 | Method 2; Purity: 99% |
| RB-0002594 (144) | 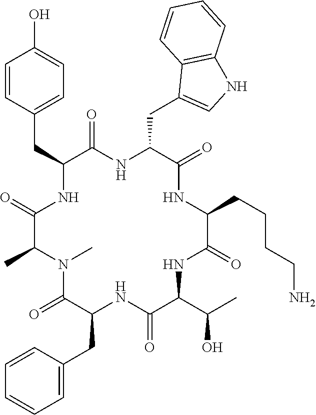 [SMILES: C[C@@H](O)[C@@H]1NC(=O)[C@H](CCCCN)NC(=O)[C@@H](Cc2c[nH]c3ccccc23)NC(=O)[C@H](Cc2ccc(O)cc2)NC(=O)[C@H](C)N(C)C(=O)[C@H](Cc2ccccc2)NC1=O] | 810.4065 | ES-API- MS m/z: [M + H]+ = 811.2 | Method 2; Purity: 99% |
| RB-0002595 (6) | 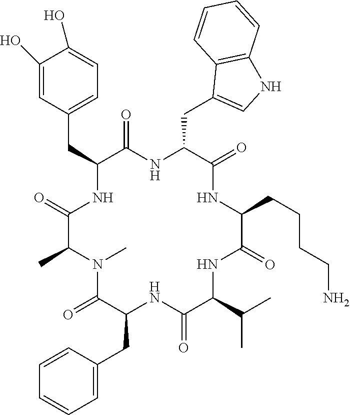 [SMILES: CC(C)[C@@H]1NC(=O)[C@H](CCCCN)NC(=O)[C@@H](Cc2c[nH]c3ccccc23)NC(=O)[C@H](Cc2ccc(O)c(O)c2)NC(=O)[C@H](C)N(C)C(=O)[C@H](Cc2ccccc2)NC1=O] | 824.4221 | ES-API- MS m/z: [M + H]+ = 825.4 | Method 2; Purity: 98.5% |
| RB-0002596 (7) | 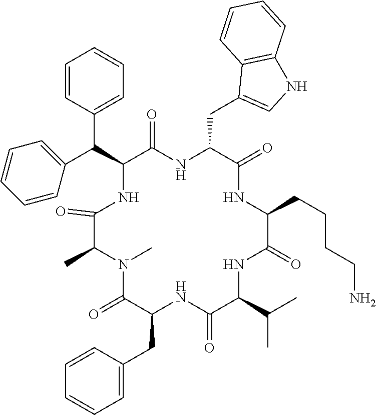 [SMILES: CC(C)[C@@H]1NC(=O)[C@H](CCCCN)NC(=O)[C@@H](Cc2c[nH]c3ccccc23)NC(=O)[C@H](C(c2ccccc2)c2ccccc2)NC(=O)[C@H](C)N(C)C(=O)[C@H](Cc2ccccc2)NC1=O] | 868.4636 | ES-API- MS m/z: [M + H]+ = 869.4 | Method 2; Purity: 98% |
| RB-0002597 (8) | 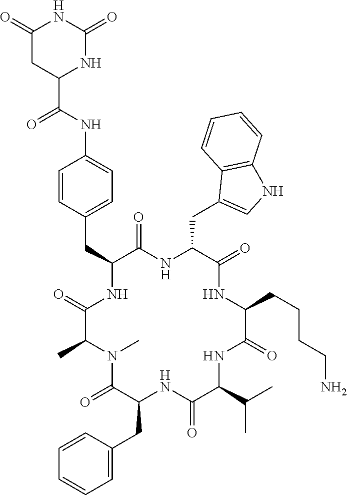 [SMILES: CC(C)[C@@H]1NC(=O)[C@H](CCCCN)NC(=O)[C@@H](Cc2c[nH]c3ccccc23)NC(=O)[C@H](Cc2ccc(NC(=O)C3CC(=O)NC(=O)N3)cc2)NC(=O)[C@H](C)N(C)C(=O)[C@H](Cc2ccccc2)NC1=O] | 947.4654 | ES-API- MS m/z: [M + H]+ = 948.4 | Method 2; Purity: 99% |
| RB-0002670 (145) | 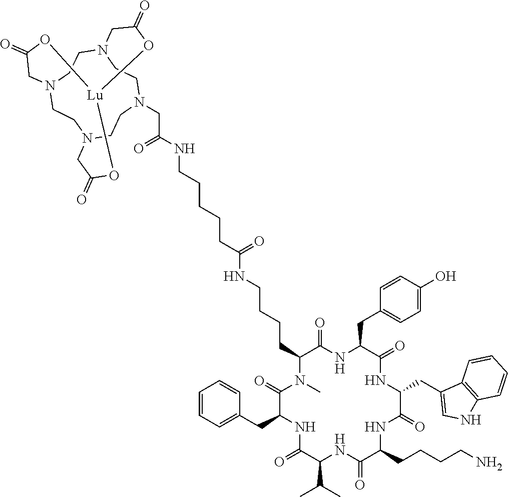 | 1536.6666 | ESI-MS m/z: [M/2 + H]+ = 769.6 | Method 2; Purity: 97.25% |
| RB-0002672 (146) | 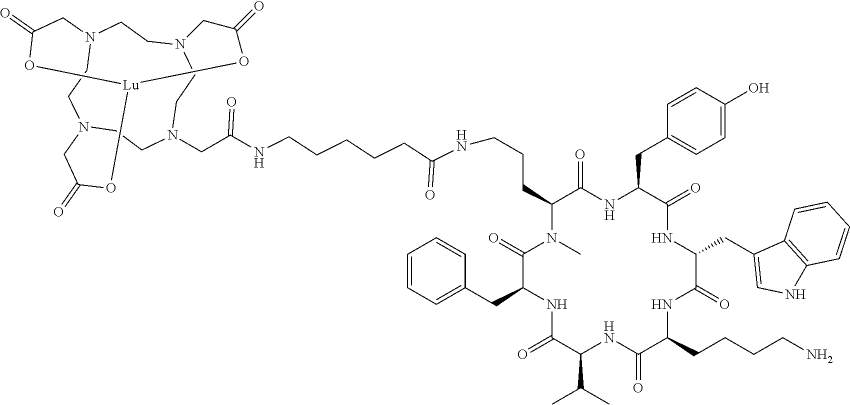 | 1522.6509 | ESI-MS m/z: [M/2 + H]+ = 762.6 | Method 2; Purity: 97.17% |
| RB-0003057 (156) | 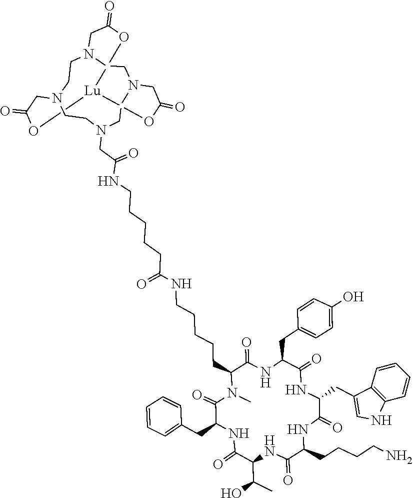 | 1552.6615 | ESI-MS m/z: [M/2 + H]+ = 777.6 | Method 2; Purity: 96.3% |
| RB-0003246 (162) | 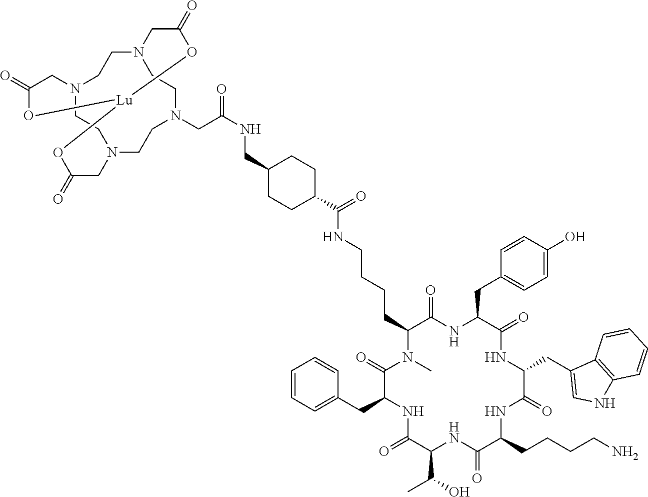 | 1564.6615 | ES-API- MS m/z: [M + H]+ = 1565.6 | Method 2; Purity: 95% |
| RB-0003248 (163) | 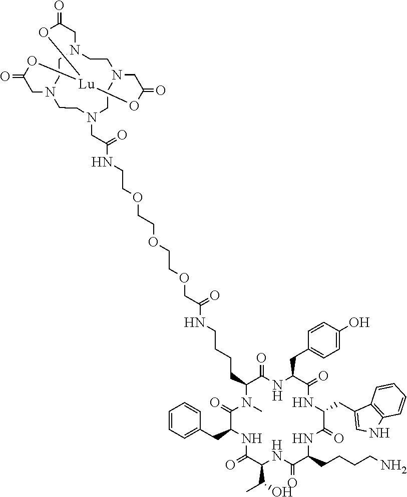 | 1614.6619 | ES-API- MS m/z: [M + H]+ = 1615.6 | Method 2; Purity: 93.8% |
| RB-0003292 (170) | 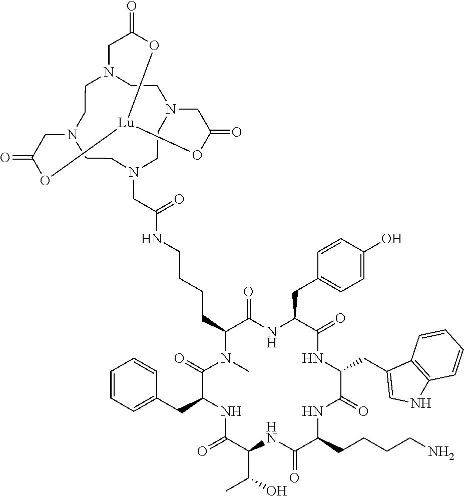 | 1425.5618 | ESI-MS m/z: [M + H]+ = 1426.8 | Method 2; Purity: 96.49% |
| RB-0003294 (171) | 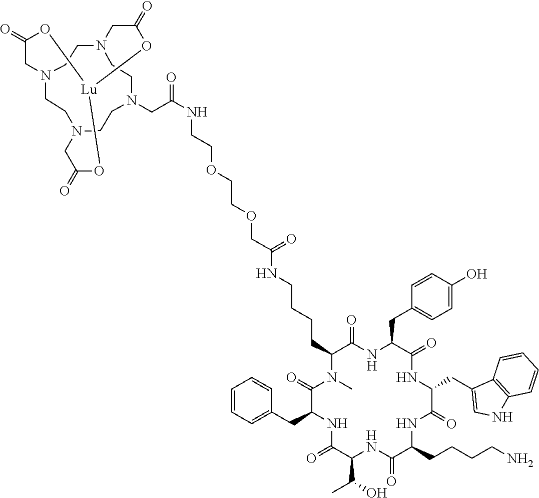 | 1570.6356 | ESI-MS m/z: [M + H]+ = 1571.9 | Method 2; Purity: 96.3% |
| RB-0003296 (172) | 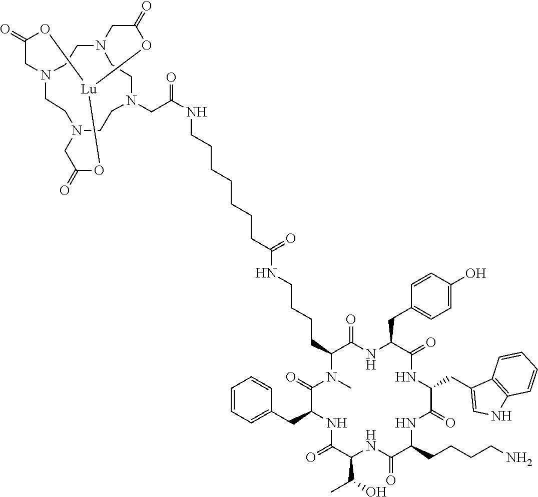 | 1566.6771 | ESI-MS m/z: [M + H]+ = 1567.8 | Method 2; Purity: 97.19% |
| RB-0003298 (173) | 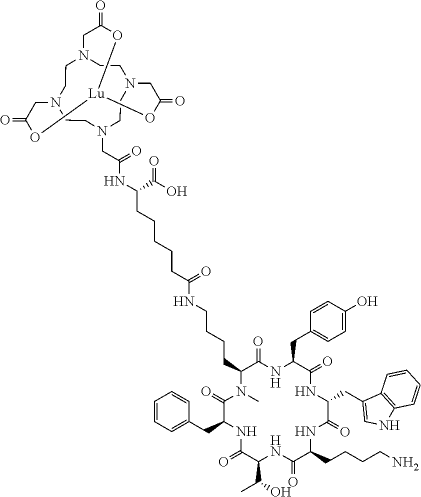 | 1596.6513 | ESI-MS m/z: [M/2 + H]+ = 799.6 | Method 2; Purity: 95.01% |
| RB-0004536 (199) | 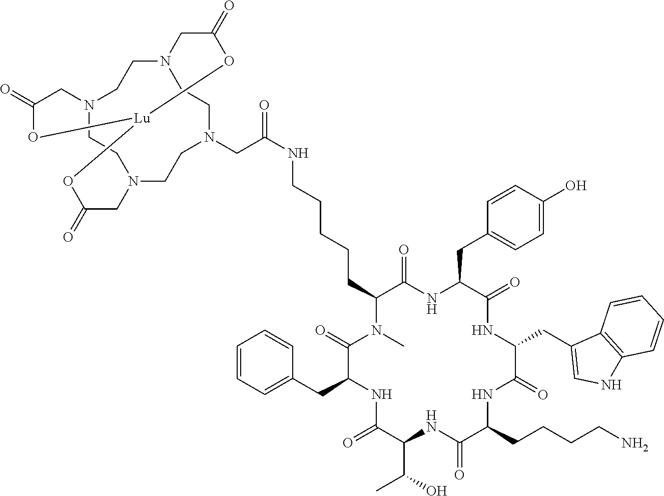 | 1439.5774 | ESI-MS m/z: [M + H]+ = 1440.6 | Method 2; Purity: 97.04% |
| RB-0005717 (233) | 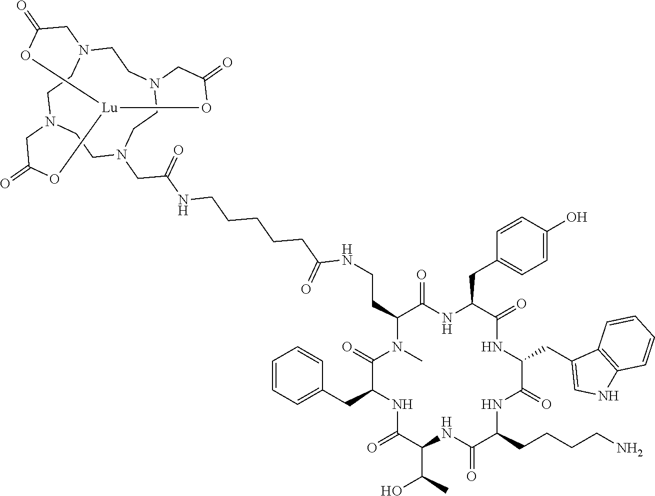 | 1510.6145 | ESI-MS m/z: [M + H]+ = 1511.6 | Method 2; Purity: 97.47% |
| RB-0005728 (234) | 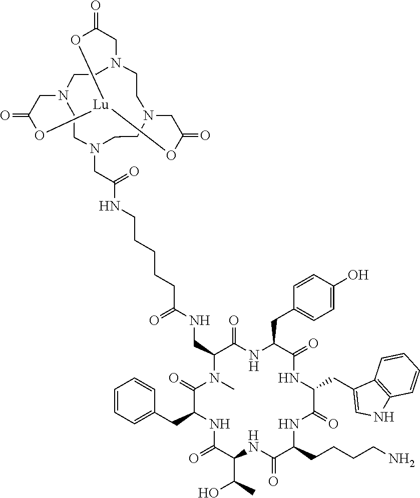 | 1496.5989 | ESI-MS m/z: [M + H]+ = 1497.6 | Method 2; Purity: 96.47% |
| RB-0005730 (235) | 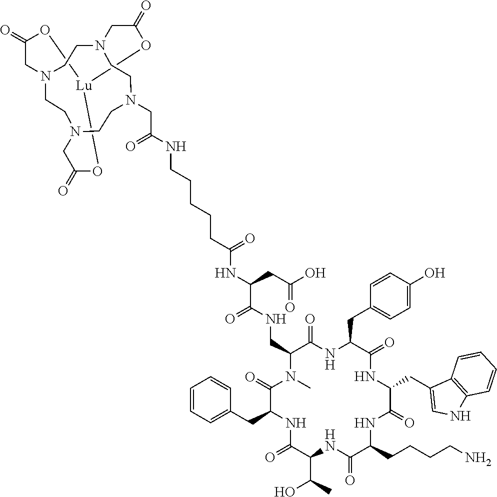 | 1611.6258 | ESI-MS m/z: [M + H]+ = 1612.6 | Method 2; Purity: 97.4% |
| RB-0005732 (236) | 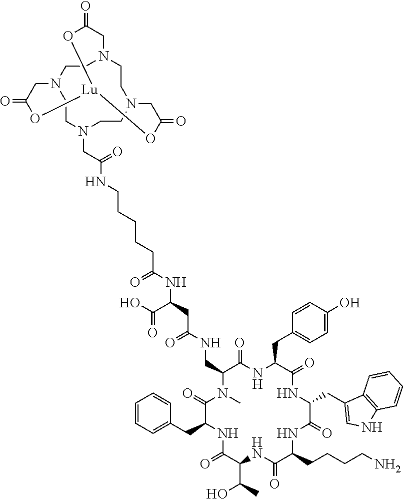 | 1611.6258 | ESI-MS m/z: [M + H]+ = 1612.6 | Method 2; Purity: 97.9% |
| RB-0005734 (237) | 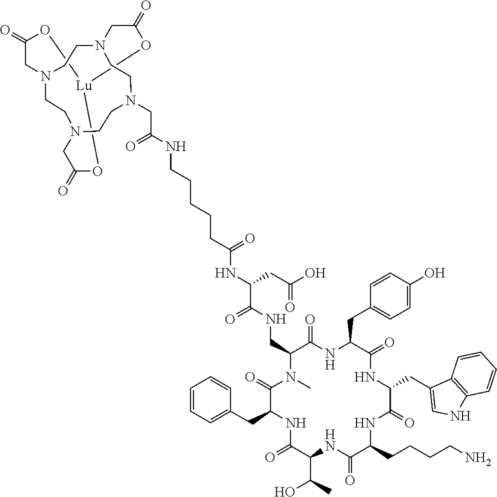 | 1611.6258 | ESI-MS m/z: [M + H]+ = 1612.6 | Method 2; Purity: 96.51% |
| RB-0005736 (238) | 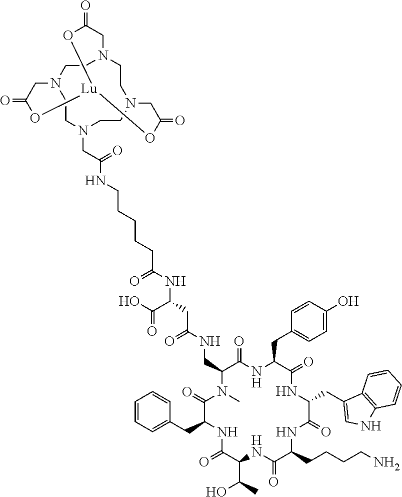 | 1611.6258 | ESI-MS m/z: [M + H]+ = 1612.6 | Method 2; Purity: 97.03% |
| RB-0005797 (244) | 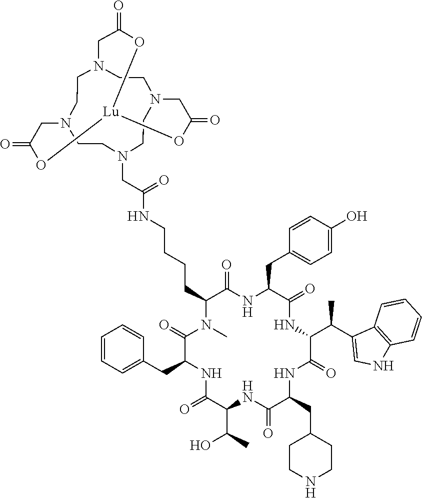 | 1465.5931 | ESI-MS m/z: [M + H]+ = 1446.6 | Method 2; Purity: 97.36% |
| RB-0005852 (251) | 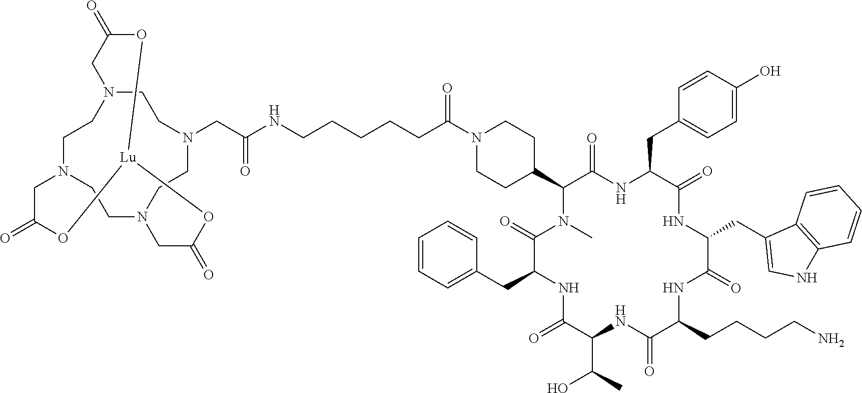 | 1550.6458 | ESI-MS m/z: [M + H]+ = 1551.6 | Method 2; Purity: 95.42% |
| RB-0005854 (252) | 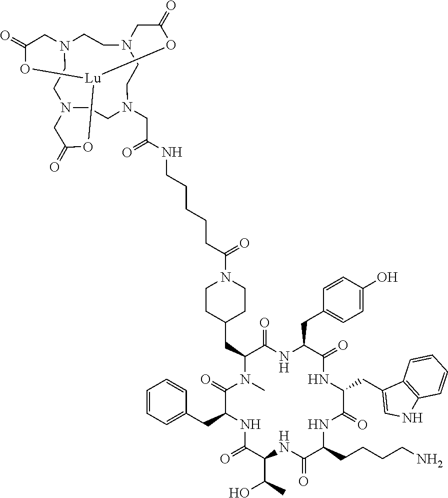 | 1564.6615 | ESI-MS m/z: [M + H]+ = 1565.7 | Method 2; Purity: 96.58% |
| RB-0005983 (262) | 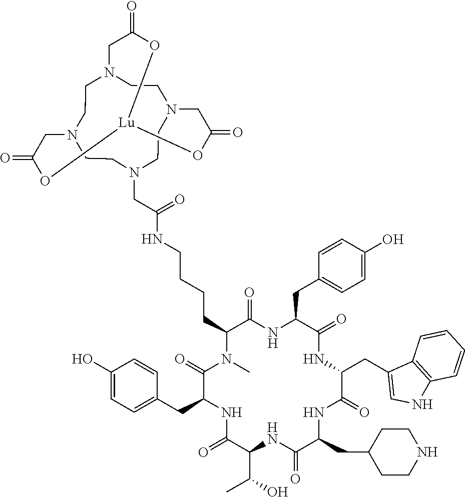 | 1467.5723 | ESI-MS m/z: [M + H]+ = 1468.6 | Method 2; Purity: 99.9% |
| RB-0005984 (263) | 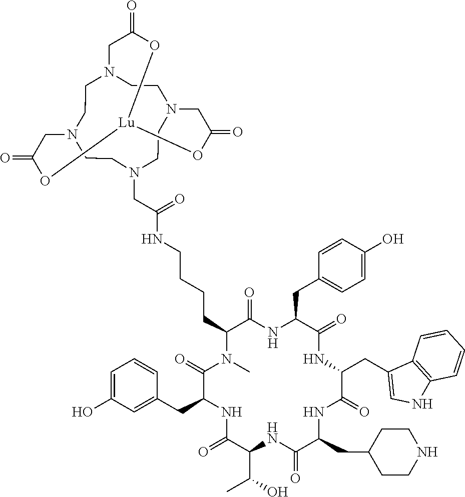 | 1467.5723 | ESI-MS m/z: [M + H]+ = 1468.6 | Method 2; Purity: 99.9% |
| RB-0005985 (264) | 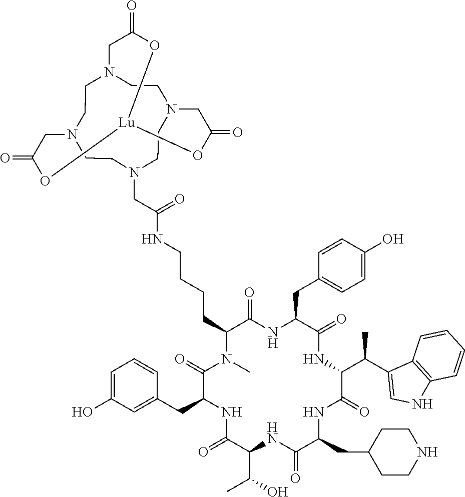 | 1481.588 | ESI-MS m/z: [M + H]+ = 1482.6 | Method 2; Purity: 96.88% |
| RB-0006069 (273) | 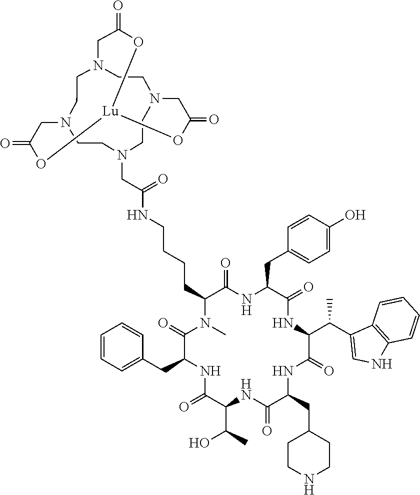 | 1465.5931 | ES-API- MS m/z: [M + H]+ = 1466.5 | Method 2; Purity: 95% |
| RB-0006095 (279) | 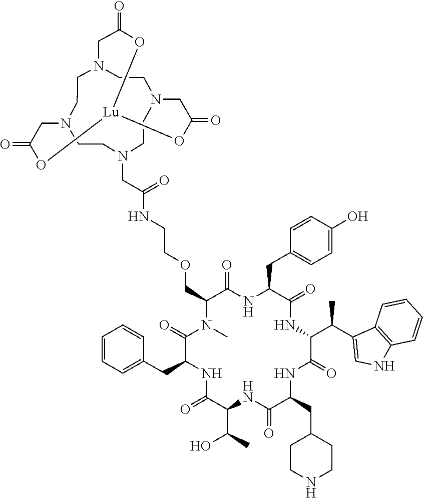 | 1467.5723 | ESI-MS m/z: [M + H]+ = 1468.6 | Method 2; Purity: 97.46% |
| RB-0006128 (280) | 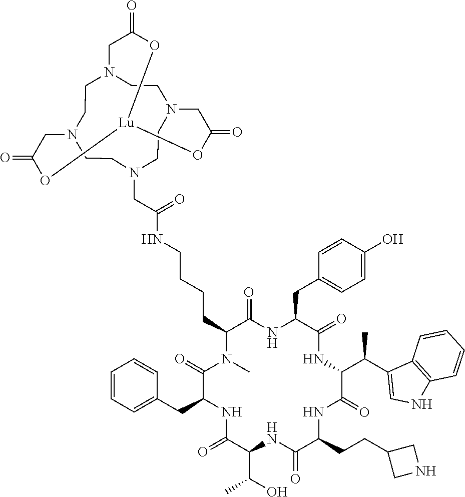 | 1451.5774 | ESI-MS m/z: [M + H]+ = 1452.6 | Method 2; Purity: 96.4% |
| RB-0006139 (281) | 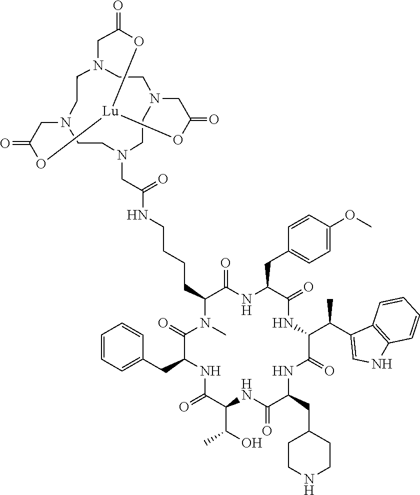 | 1479.6087 | ESI-MS m/z: [M + H]+ = 1480.6 | Method 2; Purity: 97.02% |
| RB-0006158 (282) | 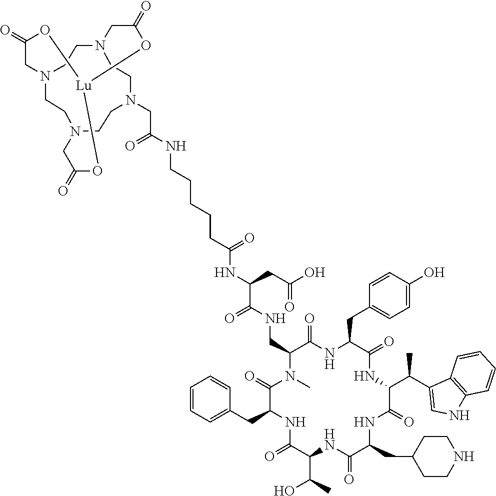 | 1651.6571 | ESI-MS m/z: [M + H]+ = 1652.7 | Method 2; Purity: 95% |
| RB-0006160 (283) | 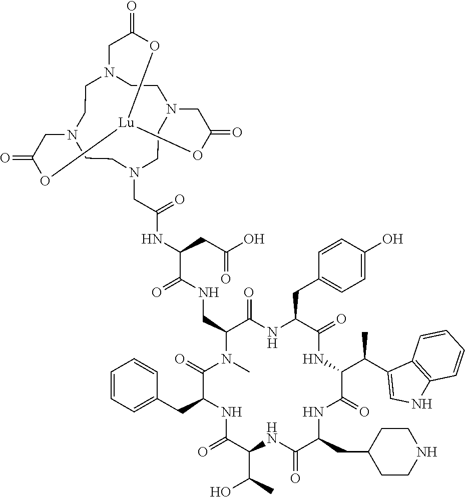 | 1538.573 | ESI-MS m/z: [M + H]+ = 1539.6 | Method 2; Purity: 91.54% |
| RB-0006162 (284) | 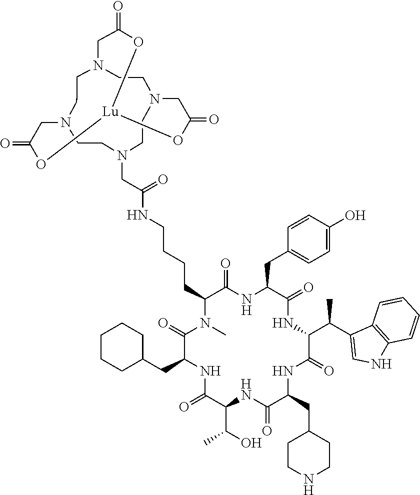 | 1471.64 | ESI-MS m/z: [M + H]+ = 1472.6 | Method 2; Purity: 96.07% |
| RB-0006166 (285) | 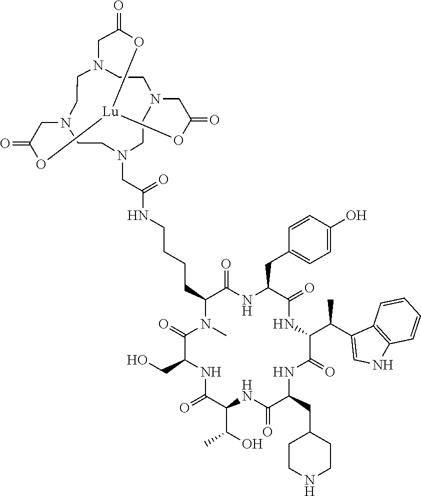 | 1405.5567 | ESI-MS m/z: [M + H]+ = 1406.6 | Method 2; Purity: 97.05% |
| RB-0006170 (286) | 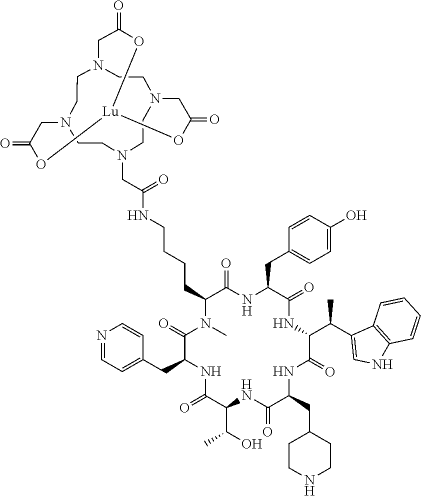 | 1466.5883 | ESI-MS m/z: [M + H]+ = 1467.7 | Method 2; Purity: 97.28% |
| RB-0006174 (287) | 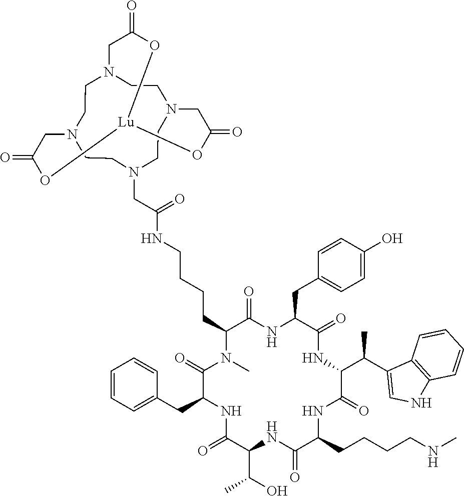 | 1453.5931 | ESI-MS m/z: [M + H]+ = 1454.6 | Method 2; Purity: 96.28% |
| RB-0006176 (288) | 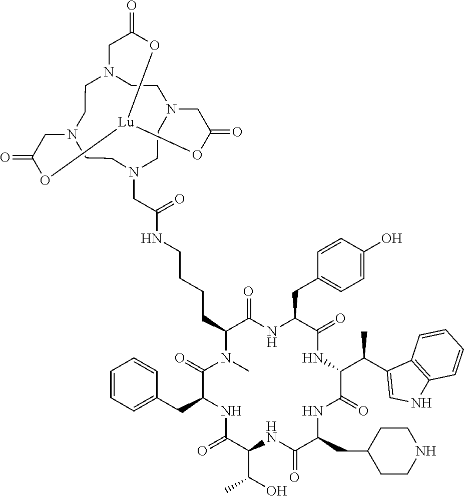 | 1466.5883 | ESI-MS m/z: [M + H]+ = 1467.6 | Method 2; Purity: 96.06% |
| RB-0006209 (290) | 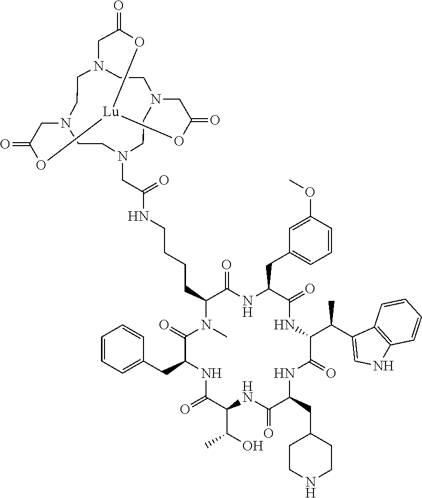 | 1479.6087 | ESI-MS m/z: [M + H]+ = 1480.6 | Method 2; Purity: 97.12% |
| RB-0006213 (291) | 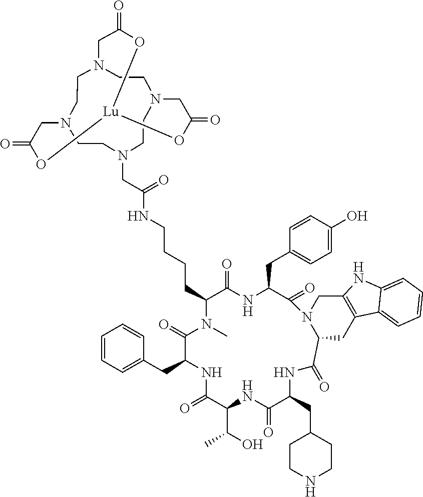 | 1463.5774 | ESI-MS m/z: [M + H]+ = 1464.6 | Method 2; Purity: 95.17% |
| RB-0006215 (292) | 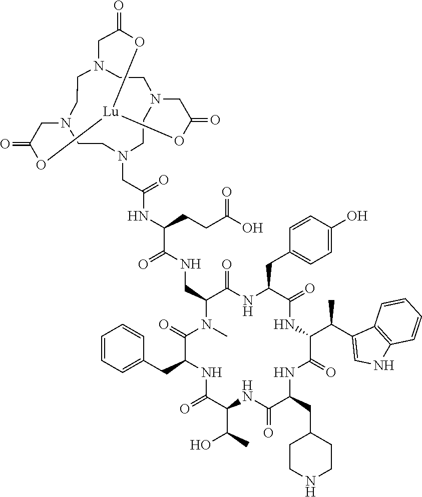 | 1552.5887 | ESI-MS m/z: [M + H]+ = 1553.6 | Method 2; Purity: 92.08% |
| RB-0006217 (293) | 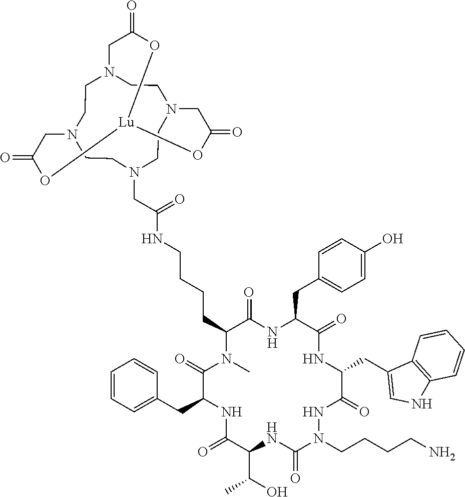 | 1426.557 | ESI-MS m/z: [M + H]+ = 1427.6 | Method 2; Purity: 96.03% |
| RB-0006237 (295) | 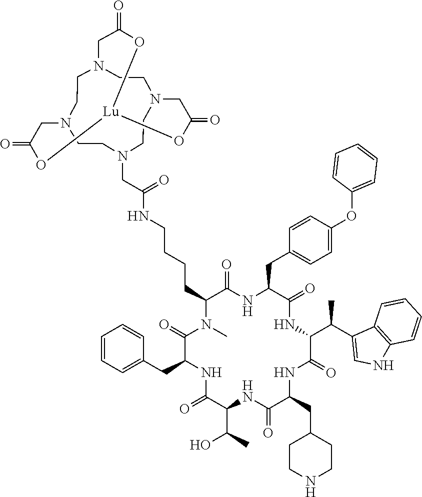 | 1541.6244 | ESI-MS m/z: [M + H]+ = 1542.6 | Method 2; Purity: 97.33% |
| RB-0006239 (296) | 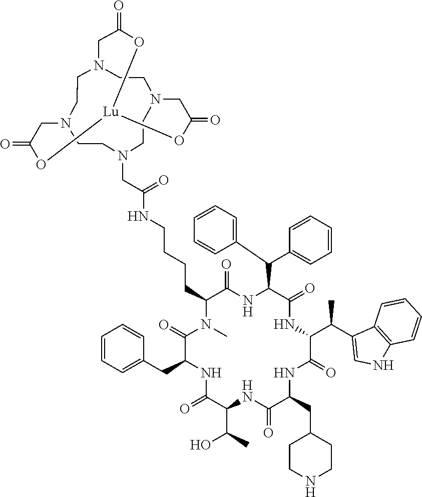 | 1525.6294 | ESI-MS m/z: [M + H]+ = 1526.6 | Method 2; Purity: 97.1% |
| RB-0006285 (297) | 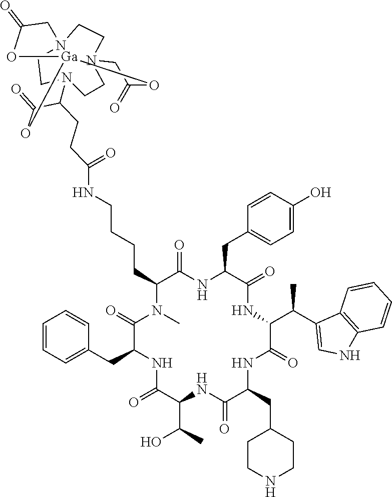 | 1330.5513 | ES-API- MS m/z: [M + H]+ = 1331.6 | Method 2; Purity: 99% |
| RB-0006286 (298) | 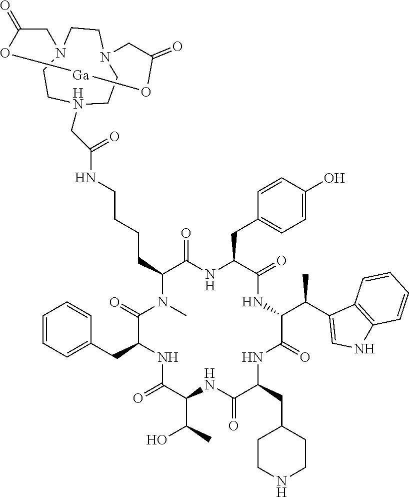 | 1260.5458 | ES-API- MS m/z: [M + H]+ = 1261.6 | Method 2; Purity: 99% |
| RB-0006319 (300) | 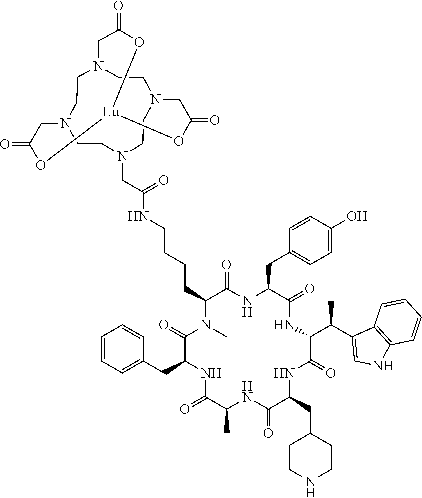 | 1435.5825 | ESI-MS m/z: [M + H]+ = 1436.6 | Method 2; Purity: 96.36% |
| RB-0006387 (302) | 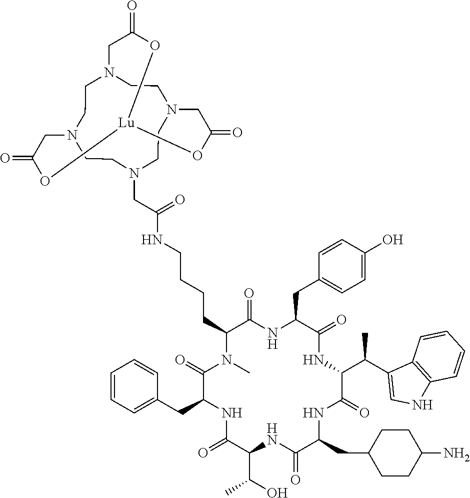 | 1479.6087 | ESI-MS m/z: [M + H]+ = 1480.6 | Method 2; Purity: 96.97% |
| RB-0006389 (303) | 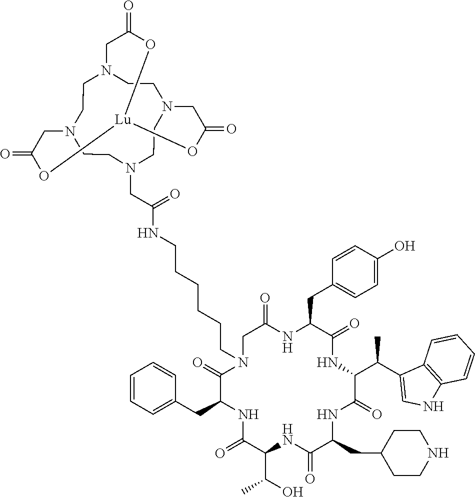 | 1479.6087 | ESI-MS m/z: [M + H]+ = 1480.6 | Method 2; Purity: 98.17% |
| RB-0006397 (307) | 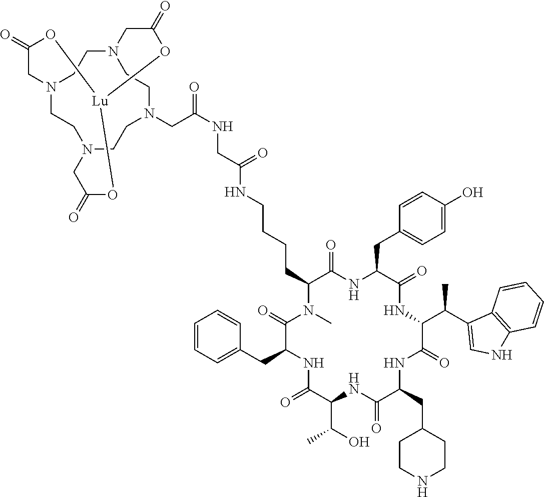 | 1522.6145 | ESI-MS m/z: [M + H]+ = 1523.6 | Method 2; Purity: 97.49% |
| RB-0006400 (308) | 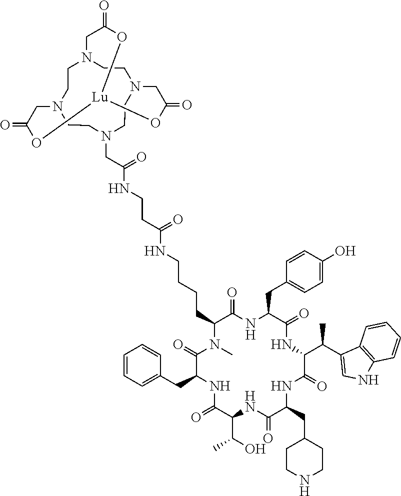 | 1536.6302 | ESI-MS m/z: [M + H]+ = 1537.7 | Method 2; Purity: 97.87% |
| RB-0006403 (309) | 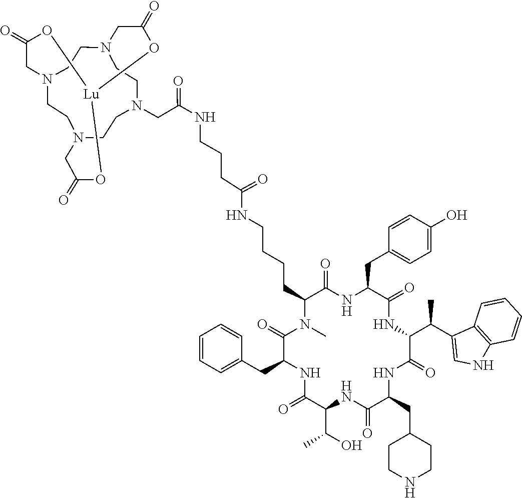 | 1550.6458 | ESI-MS m/z: [M + H]+ = 1551.1 | Method 2; Purity: 97.67% |
| RB-0006406 (310) | 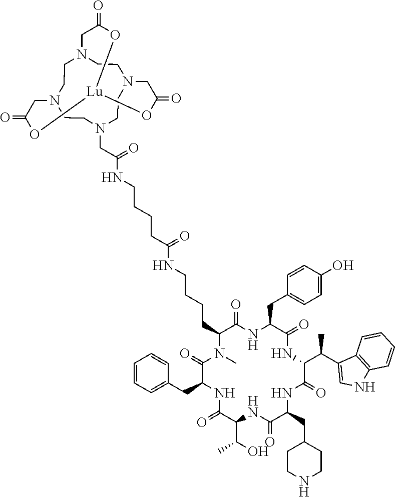 | 1564.6615 | ESI-MS m/z: [M + H]+ = 1565.7 | Method 2; Purity: 97.77% |
| RB-0006414 (311) | 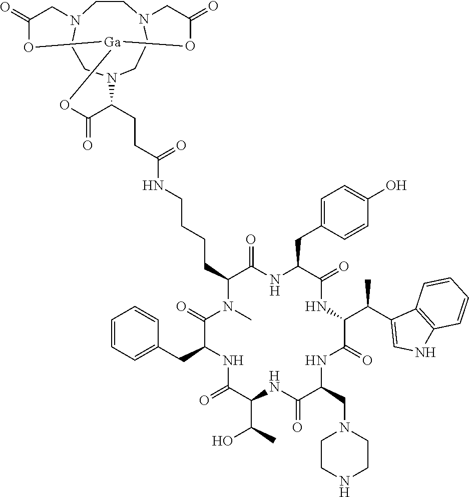 | 1331.5466 | ES-API- MS m/z: [M + H]+ = 1332.5 | Method 2; Purity: 99% |
| RB-0006417 (312) | 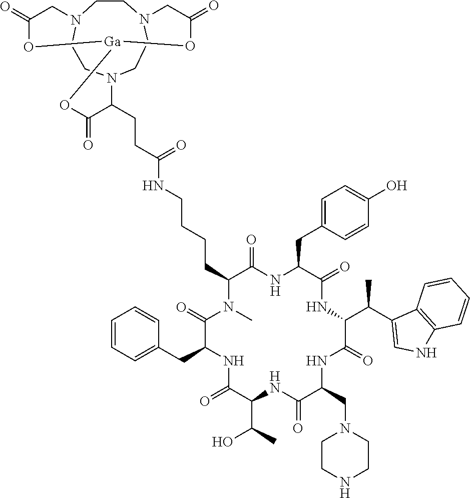 | 1331.5466 | ES-API- MS m/z: [M + H]+ = 1332.4 | Method 2; Purity: 99% |
| RB-0006420 (313) | 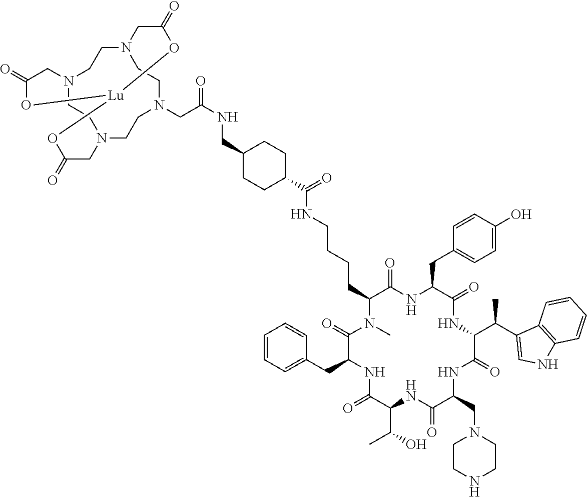 | 1605.688 | ES-API- MS m/z: [M + H]+ = 1606.6 | Method 2; Purity: 98% |
| RB-0006449 (314) | 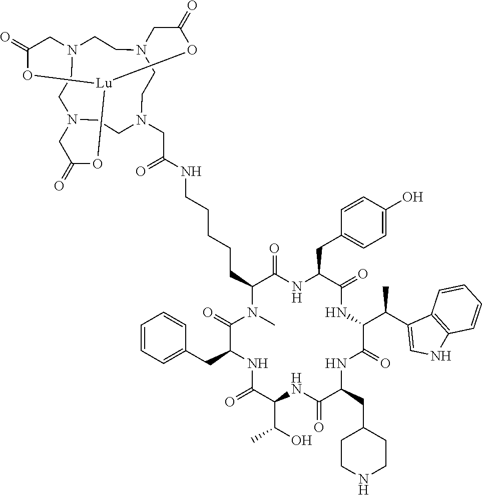 | 1479.6087 | ESI-MS m/z: [M + H]+ = 1480.6 | Method 2; Purity: 96.65% |
| RB-0006452 (315) | 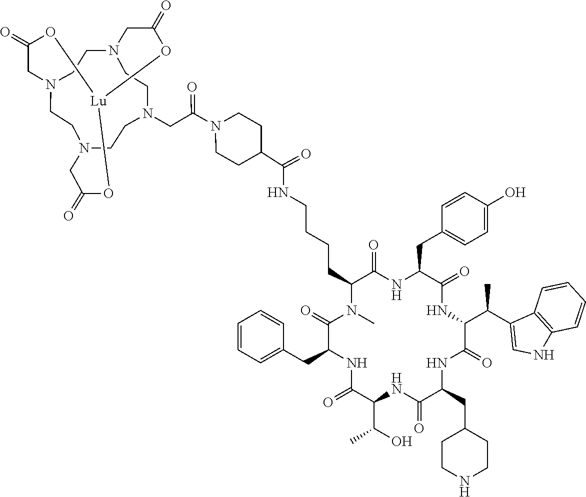 | 1576.6615 | ESI-MS m/z: [M + H]+ = 1577.7 | Method 2; Purity: 99.28% |
| RB-0006455 (316) | 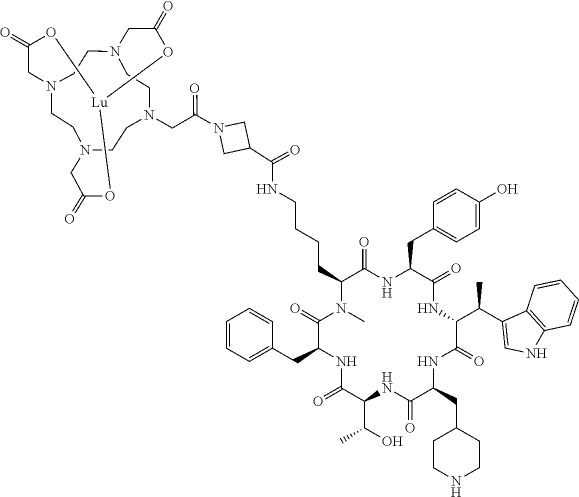 | 1548.6302 | ESI-MS m/z: [M + H]+ = 1549.6 | Method 2; Purity: 98.26% |
| RB-0006458 (317) | 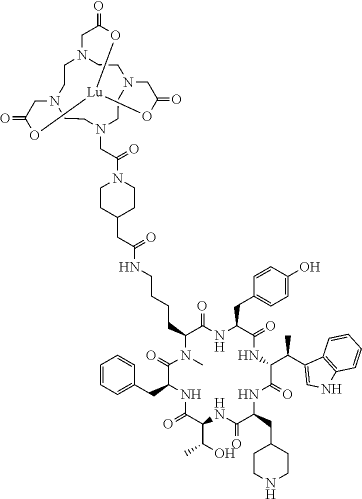 | 1590.6771 | ESI-MS m/z: [M + H]+ = 1591.7 | Method 2; Purity: 97.19% |
| RB-0006461 (318) | 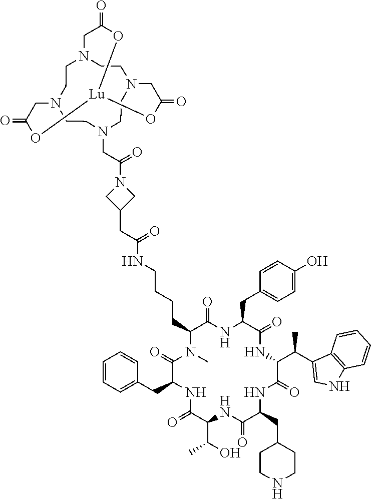 | 1562.6458 | ESI-MS m/z: [M + H]+ = 1563.7 | Method 2; Purity: 99.14% |
| RB-0006464 (319) | 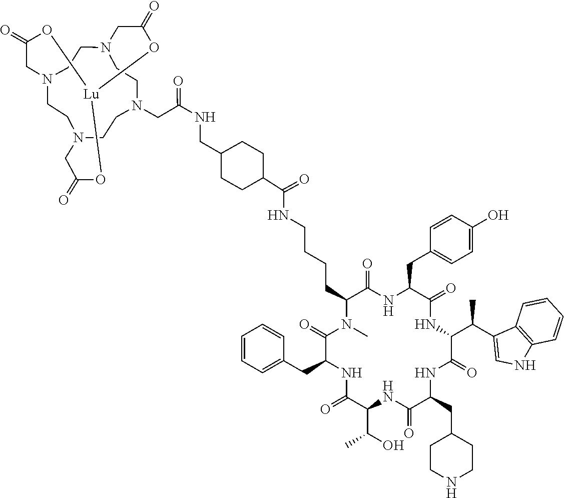 | 1604.6928 | ESI-MS m/z: [M + H]+ = 1605.7 | Method 2; Purity: 96.13% |
| RB-0006470 (320) | 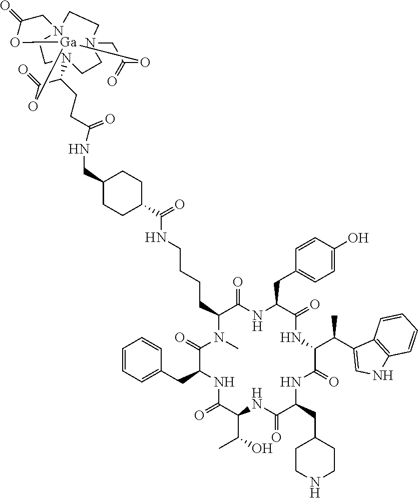 | 1469.651 | ES-API- MS m/z: [M + H]+ = 1470.6 | Method 2; Purity: 99% |
| RB-0006476 (321) | 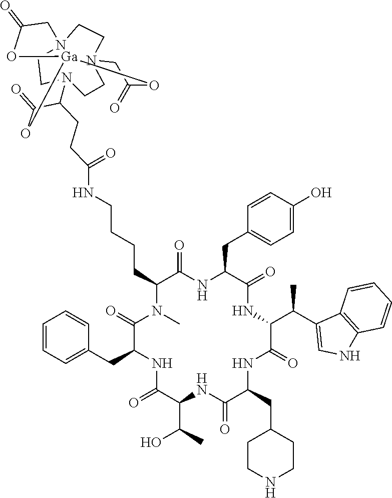 | 1330.5513 | ES-API- MS m/z: [M + H]+ = 1331.6 | Method 2; Purity: >90% |
| RB-0006488 (322) | 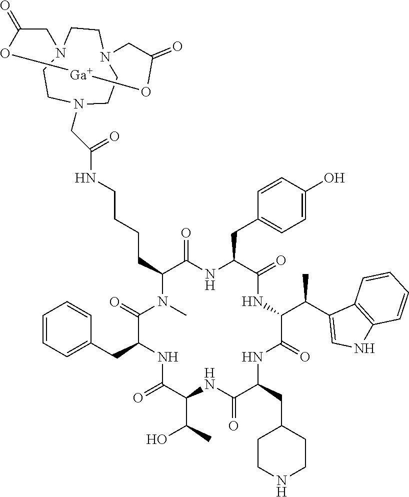 | 1260.5327 | ES-API- MS m/z: [M + H]+ = 1261.6 | Method 2; Purity: >95% |
| RB-0006553 (323) | 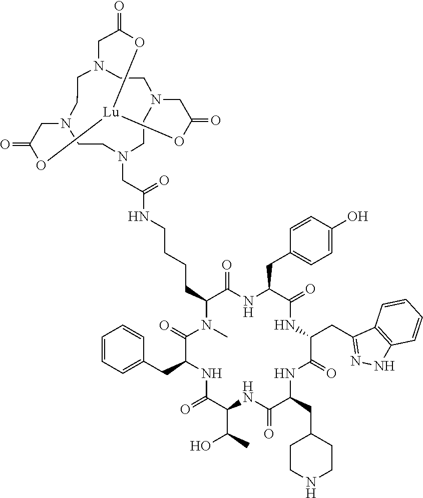 | 1452.5727 | ES-API- MS: m/z [M + H]+ = 1453.8 | Method 2; Purity: 97%% |
| RB-0006558 (324) | 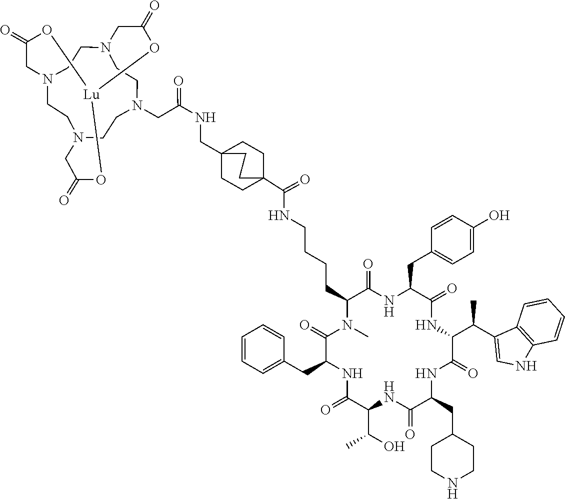 | 1630.7084 | ES-MS: m/z = [M + H]+ = 1631.7 | Method 2; Purity: 97.63% |
| RB-0006579 (325) | 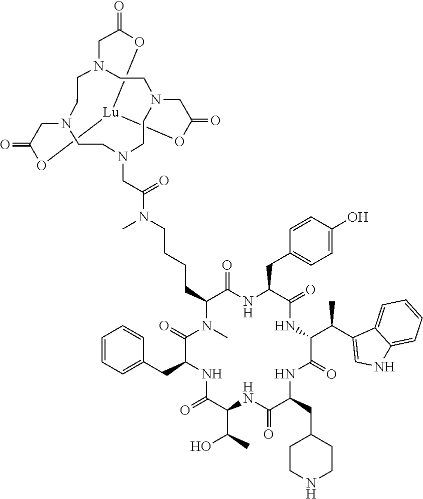 | 1479.6087 | ES-MS: m/z [M + H]+ = 1481.1 | Method 2; Purity: 98.5% |
| RB-0006591 (326) | 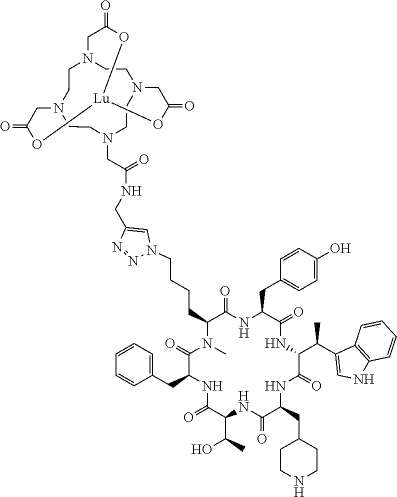 | 1546.6258 | ES-MS: m/z = [M + H]+ = 1548.1 | Method 2; Purity: 99% |

### Example A12. Synthesis of Int 14 by Method 3

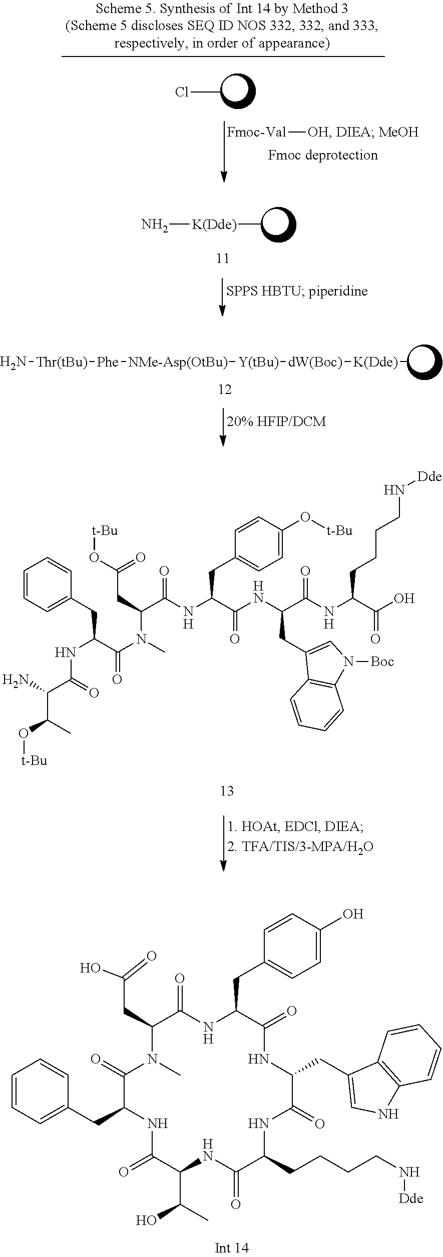

### Method 3:

To the swollen 2-CTC resin were added Fmoc-Lys(Dde)-OH (1 equiv.) and DIEA (4 equiv.) in DCM (5 mL). The mixture was agitated for 2 h under nitrogen. MeOH (1 mL) was then added. The resulting mixture was agitated for 30 min. After the reaction solution was removed through filtration, the resin was washed three times with DMF (10 mL). The Fmoc protecting group was removed via 15 min agitation with 20% piperidine in DMF followed by filtration and washing.

To the peptidyl-resin 11 were added Fmoc-D-Trp(Boc)-OH (3 equiv.), DIEA (6 equiv.) and HBTU (3 equiv.) in DMF (5 mL). The mixture was agitated for 1 h under nitrogen. After the reaction solution was removed through filtration, the resin was washed three times with DMF (10 mL). The Fmoc protecting group was removed via 20 min agitation with 20% piperidine in DMF followed by filtration and washing.

Subsequent amino acids were coupled using Fmoc-protected amino acid (3 equiv.), HBTU (3 equiv.) and DIEA (6 equiv.) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

The peptidyl-resin 12 was washed three times with MeOH and dried under vacuum, the resin was treated with 20% HFIP/DCM for 15 min 3 times. The mixture was concentrated to dryness and then lyophilized to give Int 13 (610 mg)

The Int 13 was dissolved in DCM (1 mM) and added EDCI (1.5 equiv.) and HOAt (1.5 equiv.) and DIEA (3 equiv.). The mixture was stirred at 20° C. for 15 h. After the reaction was completed, the mixture was concentrated to dryness. Then the mixture was treated with 10 mL TFA cleavage cocktail for 1 hour, cold isopropyl ether was added. The crude peptide was collected through centrifugation and dried under vacuum. The crude product was purified by preparative HPLC to afford Int 14 as a white solid.

### Example A13. Synthesis of RB-0005895 from Int 14 by Method

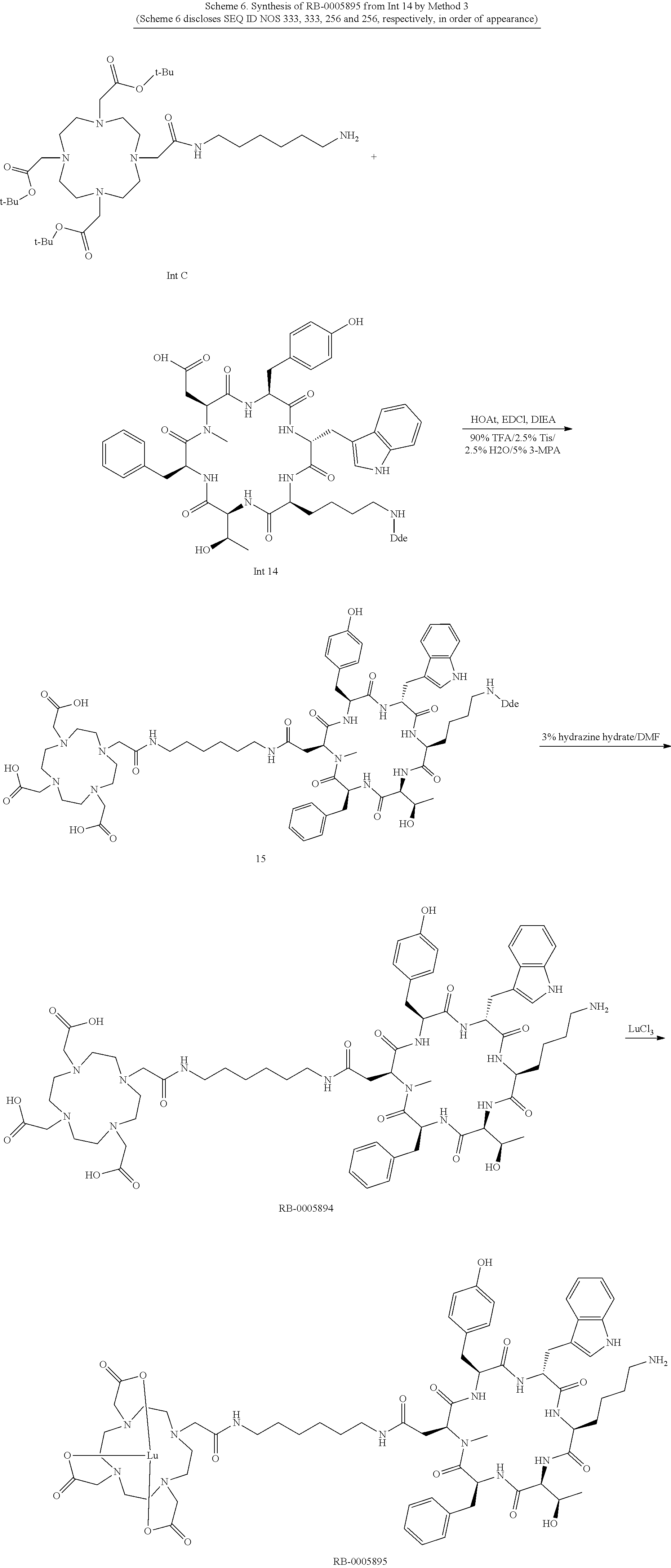

The Int C (1.5 equiv.) and Int 14 (1 equiv.) were dissolved in DMF (30 mL) and added EDCI (1.5 equiv.) and HOAt (1.5 equiv.) and DIEA (3 equiv.). The mixture was stirred at 20° C. for 3 h. After the reaction was completed, it was concentrated to dryness. The mixture was then treated with 10 mL TFA cleavage cocktail for 2 hour, cold isopropyl ether was added. The crude peptide was collected through centrifugation and dried under vacuum. The crude product was purified by preparative HPLC to afford Int 15 as a white solid.

The Int 15 was dissolved in 3% hydrazine hydrate/DMF (5 mL), the mixture was stirred at 20° C. for 15 min. After the reaction was completed, the mixture was purified by preparative HPLC to afford RB-0005894 as a white solid.

Following the general protocol for cold labeling of empty-chelator conjugates with Lu, La and Ga metals (See Example A16)—A solution of RB-0005894 (1 equiv.) in 1M Na₂CO₃ aqueous buffer adjusted to pH=5˜6 was added LuCl₃ (5 equiv.). The resulting mixture was stirred at 40° C. for 1 h. After filtration, the crude product was purified by preparative HPLC to afford RB-0005895 as a white solid.

Additional peptide analogues were prepared similarly using this modular synthetic approach outlined in Method 3. The amino acids, peptoid building blocks, fragments, linker, and chelator used in this method vary depending on the peptide sequence. In some instances, the X1 amino acid has a side chain with a terminal acid functionality. In some instances, the chelator is attached directly to X1 or has an extended linker attached to a chelator. In some instances, the chelator is a chelator described herein. In some embodiments, the chelator is NOTA, DOTA, or NODAGA. Additional peptides prepared according to Method 3 are shown in Table 7.

TABLE 7

| Conjugate | | Exact | Mass | Method |
|---|---|---|---|---|
| ID (SEQ | | mass | observed | of |
| ID NO) | Structure | (g/mol) | (g/mol) | Synthesis |
| RB- 0002678 (149) | 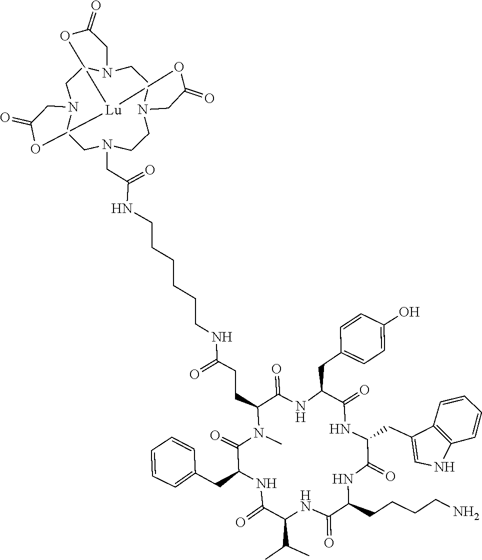 | 1522.6509 | ESI-MS m/z: [M + H]+ = 1523.9 | Method 3; Purity: 97.96% |
| RB- 0002680 (150) | 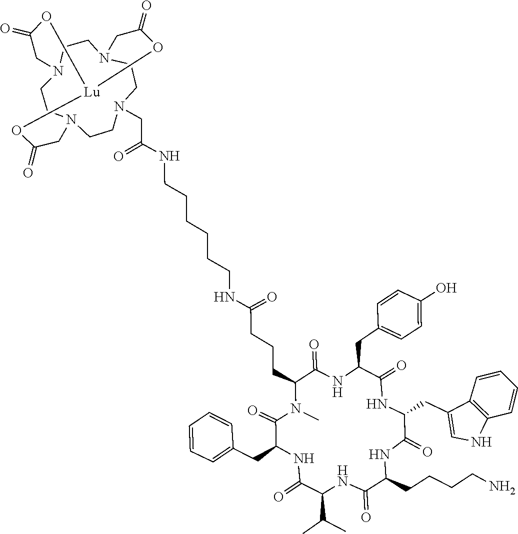 | 1536.6666 | ESI-MS m/z: [M/2 + H]+ = 769.6 | Method 3; Purity: 95.31% |
| RB- 0003055 (155) | 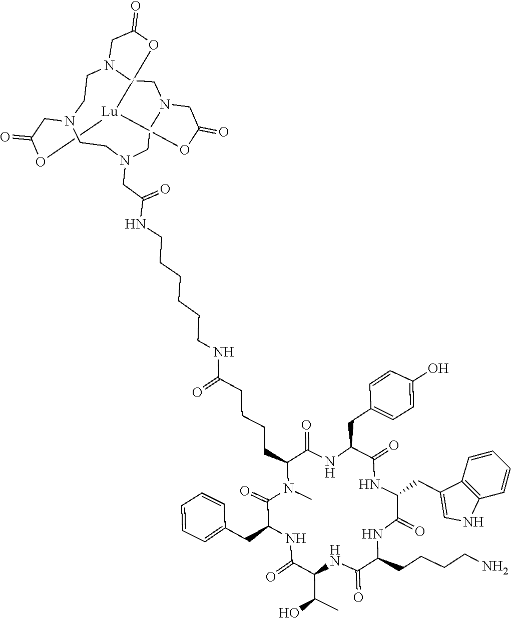 | 1552.6615 | ESI-MS m/z: [M + H]+ = 1553.8 | Method 3; Purity: 95.79% |
| RB- 0005895 (256) | 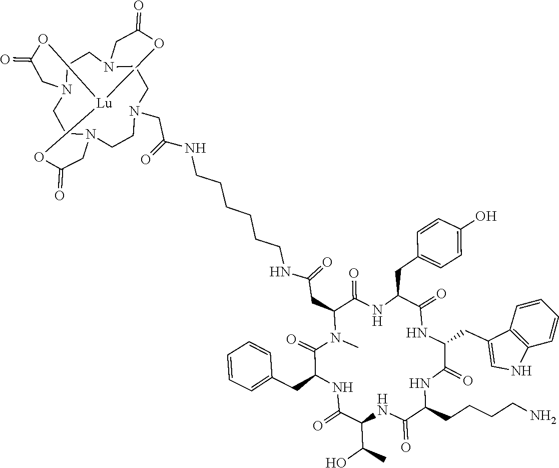 | 1510.6145 | ESI-MS m/z: [M + H]+ = 1511.6 | Method 3; Purity: 96.49% |

### Example A14. Synthesis of Int 19 by Method 4

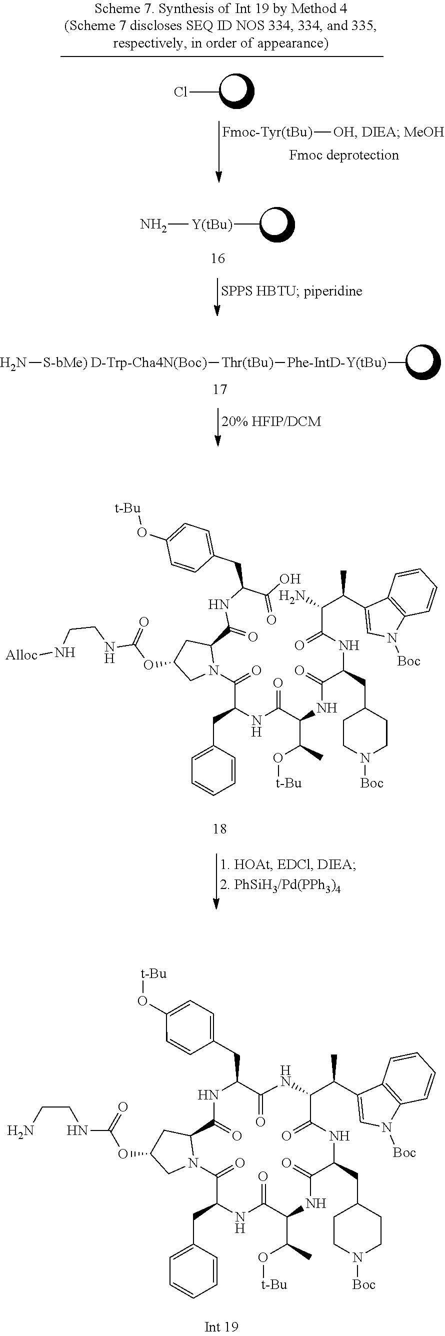

To the swollen 2-CTC resin were added Fmoc-Tyr(tBu)-OH (1 equiv.) and DIEA (4 equiv.) in DCM (5 mL). The mixture was agitated for 2 h under nitrogen. MeOH (1.00 mL) was then added. The resulting mixture was agitated for 30 min. After the reaction solution was removed through filtration, the resin was washed three times with DMF (10 mL). The Fmoc protecting group was removed via 15 min agitation with 20% piperidine in DMF followed by filtration and washing.

To the peptidyl-resin 16 were added Int D (2 equiv.), DIEA (4 equiv.) and HBTU (4 equiv.) in DMF (5 mL). The mixture was agitated for 1 h under nitrogen. After the reaction solution was removed through filtration, the resin was washed three times with DMF (10 mL). The Fmoc protecting group was removed via 20 min agitation with 20% piperidine in DMF followed by filtration and washing.

Subsequent amino acids were coupled using Fmoc-protected amino acid (3 equiv.), HBTU (3 equiv.) and DIEA (6 equiv.) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

After the resin was washed three times with MeOH and dried under vacuum, the resin was treated with 20% HFIP/DCM for 15 min 3 times. The mixture was concentrated to dryness and then lyophilized to give Int 18.

Int 18 was dissolved in DMF and added EDCI (1.5 equiv.), HOAt (1.5 equiv.) and DIEA (3 equiv.). The mixture was stirred at 20° C. for 15 h. After the reaction was completed, the mixture was concentrated to dryness. The mixture was then dissolved in DCM (20 mL) together with PhSiH3 (10 equiv.) and Pd(PPh3)4 (0.1 equiv.). The mixture was stirred at 25° C. for 0.5 hours. After the reaction was completed, the mixture was concentrated to dryness and purified by preparative HPLC to afford Int 19 as a white solid.

### Example A15. Synthesis of RB-0006395 from Int 19 by Method 4

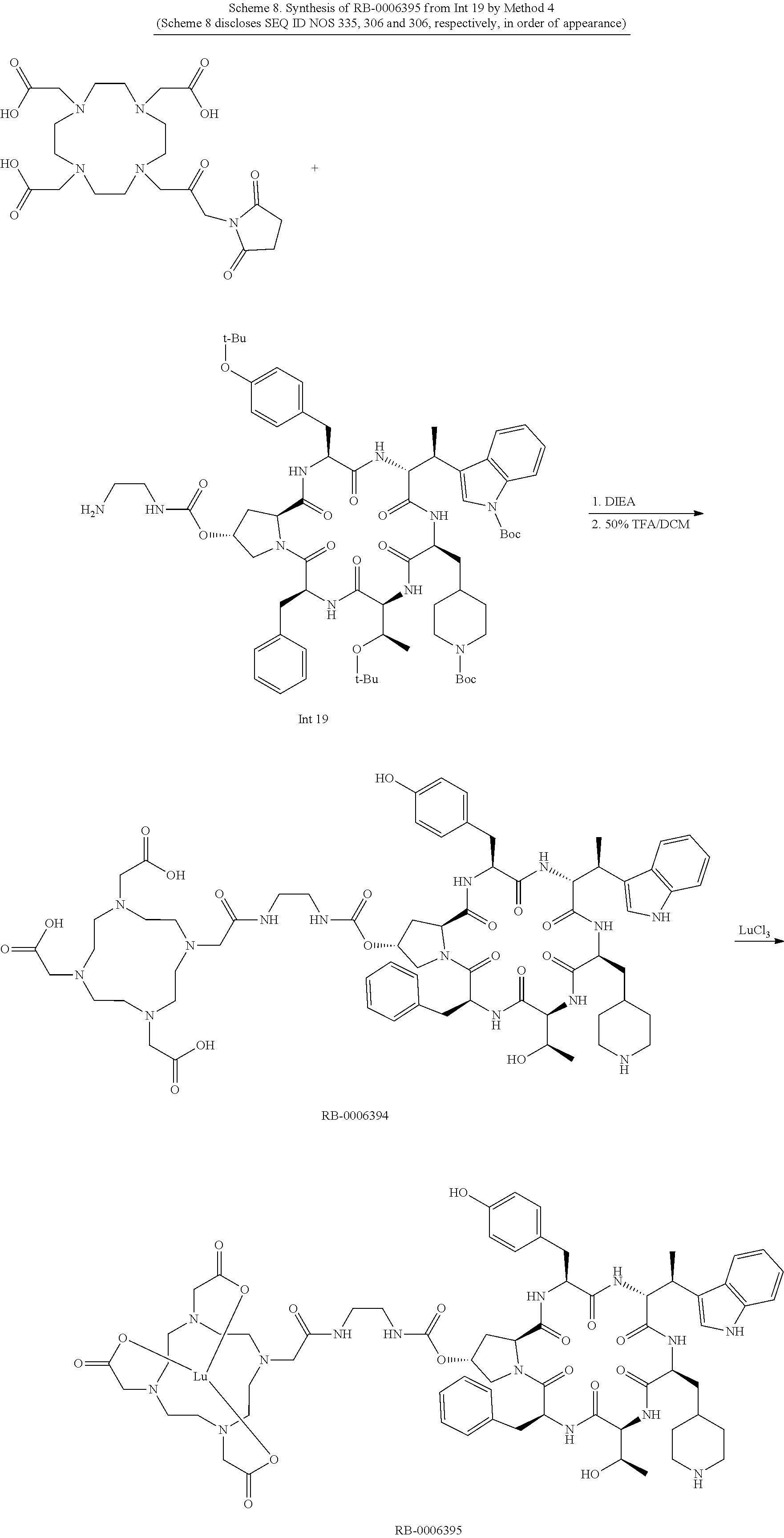

The Int 19 (SEQ ID NO: 335; 1 equiv.) and DOTA-NHS (1.5 equiv.) were dissolved in DMF (20 mL) and added DIEA (5 equiv.). The mixture was stirred at 25° C. for 0.5 hour. After the reaction was completed, the mixture was concentrated to dryness.

The mixture was treated with 20 mL TFA cleavage cocktail for 1 hour, then concentrated to 2 mL, and cold isopropyl ether was added. The crude peptide was collected through centrifugation and dried under vacuum. The crude product was purified by preparative HPLC to afford RB-0006394 (SEQ ID NO: 306) as a white solid.

Following the general protocol for cold labeling of empty-chelator conjugates with Lu, La and Ga metals (See Example A16)—A solution of RB-0006394 (SEQ ID NO: 306; 1 equiv.) in 1M Na₂CO₃ aqueous buffer adjusted to pH=5˜6 was added LuCl₃ (5 equiv.). The resulting mixture was stirred at 40° C. for 1 h. After filtration, the crude product was purified by preparative HPLC to afford RB-0006395 (SEQ ID NO: 306) as a white solid.

Additional peptide analogues were prepared similarly using this modular synthetic approach outlined in Method 4. The amino acids, fragments, linker, and chelator used in this method vary depending on the peptide sequence. In some instances, the X1 amino acid is a proline derivative. In some instances, the chelator is attached directly to X1 or has an extended linker attached to a chelator. In some instances, the chelator is a chelator described herein. In some embodiments, the chelator is NOTA, DOTA, or NODAGA. Additional peptides prepared according to Method 4 are shown in Table 8.

TABLE 8

| Conjugate | | Exact | Mass | Method |
|---|---|---|---|---|
| ID (SEQ | | mass | observed | of |
| ID NO) | Structure | (g/mol) | (g/mol) | Synthesis |
| RB- 0006391 (304) | 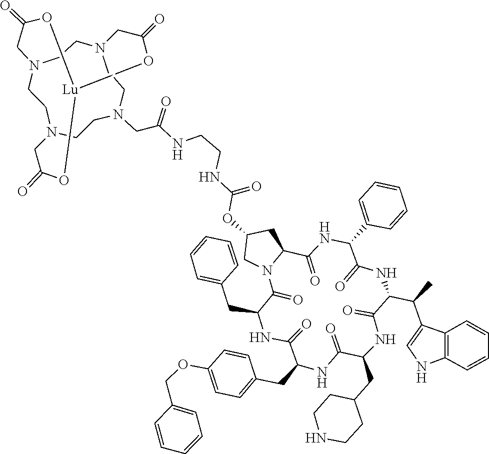 | 1644.6302 | ESI-MS m/z: [M + H]+ = 1645.6 | Method 4; Purity: 95.06% |
| RB- 0006393 (305) | 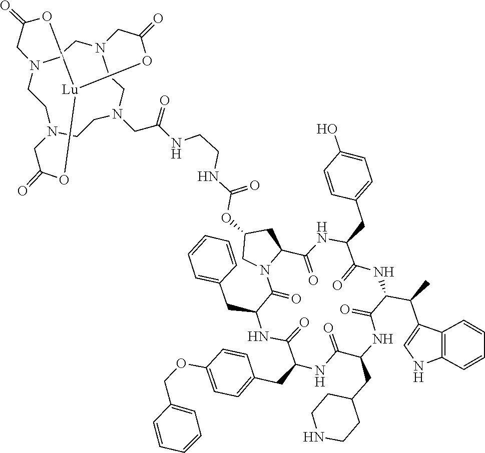 | 1674.6407 | ESI-MS m/z: [M + H]+ = 1675.7 | Method 4; Purity: 99.41% |
| RB- 0006395 (306) | 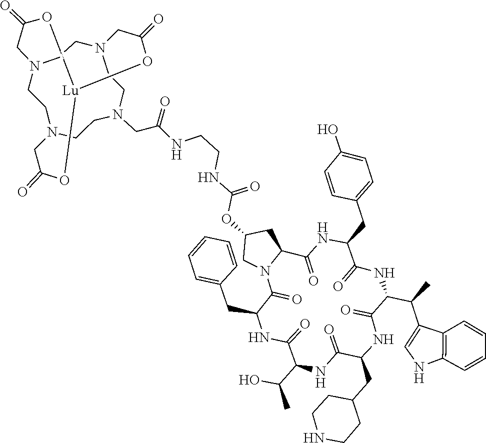 | 1522.5781 | ESI-MS m/z: [M + H]+ = 1523.6 | Method 4; Purity: 97.78% |

### Example A16: Radiolabeling Protocols

General Protocol for Cold Labeling of Empty-Chelator Conjugates with Lu, La and Ga Metals

To a solution of peptide conjugated to an empty-chelator (1 equiv.) in 1M Na₂CO₃ aqueous buffer (pH=5-6) was added LuCl₃, LaCl₃, or GaCl₃ (5 equiv.). The resulting mixture was stirred at 40-60° C. for 1 h. After filtration, the crude product was purified by preparative HPLC to afford the corresponding cold metal labeled peptide.

### General Protocol for ¹⁷⁷Lu Radiolabeling of Empty-DOTA Conjugate

A 35 mCi aliquot of ¹⁷⁷LuCl₃ was added to the reaction vessel followed by addition of 10% ethanol in 0.4M NaOAc buffer (1.0 mL; pH=5.0) and the empty-DOTA conjugate (1.0 μg/μL, 32 μL). The resulting mixture was heated and mixed with a thermal mixer at 70° C. for 0.5 h. After 0.5 h, the reaction mixture was allowed to cool at room temperature for 3 min. Then the reaction mixture was diluted with 4.8 mL of a pre-mixed formulation buffer. To assess the radiochemical purity, 60 μCi of the ¹⁷⁷Lu labeled product was injected on a rad-HPLC. At the end of the synthesis (EOS), the radiochemical purity and specific activity was determined. HPLC column: Aeris 3.6 μm PEPTIDE XB-C18 100, LC Column, 150×4.6 mm (00F-4507-E0). Flow 1.0 mL/min. HPLC column Aeris 3.6 μm PEPTIDE XB-C18 100, LC Column, 150×4.6 mm (00F-4507-E0). Solvent A was H₂O/0.05% TFA and solvent B was CH₃CN/0.05% TFA. Elution was conducted at flow rate of 1.0 mL/min with gradient from 5 to 95% B over 16 min.

### General Protocol for ²²⁵Ac Radiolabeling of Empty-DOTA Conjugate

A ²²⁵Ac(NO₃) 3 powder was diluted with 0.5M HCl to achieve a concentration of 10 μCi/μL. 100 μCi aliquot of the 10 μCi/μL ²²⁵ Ac-chloride solution was added to the reaction vessel followed by addition of 10% ethanol in 0.4M NaOAc buffer (200 μL, pH=6.2) and 27.5 μg of the empty-DOTA conjugate (1.0 μg/μL, 27.5 μL). The resulting mixture was heated and mixed with a thermal mixer at 90° C. for 0.5 h. After 0.5 h, the reaction mixture was allowed to cool at room temperature for 3 min. The reaction mixture was diluted with 1.0 mL of a pre-mixed formulation buffer. The radiochemical purity was determined at 5 μCi/nmol by radio thin-layer chromatography (radio-TLC). TLC plate: Agilent Technologies Chromatography paper (Cat number A120B12); mobile phase: 50 mM DTPA, pH=5.56.

### General Protocol for ⁶⁴Cu Radiolabeling of Empty-NOTA Conjugate

⁶⁴CuCl₂ in 0.1M HCl was reconstituted with 0.4M NaOAc pH=6.2 to achieve a concentration of 0.2 mCi/μL. A 7.14 mCi aliquot of the ⁶⁴CuCl₂ solution was added to the reaction vessel followed by addition of 10% ethanol in 0.4M NaOAc buffer (200 μL; pH=6.2) and the empty-NOTA conjugate (1.0 μg/μL, 6.0 μL). The resulting mixture was heated and mixed with a thermal mixer at 50° C. for 0.5 h. After 0.5 h, the reaction mixture was allowed to cool at room temperature for 3 min. Then the reaction was diluted with 4.8 mL of a pre-mixed formulation buffer. To assess the radiochemical purity, 10 μCi of the product was injected on a rad-HPLC. At the end of the synthesis (EOS), the radiochemical purity and specific activity was determined. HPLC column: Aeris 3.6 μm PEPTIDE XB-C18 100, LC Column, 150×4.6 mm (00F-4507-E0). Solvent A was H₂O/0.05% TFA and solvent B was CH₃CN/0.05% TFA. Elution was conducted at flow rate of 1.0 mL/min with gradient from 5 to 95% B over 16 min.

### General Protocol for 68Ga Radiolabeling of Empty-DOTA Conjugate

A 10 mCi aliquot of 68GaCl in 0.1M HCl (1.1 mL) was added to the reaction vessel followed by addition of 1.0 M NaOAc, pH 5.5, 175 AL, and empty DOTA conjugate (0.75 μg/μL; 42 μL). The resulting mixture was heated and mixed with a thermal mixer at 95° C. for 10 min. After 10 min, the reaction mixture was allowed to cool at room temperature for 3 minutes. Then the reaction mixture was diluted with 4 mL of a pre-mixed formulation buffer. To assess the radiochemical purity, 10 μCi of the product was injected on a rad-HPLC. At the end of the synthesis (EOS), the radiochemical purity and specific activity was determined. HPLC column: Aeris 3.6 μm PEPTIDE XB-C18 100, LC Column, 150×4.6 mm (00F-4507-E0). Solvent A was H₂O/0.05% TFA and solvent B was CH₃CN/0.05% TFA. Elution was conducted at flow rate of 1.0 mL/min with gradient from 5 to 95% B over 16 min.

### Example B: Biological Examples

### Example B1: SSTR Binding Affinity Measured by Radioligand Binding Competition Assay

Macrocyclic peptides in this invention were tested for binding to SSTR protein by a radioligand binding competition assay. For radioligand binding assay, competition binding is performed in duplicate in the wells of a 96 well plate containing binding buffer (HEPES 25 mM pH 7.4, MgCl₂ 5 mM, CaCl₂ 1 mM, BSA 0.5%, Saponine 10 μg/ml)), membrane extracts (0.6 μg protein/well), radiotracer ([125I]-Tyr11-Somatostatin, Perkin Elmer NEX389) and test compound. Nonspecific binding is determined by co-incubation with 200-fold excess of cold competitor (SST28). The samples are incubated in a final volume of 0.1 ml for 60 min at room temperature and then filtered over UniFilter-96 GF/C (Revvity 6055690) filter plates. Filters are washed six times with 0.5 ml of ice-cold washing buffer (HEPES 25 mM pH 7.4, MgCl₂ 5 mM, CaCl₂ 1 mM) and 50 μl of Microscint 20 (Packard) are added in each well. The plates are incubated 15 min on an orbital shaker and then counted with a TopCount™ for 1 min/well. For IC50 to Ki determination, the Cheng Prusoff equation is used: Ki=IC50/(1+ [tracer]/Kd). [Tracer] is 0.1 nM as mentioned above.

Conjugate SSTR2 binding affinity is shown in Table 9.

TABLE 9

| Conjugate ID | SSTR2 Ki |
|---|---|
| RB-0002327 | A |
| RB-0002328 | B |
| RB-0002329 | B |
| RB-0002593 | C |
| RB-0002594 | A |
| RB-0002595 | A |
| RB-0002596 | A |
| RB-0002597 | B |
| RB-0002670 | A |
| RB-0002672 | B |
| RB-0002674 | B |
| RB-0002676 | A |
| RB-0002678 | C |
| RB-0002680 | B |
| RB-0002946 | D |
| RB-0002947 | C |
| RB-0003000 | A |
| RB-0003002 | A |
| RB-0003055 | A |
| RB-0003057 | A |
| RB-0003136 | B |
| RB-0003137 | A |
| RB-0003148 | A |
| RB-0003149 | A |
| RB-0003164 | B |
| RB-0003246 | B |
| RB-0003248 | C |
| RB-0003276 | C |
| RB-0003277 | B |
| RB-0003278 | B |
| RB-0003279 | C |
| RB-0003280 | A |
| RB-0003281 | C |
| RB-0003292 | A |
| RB-0003294 | B |
| RB-0003296 | B |
| RB-0003298 | C |
| RB-0003302 | C |
| RB-0003304 | C |
| RB-0003306 | B |
| RB-0003309 | A |
| RB-0003357 | A |
| RB-0003374 | A |
| RB-0003375 | B |
| RB-0003381 | A |
| RB-0003382 | A |
| RB-0003383 | A |
| RB-0003407 | B |
| RB-0003408 | B |
| RB-0003409 | A |
| RB-0003443 | D |
| RB-0003445 | C |
| RB-0003447 | C |
| RB-0003449 | D |
| RB-0003451 | C |
| RB-0003453 | A |
| RB-0003455 | A |
| RB-0003464 | A |
| RB-0003482 | A |
| RB-0003484 | A |
| RB-0003486 | A |
| RB-0003504 | B |
| RB-0004536 | A |
| RB-0004538 | C |
| RB-0004540 | A |
| RB-0004609 | A |
| RB-0004611 | A |
| RB-0004613 | A |
| RB-0004615 | A |
| RB-0004617 | B |
| RB-0004619 | A |
| RB-0004621 | A |
| RB-0004623 | A |
| RB-0004819 | A |
| RB-0004821 | B |
| RB-0004823 | A |
| RB-0004825 | A |
| RB-0004864 | B |
| RB-0004866 | D |
| RB-0004868 | C |
| RB-0005247 | C |
| RB-0005253 | C |
| RB-0005255 | B |
| RB-0005266 | A |
| RB-0005270 | A |
| RB-0005272 | A |
| RB-0005274 | A |
| RB-0005276 | B |
| RB-0005278 | B |
| RB-0005284 | A |
| RB-0005286 | D |
| RB-0005330 | A |
| RB-0005502 | A |
| RB-0005692 | B |
| RB-0005694 | B |
| RB-0005696 | D |
| RB-0005717 | D |
| RB-0005728 | C |
| RB-0005730 | A |
| RB-0005732 | E |
| RB-0005734 | B |
| RB-0005736 | C |
| RB-0005738 | A |
| RB-0005740 | A |
| RB-0005744 | A |
| RB-0005760 | A |
| RB-0005761 | B |
| RB-0005797 | A |
| RB-0005799 | A |
| RB-0005801 | B |
| RB-0005805 | A |
| RB-0005807 | A |
| RB-0005809 | B |
| RB-0005827 | D |
| RB-0005852 | E |
| RB-0005854 | D |
| RB-0005858 | B |
| RB-0005864 | B |
| RB-0005866 | B |
| RB-0005895 | C |
| RB-0005897 | A |
| RB-0005899 | A |
| RB-0005903 | A |
| RB-0005905 | A |
| RB-0005907 | C |
| RB-0005983 | C |
| RB-0005984 | B |
| RB-0005985 | A |
| RB-0005990 | A |
| RB-0005992 | C |
| RB-0005994 | A |
| RB-0006009 | B |
| RB-0006011 | B |
| RB-0006013 | B |
| RB-0006015 | B |
| RB-0006019 | A |
| RB-0006069 | C |
| RB-0006072 | A |
| RB-0006073 | C |
| RB-0006074 | C |
| RB-0006075 | B |
| RB-0006076 | FALSE |
| RB-0006095 | A |
| RB-0006128 | A |
| RB-0006139 | E |
| RB-0006158 | B |
| RB-0006160 | D |
| RB-0006162 | B |
| RB-0006166 | E |
| RB-0006170 | C |
| RB-0006174 | A |
| RB-0006176 | A |
| RB-0006189 | B |
| RB-0006209 | B |
| RB-0006213 | E |
| RB-0006215 | D |
| RB-0006217 | A |
| RB-0006237 | C |
| RB-0006239 | D |
| RB-0006285 | B |
| RB-0006286 | B |
| RB-0006317 | A |
| RB-0006319 | A |
| RB-0006385 | A |
| RB-0006387 | A |
| RB-0006389 | A |
| RB-0006391 | E |
| RB-0006393 | A |
| RB-0006395 | A |
| RB-0006397 | B |
| RB-0006400 | B |
| RB-0006403 | B |
| RB-0006406 | B |
| RB-0006414 | B |
| RB-0006417 | B |
| RB-0006420 | B |
| RB-0006449 | A |
| RB-0006452 | B |
| RB-0006455 | B |
| RB-0006458 | C |
| RB-0006461 | C |
| RB-0006464 | B |
| RB-0006470 | B |
| RB-0006476 | C |
| RB-0006488 | B |
| RB-0006553 | D |
| RB-0006558 | A |
| RB-0006579 | C |
| RB-0006591 | C |

wherein 0 < A < 1.0 nM
1.0 nM ≤ B < 3.33 nM
3.33 nM ≤ C < 10 nM
10 nM ≤ D < 33.3 nM
33.3 nM ≤ E < 100 nM

### Example B2: Peptide Conjugate Biodistribution Measured by Tissue PK

Macrocyclic peptides and conjugates described herein were tested for tissue distribution by measuring concentrations in tissues by MS using in vivo animal models. Peptide metal chelates were dosed to animals and tissues were collected at specific time points based off the peptide PK profile. The samples were then analyzed by MS and the results reported as % ID/g (% injected dose/gram). Radiopharmaceutical conjugates of the present disclosure have improved tissue distribution for radiopharmaceutical therapeutics.

Peptide metal chelate doses were suspended in phosphate-buffered saline (PBS) containing 0.1% Tween 80. An injection volume of 100 μL per mouse was used, and blood and tissue samples were collected, weighed, and stored frozen at-80° C. until analysis. Tissue samples were digested with concentrated nitric acid in a microwave digestion apparatus. The sample digest was diluted with internal standard and analyzed using inductively coupled plasma mass spectrometry (ICP-MS). ICP-MS is an elemental analysis technique used to measure elements by using argon plasma to convert the sample into ions that are then measured using the mass spectrometer. Calibration standards were used for quantification of element(s) of interest by constructing a multipoint standard curve covering the range of analyte concentrations anticipated in the samples (typical range is 500 ppt to 0.1 ppt). A single quadrupole instrument (iCAP RQ, Thermo Fisher) ICP-MS was coupled with a Teledyne CETAC autosampler and used for the sample analysis.

The % ID/g of conjugate in kidney tissue at the 2-hour time point (2 h) was quantified for various conjugates, and the results are shown in Table 10. Lower kidney tissue % ID/g values indicate lower distribution to the kidney after administration of the conjugate.

TABLE 10

| | | | Kidney Tissue |
|---|---|---|---|
| Conjugate ID | X3 | X4 | PK (% ID/g, 2 h) |
| RB-0003149 | D-Trp | Lys | 8.4 |
| RB-0005760 | (S-bMe) D-Trp | Cha4N | 2.7 |
| RB-0005799 | D-Trp | Lys(Me) | 2.3 |
| RB-0005801 | D-Trp | Lys(diMe) | 2 |
| RB-0005805 | D-Trp | PipzaA | 2.5 |
| RB-0005897 | D-Trp | (3-Azetidine)-hAla | 2.5 |
| RB-0006013 | D-Trp | Ser(3-azetidine) | 3 |
| RB-0006015 | D-Trp | Nva(NH-NH2) | 2.8 |
| RB-0006072 | (S-bMe) D-Trp | PipzaA | 2.7 |
| RB-0005502 | Trp | Lys | 9.4 |
| RB-0006176 | (S-bMe) D-Trp | PipzaA | 1.6 |

The present technology may be used in bio-related industries and the pharmaceutical industry.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations, or improvements therein may be subsequently made by those skilled in the art which are also intended to be encompassed by the following claims.

1. A conjugate or a pharmaceutically acceptable salt thereof, having a structure of Formula IV1: 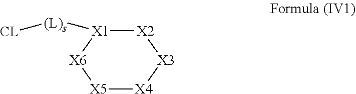
wherein:
X1 is any amino acid;
X2 is any amino acid;
X3 is a non-natural, aromatic amino acid;
X4 is a non-natural amino acid having a side chain comprising an amine;
X5 is any amino acid;
X6 is any amino acid;
L is a linker;
s is 0 or 1; and
CL is a metal chelator, wherein the metal chelator is 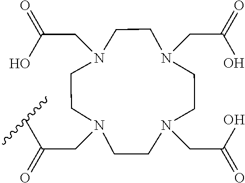 [SMILES: *C(=O)CN1CCN(CC(=O)O)CCN(CC(=O)O)CCN(CC(=O)O)CC1]
(DOTA).

2-5. (canceled)

6. The conjugate of claim 1, or a pharmaceutically acceptable salt thereof, wherein the conjugate further comprises a radionuclide bound to the metal chelator.

7-10. (canceled)

11. The conjugate of claim 1, or a pharmaceutically acceptable salt thereof, wherein:
X1 is Pro, Hyp, Cha4N, Chg4N, NMe-Cha4N, NMe-Chg4N, NMe-Dap, NMe-Dab, NMe-Orn, NMe-Lys, NMe-Azidolysine, NMe-hLys, 4-oxa NMe-Lys, NMe-Ala, NMe-Nle, NMe-propargyl glycine, NMe-propargyl alanine, NMe-Asp, NMe-Cys, NMe-Hcy, NMe-hHcy, NMe-Glu, NMe-hGlu, NMe-Amp, NMe-Hse, or NMe-Hse (Se);
X2 is Tyr, D-Tyr, 4Pal, 3Pal, 5F-Tyr, 3,5-diF-Tyr, Phe, (R-βMe) Phe, (S-βMe) Phe, 3,3-diPhe, D-Phg, L-DOPA, Aph(Hor), His, 2-(Aminocarbonyl)-Phe, 3-(Aminocarbonyl)-Phe, Ala, or Gly;
X3 is D-Trp, NMe-D-Trp, (S-βMe) D-Trp, (S-βMe) Trp, (R-βMe) D-Trp, (R-βMe) Trp, Aza-Trp, Aza-D-Trp, D-6F-Trp, or D-Aph(Cbm), each of which is further optionally substituted; or
Cba3N, Chg4N, Cha4N, Cha4NH₂, Ser(3-azetidine), PipzaA, 3-Azetidine-hAla, Pic4, NMe-Lys, Lys(Me), Lys(diMe), Lys(iPr), or 4-oxa-Lys;
X5 is Thr, Val, Ala, Pro, Alt, Cbg, Cpg, Cba, or Tme; or
X6 is Phe, NMe-Phe, (S-βMe) Phe, (R-βMe) Phe, His, Mpd, Ala, D-Ala, Gly, Pro, Ser, Ser(Ph), 3Pal, 4Pal, Cha, 3-(Aminocarbonyl)-Phe, F4COO, Phg, G(cPr), Asn, Tyr, meta-Tyr, or 3N-Tyr.

12-34. (canceled)

35. The conjugate of claim 1, or a pharmaceutically acceptable salt thereof, wherein X4 has a structure of: 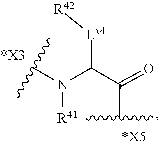
wherein,
R⁴¹ is hydrogen or C₁-C₅alkyl optionally substituted with one to three substituents independently selected from R^{f},
each R^{f} is independently halogen, —CN, —NO₂, —OR^{a}, —SR^{a} or —NR^{c}R^{d},
L^{X4} is a bond, —O—, —S—, —NR⁴³—, C₁-C₆alkylene, C₁-C₆heteroalkylene, C₃-C₆cycloalkyl, or 3- to 6-membered heterocycloalkyl, wherein the alkylene, heteroalkylene, cycloalkyl, or heterocycloalkyl is optionally substituted with one or more R^{X4a};
R⁴² is —NR⁴⁴R⁴⁵ or a heterocycloalkyl comprising one or more ring nitrogen atoms, wherein the heterocycloalkyl is optionally substituted with one or more R^{42a};
each R^{42a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, —O, —S, or ═N(R^{a}), wherein each of the alkyl, heteroalkyl, alkenyl, and alkynyl is optionally substituted with one or more R^{e};
R⁴³ is hydrogen or C₁-C₃alkyl;
R⁴⁴ and R⁴⁵ are each independently hydrogen, C₁-C₃alkyl, aryl, heteroaryl, —C₁-C₃alkylene-aryl, or —C₁-C₃alkylene-heteroaryl, wherein each of the alkyl, aryl, and heteroaryl are optionally substituted with one or more R^{42a},
each R^{X4a} is independently halogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR)(OR⁴), —P(═O)R^{c}R^{d}, ═O, ═S, or ═N(R^{a}), wherein each of the alkyl, heteroalkyl, alkenyl, and alkynyl is optionally substituted with one or more R^{e}; or
or two R^{X4a} groups attached to the same or different atoms are taken together to form a cycloalkyl or heterocycloalkyl ring, each of which is optionally substituted with one or more R^{e};
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e};
*X3 represents the point of attachment to X3; and
*X5 represents the point of attachment to X5.

36-47. (canceled)

48. The conjugate of claim 1, or a pharmaceutically acceptable salt thereof, wherein X4 is 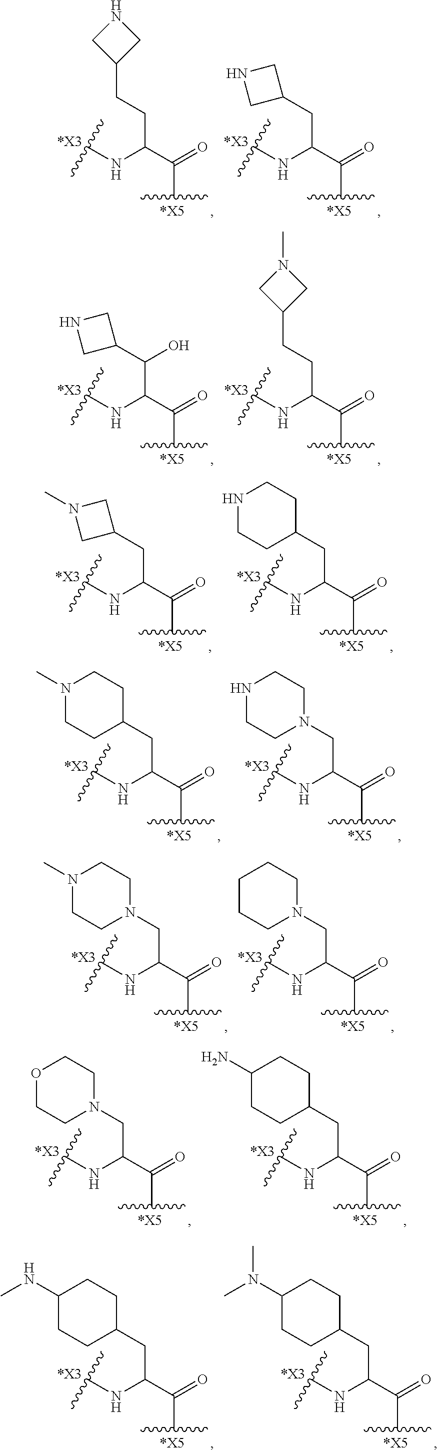 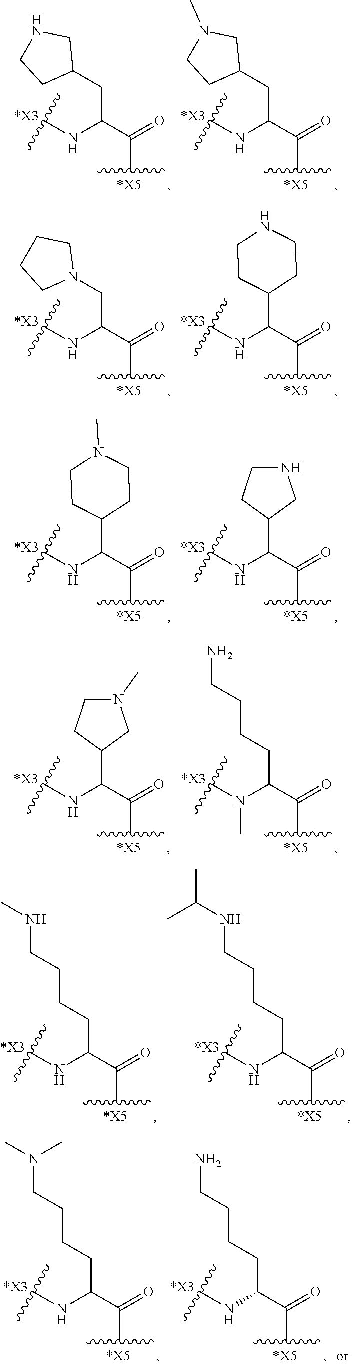 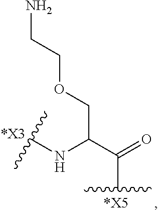 [SMILES: *NC(COCCN)C(*)=O]
wherein
*X3 represents the point of attachment to X3; and
*X5 represents the point of attachment to X5.

49-56. (canceled)

57. The conjugate of claim 1, or a pharmaceutically acceptable salt thereof, wherein
X1 is NMe-Hcy, NMe-Lys, or NMe-hLys;
X2 is Tyr;
X3 is D-Trp, (S-βMe) D-Trp, (S-βMe)-Trp, (R-βMe) D-Trp, or (R-βMe)-Trp;
X4 is PipzaA, 3-Azetidine-hAla, Lys(Me), Chg4N or Cha4N;
X5 is Thr or Alt; and
X6 is Phe.

58-83. (canceled)

84. The conjugate of claim 1, or a pharmaceutically acceptable salt thereof, wherein X3 has a structure of: 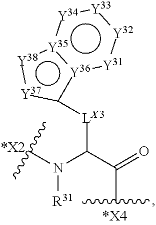
wherein:
R³¹ is hydrogen or C₁-C₅ alkyl optionally substituted with one to three substituents independently selected from R^{f};
each R^{f} is independently halogen, —CN, —NO₂, —OR^{a}, —SR^{a} or —NR^{c}R^{d};
L^{X3} is a bond, —O—, —S—, —NR³³—, C₁-C₃alkylene, or C₁-C₃heteroalkylene, wherein the alkylene or heteroalkylene is optionally substituted with one or more R^{X3a};
R³³ is hydrogen or C₁-C₃alkyl;
each R^{X3a} is independently halogen, —CN, —NO₂, —OR^{a}, —NR^{c}R^{d}, C₁-C₆alkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, cycloalkyl, heterocycloalkyl, aryl, and heteroaryl is independently optionally substituted with one or more R^{e};
or two R^{X3a} groups attached to the same or different atoms are taken together to form a cycloalkyl or heterocycloalkyl ring, each of which is optionally substituted with one or more R^{e}:
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}, or R^{e} and R^{d} are taken together with the atom to which they are attached to form a heterocycloalkyl optionally substituted with one or more R^{e};
*X2 represents the point of attachment to X2;
*X4 represents the point of attachment to X4;
Y³¹ is N, CH, or CR³²;
Y³² is N, CH, or CR³²;
Y³³ is N, CH, or CR³²;
Y³⁴ is N, CH, or CR³²;
Y³⁵ is N, or C;
Y³⁶ is N or C;
Y³⁷ is N, CH, or CR³²; and
Y³⁸ is S, N or NH;
provided that no more than two of Y³¹, Y³², Y³³, Y³⁴, Y³⁵, Y³⁶, and Y³⁷ are N.

85-103. (canceled)

104. The conjugate of claim 1, or a pharmaceutically acceptable salt thereof, wherein X3 is 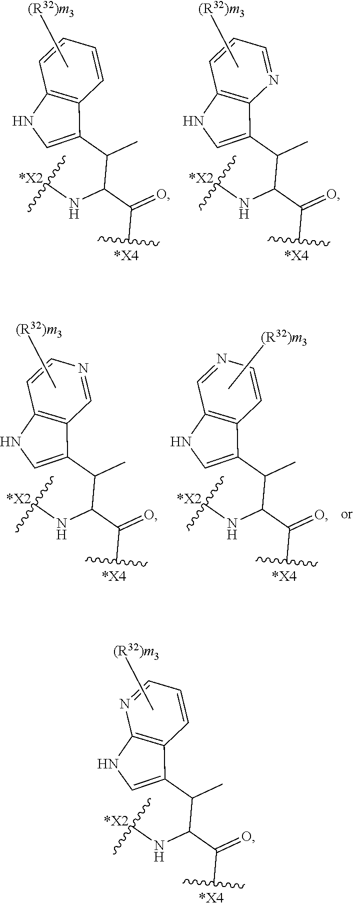
wherein each R³² is independently C₁-C₆alkyl, C₁-C₆haloalkyl, halogen, —CN, —OR^{a}, —SR^{a}, or —NR^{c}R^{d},
m3 is 0, 1, or 2;
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e};
each R^{e} is independently halogen, —CN, —OH, oxo, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆alkyl, C₁-C₆haloalkyl, C₁-C₆hydroxyalkyl, C₁-C₆aminoalkyl, or C₁-C₆heteroalkyl; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl), wherein each of the alkyl, alkylene, alkenyl, alkynyl, cycloalkyl, heterocycloalkyl, aryl, and heteroaryl is independently optionally substituted with one or more R^{e}, or R^{c} and R^{d} are taken together with the atom to which they are attached to form a heterocycloalkyl optionally substituted with one or more R^{e},
*X2 represents the point of attachment to X2; and
*X4 represents the point of attachment to X4.

105-153. (canceled)

154. The conjugate of claim 1, wherein the linker is a bond or

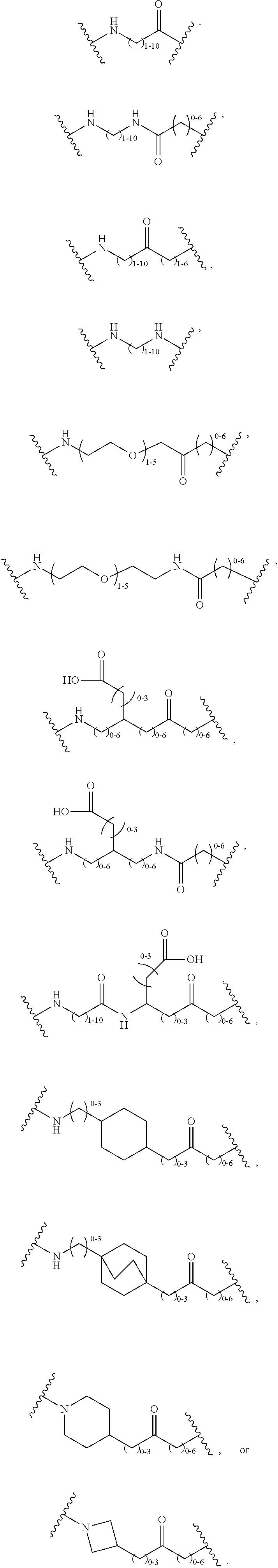

155. The conjugate of claim 154, wherein the linker is:
(i) a bond;

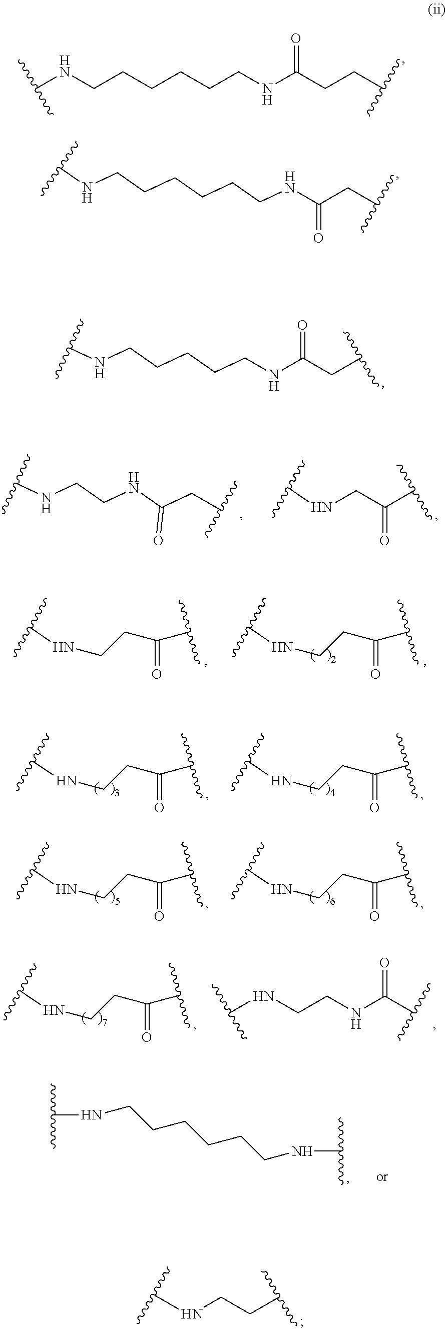

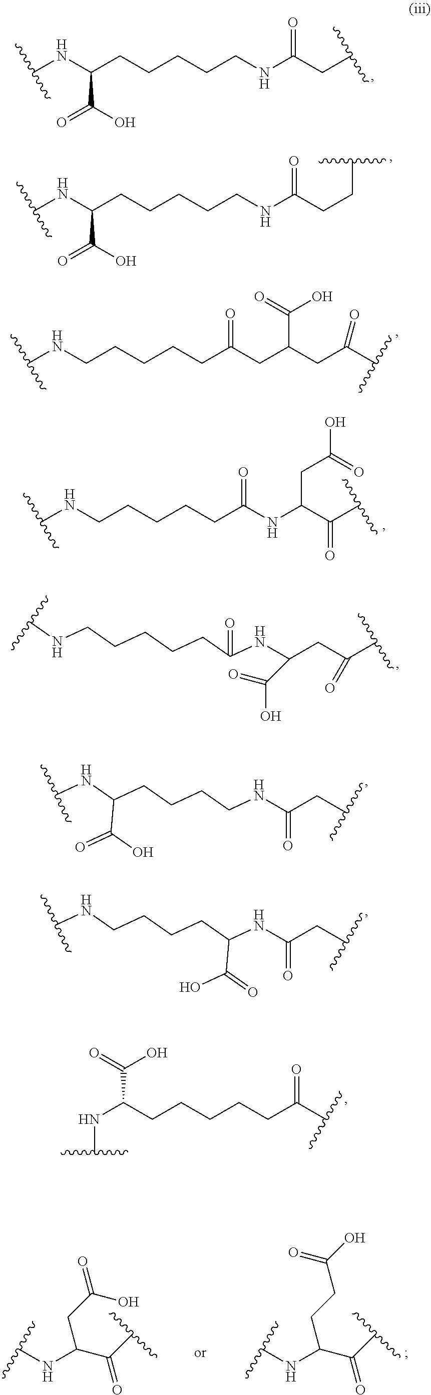

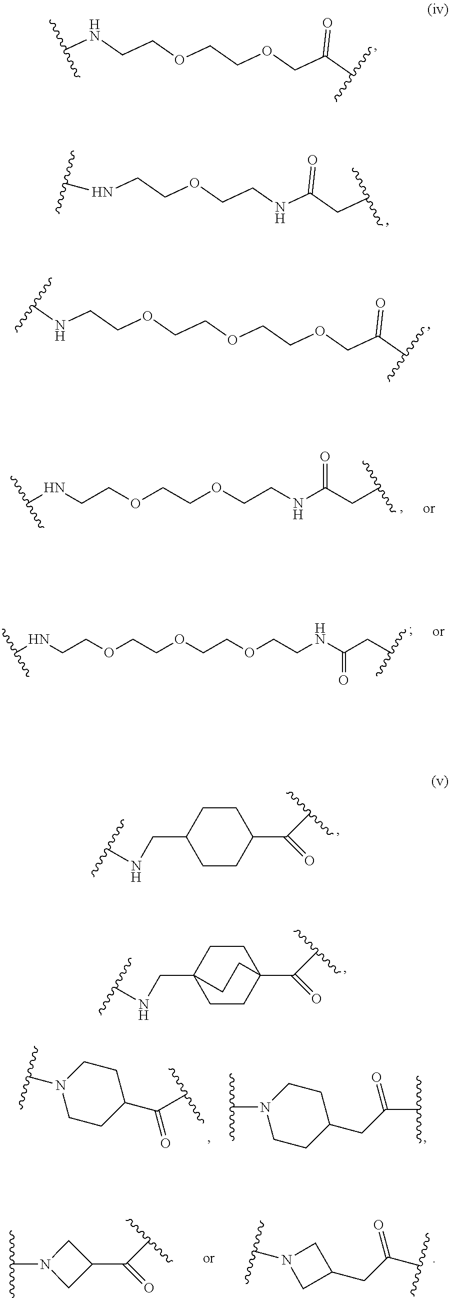

156. (canceled)

157. The conjugate of claim 1, of the following structural formula: 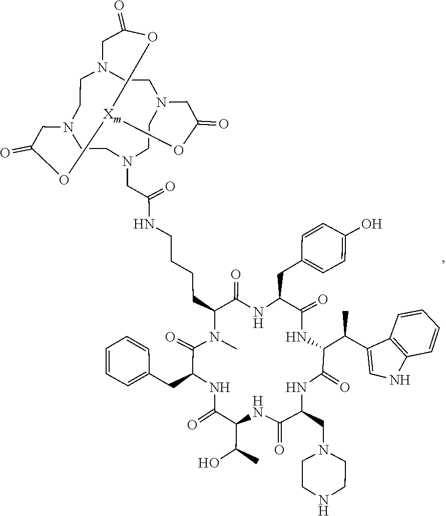
and pharmaceutically acceptable salts and stereoisomers thereof, wherein Xₘ is a radionuclide.

158-165. (canceled)

166. The conjugate of claim 157, or a pharmaceutically acceptable salt thereof, wherein the radionuclide is Ac-225.

167-173. (canceled)

174. A peptide having avidity for a somatostatin receptor, wherein the peptide comprises a structure of Formula (I) or a salt thereof,
X1-X2-X3-X4-X5-X6 Formula (I)
wherein,
X1 is any amino acid;
X2 is any amino acid;
X3 is a non-natural, aromatic amino acid;
X4 is a non-aromatic amino acid having a side chain comprising an amine;
X5 is any amino acid; and
X6 is any amino acid.

175. (canceled)

176. A method of treating a somatostatin receptor-positive (SSTR+) tumor in a subject in need thereof, comprising administering to the subject a conjugate of claim 6 or a pharmaceutically acceptable salt thereof.

177. A method of imaging or diagnosing a somatostatin receptor-positive (SSTR+) tumor in a subject, comprising administering to the subject a conjugate of claim 6 or a pharmaceutically acceptable salt thereof, wherein the radionuclide is selected from Ce-134, Cu-61, Cu-62, Cu-64, Ga-68, Ho-166, In-111, Tb-152, Tb-161, Tc-99, and Zr-89.

178. A pharmaceutical composition comprising a conjugate of claim 6 or a pharmaceutically acceptable salt thereof and a pharmaceutically acceptable excipient.

179. A method of treating a somatostatin receptor-positive (SSTR+) tumor in a subject in need thereof, comprising administering to the subject a conjugate of claim 157 or a pharmaceutically acceptable salt thereof.

180. A method of imaging or diagnosing a somatostatin receptor-positive (SSTR+) tumor in a subject, comprising administering to the subject a conjugate of claim 157 or a pharmaceutically acceptable salt thereof, wherein the radionuclide is selected from Ce-134, Cu-61, Cu-62, Cu-64, Ga-68, Ho-166, In-111, Tb-152, Tb-161, Tc-99, and Zr-89.

181. A pharmaceutical composition comprising a conjugate of claim 157 or a pharmaceutically acceptable salt thereof and a pharmaceutically acceptable excipient.

182. The conjugate of claim 157, of the following structural formula: 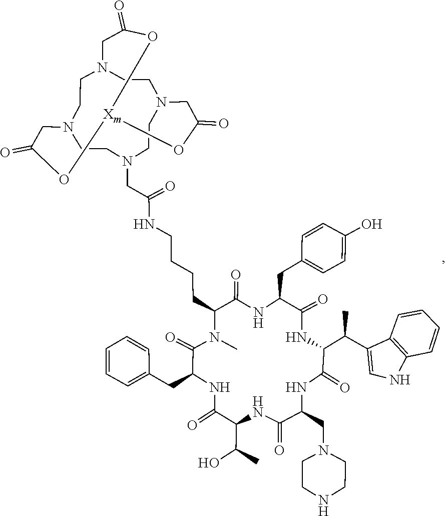
or a pharmaceutically acceptable salt thereof,
wherein Xₘ is Ac-225.

183. The conjugate of claim 157, of the following structural formula: 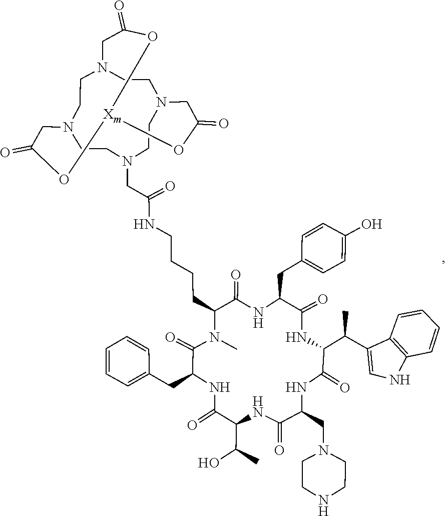
wherein Xₘ is Ac-225.

184. A conjugate or a pharmaceutically acceptable salt thereof, having a structure of Formula IV1: 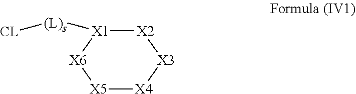
wherein:
X1 is any amino acid;
X2 is any amino acid;
X3 is a non-natural, aromatic amino acid;
X4 is a non-natural amino acid having a side chain comprising an amine;
X5 is any amino acid;
X6 is any amino acid;
L is a linker;
s is 0 or 1; and
CL is a metal chelator, wherein the metal chelator is

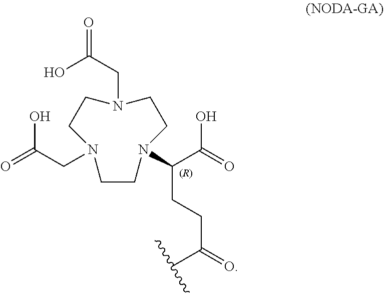

185. A method of treating a somatostatin receptor-positive (SSTR+) tumor in a subject in need thereof, comprising administering to the subject a conjugate of claim 184 or a pharmaceutically acceptable salt thereof.

186. A method of imaging or diagnosing a somatostatin receptor-positive (SSTR+) tumor in a subject, comprising administering to the subject a conjugate of claim 184 or a pharmaceutically acceptable salt thereof, wherein the radionuclide is selected from Ce-134, Cu-61, Cu-62, Cu-64, Ga-68, Ho-166, In-111, Tb-152, Tb-161, Tc-99m, and Zr-89.

187. A pharmaceutical composition comprising a conjugate of claim 184 or a pharmaceutically acceptable salt thereof and a pharmaceutically acceptable excipient.


SEQUENCE LISTING (335 sequences)

SEQ ID NO: 1 - protein, 6 aa, synthetic construct
  SITE 1: note = methyl-alanine
  SITE 3: note = D-tryptophan
  SITE 1..6: note = Cyclic
AYWKVF

SEQ ID NO: 2 - protein, 6 aa, synthetic construct
  SITE 1: note = methyl-alanine
  SITE 3: note = (R)-2-amino-3-(4-ureidophenyl)propanoic acid
  SITE 1..6: note = Cyclic
AYXKVF

SEQ ID NO: 3 - protein, 6 aa, synthetic construct
  SITE 1: note = methyl-lysine
  SITE 3: note = D-tryptophan
  SITE 1..6: note = Cyclic
KYWKVF

SEQ ID NO: 4 - protein, 6 aa, synthetic construct
  SITE 1: note = methyl-D-lysine
  SITE 3: note = D-tryptophan
  SITE 1..6: note = Cyclic
KYWKVF

SEQ ID NO: 5 - protein, 6 aa, synthetic construct
  SITE 1: note = methyl-lysine
  SITE 3: note = D-tryptophan
  SITE 1..6: note = Cyclic
KYWKTF

SEQ ID NO: 6 - protein, 6 aa, synthetic construct
  SITE 1: note = methyl-alanine
  SITE 2: note = 2-amino-3-(3,4-dihydroxyphenyl)propanoic acid
  SITE 3: note = D-tryptophan
  SITE 1..6: note = Cyclic
AXWKVF

SEQ ID NO: 7 - protein, 6 aa, synthetic construct
  SITE 1: note = methyl-alanine
  SITE 2: note = 2-amino-3,3-diphenylpropanoic acid
  SITE 3: note = D-tryptophan
  SITE 1..6: note = Cyclic
AFWKVF

SEQ ID NO: 8 - protein, 6 aa, synthetic construct
  SITE 1: note = methyl-alanine
  SITE 2: note = 2-amino-3-(4-(2,6-dioxohexahydropyrimidine-4-carbox amido)phenyl)propanoic acid
  SITE 3: note = D-tryptophan
  SITE 1..6: note = Cyclic
AXWKVF

SEQ ID NO: 9 - protein, 6 aa, synthetic construct
  SITE 1: note = 5-amino-2-(methylamino)pentanoic acid
  SITE 3: note = D-tryptophan
  SITE 1..6: note = Cyclic
XYWKVF

SEQ ID NO: 10 - protein, 6 aa, synthetic construct
  SITE 1: note = methyl-cysteine
  SITE 3: note = D-tryptophan
  SITE 1..6: note = Cyclic
CYWKVF

SEQ ID NO: 11 - protein, 6 aa, synthetic construct
  SITE 1: note = methyl-homocysteine
  SITE 3: note = D-tryptophan
  SITE 1..6: note = Cyclic
XYWKVF

SEQ ID NO: 12 - protein, 6 aa, synthetic construct
  SITE 1: note = methyl-glutamate
  SITE 3: note = D-tryptophan
  SITE 1..6: note = Cyclic
EYWKVF

SEQ ID NO: 13 - protein, 6 aa, synthetic construct
  SITE 1: note = 2-(methylamino)hexanedioic acid
  SITE 3: note = D-tryptophan
  SITE 1..6: note = Cyclic
EYWKVF

SEQ ID NO: 14 - protein, 6 aa, synthetic construct
  SITE 1: note = methyl-cysteine
  SITE 2: note = 2-amino-3-(pyridin-3-yl)propanoic acid
  SITE 3: note = D-tryptophan
  SITE 1..6: note = Cyclic
CFWKTF

SEQ ID NO: 15 - protein, 6 aa, synthetic construct
  SITE 1: note = methyl-cysteine
  SITE 2: note = 2-amino-3-(pyridin-4-yl)propanoic acid
  SITE 3: note = D-tryptophan
  SITE 1..6: note = Cyclic
CFWKTF

SEQ ID NO: 16 - protein, 6 aa, synthetic construct
  SITE 1: note = methyl-homocysteine
  SITE 2: note = 2-amino-3-(pyridin-3-yl)propanoic acid
  SITE 3: note = D-tryptophan
  SITE 1..6: note = Cyclic
XFWKTF

SEQ ID NO: 17 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 1..6: note = Cyclic
XFWKTF

SEQ ID NO: 18 - protein, 6 aa, synthetic construct
  SITE 1: note = 2-(methylamino)heptanedioic acid
  SITE 3: note = D-tryptophan
  SITE 1..6: note = Cyclic
XYWKTF

SEQ ID NO: 19 - protein, 6 aa, synthetic construct
  SITE 1: note = 7-amino-2-(methylamino)heptanoic acid
  SITE 3: note = D-tryptophan
  SITE 1..6: note = Cyclic
KYWKTF

SEQ ID NO: 20 - protein, 6 aa, synthetic construct
  SITE 1: note = methyl-homocysteine
  SITE 2: note = (2S,3S)-2-amino-3-phenylbutanoic acid
  SITE 3: note = D-tryptophan
  SITE 1..6: note = Cyclic
XFWKTF

SEQ ID NO: 21 - protein, 6 aa, synthetic construct
  SITE 1: note = methyl-homocysteine
  SITE 2: note = (2S,3R)-2-amino-3-phenylbutanoic acid
  SITE 3: note = D-tryptophan
  SITE 1..6: note = Cyclic
XFWKTF

SEQ ID NO: 22 - protein, 6 aa, synthetic construct
  SITE 1: note = methyl-homocysteine
  SITE 3: note = (2S,3S)-2-amino-3-(1H-indol-3-yl)butanoic acid
  SITE 1..6: note = Cyclic
XYWKTF

SEQ ID NO: 23 - protein, 6 aa, synthetic construct
  SITE 1: note = methyl-homocysteine
  SITE 3: note = D-tryptophan
  SITE 1..6: note = Cyclic
XYWKTF

SEQ ID NO: 24 - protein, 6 aa, synthetic construct
  SITE 1: note = methyl-cysteine
  SITE 3: note = D-tryptophan
  SITE 1..6: note = Cyclic
CYWKTF

SEQ ID NO: 25 - protein, 6 aa, synthetic construct
  SITE 1: note = methyl-homocysteine
  SITE 3: note = D-tryptophan
  SITE 6: note = 2-amino-3-(pyridin-3-yl)propanoic acid
  SITE 1..6: note = Cyclic
XYWKTF

SEQ ID NO: 26 - protein, 6 aa, synthetic construct
  SITE 1: note = methyl-homocysteine
  SITE 3: note = D-tryptophan
  SITE 6: note = 2-amino-3-(pyridin-4-yl)propanoic acid
  SITE 1..6: note = Cyclic
XYWKTF

SEQ ID NO: 27 - protein, 6 aa, synthetic construct
  SITE 1: note = methyl-homocysteine
  SITE 3: note = D-tryptophan
  SITE 6: note = (2S,3S)-2-amino-3-phenylbutanoic acid
  SITE 1..6: note = Cyclic
XYWKTF

SEQ ID NO: 28 - protein, 6 aa, synthetic construct
  SITE 1: note = methyl-homocysteine
  SITE 3: note = D-tryptophan
  SITE 6: note = 2-amino-3-(6-hydroxypyridin-3-yl)propanoic acid
  SITE 1..6: note = Cyclic
XYWKTY

SEQ ID NO: 29 - protein, 6 aa, synthetic construct
  SITE 1: note = methyl-homocysteine
  SITE 2: note = 2-amino-3-(3-fluoro-4-hydroxyphenyl)propanoic acid
  SITE 3: note = D-tryptophan
  SITE 1..6: note = Cyclic
XYWKTF

SEQ ID NO: 30 - protein, 6 aa, synthetic construct
  SITE 1: note = methyl-homocysteine
  SITE 3: note = D-tryptophan
  SITE 6: note = (2S,3R)-2-amino-3-phenylbutanoic acid
  SITE 1..6: note = Cyclic
XYWKTF

SEQ ID NO: 31 - protein, 6 aa, synthetic construct
  SITE 1: note = methyl-homocysteine
  SITE 2: note = 2-amino-3-(3-carbamoylphenyl)propanoic acid
  SITE 3: note = D-tryptophan
  SITE 1..6: note = Cyclic
XFWKTF

SEQ ID NO: 32 - protein, 6 aa, synthetic construct
  SITE 1: note = methyl-homocysteine
  SITE 2: note = 2-amino-3-(2-carbamoylphenyl)propanoic acid
  SITE 3: note = D-tryptophan
  SITE 1..6: note = Cyclic
XFWKTF

SEQ ID NO: 33 - protein, 6 aa, synthetic construct
  SITE 1: note = methyl-homocysteine
  SITE 3: note = D-tryptophan
  SITE 6: note = 2-amino-3-(3-carbamoylphenyl)propanoic acid
  SITE 1..6: note = Cyclic
XYWKTF

SEQ ID NO: 34 - protein, 6 aa, synthetic construct
  SITE 1: note = methyl-homocysteine
  SITE 3: note = (R)-2-amino-3-(6-fluoro-1H-indol-3-yl)propanoic acid
  SITE 1..6: note = Cyclic
XYWKTF

SEQ ID NO: 35 - protein, 6 aa, synthetic construct
  SITE 1: note = methyl-homocysteine
  SITE 3: note = D-tryptophan
  SITE 5: note = allothreonine
  SITE 1..6: note = Cyclic
XYWKTF

SEQ ID NO: 36 - protein, 6 aa, synthetic construct
  SITE 1: note = methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 1..6: note = Cyclic
XAWKTF

SEQ ID NO: 37 - protein, 6 aa, synthetic construct
  SITE 1: note = methyl-homocysteine
  SITE 2: note = 2-amino-3-(pyridin-3-yl)propanoic acid
  SITE 3: note = (2R,3S)-2-amino-3-(1H-indol-3-yl)butanoic acid
  SITE 1..6: note = Cyclic
XAWKTF

SEQ ID NO: 38 - protein, 6 aa, synthetic construct
  SITE 1: note = methyl-homocysteine
  SITE 2: note = (2S,3R)-2-amino-3-phenylbutanoic acid
  SITE 3: note = (2R,3S)-2-amino-3-(1H-indol-3-yl)butanoic acid
  SITE 1..6: note = Cyclic
XFWKTF

SEQ ID NO: 39 - protein, 6 aa, synthetic construct
  SITE 1: note = methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 6: note = 2-amino-3-(pyridin-3-yl)propanoic acid
  SITE 1..6: note = Cyclic
XAWKTF

SEQ ID NO: 40 - protein, 6 aa, synthetic construct
  SITE 1: note = methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 6: note = (2S,3R)-2-amino-3-phenylbutanoic acid
  SITE 1..6: note = Cyclic
XAWKTF

SEQ ID NO: 41 - protein, 6 aa, synthetic construct
  SITE 1: note = methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 6: note = (2S,3S)-2-amino-3-phenylbutanoic acid
  SITE 1..6: note = Cyclic
XAWKTF

SEQ ID NO: 42 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 6: note = 2-amino-3-(pyridin-4-yl)propanoic acid
  SITE 1..6: note = Cyclic
XAWKTF

SEQ ID NO: 43 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 6: note = 2-amino-3-(pyridin-3-yl)propanoic acid
  SITE 1..6: note = Cyclic
XAWKTF

SEQ ID NO: 44 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 6: note = (2S,3S)-2-amino-3-phenylbutanoic acid
  SITE 1..6: note = Cyclic
XAWKTF

SEQ ID NO: 45 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 6: note = (2S,3R)-2-amino-3-phenylbutanoic acid
  SITE 1..6: note = Cyclic
XAWKTF

SEQ ID NO: 46 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 6: note = 2-amino-3-(6-hydroxypyridin-3-yl)propanoic acid
  SITE 1..6: note = Cyclic
XAWKTY

SEQ ID NO: 47 - protein, 6 aa, synthetic construct
  SITE 1: note = methyl-homocysteine
  SITE 3: note = D-tryptophan
  SITE 4: note = methyl-lysine
  SITE 1..6: note = Cyclic
XYWKTF

SEQ ID NO: 48 - protein, 6 aa, synthetic construct
  SITE 1: note = methyl-homocysteine
  SITE 3: note = methyl-D-tryptophan
  SITE 4: note = methyl-lysine
  SITE 1..6: note = Cyclic
XYWKTF

SEQ ID NO: 49 - protein, 6 aa, synthetic construct
  SITE 1: note = 5-mercapto-2-(methylamino)pentanoic acid
  SITE 3: note = D-tryptophan
  SITE 1..6: note = Cyclic
XYWKTF

SEQ ID NO: 50 - protein, 6 aa, synthetic construct
  SITE 1: note = methyl-homocysteine
  SITE 2: note = 2-amino-3-(pyridin-4-yl)propanoic acid
  SITE 3: note = D-tryptophan
  SITE 1..6: note = Cyclic
XAWKTF

SEQ ID NO: 51 - protein, 6 aa, synthetic construct
  SITE 1: note = methyl-homocysteine
  SITE 3: note = methyl-D-tryptophan
  SITE 4: note = methyl-lysine
  SITE 6: note = methyl-phenylalanine
  SITE 1..6: note = Cyclic
XYWKTF

SEQ ID NO: 52 - protein, 6 aa, synthetic construct
  SITE 1: note = methyl-homocysteine
  SITE 3: note = D-tryptophan
  SITE 5: note = 2-amino-2-cyclobutylacetic acid
  SITE 1..6: note = Cyclic
XYWKXF

SEQ ID NO: 53 - protein, 6 aa, synthetic construct
  SITE 1: note = methyl-homocysteine
  SITE 3: note = (2R,3S)-2-amino-3-(1H-indol-3-yl)butanoic acid
  SITE 1..6: note = Cyclic
XYWKVF

SEQ ID NO: 54 - protein, 6 aa, synthetic construct
  SITE 1: note = methyl-homocysteine
  SITE 3: note = methyl-D-tryptophan
  SITE 4: note = methyl-lysine
  SITE 1..6: note = Cyclic
XYWKVF

SEQ ID NO: 55 - protein, 6 aa, synthetic construct
  SITE 1: note = methyl-homocysteine
  SITE 2: note = 2-amino-3,3-diphenylpropanoic acid
  SITE 3: note = D-tryptophan
  SITE 1..6: note = Cyclic
XFWKVF

SEQ ID NO: 56 - protein, 6 aa, synthetic construct
  SITE 1: note = methyl-homocysteine
  SITE 3: note = D-tryptophan
  SITE 6: note = 2-amino-3-(methyl(phenyl)amino)propanoic acid
  SITE 1..6: note = Cyclic
XYWKVX

SEQ ID NO: 57 - protein, 6 aa, synthetic construct
  SITE 1: note = methyl-homocysteine
  SITE 3: note = D-tryptophan
  SITE 6: note = 2-amino-3-cyclohexylpropanoic acid
  SITE 1..6: note = Cyclic
XYWKVX

SEQ ID NO: 58 - protein, 6 aa, synthetic construct
  SITE 1: note = methyl-homocysteine
  SITE 3: note = D-tryptophan
  SITE 5: note = O-methyl-threonine
  SITE 1..6: note = Cyclic
XYWKXF

SEQ ID NO: 59 - protein, 6 aa, synthetic construct
  SITE 1: note = methyl-homocysteine
  SITE 3: note = D-tryptophan
  SITE 5: note = 2-amino-3-cyclobutylpropanoic acid
  SITE 1..6: note = Cyclic
XYWKXF

SEQ ID NO: 60 - protein, 6 aa, synthetic construct
  SITE 1: note = methyl-homocysteine
  SITE 3: note = D-tryptophan
  SITE 5: note = 2-amino-2-cyclopentylacetic acid
  SITE 1..6: note = Cyclic
XYWKXF

SEQ ID NO: 61 - protein, 6 aa, synthetic construct
  SITE 1: note = methyl-homocysteine
  SITE 3: note = D-tryptophan
  SITE 1..6: note = Cyclic
XYWKTG

SEQ ID NO: 62 - protein, 6 aa, synthetic construct
  SITE 1: note = methyl-homocysteine
  SITE 3: note = D-tryptophan
  SITE 6: note = 2-amino-2-phenylacetic acid
  SITE 1..6: note = Cyclic
XYWKTX

SEQ ID NO: 63 - protein, 6 aa, synthetic construct
  SITE 1: note = methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 1..6: note = Cyclic
XAWKVF

SEQ ID NO: 64 - protein, 6 aa, synthetic construct
  SITE 1: note = methyl-homocysteine
  SITE 3: note = D-tryptophan
  SITE 4: note = 2-amino-3-(piperidin-4-yl)propanoic acid
  SITE 1..6: note = Cyclic
XYWXTF

SEQ ID NO: 65 - protein, 6 aa, synthetic construct
  SITE 1: note = methyl-homocysteine
  SITE 3: note = D-tryptophan
  SITE 4: note = 2-amino-3-(azetidin-3-yl)propanoic acid
  SITE 1..6: note = Cyclic
XYWXTF

SEQ ID NO: 66 - protein, 6 aa, synthetic construct
  SITE 1: note = methyl-homocysteine
  SITE 3: note = D-tryptophan
  SITE 4: note = 2-amino-2-(piperidin-4-yl)acetic acid
  SITE 1..6: note = Cyclic
XYWXTF

SEQ ID NO: 67 - protein, 6 aa, synthetic construct
  SITE 1: note = methyl-homocysteine
  SITE 3: note = D-tryptophan
  SITE 6: note = 2-amino-2-cyclopropylacetic acid
  SITE 1..6: note = Cyclic
XYWKTX

SEQ ID NO: 68 - protein, 6 aa, synthetic construct
  SITE 1: note = methyl-homocysteine
  SITE 3: note = D-tryptophan
  SITE 1..6: note = Cyclic
XYWKTN

SEQ ID NO: 69 - protein, 6 aa, synthetic construct
  SITE 1: note = methyl-homocysteine
  SITE 3: note = D-tryptophan
  SITE 1..6: note = Cyclic
XYWKTH

SEQ ID NO: 70 - protein, 6 aa, synthetic construct
  SITE 1: note = methyl-homocysteine
  SITE 3: note = D-tryptophan
  SITE 1..6: note = Cyclic
XYWKTA

SEQ ID NO: 71 - protein, 6 aa, synthetic construct
  SITE 1: note = methyl-homocysteine
  SITE 3: note = D-tryptophan
  SITE 1..6: note = Cyclic
XYWKTS

SEQ ID NO: 72 - protein, 6 aa, synthetic construct
  SITE 1: note = methyl-homocysteine
  SITE 3: note = methyl-D-tryptophan
  SITE 1..6: note = Cyclic
XYWKTF

SEQ ID NO: 73 - protein, 6 aa, synthetic construct
  SITE 1: note = methyl-homocysteine
  SITE 1..6: note = Cyclic
XYWKTF

SEQ ID NO: 74 - protein, 6 aa, synthetic construct
  SITE 1: note = methyl-homocysteine
  SITE 3: note = D-tryptophan
  SITE 6: note = methyl-phenylalanine
  SITE 1..6: note = Cyclic
XYWKTF

SEQ ID NO: 75 - protein, 6 aa, synthetic construct
  SITE 1: note = methyl-homocysteine
  SITE 3: note = D-tryptophan
  SITE 1..6: note = Cyclic
XGWKTF

SEQ ID NO: 76 - protein, 6 aa, synthetic construct
  SITE 1: note = methyl-homocysteine
  SITE 3: note = D-tryptophan
  SITE 1..6: note = Cyclic
XAWKTF

SEQ ID NO: 77 - protein, 6 aa, synthetic construct
  SITE 1: note = 4-amino-2-(methylamino)butanoic acid
  SITE 3: note = D-tryptophan
  SITE 1..6: note = Cyclic
XYWKTF

SEQ ID NO: 78 - protein, 6 aa, synthetic construct
  SITE 1: note = 3-amino-2-(methylamino)propanoic acid
  SITE 3: note = D-tryptophan
  SITE 1..6: note = Cyclic
XYWKTF

SEQ ID NO: 79 - protein, 6 aa, synthetic construct
  SITE 1: note = methyl-homocysteine
  SITE 3: note = D-tryptophan
  SITE 6: note = O-phenyl-serine
  SITE 1..6: note = Cyclic
XYWKTS

SEQ ID NO: 80 - protein, 6 aa, synthetic construct
  SITE 1: note = methyl-homocysteine
  SITE 2: note = 2-amino-3-(3,5-difluoro-4-hydroxyphenyl)propanoic acid
  SITE 3: note = D-tryptophan
  SITE 1..6: note = Cyclic
XYWKTF

SEQ ID NO: 81 - protein, 6 aa, synthetic construct
  SITE 1: note = methyl-homocysteine
  SITE 3: note = D-tryptophan
  SITE 1..6: note = Cyclic
XYWKAF

SEQ ID NO: 82 - protein, 6 aa, synthetic construct
  SITE 1: note = methyl-homocysteine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 83 - protein, 6 aa, synthetic construct
  SITE 1: note = methyl-homocysteine
  SITE 4: note = 2-amino-3-(piperidin-4-yl)propanoic acid
  SITE 1..6: note = Cyclic
XYWXTF

SEQ ID NO: 84 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 85 - protein, 6 aa, synthetic construct
  SITE 1: note = methyl-homocysteine
  SITE 3: note = D-tryptophan
  SITE 4: note = N6-methyl-lysine
  SITE 1..6: note = Cyclic
XYWKTF

SEQ ID NO: 86 - protein, 6 aa, synthetic construct
  SITE 1: note = methyl-homocysteine
  SITE 3: note = D-tryptophan
  SITE 4: note = N6,N6-dimethyl-lysine
  SITE 1..6: note = Cyclic
XYWKTF

SEQ ID NO: 87 - protein, 6 aa, synthetic construct
  SITE 1: note = methyl-homocysteine
  SITE 3: note = D-tryptophan
  SITE 4: note = 2-amino-3-(piperazin-1-yl)propanoic acid
  SITE 1..6: note = Cyclic
XYWXTF

SEQ ID NO: 88 - protein, 6 aa, synthetic construct
  SITE 1: note = methyl-homocysteine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTA

SEQ ID NO: 89 - protein, 6 aa, synthetic construct
  SITE 1: note = methyl-homocysteine
  SITE 2: note = 2-amino-3,3-diphenylpropanoic acid
  SITE 3: note = D-tryptophan
  SITE 4: note = 2-amino-3-(piperidin-4-yl)propanoic acid
  SITE 1..6: note = Cyclic
XFWXTA

SEQ ID NO: 90 - protein, 6 aa, synthetic construct
  SITE 1: note = methyl-homocysteine
  SITE 3: note = D-tryptophan
  SITE 4: note = D-Lysine
  SITE 1..6: note = Cyclic
XYWKTF

SEQ ID NO: 91 - protein, 6 aa, synthetic construct
  SITE 1: note = 2-amino-2-(piperidin-4-yl)acetic acid
  SITE 3: note = D-tryptophan
  SITE 1..6: note = Cyclic
XYWKTF

SEQ ID NO: 92 - protein, 6 aa, synthetic construct
  SITE 1: note = 2-amino-3-(piperidin-4-yl)propanoic acid
  SITE 3: note = D-tryptophan
  SITE 1..6: note = Cyclic
XYWKTF

SEQ ID NO: 93 - protein, 6 aa, synthetic construct
  SITE 1: note = methyl-homocysteine
  SITE 3: note = 4-aza-Trp
  SITE 1..6: note = Cyclic
XYWKTF

SEQ ID NO: 94 - protein, 6 aa, synthetic construct
  SITE 1: note = methyl-homocysteine
  SITE 3: note = D-tryptophan
  SITE 1..6: note = Cyclic
XYWKTP

SEQ ID NO: 95 - protein, 6 aa, synthetic construct
  SITE 1: note = methyl-D-homocysteine
  SITE 3: note = D-tryptophan
  SITE 1..6: note = Cyclic
XYWKTF

SEQ ID NO: 96 - protein, 6 aa, synthetic construct
  SITE 1: note = methyl-L-aspartate
  SITE 3: note = D-tryptophan
  SITE 1..6: note = Cyclic
DYWKTF

SEQ ID NO: 97 - protein, 6 aa, synthetic construct
  SITE 1: note = methyl-homocysteine
  SITE 3: note = D-tryptophan
  SITE 4: note = 2-amino-4-(azetidin-3-yl)butanoic acid
  SITE 1..6: note = Cyclic
XYWATF

SEQ ID NO: 98 - protein, 6 aa, synthetic construct
  SITE 1: note = methyl-homocysteine
  SITE 3: note = D-tryptophan
  SITE 4: note = O-(2-aminoethyl)-serine
  SITE 1..6: note = Cyclic
XYWXTF

SEQ ID NO: 99 - protein, 6 aa, synthetic construct
  SITE 1: note = methyl-homocysteine
  SITE 3: note = 2-amino-3-(1H-pyrrolo[3,2-c]pyridin-3-yl)propanoic acid
  SITE 1..6: note = Cyclic
XYWKTF

SEQ ID NO: 100 - protein, 6 aa, synthetic construct
  SITE 1: note = methyl-homocysteine
  SITE 3: note = 2-amino-3-(1H-pyrrolo[2,3-c]pyridin-3-yl)propanoic acid
  SITE 1..6: note = Cyclic
XYWKTF

SEQ ID NO: 101 - protein, 6 aa, synthetic construct
  SITE 1: note = methyl-homocysteine
  SITE 3: note = 2-amino-3-(1H-pyrrolo[2,3-b]pyridin-3-yl)propanoic acid
  SITE 1..6: note = Cyclic
XYWKTF

SEQ ID NO: 102 - protein, 6 aa, synthetic construct
  SITE 1: note = methyl-lysine
  SITE 3: note = D-tryptophan
  SITE 4: note = 2-amino-3-(piperidin-4-yl)propanoic acid
  SITE 1..6: note = Cyclic
KYWXTY

SEQ ID NO: 103 - protein, 6 aa, synthetic construct
  SITE 1: note = methyl-lysine
  SITE 3: note = D-tryptophan
  SITE 4: note = 2-amino-3-(piperidin-4-yl)propanoic acid
  SITE 6: note = 2-amino-3-(3-hydroxyphenyl)propanoic acid
  SITE 1..6: note = Cyclic
KYWXTY

SEQ ID NO: 104 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 6: note = 2-amino-3-(3-hydroxyphenyl)propanoic acid
  SITE 1..6: note = Cyclic
KYWXTY

SEQ ID NO: 105 - protein, 6 aa, synthetic construct
  SITE 1: note = methyl-homocysteine
  SITE 3: note = (2R,3S)-2-amino-3-(1H-indol-3-yl)butanoic acid
  SITE 4: note = 2-amino-3-(4-(aminomethyl)phenyl)propanoic acid
  SITE 1..6: note = Cyclic
XYWFTF

SEQ ID NO: 106 - protein, 6 aa, synthetic construct
  SITE 1: note = methyl-homocysteine
  SITE 3: note = (2R,3S)-2-amino-3-(1H-indol-3-yl)butanoic acid
  SITE 4: note = N6-isopropyl-lysine
  SITE 1..6: note = Cyclic
XYWKTF

SEQ ID NO: 107 - protein, 6 aa, synthetic construct
  SITE 1: note = methyl-homocysteine
  SITE 3: note = D-tryptophan
  SITE 4: note = O-(azetidin-3-yl)-serine
  SITE 1..6: note = Cyclic
XYWSTF

SEQ ID NO: 108 - protein, 6 aa, synthetic construct
  SITE 1: note = methyl-homocysteine
  SITE 3: note = D-tryptophan
  SITE 4: note = 2-amino-5-hydrazineylpentanoic acid
  SITE 1..6: note = Cyclic
XYWXTF

SEQ ID NO: 109 - protein, 6 aa, synthetic construct
  SITE 1: note = methyl-homocysteine
  SITE 3: note = (2S,3R)-2-amino-3-(1H-indol-3-yl)butanoic acid
  SITE 1..6: note = Cyclic
XYWKTF

SEQ ID NO: 110 - protein, 6 aa, synthetic construct
  SITE 1: note = methyl-lysine
  SITE 3: note = (2S,3R)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 111 - protein, 6 aa, synthetic construct
  SITE 1: note = methyl-homocysteine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
XYWXTF

SEQ ID NO: 112 - protein, 6 aa, synthetic construct
  SITE 1: note = methyl-homocysteine
  SITE 3: note = (R)-2-amino-3-(6-methyl-1H-indol-3-yl)propanoic acid
  SITE 4: note = 2-amino-3-(piperidin-4-yl)propanoic acid
  SITE 1..6: note = Cyclic
XYWXTF

SEQ ID NO: 113 - protein, 6 aa, synthetic construct
  SITE 1: note = methyl-homocysteine
  SITE 3: note = (R)-2-amino-3-(5-methyl-1H-indol-3-yl)propanoic acid
  SITE 4: note = 2-amino-3-(piperidin-4-yl)propanoic acid
  SITE 1..6: note = Cyclic
XYWXTF

SEQ ID NO: 114 - protein, 6 aa, synthetic construct
  SITE 1: note = methyl-homocysteine
  SITE 3: note = (R)-2-amino-3-(5-chloro-1H-indol-3-yl)propanoic acid
  SITE 4: note = 2-amino-3-(piperidin-4-yl)propanoic acid
  SITE 1..6: note = Cyclic
XYWXTF

SEQ ID NO: 115 - protein, 6 aa, synthetic construct
  SITE 1: note = methyl-homocysteine
  SITE 3: note = (R)-2-amino-3-(5-methoxy-1H-indol-3-yl)propanoic acid
  SITE 4: note = 2-amino-3-(piperidin-4-yl)propanoic acid
  SITE 1..6: note = Cyclic
XYWXTF

SEQ ID NO: 116 - protein, 6 aa, synthetic construct
  SITE 1: note = O-(2-aminoethyl)-N-methyl-serine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 117 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-4-(azetidin-3-yl)butanoic acid
  SITE 1..6: note = Cyclic
KYWATF

SEQ ID NO: 118 - protein, 6 aa, synthetic construct
  SITE 1: note = methyl-lysine
  SITE 2: note = 2-amino-3-(4-methoxyphenyl)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KFWXTF

SEQ ID NO: 119 - protein, 6 aa, synthetic construct
  SITE 1: note = 3-amino-2-(methylamino)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 120 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 6: note = 2-amino-3-cyclohexylpropanoic acid
  SITE 1..6: note = Cyclic
KYWXTX

SEQ ID NO: 121 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTS

SEQ ID NO: 122 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 6: note = 2-amino-3-(pyridin-4-yl)propanoic acid
  SITE 1..6: note = Cyclic
KYWXTF

SEQ ID NO: 123 - protein, 6 aa, synthetic construct
  SITE 1: note = methyl-lysine
  SITE 3: note = (2R,3S)-2-amino-3-(1H-indol-3-yl)butanoic acid
  SITE 4: note = N6-methyl-lysine
  SITE 1..6: note = Cyclic
KYWKTF

SEQ ID NO: 124 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
KYWXTF

SEQ ID NO: 125 - protein, 6 aa, synthetic construct
  SITE 1: note = methyl-homocysteine
  SITE 2: note = 2-amino-3-(3-methoxyphenyl)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XFWXTF

SEQ ID NO: 126 - protein, 6 aa, synthetic construct
  SITE 1: note = methyl-lysine
  SITE 3: note = (R)-2,3,4,9-tetrahydro-1H-pyrido[3,4-b]indole-3-car boxylic acid
  SITE 4: note = 2-amino-3-(piperidin-4-yl)propanoic acid
  SITE 1..6: note = Cyclic
KYXXTF

SEQ ID NO: 127 - protein, 6 aa, synthetic construct
  SITE 1: note = methyl-lysine
  SITE 3: note = (2R,3S)-2-amino-3-(1H-indol-3-yl)butanoic acid
  SITE 4: note = amino(4-aminobutyl)carbamic acid
  SITE 1..6: note = Cyclic
KYWKTF

SEQ ID NO: 128 - protein, 6 aa, synthetic construct
  SITE 1: note = methyl-homocysteine
  SITE 3: note = (R)-2-amino-3-(7-methyl-1H-indol-3-yl)propanoic acid
  SITE 4: note = 2-amino-3-(piperidin-4-yl)propanoic acid
  SITE 1..6: note = Cyclic
XYWXTF

SEQ ID NO: 129 - protein, 6 aa, synthetic construct
  SITE 1: note = methyl-lysine
  SITE 2: note = 2-amino-3-(4-phenoxyphenyl)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KFWXTF

SEQ ID NO: 130 - protein, 6 aa, synthetic construct
  SITE 1: note = methyl-lysine
  SITE 2: note = 2-amino-3,3-diphenylpropanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KFWXTF

SEQ ID NO: 131 - protein, 6 aa, synthetic construct
  SITE 1: note = methyl-homocysteine
  SITE 3: note = D-tryptophan
  SITE 4: note = 2,6-diamino-6-hydroxyhexanoic acid
  SITE 1..6: note = Cyclic
XYWXTF

SEQ ID NO: 132 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXAF

SEQ ID NO: 133 - protein, 6 aa, synthetic construct
  SITE 1: note = methyl-homocysteine
  SITE 4: note = 2,6-diamino-6-hydroxyhexanoic acid
  SITE 1..6: note = Cyclic
XYWXTF

SEQ ID NO: 134 - protein, 6 aa, synthetic construct
  SITE 1: note = methyl-lysine
  SITE 3: note = (2R,3S)-2-amino-3-(1H-indol-3-yl)butanoic acid
  SITE 4: note = 2-amino-3-(4-aminocyclohexyl)propanoic acid
  SITE 1..6: note = Cyclic
KYWXTF

SEQ ID NO: 135 - protein, 6 aa, synthetic construct
  SITE 1: note = (4-aminobutyl)glycine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 136 - protein, 6 aa, synthetic construct
  SITE 1: note = L-hydroxyproline
  SITE 2: note = (R)-2-amino-2-phenylacetic acid
  SITE 3: note = (2R,3S)-2-amino-3-(1H-indol-3-yl)butanoic acid
  SITE 4: note = 2-amino-3-(piperidin-4-yl)propanoic acid
  SITE 5: note = 2-amino-3-(4-(benzyloxy)phenyl)propanoic acid
  SITE 1..6: note = Cyclic
PXWXYF

SEQ ID NO: 137 - protein, 6 aa, synthetic construct
  SITE 1: note = L-hydroxyproline
  SITE 3: note = (2R,3S)-2-amino-3-(1H-indol-3-yl)butanoic acid
  SITE 4: note = 2-amino-3-(piperidin-4-yl)propanoic acid
  SITE 5: note = 2-amino-3-(4-(benzyloxy)phenyl)propanoic acid
  SITE 1..6: note = Cyclic
XYWXYF

SEQ ID NO: 138 - protein, 6 aa, synthetic construct
  SITE 1: note = L-hydroxyproline
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 139 - protein, 6 aa, synthetic construct
  SITE 1: note = 7-amino-2-(methylamino)heptanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 140 - protein, 6 aa, synthetic construct
  SITE 1: note = methyl-lysine
  SITE 3: note = (R)-2-amino-3-(1H-pyrrolo[3,2-b]pyridin-3-yl)propan oic acid
  SITE 4: note = 2-amino-3-(piperidin-4-yl)propanoic acid
  SITE 1..6: note = Cyclic
KYWXTF

SEQ ID NO: 141 - protein, 6 aa, synthetic construct
  SITE 1: note = NMe-N6-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 142 - protein, 6 aa, synthetic construct
  SITE 1: note = Acetylated NMe-Lys
  SITE 3: note = D-tryptophan
  SITE 1..6: note = Cyclic
KYWKVF

SEQ ID NO: 143 - protein, 6 aa, synthetic construct
  SITE 1: note = Acetylated methyl-D-lysine
  SITE 3: note = D-tryptophan
  SITE 1..6: note = Cyclic
KYWKVF

SEQ ID NO: 144 - protein, 6 aa, synthetic construct
  SITE 1: note = Acetylated methyl-lysine
  SITE 3: note = D-tryptophan
  SITE 1..6: note = Cyclic
KYWKTF

SEQ ID NO: 145 - protein, 6 aa, synthetic construct
  SITE 1: note = DOTA-Ahx-methyl-lysine
  SITE 3: note = D-tryptophan
  SITE 1..6: note = Cyclic
KYWKVF

SEQ ID NO: 146 - protein, 6 aa, synthetic construct
  SITE 1: note = DOTA-Ahx-NMe-Orn
  SITE 3: note = D-tryptophan
  SITE 1..6: note = Cyclic
XYWKVF

SEQ ID NO: 147 - protein, 6 aa, synthetic construct
  SITE 1: note = DOTA-Dah-COCH2-NMe-Cys
  SITE 3: note = D-tryptophan
  SITE 1..6: note = Cyclic
CYWKVF

SEQ ID NO: 148 - protein, 6 aa, synthetic construct
  SITE 1: note = DOTA-Dah-COCH2-methyl-homocysteine
  SITE 3: note = D-tryptophan
  SITE 1..6: note = Cyclic
XYWKVF

SEQ ID NO: 149 - protein, 6 aa, synthetic construct
  SITE 1: note = DOTA-Dah-NMe-Glu
  SITE 3: note = D-tryptophan
  SITE 1..6: note = Cyclic
EYWKVF

SEQ ID NO: 150 - protein, 6 aa, synthetic construct
  SITE 1: note = DOTA-Dah-NMe-hGlu
  SITE 3: note = D-tryptophan
  SITE 1..6: note = Cyclic
EYWKVF

SEQ ID NO: 151 - protein, 6 aa, synthetic construct
  SITE 1: note = DOTA-Dah-COCH2-NMe-Cys
  SITE 2: note = 2-amino-3-(pyridin-3-yl)propanoic acid
  SITE 3: note = D-tryptophan
  SITE 1..6: note = Cyclic
CFWKTF

SEQ ID NO: 152 - protein, 6 aa, synthetic construct
  SITE 1: note = DOTA-Dah-COCH2-NMe-Cys
  SITE 2: note = 2-amino-3-(pyridin-4-yl)propanoic acid
  SITE 3: note = D-tryptophan
  SITE 1..6: note = Cyclic
CFWKTF

SEQ ID NO: 153 - protein, 6 aa, synthetic construct
  SITE 1: note = DOTA-Dah-COCH2-methyl-homocysteine
  SITE 2: note = 2-amino-3-(pyridin-3-yl)propanoic acid
  SITE 3: note = D-tryptophan
  SITE 1..6: note = Cyclic
XFWKTF

SEQ ID NO: 154 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = D-tryptophan
  SITE 1..6: note = Cyclic
XFWKTF

SEQ ID NO: 155 - protein, 6 aa, synthetic construct
  SITE 1: note = DOTA-Dah-COCH2-NMe-Amp
  SITE 3: note = D-tryptophan
  SITE 1..6: note = Cyclic
XYWKTF

SEQ ID NO: 156 - protein, 6 aa, synthetic construct
  SITE 1: note = DOTA-Ahx-NMe-hLys
  SITE 3: note = D-tryptophan
  SITE 1..6: note = Cyclic
KYWKTF

SEQ ID NO: 157 - protein, 6 aa, synthetic construct
  SITE 1: note = DOTA-Dah-COCH2-methyl-homocysteine
  SITE 2: note = (2S,3S)-2-amino-3-phenylbutanoic acid
  SITE 3: note = D-tryptophan
  SITE 1..6: note = Cyclic
XFWKTF

SEQ ID NO: 158 - protein, 6 aa, synthetic construct
  SITE 1: note = DOTA-Dah-COCH2-methyl-homocysteine
  SITE 2: note = (2S,3R)-2-amino-3-phenylbutanoic acid
  SITE 3: note = D-tryptophan
  SITE 1..6: note = Cyclic
XFWKTF

SEQ ID NO: 159 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 1..6: note = Cyclic
XYWKTF

SEQ ID NO: 160 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTF

SEQ ID NO: 161 - protein, 6 aa, synthetic construct
  SITE 1: note = DOTA-Dah-COCH2-NMe-Cys
  SITE 3: note = D-tryptophan
  SITE 1..6: note = Cyclic
CYWKTF

SEQ ID NO: 162 - protein, 6 aa, synthetic construct
  SITE 1: note = DOTA-TXA-methyl-lysine
  SITE 3: note = D-tryptophan
  SITE 1..6: note = Cyclic
KYWKTF

SEQ ID NO: 163 - protein, 6 aa, synthetic construct
  SITE 1: note = DOTA-Aeea-methyl-lysine
  SITE 3: note = D-tryptophan
  SITE 1..6: note = Cyclic
KYWKTF

SEQ ID NO: 164 - protein, 6 aa, synthetic construct
  SITE 1: note = DOTA-Dah-COCH2-methyl-homocysteine
  SITE 3: note = D-tryptophan
  SITE 6: note = 2-amino-3-(pyridin-3-yl)propanoic acid
  SITE 1..6: note = Cyclic
XYWKTF

SEQ ID NO: 165 - protein, 6 aa, synthetic construct
  SITE 1: note = DOTA-Dah-COCH2-methyl-homocysteine
  SITE 3: note = D-tryptophan
  SITE 6: note = 2-amino-3-(pyridin-4-yl)propanoic acid
  SITE 1..6: note = Cyclic
XYWKTF

SEQ ID NO: 166 - protein, 6 aa, synthetic construct
  SITE 1: note = DOTA-Dah-COCH2-methyl-homocysteine
  SITE 3: note = D-tryptophan
  SITE 6: note = (2S,3S)-2-amino-3-phenylbutanoic acid
  SITE 1..6: note = Cyclic
XYWKTF

SEQ ID NO: 167 - protein, 6 aa, synthetic construct
  SITE 1: note = DOTA-Dah-COCH2-methyl-homocysteine
  SITE 3: note = D-tryptophan
  SITE 6: note = 3N-Tyr
  SITE 1..6: note = Cyclic
XYWKTY

SEQ ID NO: 168 - protein, 6 aa, synthetic construct
  SITE 1: note = DOTA-Dah-COCH2-methyl-homocysteine
  SITE 2: note = 5FY
  SITE 3: note = D-tryptophan
  SITE 1..6: note = Cyclic
XYWKTF

SEQ ID NO: 169 - protein, 6 aa, synthetic construct
  SITE 1: note = DOTA-Dah-COCH2-methyl-homocysteine
  SITE 3: note = D-tryptophan
  SITE 6: note = (2S,3R)-2-amino-3-phenylbutanoic acid
  SITE 1..6: note = Cyclic
XYWKTF

SEQ ID NO: 170 - protein, 6 aa, synthetic construct
  SITE 1: note = DOTA-methyl-lysine
  SITE 3: note = D-tryptophan
  SITE 1..6: note = Cyclic
KYWKTF

SEQ ID NO: 171 - protein, 6 aa, synthetic construct
  SITE 1: note = DOTA-Aeea-methyl-lysine
  SITE 3: note = D-tryptophan
  SITE 1..6: note = Cyclic
KYWKTF

SEQ ID NO: 172 - protein, 6 aa, synthetic construct
  SITE 1: note = DOTA-Aoc-methyl-lysine
  SITE 3: note = D-tryptophan
  SITE 1..6: note = Cyclic
KYWKTF

SEQ ID NO: 173 - protein, 6 aa, synthetic construct
  SITE 1: note = DOTA-Asu-methyl-lysine
  SITE 3: note = D-tryptophan
  SITE 1..6: note = Cyclic
KYWKTF

SEQ ID NO: 174 - protein, 6 aa, synthetic construct
  SITE 1: note = DOTA-Dah-COCH2-methyl-homocysteine
  SITE 2: note = 2-amino-3-(3-carbamoylphenyl)propanoic acid
  SITE 3: note = D-tryptophan
  SITE 1..6: note = Cyclic
XFWKTF

SEQ ID NO: 175 - protein, 6 aa, synthetic construct
  SITE 1: note = DOTA-Dah-COCH2-methyl-homocysteine
  SITE 2: note = 2-amino-3-(2-carbamoylphenyl)propanoic acid
  SITE 3: note = D-tryptophan
  SITE 1..6: note = Cyclic
XFWKTF

SEQ ID NO: 176 - protein, 6 aa, synthetic construct
  SITE 1: note = DOTA-Dah-COCH2-methyl-homocysteine
  SITE 3: note = D-tryptophan
  SITE 6: note = 2-amino-3-(3-carbamoylphenyl)propanoic acid
  SITE 1..6: note = Cyclic
XYWKTF

SEQ ID NO: 177 - protein, 6 aa, synthetic construct
  SITE 1: note = DOTA-Dah-COCH2-methyl-homocysteine
  SITE 3: note = D-6F-Trp
  SITE 1..6: note = Cyclic
XYWKTF

SEQ ID NO: 178 - protein, 6 aa, synthetic construct
  SITE 1: note = DOTA-Peg3-COCH2-methyl-homocysteine
  SITE 3: note = D-tryptophan
  SITE 5: note = Alt
  SITE 1..6: note = Cyclic
XYWKXF

SEQ ID NO: 179 - protein, 6 aa, synthetic construct
  SITE 1: note = DOTA-Dah-COCH2CH2-methyl-homocysteine
  SITE 3: note = D-tryptophan
  SITE 1..6: note = Cyclic
XYWKVF

SEQ ID NO: 180 - protein, 6 aa, synthetic construct
  SITE 1: note = DOTA-Hly-COCH2CH2-methyl-homocysteine
  SITE 3: note = D-tryptophan
  SITE 1..6: note = Cyclic
XYWKVF

SEQ ID NO: 181 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 1..6: note = Cyclic
XFWKTF

SEQ ID NO: 182 - protein, 6 aa, synthetic construct
  SITE 1: note = DOTA-Dah-COCH2-methyl-homocysteine
  SITE 2: note = 2-amino-3-(pyridin-3-yl)propanoic acid
  SITE 3: note = (2R,3S)-2-amino-3-(1H-indol-3-yl)butanoic acid
  SITE 1..6: note = Cyclic
XFWKTF

SEQ ID NO: 183 - protein, 6 aa, synthetic construct
  SITE 1: note = DOTA-Dah-COCH2-methyl-homocysteine
  SITE 2: note = (2S,3R)-2-amino-3-phenylbutanoic acid
  SITE 3: note = (2R,3S)-2-amino-3-(1H-indol-3-yl)butanoic acid
  SITE 1..6: note = Cyclic
XFWKTF

SEQ ID NO: 184 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 6: note = 2-amino-3-(pyridin-3-yl)propanoic acid
  SITE 1..6: note = Cyclic
XFWKTF

SEQ ID NO: 185 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 6: note = (2S,3R)-2-amino-3-phenylbutanoic acid
  SITE 1..6: note = Cyclic
XFWKTF

SEQ ID NO: 186 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 6: note = (2S,3S)-2-amino-3-phenylbutanoic acid
  SITE 1..6: note = Cyclic
XFWKTF

SEQ ID NO: 187 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = D-tryptophan
  SITE 6: note = 2-amino-3-(pyridin-4-yl)propanoic acid
  SITE 1..6: note = Cyclic
XFWKTF

SEQ ID NO: 188 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = D-tryptophan
  SITE 6: note = 2-amino-3-(pyridin-3-yl)propanoic acid
  SITE 1..6: note = Cyclic
XFWKTF

SEQ ID NO: 189 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = D-tryptophan
  SITE 6: note = (2S,3S)-2-amino-3-phenylbutanoic acid
  SITE 1..6: note = Cyclic
XFWKTF

SEQ ID NO: 190 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = D-tryptophan
  SITE 6: note = (2S,3R)-2-amino-3-phenylbutanoic acid
  SITE 1..6: note = Cyclic
XFWKTF

SEQ ID NO: 191 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = D-tryptophan
  SITE 6: note = 3N-Tyr
  SITE 1..6: note = Cyclic
XFWKTY

SEQ ID NO: 192 - protein, 6 aa, synthetic construct
  SITE 1: note = DOTA-Dah-COCH2-methyl-homocysteine
  SITE 3: note = D-tryptophan
  SITE 4: note = methyl-lysine
  SITE 1..6: note = Cyclic
XYWKTF

SEQ ID NO: 193 - protein, 6 aa, synthetic construct
  SITE 1: note = DOTA-Dah-COCH2-methyl-homocysteine
  SITE 3: note = methyl-D-tryptophan
  SITE 4: note = methyl-lysine
  SITE 1..6: note = Cyclic
XYWKTF

SEQ ID NO: 194 - protein, 6 aa, synthetic construct
  SITE 1: note = DOTA-Dah-COCH2-methyl-homocysteine
  SITE 3: note = D-tryptophan
  SITE 5: note = Alt
  SITE 1..6: note = Cyclic
XYWKXF

SEQ ID NO: 195 - protein, 6 aa, synthetic construct
  SITE 1: note = DOTA-Dap-COCH2-methyl-homocysteine
  SITE 3: note = D-tryptophan
  SITE 1..6: note = Cyclic
XYWKTF

SEQ ID NO: 196 - protein, 6 aa, synthetic construct
  SITE 1: note = DOTA-Dah-COCH2-NMe-hHcy
  SITE 3: note = D-tryptophan
  SITE 1..6: note = Cyclic
XYWKTF

SEQ ID NO: 197 - protein, 6 aa, synthetic construct
  SITE 1: note = DOTA-Daop-COCH2-methyl-homocysteine
  SITE 3: note = D-tryptophan
  SITE 1..6: note = Cyclic
XYWKTF

SEQ ID NO: 198 - protein, 6 aa, synthetic construct
  SITE 1: note = DOTA-Peg3-COCH2-methyl-homocysteine
  SITE 2: note = 2-amino-3-(pyridin-4-yl)propanoic acid
  SITE 3: note = D-tryptophan
  SITE 1..6: note = Cyclic
XFWKTF

SEQ ID NO: 199 - protein, 6 aa, synthetic construct
  SITE 1: note = DOTA-NMe-HLys
  SITE 3: note = D-tryptophan
  SITE 1..6: note = Cyclic
KYWKTF

SEQ ID NO: 200 - protein, 6 aa, synthetic construct
  SITE 1: note = DOTA-Dah-COCH2-methyl-homocysteine
  SITE 3: note = methyl-D-tryptophan
  SITE 4: note = methyl-lysine
  SITE 6: note = methyl-phenylalanine
  SITE 1..6: note = Cyclic
XYWKTF

SEQ ID NO: 201 - protein, 6 aa, synthetic construct
  SITE 1: note = DOTA-Dah-COCH2-methyl-homocysteine
  SITE 3: note = D-tryptophan
  SITE 5: note = 2-amino-2-cyclobutylacetic acid
  SITE 1..6: note = Cyclic
XYWKXF

SEQ ID NO: 202 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 1..6: note = Cyclic
XYWKVF

SEQ ID NO: 203 - protein, 6 aa, synthetic construct
  SITE 1: note = DOTA-Dah-COCH2-methyl-homocysteine
  SITE 3: note = methyl-D-tryptophan
  SITE 4: note = methyl-lysine
  SITE 1..6: note = Cyclic
XYWKVF

SEQ ID NO: 204 - protein, 6 aa, synthetic construct
  SITE 1: note = DOTA-Dah-COCH2-methyl-homocysteine
  SITE 2: note = 2-amino-3,3-diphenylpropanoic acid
  SITE 3: note = D-tryptophan
  SITE 1..6: note = Cyclic
XFWKVF

SEQ ID NO: 205 - protein, 6 aa, synthetic construct
  SITE 1: note = DOTA-Dah-COCH2-methyl-homocysteine
  SITE 3: note = D-tryptophan
  SITE 6: note = 2-amino-3-(methyl(phenyl)amino)propanoic acid
  SITE 1..6: note = Cyclic
XYWKVX

SEQ ID NO: 206 - protein, 6 aa, synthetic construct
  SITE 1: note = DOTA-Dah-COCH2-methyl-homocysteine
  SITE 3: note = D-tryptophan
  SITE 6: note = 2-amino-3-cyclohexylpropanoic acid
  SITE 1..6: note = Cyclic
XYWKVX

SEQ ID NO: 207 - protein, 6 aa, synthetic construct
  SITE 1: note = DOTA-Dah-COCH2-methyl-homocysteine
  SITE 3: note = D-tryptophan
  SITE 5: note = O-methyl-threonine
  SITE 1..6: note = Cyclic
XYWKXF

SEQ ID NO: 208 - protein, 6 aa, synthetic construct
  SITE 1: note = DOTA-Dah-COCH2-methyl-homocysteine
  SITE 3: note = D-tryptophan
  SITE 5: note = 2-amino-3-cyclobutylpropanoic acid
  SITE 1..6: note = Cyclic
XYWKXF

SEQ ID NO: 209 - protein, 6 aa, synthetic construct
  SITE 1: note = DOTA-Dah-COCH2-methyl-homocysteine
  SITE 3: note = D-tryptophan
  SITE 5: note = 2-amino-2-cyclopentylacetic acid
  SITE 1..6: note = Cyclic
XYWKXF

SEQ ID NO: 210 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTG

SEQ ID NO: 211 - protein, 6 aa, synthetic construct
  SITE 1: note = DOTA-Dah-COCH2-methyl-homocysteine
  SITE 3: note = D-tryptophan
  SITE 6: note = 2-amino-2-phenylacetic acid
  SITE 1..6: note = Cyclic
XYWKTX

SEQ ID NO: 212 - protein, 6 aa, synthetic construct
  SITE 1: note = DOTA-Peg3-COCH2-methyl-homocysteine
  SITE 3: note = D-tryptophan
  SITE 1..6: note = Cyclic
XYWKVF

SEQ ID NO: 213 - protein, 6 aa, synthetic construct
  SITE 1: note = DOTA-Dah-COCH2-methyl-homocysteine
  SITE 2: note = 2-amino-3-(pyridin-4-yl)propanoic acid
  SITE 3: note = (2R,3S)-2-amino-3-(1H-indol-3-yl)butanoic acid
  SITE 1..6: note = Cyclic
XFWKVF

SEQ ID NO: 214 - protein, 6 aa, synthetic construct
  SITE 1: note = DOTA-Dah-COCH2-methyl-homocysteine
  SITE 3: note = D-tryptophan
  SITE 4: note = 2-amino-3-(piperidin-4-yl)propanoic acid
  SITE 1..6: note = Cyclic
XYWXTF

SEQ ID NO: 215 - protein, 6 aa, synthetic construct
  SITE 1: note = DOTA-Dah-COCH2-methyl-homocysteine
  SITE 3: note = D-tryptophan
  SITE 4: note = 2-amino-3-(azetidin-3-yl)propanoic acid
  SITE 1..6: note = Cyclic
XYWXTF

SEQ ID NO: 216 - protein, 6 aa, synthetic construct
  SITE 1: note = DOTA-Dah-COCH2-methyl-homocysteine
  SITE 3: note = D-tryptophan
  SITE 4: note = 2-amino-2-(piperidin-4-yl)acetic acid
  SITE 1..6: note = Cyclic
XYWXTF

SEQ ID NO: 217 - protein, 6 aa, synthetic construct
  SITE 1: note = DOTA-Dah-COCH2-methyl-homocysteine
  SITE 3: note = D-tryptophan
  SITE 6: note = 2-amino-2-cyclopropylacetic acid
  SITE 1..6: note = Cyclic
XYWKTG

SEQ ID NO: 218 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTN

SEQ ID NO: 219 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTH

SEQ ID NO: 220 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTA

SEQ ID NO: 221 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTS

SEQ ID NO: 222 - protein, 6 aa, synthetic construct
  SITE 1: note = DOTA-PEG2-COCH2-methyl-homocysteine
  SITE 3: note = D-tryptophan
  SITE 1..6: note = Cyclic
XYWKTF

SEQ ID NO: 223 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTF

SEQ ID NO: 224 - protein, 6 aa, synthetic construct
  SITE 1: note = DOTA-dLys-COCH2-methyl-homocysteine
  SITE 3: note = D-tryptophan
  SITE 1..6: note = Cyclic
XYWKTF

SEQ ID NO: 225 - protein, 6 aa, synthetic construct
  SITE 1: note = DOTA-gamma dLys-COCH2-methyl-homocysteine
  SITE 3: note = D-tryptophan
  SITE 1..6: note = Cyclic
XYWKTF

SEQ ID NO: 226 - protein, 6 aa, synthetic construct
  SITE 1: note = DOTA-PEG3-COCH2-methyl-homocysteine
  SITE 3: note = D-tryptophan
  SITE 1..6: note = Cyclic
XYWKTF

SEQ ID NO: 227 - protein, 6 aa, synthetic construct
  SITE 1: note = DOTA-PEG3-COCH2-methyl-homocysteine
  SITE 3: note = D-tryptophan
  SITE 1..6: note = Cyclic
XYWKTG

SEQ ID NO: 228 - protein, 6 aa, synthetic construct
  SITE 1: note = DOTA-Dah-COCH2-methyl-homocysteine
  SITE 3: note = methyl-D-tryptophan
  SITE 1..6: note = Cyclic
XYWKTF

SEQ ID NO: 229 - protein, 6 aa, synthetic construct
  SITE 1: note = DOTA-Peg3-COCH2-methyl-homocysteine
  SITE 1..6: note = Cyclic
XYWKTF

SEQ ID NO: 230 - protein, 6 aa, synthetic construct
  SITE 1: note = DOTA-Dah-COCH2-methyl-homocysteine
  SITE 3: note = D-tryptophan
  SITE 6: note = methyl-phenylalanine
  SITE 1..6: note = Cyclic
XYWKTF

SEQ ID NO: 231 - protein, 6 aa, synthetic construct
  SITE 1: note = DOTA-Dah-COCH2-methyl-homocysteine
  SITE 3: note = D-tryptophan
  SITE 1..6: note = Cyclic
XGWKTF

SEQ ID NO: 232 - protein, 6 aa, synthetic construct
  SITE 1: note = DOTA-Dah-COCH2-methyl-homocysteine
  SITE 3: note = D-tryptophan
  SITE 1..6: note = Cyclic
XAWKTF

SEQ ID NO: 233 - protein, 6 aa, synthetic construct
  SITE 1: note = DOTA-Ahx-NMe-Dab
  SITE 3: note = D-tryptophan
  SITE 1..6: note = Cyclic
XYWKTF

SEQ ID NO: 234 - protein, 6 aa, synthetic construct
  SITE 1: note = DOTA-Ahx-NMe-Dap
  SITE 3: note = D-tryptophan
  SITE 1..6: note = Cyclic
XYWKTF

SEQ ID NO: 235 - protein, 6 aa, synthetic construct
  SITE 1: note = DOTA-Ahx-Asp-NMe-Dap
  SITE 3: note = D-tryptophan
  SITE 1..6: note = Cyclic
XYWKTF

SEQ ID NO: 236 - protein, 6 aa, synthetic construct
  SITE 1: note = DOTA-Ahx-Beta-Asp-NMe-Dap
  SITE 3: note = D-tryptophan
  SITE 1..6: note = Cyclic
XYWKTF

SEQ ID NO: 237 - protein, 6 aa, synthetic construct
  SITE 1: note = DOTA-Ahx-D-Asp-NMe-Dap
  SITE 3: note = D-tryptophan
  SITE 1..6: note = Cyclic
XYWKTF

SEQ ID NO: 238 - protein, 6 aa, synthetic construct
  SITE 1: note = DOTA-Dah-Beta D-Asp-NMe-Dap
  SITE 3: note = D-tryptophan
  SITE 1..6: note = Cyclic
XYWKTF

SEQ ID NO: 239 - protein, 6 aa, synthetic construct
  SITE 1: note = DOTA-Peg3-COCH2-methyl-homocysteine
  SITE 3: note = D-tryptophan
  SITE 6: note = O-phenyl-serine
  SITE 1..6: note = Cyclic
XYWKTS

SEQ ID NO: 240 - protein, 6 aa, synthetic construct
  SITE 1: note = DOTA-Dah-COCH2-methyl-homocysteine
  SITE 2: note = 3,5-diF Tyr
  SITE 3: note = D-tryptophan
  SITE 1..6: note = Cyclic
XYWKTF

SEQ ID NO: 241 - protein, 6 aa, synthetic construct
  SITE 1: note = DOTA-Dah-COCH2-methyl-homocysteine
  SITE 3: note = D-tryptophan
  SITE 1..6: note = Cyclic
XYWKAF

SEQ ID NO: 242 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 243 - protein, 6 aa, synthetic construct
  SITE 1: note = DOTA-Dah-COCH2-methyl-homocysteine
  SITE 4: note = 2-amino-3-(piperidin-4-yl)propanoic acid
  SITE 1..6: note = Cyclic
XYWXTF

SEQ ID NO: 244 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 245 - protein, 6 aa, synthetic construct
  SITE 1: note = DOTA-Dah-COCH2-methyl-homocysteine
  SITE 3: note = D-tryptophan
  SITE 4: note = N6-methyl-lysine
  SITE 1..6: note = Cyclic
XYWKTF

SEQ ID NO: 246 - protein, 6 aa, synthetic construct
  SITE 1: note = DOTA-Dah-COCH2-methyl-homocysteine
  SITE 3: note = D-tryptophan
  SITE 4: note = N6,N6-dimethyl-lysine
  SITE 1..6: note = Cyclic
XYWKTF

SEQ ID NO: 247 - protein, 6 aa, synthetic construct
  SITE 1: note = DOTA-Dah-COCH2-methyl-homocysteine
  SITE 3: note = D-tryptophan
  SITE 4: note = 2-amino-3-(piperazin-1-yl)propanoic acid
  SITE 1..6: note = Cyclic
XYWXTF

SEQ ID NO: 248 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTA

SEQ ID NO: 249 - protein, 6 aa, synthetic construct
  SITE 1: note = DOTA-Dah-COCH2-methyl-homocysteine
  SITE 2: note = 2-amino-3,3-diphenylpropanoic acid
  SITE 3: note = D-tryptophan
  SITE 4: note = 2-amino-3-(piperidin-4-yl)propanoic acid
  SITE 1..6: note = Cyclic
XFWXTA

SEQ ID NO: 250 - protein, 6 aa, synthetic construct
  SITE 1: note = DOTA-Dah-COCH2-methyl-homocysteine
  SITE 3: note = D-tryptophan
  SITE 4: note = D-Lysine
  SITE 1..6: note = Cyclic
XYWKTF

SEQ ID NO: 251 - protein, 6 aa, synthetic construct
  SITE 1: note = DOTA-Ahx-NMe-Chg4N
  SITE 3: note = D-tryptophan
  SITE 1..6: note = Cyclic
XYWKTF

SEQ ID NO: 252 - protein, 6 aa, synthetic construct
  SITE 1: note = DOTA-Ahx-NMe-Cha4N
  SITE 3: note = D-tryptophan
  SITE 1..6: note = Cyclic
XYWKTF

SEQ ID NO: 253 - protein, 6 aa, synthetic construct
  SITE 1: note = DOTA-Dah-COCH2-methyl-homocysteine
  SITE 3: note = 4-aza-Trp
  SITE 1..6: note = Cyclic
XYWKTF

SEQ ID NO: 254 - protein, 6 aa, synthetic construct
  SITE 1: note = DOTA-Dah-COCH2-methyl-homocysteine
  SITE 3: note = D-tryptophan
  SITE 1..6: note = Cyclic
XYWKTP

SEQ ID NO: 255 - protein, 6 aa, synthetic construct
  SITE 1: note = DOTA-Dah-COCH2-NMe-D-Hcy
  SITE 3: note = D-tryptophan
  SITE 1..6: note = Cyclic
XYWKTF

SEQ ID NO: 256 - protein, 6 aa, synthetic construct
  SITE 1: note = DOTA-Dah-NMe-Asp
  SITE 3: note = D-tryptophan
  SITE 1..6: note = Cyclic
DYWKTF

SEQ ID NO: 257 - protein, 6 aa, synthetic construct
  SITE 1: note = DOTA-Dah-COCH2-methyl-homocysteine
  SITE 3: note = D-tryptophan
  SITE 4: note = 3-Azetidine-hAla
  SITE 1..6: note = Cyclic
XYWATF

SEQ ID NO: 258 - protein, 6 aa, synthetic construct
  SITE 1: note = DOTA-Dah-COCH2-methyl-homocysteine
  SITE 3: note = D-tryptophan
  SITE 4: note = 4-oxa Lys
  SITE 1..6: note = Cyclic
XYWKTF

SEQ ID NO: 259 - protein, 6 aa, synthetic construct
  SITE 1: note = DOTA-Dah-COCH2-methyl-homocysteine
  SITE 3: note = 2-amino-3-(1H-pyrrolo[3,2-c]pyridin-3-yl)propanoic acid
  SITE 1..6: note = Cyclic
XYWKTF

SEQ ID NO: 260 - protein, 6 aa, synthetic construct
  SITE 1: note = DOTA-Dah-COCH2-methyl-homocysteine
  SITE 3: note = 2-amino-3-(1H-pyrrolo[2,3-c]pyridin-3-yl)propanoic acid
  SITE 1..6: note = Cyclic
XYWKTF

SEQ ID NO: 261 - protein, 6 aa, synthetic construct
  SITE 1: note = DOTA-Dah-COCH2-methyl-homocysteine
  SITE 3: note = 2-amino-3-(1H-pyrrolo[2,3-b]pyridin-3-yl)propanoic acid
  SITE 1..6: note = Cyclic
XYWKTF

SEQ ID NO: 262 - protein, 6 aa, synthetic construct
  SITE 1: note = DOTA-methyl-lysine
  SITE 3: note = D-tryptophan
  SITE 4: note = 2-amino-3-(piperidin-4-yl)propanoic acid
  SITE 1..6: note = Cyclic
KYWXTY

SEQ ID NO: 263 - protein, 6 aa, synthetic construct
  SITE 1: note = DOTA-methyl-lysine
  SITE 3: note = D-tryptophan
  SITE 4: note = 2-amino-3-(piperidin-4-yl)propanoic acid
  SITE 6: note = 2-amino-3-(3-hydroxyphenyl)propanoic acid
  SITE 1..6: note = Cyclic
KYWXTY

SEQ ID NO: 264 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 6: note = 2-amino-3-(3-hydroxyphenyl)propanoic acid
  SITE 1..6: note = Cyclic
KYWXTY

SEQ ID NO: 265 - protein, 6 aa, synthetic construct
  SITE 1: note = DOTA-en-COCH2-methyl-homocysteine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 266 - protein, 6 aa, synthetic construct
  SITE 1: note = DOTA-PEG3-COCH2-methyl-homocysteine
  SITE 4: note = 2-amino-3-(piperidin-4-yl)propanoic acid
  SITE 1..6: note = Cyclic
XYWXTF

SEQ ID NO: 267 - protein, 6 aa, synthetic construct
  SITE 1: note = DOTA-PEG3-COCH2-methyl-homocysteine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 268 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 4: note = 2-amino-3-(4-(aminomethyl)phenyl)propanoic acid
  SITE 1..6: note = Cyclic
XYWFTF

SEQ ID NO: 269 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 4: note = N6-isopropyl-lysine
  SITE 1..6: note = Cyclic
XYWKTF

SEQ ID NO: 270 - protein, 6 aa, synthetic construct
  SITE 1: note = DOTA-Dah-COCH2-methyl-homocysteine
  SITE 3: note = D-tryptophan
  SITE 4: note = O-(azetidin-3-yl)-serine
  SITE 1..6: note = Cyclic
XYWSTF

SEQ ID NO: 271 - protein, 6 aa, synthetic construct
  SITE 1: note = DOTA-Dah-COCH2-methyl-homocysteine
  SITE 3: note = D-tryptophan
  SITE 4: note = 2-amino-5-hydrazineylpentanoic acid
  SITE 1..6: note = Cyclic
XYWXTF

SEQ ID NO: 272 - protein, 6 aa, synthetic construct
  SITE 1: note = DOTA-Dah-COCH2-methyl-homocysteine
  SITE 3: note = (2S,3R)-2-amino-3-(1H-indol-3-yl)butanoic acid
  SITE 1..6: note = Cyclic
XYWKTF

SEQ ID NO: 273 - protein, 6 aa, synthetic construct
  SITE 1: note = DOTA-methyl-lysine
  SITE 3: note = (2S,3R)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 274 - protein, 6 aa, synthetic construct
  SITE 1: note = DOTA-Dah-COCH2-methyl-homocysteine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
XYWXTF

SEQ ID NO: 275 - protein, 6 aa, synthetic construct
  SITE 1: note = DOTA-Dah-COCH2-methyl-homocysteine
  SITE 3: note = (6-Me) D-Trp
  SITE 4: note = 2-amino-3-(piperidin-4-yl)propanoic acid
  SITE 1..6: note = Cyclic
XYWXTF

SEQ ID NO: 276 - protein, 6 aa, synthetic construct
  SITE 1: note = DOTA-Dah-COCH2-methyl-homocysteine
  SITE 3: note = (5-Me) D-Trp
  SITE 4: note = 2-amino-3-(piperidin-4-yl)propanoic acid
  SITE 1..6: note = Cyclic
XYWXTF

SEQ ID NO: 277 - protein, 6 aa, synthetic construct
  SITE 1: note = DOTA-Dah-COCH2-methyl-homocysteine
  SITE 3: note = (5-Cl) D-Trp
  SITE 4: note = 2-amino-3-(piperidin-4-yl)propanoic acid
  SITE 1..6: note = Cyclic
XYWXTF

SEQ ID NO: 278 - protein, 6 aa, synthetic construct
  SITE 1: note = DOTA-Dah-COCH2-methyl-homocysteine
  SITE 3: note = (5-MeO) D-Trp
  SITE 4: note = 2-amino-3-(piperidin-4-yl)propanoic acid
  SITE 1..6: note = Cyclic
XYWXTF

SEQ ID NO: 279 - protein, 6 aa, synthetic construct
  SITE 1: note = DOTA-O-(2-aminoethyl)-N-methyl-serine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 280 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 3-Azetidine-hAla
  SITE 1..6: note = Cyclic
KYWATF

SEQ ID NO: 281 - protein, 6 aa, synthetic construct
  SITE 1: note = DOTA-methyl-lysine
  SITE 2: note = 2-amino-3-(4-methoxyphenyl)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KFWXTF

SEQ ID NO: 282 - protein, 6 aa, synthetic construct
  SITE 1: note = DOTA-Ahx-Asp-NMe-Dap
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 283 - protein, 6 aa, synthetic construct
  SITE 1: note = DOTA-Asp-NMe-Dap
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 284 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 6: note = 2-amino-3-cyclohexylpropanoic acid
  SITE 1..6: note = Cyclic
KYWXTX

SEQ ID NO: 285 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTS

SEQ ID NO: 286 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 6: note = 2-amino-3-(pyridin-4-yl)propanoic acid
  SITE 1..6: note = Cyclic
KYWXTF

SEQ ID NO: 287 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = N6-methyl-lysine
  SITE 1..6: note = Cyclic
KYWKTF

SEQ ID NO: 288 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
KYWXTF

SEQ ID NO: 289 - protein, 6 aa, synthetic construct
  SITE 1: note = DOTA-Dah-COCH2-methyl-homocysteine
  SITE 3: note = (5-Cl) D-Trp
  SITE 4: note = 2-amino-3-(piperidin-4-yl)propanoic acid
  SITE 1..6: note = Cyclic
XYWXTF

SEQ ID NO: 290 - protein, 6 aa, synthetic construct
  SITE 1: note = DOTA-Dah-COCH2-methyl-homocysteine
  SITE 2: note = 2-amino-3-(3-methoxyphenyl)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XFWXTF

SEQ ID NO: 291 - protein, 6 aa, synthetic construct
  SITE 1: note = DOTA-methyl-lysine
  SITE 3: note = (R)-2,3,4,9-tetrahydro-1H-pyrido[3,4-b]indole-3-car boxylic acid
  SITE 4: note = 2-amino-3-(piperidin-4-yl)propanoic acid
  SITE 1..6: note = Cyclic
KYXXTF

SEQ ID NO: 292 - protein, 6 aa, synthetic construct
  SITE 1: note = DOTA-Glu-3-amino-2-(methylamino)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 293 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = amino(4-aminobutyl)carbamic acid
  SITE 1..6: note = Cyclic
KYWKTF

SEQ ID NO: 294 - protein, 6 aa, synthetic construct
  SITE 1: note = DOTA-Dah-COCH2-methyl-homocysteine
  SITE 3: note = (7-Me) D-Trp
  SITE 4: note = 2-amino-3-(piperidin-4-yl)propanoic acid
  SITE 1..6: note = Cyclic
XYWXTF

SEQ ID NO: 295 - protein, 6 aa, synthetic construct
  SITE 1: note = DOTA-methyl-lysine
  SITE 2: note = 2-amino-3-(4-phenoxyphenyl)propanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 296 - protein, 6 aa, synthetic construct
  SITE 1: note = DOTA-methyl-lysine
  SITE 2: note = 2-amino-3,3-diphenylpropanoic acid
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KFWXTF

SEQ ID NO: 297 - protein, 6 aa, synthetic construct
  SITE 1: note = (R/S)-NODAGA-Ga-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 298 - protein, 6 aa, synthetic construct
  SITE 1: note = NOTA-Ga-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 299 - protein, 6 aa, synthetic construct
  SITE 1: note = DOTA-Dah-COCH2-methyl-homocysteine
  SITE 3: note = D-tryptophan
  SITE 4: note = (D/L)Hly
  SITE 1..6: note = Cyclic
XYWXTF

SEQ ID NO: 300 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXAF

SEQ ID NO: 301 - protein, 6 aa, synthetic construct
  SITE 1: note = DOTA-Dah-COCH2-methyl-homocysteine
  SITE 4: note = 2,6-diamino-6-hydroxyhexanoic acid
  SITE 1..6: note = Cyclic
XYWXTF

SEQ ID NO: 302 - protein, 6 aa, synthetic construct
  SITE 1: note = DOTA-methyl-lysine
  SITE 3: note = (2R,3S)-2-amino-3-(1H-indol-3-yl)butanoic acid
  SITE 4: note = 2-amino-3-(4-aminocyclohexyl)propanoic acid
  SITE 1..6: note = Cyclic
KYWXTF

SEQ ID NO: 303 - protein, 6 aa, synthetic construct
  SITE 1: note = DOTA-(4-aminobutyl)glycine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 304 - protein, 6 aa, synthetic construct
  SITE 1: note = DOTA-CDAE-L-hydroxyproline
  SITE 2: note = (R)-2-amino-2-phenylacetic acid
  SITE 3: note = (2R,3S)-2-amino-3-(1H-indol-3-yl)butanoic acid
  SITE 4: note = 2-amino-3-(piperidin-4-yl)propanoic acid
  SITE 5: note = 2-amino-3-(4-(benzyloxy)phenyl)propanoic acid
  SITE 1..6: note = Cyclic
PXWXYF

SEQ ID NO: 305 - protein, 6 aa, synthetic construct
  SITE 1: note = DOTA-CDAE-L-hydroxyproline
  SITE 3: note = (2R,3S)-2-amino-3-(1H-indol-3-yl)butanoic acid
  SITE 4: note = 2-amino-3-(piperidin-4-yl)propanoic acid
  SITE 5: note = 2-amino-3-(4-(benzyloxy)phenyl)propanoic acid
  SITE 1..6: note = Cyclic
XYWXYF

SEQ ID NO: 306 - protein, 6 aa, synthetic construct
  SITE 1: note = DOTA-CDAE-L-hydroxyproline
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
XYWXTF

SEQ ID NO: 307 - protein, 6 aa, synthetic construct
  SITE 1: note = DOTA-Gly-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 308 - protein, 6 aa, synthetic construct
  SITE 1: note = DOTA-Beta-Ala-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 309 - protein, 6 aa, synthetic construct
  SITE 1: note = DOTA-GABA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 310 - protein, 6 aa, synthetic construct
  SITE 1: note = DOTA-Apa-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 311 - protein, 6 aa, synthetic construct
  SITE 1: note = ((R)-NODAGA-Ga)-methyl-lysine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
KYWXTF

SEQ ID NO: 312 - protein, 6 aa, synthetic construct
  SITE 1: note = ((R)-NODAGA-Ga)-methyl-lysine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
KYWXTF

SEQ ID NO: 313 - protein, 6 aa, synthetic construct
  SITE 1: note = DOTA-TXA-methyl-lysine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
KYWXTF

SEQ ID NO: 314 - protein, 6 aa, synthetic construct
  SITE 1: note = DOTA-NMe-hLys
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 315 - protein, 6 aa, synthetic construct
  SITE 1: note = DOTA-4PipCA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 316 - protein, 6 aa, synthetic construct
  SITE 1: note = DOTA-3AzeCA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 317 - protein, 6 aa, synthetic construct
  SITE 1: note = DOTA-4Pipaa-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 318 - protein, 6 aa, synthetic construct
  SITE 1: note = DOTA-3Azeaa-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 319 - protein, 6 aa, synthetic construct
  SITE 1: note = DOTA-TXA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 320 - protein, 6 aa, synthetic construct
  SITE 1: note = DOTA-TXA-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 321 - protein, 6 aa, synthetic construct
  SITE 1: note = ((R)-NODAGA-Ga)-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 322 - protein, 6 aa, synthetic construct
  SITE 1: note = NOTA-Ga-methyl-lysine
  SITE 3: note = (2R,3S)-2-amino-3-(1H-indol-3-yl)butanoic acid
  SITE 4: note = 2-amino-3-(piperazin-1-yl)propanoic acid
  SITE 1..6: note = Cyclic
KYWXTF

SEQ ID NO: 323 - protein, 6 aa, synthetic construct
  SITE 1: note = DOTA-methyl-lysine
  SITE 3: note = (R)-2-amino-3-(1H-indazol-3-yl)propanoic acid
  SITE 4: note = 2-amino-3-(piperidin-4-yl)propanoic acid
  SITE 1..6: note = Cyclic
KYWXTF

SEQ ID NO: 324 - protein, 6 aa, synthetic construct
  SITE 1: note = DOTA-2.2.2.Octadma-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 325 - protein, 6 aa, synthetic construct
  SITE 1: note = DOTA-NMe-N6-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 326 - protein, 6 aa, synthetic construct
  SITE 1: note = DOTA-triazole-methyl-lysine
  SITE 3: note = (2R,3S)-2-amino-3-(1H-indol-3-yl)butanoic acid
  SITE 4: note = 2-amino-3-(piperidin-4-yl)propanoic acid
  SITE 1..6: note = Cyclic
KYWXTF

SEQ ID NO: 327 - protein, 5 aa, synthetic construct
EAAAK

SEQ ID NO: 328 - protein, 30 aa, synthetic construct
  VARIANT 6..30: note = GGGGS repeats may be deleted
GGGGSGGGGSGGGGSGGGGSGGGGSGGGGS

SEQ ID NO: 329 - protein, 6 aa, synthetic construct
  SITE 1: note = threonine(tertiary butyl)
  SITE 3: note = methyl-homocysteine(trityl)
  SITE 4: note = Tyrosine(tertiary butyl)
  SITE 5: note = D-tryptophan(tert-butyloxycarbonyl)
  SITE 6: note = Lysine(tert-butyloxycarbonyl)
TFCYWK

SEQ ID NO: 330 - protein, 6 aa, synthetic construct
  SITE 1: note = methyl-homocysteine(trityl)
  SITE 2: note = Tyrosine(tertiary butyl)
  SITE 3: note = D-tryptophan(tert-butyloxycarbonyl)
  SITE 4: note = Lysine(tert-butyloxycarbonyl)
  SITE 5: note = threonine(tertiary butyl)
  SITE 1..6: note = Cyclic
XYWKTF

SEQ ID NO: 331 - protein, 6 aa, synthetic construct
  SITE 1: note = Threonine(tertiary butyl)
  SITE 3: note = 4-amino-2-(methylamino)butanoic acid(tert-butyloxycarbonyl)
  SITE 4: note = Tyrosine(tertiary butyl)
  SITE 5: note = D-tryptophan(tert-butyloxycarbonyl)
  SITE 6: note = N-epsilon-1-(4,4-dimethyl-2,6-dioxocyclohex-1-ylide ne)ethyl-L-lysine
XYWKTF

SEQ ID NO: 332 - protein, 6 aa, synthetic construct
  SITE 1: note = 4-amino-2-(methylamino)butanoic acid
  SITE 3: note = D-tryptophan
  SITE 4: note = N-epsilon-1-(4,4-dimethyl-2,6-dioxocyclohex-1-ylide ne)ethyl-L-lysine
  SITE 1..6: note = Cyclic
XYWKTF

SEQ ID NO: 333 - protein, 6 aa, synthetic construct
  SITE 1: note = methyl-L-aspartate
  SITE 3: note = D-tryptophan
  SITE 4: note = N-epsilon-1-(4,4-dimethyl-2,6-dioxocyclohex-1-ylide ne)ethyl-L-lysine
  SITE 1..6: note = Cyclic
DYWKTF

SEQ ID NO: 334 - protein, 6 aa, synthetic construct
  SITE 1: note = S-beta-Me D-Trp
  SITE 2: note = 2-amino-3-(piperidin-4-yl)propanoic acid(tert-butyloxycarbonyl)
  SITE 3: note = threonine(tertiary butyl)
  SITE 5: note = protected proline
  SITE 6: note = Tyrosine(tertiary butyl)
WXTFPY

SEQ ID NO: 335 - protein, 6 aa, synthetic construct
  SITE 1: note = (2R,3S)-2-amino-3-(1H-indol-3-yl)butanoic acid
  SITE 2: note = Cha4N(tert-butyloxycarbonyl)
  SITE 3: note = threonine(tertiary butyl)
  SITE 5: note = protected proline
  SITE 6: note = Tyrosine(tertiary butyl)
  SITE 1..6: note = Cyclic
WXTFPY